(19) United States
(12) Patent Application Publication
(10) Pub. No.: US 2025/0034200 A1
(43) Pub. Date: Jan. 30, 2025

(54) Prodrugs of Neurosteroid Analogs and Uses Thereof

(71) Applicant: Emory University, Atlanta, GA (US)

(72) Inventors: Dennis Liotta, Atlanta, GA (US); Russell Fritzemeier, Atlanta, GA (US); Alet van der Westhuyzen, Atlanta, GA (US); Luke E. Hodson, Atlanta, GA (US)

(21) Appl. No.: 18/709,395

(22) PCT Filed: Nov. 10, 2022
(86) PCT No.: PCT/US2022/049489

§ 371 Date: May 10, 2024

Related U.S. Application Data
(60) Provisional application No. 63/277,902, filed on Nov. 10, 2021.
(60) Provisional application No. 63/322,985, filed on Mar. 23, 2022.

(51) Int. Cl. C07J 41/00; A61K 31/57; A61K 31/58; A61P 25/00; C07J 43/00

(52) U.S. Cl. CPC C07J 41/0066; A61K 31/57; A61K 31/58; A61P 25/00; C07J 41/0033; C07J 43/003

(57) ABSTRACT

Prodrugs of neurosteroid analogs are disclosed. Pharmaceutical formulations containing the prodrugs are also disclosed, Additionally, methods of treating a condition, disorder, or disease using the prodrugs or their pharmaceutical formulations are disclosed. Exemplary conditions, disorders, and diseases relevant to this disclosure include stroke, subarachnoid hemorrhage, cerebral ischemia, cerebral vasospasm, hypoxia, CNS injury, concussion, traumatic brain injury, depression, postpartum depression, epilepsy, seizure disorder, and neurodegenerative disease.

### CROSS-REFERENCE TO RELATED APPLICATIONS

This application claims the benefit of U.S. Provisional Application No. 63/322,985 filed Mar. 23, 2022 and U.S. Provisional Application No. 63/277,902 filed Nov. 10, 2021. The entirety of each of these applications is hereby incorporated by reference for all purposes.

### TECHNICAL FIELD

The present disclosure relates to prodrugs of neurosteroid analogs. It also relates to pharmaceutical formulations of the prodrugs and methods for treating conditions, disorders, or diseases using the prodrugs.

### BACKGROUND

Brain injury, such as traumatic brain injury (TBI) and stroke, affects over two million Americans each year and is a significant health concern worldwide. Following brain injury, a cascade of physiological events leads to neuronal loss. Moreover, brain injury can cause inflammation and edema that further propagate neurological damage, leading to secondary injury that may cause long-term neurological deficits.

TBIs typically result from blunt force head trauma or a penetrating head injury that disrupts the brain's regular functions. TBIs also promote a complex succession of molecular events, resulting in varying degrees of secondary injury in proportion to the severity and frequency of the primary injury. Such secondary injury occurs rapidly after the initial trauma and unfolds over days, weeks, or even months. Stokes typically result from diseases that affect the blood vessels that supply blood to the brain. A stroke occurs when a blood vessel that brings oxygen and nutrients to the brain either ruptures (hemorrhagic stroke) or is occluded by a blood clot or some other mass (ischemic stroke). Ischemic strokes are more prevalent; however, hemorrhagic strokes typically result in more severe injuries and have no approved treatments.

After TBI or stroke, inflammation is a principal cause of secondary injury and long-term neurological deficits. Insults to the brain can trigger an inflammatory immune response and excitotoxicity resulting from disruption of the glutamate, acetylcholine, cholinergic, GABAA, and/or NMDA receptor systems. As a result, cytokines are released and signal the delivery of bloodborne leukocytes to the corresponding injury sites to neutralize potential pathogens and promote tissue repair. However, the powerful inflammatory response has the capacity to cause damages to normal tissue, leading to neuronal loss. In addition to TBI and stroke, inflammation is recognized as a key component of various central nervous system (CNS) disorders and diseases, such as neurodegenerative diseases, including dementia and Alzheimer's disease.

Despite several decades of efforts, few pharmacological agents that significantly improve outcomes after TBI or stroke have been developed (Sauerland, et al., Lancet, 2004, 364, 1291-1292). In recent years, there has been a growing body of experimental evidence demonstrating that neurosteroids, such as progesterone and its metabolites/analogs, are effective neuroprotective agents. Neurosteroids are steroids that can be synthesized in the CNS independent of endocrine sources and display neuroactive effects. Neurosteroids have anti-inflammatory, antioxidant, and neuroprotective roles and engage various neurological targets such as GABA and glutamate receptors, among others.

For example, preclinical and clinical research has shown that progesterone and its metabolites/analogs, such as allopregnanolone, epipregnanolone, pregnenolone, dehydroepiandrosterone, can dramatically reduce cerebral edema, inflammation, tissue necrosis, and programmed cell death (Garcia-Estrada, et al., International Journal of Developmental Neuroscience, 1999, 17, 145-161; Djebaili, et al., J Neurotrauma, 2005, 22, 106-118; Pettus, et al., Brain Res, 2005, 1049, 112-119; Grossman, et al., Brain Res, 2004, 1008, 29-39; He, et al., Exp Neurol, 2004, 189, 404-412; Balan, et al., Scientific Reports, 2019, 9, 1220). In vivo data has demonstrated progesterone's neuroprotective effects in animal models of TBI and stroke (Stein, Annals of the New York Academy of Sciences, 1052:152-169; Wright, et al., Ann. Emerg. Med., 2007, 49, 391; Frechou, et al., Neuropharmacology, 2015, 97, 394-403; Lammerding, et al., Neuroendocrinology, 2016, 103, 460-475; Espinosa-Garcia, et al., International Journal of Molecular Sciences, 2020, 21, 3740).

Brain injury treatments rely on symptom management with the goal of mitigating secondary injury due to inflammation and edema. Not surprisingly, minimizing the time from symptom onset to treatment is considered paramount in reducing the likelihood of long-term damage. Unfortunately, previous investigations into the use of neurosteroids for brain injury treatment typically required administration in a hospital setting, thus losing valuable time before the treatment could be administered. Progesterone, as well as most pregnane and androstane neurosteroids, are insoluble in aqueous formulations and require complicated and time-consuming lipid formulations that preclude use in a prehospital setting. Furthermore, the plasma half-life of neurosteroids is limited, and treatment requires prolonged intravenous infusion or multiple injections, further delaying treatment. These flaws of neurosteroids are believed to contribute to the recent failure of two Phase III clinical trials on the use of progesterone for TBI treatment (Stein, Brain Inj, 2015, 19, 29, 1259-1272).

Therefore, there is an urgent need for pharmacological treatments for brain injury that improve short and long-term neurological outcomes through the provision of trophic support to the surviving brain tissue and/or by enhancement of functional repair and recovery following the initial insult. While neurosteroids are natural metabolites with promising neuroprotective potential, there is an urgent need for improved neurosteroid derivatives that can be administered easily and rapidly for treating brain injury, especially acute brain injury.

### SUMMARY

The present disclosure describes prodrugs of neurosteroid analogs. Generally, the prodrugs have a higher solubility in an aqueous medium than their corresponding neurosteroid analogs. In some embodiments, the prodrugs are capable of self-immolative cleavage in response to environmental pH changes, releasing the corresponding neurosteroid analogs. In some embodiments, the prodrugs are stable in an acidic aqueous medium but exhibit a wide range of release kinetics in human plasma.

In some embodiments, the compounds have a structure of Formula II or a pharmaceutically acceptable salt, hydrate, or hydrated salt of Formula II,

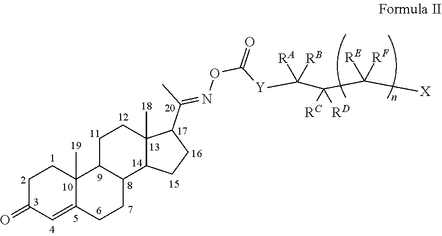

wherein n is 0 or 1; and
wherein:
(1) X is OH or NR¹R²,
Y is O or NR³,
R^{A}, R^{B}, R^{C}, R^{D}, R^{E}, and R^{F} are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R¹, R², and R³ are independently selected from hydrogen, optionally substituted alkyl, optionally substituted heteroalkyl, optionally substituted carbocyclyl, optionally substituted heterocyclyl, optionally substituted aryl, and optionally substituted heteroaryl, with the proviso that at least one of R¹ and R² is hydrogen;
(2) X is NR¹R²,
Y is O or NR³,
R¹ joins R^{C} or R^{E} to form a 4-7 membered, optionally substituted heterocycle,
R² is hydrogen,
R^{A}, R^{B}, R^{C}, R^{D}, R^{E}, and R^{F}, on each occurrence when not joined by R¹ to form the heterocycle, are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R³ is independently selected from hydrogen, optionally substituted alkyl, optionally substituted heteroalkyl, optionally substituted carbocyclyl, optionally substituted heterocyclyl, optionally substituted aryl, and optionally substituted heteroaryl; or
(3) X is OH or NR¹R²,
Y is NR³,
R³ joins R^{A} to form a 4-7 membered, optionally substituted heterocycle,
R^{B}, R^{C}, R^{D}, R^{E}, and R^{F} are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R¹ and R² are independently selected from hydrogen, optionally substituted alkyl, optionally substituted heteroalkyl, optionally substituted carbocyclyl, optionally substituted heterocyclyl, optionally substituted aryl, and optionally substituted heteroaryl, with the proviso that at least one of R¹ and R² is hydrogen.

In some embodiments, n is 0. In some embodiments, n is 1.

In some embodiments, Formula II is in the form of Formula II-1:

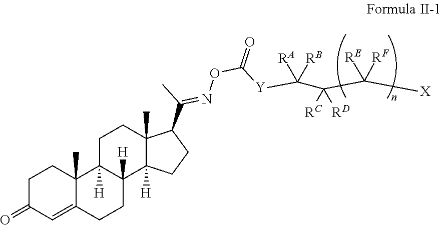

wherein X, Y, R^{A}, R^{B}, R^{C}, R^{D}, R^{E}, R^{E}, and n are the same as those described in Formula II.

Optionally, Formula II and Formula II-1 may have the following features:
X is OH or NR¹R²,
Y is O or NR³,
R^{A}, R^{B}, R^{C}, R^{D}, R^{E}, and R^{F} are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R¹, R², and R³ are independently selected from hydrogen, optionally substituted alkyl, optionally substituted heteroalkyl, optionally substituted carbocyclyl, optionally substituted heterocyclyl, optionally substituted aryl, and optionally substituted heteroaryl, with the proviso that at least one of R¹ and R² is hydrogen.

In some embodiments, X is NR¹R². In some embodiments, R¹ is an optionally substituted C₁-C₄ alkyl, such as —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, or —CH₂CH₂N(CH₃)₃⁺.

In some embodiments, Y is NR³. In some embodiments, R³ is an optionally substituted C₁-C₄ alkyl, such as —CH₃, CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, CH₂CH₂N(CH₃)₂, or —CH₂CH₂N(CH₃)₃⁺. In some embodiments, R³ is optionally substituted carbocyclyl or optionally substituted heterocyclyl, such as

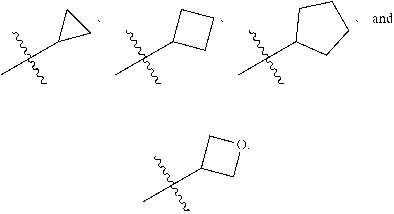

In some embodiments, both R^{A} and R^{B} are hydrogen. In some embodiments, both R^{C} and R^{D} are hydrogen. In some embodiments, when present, both R^{E} and R^{F} are hydrogen.

In some embodiments, n is 0, X is NR¹R², and Y is NR³.

Optionally, Formula II and Formula II-1 may have the following features:
X is NR¹R²,
Y is O or NR³,
R¹ joins R^{C} or R^{E} to form a 4-7 membered, optionally substituted heterocycle,
R² is hydrogen,
R^{A}, R^{B}, R^{C}, R^{D}, R^{E}, and R^{F}, on each occurrence when not joined by R¹ to form the heterocycle, are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R³ is independently selected from hydrogen, optionally substituted alkyl, optionally substituted heteroalkyl, optionally substituted carbocyclyl, optionally substituted heterocyclyl, optionally substituted aryl, and optionally substituted heteroaryl.

In some embodiments, the 4-7 membered, optionally substituted heterocycle is a 5 or 6 membered, optionally substituted heterocycle, such as an optionally substituted pyrrolidine or optionally substituted piperidine.

In some embodiments, Y is NR³. In some embodiments, R³ is an optionally substituted C₁-C₄ alkyl, such as CH₃, —CF₃, —CH₂CH₃, —CH₂CH₂OH, —CH₂CH₂F, —CH₂CF₃, —CH₂OCF₃, CH₂CH₂OCF₃, —CH(CH₃)₂, —CH₂COOH, —CH₂CH₂COOH, CH₂CH(CH₃)₂, —C(CH₃)₃, —CH₂CH₂NH₂, CH₂CH₂NHCH₃, —CH₂CH₂N(CH₃)₂, or —CH₂CH₂N(CH₃)₃⁺. In some embodiments, R³ is optionally substituted carbocyclyl or optionally substituted heterocyclyl, such as

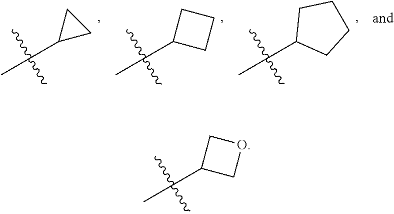

In some embodiments, both R^{A} and R^{B} are hydrogen. In some embodiments, R^{D} is hydrogen. In some embodiments, when present, R^{F} is hydrogen.

In some embodiments, n is 0, and Y is NR³.

In some embodiments, n is 1, and Y is NR³. In some embodiments, n is 1, Y is NR³, and R¹ joins R^{E} to form the 4-7 membered, optionally substituted heterocycle.

Optionally, Formula II and Formula II-1 may have the following features:
X is OH or NR¹R²,
Y is NR³,
R³ joins R^{A} to form a 4-7 membered, optionally substituted heterocycle,
R^{B}, R^{C}, R^{D}, R^{E}, and R^{F} are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R¹ and R² are independently selected from hydrogen, optionally substituted alkyl, optionally substituted heteroalkyl, optionally substituted carbocyclyl, optionally substituted heterocyclyl, optionally substituted aryl, and optionally substituted heteroaryl, with the proviso that at least one of R¹ and R² is hydrogen.

In some embodiments, X is NR 1R². In some embodiments, R¹ is optionally substituted C₁-C₄ alkyl, such as —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, or —CH₂CH₂N(CH₃)₃⁺.

In some embodiments, the 4-7 membered, optionally substituted heterocycle is a 5 or 6 membered, optionally substituted heterocycle, such as an optionally substituted pyrrolidine or optionally substituted piperidine.

In some embodiments, R^{B} is hydrogen. In some embodiments, both R^{C} and R^{D} are hydrogen. In some embodiments, when present, both R^{E} and R^{F} are hydrogen.

In some embodiments, n is 0, and X is NR¹R².

Also disclosed are compositions containing a compound described herein, wherein the compound is in greater than 80%, 85%, 90%, or 95% enantiomeric or diastereomeric excess. In some embodiments, the compound in the compositions is in greater than 95% enantiomeric or diastereomeric excess.

Also disclosed are pharmaceutical formulations of the disclosed compounds or compositions. In general, the pharmaceutical formulations contain a pharmaceutically acceptable excipient. In some embodiments, the pharmaceutical formulations are in a form chosen from tablets, capsules, caplets, pills, beads, granules, particles, powders, gels, creams, solutions, suspensions, emulsions, and nanoparticulate formulations. In some embodiments, the pharmaceutical formulations are oral formulations. In some embodiments, the pharmaceutical formulations are intravenous formulations. In some embodiments, the pharmaceutical formulations are intramuscular formulations. In some embodiments, the pharmaceutical formulations are in the form of a solution, such as an aqueous solution. In some embodiments, the pharmaceutical formulations are in the form of a powder, such as a lyophilized powder.

This disclosure also relates to (1) the compounds, compositions, and pharmaceutical formulations disclosed herein for treatment of a condition, disorder, or disease disclosed herein or use as a medicament, (2) the compounds, compositions, and pharmaceutical formulations disclosed herein for use in the treatment of a condition, disorder, or disease disclosed herein, or (3) the compounds, compositions, and pharmaceutical formulations disclosed herein for the manufacture of a medicament for treatment of a condition, disorder, or disease disclosed herein.

This disclosure also provides methods of treating a condition, disorder, or disease in a subject in need thereof. The method includes administering an effective amount of a compound, composition, or pharmaceutical formulation disclosed herein to the subject. In some embodiments, the compound, composition, or pharmaceutical formulation is administered orally, intravenously, or intramuscularly.

Exemplary conditions, disorders, and diseases relevant to this disclosure include, but are not limited to, stroke, subarachnoid hemorrhage, cerebral ischemia, cerebral vasospasm, hypoxia, CNS injury, concussion, traumatic brain injury, depression, postpartum depression, epilepsy, seizure disorder, and neurodegenerative disease.

### BRIEF DESCRIPTION OF THE SEVERAL VIEWS OF THE DRAWINGS

FIG. 1 illustrates self-immolative of the compounds in response to environmental pH changes.

FIG. 2A illustrates the stability of compound 32b (RGF-PROG-44) in three aqueous mediums-PBS (pH 7.1-7.3), pH 5.5 acetate buffer, and pH 4.0 acetate buffer. The stability is presented by plotting the percentage of remaining compound against time (h).

FIG. 2B illustrates the stability of compound 32j (RGF-PROG-104) in three aqueous mediums-PBS (pH 7.1-7.3), pH 5.5 acetate buffer, and pH 4.0 acetate buffer. The stability is presented by plotting the percentage of remaining compound against time (h).

FIG. 3A illustrates the degradation of compounds 32b (RGF-PROG-44) in human plasma as well as the simultaneous formation of progesterone C20-oxime in both bases.

FIG. 3B illustrates the degradation of compound 32j (RGF-PROG-104) in human plasma as well as the simultaneous formation of progesterone C20-oxime in both bases.

FIG. 4 illustrates the % increase in tissue water content (edema level) in rat brain 24 h post-TBI. Columns represent mean±SD. All compounds were administered via IM injections 1 h and 6 h post-TBI. At 24 h, the animals were sacrificed, and brain sections were removed for edema assay.

FIG. 5A illustrates the rat plasma pharmacokinetic profile of 32j (RGF-PROG-104) for the IV route in rats. Male rats (n=3 per group) were administered a single dose of prodrug: IV (2 mg/kg). Plasma levels of prodrug 32j and parent progesterone C20-oxime were quantified using LC-MS/MS. The plots show the time-dependent plasma levels of prodrug 32j and parent progesterone C20-oxime.

FIG. 5B illustrates the rat plasma pharmacokinetic profile of 32j (RGF-PROG-104) for the IM route in rats. Male rats (n=3 per group) were administered a single dose of prodrug: IM (10 mg/kg). Plasma levels of prodrug 32j and parent progesterone C20-oxime were quantified using LC-MS/MS. The plots show the time-dependent plasma levels of prodrug 32j and parent progesterone C20-oxime.

FIG. 6A illustrates the rat plasma pharmacokinetic profile of EIDD-1723 and parent progesterone C20-oxime for the IV route in rats. Male rats (n=3 per group) were administered a single dose of prodrug: IV (2 mg/kg). Plasma levels of prodrug EIDD-1723 and parent progesterone C20-oxime were quantified using LC-MS/MS. The plots show the time-dependent plasma levels of prodrug EIDD-1723 and parent progesterone C20-oxime.

FIG. 6B illustrates the rat plasma pharmacokinetic profile of EIDD-1723 and parent progesterone C20-oxime for the IM route in rats. Male rats (n=3 per group) were administered a single dose of prodrug: IM (10 mg/kg). Plasma levels of prodrug EIDD-1723 and parent progesterone C20-oxime were quantified using LC-MS/MS. The plots show the time-dependent plasma levels of prodrug EIDD-1723 and parent progesterone C20-oxime.

### DETAILED DESCRIPTION

The present disclosure describes prodrugs of neurosteroid analogs. In general, the neurosteroid analogs have a 20-carbon skeleton, as shown in Formula II. It also describes pharmaceutical formulations of the prodrugs and methods for treating conditions, disorders, or diseases using the prodrugs.

Generally, the prodrugs have a higher solubility in an aqueous medium than their corresponding neurosteroid analogs. In some cases, the prodrugs may be capable of self-immolative cleavage in response to environmental pH changes, releasing the neurosteroid analogs. In some forms, the prodrugs are stable in an acidic aqueous medium but exhibit a wide range of release kinetics in human plasma. Pharmaceutical formulations containing the prodrugs are also disclosed. Additionally, methods of treating a condition, disorder, or disease using the prodrugs or their pharmaceutical formulations are disclosed. Exemplary conditions, disorders, and diseases relevant to this disclosure include stroke, subarachnoid hemorrhage, cerebral ischemia, cerebral vasospasm, hypoxia, CNS injury, concussion, traumatic brain injury, depression, postpartum depression, epilepsy, seizure disorder, and neurodegenerative disease.

Before the present disclosure is described in greater detail, it is to be understood that this disclosure is not limited to the particular embodiments described herein, and as such, may vary in accordance with the scope of the present disclosure. Unless defined otherwise, all technical and scientific terms used herein have the same meaning as commonly understood by one of ordinary skill in the art to which this disclosure belongs.

All publications and patents cited in this specification are herein incorporated by reference as if each individual publication and patent were specifically and individually indicated to be incorporated by reference. They are incorporated by reference to disclose and describe the methods and/or materials in connection with which the publications and patents are cited.

As will be apparent to those of ordinary skill in the art upon reading this disclosure, each of the particular embodiments described and illustrated herein has discrete components and/or features that may be readily separated from or combined with one or more components and/or features of any of the other embodiments described herein, without departing from the scope or spirit of the present disclosure. Any recited method can be carried out in the order of events recited herein or in any other order that is logically possible.

Embodiments of the present disclosure will employ, unless otherwise indicated, techniques of medicine, organic chemistry, medicinal chemistry, biochemistry, molecular biology, pharmacology, neurology, and the like, which are within the skill of the art. Such techniques are explained fully in the literature, such as the publications and patents cited herein.

### I. DEFINITIONS

As used herein, the singular forms “a,” “an,” and “the” include plural referents unless the context clearly dictates otherwise.

The terms “may,” “may be,” “can,” and “can be,” and related terms are intended to convey that the subject matter involved is optional (that is, the subject matter is present in some examples and is not present in other examples), not a reference to a capability of the subject matter or to a probability, unless the context clearly indicates otherwise.

The terms “optional” and “optionally” mean that the subsequently described event, circumstance, or material may or may not occur or be present, and that the description includes instances where the event, circumstance, or material occurs or is present as well as instances where it does not occur or is not present.

Use of the term “about” is intended to describe values either above or below the stated value in a range of approx. +/−10%; in other examples the values may range in value either above or below the stated value in a range of approx. +/−5%; in other examples the values may range in value either above or below the stated value in a range of approx. +/−2%; in other examples the values may range in value either above or below the stated value in a range of approx. +/−1%.

A carbon range (e.g., C₁-C₁₀) is intended to disclose individually every possible carbon value and/or sub-range encompassed within. For example, a carbon range of C₁-C₁₀ discloses C₁, C₂, C₃, C₄, C₅, C₆, C₇, C₈, C₉, and C₁₀, as well as sub-ranges encompassed therein, such as C₂-C₉, C₃-C₈, C₁-C₅, etc.

As used herein, the term “subject” refers to an animal, including human and non-human animals. Human subjects may include pediatric patients and adult patients. Non-human animals may include domestic pets, livestock and farm animals, and zoo animals. In some cases, the non-human animals may be non-human primates.

As used herein, the terms “prevent” and “preventing” include the prevention of the occurrence, onset, spread, and/or recurrence. It is not intended that the present disclosure is limited to complete prevention. For example, prevention is considered as achieved when the occurrence is delayed, the severity of the onset is reduced, or both.

As used herein, the terms “treat” and “treating” include medical management of a condition, disorder, or disease of a subject as would be understood by a person of ordinary skill in the art (see, for example, Stedman's Medical Dictionary). In general, treatment is not limited to cases where the subject is cured and the condition, disorder, or disease is eradicated. Rather, treatment also contemplates cases where a treatment regimen containing one of the compounds, compositions, or pharmaceutical formulations of the present disclosure provides an improved clinical outcome. The improved clinical outcome may include one or more of the following: abatement, lessening, and/or alleviation of one or more symptoms that result from or are associated with the condition, disorder, or disease to be treated; decreased occurrence of one or more symptoms; improved quality of life; diminishment of the extent of the condition, disorder, or disease; reaching or establishing a stabilized state (i.e., not worsening) of the condition, disorder, or disease; delay or slowing of the progression of the condition, disorder, or disease; amelioration or palliation of the state of the condition, disorder, or disease; partial or total remission; and improvement in survival (whether increase in the overall survival rate or prolonging of survival when compared to expected survival if the subject were not receiving the treatment). For example, the disclosure encompasses treatment that reduces one or more symptoms of and/or cognitive deficit associated with or caused by a brain injury.

The terms “derivative” and “derivatives” refer to chemical compounds/moieties with a structure similar to that of a parent compound/moiety but different from it in respect to one or more components, functional groups, atoms, etc. Optionally, the derivatives retain certain functional attributes of the parent compound/moiety. Optionally, the derivatives can be formed from the parent compound/moiety by chemical reaction(s). The differences between the derivatives and the parent compound/moiety can include, but are not limited to, replacement of one or more functional groups with one or more different functional groups or introducing or removing one or more substituents of hydrogen atoms.

The term “alkyl” refers to univalent groups derived from alkanes (i.e., acyclic saturated hydrocarbons) by removal of a hydrogen atom from any carbon atom. Alkyl groups can be linear or branched. Suitable alkyl groups can have one to 30 carbon atoms, i.e., C₁-C₃₀ alkyl. If the alkyl is branched, it is understood that at least three carbon atoms are present.

The term “alkenyl” refers to univalent groups derived from alkenes by removal of a hydrogen atom from any carbon atom. Alkenes are unsaturated hydrocarbons that contain at least one carbon-carbon double bond. Alkenyl groups can be linear or branched. Suitable alkenyl groups can have two to 30 carbon atoms, i.e., C₂-C₃₀ alkenyl. If the alkenyl is branched, it is understood that at least three carbon atoms are present.

The term “alkynyl” refers to univalent groups derived from alkynes by removal of a hydrogen atom from any carbon atom. Alkynes are unsaturated hydrocarbons that contain at least one carbon-carbon triple bond. Alkynyl groups can be linear or branched. Suitable alkynyl groups can have two to 30 carbon atoms, i.e., C₂-C₃₀ alkynyl. If the alkynyl is branched, it is understood that at least four carbon atoms are present.

The term “heteroalkyl” refers to alkyl groups where one or more carbon atoms are replaced with a heteroatom such as, O, N, S, or Si. Optionally, the nitrogen and/or sulphur heteroatom(s) may be oxidized, and the nitrogen heteroatom(s) may be quaternized. Heteroalkyl groups can be linear or branched. Suitable heteroalkyl groups can have one to 30 carbon atoms, i.e., C₁-C₃₀ heteroalkyl. If the heteroalkyl is branched, it is understood that at least one carbon atom and at least one heteroatom are present.

The term “aryl” refers to univalent groups derived from arenes by removal of a hydrogen atom from a ring atom. Arenes are monocyclic or polycyclic aromatic hydrocarbons. In polycyclic arenes, the rings can be attached together in a pendant manner, a fused manner, or a combination thereof. Accordingly, in polycyclic aryl groups, the rings can be attached together in a pendant manner, a fused manner, or a combination thereof. Suitable aryl groups can have six to 30 carbon atoms, i.e., C₆-C₃₀ aryl. The number of “members” of an aryl group refers to the total number of carbon atoms in the ring(s) of the aryl group.

The term “heteroaryl” refers to univalent groups derived from heteroarenes by removal of a hydrogen atom from a ring atom. Heteroarenes are heterocyclic compounds derived from arenes by replacement of one or more methine (—C═) and/or vinylene (—CH═CH—) groups by trivalent or divalent heteroatoms, respectively, in such a way as to maintain the continuous π-electron system characteristic of aromatic systems and a number of out-of-plane π-electrons corresponding to the Hückel rule (4n+2). Heteroarenes can be monocyclic or polycyclic. In polycyclic heteroarenes, the rings can be attached together in a pendant manner, a fused manner, or a combination thereof. Accordingly, in polycyclic heteroaryl groups, the rings can be attached together in a pendant manner, a fused manner, or a combination thereof. Suitable heteroaryl groups can have one to 30 carbon atoms, i.e., C₁-C₃₀ heteroaryl. The number of “members” of a heteroaryl group refers to the total number of carbon atom(s) and heteroatom(s) in the ring(s) of the heteroaryl group.

“Carbocycle” or “carbocyclyl” refers to mono- and polycyclic ring systems containing only carbon atoms as ring atoms. The mono- and polycyclic ring systems may be aromatic, non-aromatic (saturated or unsaturated), or a mixture of aromatic and non-aromatic rings. Carbocyclyls are univalent, derived from carbocycles by removal of a hydrogen atom from a ring atom. Carbocycles include arenes; carbocyclyls include aryls. In polycyclic carbocycles or carbocyclyls, the rings can be attached together in a pendant manner (i.e., two rings are connected by a single bond), a spiro manner (i.e., two rings are connected through a defining single common atom), a fused manner (i.e., two rings share two adjacent atoms; in other words, two rings share one covalent bond), a bridged manner (i.e., two rings share three or more atoms, separating the two bridgehead atoms by a bridge containing at least one atom), or a combination thereof. Suitable carbocycle or carbocyclyl groups can have three to 30 carbon atoms, i.e., C₃-C₃₀ carbocycle or carbocyclyl. The number of “members” of a carbocycle or carbocyclyl group refers to the total number of carbon atoms in the ring(s) of the carbocycle or carbocyclyl group.

“Heterocycle” or “heterocyclyl” refers to mono- and polycyclic ring systems containing at least one carbon atom and one or more heteroatoms independently selected from elements like nitrogen, oxygen, and sulfur, as ring atoms. Optionally, the nitrogen and/or sulphur heteroatom(s) may be oxidized, and the nitrogen heteroatom(s) may be quaternized. The mono- and polycyclic ring systems may be aromatic, non-aromatic, or a mixture of aromatic and non-aromatic rings. Heterocyclyls are univalent, derived from heterocycles by removal of a hydrogen atom from a ring atom. Heterocycles include heteroarenes; heterocyclyls include heteroaryls. In polycyclic heterocycle or heterocyclyl groups, the rings can be attached together in a pendant manner (i.e., two rings are connected by a single bond), a spiro manner (i.e., two rings are connected through a defining single common atom), a fused manner (i.e., two rings share two adjacent atoms; in other words, two rings share one covalent bond), a bridged manner (i.e., two rings share three or more atoms, separating the two bridgehead atoms by a bridge containing at least one atom), or a combination thereof. Suitable heterocycle or heterocyclyl groups can have one to 30 carbon atoms, i.e., C₁-C₃₀ heterocycle or heterocyclyl. The number of “members” of a heterocycle or heterocyclyl group refers to the total number of carbon atom(s) and heteroatom(s) in the ring(s) of the heterocycle or heterocyclyl group.

As used herein, the terms “halogen” and “halo” refer to fluorine, chlorine, bromine, and iodine.

As used herein, “haloalkyl” refers to halogen-substituted alkyl groups. Optionally, the haloalkyl groups contain one halogen substituent. Optionally, the haloalkyl groups contain multiple halogen substituents, i.e., polyhaloalkyl. In some examples, the haloalkyl groups contain one or more fluorine substituents.

As used herein, “haloalkenyl” refers to halogen-substituted alkenyl groups. Optionally, the haloalkenyl groups contain one halogen substituent. Optionally, the haloalkenyl groups contain multiple halogen substituents. In some examples, the haloalkenyl groups contain one or more fluorine substituents.

As used herein, “haloalkynyl” refers to halogen-substituted alkynyl groups. Optionally, the haloalkynyl groups contain one halogen substituent. Optionally, the haloalkynyl groups contain multiple halogen substituents. In some examples, the haloalkynyl groups contain one or more fluorine substituents.

As used herein, “halocarbocyclyl” refers to halogen-substituted carbocyclyl groups. Optionally, the halocarbocyclyl groups contain one halogen substituent. Optionally, the halocarbocyclyl groups contain multiple halogen substituents. In some examples, the halocarbocyclyl groups contain one or more fluorine substituents.

As used herein, “haloheterocyclyl” refers to halogen-substituted heterocyclyl groups. Optionally, the haloheterocyclyl groups contain one halogen substituent. Optionally, the haloheterocyclyl groups contain multiple halogen substituents. In some examples, the haloheterocyclyl groups contain one or more fluorine substituents.

As used herein, “haloaryl” refers to halogen-substituted aryl groups. Optionally, the haloaryl groups contain one halogen substituent. Optionally, the haloaryl groups contain multiple halogen substituents. In some examples, the haloaryl groups contain one or more fluorine substituents.

As used herein, “haloheteroaryl” refers to halogen-substituted heteroaryl groups. Optionally, the haloheteroaryl groups contain one halogen substituent. Optionally, the haloheteroaryl groups contain multiple halogen substituents. In some examples, the haloheteroaryl groups contain one or more fluorine substituents.

The term “substituted,” as used herein, means that the chemical group or moiety contains one or more substituents replacing the hydrogen atom(s) in the original chemical group or moiety. It is understood that any substitution is in accordance with a permitted valence of the substituted atom and the substituent and that the substitution results in a stable compound, e.g., a compound that does not spontaneously undergo transformation such as by rearrangement, cyclization, elimination, etc., under room temperature. Unless otherwise specified, the substituents are R groups. The R groups, on each occurrence, can be independently selected from halogen, alkyl, haloalkyl, alkenyl, haloalkenyl, alkynyl, haloalkynyl, carbocyclyl, halocarbocyclyl, heterocyclyl, haloheterocyclyl, aryl, haloaryl, heteroaryl, haloheteroaryl, —OH, —SH, —NH₂, —N₃, —OCN, —NCO, —ONO₂, —CN, —NC, —ONO, —CONH₂, —NO, —NO₂, —ONH₂, —SCN, —SNCS, —CF₃, —CH₂CF₃, —CH₂Cl, —CHCl₂, —CH₂NH₂, —NHCOH, —CHO, —COOH, —SO₃H, —CH₂SO₂CH₃, —PO₃H₂, —OPO₃H₂, —P(═O)(OR^{G1})(OR^{G2}), —OP(═O)(OR^{G1})(OR^{G2}), —BR^{G1}(OR^{G2}), —B(OR^{G1})(OR^{G2}), —Si(R^{G1})(R^{G2})(R^{G3}), C(R^{G1})(R^{G2})(R^{G3}), —N[(R^{G1})(R^{G2})(R^{G3})]⁺, and —GR^{G1}, in which -G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G3}—, —C(═S)—, —C(═S)S—, —SC(═S)—, —SC(═S)S—, —C(═NR^{G2})—, —C(═NR^{G2})O—, —C(═NR^{G2}) NR^{G3}—, —OC(═NR^{G2})—, —NR^{G2}C(═NR^{G3})—, —NR^{G2}SO₂—, —C(═NR^{G2}) NR^{G3}—, —OC(═NR^{G2})—, —NR^{G2}C(═NR^{G3})—, —NR^{G2}SO₂—, —NR^{G2}SO₂NR^{G3}—, —NR^{G2}C(═S)—, —SC(═S)NR^{G2}—, —NR^{G2}C(═S)S—, —NR^{G2}C(═S)NR^{G3}—, —SC(═NR^{G2})—, —C(═S)NR^{G2}—, —OC(═S)NR^{G2}—, —NR^{G2}C(═S)O—, —SC(═O)NR^{G2}—, —NR^{G2}C(═O)S—, —C(═O)S—, —SC(═O)—, —SC(═O)S—, —C(═S)O—, —OC(═S)—, —OC(═S)O—, —SO₂NR^{G2}—, —BR^{G2}—, or —PR^{G2}—, wherein each occurrence of R^{G1}, R^{G2}, and R^{G3} is independently selected from hydrogen, halogen, alkyl, haloalkyl, alkenyl, haloalkenyl, alkynyl, haloalkynyl, carbocyclyl, halocarbocyclyl, heterocyclyl, haloheterocyclyl, aryl, haloaryl, heteroaryl, and haloheteroaryl. Optionally, two R groups on the same atom can join together with that atom to form a cyclic moiety, such as a carbocycle or a heterocycle. Alternatively, two R groups on the same atom can merge into one oxygen (═O) or sulfur (═S) atom.

The term “optionally substituted,” as used herein, means that substitution is optional, and therefore it is possible for the designated atom/chemical group/compound to be unsubstituted.

As used herein, “ester” refers to —C(═O)OR^{c1} or —OC(═O)R^{c2}, wherein R^{c1} and R^{c2} are independently selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same atom can join together with that atom to form a cyclic moiety, such as a carbocycle or a heterocycle.

As used herein, “amino” refers to —NR^{d1}R^{d2}, wherein R^{d1} and R^{d2} are independently selected from hydrogen, alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same atom can join together with that atom to form a cyclic moiety, such as a carbocycle or a heterocycle. When R^{d1} and R^{d2} are each hydrogen, the amino group is a primary amino group.

As used herein, “acyl” refers —C(═O)R^{e}, wherein R^{e} is selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally substituted by one or more R groups described above. Optionally, two R groups on the same atom can join together with that atom to form a cyclic moiety, such as a carbocycle or a heterocycle.

As used herein, “amide” refers to —C(═O)NR^{f1}R^{f2}, wherein R^{f1} and R^{f2} are independently selected from hydrogen, alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same atom can join together with that atom to form a cyclic moiety, such as a carbocycle or a heterocycle. When R^{f1} and R^{f2} are each hydrogen, the amide group is a carbamoyl group.

As used herein, “carbonate ester” refers to —OC(═O)ORⁱ, wherein Rⁱ is selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally substituted by one or more R groups described above. Optionally, two R groups on the same atom can join together with that atom to form a cyclic moiety, such as a carbocycle or a heterocycle.

As used herein, “carbamate” refers to —OC(═O)NR^{j1}R^{j2} or —NR^{k}[(C═O)OR^{l}], wherein R^{j1}, R^{j2}, R^{k}, and R^{l} are independently selected from hydrogen, alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same atom can join together with that atom to form a cyclic moiety, such as a carbocycle or a heterocycle.

As used herein, “sulfinyl” refers to —S(═O)R^{m}), wherein R^{m} is selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally substituted by one or more R groups described above. Optionally, two R groups on the same atom can join together with that atom to form a cyclic moiety, such as a carbocycle or a heterocycle.

As used herein, “sulfonyl” refers to —S(═O)₂Rⁿ, wherein Rⁿ is selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally substituted by one or more R groups described above. Optionally, two R groups on the same atom can join together with that atom to form a cyclic moiety, such as a carbocycle or a heterocycle.

As used herein, “thioester” refers to —C(═O) SR^{o1} or —SC(═O)R^{o2}, wherein R^{o1} and R^{o2} are independently selected from alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same atom can join together with that atom to form a cyclic moiety, such as a carbocycle or a heterocycle.

As used herein, “sulfonamide” refers to —S(═O)₂NR^{p1}R^{p2}, wherein R^{p1} and R^{p2} are independently selected from hydrogen, alkyl, alkenyl, alkynyl, carbocyclyl, heterocyclyl, aryl, and heteroaryl, wherein each of the alkyl, alkenyl, alkynyl, carbocyclyl, heterocyclyl, aryl, and heteroaryl can be optionally and independently substituted by one or more R groups described above. Optionally, two R groups on the same atom can join together with that atom to form a cyclic moiety, such as a carbocycle or a heterocycle. When R^{p1} and R^{p2} are each hydrogen, the amide group is a sulfamoyl group.

As used herein, “thiol” refers to the univalent radical —SH.

As used herein, “sulfonate” refers to —SO₃⁻.

As used herein, “silyl” refers to the univalent radical derived from silane by removal of a hydrogen atom, i.e., —SiH₃.

As used herein, “carbonate” refers to —O(C═O)OH.

As used herein, the term “stereoisomer” refers to compounds made up of the same atoms having the same bond order but having different three-dimensional arrangements of atoms which are not interchangeable. As used herein, the term “enantiomer” refers to a pair of stereoisomers that are non-superimposable mirror images of one another. As used herein, the term “diastereomer” refers to two stereoisomers that are not mirror images but also not superimposable. The terms “racemate” and “racemic mixture” refer to a mixture of enantiomers. The term “chiral center” refers to a carbon atom to which four different groups are attached. Choice of the appropriate chiral column, eluent, and conditions necessary for effective separation of stereoisomers, such as a pair of enantiomers, is well known to one of ordinary skill in the art (e.g., Jacques et al., Enantiomers, Racemates, and Resolutions, John Wiley and Sons, Inc., 1981).

As used herein, the term “pharmaceutically acceptable” refers to compounds, materials, compositions, or formulations which are, within the scope of sound medical judgment, suitable for use in contact with the tissues of human beings and non-human animals without excessive toxicity, irritation, allergic response, or other problems or complications that commensurate with a reasonable benefit/risk ratio, in accordance with the guidelines of regulatory agencies of a certain country, such as the Food and Drug Administration (FDA) in the United States or its corresponding agencies in countries other than the United States (e.g., the European Medicines Agency (EMA) in Europe, the National Medical Products Administration (NMPA) in China).

As used herein, the term “salt” refers to acid or base salts of the original compound. In some cases, the salt is formed in situ during preparation of the original compound, i.e., the designated synthetic chemistry procedures produce the salt instead of the original compound. In some cases, the salt is obtained via modification of the original compound. In some cases, the salt is obtained via ion exchange with an existing salt of the original compound. Examples of salts include, but are not limited to, mineral or organic acid salts of basic residues such as amines, as well as alkali or organic salts of acidic residues such as carboxylic acids and phosphonic acids. For original compounds containing a basic residue, the salts can be prepared by treating the compounds with an appropriate amount of a non-toxic inorganic or organic acid; alternatively, the salts can be formed in situ during preparation of the original compounds. Exemplary salts of the basic residue include salts with an inorganic acid selected from hydrochloric, hydrobromic, sulfuric, sulfamic, phosphoric, and nitric acids or with an organic acid selected from acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, naphthalenesulfonic, methanesulfonic, ethane disulfonic, oxalic, and isethionic acids. For original compounds containing an acidic residue, the salts can be prepared by treating the compounds with an appropriate amount of a non-toxic base; alternatively, the salts can be formed in situ during preparation of the original compounds. Exemplary salts of the acidic residue include salts with a base selected from ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, and histidine. Optionally, the salts can be prepared by reacting the free acid or base form of the original compounds with a stoichiometric amount or more of an appropriate base or acid, respectively, in water or an aqueous solution, an organic solvent or an organic solution, or a mixture thereof. Lists of exemplary pharmaceutically acceptable salts can be found in Remington's Pharmaceutical Sciences, 20th Ed., Lippincott Williams & Wilkins, Baltimore, MD, 2000 as well as Handbook of Pharmaceutical Salts: Properties, Selection, and Use, Stahl and Wermuth, Eds., Wiley-VCH, Weinheim, 2002.

As used herein, the term “excipient” refers to any components present in the pharmaceutical formulations disclosed herein, other than the active ingredient (i.e., a compound or composition of the present disclosure).

As used herein, the term “effective amount” of a material refers to a nontoxic but sufficient amount of the material to provide the desired result. The exact amount required may vary from subject to subject, depending on the species, age, and general condition of the subject, the severity of the condition, disorder, or disease that is being treated, the active ingredient or therapy used, and the like.

As used herein, the term “physiological pH” refers to the pH that normally prevails in the human body in the absence of pathological states. Typically, it ranges between 7.35 and 7.45.

### II. COMPOUNDS

Disclosed are prodrugs of neurosteroid analogs. In general, the neurosteroid analogs have a 20-carbon skeleton, as shown in Formula II.

To the extent that chemical formulas described herein contain one or more unspecified chiral centers, the formulas are intended to encompass all stable stereoisomers, enantiomers, and diastereomers. Such compounds can exist as a single enantiomer, a racemic mixture, a mixture of diastereomers, or combinations thereof. It is also understood that the chemical formulas encompass all tautomeric forms if tautomerization occurs.

Methods of making exemplary compounds are disclosed in subsequent sections and exemplified by the Examples. The synthetic methods disclosed herein are compatible with a wide variety of functional groups and starting materials. Thus, a wide variety of compounds can be obtained from the disclosed methods.

Optionally, the alkyl groups described herein have 1-30 carbon atoms, i.e., C₁-C₃₀ alkyl. In some forms, the C₁-C₃₀ alkyl can be a linear C₁-C₃₀ alkyl or a branched C₃-C₃₀ alkyl. Optionally, the alkyl groups have 1-20 carbon atoms, i.e., C₁-C₂₀ alkyl. In some forms, the C₁-C₂₀ alkyl can be a linear C₁-C₂₀ alkyl or a branched C₃-C₂₀ alkyl. Optionally, the alkyl groups have 1-10 carbon atoms, i.e., C₁-C₁₀ alkyl. In some forms, the C₁-C₁₀ alkyl can be a linear C₁-C₁₀ alkyl or a branched C₃-C₁₀ alkyl. Optionally, the alkyl groups have 1-6 carbon atoms, i.e., C₁-C₆ alkyl. In some forms, the C₁-C₆ alkyl can be a linear C₁-C₆ alkyl or a branched C₃-C₆ alkyl. Representative straight chain alkyl groups include methyl, ethyl, n-propyl, n-butyl, n-pentyl, n-hexyl, n-heptyl, n-octyl, n-nonyl, and the like. Representative branched alkyl groups include isopropyl, sec-butyl, isobutyl, tert-butyl, isopentyl, and the like.

Optionally, the heteroalkyl groups described herein have 1-30 carbon atoms, i.e., C₁-C₃₀ heteroalkyl. In some forms, the C₁-C₃₀ heteroalkyl can be a linear C₁-C₃₀ heteroalkyl or a branched C₁-C₃₀ heteroalkyl. Optionally, the heteroalkyl groups have 1-20 carbon atoms, i.e., C₁-C₂₀ heteroalkyl. In some forms, the C₁-C₂₀ heteroalkyl can be a linear C₁-C₂₀ heteroalkyl or a branched C₁-C₂₀ heteroalkyl. Optionally, the heteroalkyl groups have 1-10 carbon atoms, i.e., C₁-C₁₀ heteroalkyl. In some forms, the C₁-C₁₀ heteroalkyl can be a linear C₁-C₁₀ heteroalkyl or a branched C₁-C₁₀ heteroalkyl. Optionally, the heteroalkyl groups have 1-6 carbon atoms, i.e., C₁-C₆ heteroalkyl. In some forms, the C₁-C₆ heteroalkyl can be a linear C₁-C₆ heteroalkyl or a branched C₁-C₆ heteroalkyl.

Optionally, the alkenyl groups described herein have 2-30 carbon atoms, i.e., C₂-C₃₀ alkenyl. In some forms, the C₂-C₃₀ alkenyl can be a linear C₂-C₃₀ alkenyl or a branched C₃-C₃₀ alkenyl. Optionally, the alkenyl groups have 2-20 carbon atoms, i.e., C₂-C₂₀ alkenyl. In some forms, the C₂-C₂₀ alkenyl can be a linear C₂-C₂₀ alkenyl or a branched C₃-C₂₀ alkenyl. Optionally, the alkenyl groups have 2-10 carbon atoms, i.e., C₂-C₁₀ alkenyl. In some forms, the C₂-C₁₀ alkenyl can be a linear C₂-C₁₀ alkenyl or a branched C₃-C₁₀ alkenyl. Optionally, the alkenyl groups have 2-6 carbon atoms, i.e., C₂-C₆ alkenyl. In some forms, the C₂-C₆ alkenyl can be a linear C₂-C₆ alkenyl or a branched C₃-C₆ alkenyl. Representative alkenyl groups include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like.

Optionally, the alkynyl groups described herein have 2-30 carbon atoms, i.e., C₂-C₃₀ alkynyl. In some forms, the C₂-C₃₀ alkynyl can be a linear C₂-C₃₀ alkynyl or a branched C₄-C₃₀ alkynyl. Optionally, the alkynyl groups have 2-20 carbon atoms, i.e., C₂-C₂₀ alkynyl. In some forms, the C₂-C₂₀ alkynyl can be a linear C₂-C₂₀ alkynyl or a branched C₄-C₂₀ alkynyl. Optionally, the alkynyl groups have 2-10 carbon atoms, i.e., C₂-C₁₀ alkynyl. In some forms, the C₂-C₁₀ alkynyl can be a linear C₂-C₁₀ alkynyl or a branched C₄-C₁₀ alkynyl. Optionally, the alkynyl groups have 2-6 carbon atoms, i.e., C₂-C₆ alkynyl. In some forms, the C₂-C₆ alkynyl can be a linear C₂-C₆ alkynyl or a branched C₄-C₆ alkynyl. Representative alkynyl groups include ethynyl, propynyl, 1-butynyl, 2-butynyl, 1-pentynyl, 2-pentynyl, 3-methyl-1-butynyl, and the like.

Optionally, the aryl groups described herein have 6-30 carbon atoms, i.e., C₆-C₃₀ aryl. Optionally, the aryl groups have 6-20 carbon atoms, i.e., C₆-C₂₀ aryl. Optionally, the aryl groups have 6-12 carbon atoms, i.e., C₆-C₁₂ aryl. Representative aryl groups include phenyl, naphthyl, and biphenyl.

Optionally, the heteroaryl groups described herein have 1-30 carbon atoms, i.e., C₁-C₃₀ heteroaryl. Optionally, the heteroaryl groups have 1-20 carbon atoms, i.e., C₁-C₂₀ heteroaryl. Optionally, the heteroaryl groups have 1-11 carbon atoms, i.e., C₁-C₁₁ heteroaryl. Optionally, the heteroaryl groups have 1-5 carbon atoms, i.e., C₁-C₅ heteroaryl. Optionally, the heteroaryl groups are 5-20 membered heteroaryl groups. Optionally, the heteroaryl groups are 5-12 membered heteroaryl groups. Optionally, the heteroaryl groups are 5 or 6 membered heteroaryl groups. Representative heteroaryl groups include furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl.

Optionally, the carbocyclyl groups described herein have 3-30 carbon atoms, i.e., C₃-C₃₀ carbocyclyl. Optionally, the carbocyclyl groups described herein have 3-20 carbon atoms, i.e., C₃-C₂₀ carbocyclyl. Optionally, the carbocyclyl groups described herein have 3-12 carbon atoms, i.e., C₃-C₁₂ carbocyclyl. Representative saturated carbocyclyl groups include cyclopropyl, cyclobutyl, cyclopentyl, cyclohexyl, and the like. Representative unsaturated carbocyclyl groups include cyclopentenyl, cyclohexenyl, and the like.

Optionally, the heterocyclyl groups described herein have 1-30 carbon atoms, i.e., C₁-C₃₀ heterocyclyl. Optionally, the heterocyclyl groups described herein have 1-20 carbon atoms, i.e., C₁-C₂₀ heterocyclyl. Optionally, the heterocyclyl groups described herein have 1-11 carbon atoms, i.e., C₁-C₁₁ heterocyclyl. Optionally, the heterocyclyl groups described herein have 1-6 carbon atoms, i.e., C₁-C₆ heterocyclyl. Optionally, the heterocyclyl groups are 3-20 membered heterocyclyl groups. Optionally, the heterocyclyl groups are 3-12 membered heterocyclyl groups. Optionally, the heteroaryl groups are 4-7 membered heterocyclyl groups.

The optionally substituted groups described in the chemical formulas described herein (e.g., Formula II and its sub-formulas), on each occurrence when not specified, may have one or more substituents in the form of the R groups described above.

The R groups, on each occurrence, can be independently selected from halogen, alkyl, haloalkyl, alkenyl, haloalkenyl, alkynyl, haloalkynyl, carbocyclyl, halocarbocyclyl, heterocyclyl, haloheterocyclyl, aryl, haloaryl, heteroaryl, haloheteroaryl, —OH, —SH, —NH₂, —N₃, —OCN, —NCO, —ONO₂, —CN, —NC, —ONO, —CONH₂, —NO, —NO₂, —ONH₂, —SCN, —SNCS, —CF₃, —CH₂CF₃, —CH₂Cl, —CHCl₂, CH₂NH₂, —NHCOH, —CHO, —COOH, —SO₃H, —CH₂SO₂CH₃, —PO₃H₂, —OPO₃H₂, —P(═O)(OR^{G1})(OR^{G2}), —OP(═O)(OR^{G1})(OR^{G2}), —BR^{G1} (OR^{G2}), —B(OR^{G1})(OR^{G2}), —Si(R^{G1})(R^{G2})(R^{G3}), —C(R^{G1})(R^{G2})(R^{G3}), —N[(R^{G1})(R^{G2})(R^{G3})]⁺, and —GR^{G1}, in which -G is —O—, —S—, —NR^{G2}—, —C(═O)—, —S(═O)—, —SO₂—, —C(═O)O—, —C(═O)NR^{G2}—, —OC(═O)—, —NR^{G2}C(═O)—, —OC(═O)O—, —OC(═O)NR^{G2}—, —NR^{G2}C(═O)O—, —NR^{G2}C(═O)NR^{G3}—, —C(═S)—, —C(═S)S—, —SC(═S)—, —SC(═S)S—, —C(═NR^{G2})—, —C(═NR^{G2})O—, —C(═NR^{G2}) NR^{G3}—, —OC(═NR^{G2})—, —NR^{G2}C(═NR^{G3})—, —NR^{G2}SO₂—, —C(═NR^{G2}) NR^{G3}—, —OC(═NR^{G2})—, —NR^{G2}C(═NR^{G3})—, —NR^{G2}SO₂—, —NR^{G2}SO₂NR^{G3}—, —NR^{G2}C(═S)—, —SC(═S)NR^{G2}—, NR^{G2}C(═S)S—, —NR^{G2}C(═S)NR^{G3}—, —SC(═NR^{G2})—, —C(═S)NR^{G2}—, —OC(═S)NR^{G2}—, —NR^{G2}C(═S)O—, —SC(═O)NR^{G2}—, —NR^{G2}C(═O)S—, —C(═O)S—, —SC(═O)—, —SC(═O)S—, —C(═S)O—, —OC(═S)—, —OC(═S)O—, —SO₂NR^{G2}—, —BR^{G2}—, or —PR^{G2}—, wherein each occurrence of R^{G1}, R^{G2}, and R^{G3} is independently selected from hydrogen, halogen, alkyl, haloalkyl, alkenyl, haloalkenyl, alkynyl, haloalkynyl, carbocyclyl, halocarbocyclyl, heterocyclyl, haloheterocyclyl, aryl, haloaryl, heteroaryl, and haloheteroaryl. Optionally, two R groups on the same atom can join together with that atom to form a cyclic moiety, such as a carbocycle or a heterocycle. Alternatively, two R groups on the same atom can merge into one oxygen (═O) or sulfur (═S) atom.

In some examples, the R groups are independently selected from halogen, nitro, cyano, hydroxyl, formyl, carboxyl, thiol, —O (counting as two R groups), ═S (counting as two R groups), sulfamoyl, alkyl (such as methyl, ethyl, isopropyl, tert-butyl), haloalkyl (such as trifluoromethyl), alkenyl, haloalkenyl, alkynyl, haloalkynyl, carbocyclyl, halocarbocyclyl, heterocyclyl, haloheterocyclyl, aryl, haloaryl, heteroaryl, haloheteroaryl, arylalkyl (such as benzyl), alkylaryl, alkyloxy (such as methoxy, ethoxy), haloalkyloxy (such as trifluoromethoxy), aryloxy, alkylcarbonyl (such as acetyl), arylcarbonyl (such as benzoyl), alkylcarbonyloxy (such as acetoxy), arylcarbonyloxy (such as benzoyloxy), alkyloxycarbonyl (such as methoxycarbonyl, ethoxycarbonyl, isopropoxycarbonyl), aryloxycarbonyl, primary amino, alkylamino (such as methylamino, ethylamino, dimethylamino, diethylamino, N-methyl-N-ethylamino), alkylammonium (such as trimethylammonium), alkylcarbonylamino (such as acetylamino), arylcarbonylamino (such as benzoylamino), carbamoyl, N-alkylcarbamoyl (such as N-methylcarbamoyl, N-ethylcarbamoyl, N,N-dimethylcarbamoyl, N,N-diethylcarbamoyl, N-methyl-N-ethylcarbamoyl), alkylthio (such as methylthio, ethylthio), alkylsulfinyl (such as methylsulfinyl, ethylsulfinyl), alkylsulfonyl (such as mesyl, ethylsulfonyl), and N-alkylsulfamoyl (such as N-methylsulfamoyl, N-ethylsulfamoyl, N,N-dimethylsulfamoyl, N,N-diethylsulfamoyl, N-methyl-N-ethylsulfamoyl).

In some examples, the R groups are independently selected from halogen, nitro, cyano, hydroxyl, trifluoromethyl, methoxy, ethoxy, trifluoromethoxy, primary amino, formyl, carboxyl, carbamoyl, thiol, ═O, ═S, sulfamoyl, acetyl, acetoxy, methoxycarbonyl, ethoxycarbonyl, isopropoxycarbonyl, methylamino, ethylamino, dimethylamino, diethylamino, N-methyl-N-ethylamino, trimethylammonium, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, N-methylsulfamoyl, N-ethylsulfamoyl, N,N-dimethylsulfamoyl, N,N-diethylsulfamoyl, N-methyl-N-ethylsulfamoyl, benzyl, benzoyl, alkyl, haloalkyl, alkenyl, haloalkenyl, alkynyl, haloalkynyl, carbocyclyl, halocarbocyclyl, heterocyclyl, haloheterocyclyl, aryl, haloaryl, heteroaryl, and haloheteroaryl.

In some examples, the R groups are independently selected from halogen, ═O, ═S, alkyl, haloalkyl, carbocyclyl, halocarbocyclyl, aryl, haloaryl, heterocyclyl, and haloheterocyclyl.

As used herein, “alkyloxy” refers to a hydroxyl group substituted by an alkyl group at the oxygen atom. Exemplary alkyloxy groups include, but are not limited to, methoxy, ethoxy, n-propoxy, i-propoxy, n-butoxy, s-butoxy, 1-butoxy, n-pentoxy, and s-pentoxy.

As used herein, “haloalkyloxy” refers to a hydroxyl group substituted by a haloalkyl group at the oxygen atom. An example of haloalkyloxy is trifluoromethoxy.

As used herein, “aryloxy” refers to a hydroxyl group substituted by an aryl group at the oxygen atom.

As used herein, “alkylcarbonyl” refers to an alkyl group attached through a carbonyl bridge (—C(═O)—).

As used herein, “arylcarbonyl” refers to an aryl group attached through a carbonyl bridge.

As used herein, “alkylcarbonyloxy” refers to a hydroxyl group substituted by an alkylcarbonyl group at the oxygen atom of the hydroxyl group.

As used herein, “arylcarbonyloxy” refers to a hydroxyl group substituted by an arylcarbonyl group at the oxygen atom of the hydroxyl group.

As used herein, “alkyloxycarbonyl” refers to an alkyloxy group attached through a carbonyl bridge.

As used herein, “aryloxycarbonyl” refers to an aryloxy group attached through a carbonyl bridge.

As used herein, “alkylamino” refers to a primary amino group substituted by one or two alkyl groups. When the primary amino group is substituted by two alkyl groups, the two alkyl groups can be the same or different. An example of alkylamino is methylamino (i.e., —NH—CH₃).

As used herein, “alkylammonium” refers to a primary ammonium group substituted by one, two, or three alkyl groups. When the primary ammonium group is substituted by two or three alkyl groups, the two or three alkyl groups can be the same or different. An example of alkylammonium is trimethylammonium (i.e., —N(CH₃)₃⁺).

As used herein, “alkylcarbonylamino” refers to a primary amino group substituted by one alkylcarbonyl group.

As used herein, “arylcarbonylamino” refers to a primary amino group substituted by one arylcarbonyl group.

As used herein, “N-alkylcarbamoyl” refers to a carbamoyl group (—C(═O)—NH₂) substituted by one or two alkyl groups at the nitrogen atom. When the carbamoyl group is substituted by two alkyl groups, the two alkyl groups can be the same or different.

As used herein, “alkylthio” refers to a thiol group substituted by an alkyl group at the sulfur atom. An example of alkylthio is methylthio (i.e., —S—CH₃).

As used herein, “alkylsulfinyl” refers to an alkyl group attached through a sulfinyl bridge (—S(═O)—).

As used herein, “alkylsulfonyl” refers to an alkyl group attached through a sulfonyl bridge (—S(═O)₂—).

As used herein, “N-alkylsulfamoyl” refers to a sulfamoyl group (—S(═O)₂—NH₂) substituted by one or two alkyl groups at the nitrogen atom. When the sulfamoyl group is substituted by two alkyl groups, the two alkyl groups can be the same or different.

### A. General Structure

In some embodiments, the compounds have a structure of Formula II or a pharmaceutically acceptable salt, hydrate, or hydrated salt of Formula II,

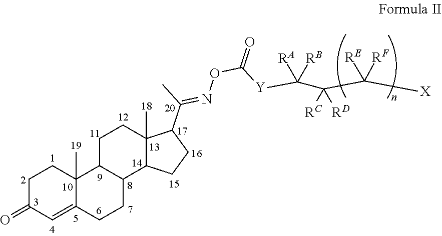

wherein n is 0 or 1; and
wherein:
(1) X is OH or NR 1R²,
Y is O or NR³,
R^{A}, R^{B}, R^{C}, R^{D}, R^{E}, and R^{F} are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R¹, R², and R³ are independently selected from hydrogen, optionally substituted alkyl, optionally substituted heteroalkyl, optionally substituted carbocyclyl, optionally substituted heterocyclyl, optionally substituted aryl, and optionally substituted heteroaryl, with the proviso that at least one of R¹ and R² is hydrogen;
(2) X is NR¹R²,
Y is O or NR³,
R¹ joins R^{C} or R^{E} to form a 4-7 membered, optionally substituted heterocycle,
R² is hydrogen,
R^{A}, R^{B}, R^{C}, R^{D}, R^{E}, and R^{F}, on each occurrence when not joined by R¹ to form the heterocycle, are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R³ is independently selected from hydrogen, optionally substituted alkyl, optionally substituted heteroalkyl, optionally substituted carbocyclyl, optionally substituted heterocyclyl, optionally substituted aryl, and optionally substituted heteroaryl; or
(3) X is OH or NR¹R²,
Y is NR³,
R³ joins R^{A} to form a 4-7 membered, optionally substituted heterocycle,
R^{B}, R^{C}, R^{D}, R^{E}, and R^{F} are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R¹ and R² are independently selected from hydrogen, optionally substituted alkyl, optionally substituted heteroalkyl, optionally substituted carbocyclyl, optionally substituted heterocyclyl, optionally substituted aryl, and optionally substituted heteroaryl, with the proviso that at least one of R¹ and R² is hydrogen.

The numberings of carbon atoms in Formula II apply to all sub-formulas of Formula II, including Formulas IIA, IIB, IIC, IID, II-1, II-1A, II-1B, II-1C, and II-1D.

In some embodiments, the compounds are in a non-salt form as shown in Formula II. In some embodiments, the compounds are in a salt form. In some embodiments, the compounds are in an HCl salt form.

In some embodiments, n is 0, and Formula II is in the form of Formula IIA.

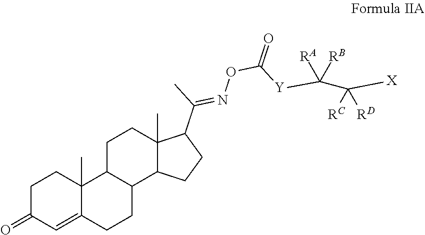

In some embodiments, n is 1, and Formula II is in the form of Formula IIB.

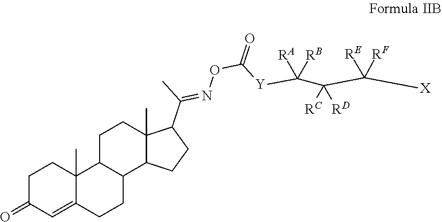

Substituents at the C20-oxime moiety

### Group I

In some embodiments, the compounds have the following features:
X is OH or NR¹R²,
Y is O or NR³,
R^{A}, R^{B}, R^{C}, R^{D}, R^{E}, and R^{F} are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R¹, R², and R³ are independently selected from hydrogen, optionally substituted alkyl, optionally substituted heteroalkyl, optionally substituted carbocyclyl, optionally substituted heterocyclyl, optionally substituted aryl, and optionally substituted heteroaryl, with the proviso that at least one of R¹ and R² is hydrogen.

In some embodiments, X is OH. In some embodiments, X is NR¹R². In some embodiments, both R¹ and R² are hydrogen. In some embodiments, R¹ is optionally substituted alkyl, and R² is hydrogen. In some embodiments, R¹ is optionally substituted C₁-C₄ alkyl, and R² is hydrogen. In some embodiments, R¹ is selected from —CH₃, —CF₃, —CH₂CH₃, —CH₂CH₂OH, CH₂CH₂F, CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R² is hydrogen. In some embodiments, R¹ is —CH₃, and R² is hydrogen. In some embodiments, R¹ is —CF₃, and R² is hydrogen. In some embodiments, R¹ is —CH₂CH₃, and R² is hydrogen. In some embodiments, R¹ is —CH₂CH₂OH, and R² is hydrogen. In some embodiments, R¹ is —CH₂CH₂F, and R² is hydrogen. In some embodiments, R¹ is —CH₂CF₃, and R² is hydrogen. In some embodiments, R¹ is —CH₂OCF₃, and R² is hydrogen. In some embodiments, R¹ is —CH₂CH₂OCF₃, and R² is hydrogen. In some embodiments, R¹ is —CH(CH₃)₂, and R² is hydrogen. In some embodiments, R¹ is —CH₂COOH, and R² is hydrogen. In some embodiments, R¹ is —CH₂CH₂COOH, and R² is hydrogen. In some embodiments, R¹ is —CH₂CH(CH₃)₂, and R² is hydrogen. In some embodiments, R¹ is —C(CH₃)₃, and R² is hydrogen. In some embodiments, R¹ is —CH₂CH₂NH₂, and R² is hydrogen. In some embodiments, R¹ is —CH₂CH₂NHCH₃, and R² is hydrogen. In some embodiments, R¹ is —CH₂CH₂N(CH₃)₂, and R² is hydrogen. In some embodiments, R¹ is —CH₂CH₂N(CH₃)₃⁺, and R² is hydrogen.

In some embodiments, Y is O. In some embodiments, Y is NR³. In some embodiments, R³ is hydrogen. In some embodiments, R³ is optionally substituted alkyl. In some embodiments, R³ is optionally substituted C₁-C₄ alkyl. In some embodiments, R³ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, R³ is —CH₃. In some embodiments, R³ is —CF₃. In some embodiments, R³ is —CH₂CH₃. In some embodiments, R³ is —CH₂CH₂OH. In some embodiments, R³ is —CH₂CH₂F. In some embodiments, R³ is —CH₂CF₃. In some embodiments, R³ is —CH₂OCF₃. In some embodiments, R³ is —CH₂CH₂OCF₃. In some embodiments, R³ is —CH(CH₃)₂. In some embodiments, R³ is —CH₂COOH. In some embodiments, R³ is —CH₂CH₂COOH. In some embodiments, R³ is —CH₂CH(CH₃)₂. In some embodiments, R³ is —C(CH₃)₃. In some embodiments, R³ is —CH₂CH₂NH₂. In some embodiments, R³ is —CH₂CH₂NHCH₃. In some embodiments, R³ is —CH₂CH₂N(CH₃)₂. In some embodiments, R³ is —CH₂CH₂N(CH₃)₃⁺. In some embodiments, R³ is optionally substituted carbocyclyl or optionally substituted heterocyclyl. In some embodiments, R³ is selected from

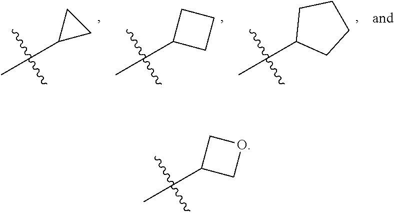

In some embodiments, R³ is

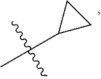 [SMILES: *C1CC1]

In some embodiments, R³ is

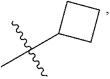 [SMILES: *C1CCC1]

In some embodiments, R³ is

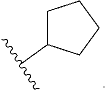 [SMILES: *C1CCCC1]

In some embodiments, R³ is

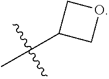 [SMILES: *C1COC1]

In some embodiments, n is 0, i.e., both R^{E} and R^{F} are absent. In some embodiments, n is 1, i.e., both R^{E} and R^{F} are present.

Optionally, at least one of R^{A} and R^{B} is hydrogen. In some embodiments, both R^{A} and R^{B} are hydrogen. In some embodiments, R^{A} is optionally substituted alkyl, and R^{B} is hydrogen. In some embodiments, R^{A} is optionally substituted C₁-C₄ alkyl, and R^{B} is hydrogen. In some embodiments, R^{A} is selected from —CH₃, —CF₃, —CH₂CH₃, —CH₂CH₂OH, —CH₂CH₂F, —CH₂CF₃, CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R^{B} is hydrogen. In some embodiments, R^{A} is methyl, and R^{B} is hydrogen.

Optionally, at least one of R^{A} and R^{B} is optionally substituted alkyl. In some embodiments, at least one of R^{A} and R^{B} is optionally substituted C₁-C₄ alkyl. In some embodiments, at least one of R^{A} and R^{B} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, at least one of R^{A} and R^{B} is methyl. In some embodiments, each of R^{A} and R^{B} is, independently, an optionally substituted alkyl. In some embodiments, each of R^{A} and R^{B} is, independently, an optionally substituted C₁-C₄ alkyl. In some embodiments, each of R^{A} and R^{B} is, independently, selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, each of R^{A} and R^{B} is methyl.

Optionally, at least one of R^{C} and R^{D} is hydrogen. In some embodiments, both R^{C} and R^{D} are hydrogen. In some embodiments, R^{C} is optionally substituted alkyl, and R^{D} is hydrogen. In some embodiments, R^{C} is optionally substituted C₁-C₄ alkyl, and R^{D} is hydrogen. In some embodiments, R^{C} is selected from —CH₃, —CF₃, —CH₂CH₃, —CH₂CH₂OH, —CH₂CH₂F, —CH₂CF₃, CH₂OCF₃, —CH₂CH₂OCF₃, —CH(CH₃)₂, —CH₂COOH, —CH₂CH₂COOH, —CH₂CH(CH₃)₂, C(CH₃)₃, —CH₂CH₂NH₂, —CH₂CH₂NHCH₃, —CH₂CH₂N(CH₃)₂, and —CH₂CH₂N(CH₃)₃⁺, and R^{D} is hydrogen. In some embodiments, R^{C} is methyl, and R^{D} is hydrogen.

Optionally, at least one of R^{C} and R^{D} is optionally substituted alkyl. In some embodiments, at least one of R^{C} and R^{D} is optionally substituted C₁-C₄ alkyl. In some embodiments, at least one of R^{C} and R^{D} is selected from —CH₃, —CF₃, —CH₂CH₃, CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, at least one of R^{C} and R^{D} is methyl. In some embodiments, each of R^{C} and R^{D} is, independently, an optionally substituted alkyl. In some embodiments, each of R^{C} and R^{D} is, independently, an optionally substituted C₁-C₄ alkyl. In some embodiments, each of R^{C} and R^{D} is, independently, selected from —CH₃, —CF₃, CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, C(CH₃)₃, —CH₂CH₂NH₂, —CH₂CH₂NHCH₃, —CH₂CH₂N(CH₃)₂, and —CH₂CH₂N(CH₃)₃⁺. In some embodiments, each of R^{C} and R^{D} is methyl.

In some embodiments, R^{A}, R^{B}, R^{C}, and R^{D} are hydrogen.

Optionally, at least one of R^{E} and R^{F} is hydrogen. In some embodiments, both R^{E} and R^{F} are hydrogen. In some embodiments, R^{E} is optionally substituted alkyl, and R^{F} is hydrogen. In some embodiments, R^{E} is optionally substituted C₁-C₄ alkyl, and R^{F} is hydrogen. In some embodiments, R^{E} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R^{F} is hydrogen. In some embodiments, R^{E} is methyl, and R^{F} is hydrogen.

Optionally, at least one of R^{E} and R^{F} is optionally substituted alkyl. In some embodiments, at least one of R^{E} and R^{F} is optionally substituted C₁-C₄ alkyl. In some embodiments, at least one of R^{E} and R^{F} is selected from —CH₃, —CF₃, —CH₂CH₃, CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, at least one of R^{E} and R^{F} is methyl. In some embodiments, each of R^{E} and R^{F} is, independently, an optionally substituted alkyl. In some embodiments, each of R^{E} and R^{F} is, independently, an optionally substituted C₁-C₄ alkyl. In some embodiments, each of R^{E} and R^{F} is, independently, selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, each of R^{E} and R^{F} is methyl.

In some embodiments, n is 0, X is NR¹R², and Y is NR³. In some embodiments, n is 0, X is NR¹R², Y is NR³, and R^{A} and R^{B} are hydrogen. In some embodiments, n is 0, X is NR¹R², Y is NR³, R^{A} is methyl, and R^{B} is hydrogen. In some embodiments, n is 0, X is NR¹R², Y is NR³, and R^{A} and R^{B} are methyl. In some embodiments, n is 0, X is NR¹R², Y is NR³, and R^{C} and R^{D} are hydrogen. In some embodiments, n is 0, X is NR¹R², Y is NR³, R^{C} is methyl, and R^{D} is hydrogen. In some embodiments, n is 0, X is NR¹R², Y is NR³, and R^{C} and R^{D} are methyl. In some embodiments, n is 0, X is NR¹R², Y is NR³, and R^{A}, R^{B}, R^{C}, and R^{D} are hydrogen. In some embodiments, both R¹ and R² are hydrogen. In some embodiments, R¹ is optionally substituted alkyl, and R² is hydrogen. In some embodiments, R¹ is optionally substituted C₁-C₄ alkyl, and R² is hydrogen. In some embodiments, R¹ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and CH₂CH₂N(CH₃)₃⁺, and R² is hydrogen. In some embodiments, R¹ is —CH₃, and R² is hydrogen. In some embodiments, R¹ is —CF₃, and R² is hydrogen. In some embodiments, R¹is —CH₂CH₃, and R² is hydrogen. In some embodiments, R¹ is —CH₂CH₂OH, and R² is hydrogen. In some embodiments, R¹ is —CH₂CH₂F, and R² is hydrogen. In some embodiments, R¹ is —CH₂CF₃, and R² is hydrogen. In some embodiments, R¹ is —CH₂OCF₃, and R² is hydrogen. In some embodiments, R¹ is —CH₂CH₂OCF₃, and R² is hydrogen. In some embodiments, R¹ is —CH(CH₃)₂, and R² is hydrogen. In some embodiments, R¹ is —CH₂COOH, and R² is hydrogen. In some embodiments, R¹ is —CH₂CH₂COOH, and R² is hydrogen. In some embodiments, R¹ is —CH₂CH(CH₃)₂, and R² is hydrogen. In some embodiments, R¹ is —C(CH₃)₃, and R² is hydrogen. In some embodiments, R¹ is —CH₂CH₂NH₂, and R² is hydrogen. In some embodiments, R¹ is —CH₂CH₂NHCH₃, and R² is hydrogen. In some embodiments, R¹ is —CH₂CH₂N(CH₃)₂, and R² is hydrogen. In some embodiments, R¹ is —CH₂CH₂N(CH₃)₃⁺, and R² is hydrogen. In some embodiments, R³ is hydrogen. In some embodiments, R³ is optionally substituted alkyl. In some embodiments, R³ is optionally substituted C₁-C₄ alkyl. In some embodiments, R³ is selected from CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, CH₂CH₂COOH, —CH₂CH(CH₃)₂, —C(CH₃)₃, CH₂CH₂NH₂, —CH₂CH₂NHCH₃, —CH₂CH₂N(CH₃)₂, and —CH₂CH₂N(CH₃)₃⁺. In some embodiments, R³ is —CH₃. In some embodiments, R³ is —CF₃. In some embodiments, R³ is —CH₂CH₃. In some embodiments, R³ is —CH₂CH₂OH. In some embodiments, R³ is —CH₂CH₂F. In some embodiments, R³ is —CH₂CF₃. In some embodiments, R³ is —CH₂OCF₃. In some embodiments, R³ is —CH₂CH₂OCF₃. In some embodiments, R³ is —CH(CH₃)₂. In some embodiments, R³ is —CH₂COOH. In some embodiments, R³ is —CH₂CH₂COOH. In some embodiments, R³ is —CH₂CH(CH₃)₂. In some embodiments, R³ is —C(CH₃)₃. In some embodiments, R³ is —CH₂CH₂NH₂. In some embodiments, R³ is —CH₂CH₂NHCH₃. In some embodiments, R³ is —CH₂CH₂N(CH₃)₂. In some embodiments, R³ is —CH₂CH₂N(CH₃)₃⁺. In some embodiments, R³ is optionally substituted carbocyclyl or optionally substituted heterocyclyl. In some embodiments, R³ is selected from

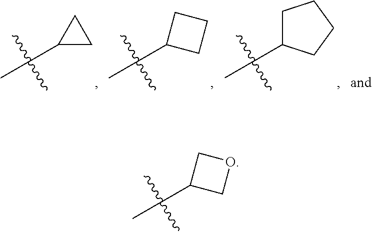

In some embodiments, R³ is

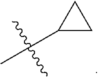 [SMILES: *C1CC1]

In some embodiments, R³ is

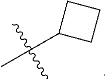 [SMILES: *C1CCC1]

In some embodiments, R³ is

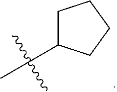 [SMILES: *C1CCCC1]

In some embodiments, R³ is

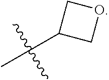 [SMILES: *C1COC1]

In some embodiments, n is 1, X is NR¹R², and Y is NR³. In some embodiments, n is 1, X is NR¹R², Y is NR³, and R^{A} and R^{B} are hydrogen. In some embodiments, n is 1, X is NR¹R², Y is NR³, R^{A} is methyl, and R^{B} is hydrogen. In some embodiments, n is 1, X is NR¹R², Y is NR³, and R^{A} and R^{B} are methyl. In some embodiments, n is 1, X is NR¹R², Y is NR³, and R^{C} and R^{D} are hydrogen. In some embodiments, n is 1, X is NR¹R², Y is NR³, R^{C} is methyl, and R^{D} is hydrogen. In some embodiments, n is 1, X is NR¹R², Y is NR³, and R^{C} and R^{D} are methyl. In some embodiments, n is 1, X is NR¹R², Y is NR³, and R^{E} and R^{F} are hydrogen. In some embodiments, n is 1, X is NR¹R², Y is NR³, R^{E} is methyl, and R^{F} is hydrogen. In some embodiments, n is 1, X is NR¹R², Y is NR³, and R^{E} and R^{F} are methyl. In some embodiments, n is 1, X is NR¹R², Y is NR³, and R^{A}, R^{B}, R^{C}, R^{D}, R^{E}, and R^{F} are hydrogen. In some embodiments, both R¹ and R² are hydrogen. In some embodiments, R¹ is optionally substituted alkyl, and R² is hydrogen. In some embodiments, R¹ is optionally substituted C₁-C₄ alkyl, and R² is hydrogen. In some embodiments, R¹ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, CH₂CH(CH₃)₂, —C(CH₃)₃, —CH₂CH₂NH₂, CH₂CH₂NHCH₃, —CH₂CH₂N(CH₃)₂, and —CH₂CH₂N(CH₃)₃⁺, and R² is hydrogen. In some embodiments, R¹ is —CH₃, and R² is hydrogen. In some embodiments, R¹ is —CF₃, and R² is hydrogen. In some embodiments, R¹ is —CH₂CH₃, and R² is hydrogen. In some embodiments, R¹ is —CH₂CH₂OH, and R² is hydrogen. In some embodiments, R¹ is —CH₂CH₂F, and R² is hydrogen. In some embodiments, R¹ is —CH₂CF₃, and R² is hydrogen. In some embodiments, R¹ is —CH₂OCF₃, and R² is hydrogen. In some embodiments, R¹ is —CH₂CH₂OCF₃, and R² is hydrogen. In some embodiments, R¹ is —CH(CH₃)₂, and R² is hydrogen. In some embodiments, R¹ is —CH₂COOH, and R² is hydrogen. In some embodiments, R¹ is —CH₂CH₂COOH, and R² is hydrogen. In some embodiments, R¹ is —CH₂CH(CH₃)₂, and R² is hydrogen. In some embodiments, R¹ is —C(CH₃)₃, and R² is hydrogen. In some embodiments, R¹ is —CH₂CH₂NH₂, and R² is hydrogen. In some embodiments, R¹ is —CH₂CH₂NHCH₃, and R² is hydrogen. In some embodiments, R¹ is —CH₂CH₂N(CH₃)₂, and R² is hydrogen. In some embodiments, R¹ is —CH₂CH₂N(CH₃)₃, and R² is hydrogen. In some embodiments, R³ is hydrogen. In some embodiments, R³ is optionally substituted alkyl. In some embodiments, R³ is optionally substituted C₁-C₄ alkyl. In some embodiments, R³ is selected from —CH₃, —CF₃, —CH₂CH₃, —CH₂CH₂OH, —CH₂CH₂F, —CH₂CF₃, CH₂OCF₃, —CH₂CH₂OCF₃, —CH(CH₃)₂, —CH₂COOH, —CH₂CH₂COOH, —CH₂CH(CH₃)₂, C(CH₃)₃, —CH₂CH₂NH₂, —CH₂CH₂NHCH₃, —CH₂CH₂N(CH₃)₂, and —CH₂CH₂N(CH₃)₃⁺. In some embodiments, R³ is —CH₃. In some embodiments, R³ is —CF₃. In some embodiments, R³ is —CH₂CH₃. In some embodiments, R³ is —CH₂CH₂OH. In some embodiments, R³ is —CH₂CH₂F. In some embodiments, R³ is —CH₂CF₃. In some embodiments, R³ is —CH₂OCF₃. In some embodiments, R³ is —CH₂CH₂OCF₃. In some embodiments, R³ is —CH(CH₃)₂. In some embodiments, R³ is —CH₂COOH. In some embodiments, R³ is —CH₂CH₂COOH. In some embodiments, R³ is —CH₂CH(CH₃)₂. In some embodiments, R³ is —C(CH₃)₃. In some embodiments, R³ is —CH₂CH₂NH₂. In some embodiments, R³ is —CH₂CH₂NHCH₃. In some embodiments, R³ is CH₂CH₂N(CH₃)₂. In some embodiments, R³ is —CH₂CH₂N(CH₃)₃⁺. In some embodiments, R³ is optionally substituted carbocyclyl or optionally substituted heterocyclyl. In some embodiments, R³ is selected from

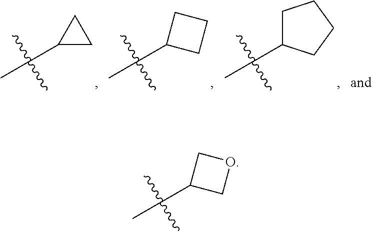

In some embodiments, R³ is

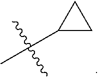 [SMILES: *C1CC1]

In some embodiments, R³ is

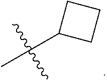 [SMILES: *C1CCC1]

In some embodiments, R³ is

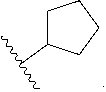 [SMILES: *C1CCCC1]

In some embodiments, R³ is

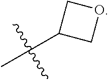 [SMILES: *C1COC1]

### Group II

In some embodiments, the compounds have the following features:
X is NR¹R²,
Y is O or NR³,
R¹ joins R^{C} or R^{E} to form a 4-7 membered, optionally substituted heterocycle,
R² is hydrogen,
R^{A}, R^{B}, R^{C}, R^{D}, R^{E}, and R^{F}, on each occurrence when not joined by R¹ to form the heterocycle, are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R³ is independently selected from hydrogen, optionally substituted alkyl, optionally substituted heteroalkyl, optionally substituted carbocyclyl, optionally substituted heterocyclyl, optionally substituted aryl, and optionally substituted heteroaryl.

In some embodiments, the 4-7 membered, optionally substituted heterocycle formed by R¹ joining R^{C} or R^{E} is unsubstituted.

In some embodiments, the 4-7 membered, optionally substituted heterocycle formed by R¹ joining R^{C} or R^{E} is substituted. Suitable substituents are in accordance with the general description of substitution in previous sections. In some embodiments, the 4-7 membered, optionally substituted heterocycle is substituted by one or more substituents independently selected from halogen, hydroxyl, —O (counting as two substituents), ═S (counting as two substituents), cyano, nitro, carboxyl, carbonate, alkyl, haloalkyl, carbocyclyl, halocarbocyclyl, heterocyclyl, haloheterocyclyl, aryl, haloaryl, acyl, sulfinyl, sulfonyl, sulfonate, sulfonamide, amide, optionally Si-substituted silyl, ester, thioester, carbonate ester, and carbamate. In some embodiments, the 4-7 membered, optionally substituted heterocycle is substituted by one or more substituents independently selected from halogen, hydroxyl, cyano, alkyl, polyfluoroalkyl, ═O, —S, amide, ester, sulfinyl, sulfonyl, sulfonate, and sulfonamide. In some embodiments, the 4-7 membered, optionally substituted heterocycle is substituted by one or more substituents independently selected from halogen, hydroxyl, ═O, ═S, amide, ester, alkyl, and polyfluoroalkyl. In some embodiments, the 4-7 membered, optionally substituted heterocycle is substituted by one or more substituents independently selected from fluorine, hydroxyl, ═O, ═S, —C(═O)NH₂, —C(═O)NHCH₃, C(═O)N(CH₃)₂, —C(═O)OCH(CH₃)₂, methyl, and trifluoromethyl. In some embodiments, the 4-7 membered, optionally substituted heterocycle is substituted by one or more fluorine atoms. In some embodiments, the 4-7 membered, optionally substituted heterocycle is substituted by a —C(═O)NH₂ group. In some embodiments, the 4-7 membered, optionally substituted heterocycle is substituted by a —C(═O)OCH(CH₃)₂ group. In some embodiments, the 4-7 membered, optionally substituted heterocycle is substituted by a ═O group.

In some embodiments, the 4-7 membered, optionally substituted heterocycle is a 5 or 6 membered, optionally substituted heterocycle. In some embodiments, the 4-7 membered, optionally substituted heterocycle is a 5-membered, optionally substituted heterocycle. In some embodiments, the 4-7 membered, optionally substituted heterocycle is a 6-membered, optionally substituted heterocycle. In some embodiments, the 4-7 membered, optionally substituted heterocycle is optionally substituted pyrrolidine or optionally substituted piperidine. In some embodiments, the 4-7 membered, optionally substituted heterocycle is optionally substituted pyrrolidine. In some embodiments, the 4-7 membered, optionally substituted heterocycle is pyrrolidine or piperidine. In some embodiments, the 4-7 membered, optionally substituted heterocycle is pyrrolidine.

In some embodiments, Y is O. In some embodiments, Y is NR³. In some embodiments, R³ is hydrogen. In some embodiments, R³ is optionally substituted alkyl. In some embodiments, R³ is optionally substituted C₁-C₄ alkyl. In some embodiments, R³ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, R³ is —CH₃. In some embodiments, R³ is —CF₃. In some embodiments, R³ is —CH₂CH₃. In some embodiments, R³ is —CH₂CH₂OH. In some embodiments, R³ is —CH₂CH₂F. In some embodiments, R³ is —CH₂CF₃. In some embodiments, R³ is —CH₂OCF₃. In some embodiments, R³ is —CH₂CH₂OCF₃. In some embodiments, R³ is —CH(CH₃)₂. In some embodiments, R³ is —CH₂COOH. In some embodiments, R³ is —CH₂CH₂COOH. In some embodiments, R³ is —CH₂CH(CH₃)₂. In some embodiments, R³ is —C(CH₃)₃. In some embodiments, R³ is —CH₂CH₂NH₂. In some embodiments, R³ is —CH₂CH₂NHCH₃. In some embodiments, R³ is —CH₂CH₂N(CH₃)₂. In some embodiments, R³ is —CH₂CH₂N(CH₃)₃⁺. In some embodiments, R³ is optionally substituted carbocyclyl or optionally substituted heterocyclyl. In some embodiments, R³ is selected from

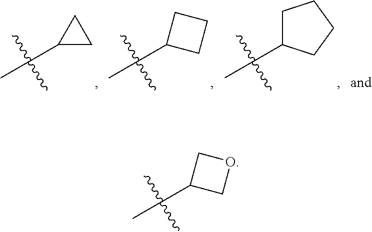

In some embodiments, R³ is

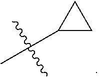 [SMILES: *C1CC1]

In some embodiments, R³ is

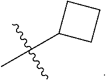 [SMILES: *C1CCC1]

In some embodiments, R³ is

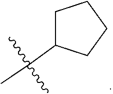 [SMILES: *C1CCCC1]

In some embodiments, R³ is

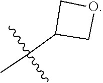 [SMILES: *C1COC1]

Optionally, at least one of R^{A} and R^{B} is hydrogen. In some embodiments, both R^{A} and R^{B} are hydrogen. In some embodiments, R^{A} is optionally substituted alkyl, and R^{B} is hydrogen. In some embodiments, R^{A} is optionally substituted C₁-C₄ alkyl, and R^{B} is hydrogen. In some embodiments, R^{A} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R^{B} is hydrogen. In some embodiments, R^{A} is methyl, and R^{B} is hydrogen.

Optionally, at least one of R^{A} and R^{B} is optionally substituted alkyl. In some embodiments, at least one of R^{A} and R^{B} is optionally substituted C₁-C₄ alkyl. In some embodiments, at least one of R^{A} and R^{B} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, at least one of R^{A} and R^{B} is methyl. In some embodiments, each of R^{A} and R^{B} is, independently, an optionally substituted alkyl. In some embodiments, each of R^{A} and R^{B} is, independently, an optionally substituted C₁-C₄ alkyl. In some embodiments, each of R^{A} and R^{B} is, independently, selected from —CH₃, CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, each of R^{A} and R^{B} is methyl.

In some embodiments, n is 0, i.e., both R^{E} and R^{F} are absent. When n is 0, R¹ can only join R^{C} to form the 4-7 membered, optionally substituted heterocycle.

In some embodiments, n is 1, i.e., both R^{E} and R^{F} are present. When n is 1, R¹ can join either R^{C} or R^{E} to form the 4-7 membered, optionally substituted heterocycle.

In some embodiments, R¹ joins R^{C} to form the 4-7 membered, optionally substituted heterocycle. When R¹ joins R^{C} to form the 4-7 membered, optionally substituted heterocycle, n can be 0 or 1.

In some embodiments, R¹ joins R^{E} to form the 4-7 membered, optionally substituted heterocycle. When R¹ joins R^{E} to form the 4-7 membered, optionally substituted heterocycle, n can only be 1.

Optionally, when R¹ joins R^{C} to form the 4-7 membered, optionally substituted heterocycle, R^{D} is hydrogen.

Optionally, when R¹ joins R^{C} to form the 4-7 membered, optionally substituted heterocycle, R^{D} is optionally substituted alkyl. In some embodiments, R^{D} is optionally substituted C₁-C₄ alkyl. In some embodiments, R^{D} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, CH₂CH(CH₃)₂, —C(CH₃)₃, —CH₂CH₂NH₂, —CH₂CH₂NHCH₃, —CH₂CH₂N(CH₃)₂, and CH₂CH₂N(CH₃)₃⁺. In some embodiments, R^{D} is methyl.

Optionally, when R¹ joins R^{C} to form the 4-7 membered, optionally substituted heterocycle and n is 0, both R^{E} and R^{F} are absent.

Optionally, when R¹ joins R^{C} to form the 4-7 membered, optionally substituted heterocycle and n is 1, at least one of R^{E} and R^{F} is hydrogen. In some embodiments, both R^{E} and R^{F} are hydrogen. In some embodiments, R^{E} is optionally substituted alkyl, and R^{F} is hydrogen. In some embodiments, R^{E} is optionally substituted C₁-C₄ alkyl, and R^{F} is hydrogen. In some embodiments, R^{E} is selected from —CH₃, —CF₃, —CH₂CH₃, —CH₂CH₂OH, —CH₂CH₂F, —CH₂CF₃, —CH₂OCF₃, CH₂CH₂OCF₃, —CH(CH₃)₂, —CH₂COOH, —CH₂CH₂COOH, CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R^{F} is hydrogen. In some embodiments, R^{E} is methyl, and R^{F} is hydrogen.

Optionally, when R¹ joins R^{C} to form the 4-7 membered, optionally substituted heterocycle and n is 1, at least one of R^{E} and R^{F} is optionally substituted alkyl. In some embodiments, at least one of R^{E} and R^{F} is optionally substituted C₁-C₄ alkyl. In some embodiments, at least one of R^{E} and R^{F} is selected from —CH₃, —CF₃, —CH₂CH₃, —CH₂CH₂OH, CH₂CH₂F, —CH₂CF₃, —CH₂OCF₃, CH₂CH₂OCF₃, —CH(CH₃)₂, CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, at least one of R^{E} and R^{F} is methyl. In some embodiments, each of R^{E} and R^{F} is, independently, an optionally substituted alkyl. In some embodiments, each of R^{E} and R^{F} is, independently, an optionally substituted C₁-C₄ alkyl. In some embodiments, each of R^{E} and R^{F} is, independently, selected from —CH₃, —CF₃, —CH₂CH₃, —CH₂CH₂OH, —CH₂CH₂F, —CH₂CF₃, CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, each of R^{E} and R^{F} is methyl.

Optionally, when R¹ joins R^{E} to form the 4-7 membered, optionally substituted heterocycle, R^{F} is hydrogen.

Optionally, when R¹ joins R^{E} to form the 4-7 membered, optionally substituted heterocycle, R^{F} is optionally substituted alkyl. In some embodiments, R^{F} is optionally substituted C₁-C₄ alkyl. In some embodiments, R^{F} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, R^{F} is methyl.

Optionally, when R¹ joins R^{E} to form the 4-7 membered, optionally substituted heterocycle, at least one of R^{C} and R^{D} is hydrogen. In some embodiments, both R^{C} and R^{D} are hydrogen. In some embodiments, R^{C} is optionally substituted alkyl, and R^{D} is hydrogen. In some embodiments, R^{C} is optionally substituted C₁-C₄ alkyl, and R^{D} is hydrogen. In some embodiments, R^{C} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R^{D} is hydrogen. In some embodiments, R^{C} is methyl, and R^{D} is hydrogen.

Optionally, when R¹ joins R^{E} to form the 4-7 membered, optionally substituted heterocycle, at least one of R^{C} and R^{D} is optionally substituted alkyl. In some embodiments, at least one of R^{C} and R^{D} is optionally substituted C₁-C₄ alkyl. In some embodiments, at least one of R^{C} and R^{D} is selected from —CH₃, —CF₃, —CH₂CH₃, —CH₂CH₂OH, —CH₂CH₂F, —CH₂CF₃, —CH₂OCF₃, CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, at least one of R^{C} and R^{D} is methyl. In some embodiments, each of R^{C} and R^{D} is, independently, an optionally substituted alkyl. In some embodiments, each of R^{C} and R^{D} is, independently, an optionally substituted C₁-C₄ alkyl. In some embodiments, each of R^{C} and R^{D} is, independently, selected from —CH₃, CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, each of R^{C} and R^{D} is methyl.

In some embodiments, when R¹ joins R^{C} to form the 4-7 membered, optionally substituted heterocycle, R^{A}, R^{B}, and R^{D} are hydrogen. In some embodiments, when R¹ joins R^{C} to form the 4-7 membered, optionally substituted heterocycle and n is 0, R^{A}, R^{B}, and R^{D} are hydrogen. In some embodiments, when R¹ joins R^{C} to form the 4-7 membered, optionally substituted heterocycle and n is 1, R^{A}, R^{B}, and R^{D} are hydrogen. In some embodiments, when R¹ joins R^{C} to form the 4-7 membered, optionally substituted heterocycle and n is 1, R^{A}, R^{B}, R^{D}, R^{E}, and R^{F} are hydrogen.

In some embodiments, when R¹ joins R^{E} to form the 4-7 membered, optionally substituted heterocycle, R^{A}, R^{B}, R^{C}, and R^{D} are hydrogen. In some embodiments, when R¹ joins R^{E} to form the 4-7 membered, optionally substituted heterocycle, R^{A}, R^{B}, R^{C}, R^{D}, and R^{F} are hydrogen.

In some embodiments, n is 0, and Y is NR³. In some embodiments, n is 0, Y is NR³, and R^{A} and R^{B} are hydrogen. In some embodiments, n is 0, Y is NR³, R^{A} is methyl, and R^{B} is hydrogen. In some embodiments, n is 0, Y is NR³, and R^{A} and R^{B} are methyl. In some embodiments, n is 0, Y is NR³, and R^{D} is hydrogen. In some embodiments, n is 0, Y is NR³, and R^{D} is methyl. In some embodiments, n is 0, Y is NR³, and R^{A}, R^{B}, and R^{D} are hydrogen. In some embodiments, R³ is hydrogen. In some embodiments, R³ is optionally substituted alkyl. In some embodiments, R³ is optionally substituted C₁-C₄ alkyl. In some embodiments, R³ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and CH₂CH₂N(CH₃)₃⁺. In some embodiments, R³ is —CH₃. In some embodiments, R³ is —CF₃. In some embodiments, R³ is —CH₂CH₃. In some embodiments, R³ is —CH₂CH₂OH. In some embodiments, R³ is —CH₂CH₂F. In some embodiments, R³ is —CH₂CF₃. In some embodiments, R³ is —CH₂OCF₃. In some embodiments, R³ is —CH₂CH₂OCF₃. In some embodiments, R³ is —CH(CH₃)₂. In some embodiments, R³ is —CH₂COOH. In some embodiments, R³ is —CH₂CH₂COOH. In some embodiments, R³ is —CH₂CH(CH₃)₂. In some embodiments, R³ is —C(CH₃)₃. In some embodiments, R³ is —CH₂CH₂NH₂. In some embodiments, R³ is —CH₂CH₂NHCH₃. In some embodiments, R³ is —CH₂CH₂N(CH₃)₂. In some embodiments, R³ is —CH₂CH₂N(CH₃)₃⁺. In some embodiments, R³ is optionally substituted carbocyclyl or optionally substituted heterocyclyl. In some embodiments, R³ is selected from

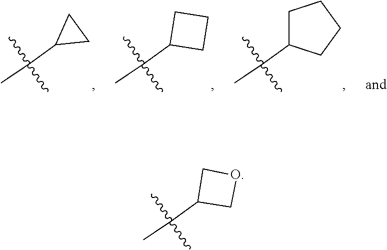

In some embodiments, R³ is

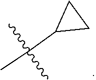 [SMILES: *C1CC1]

In some embodiments, R³ is

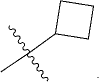 [SMILES: *C1CCC1]

In some embodiments, R³ is

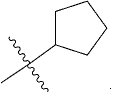 [SMILES: *C1CCCC1]

In some embodiments, R³ is

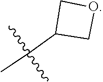 [SMILES: *C1COC1]

In some embodiments, n is 1, and Y is NR³.

In some embodiments, n is 1, Y is NR³, and R¹ joins R^{C} to form the 4-7 membered, optionally substituted heterocycle. In some embodiments, n is 1, Y is NR³, R¹ joins R^{C} to form the 4-7 membered, optionally substituted heterocycle, and R^{A} and R^{B} are hydrogen. In some embodiments, n is 1, Y is NR³, R¹ joins R^{C} to form the 4-7 membered, optionally substituted heterocycle, R^{A} is methyl, and R^{B} is hydrogen. In some embodiments, n is 1, Y is NR³, R¹ joins R^{C} to form the 4-7 membered, optionally substituted heterocycle, and R^{A} and R^{B} are methyl. In some embodiments, n is 1, Y is NR³, R¹ joins R^{C} to form the 4-7 membered, optionally substituted heterocycle, and R^{D} is hydrogen. In some embodiments, n is 1, Y is NR³, R¹ joins R^{C} to form the 4-7 membered, optionally substituted heterocycle, and R^{D} is methyl. In some embodiments, n is 1, Y is NR³, R¹ joins R^{C} to form the 4-7 membered, optionally substituted heterocycle, and R^{E} and R^{F} are hydrogen. In some embodiments, n is 1, Y is NR³, R¹ joins R^{C} to form the 4-7 membered, optionally substituted heterocycle, R^{E} is methyl, and R^{F} is hydrogen. In some embodiments, n is 1, Y is NR³, R¹ joins R^{C} to form the 4-7 membered, optionally substituted heterocycle, and R^{E} and R^{F} are methyl. In some embodiments, n is 1, Y is NR³, R¹ joins R^{C} to form the 4-7 membered, optionally substituted heterocycle, and R^{A}, R^{B}, R^{D}, R^{E}, and R^{F} are hydrogen. In some embodiments, R³ is hydrogen. In some embodiments, R³ is optionally substituted alkyl. In some embodiments, R³ is optionally substituted C₁-C₄ alkyl. In some embodiments, R³ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and CH₂CH₂N(CH₃)₃⁺. In some embodiments, R³ is —CH₃. In some embodiments, R³ is —CF₃. In some embodiments, R³ is —CH₂CH₃. In some embodiments, R³ is CH₂CH₂OH. In some embodiments, R³ is —CH₂CH₂F. In some embodiments, R³ is —CH₂CF₃. In some embodiments, R³ is —CH₂OCF₃. In some embodiments, R³ is —CH₂CH₂OCF₃. In some embodiments, R³ is —CH(CH₃)₂. In some embodiments, R³ is —CH₂COOH. In some embodiments, R³ is —CH₂CH₂COOH. In some embodiments, R³ is —CH₂CH(CH₃)₂. In some embodiments, R³ is —C(CH₃)₃. In some embodiments, R³ is —CH₂CH₂NH₂. In some embodiments, R³ is —CH₂CH₂NHCH₃. In some embodiments, R³ is —CH₂CH₂N(CH₃)₂. In some embodiments, R³ is —CH₂CH₂N(CH₃)₃⁺. In some embodiments, R³ is optionally substituted carbocyclyl or optionally substituted heterocyclyl. In some embodiments, R³ is selected from

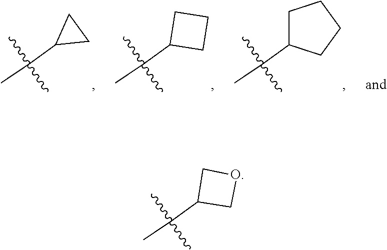

In some embodiments, R³ is

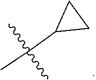 [SMILES: *C1CC1]

In some embodiments, R³ is

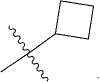 [SMILES: *C1CCC1]

In some embodiments, R³ is

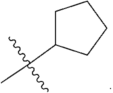 [SMILES: *C1CCCC1]

In some embodiments, R³ is

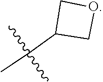 [SMILES: *C1COC1]

In some embodiments, n is 1, Y is NR³, and R¹ joins R^{E} to form the 4-7 membered, optionally substituted heterocycle. In some embodiments, n is 1, Y is NR³, R¹ joins R^{E} to form the 4-7 membered, optionally substituted heterocycle, and R^{A} and R^{B} are hydrogen. In some embodiments, n is 1, Y is NR³, R¹ joins R^{E} to form the 4-7 membered, optionally substituted heterocycle, R^{A} is methyl, and R^{B} is hydrogen. In some embodiments, n is 1, Y is NR³, R¹ joins R^{E} to form the 4-7 membered, optionally substituted heterocycle, and R^{A} and R^{B} are methyl. In some embodiments, n is 1, Y is NR³, R¹ joins R^{E} to form the 4-7 membered, optionally substituted heterocycle, and R^{C} and R^{D} are hydrogen. In some embodiments, n is 1, Y is NR³, R¹ joins R^{E} to form the 4-7 membered, optionally substituted heterocycle, R^{C} is methyl, and R^{D} is hydrogen. In some embodiments, n is 1, Y is NR³, R¹ joins R^{E} to form the 4-7 membered, optionally substituted heterocycle, and R^{C} and R^{D} are methyl. In some embodiments, n is 1, Y is NR³, R¹ joins R^{E} to form the 4-7 membered, optionally substituted heterocycle, and R^{F} is hydrogen. In some embodiments, n is 1, Y is NR³, R¹ joins R^{E} to form the 4-7 membered, optionally substituted heterocycle, and R^{F} is methyl. In some embodiments, n is 1, Y is NR³, R¹ joins R^{E} to form the 4-7 membered, optionally substituted heterocycle, and R^{A}, R^{B}, R^{C}, R^{D}, and R^{F} are hydrogen. In some embodiments, R³ is hydrogen. In some embodiments, R³ is optionally substituted alkyl. In some embodiments, R³ is optionally substituted C₁-C₄ alkyl. In some embodiments, R³ is selected from —CH₃, —CF₃, —CH₂CH₃, —CH₂CH₂OH, —CH₂CH₂F, —CH₂CF₃, —CH₂OCF₃, —CH₂CH₂OCF₃, CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, R³ is —CH₃. In some embodiments, R³ is —CF₃. In some embodiments, R³ is —CH₂CH₃. In some embodiments, R³ is —CH₂CH₂OH. In some embodiments, R³ is —CH₂CH₂F. In some embodiments, R³ is —CH₂CF₃. In some embodiments, R³ is —CH₂OCF₃. In some embodiments, R³ is —CH₂CH₂OCF₃. In some embodiments, R³ is —CH(CH₃)₂. In some embodiments, R³ is —CH₂COOH. In some embodiments, R³ is —CH₂CH₂COOH. In some embodiments, R³ is —CH₂CH(CH₃)₂. In some embodiments, R³ is —C(CH₃)₃. In some embodiments, R³ is —CH₂CH₂NH₂. In some embodiments, R³ is —CH₂CH₂NHCH₃. In some embodiments, R³ is —CH₂CH₂N(CH₃)₂. In some embodiments, R³ is —CH₂CH₂N(CH₃)₃⁺. In some embodiments, R³ is optionally substituted carbocyclyl or optionally substituted heterocyclyl. In some embodiments, R³ is selected from

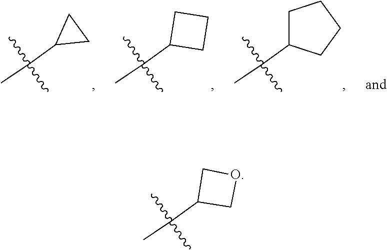

In some embodiments, R³ is

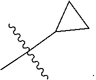 [SMILES: *C1CC1]

In some embodiments, R³ is

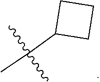 [SMILES: *C1CCC1]

In some embodiments, R³ is

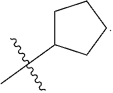 [SMILES: *C1CCCC1]

In some embodiments, R³ is

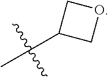 [SMILES: *C1COC1]

In some embodiments, Formula II is in the form of Formula IIC,

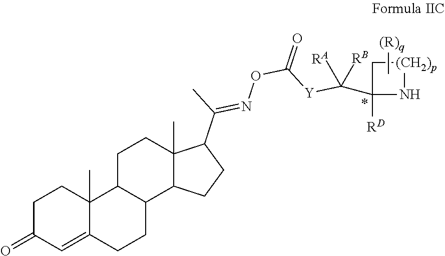

wherein Y, R^{A}, R^{B}, and R^{D} are the same as those described above in this section (i.e., Group II),
wherein p is an integer selected from 1-4,
wherein q is an integer selected from 0-6, and
wherein R is the same as those described above.

In some embodiments, the carbon atom labeled by the “*” sign is in a S configuration. In some embodiments, the carbon atom labeled by the “*” sign is in a R configuration.

In some embodiments, p is 1, 2, or 3. In some embodiments, p is 1. In some embodiments, p is 2. In some embodiments, p is 3.

In some embodiments, q is 0, 1, or 2. In some embodiments, q is 0. In some embodiments, q is 1. In some embodiments, q is 2.

In some embodiments, p is 1, 2, or 3, and q is 0, 1, or 2. In some embodiments, p is 2, and q is 0, 1, or 2. In some embodiments, p is 2, and q is 0. In some embodiments, p is 3, and q is 0, 1, or 2. In some embodiments, p is 3, and q is 0.

In some examples, the R groups are independently selected from halogen, nitro, cyano, hydroxyl, formyl, carboxyl, thiol, —O (counting as two R groups), ═S (counting two R groups), sulfamoyl, alkyl (such as methyl, ethyl, isopropyl, tert-butyl), haloalkyl (such as trifluoromethyl), alkenyl, haloalkenyl, alkynyl, haloalkynyl, carbocyclyl, halocarbocyclyl, heterocyclyl, haloheterocyclyl, aryl, haloaryl, heteroaryl, haloheteroaryl, arylalkyl (such as benzyl), alkylaryl, alkyloxy (such as methoxy, ethoxy), haloalkyloxy (such as trifluoromethoxy), aryloxy, alkylcarbonyl (such as acetyl), arylcarbonyl (such as benzoyl), alkylcarbonyloxy (such as acetoxy), arylcarbonyloxy (such as benzoyloxy), alkyloxycarbonyl (such as methoxycarbonyl, ethoxycarbonyl, isopropoxycarbonyl), aryloxycarbonyl, primary amino, alkylamino (such as methylamino, ethylamino, dimethylamino, diethylamino, N-methyl-N-ethylamino), alkylammonium (such as trimethylammonium), alkylcarbonylamino (such as acetylamino), arylcarbonylamino (such as benzoylamino), carbamoyl, N-alkylcarbamoyl (such as N-methylcarbamoyl, N-ethylcarbamoyl, N,N-dimethylcarbamoyl, N,N-diethylcarbamoyl, N-methyl-N-ethylcarbamoyl), alkylthio (such as methylthio, ethylthio), alkylsulfinyl (such as methylsulfinyl, ethylsulfinyl), alkylsulfonyl (such as mesyl, ethylsulfonyl), and N-alkylsulfamoyl (such as N-methylsulfamoyl, N-ethylsulfamoyl, N,N-dimethylsulfamoyl, N,N-diethylsulfamoyl, N-methyl-N-ethylsulfamoyl).

In some examples, the R groups are independently selected from halogen, nitro, cyano, hydroxyl, trifluoromethyl, methoxy, ethoxy, trifluoromethoxy, primary amino, formyl, carboxyl, carbamoyl, thiol, ═O, ═S, sulfamoyl, acetyl, acetoxy, methoxycarbonyl, ethoxycarbonyl, isopropoxycarbonyl, methylamino, ethylamino, dimethylamino, diethylamino, N-methyl-N-ethylamino, trimethylammonium, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, N-methylsulfamoyl, N-ethylsulfamoyl, N,N-dimethylsulfamoyl, N,N-diethylsulfamoyl, N-methyl-N-ethylsulfamoyl, benzyl, benzoyl, alkyl, haloalkyl, alkenyl, haloalkenyl, alkynyl, haloalkynyl, carbocyclyl, halocarbocyclyl, heterocyclyl, haloheterocyclyl, aryl, haloaryl, heteroaryl, and haloheteroaryl.

In some examples, the R groups are independently selected from halogen, ═O, ═S, alkyl, haloalkyl, carbocyclyl, halocarbocyclyl, aryl, haloaryl, heterocyclyl, and haloheterocyclyl.

In some examples, the R groups are independently selected from halogen, ═O, ═S, alkyl, haloalkyl, carbocyclyl, halocarbocyclyl, aryl, haloaryl, heterocyclyl, haloheterocyclyl, carbamoyl, N-alkylcarbamoyl, alkyloxycarbonyl, and aryloxycarbonyl.

In some examples, q is an integer selected from 1-6, and one of the R groups is carbamoyl or N-alkylcarbamoyl. For example, q is 1 and R is carbamoyl.

In some examples, q is an integer selected from 1-6, and one of the R groups is alkyloxycarbonyl or aryloxycarbonyl. For example, q is 1 and R is isopropoxycarbonyl.

In some examples, q is an integer selected from 1-6, and one of the R groups is fluorine. For example, q is 2 and both R groups are fluorine (they can be attached to the same atom or two different atoms).

In some examples, q is an integer selected from 2-6, and two of the R groups constitute ═O or ═S. For example, q is 2 and the two R groups constitute ═O.

In some embodiments, the

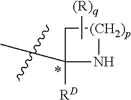

moiety in Formula IIC is selected from the following:

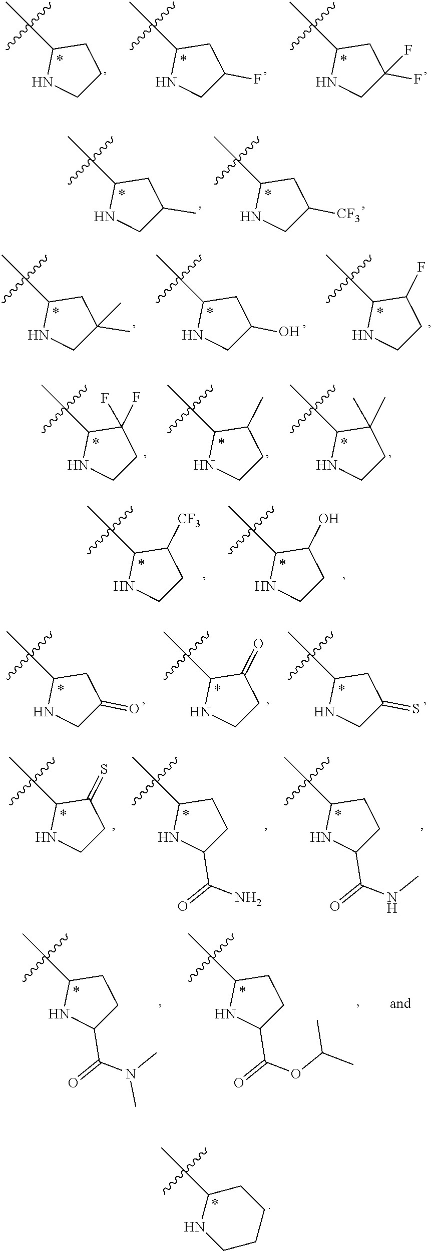

In some embodiments, the carbon atom labeled by the “*” sign is in a S configuration. In some embodiments, the carbon atom labeled by the “*” sign is in a R configuration.

In some embodiments, Y is NR³, and the

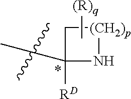

moiety in Formula IIC is selected from the following:

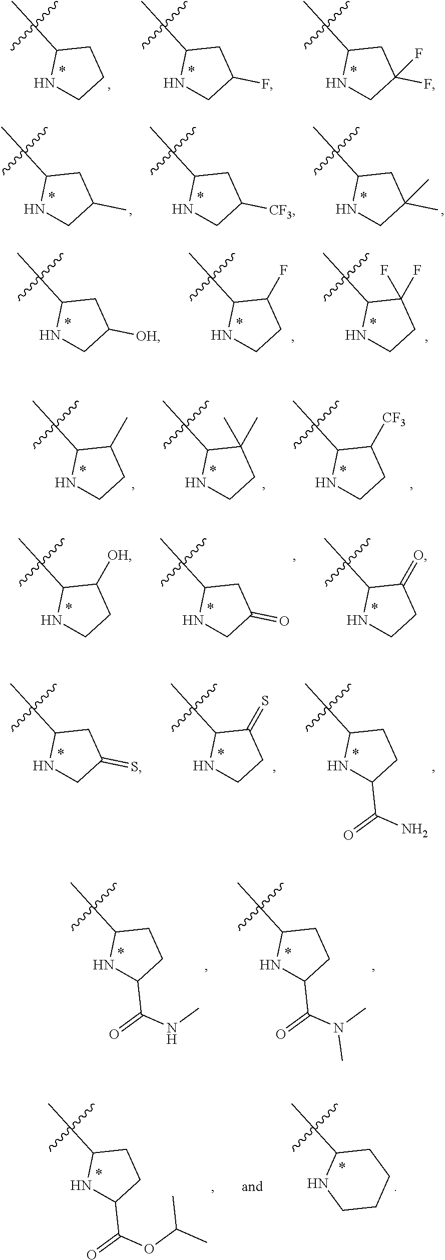

In some embodiments, the carbon atom labeled by the “*” sign is in a S configuration. In some embodiments, the carbon atom labeled by the “*” sign is in a R configuration. In some embodiments, R^{A} and R^{B} are hydrogen. In some embodiments, R^{A} is methyl, and R^{B} is hydrogen. In some embodiments, R^{A} and R^{B} are methyl. In some embodiments, R^{D} is hydrogen. In some embodiments, R^{D} is methyl. In some embodiments, R^{A}, R^{B}, and R^{D} are hydrogen. In some embodiments, R³ is hydrogen. In some embodiments, R³ is optionally substituted alkyl. In some embodiments, R³ is optionally substituted C₁-C₄ alkyl. In some embodiments, R³ is selected from —CH₃, —CF₃, CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, R³ is —CH₃. In some embodiments, R³ is —CF₃. In some embodiments, R³ is —CH₂CH₃. In some embodiments, R³ is —CH₂CH₂OH. In some embodiments, R³ is —CH₂CH₂F. In some embodiments, R³ is —CH₂CF₃. In some embodiments, R³ is —CH₂OCF₃. In some embodiments, R³ is —CH₂CH₂OCF₃. In some embodiments, R³ is —CH(CH₃)₂. In some embodiments, R³ is —CH₂COOH. In some embodiments, R³ is —CH₂CH₂COOH. In some embodiments, R³ is —CH₂CH(CH₃)₂. In some embodiments, R³ is —C(CH₃)₃. In some embodiments, R³ is —CH₂CH₂NH₂. In some embodiments, R³ is —CH₂CH₂NHCH₃. In some embodiments, R³ is —CH₂CH₂N(CH₃)₂. In some embodiments, R³ is CH₂CH₂N(CH₃)₃⁺. In some embodiments, R³ is optionally substituted carbocyclyl or optionally substituted heterocyclyl. In some embodiments, R³ is selected from

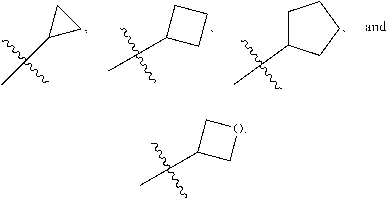

In some embodiments, R³ is

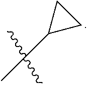 [SMILES: *C1CC1]

In some embodiments, R³ is

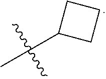 [SMILES: *C1CCC1]

In some embodiments, R³ is

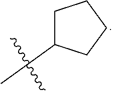 [SMILES: *C1CCCC1]

In some embodiments, R³ is

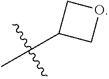 [SMILES: *C1COC1]

In some embodiments, Formula II is in the form of Formula IID,

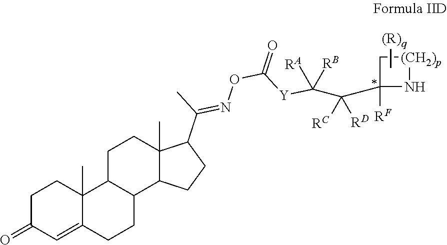

wherein Y, R^{A}, R^{B}, R^{C}, R^{D}, and R^{F} are the same as those described above in this section (i.e., Group II),
wherein p is an integer selected from 1-4,
wherein q is an integer selected from 0-6, and
wherein R is the same as those described above.

In some embodiments, the carbon atom labeled by the “*” sign is in a S configuration. In some embodiments, the carbon atom labeled by the “*” sign is in a R configuration.

In some embodiments, p is 1, 2, or 3. In some embodiments, p is 1. In some embodiments, p is 2. In some embodiments, p is 3.

In some embodiments, q is 0, 1, or 2. In some embodiments, q is 0. In some embodiments, q is 1. In some embodiments, q is 2.

In some embodiments, p is 1, 2, or 3, and q is 0, 1, or 2. In some embodiments, p is 2, and q is 0, 1, or 2. In some embodiments, p is 2, and q is 0. In some embodiments, p is 3, and q is 0, 1, or 2. In some embodiments, p is 3, and q is 0.

In some examples, the R groups are independently selected from halogen, nitro, cyano, hydroxyl, formyl, carboxyl, thiol, ═O (counting as two R groups), ═S (counting two R groups), sulfamoyl, alkyl (such as methyl, ethyl, isopropyl, tert-butyl), haloalkyl (such as trifluoromethyl), alkenyl, haloalkenyl, alkynyl, haloalkynyl, carbocyclyl, halocarbocyclyl, heterocyclyl, haloheterocyclyl, aryl, haloaryl, heteroaryl, haloheteroaryl, arylalkyl (such as benzyl), alkylaryl, alkyloxy (such as methoxy, ethoxy), haloalkyloxy (such as trifluoromethoxy), aryloxy, alkylcarbonyl (such as acetyl), arylcarbonyl (such as benzoyl), alkylcarbonyloxy (such as acetoxy), arylcarbonyloxy (such as benzoyloxy), alkyloxycarbonyl (such as methoxycarbonyl, ethoxycarbonyl, isopropoxycarbonyl), aryloxycarbonyl, primary amino, alkylamino (such as diethylamino, N-methyl-N-ethylamino), methylamino, ethylamino, dimethylamino, alkylammonium (such as trimethylammonium), alkylcarbonylamino (such as acetylamino), arylcarbonylamino (such as benzoylamino), carbamoyl, N-alkylcarbamoyl (such as N-methylcarbamoyl, N-ethylcarbamoyl, N,N-dimethylcarbamoyl, N,N-diethylcarbamoyl, N-methyl-N-ethylcarbamoyl), alkylthio (such as methylthio, ethylthio), alkylsulfinyl (such as methylsulfinyl, ethylsulfinyl), alkylsulfonyl (such as mesyl, ethylsulfonyl), and N-alkylsulfamoyl (such as N-methylsulfamoyl, N-ethylsulfamoyl, N,N-dimethylsulfamoyl, N,N-diethylsulfamoyl, N-methyl-N-ethylsulfamoyl).

In some examples, the R groups are independently selected from halogen, nitro, cyano, hydroxyl, trifluoromethyl, methoxy, ethoxy, trifluoromethoxy, primary amino, formyl, carboxyl, carbamoyl, thiol, ═O, ═S, sulfamoyl, acetyl, acetoxy, methoxycarbonyl, ethoxycarbonyl, isopropoxycarbonyl, methylamino, ethylamino, dimethylamino, diethylamino, N-methyl-N-ethylamino, trimethylammonium, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, N-methylsulfamoyl, N-ethylsulfamoyl, N,N-dimethylsulfamoyl, N,N-diethylsulfamoyl, N-methyl-N-ethylsulfamoyl, benzyl, benzoyl, alkyl, haloalkyl, alkenyl, haloalkenyl, alkynyl, haloalkynyl, carbocyclyl, halocarbocyclyl, heterocyclyl, haloheterocyclyl, aryl, haloaryl, heteroaryl, and haloheteroaryl.

In some examples, the R groups are independently selected from halogen, ═O, ═S, alkyl, haloalkyl, carbocyclyl, halocarbocyclyl, aryl, haloaryl, heterocyclyl, and haloheterocyclyl.

In some examples, the R groups are independently selected from halogen, ═O, ═S, alkyl, haloalkyl, carbocyclyl, halocarbocyclyl, aryl, haloaryl, heterocyclyl, haloheterocyclyl, carbamoyl, N-alkylcarbamoyl, alkyloxycarbonyl, and aryloxycarbonyl.

In some examples, q is an integer selected from 1-6, and one of the R groups is carbamoyl or N-alkylcarbamoyl. For example, q is 1 and R is carbamoyl.

In some examples, q is an integer selected from 1-6, and one of the R groups is alkyloxycarbonyl or aryloxycarbonyl. For example, q is 1 and R is isopropoxycarbonyl.

In some examples, q is an integer selected from 1-6, and one of the R groups is fluorine. For example, q is 2 and both R groups are fluorine (they can be attached to the same atom or two different atoms).

In some examples, q is an integer selected from 2-6, and two of the R groups constitute ═O or ═S. For example, q is 2 and the two R groups constitute ═O.

In some embodiments, the

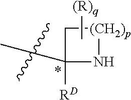

moiety in Formula IID is selected from the following:

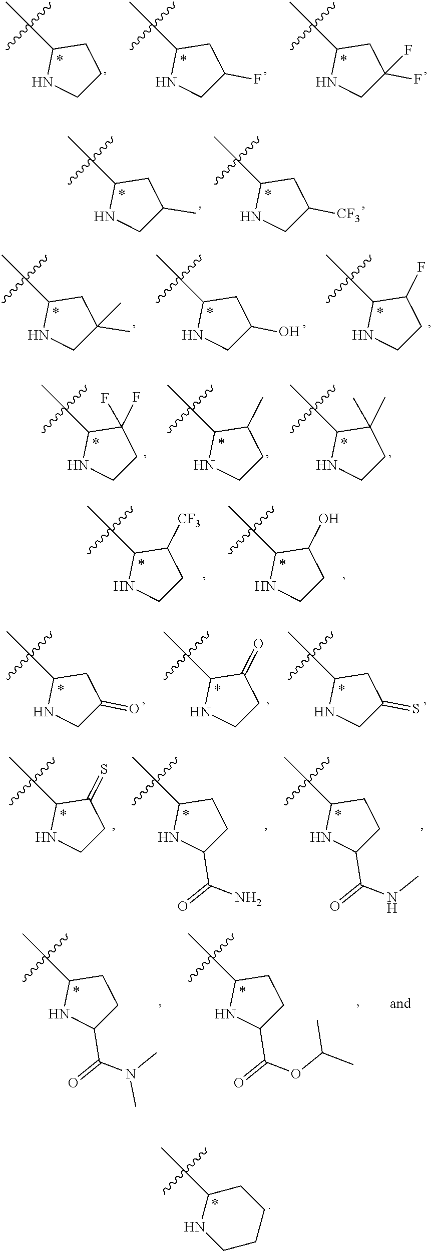

In some embodiments, the carbon atom labeled by the “*” sign is in a S configuration. In some embodiments, the carbon atom labeled by the “*” sign is in a R configuration.

In some embodiments, Y is NR³, and the

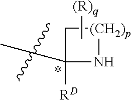

moiety in Formula IID is selected from the following:

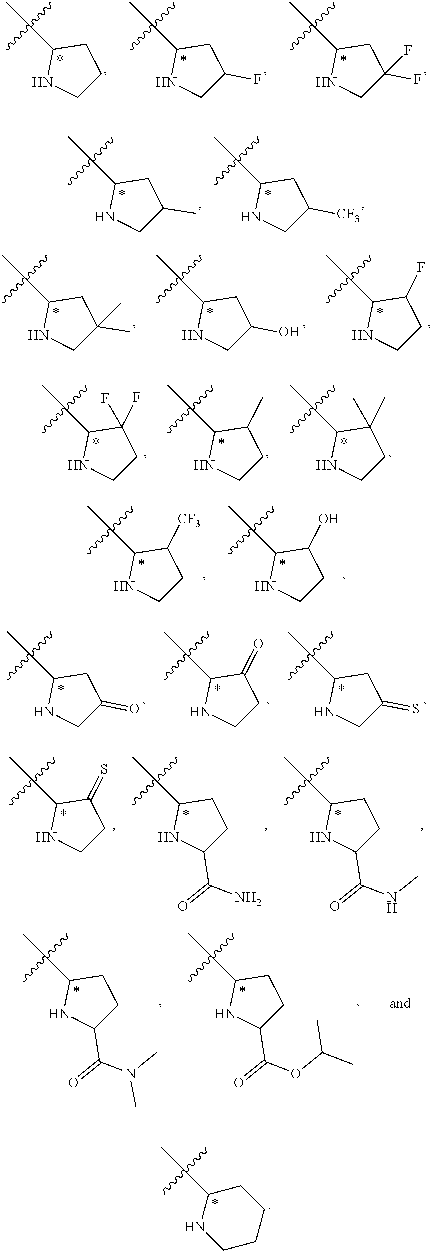

In some embodiments, the carbon atom labeled by the “*” sign is in a S configuration. In some embodiments, the carbon atom labeled by the “*” sign is in a R configuration. In some embodiments, R^{A} and R^{B} are hydrogen. In some embodiments, R^{A} is methyl, and R^{B} is hydrogen. In some embodiments, R^{A} and R^{B} are methyl. In some embodiments, R^{C} and R^{D} are hydrogen. In some embodiments, R^{C} is methyl, and R^{D} is hydrogen. In some embodiments, and R^{C} and R^{D} are methyl. In some embodiments, R^{F} is hydrogen. In some embodiments, R^{F} is methyl. In some embodiments, R^{A}, R^{B}, R^{C}, R^{D}, and R^{F} are hydrogen. In some embodiments, R³ is hydrogen. In some embodiments, R³ is optionally substituted alkyl. In some embodiments, R³ is optionally substituted C₁-C₄ alkyl. In some embodiments, R³ is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, R³ is —CH₃. In some embodiments, R³ is —CF₃. In some embodiments, R³ is —CH₂CH₃. In some embodiments, R³ is CH₂CH₂OH. In some embodiments, R³ is —CH₂CH₂F. In some embodiments, R³ is —CH₂CF₃. In some embodiments, R³ is —CH₂OCF₃. In some embodiments, R³ is —CH₂CH₂OCF₃. In some embodiments, R³ is —CH(CH₃)₂. In some embodiments, R³ is —CH₂COOH. In some embodiments, R³ is —CH₂CH₂COOH. In some embodiments, R³ is —CH₂CH(CH₃)₂. In some embodiments, R³ is —C(CH₃)₃. In some embodiments, R³ is —CH₂CH₂NH₂. In some embodiments, R³ is —CH₂CH₂NHCH₃. In some embodiments, R³ is —CH₂CH₂N(CH₃)₂. In some embodiments, R³ is —CH₂CH₂N(CH₃)₃⁺. In some embodiments, R³ is optionally substituted carbocyclyl or optionally substituted heterocyclyl. In some embodiments, R³ is selected from

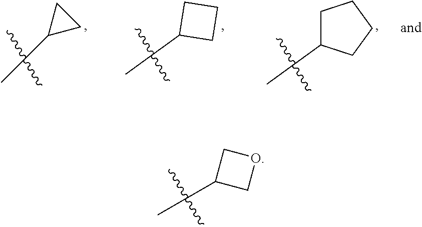

In some embodiments, R³ is

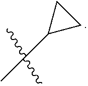 [SMILES: *C1CC1]

In some embodiments, R³ is

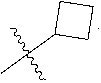 [SMILES: *C1CCC1]

In some embodiments, R³ is

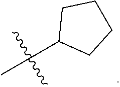 [SMILES: *C1CCCC1]

In some embodiments, R³ is

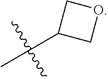 [SMILES: *C1COC1]

### Group III

In some embodiments, the compounds have the following features:
X is OH or NR¹R²,
Y is NR³,
R³ joins R^{A} to form a 4-7 membered, optionally substituted heterocycle,
R^{B}, R^{C}, R^{D}, R^{E}, and R^{F} are independently selected from hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R¹ and R² are independently selected from hydrogen, optionally substituted alkyl, optionally substituted heteroalkyl, optionally substituted carbocyclyl, optionally substituted heterocyclyl, optionally substituted aryl, and optionally substituted heteroaryl, with the proviso that at least one of R¹ and R² is hydrogen.

In some embodiments, the 4-7 membered, optionally substituted heterocycle formed by R³ joining R^{A} is unsubstituted.

In some embodiments, the 4-7 membered, optionally substituted heterocycle formed by R³ joining R^{A} is substituted. Suitable substituents are in accordance with the general description of substitution in previous sections. In some embodiments, the 4-7 membered, optionally substituted heterocycle is substituted by one or more substituents independently selected from halogen, hydroxyl, ═O (counting as two substituents), ═S (counting as two substituents), cyano, nitro, carboxyl, carbonate, alkyl, haloalkyl, carbocyclyl, halocarbocyclyl, heterocyclyl, haloheterocyclyl, aryl, haloaryl, acyl, sulfinyl, sulfonyl, sulfonate, sulfonamide, amide, optionally Si-substituted silyl, ester, thioester, carbonate ester, and carbamate. In some embodiments, the 4-7 membered, optionally substituted heterocycle is substituted by one or more substituents independently selected from halogen, hydroxyl, cyano, alkyl, polyfluoroalkyl, ═O, ═S, amide, ester, sulfinyl, sulfonyl, sulfonate, and sulfonamide. In some embodiments, the 4-7 membered, optionally substituted heterocycle is substituted by one or more substituents independently selected from halogen, hydroxyl, ═O, ═S, amide, ester, alkyl, and polyfluoroalkyl. In some embodiments, the 4-7 membered, optionally substituted heterocycle is substituted by one or more substituents independently selected from fluorine, hydroxyl, ═O, ═S, —C(═O)NH₂, —C(═O)NHCH₃, C(═O)N(CH₃)₂, —C(═O)OCH(CH₃)₂, methyl, and trifluoromethyl. In some embodiments, the 4-7 membered, optionally substituted heterocycle is substituted by one or more fluorine atoms. In some embodiments, the 4-7 membered, optionally substituted heterocycle is substituted by a —C(═O)NH₂ group. In some embodiments, the 4-7 membered, optionally substituted heterocycle is substituted by a —C(═O)OCH(CH₃)₂ group. In some embodiments, the 4-7 membered, optionally substituted heterocycle is substituted by a ═O group.

In some embodiments, the 4-7 membered, optionally substituted heterocycle is a 5 or 6 membered, optionally substituted heterocycle. In some embodiments, the 4-7 membered, optionally substituted heterocycle is a 5-membered, optionally substituted heterocycle. In some embodiments, the 4-7 membered, optionally substituted heterocycle is a 6-membered, optionally substituted heterocycle. In some embodiments, the 4-7 membered, optionally substituted heterocycle is optionally substituted pyrrolidine or optionally substituted piperidine. In some embodiments, the 4-7 membered, optionally substituted heterocycle is optionally substituted pyrrolidine. In some embodiments, the 4-7 membered, optionally substituted heterocycle is pyrrolidine or piperidine. In some embodiments, the 4-7 membered, optionally substituted heterocycle is pyrrolidine.

In some embodiments, X is OH. In some embodiments, X is NR¹R². In some embodiments, both R¹ and R² are hydrogen. In some embodiments, R¹ is optionally substituted alkyl, and R² is hydrogen. In some embodiments, R¹ is optionally substituted C₁-C₄ alkyl, and R² is hydrogen. In some embodiments, R¹ is selected from —CH₃, —CF₃, —CH₂CH₃, —CH₂CH₂OH, CH₂CH₂F, —CH₂CF₃, —CH₂OCF₃, —CH₂CH₂OCF₃, —CH(CH₃)₂, —CH₂COOH, —CH₂CH₂COOH, CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R² is hydrogen. In some embodiments, R¹ is —CH₃, and R² is hydrogen. In some embodiments, R¹ is —CF₃, and R² is hydrogen. In some embodiments, R¹ is —CH₂CH₃, and R² is hydrogen. In some embodiments, R¹ is —CH₂CH₂OH, and R² is hydrogen. In some embodiments, R¹ is —CH₂CH₂F, and R² is hydrogen. In some embodiments, R¹ is —CH₂CF₃, and R² is hydrogen. In some embodiments, R¹ is —CH₂OCF₃, and R² is hydrogen. In some embodiments, R¹ is —CH₂CH₂OCF₃, and R² is hydrogen. In some embodiments, R¹ is —CH(CH₃)₂, and R² is hydrogen. In some embodiments, R¹ is —CH₂COOH, and R² is hydrogen. In some embodiments, R¹ is —CH₂CH₂COOH, and R² is hydrogen. In some embodiments, R¹ is —CH₂CH(CH₃)₂, and R² is hydrogen. In some embodiments, R¹ is —C(CH₃)₃, and R² is hydrogen. In some embodiments, R¹ is —CH₂CH₂NH₂, and R² is hydrogen. In some embodiments, R¹ is CH₂CH₂NHCH₃, and R² is hydrogen. In some embodiments, R¹ is —CH₂CH₂N(CH₃)₂, and R² is hydrogen. In some embodiments, R¹ is —CH₂CH₂N(CH₃)₃⁺, and R² is hydrogen.

In some embodiments, n is 0, i.e., both R^{E} and R^{F} are absent. In some embodiments, n is 1, i.e., both R^{E} and R^{F} are present.

Optionally, R^{B} is hydrogen.

Optionally, R^{B} is optionally substituted alkyl. In some embodiments, R^{B} is optionally substituted C₁-C₄ alkyl. In some embodiments, R^{B} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, CH₂CH(CH₃)₂, —C(CH₃)₃, —CH₂CH₂NH₂, —CH₂CH₂NHCH₃, CH₂CH₂N(CH₃)₂, and —CH₂CH₂N(CH₃)₃⁺. In some embodiments, R^{B} is methyl.

Optionally, at least one of R^{C} and R^{D} is hydrogen. In some embodiments, both R^{C} and R^{D} are hydrogen. In some embodiments, R^{C} is optionally substituted alkyl, and R^{D} is hydrogen. In some embodiments, R^{C} is optionally substituted C₁-C₄ alkyl, and R^{D} is hydrogen. In some embodiments, R^{C} is selected from —CH₃, —CF₃, —CH₂CH₃, CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, CH₂CH₂N(CH₃)₂, and —CH₂CH₂N(CH₃)₃⁺, and R^{D} is hydrogen. In some embodiments, R^{C} is methyl, and R^{D} is hydrogen.

Optionally, at least one of R^{C} and R^{D} is optionally substituted alkyl. In some embodiments, at least one of R^{C} and R^{D} is optionally substituted C₁-C₄ alkyl. In some embodiments, at least one of R^{C} and R^{D} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, at least one of R^{C} and R^{D} is methyl. In some embodiments, each of R^{C} and R^{D} is, independently, an optionally substituted alkyl. In some embodiments, each of R^{C} and R^{D} is, independently, an optionally substituted C₁-C₄ alkyl. In some embodiments, each of R^{C} and R^{D} is, independently, selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, each of R^{C} and R^{D} is methyl.

In some embodiments, R^{B}, R^{C}, and R^{D} are hydrogen.

Optionally, at least one of R^{E} and R^{F} is hydrogen. In some embodiments, both R^{E} and R^{F} are hydrogen. In some embodiments, R^{E} is optionally substituted alkyl, and R^{F} is hydrogen. In some embodiments, R^{E} is optionally substituted C₁-C₄ alkyl, and R^{F} is hydrogen. In some embodiments, R^{E} is selected from —CH₃, —CF₃, —CH₂CH₃, —CH₂CH₂OH, —CH₂CH₂F, —CH₂CF₃, CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R^{F} is hydrogen. In some embodiments, R^{E} is methyl, and R^{F} is hydrogen.

Optionally, at least one of R^{E} and R^{F} is optionally substituted alkyl. In some embodiments, at least one of R^{E} and R^{F} is optionally substituted C₁-C₄ alkyl. In some embodiments, at least one of R^{E} and R^{F} is selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, at least one of R^{E} and R^{F} is methyl. In some embodiments, each of R^{E} and R^{F} is, independently, an optionally substituted alkyl. In some embodiments, each of R^{E} and R^{F} is, independently, an optionally substituted C₁-C₄ alkyl. In some embodiments, each of R^{E} and R^{F} is, independently, selected from —CH₃, —CF₃, —CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, each of R^{E} and R^{F} is methyl.

In some embodiments, n is 0, X is NR¹R², and Y is NR³. In some embodiments, n is 0, X is NR R², Y is NR³, and R^{B} is hydrogen. In some embodiments, n is 0, X is NR¹R², Y is NR³, and R^{B} is methyl. In some embodiments, n is 0, X is NR¹R², Y is NR³, and R^{C} and R^{D} are hydrogen. In some embodiments, n is 0, X is NR¹R², Y is NR³, R^{C} is methyl, and R^{D} is hydrogen. In some embodiments, n is 0, X is NR¹R², Y is NR³, and R^{C} and R^{D} are methyl. In some embodiments, n is 0, X is NR R², Y is NR³, and R^{B}, R^{C}, and R^{D} are hydrogen. In some embodiments, both R¹ and R² are hydrogen. In some embodiments, R¹ is optionally substituted alkyl, and R² is hydrogen. In some embodiments, R¹ is optionally substituted C₁-C₄ alkyl, and R² is hydrogen. In some embodiments, R¹ is selected from —CH₃, —CF₃, —CH₂CH₃, —CH₂CH₂OH, —CH₂CH₂F, —CH₂CF₃, CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺, and R² is hydrogen. In some embodiments, R¹ is —CH₃, and R² is hydrogen. In some embodiments, R¹ is —CF₃, and R² is hydrogen. In some embodiments, R¹ is —CH₂CH₃, and R² is hydrogen. In some embodiments, R¹ is —CH₂CH₂OH, and R² is hydrogen. In some embodiments, R¹ is —CH₂CH₂F, and R² is hydrogen. In some embodiments, R¹ is —CH₂CF₃, and R² is hydrogen. In some embodiments, R¹ is —CH₂OCF₃, and R² is hydrogen. In some embodiments, R¹ is —CH₂CH₂OCF₃, and R² is hydrogen. In some embodiments, R¹ is —CH(CH₃)₂, and R² is hydrogen. In some embodiments, R¹ is —CH₂COOH, and R² is hydrogen. In some embodiments, R¹ is CH₂CH₂COOH, and R² is hydrogen. In some embodiments, R¹ is —CH₂CH(CH₃)₂, and R² is hydrogen. In some embodiments, R¹ is —C(CH₃)₃, and R² is hydrogen. In some embodiments, R¹ is —CH₂CH₂NH₂, and R² is hydrogen. In some embodiments, R¹ is —CH₂CH₂NHCH₃, and R² is hydrogen. In some embodiments, R¹ is —CH₂CH₂N(CH₃)₂, and R² is hydrogen. In some embodiments, R¹ is —CH₂CH₂N(CH₃)₃⁺, and R² is hydrogen.

In some embodiments, n is 1, X is NR¹R², and Y is NR³. In some embodiments, n is 1, X is NR¹R², Y is NR³, and R^{B} is hydrogen. In some embodiments, n is 1, X is NR¹R², Y is NR³, and R^{B} is methyl. In some embodiments, n is 1, X is NR R², Y is NR³, and R^{C} and R^{D} are hydrogen. In some embodiments, n is 1, X is NR¹R², Y is NR³, R^{C} is methyl, and R^{D} is hydrogen. In some embodiments, n is 1, X is NR R², Y is NR³, and R^{C} and R^{D} are methyl. In some embodiments, n is 1, X is NR 1R², Y is NR³, and R^{E} and R^{F} are hydrogen. In some embodiments, n is 1, X is NR¹R², Y is NR³, R^{E} is methyl, and R^{F} is hydrogen. In some embodiments, n is 1, X is NR¹R², Y is NR³, and R^{E} and R^{F} are methyl. In some embodiments, both R¹ and R² are hydrogen. In some embodiments, R¹ is optionally substituted alkyl, and R² is hydrogen. In some embodiments, R¹ is optionally substituted C₁-C₄ alkyl, and R² is hydrogen. In some embodiments, R¹ is selected from —CH₃, —CF₃, —CH₂CH₃, —CH₂CH₂OH, CH₂CH₂F, CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, CH₂CH₂NHCH₃, —CH₂CH₂N(CH₃)₂, and —CH₂CH₂N(CH₃)₃⁺, and R² is hydrogen. In some embodiments, R¹ is —CH₃, and R² is hydrogen. In some embodiments, R¹ is —CF₃, and R² is hydrogen. In some embodiments, R¹ is —CH₂CH₃, and R² is hydrogen. In some embodiments, R¹ is —CH₂CH₂OH, and R² is hydrogen. In some embodiments, R¹ is —CH₂CH₂F, and R² is hydrogen. In some embodiments, R¹ is —CH₂CF₃, and R² is hydrogen. In some embodiments, R¹ is —CH₂OCF₃, and R² is hydrogen. In some embodiments, R¹ is —CH₂CH₂OCF₃, and R² is hydrogen. In some embodiments, R¹ is —CH(CH₃)₂, and R² is hydrogen. In some embodiments, R¹ is —CH₂COOH, and R² is hydrogen. In some embodiments, R¹ is —CH₂CH₂COOH, and R² is hydrogen. In some embodiments, R¹ is —CH₂CH(CH₃)₂, and R² is hydrogen. In some embodiments, R¹ is —C(CH₃)₃, and R² is hydrogen. In some embodiments, R¹ is —CH₂CH₂NH₂, and R² is hydrogen. In some embodiments, R¹ is —CH₂CH₂NHCH₃, and R² is hydrogen. In some embodiments, R¹ is CH₂CH₂N(CH₃)₂, and R² is hydrogen. In some embodiments, R¹ is —CH₂CH₂N(CH₃)₃⁺, and R² is hydrogen.

### B. Exemplary Structures

In some embodiments, the compounds are progesterone derivatives having a structure of Formula II-1 or a pharmaceutically acceptable salt, hydrate, or hydrated salt thereof,

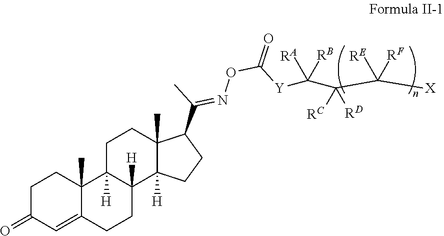

wherein X, Y, R^{A}, R^{B}, R^{C}, R^{D}, R^{E}, R^{F}, and n are the same as described in Formula II.

In some embodiments, the compounds are in a non-salt form as shown in Formula II-1. In some embodiments, the compounds are in a salt form. In some embodiments, the compounds are in an HCl salt form.

In some embodiments, n is 0, and Formula II-1 is in the form of Formula II-1A.

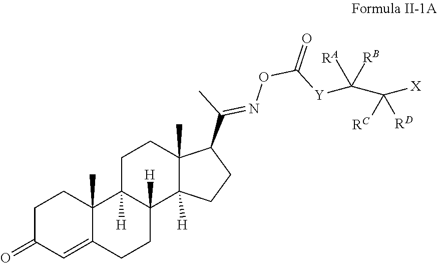

In some embodiments, n is 1, and Formula II-1 is in the form of Formula II-1B.

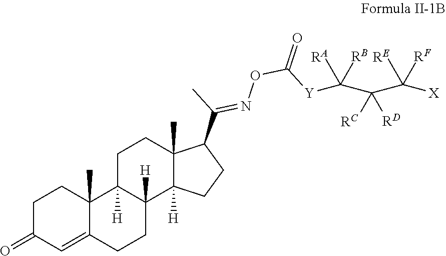

In some embodiments, Formula II-1 is in the form of Formula II-1C,

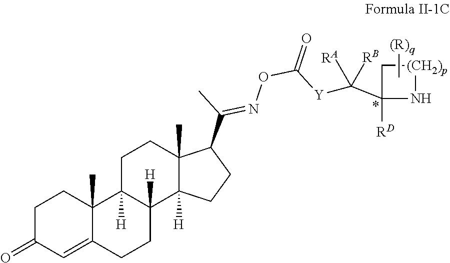

wherein Y, R^{A}, R^{B}, R^{D}, p, q, and R are the same as those described above for Formula IIC.

In some embodiments, the carbon atom labeled by the “*” sign is in a S configuration. In some embodiments, the carbon atom labeled by the “*” sign is in a R configuration.

In some embodiments, the

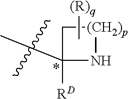

moiety in Formula II-1C is selected from the following:

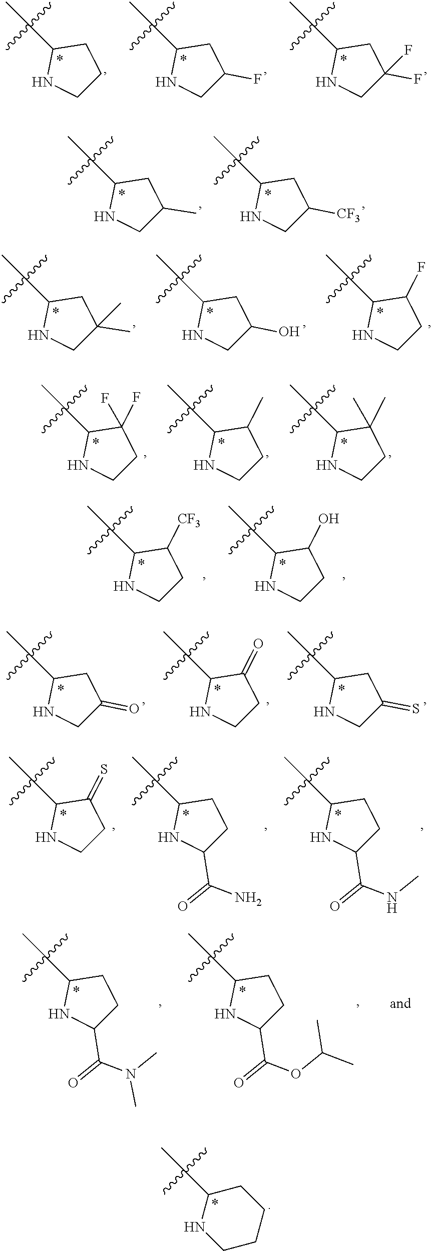

In some embodiments, the carbon atom labeled by the “*” sign is in a S configuration. In some embodiments, the carbon atom labeled by the “*” sign is in a R configuration.

In some embodiments, Y is NR³, and the

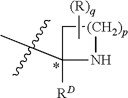

moiety in Formula II-1C is selected from the following:

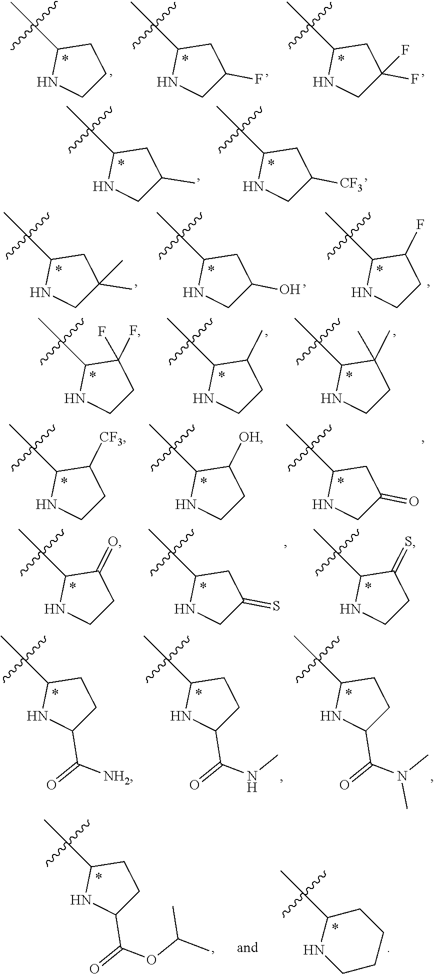

In some embodiments, the carbon atom labeled by the “*” sign is in a S configuration. In some embodiments, the carbon atom labeled by the “*” sign is in a R configuration. In some embodiments, R^{A} and R^{B} are hydrogen. In some embodiments, R^{A} is methyl, and R^{B} is hydrogen. In some embodiments, R^{A} and R^{B} are methyl. In some embodiments, R^{D} is hydrogen. In some embodiments, R^{D} is methyl. In some embodiments, R^{A}, R^{B}, and R^{D} are hydrogen. In some embodiments, R³ is hydrogen. In some embodiments, R³ is optionally substituted alkyl. In some embodiments, R³ is optionally substituted C₁-C₄ alkyl. In some embodiments, R³ is selected from —CH₃, —CF₃, CH₂CH₃, —CH₂CH₂OH, —CH₂CH₂F, —CH₂CF₃, —CH₂OCF₃, —CH₂CH₂OCF₃, —CH(CH₃)₂, —CH₂COOH, —CH₂CH₂COOH, —CH₂CH(CH₃)₂, —C(CH₃)₃, —CH₂CH₂NH₂, —CH₂CH₂NHCH₃, —CH₂CH₂N(CH₃)₂, and —CH₂CH₂N(CH₃)₃⁺. In some embodiments, R³ is —CH₃. In some embodiments, R³ is —CF₃. In some embodiments, R³ is —CH₂CH₃. In some embodiments, R³ is —CH₂CH₂OH. In some embodiments, R³ is —CH₂CH₂F. In some embodiments, R³ is —CH₂CF₃. In some embodiments, R³ is —CH₂OCF₃. In some embodiments, R³ is —CH₂CH₂OCF₃. In some embodiments, R³ is —CH(CH₃)₂. In some embodiments, R³ is —CH₂COOH. In some embodiments, R³ is —CH₂CH₂COOH. In some embodiments, R³ is —CH₂CH(CH₃)₂. In some embodiments, R³ is —C(CH₃)₃. In some embodiments, R³ is —CH₂CH₂NH₂. In some embodiments, R³ is —CH₂CH₂NHCH₃. In some embodiments, R³ is —CH₂CH₂N(CH₃)₂. In some embodiments, R³ is CH₂CH₂N(CH₃)₃⁺. In some embodiments, R³ is optionally substituted carbocyclyl or optionally substituted heterocyclyl. In some embodiments, R³ is selected from

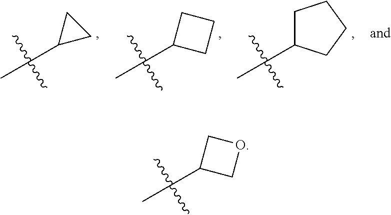

In some embodiments, R³ is

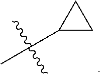 [SMILES: *C1CC1]

In some embodiments, R³ is

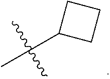 [SMILES: *C1CCC1]

In some embodiments, R³ is

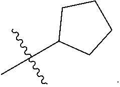 [SMILES: *C1CCCC1]

In some embodiments, R³ is

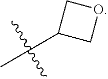 [SMILES: *C1COC1]

In some embodiments, Formula II-1 is in the form of Formula II-1D,

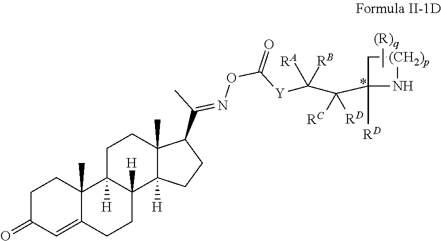

wherein Y, R^{A}, R^{B}, R^{C}, R^{D}, R^{F}, p, q, and R are the same as those described above for Formula IID.

In some embodiments, the carbon atom labeled by the “*” sign is in a S configuration. In some embodiments, the carbon atom labeled by the “*” sign is in a R configuration.

In some embodiments, the

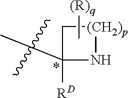

moiety in Formula II-1D is selected from the following:

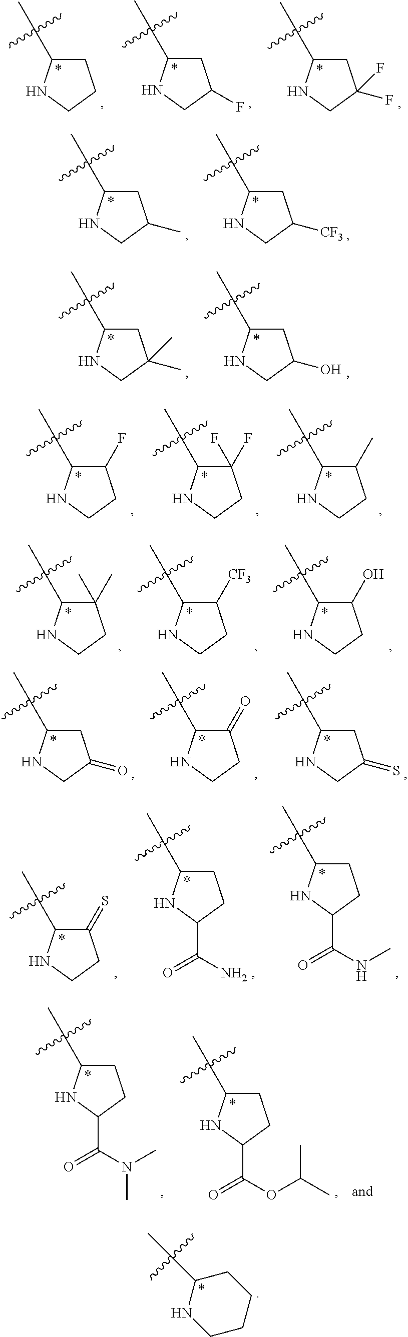

In some embodiments, the carbon atom labeled by the “*” sign is in a S configuration. In some embodiments, the carbon atom labeled by the “*” sign is in a R configuration.

In some embodiments, Y is NR³, and the

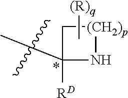

moiety in Formula II-1D is selected from the following:

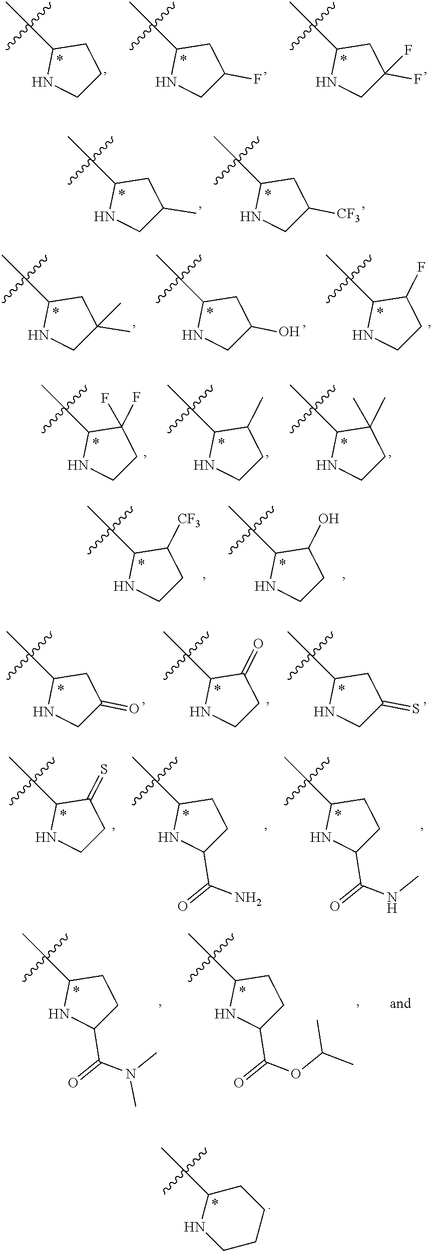

In some embodiments, the carbon atom labeled by the “*” sign is in a S configuration. In some embodiments, the carbon atom labeled by the “*” sign is in a R configuration. In some embodiments, R^{A} and R^{B} are hydrogen. In some embodiments, R^{A} is methyl, and R^{B} is hydrogen. In some embodiments, R^{A} and R^{B} are methyl. In some embodiments, R^{C} and R^{D} are hydrogen. In some embodiments, R^{C} is methyl, and R^{D} is hydrogen. In some embodiments, and R^{C} and R^{D} are methyl. In some embodiments, R^{F} is hydrogen. In some embodiments, R^{F} is methyl. In some embodiments, R^{A}, R^{B}, R^{C}, R^{D}, and R^{F} are hydrogen. In some embodiments, R³ is hydrogen. In some embodiments, R³ is optionally substituted alkyl. In some embodiments, R³ is optionally substituted C₁-C₄ alkyl. In some embodiments, R³ is selected from —CH₃, —CF₃, —CH₂CH₃, CH₂CH₂OH, —CH₂CH₂F, —CH₂CF₃, CH₂OCF₃, —CH₂CH₂OCF₃, —CH(CH₃)₂, —CH₂COOH, —CH₂CH₂COOH, —CH₂CH(CH₃)₂, —C(CH₃)₃, CH₂CH₂NH₂, —CH₂CH₂NHCH₃, CH₂CH₂N(CH₃)₂, and —CH₂CH₂N(CH₃)₃⁺. In some embodiments, R³ is —CH₃. In some embodiments, R³ is —CF₃. In some embodiments, R³ is —CH₂CH₃. In some embodiments, R³ is —CH₂CH₂OH. In some embodiments, R³ is —CH₂CH₂F. In some embodiments, R³ is —CH₂CF₃. In some embodiments, R³ is —CH₂OCF₃. In some embodiments, R³ is —CH₂CH₂OCF₃. In some embodiments, R³ is —CH(CH₃)₂. In some embodiments, R³ is —CH₂COOH. In some embodiments, R³ is —CH₂CH₂COOH. In some embodiments, R³ is —CH₂CH(CH₃)₂. In some embodiments, R³ is C(CH₃)₃. In some embodiments, R³ is —CH₂CH₂NH₂. In some embodiments, R³ is —CH₂CH₂NHCH₃. In some embodiments, R³ is —CH₂CH₂N(CH₃)₂. In some embodiments, R³ is —CH₂CH₂N(CH₃)₃⁺. In some embodiments, R³ is optionally substituted carbocyclyl or optionally substituted heterocyclyl. In some embodiments, R³ is selected from

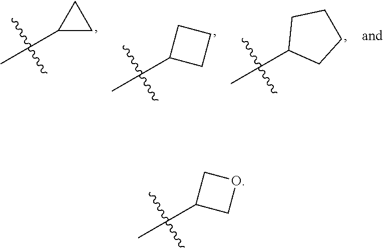

In some embodiments, R³ is

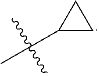 [SMILES: *C1CC1]

In some embodiments, R³ is

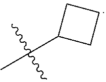 [SMILES: *C1CCC1]

In some embodiments, R³ is

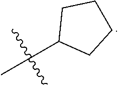 [SMILES: *C1CCCC1]

In some embodiments, R³ is

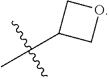 [SMILES: *C1COC1]

Exemplary compounds of Formula II-1 include:

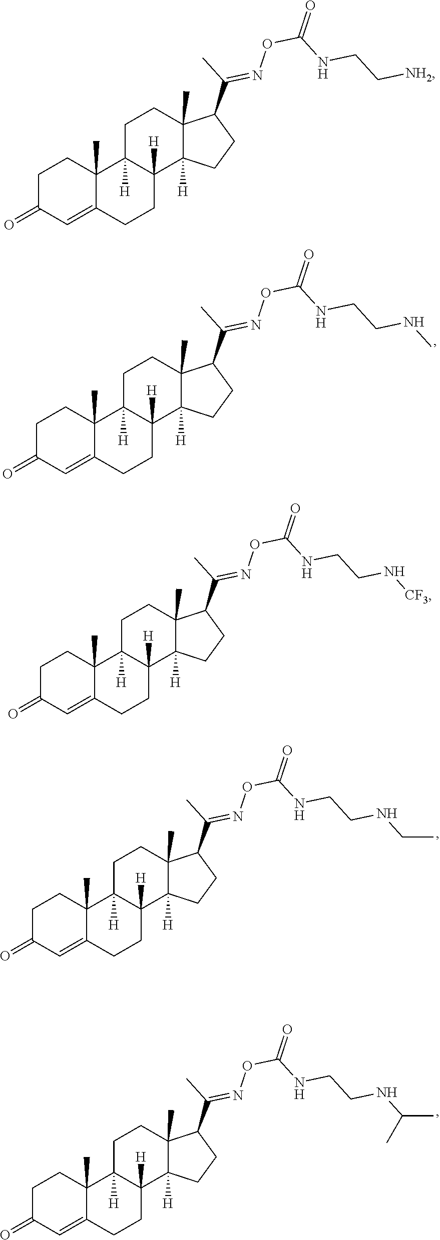

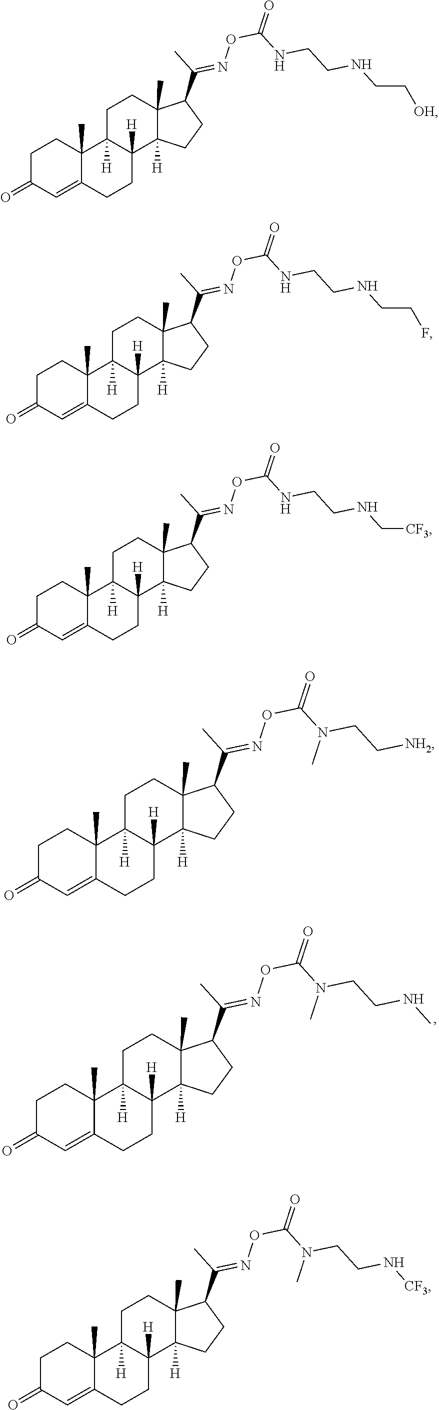

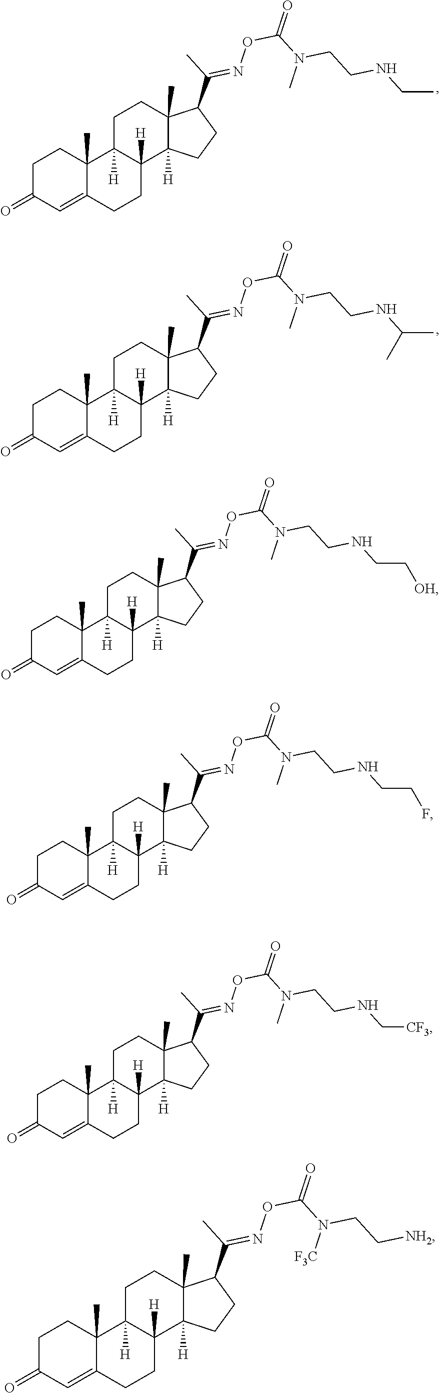

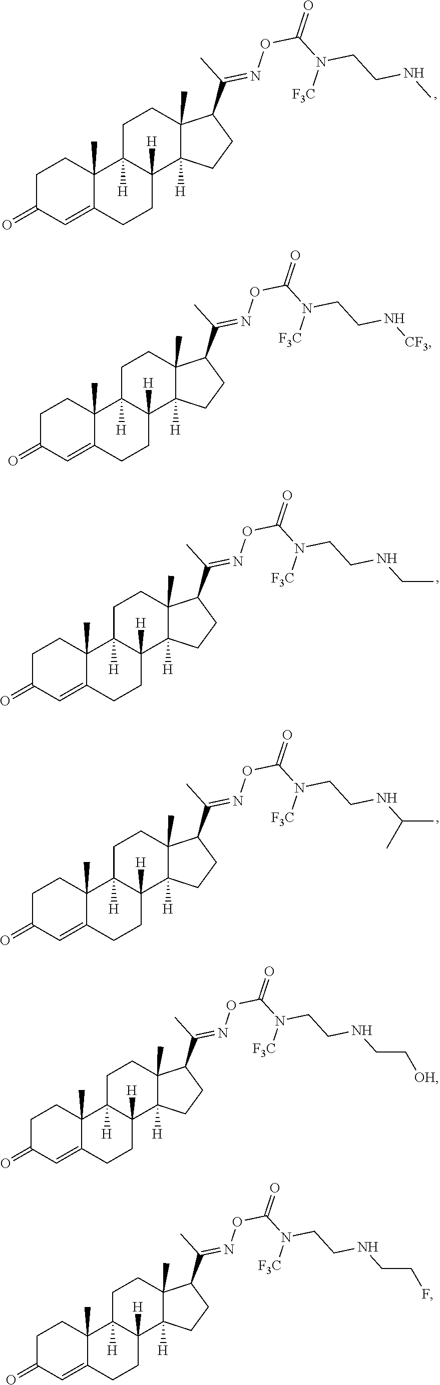

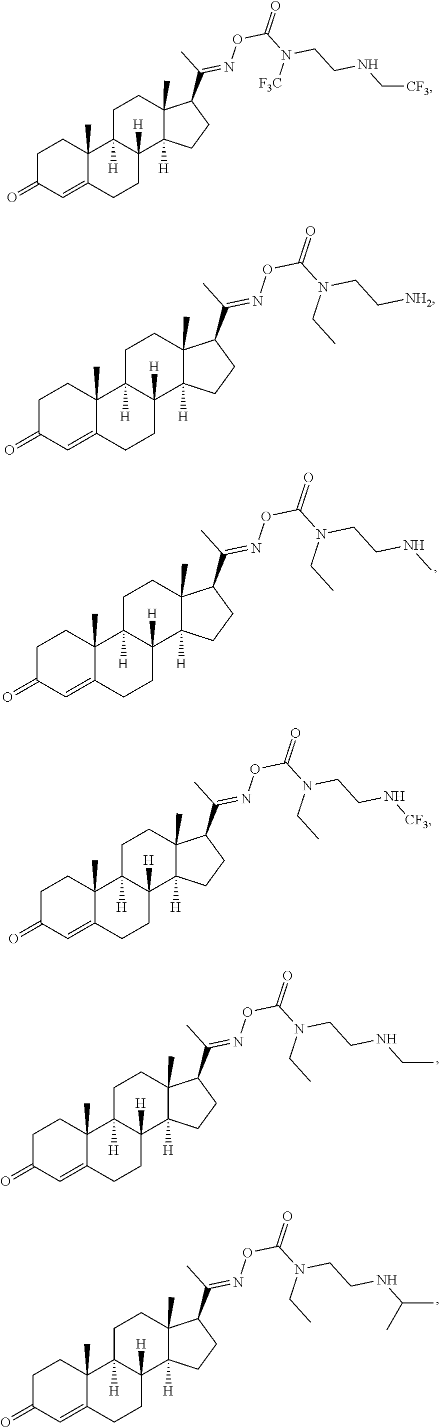

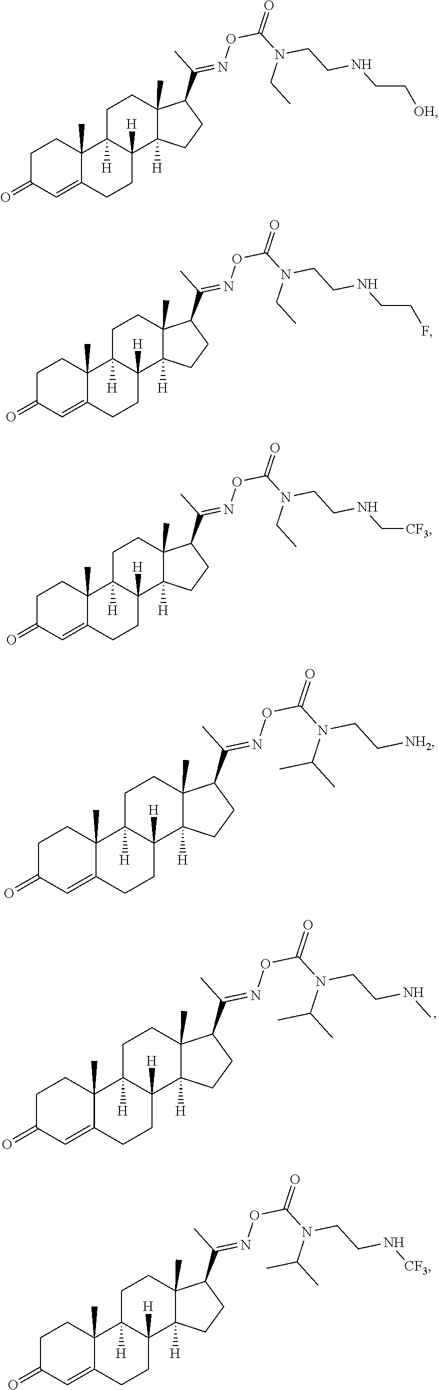

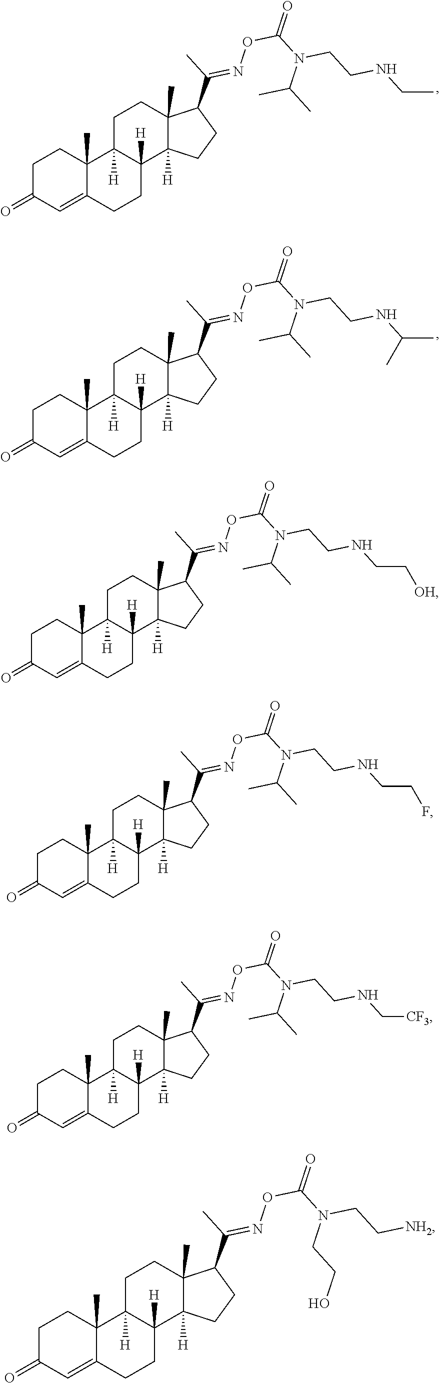

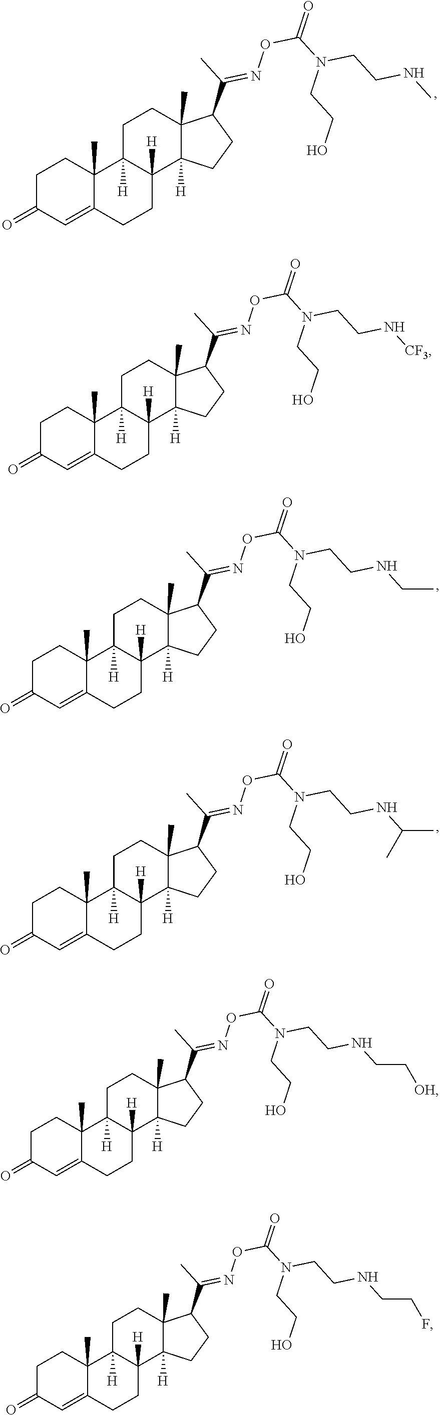

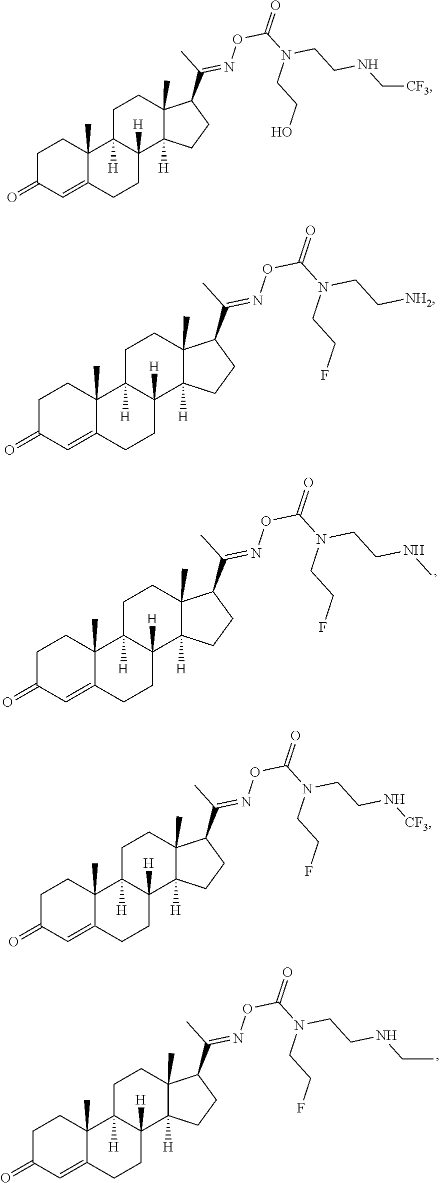

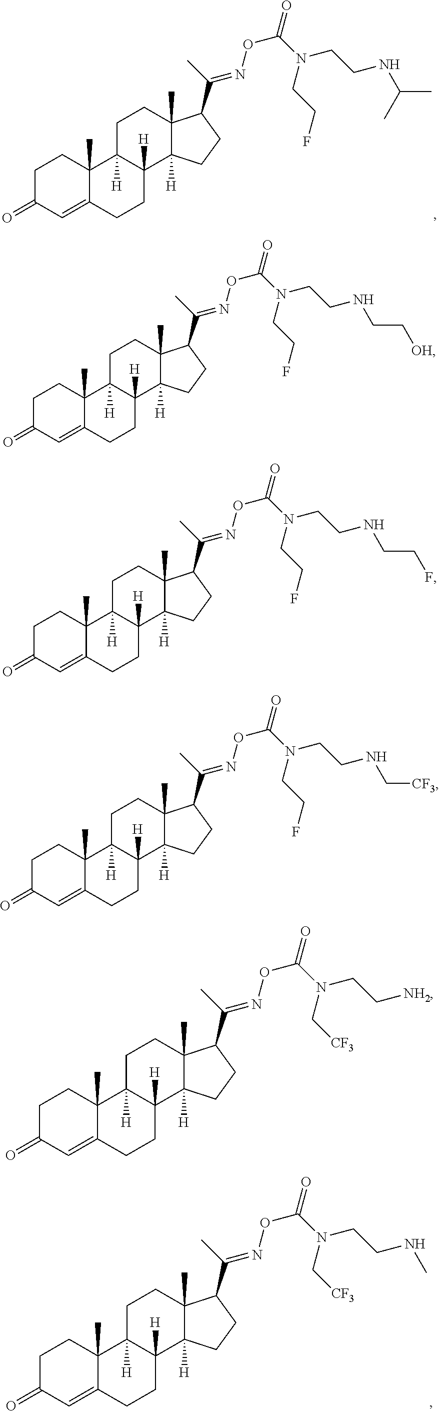

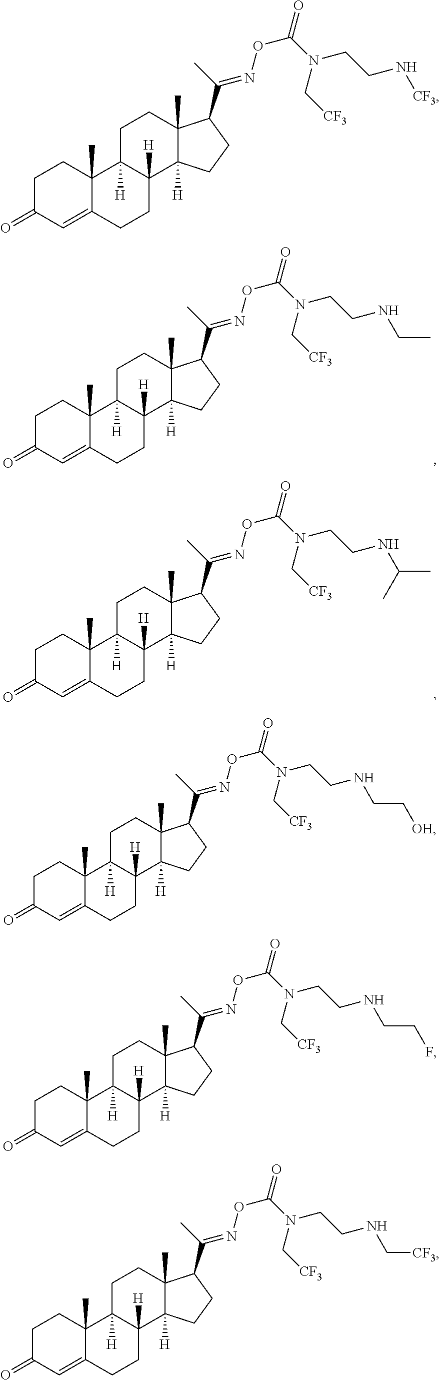

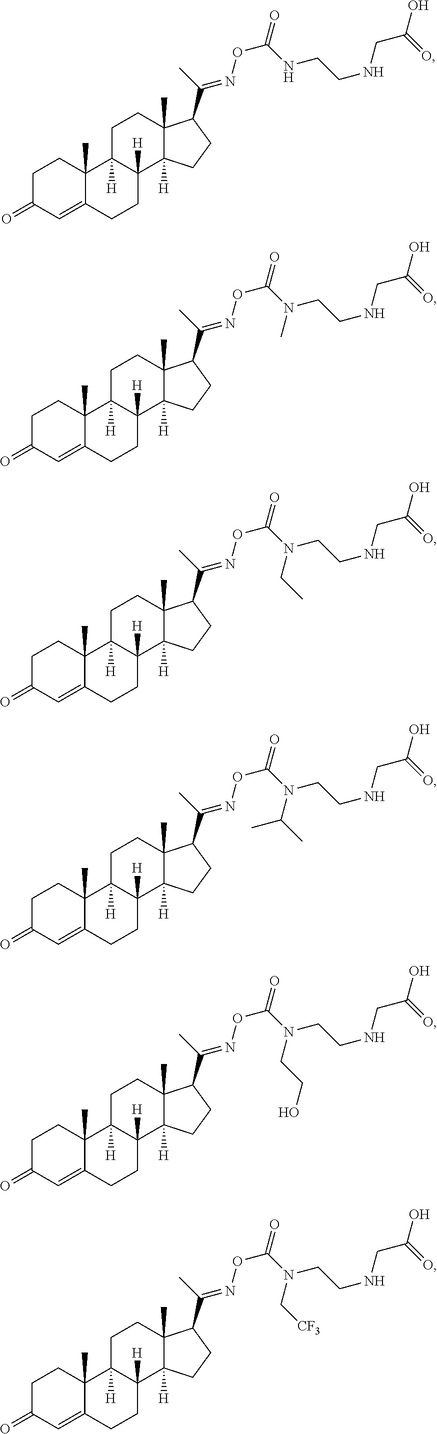

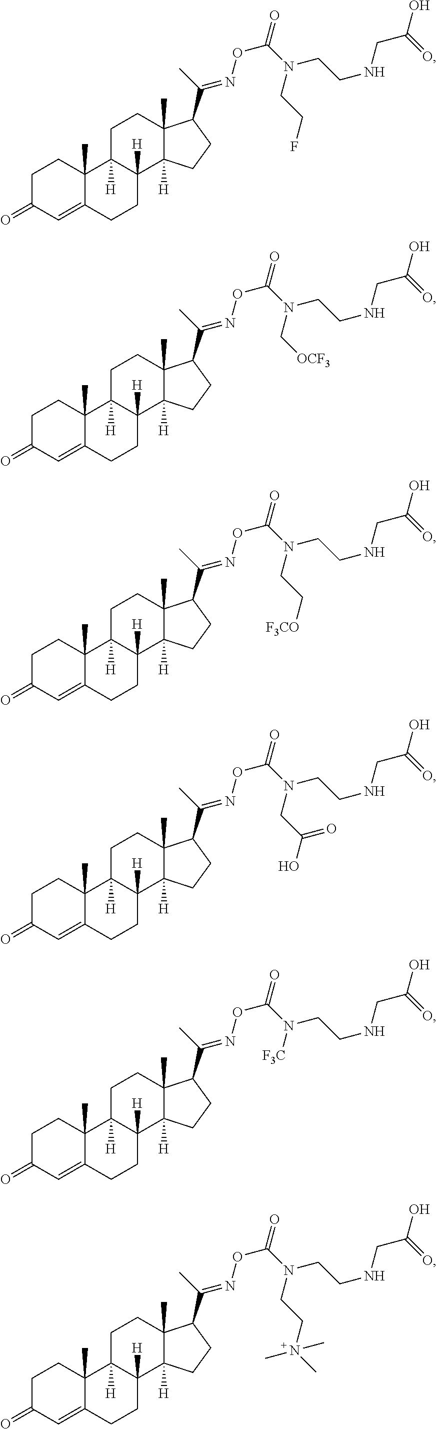

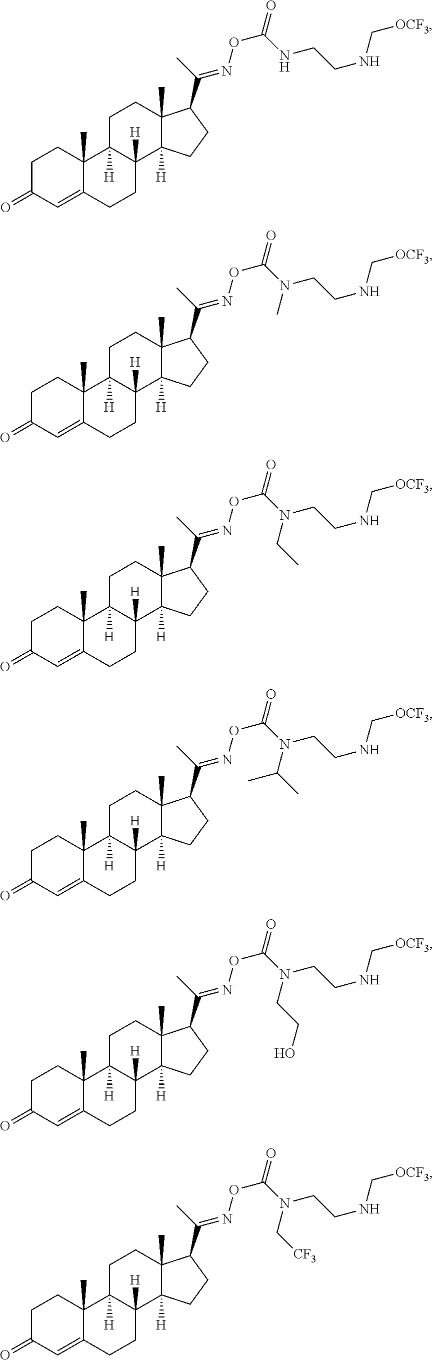

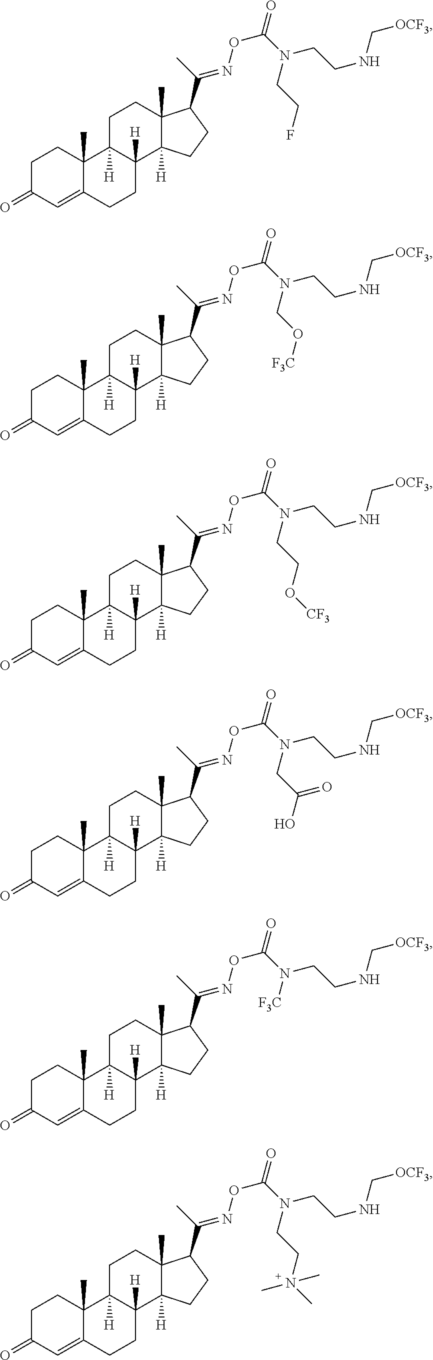

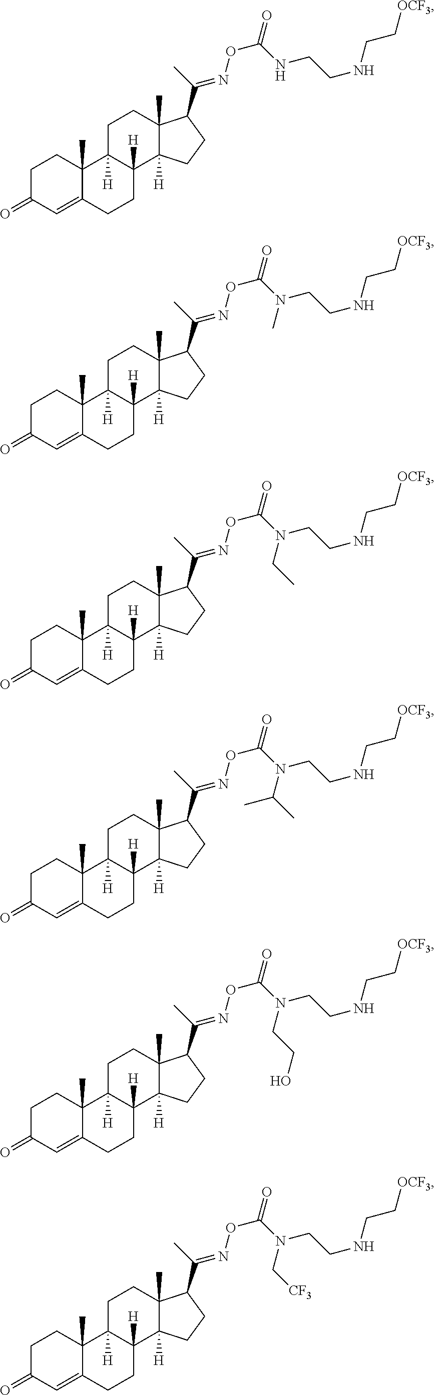

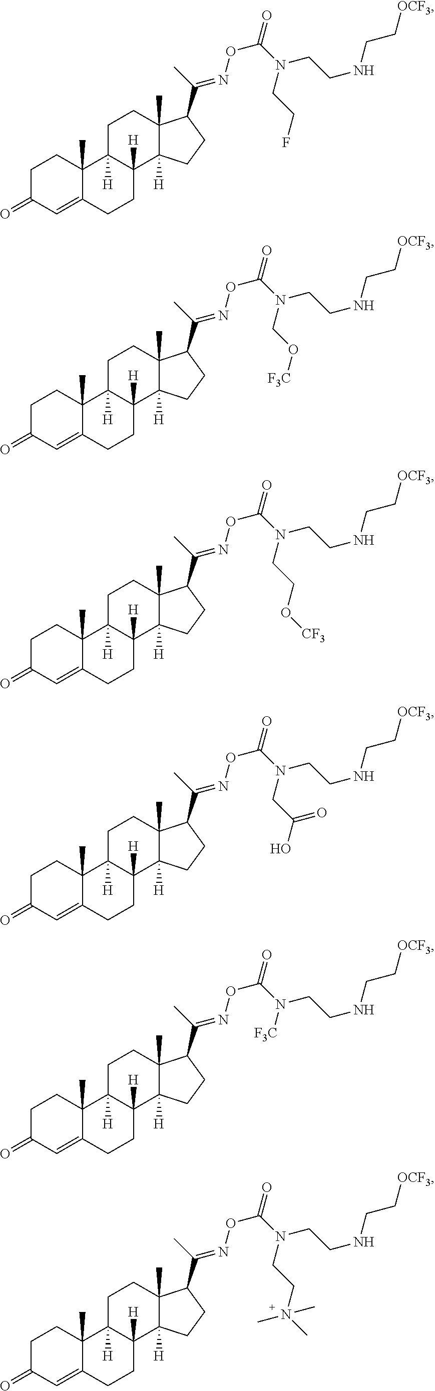

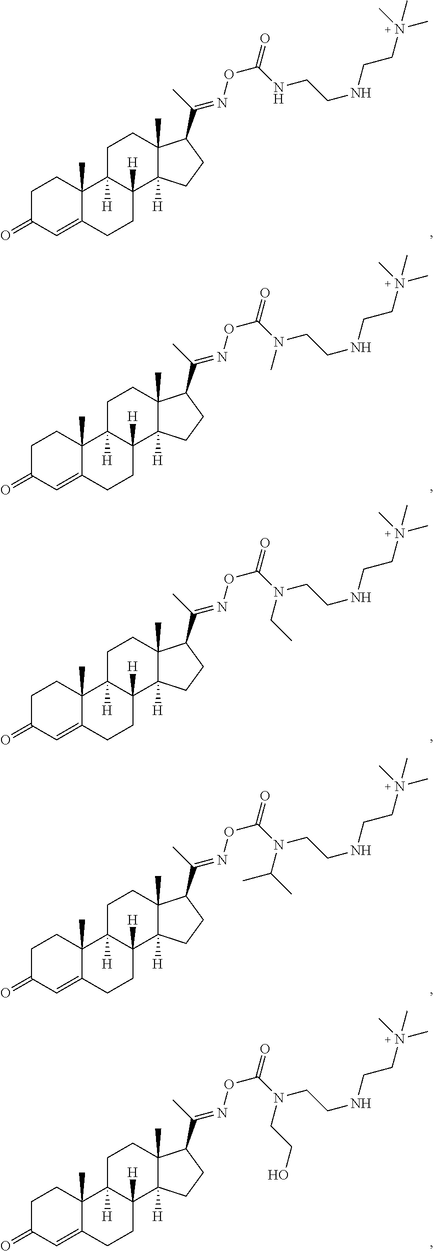

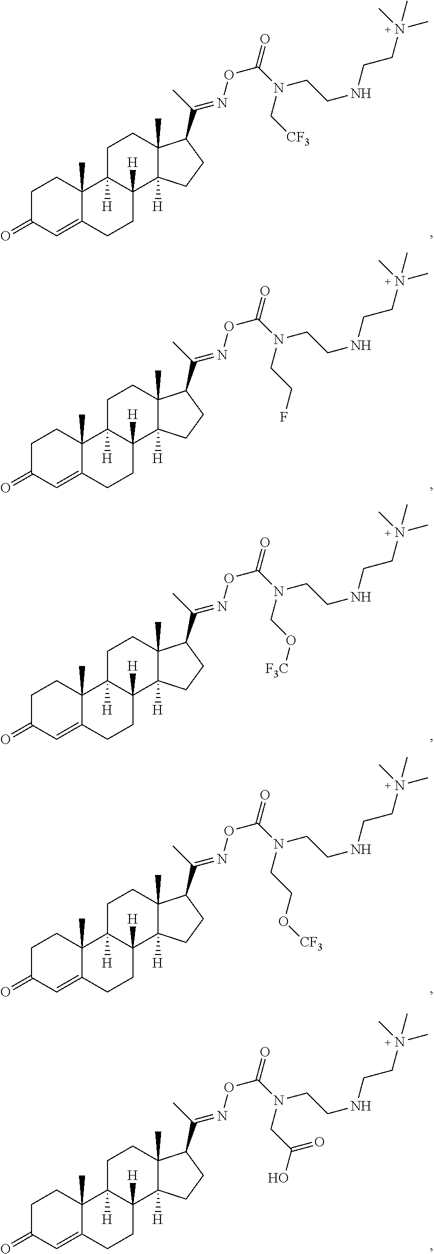

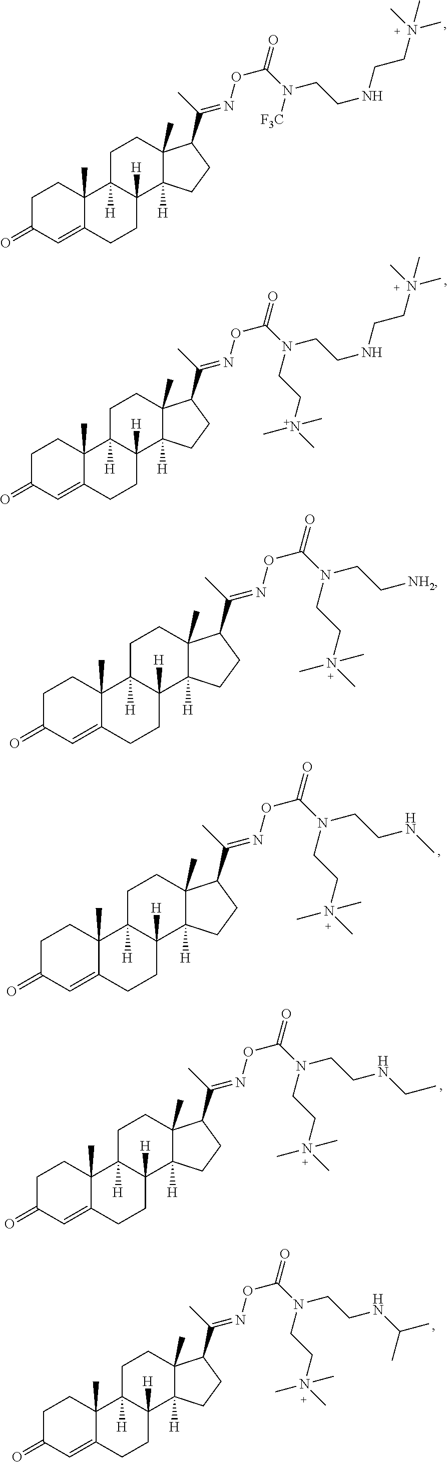

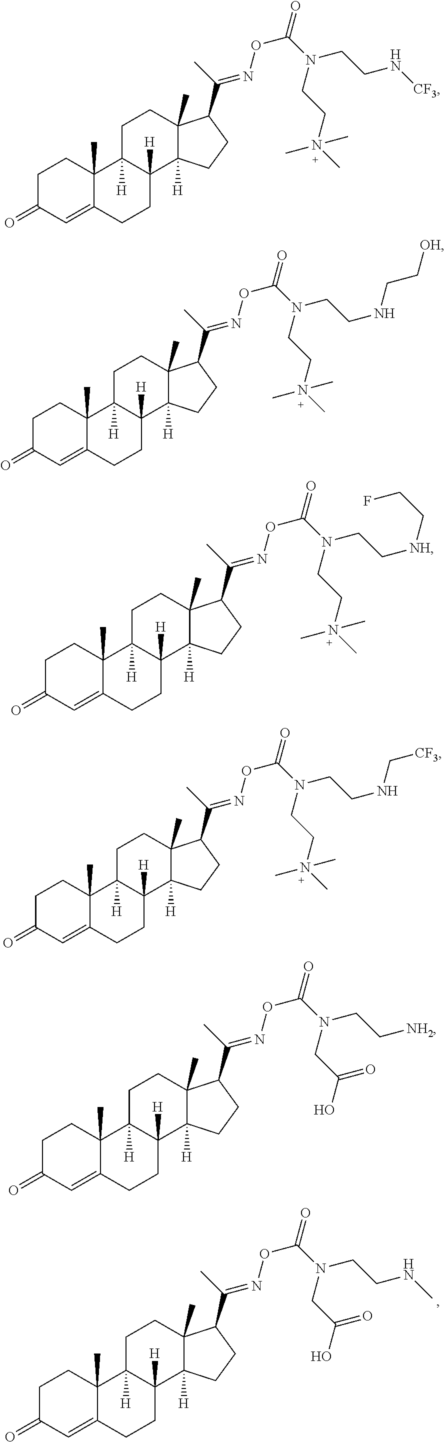

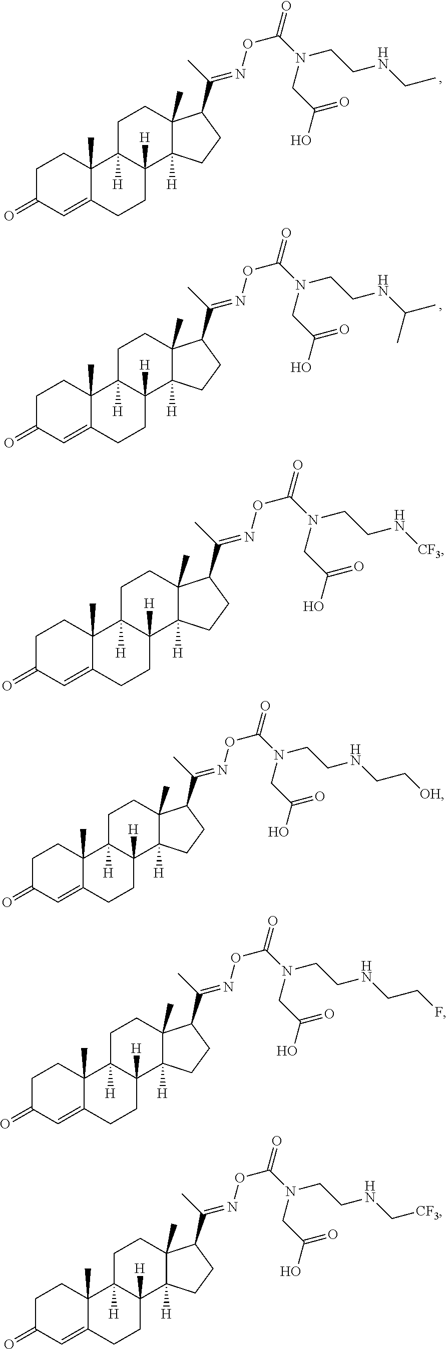

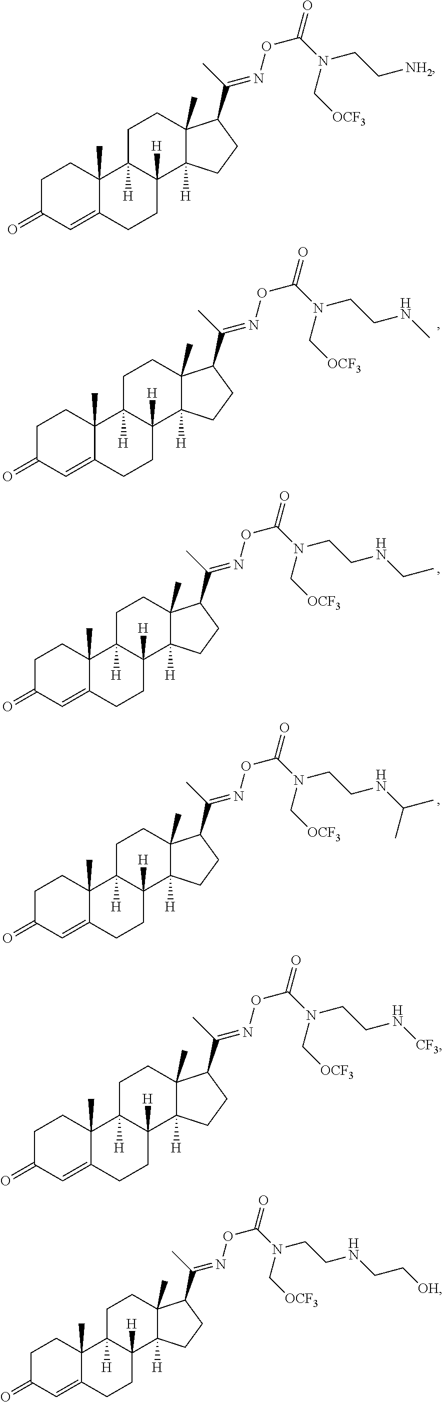

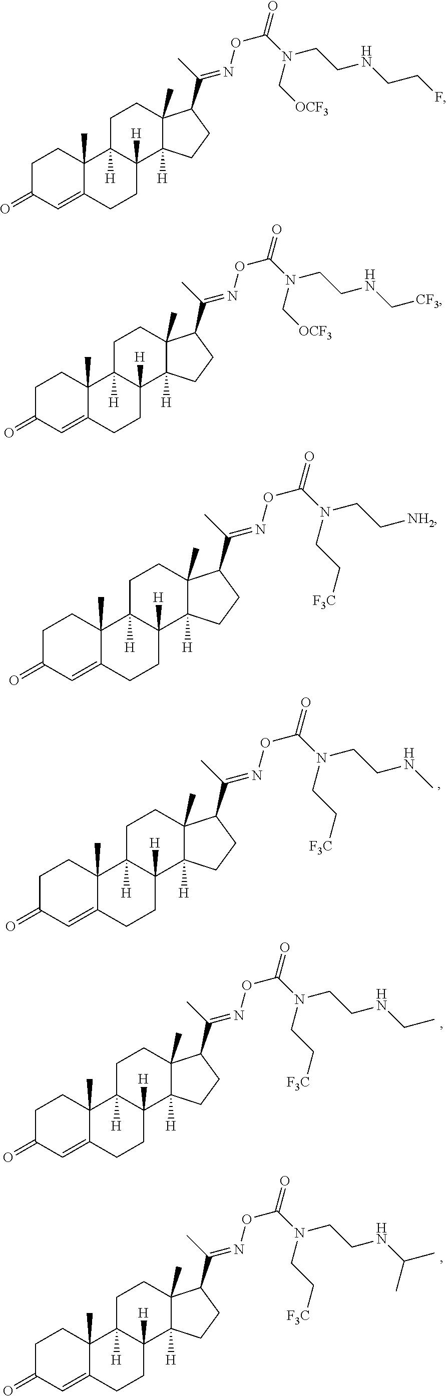

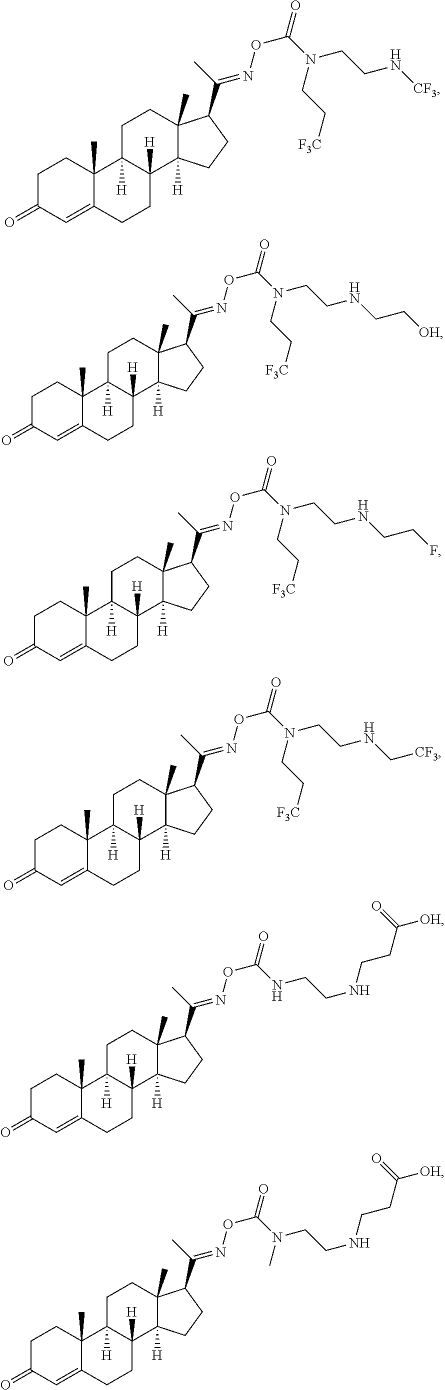

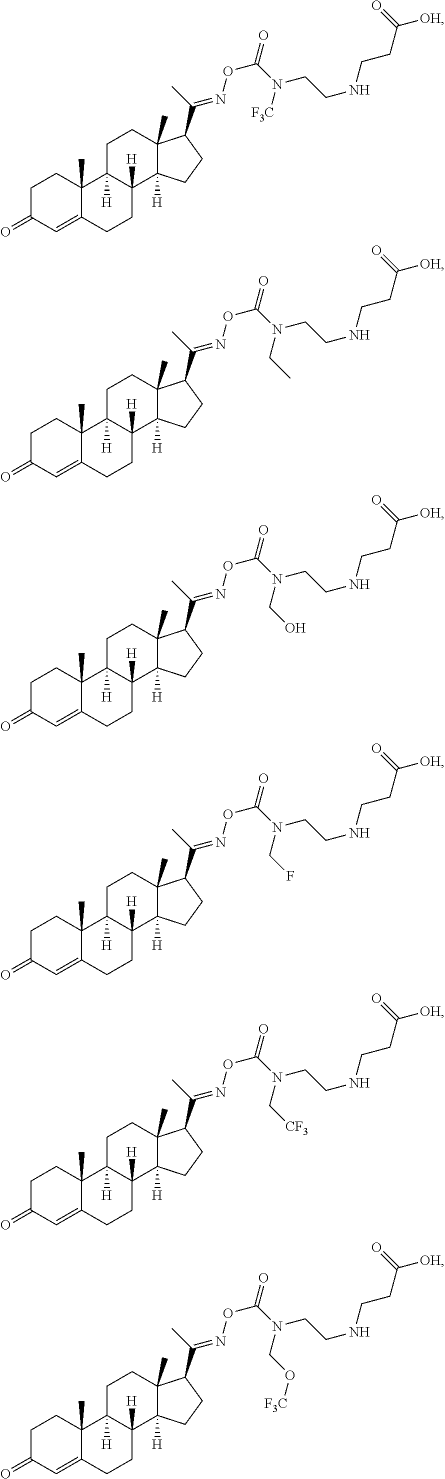

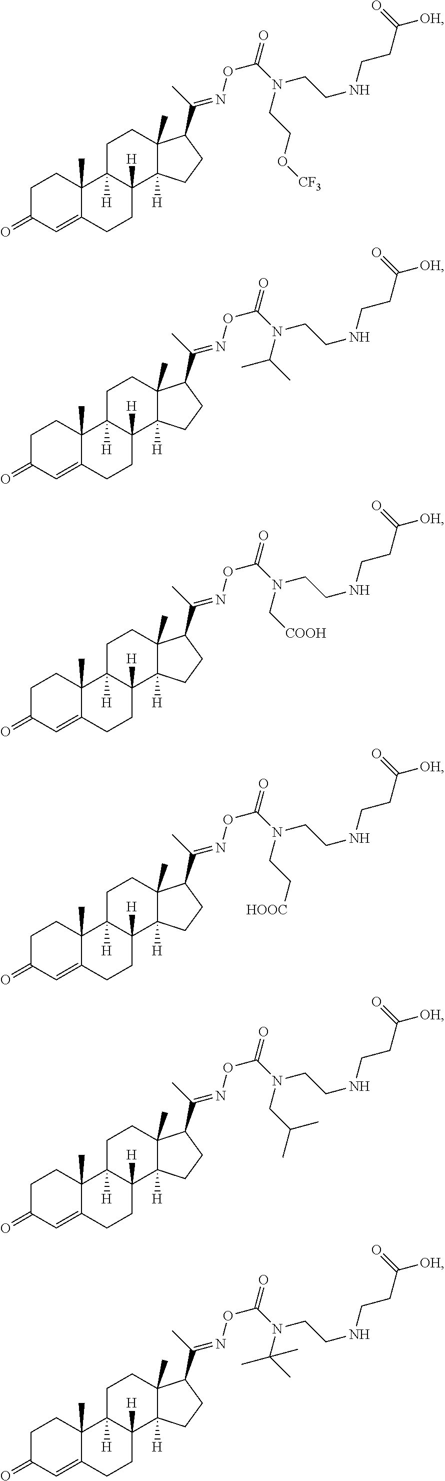

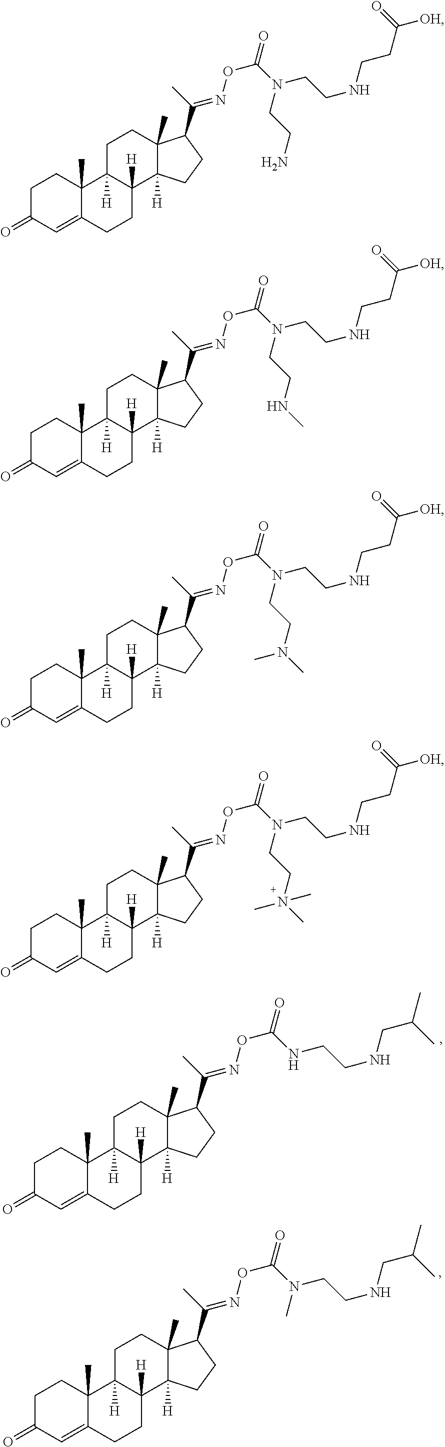

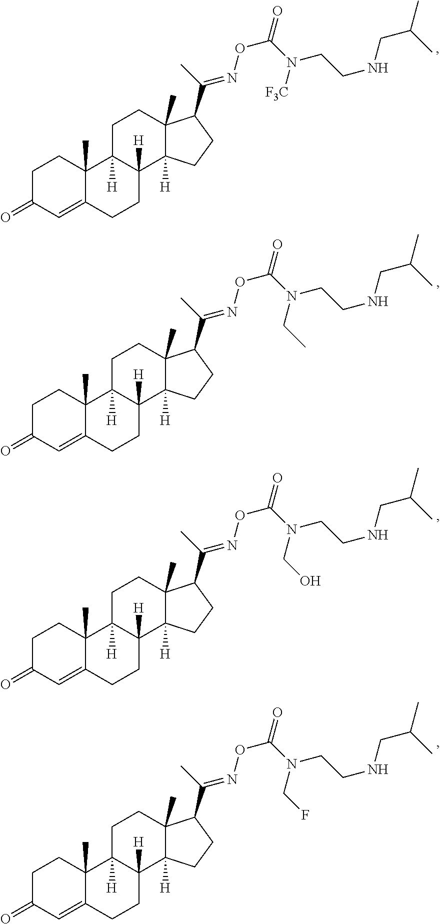

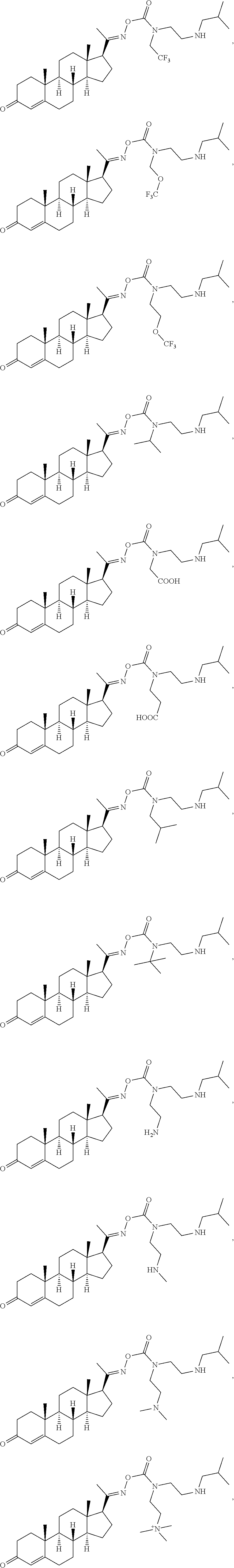

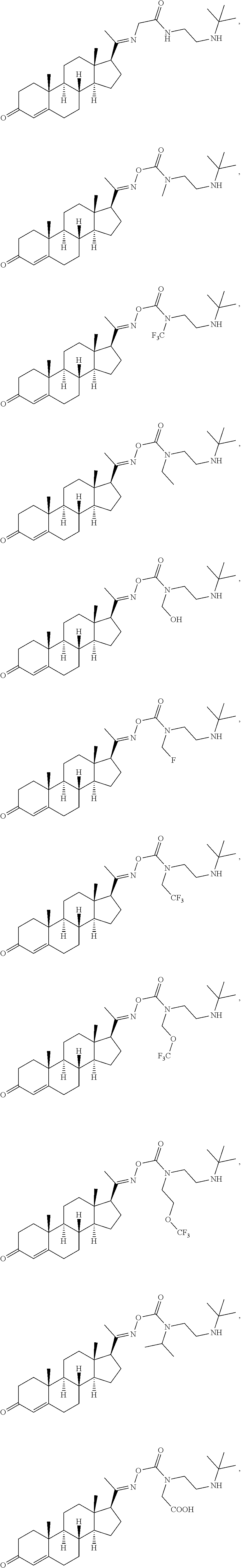

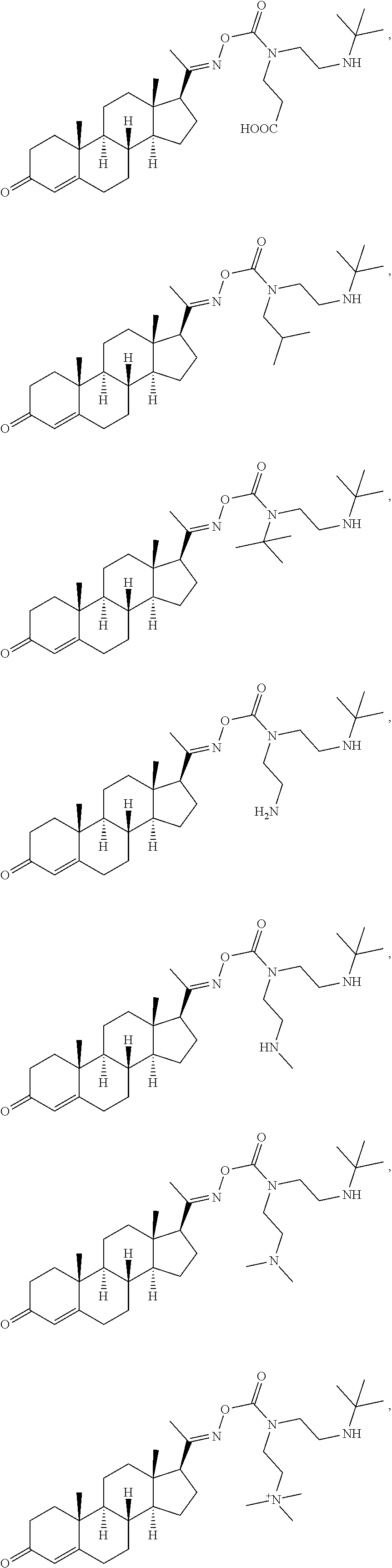

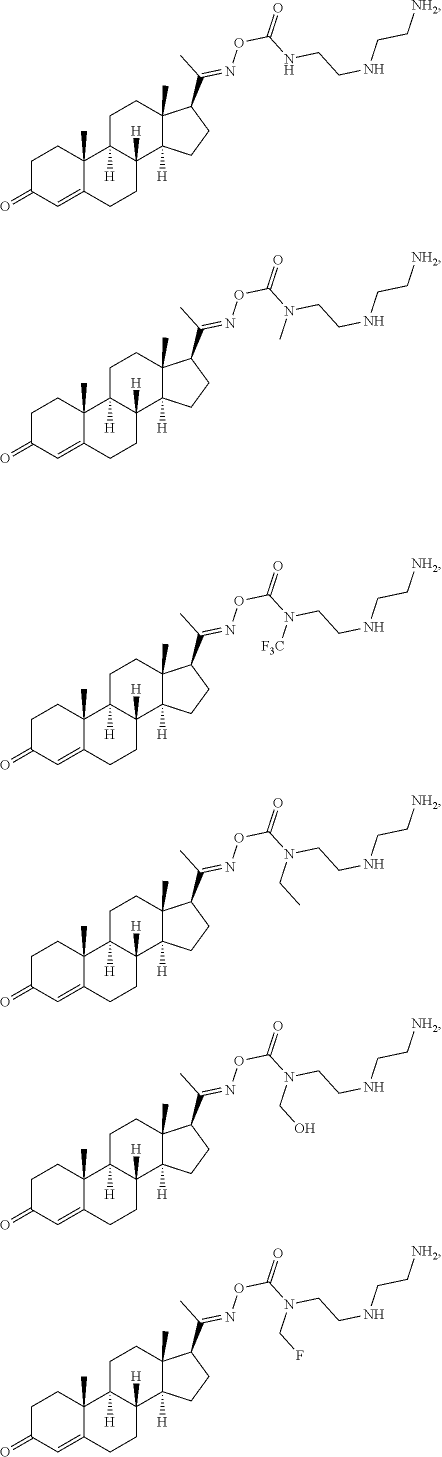

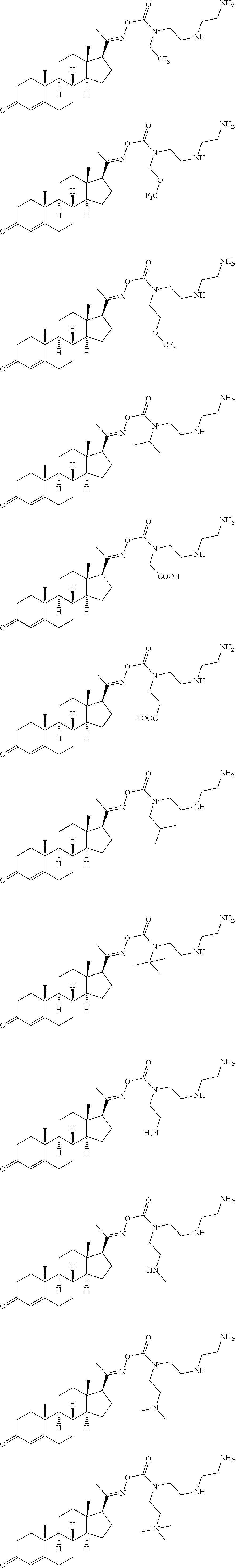

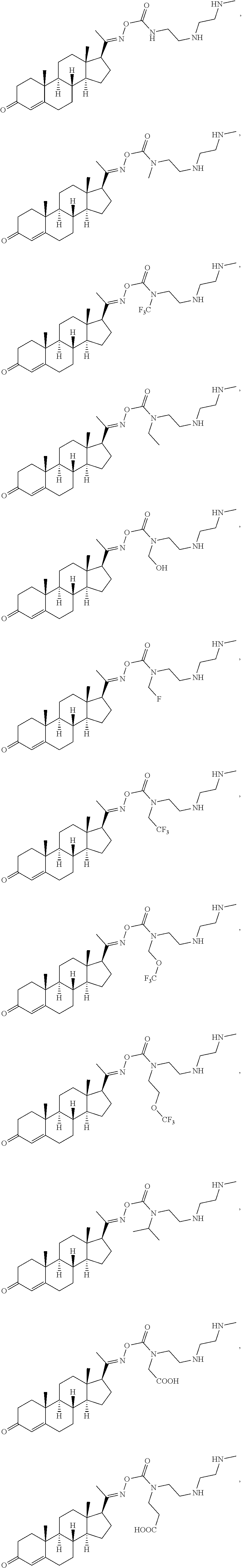

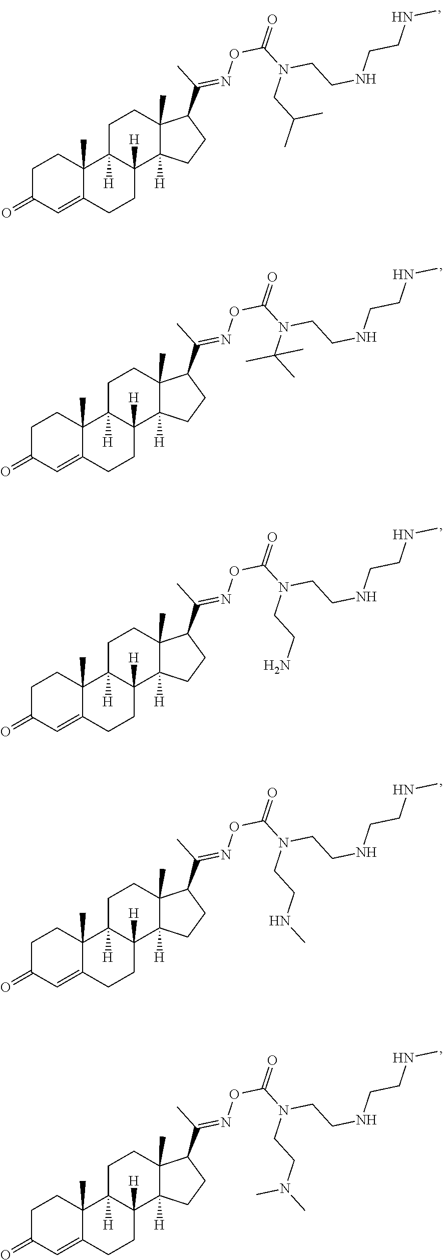

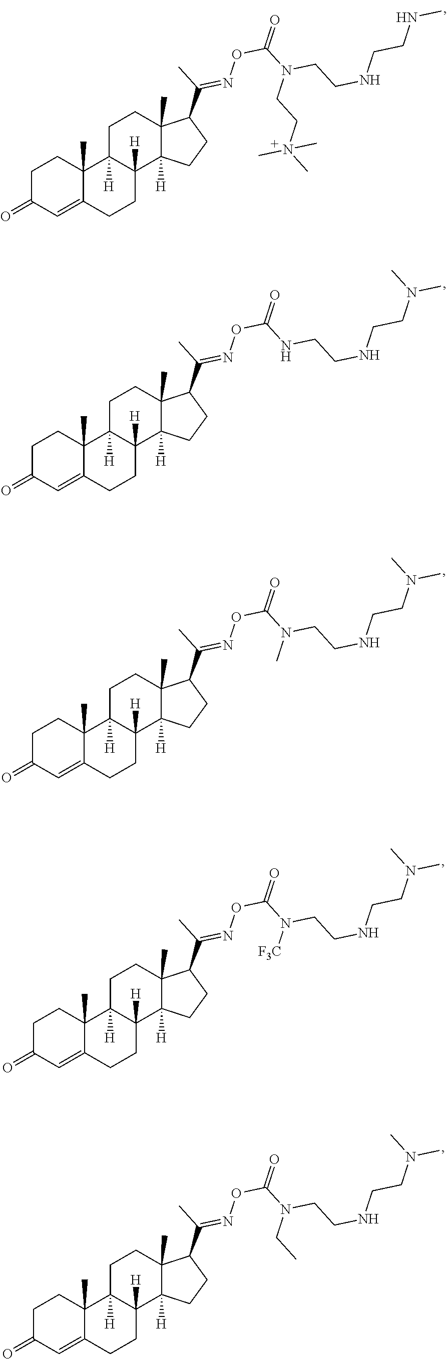

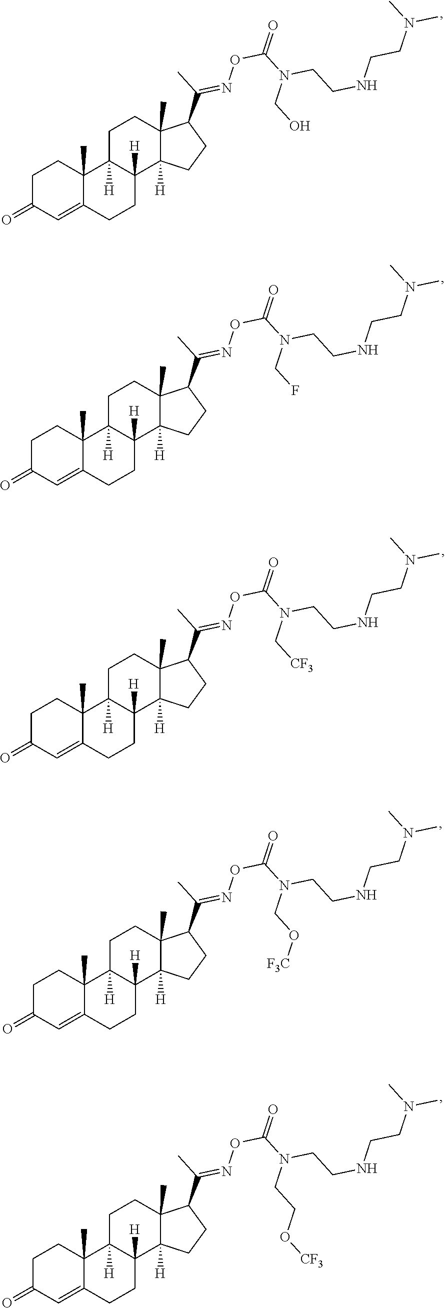

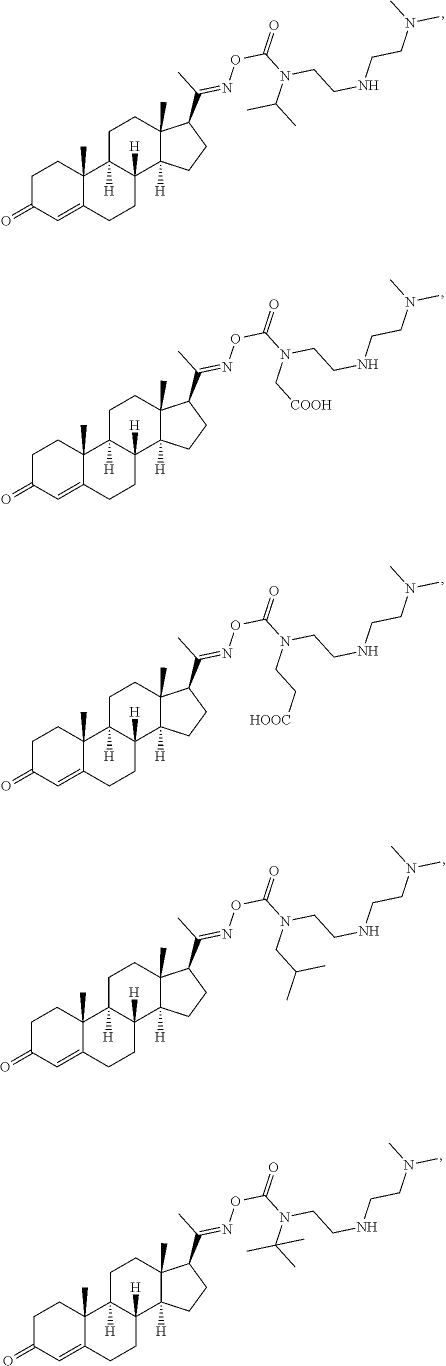

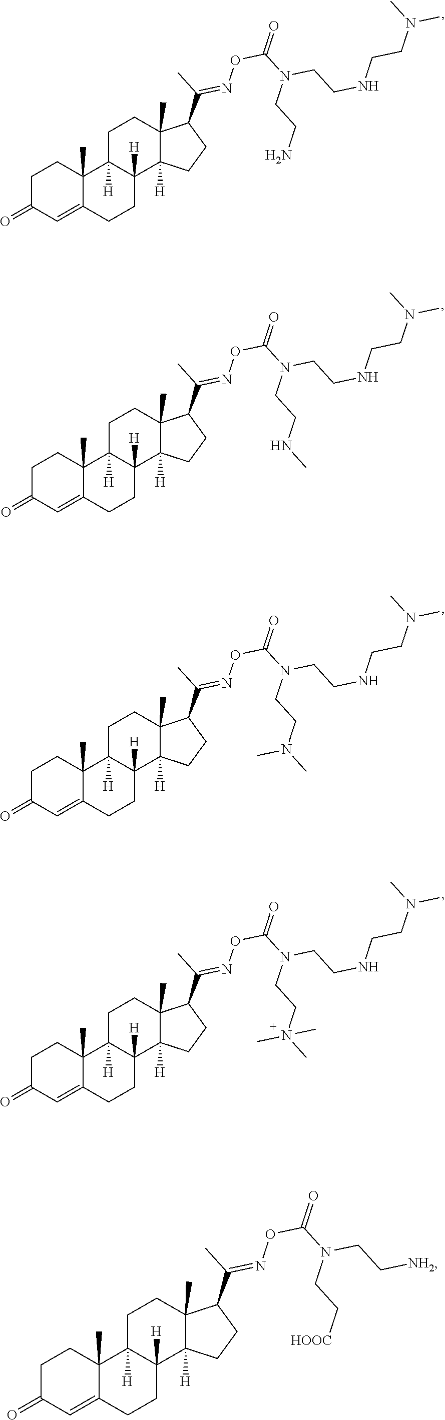

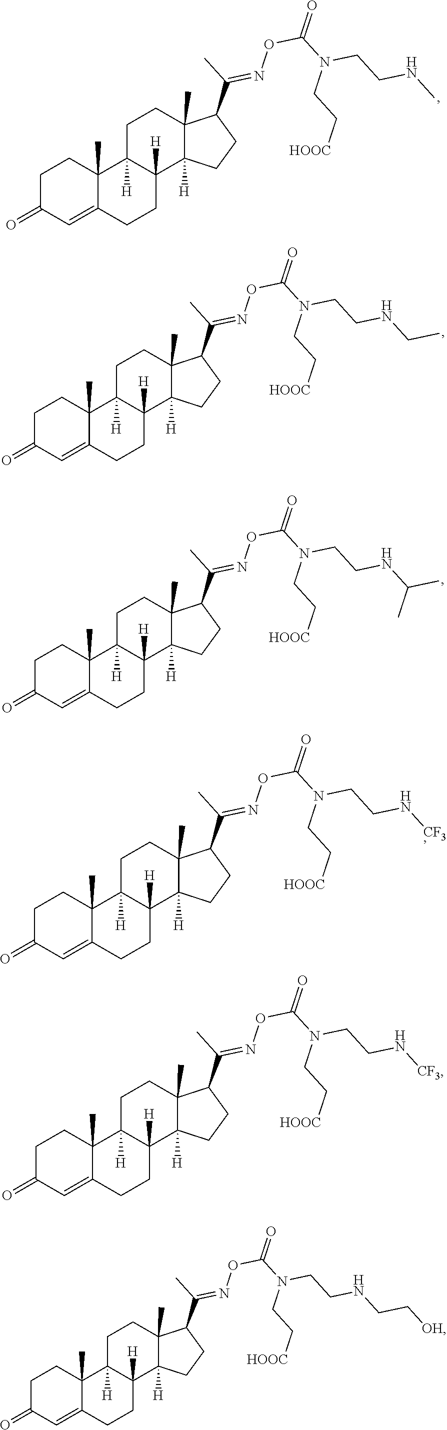

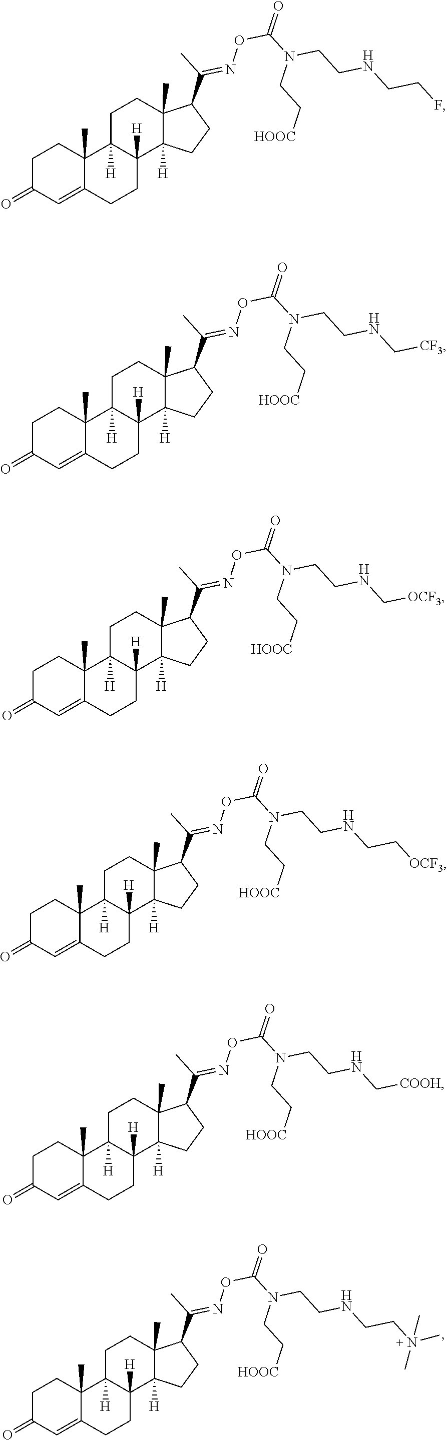

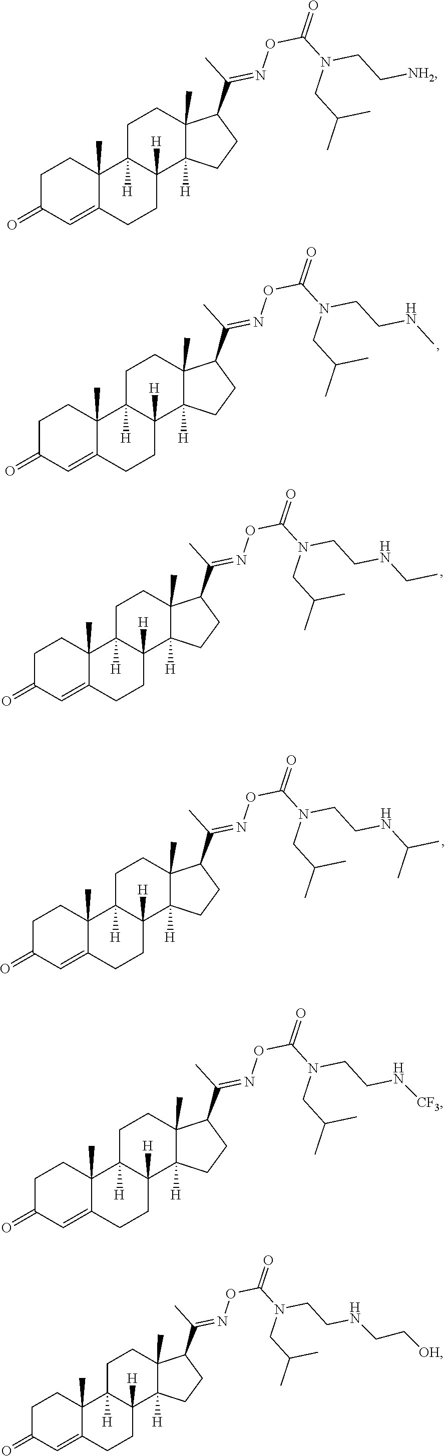

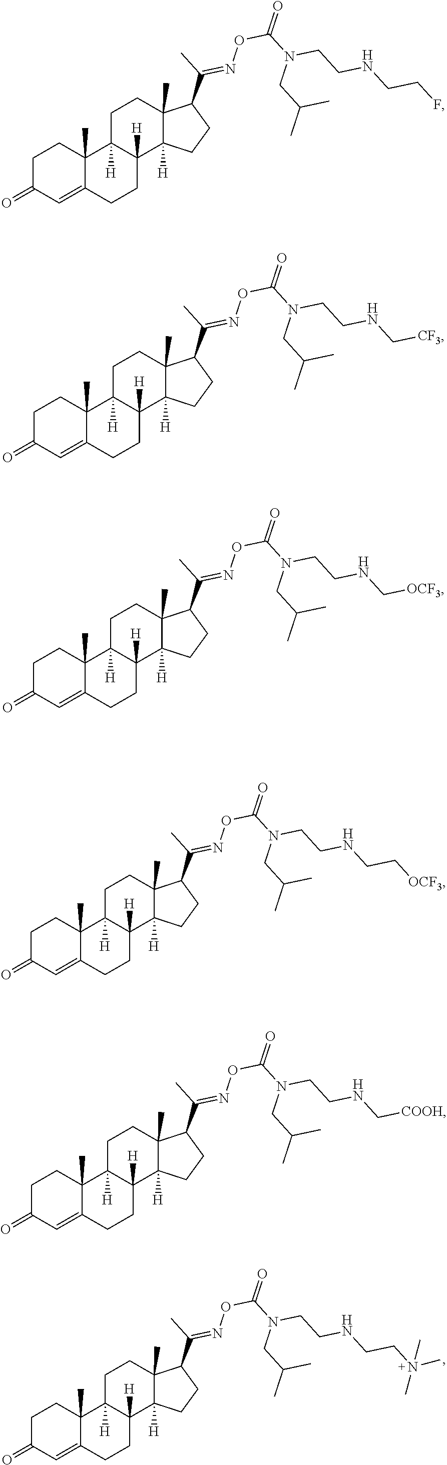

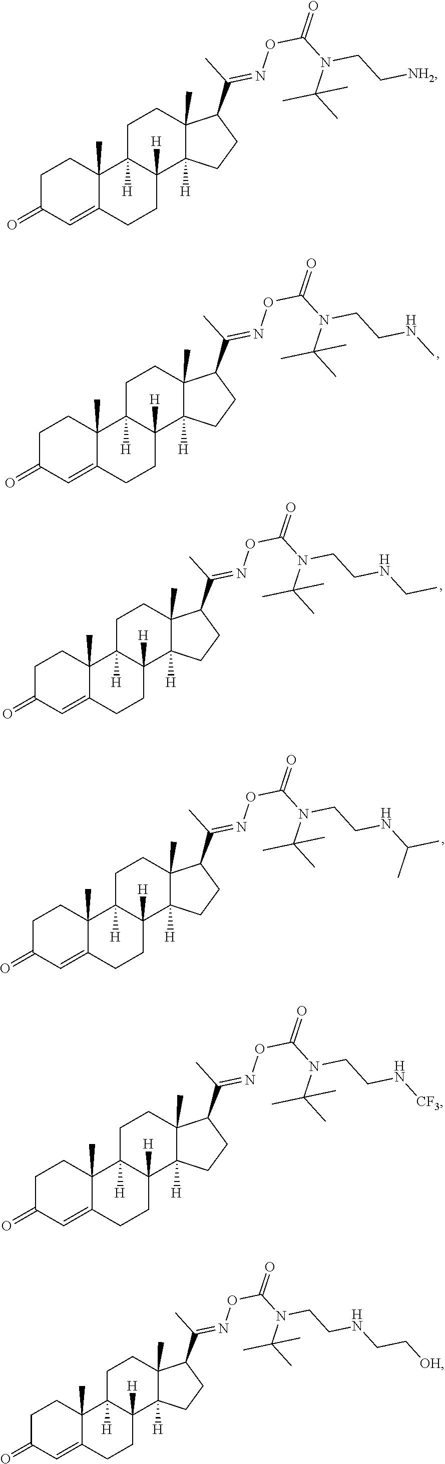

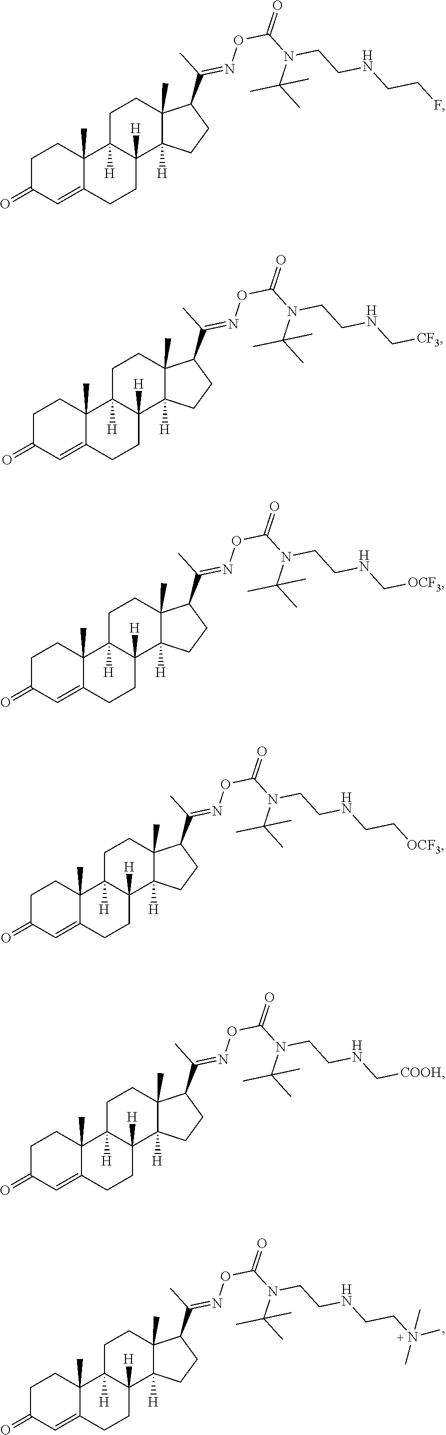

and pharmaceutically acceptable salts thereof.

Exemplary compounds of Formula II-1 also include:

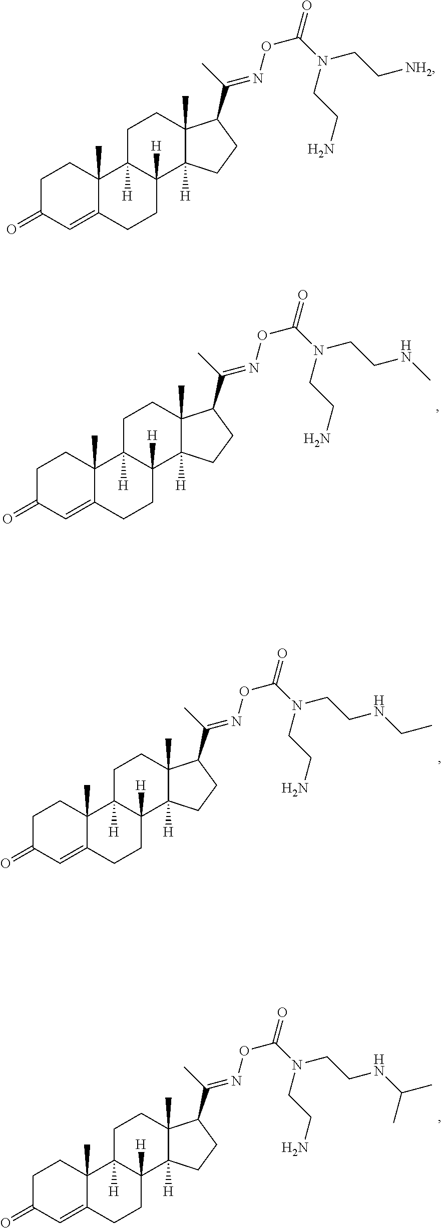

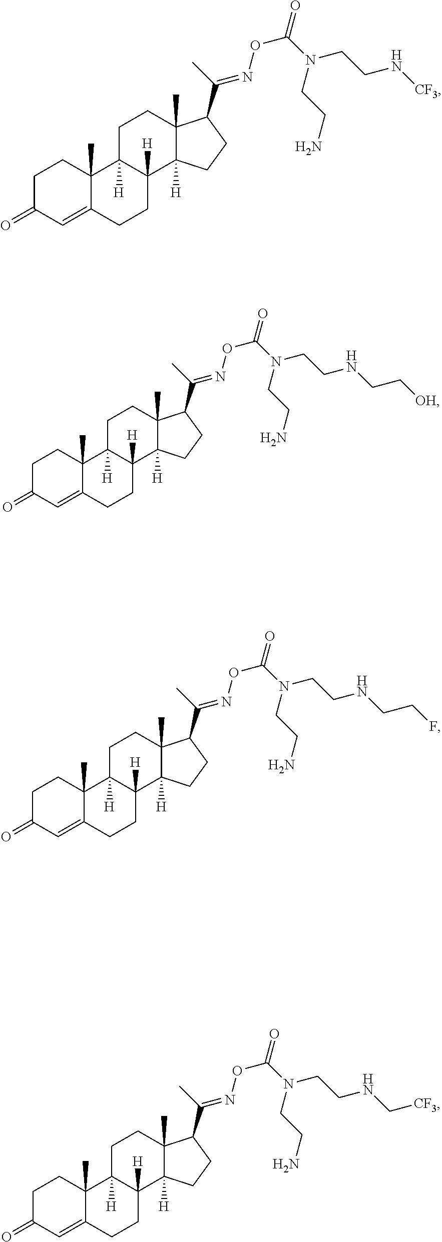

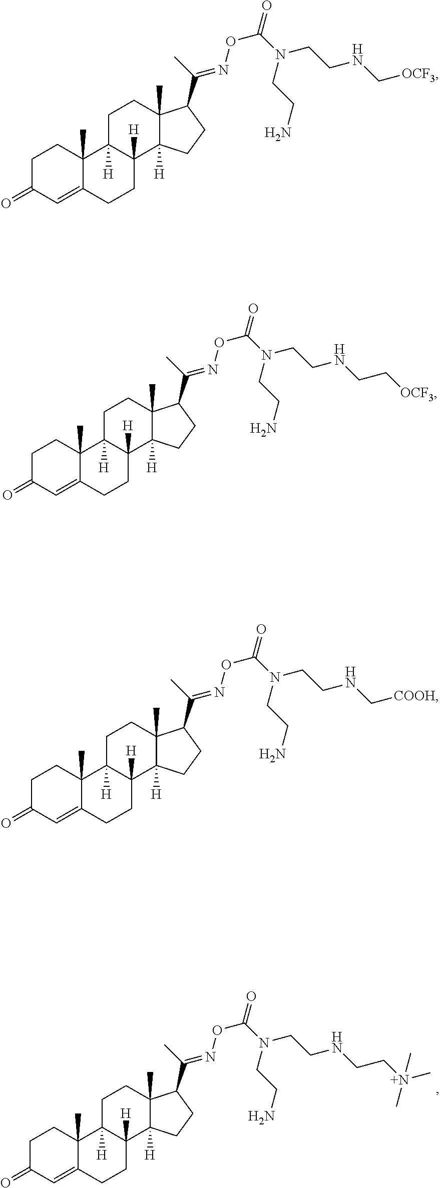

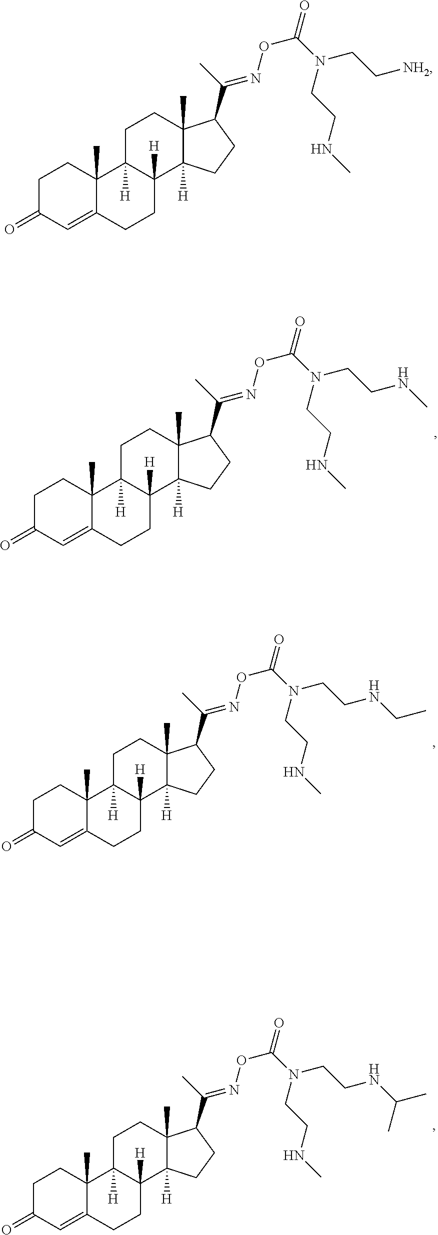

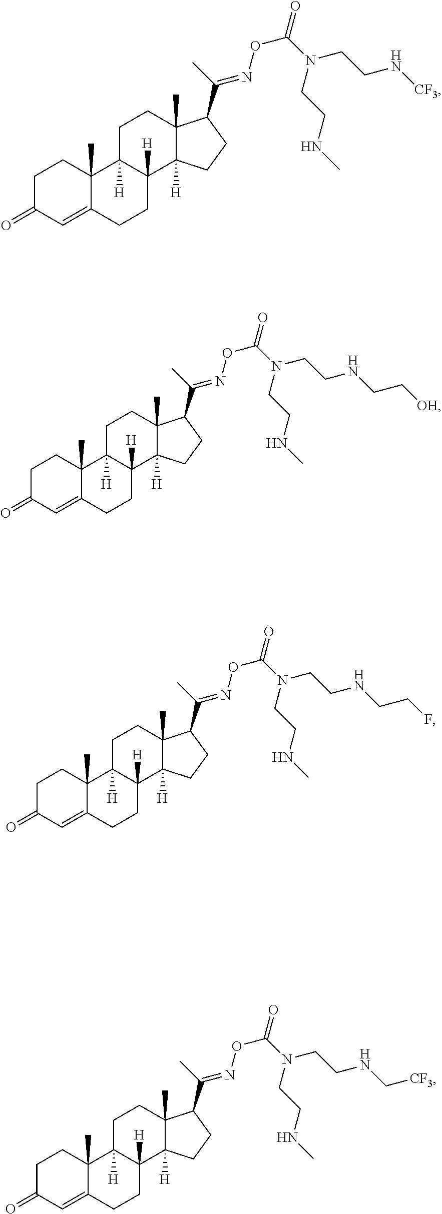

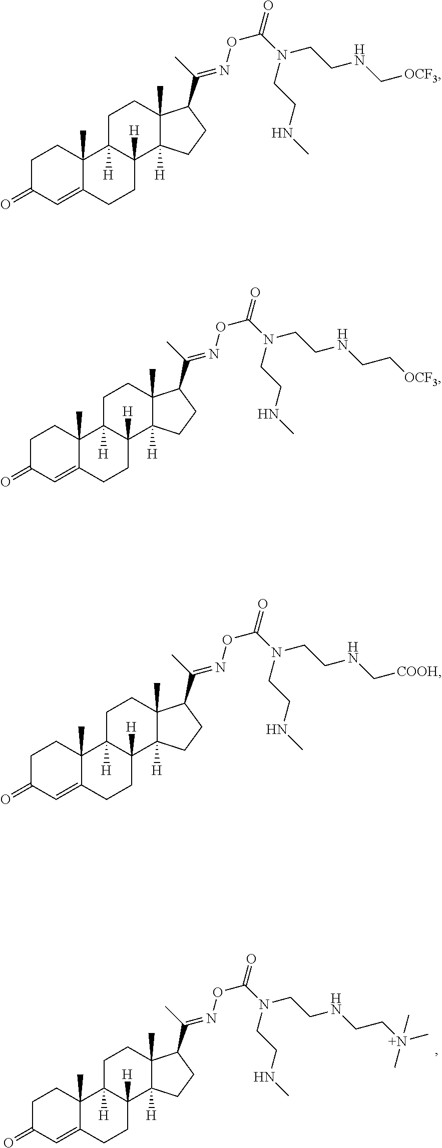

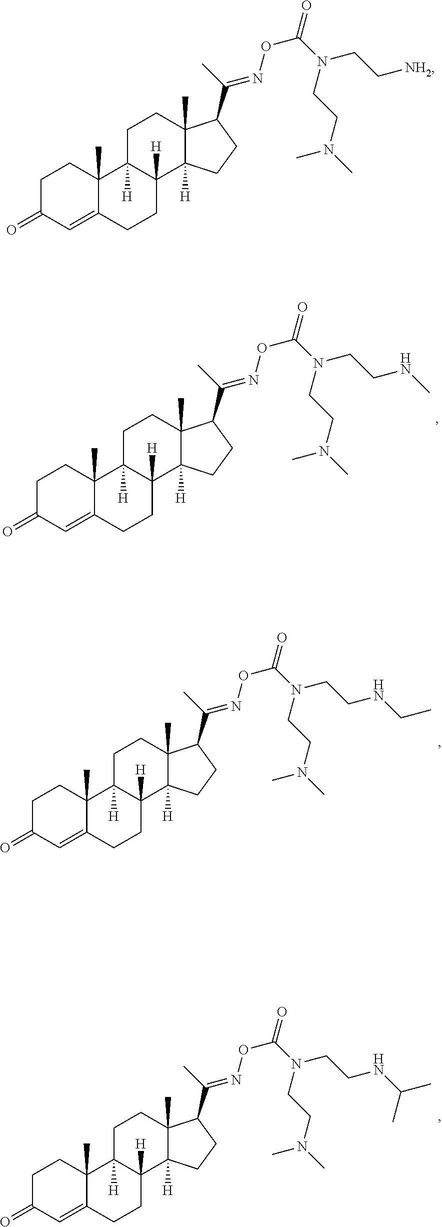

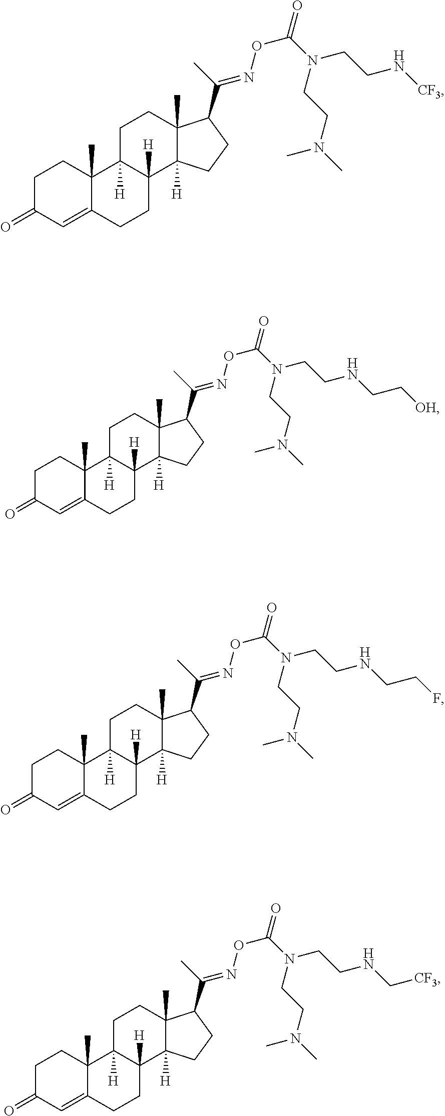

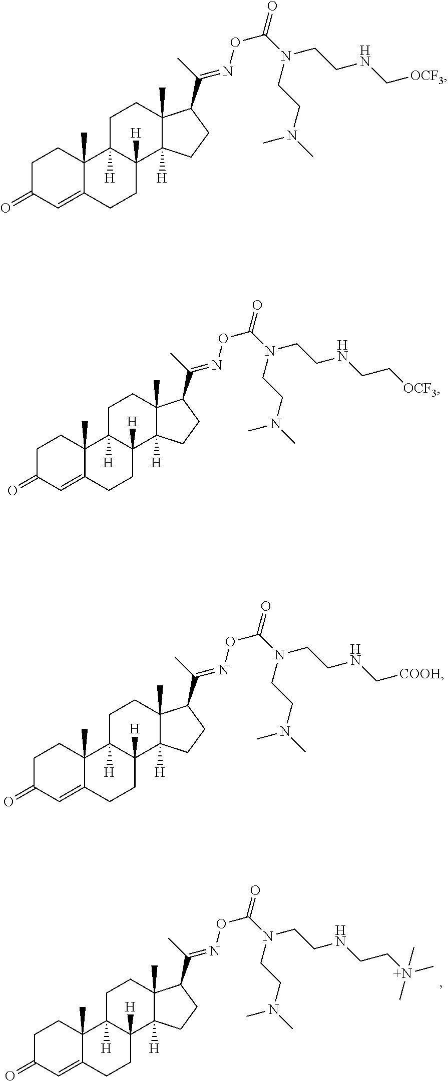

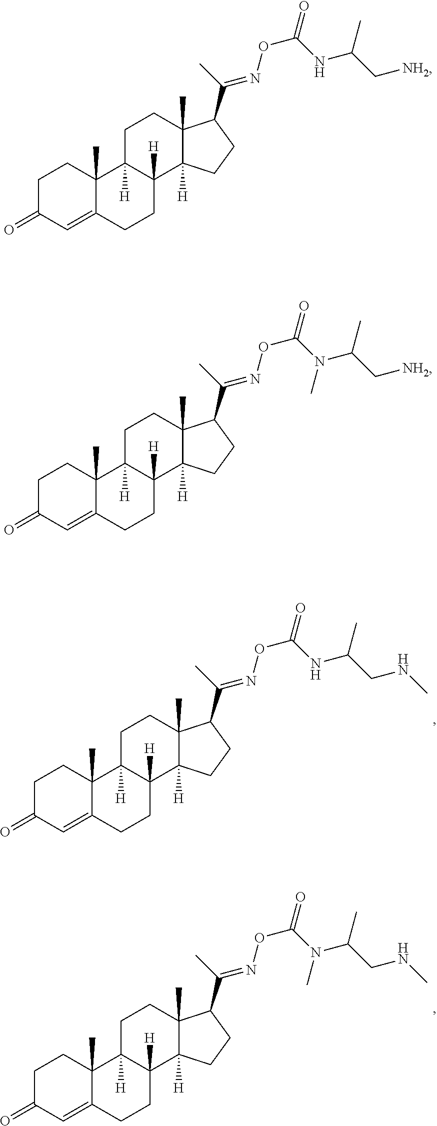

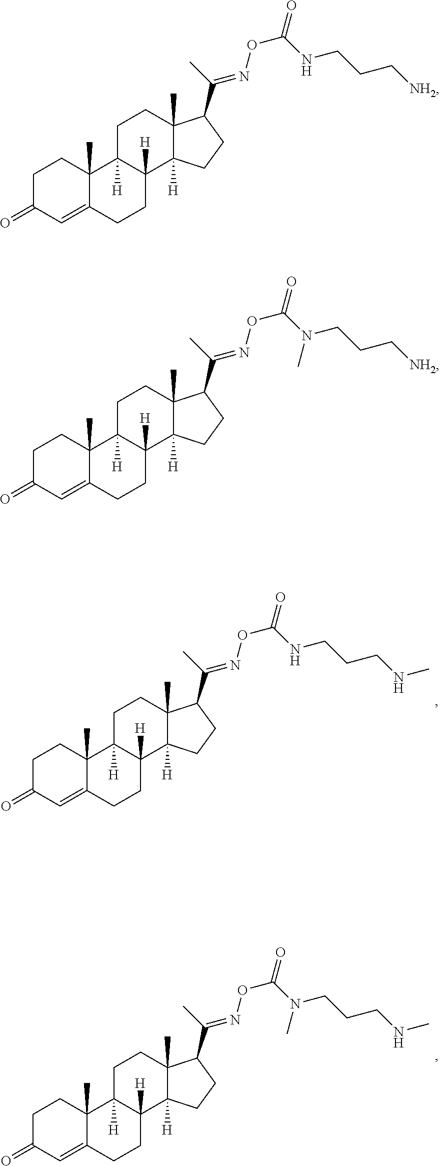

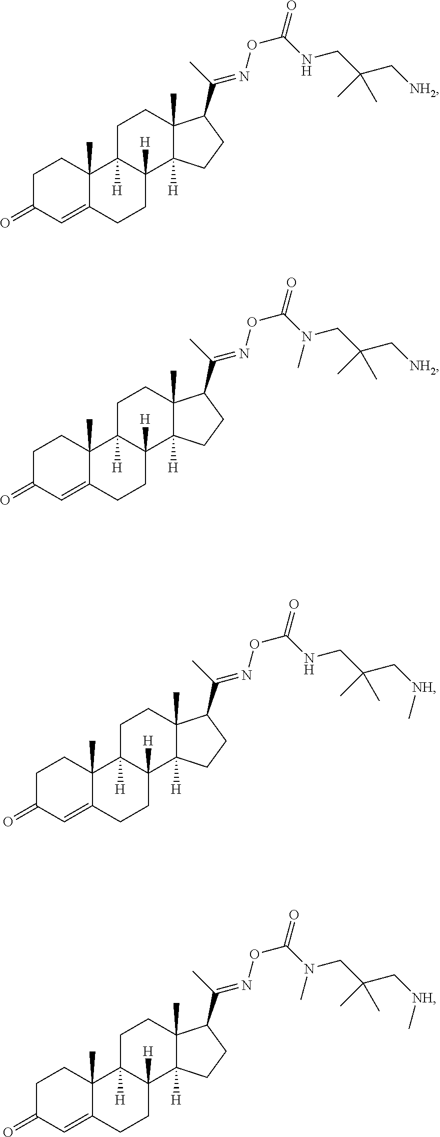

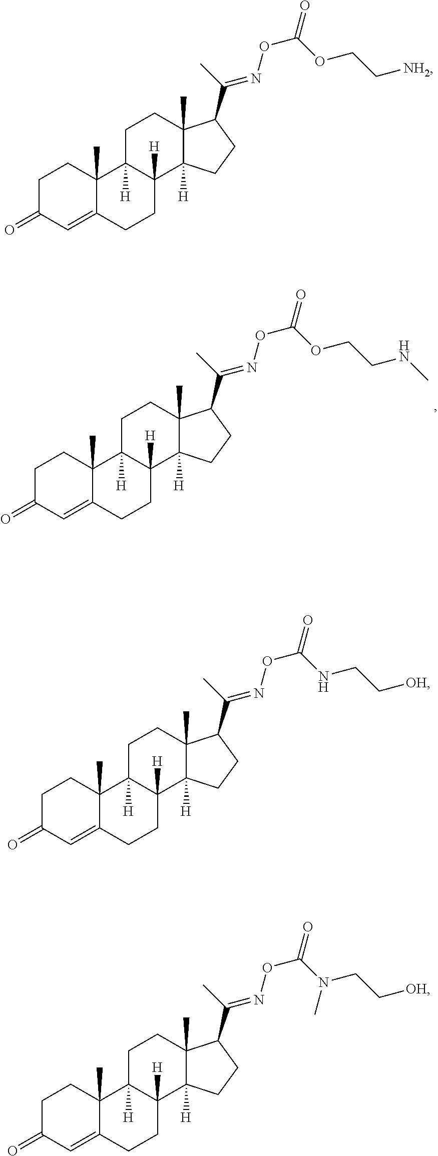

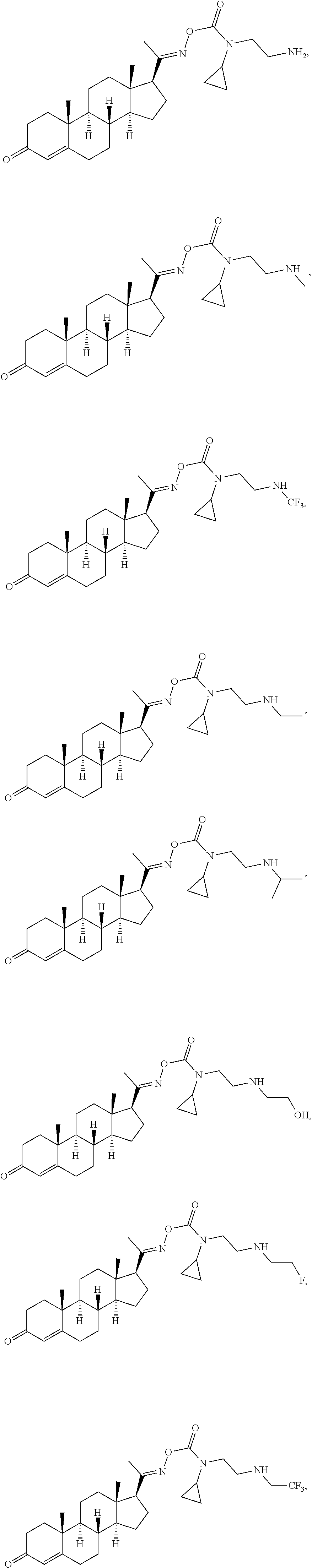

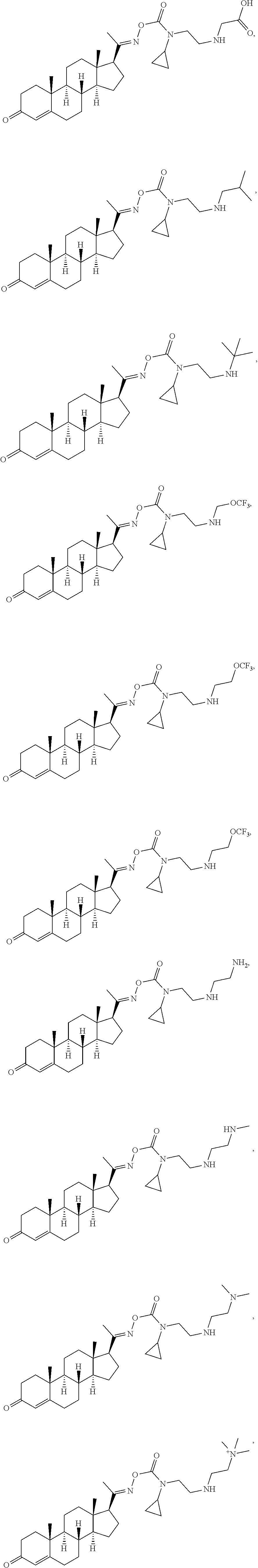

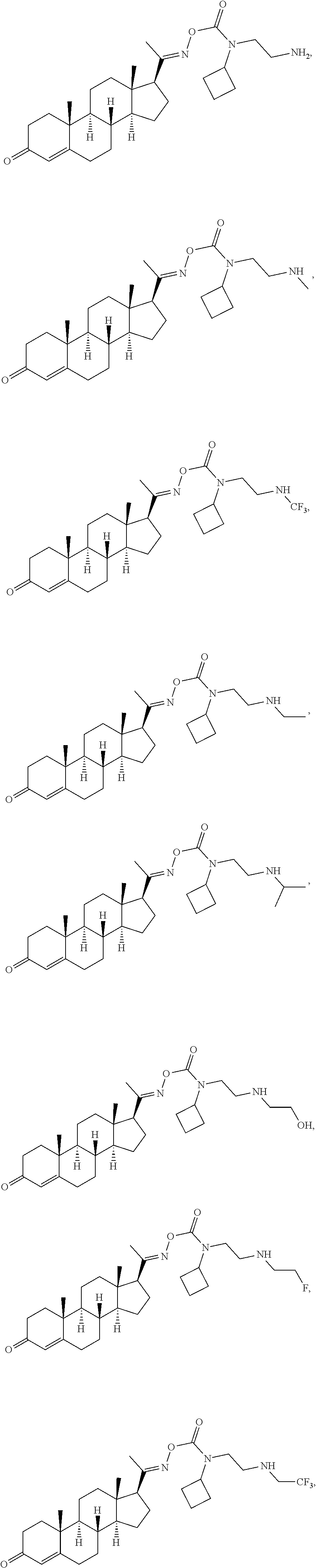

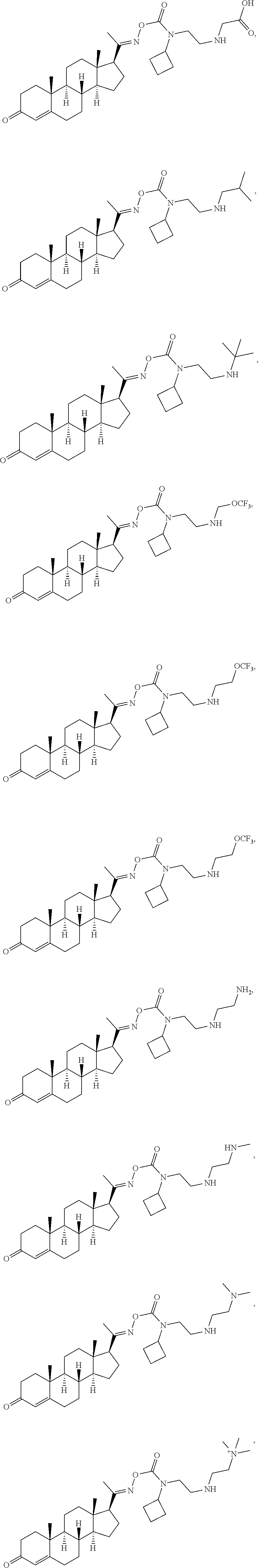

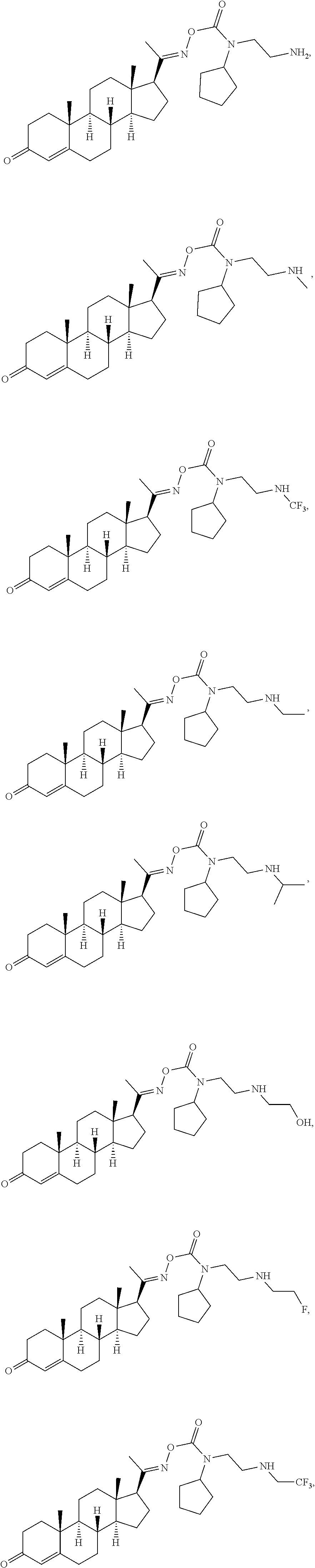

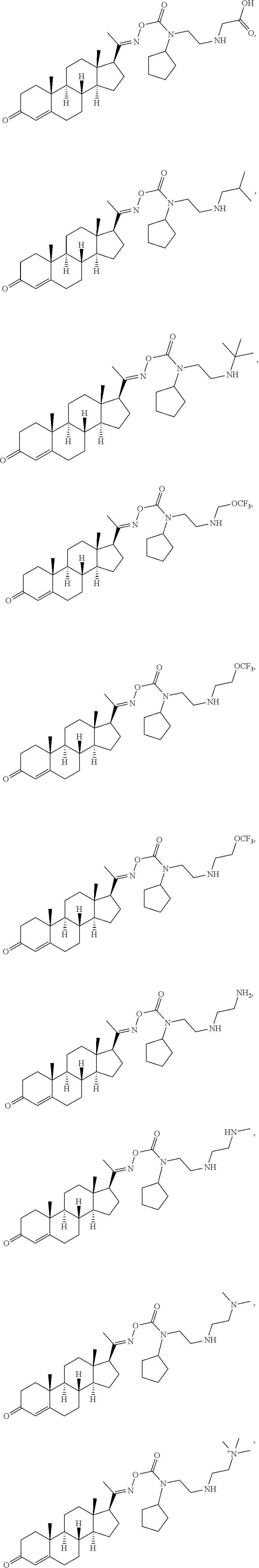

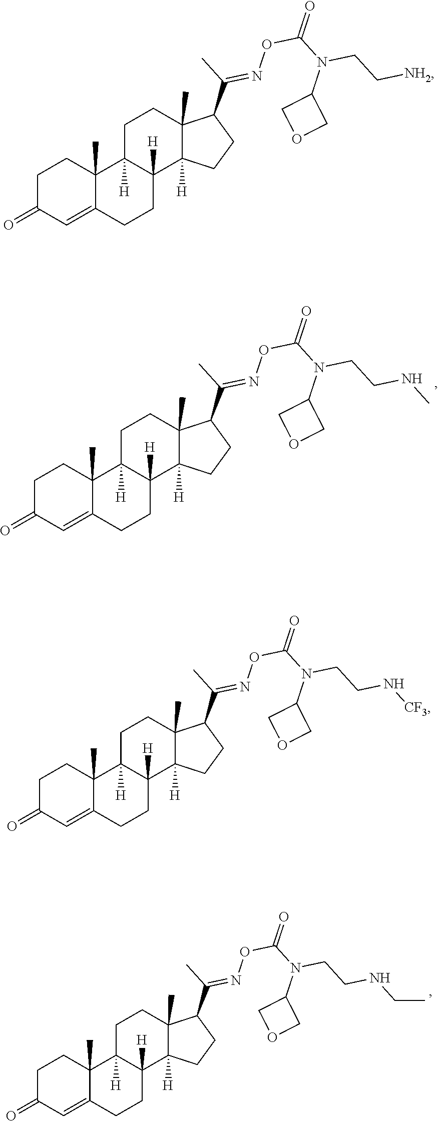

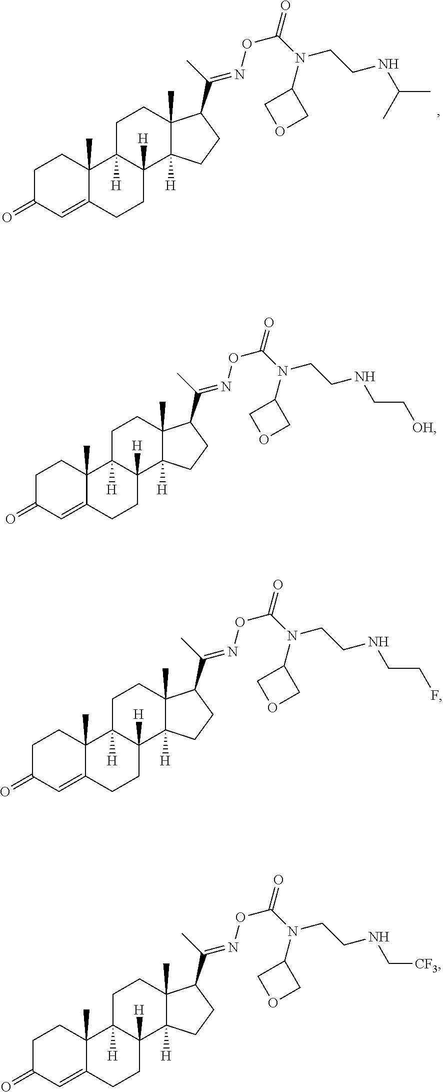

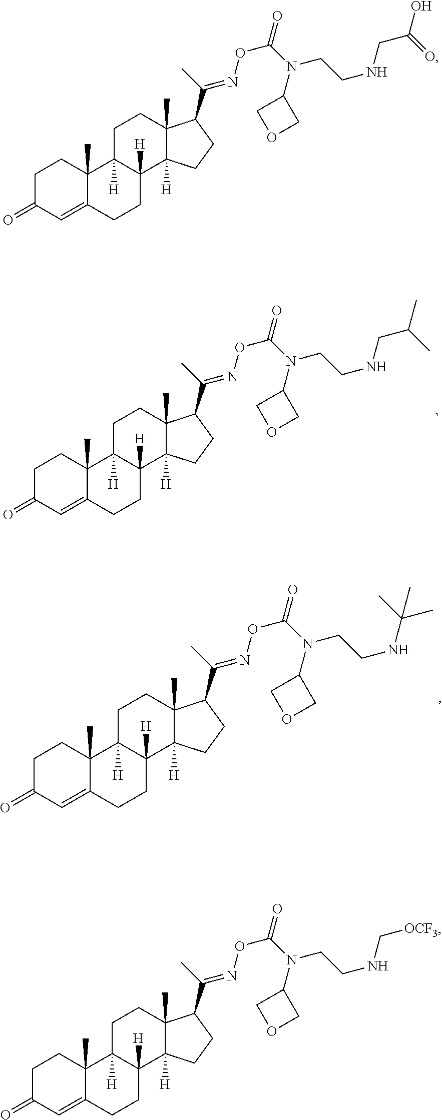

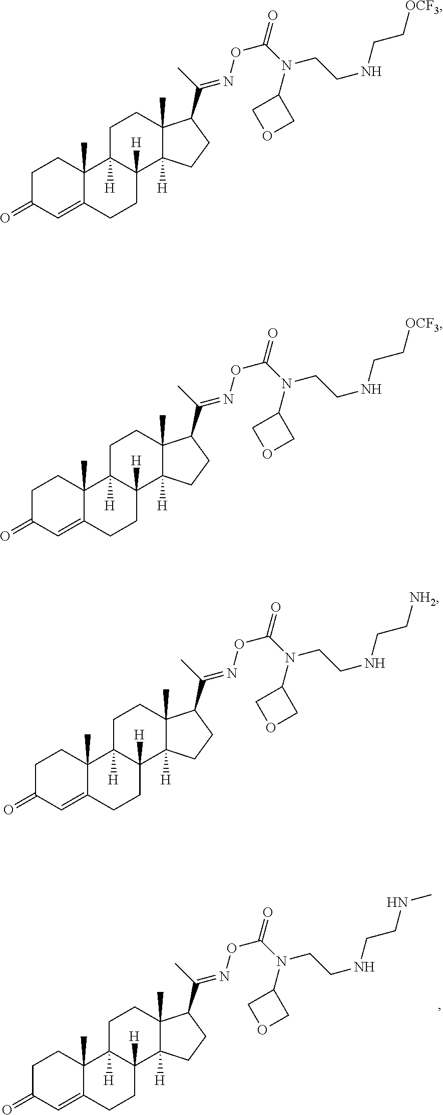

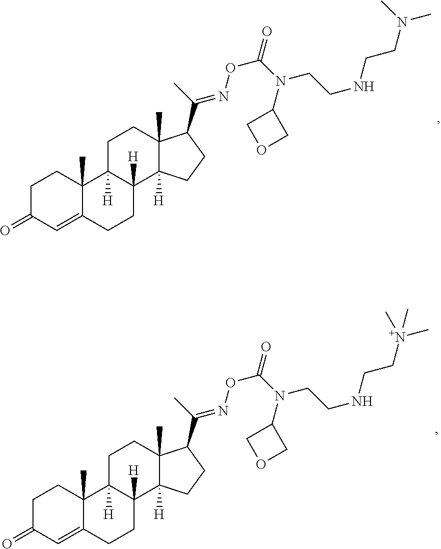

and pharmaceutically acceptable salts thereof.

Exemplary compounds of Formula II-1 also include:

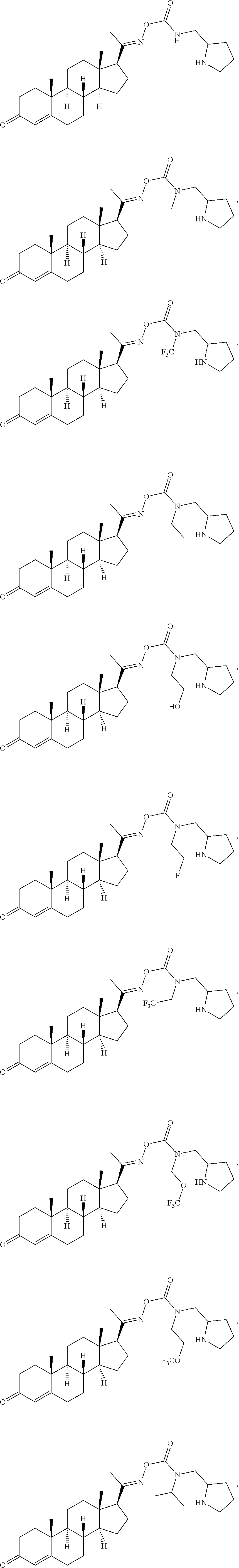

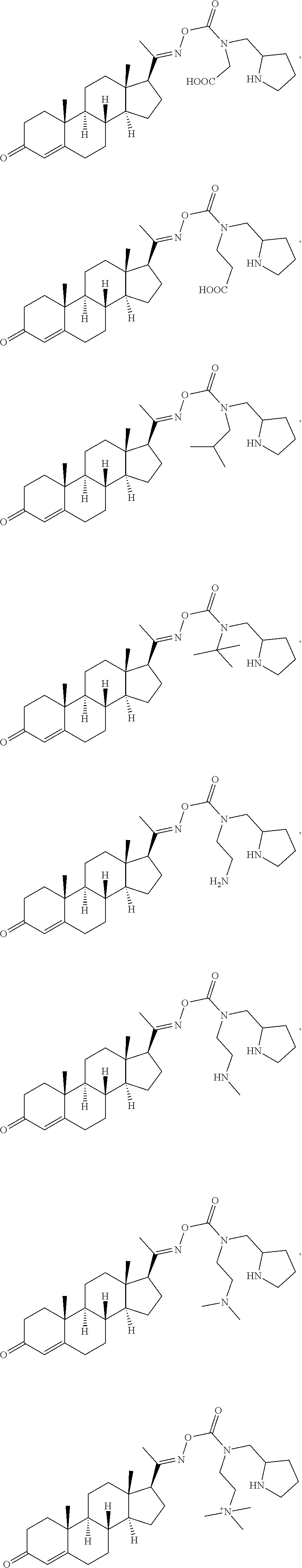

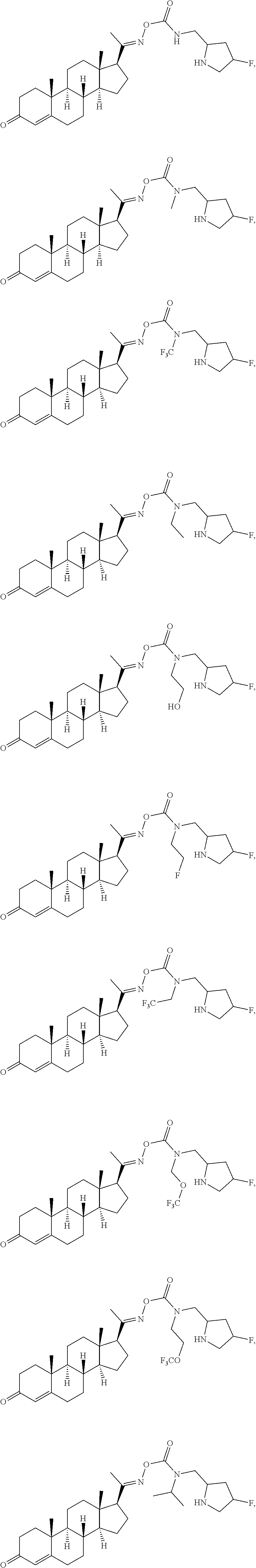

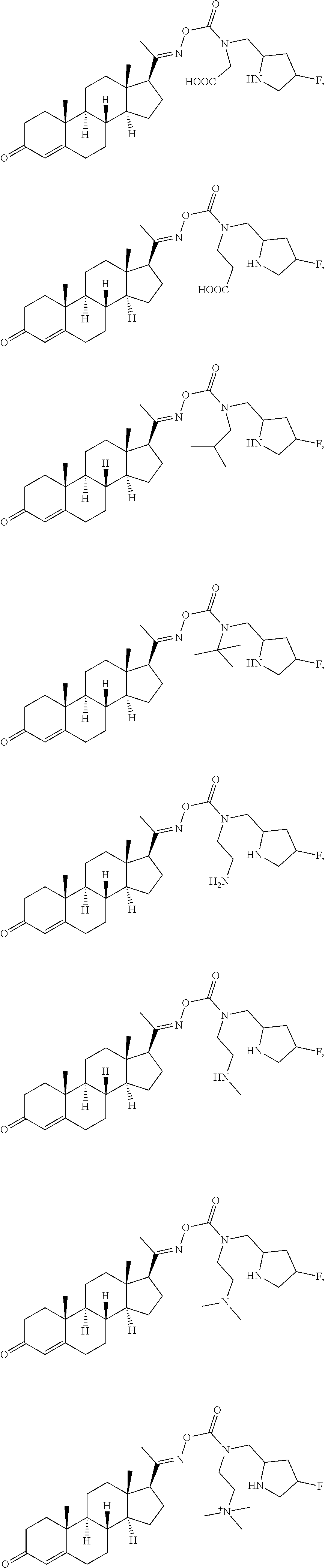

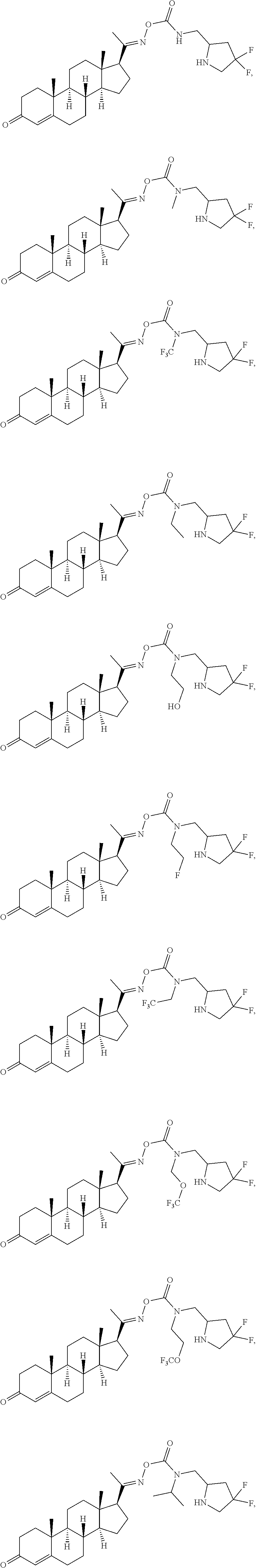

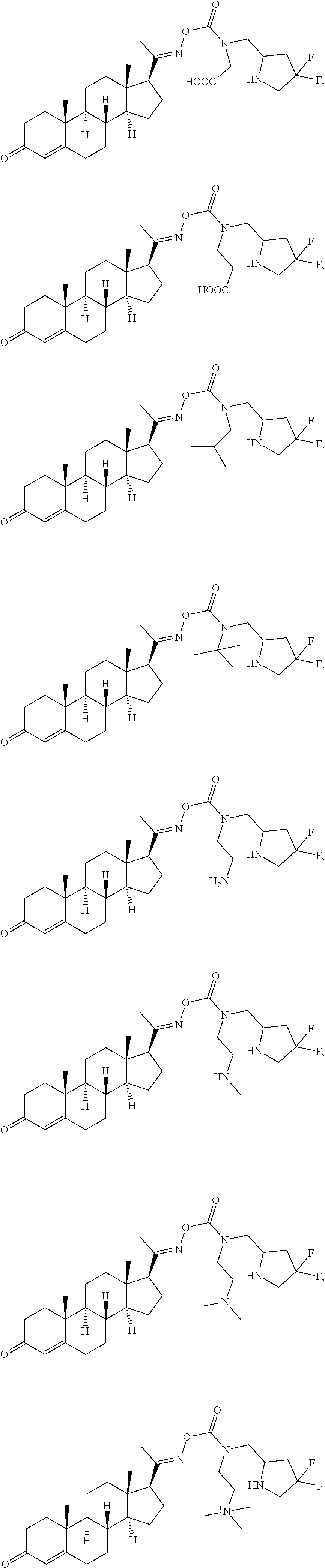

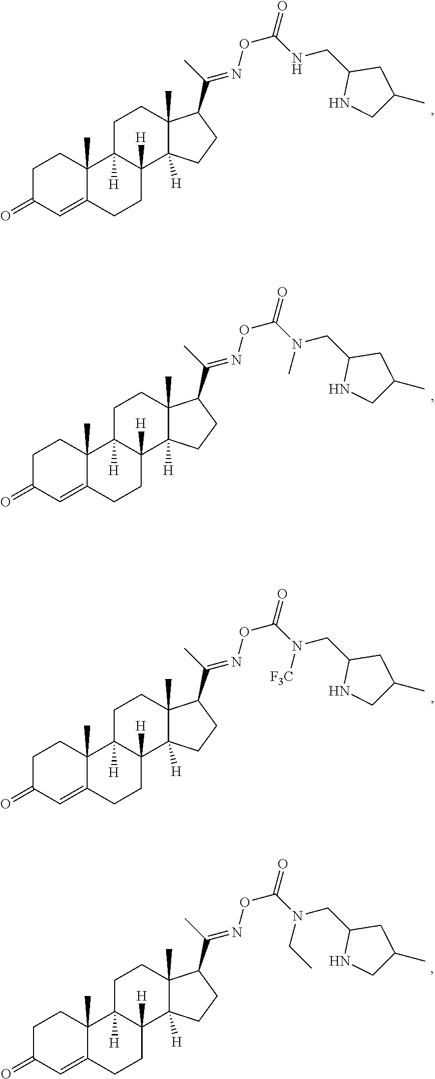

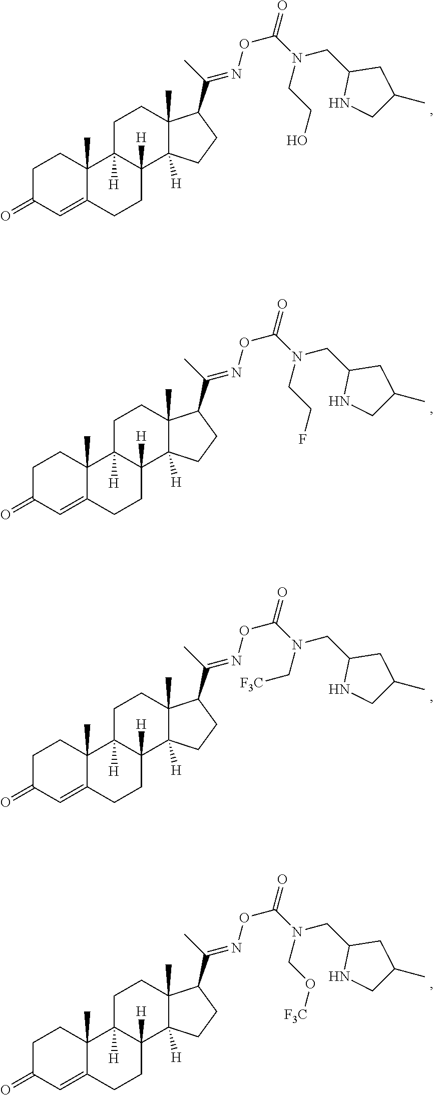

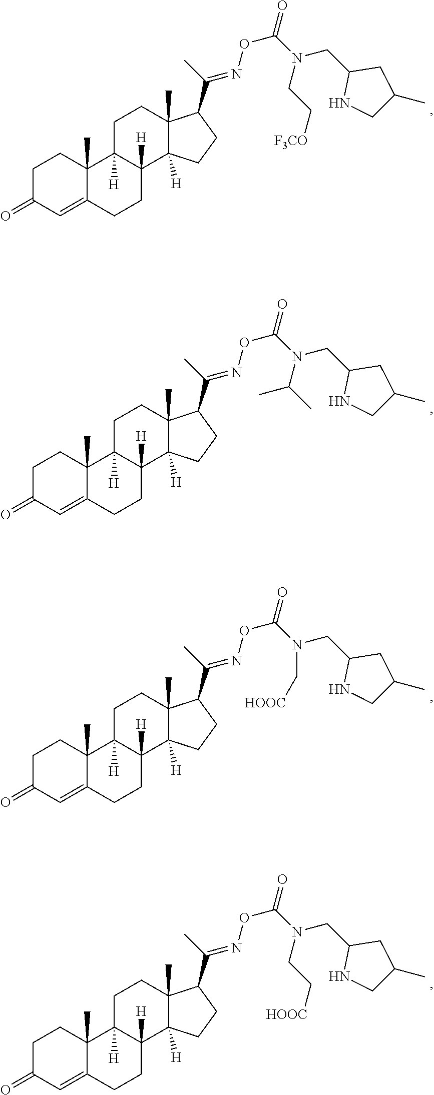

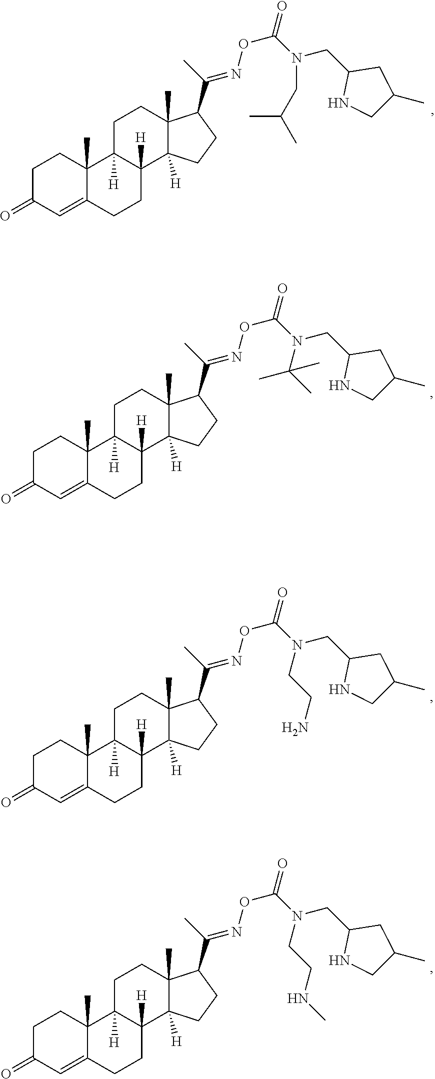

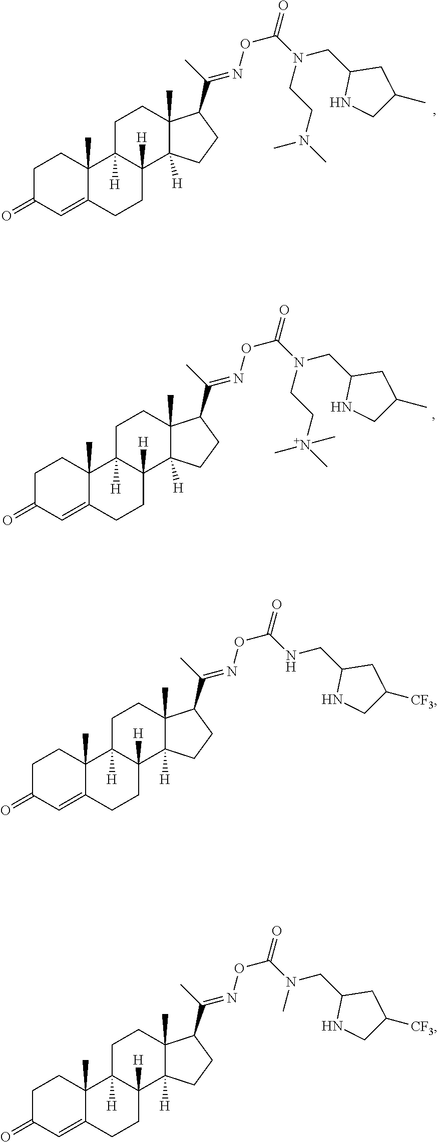

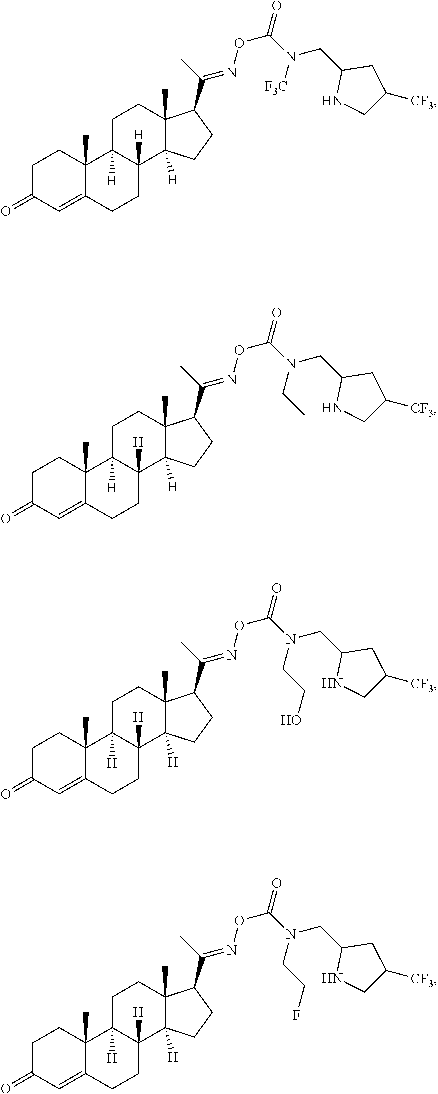

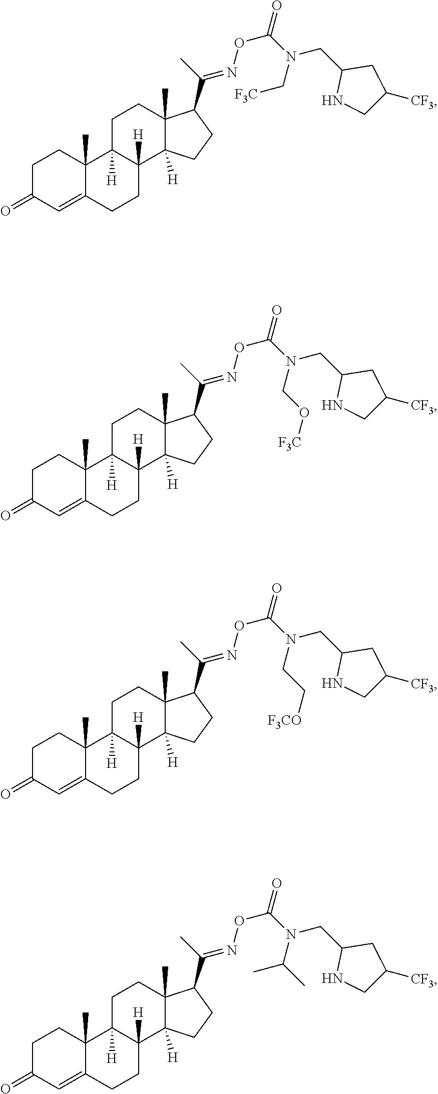

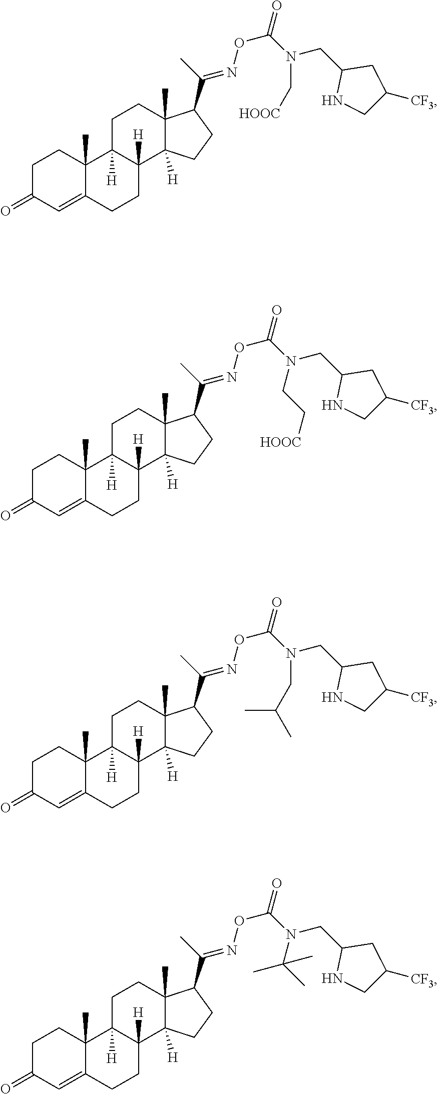

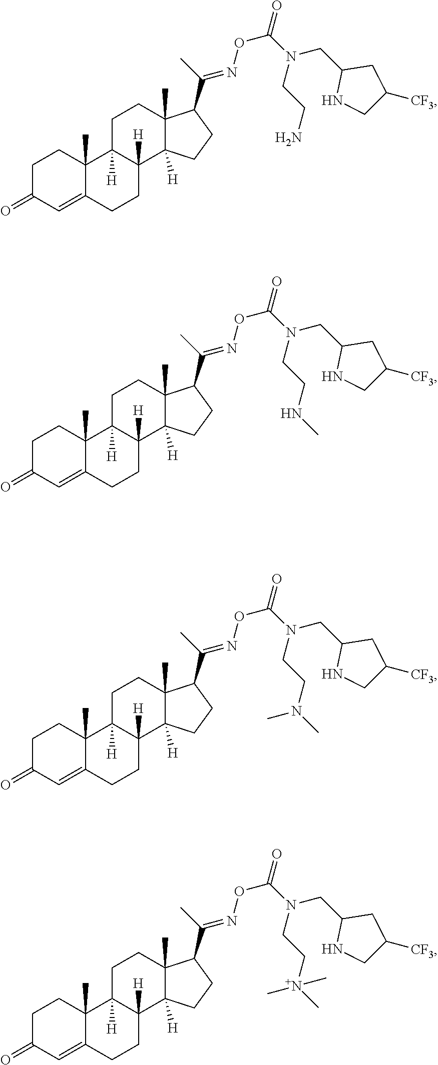

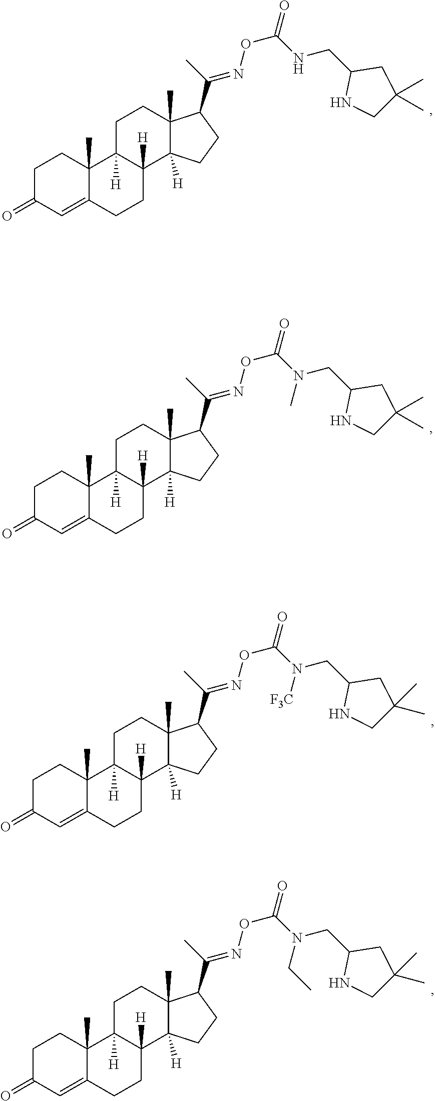

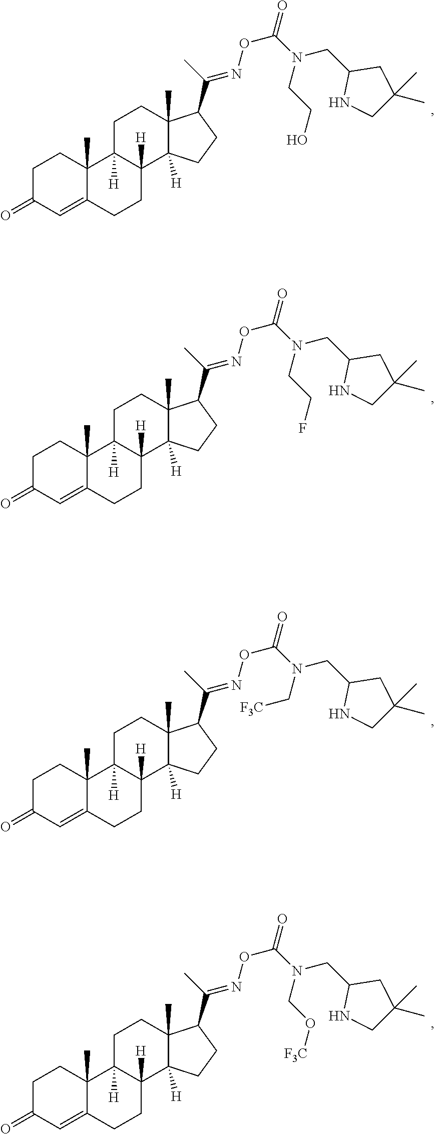

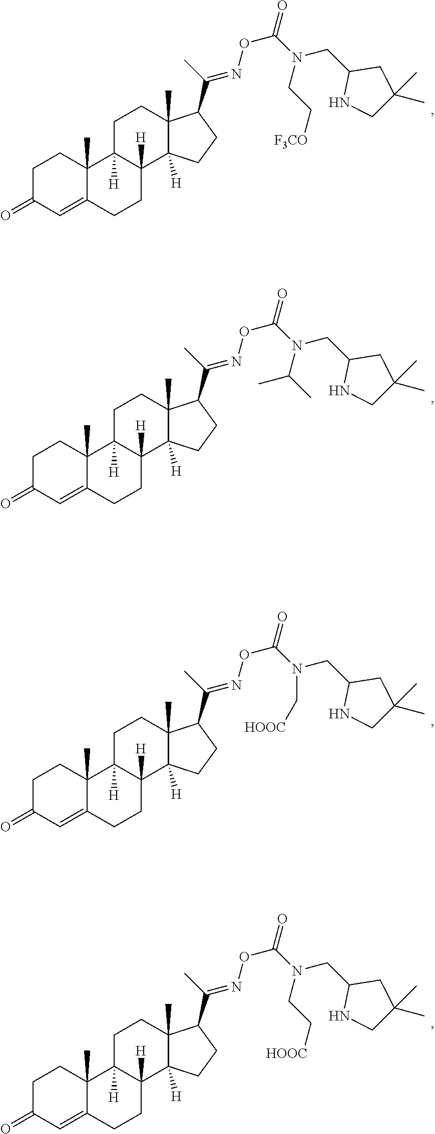

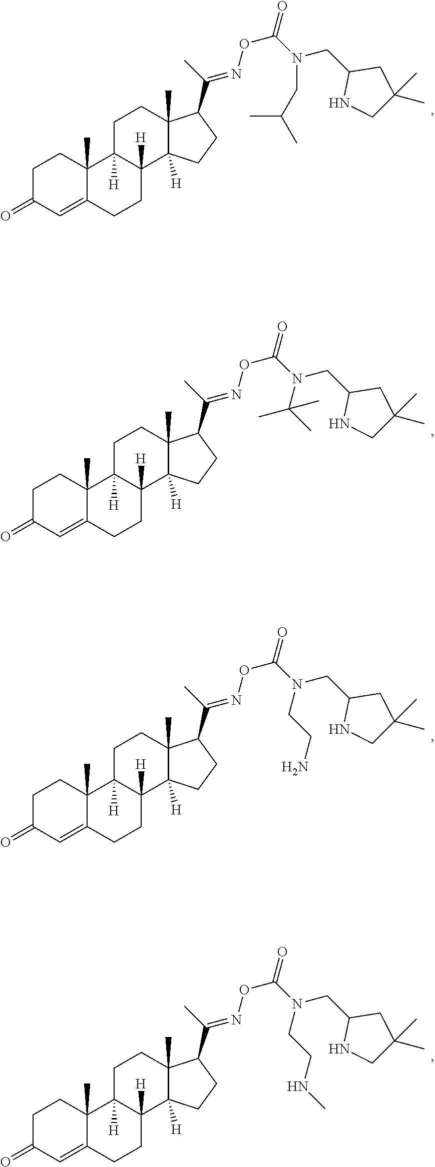

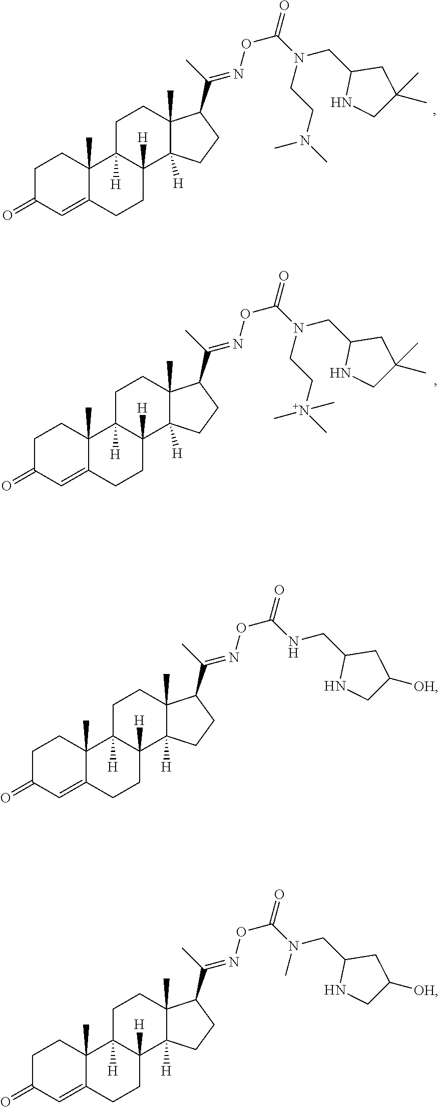

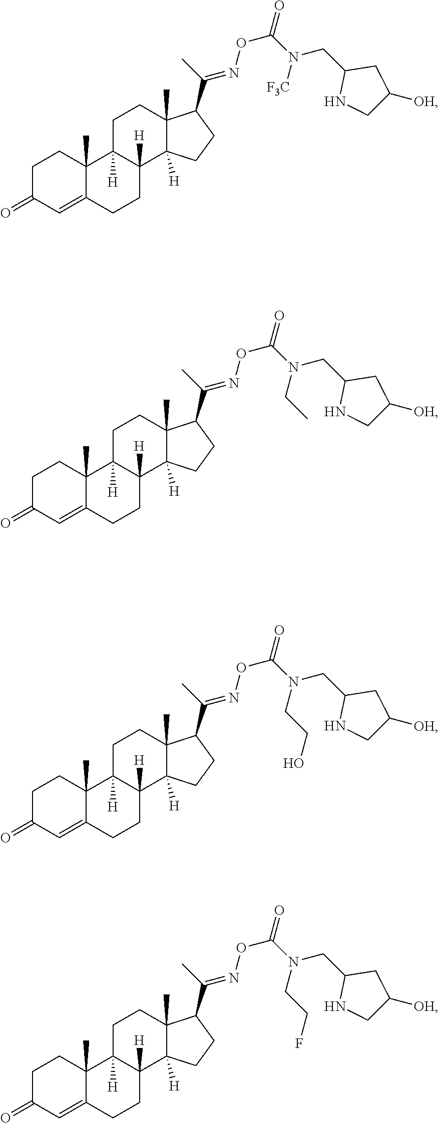

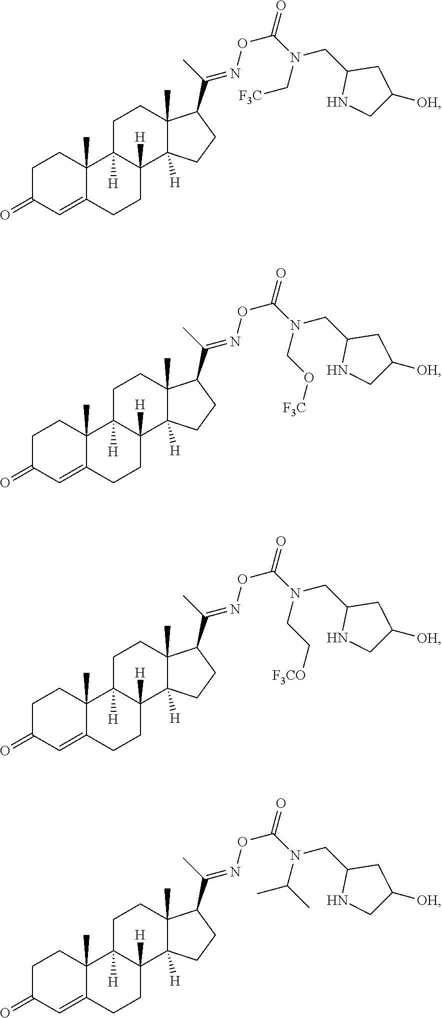

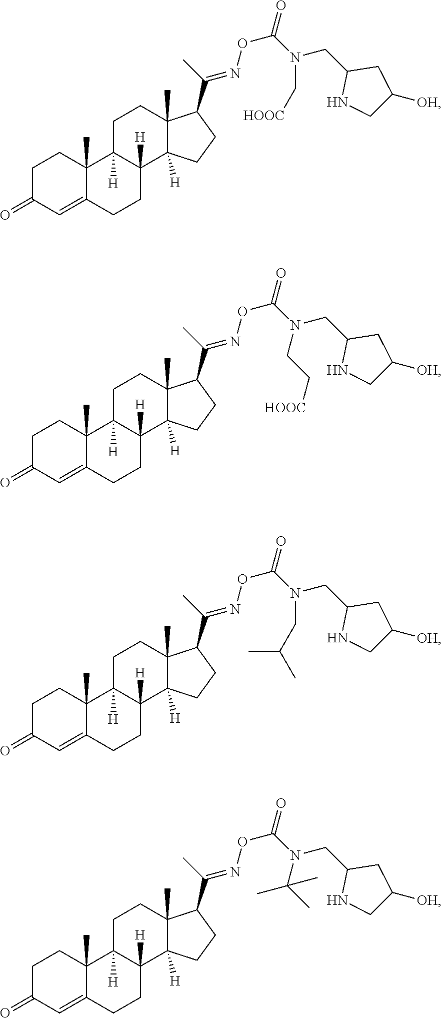

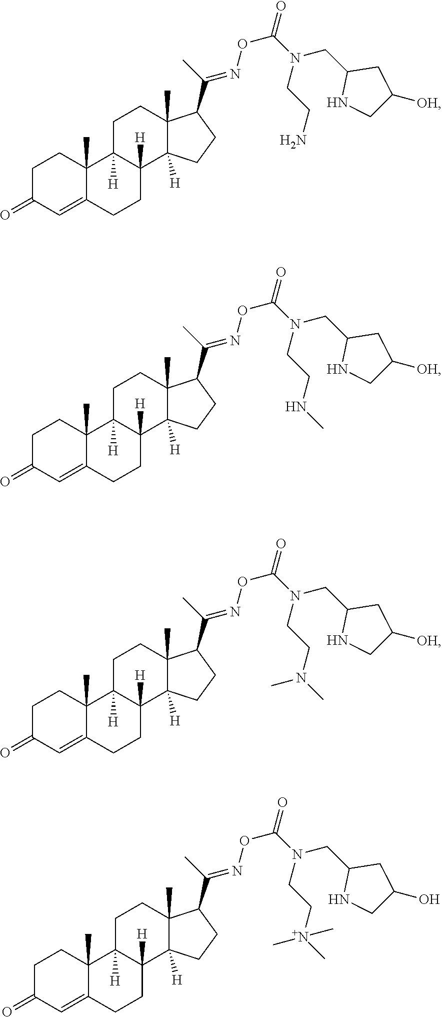

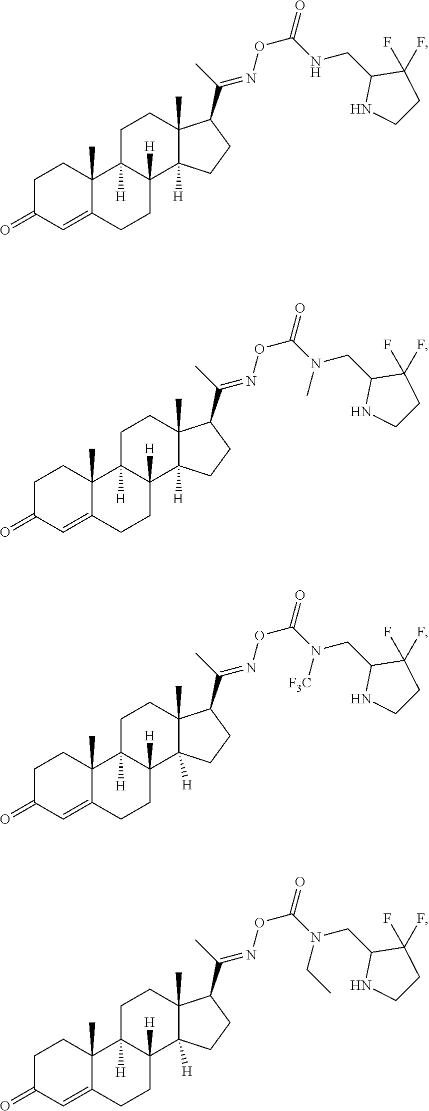

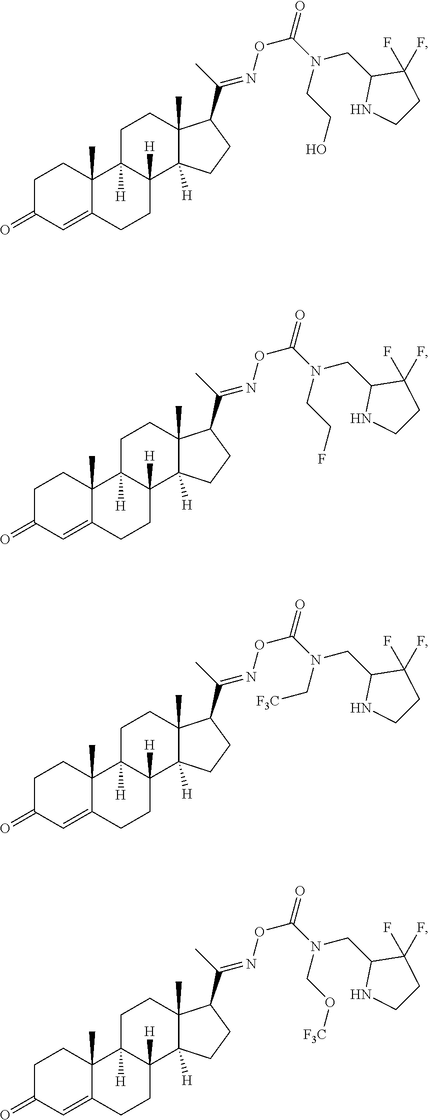

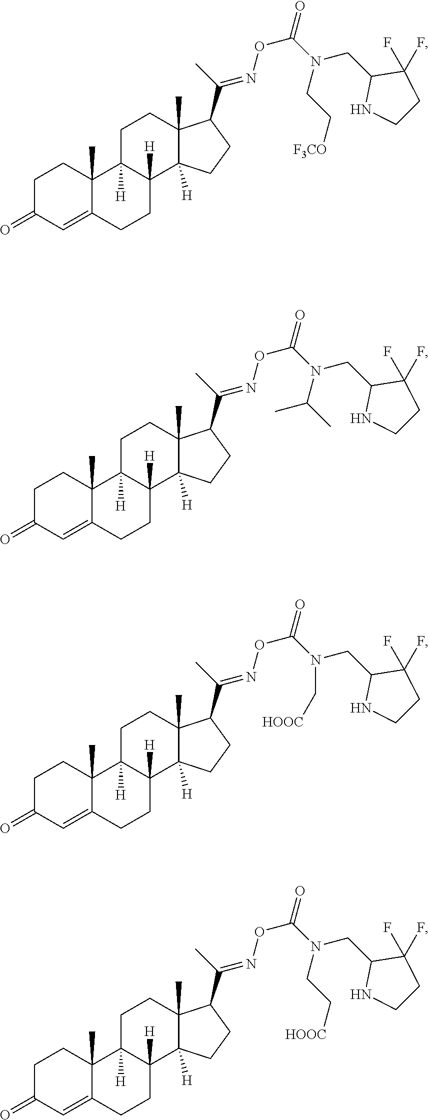

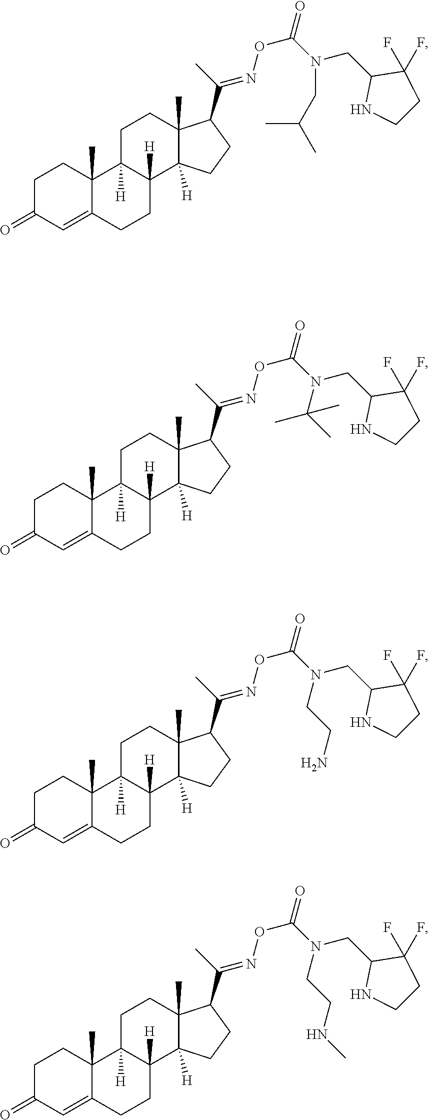

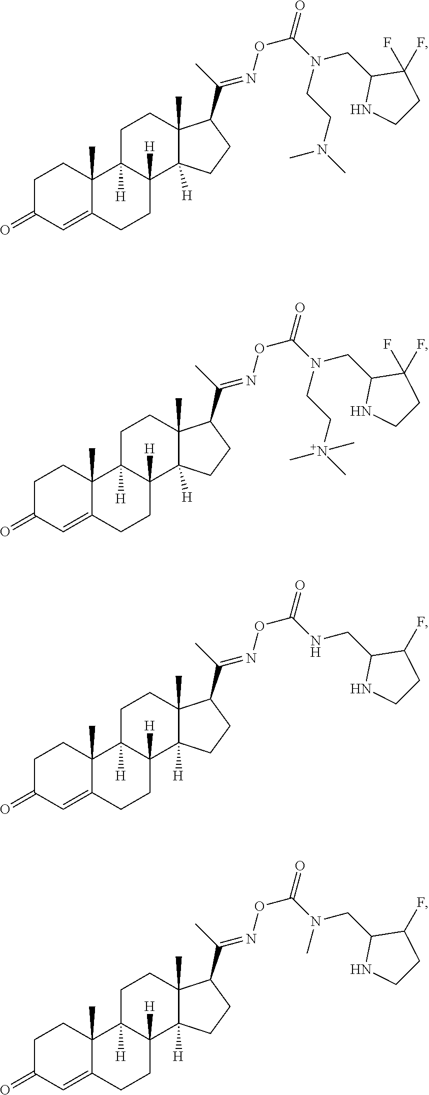

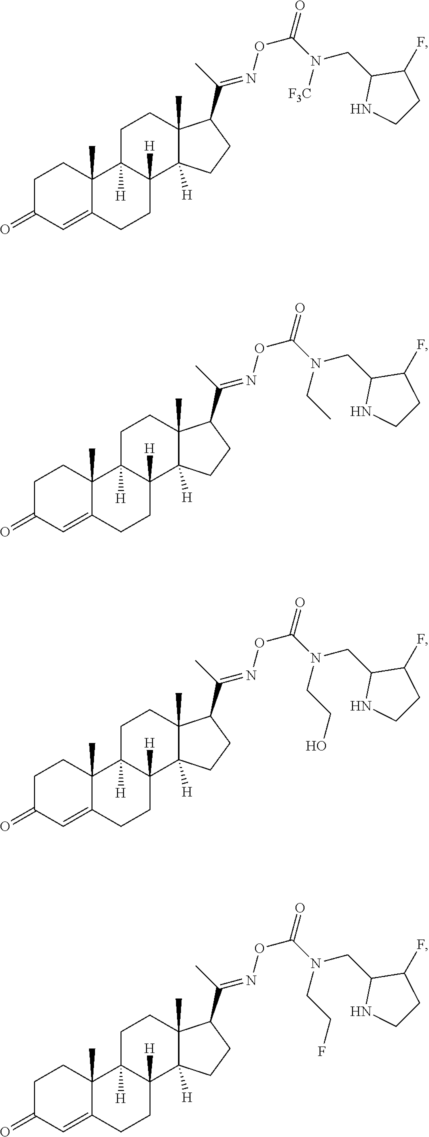

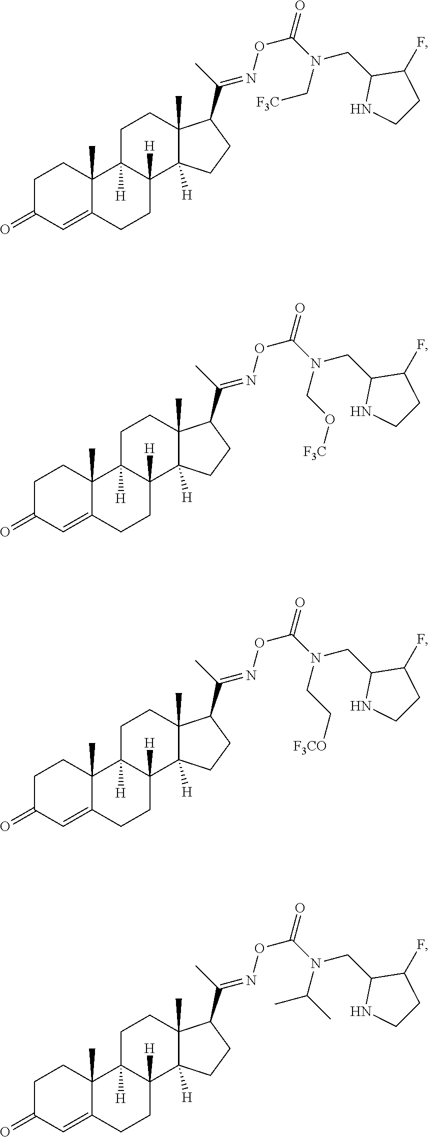

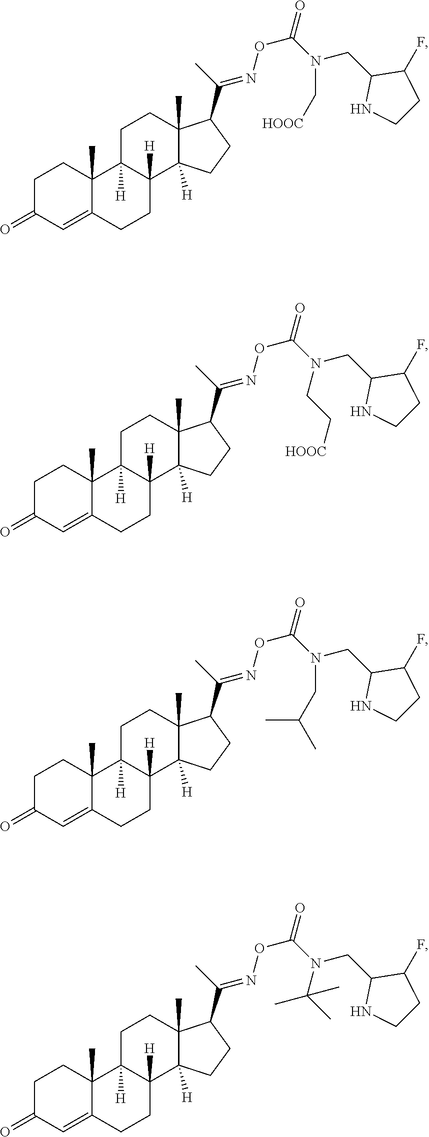

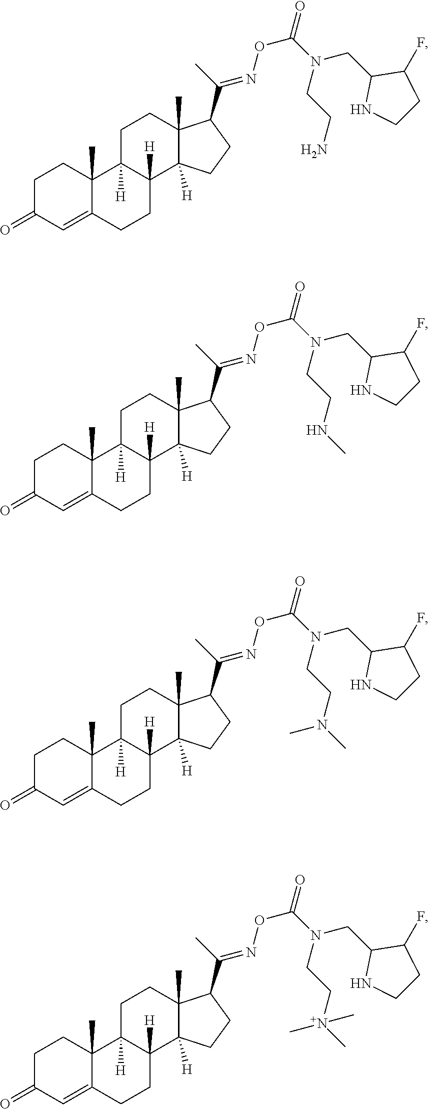

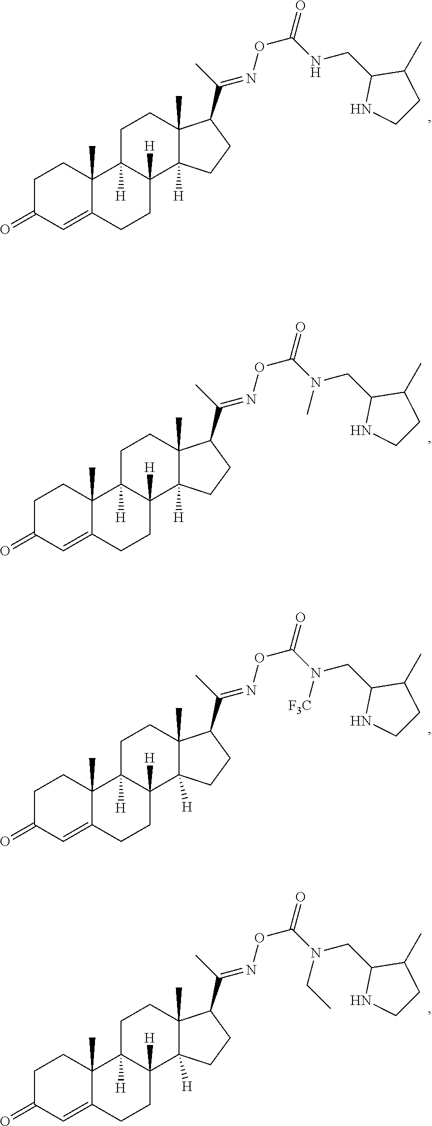

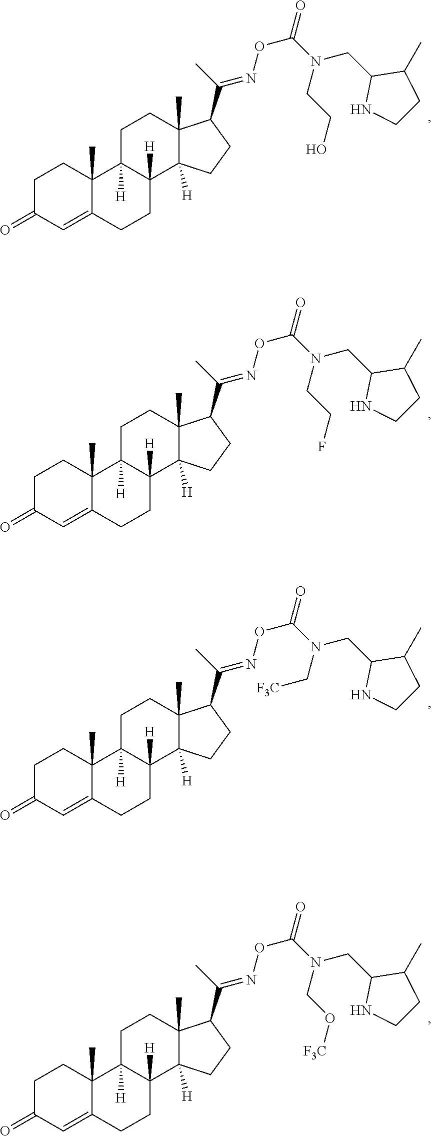

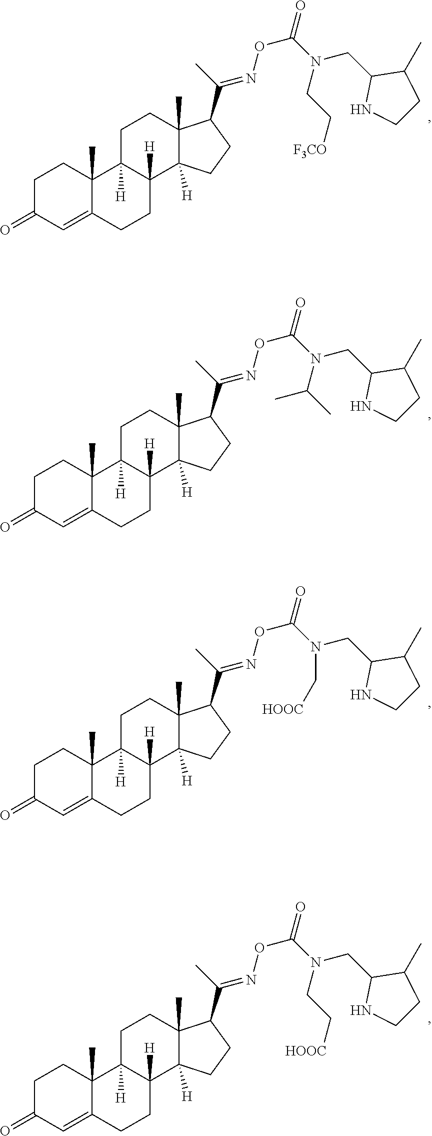

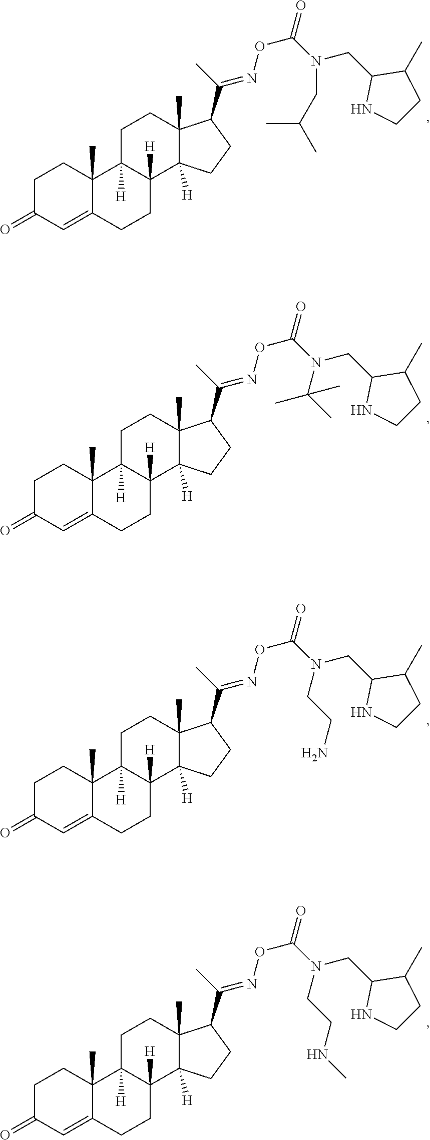

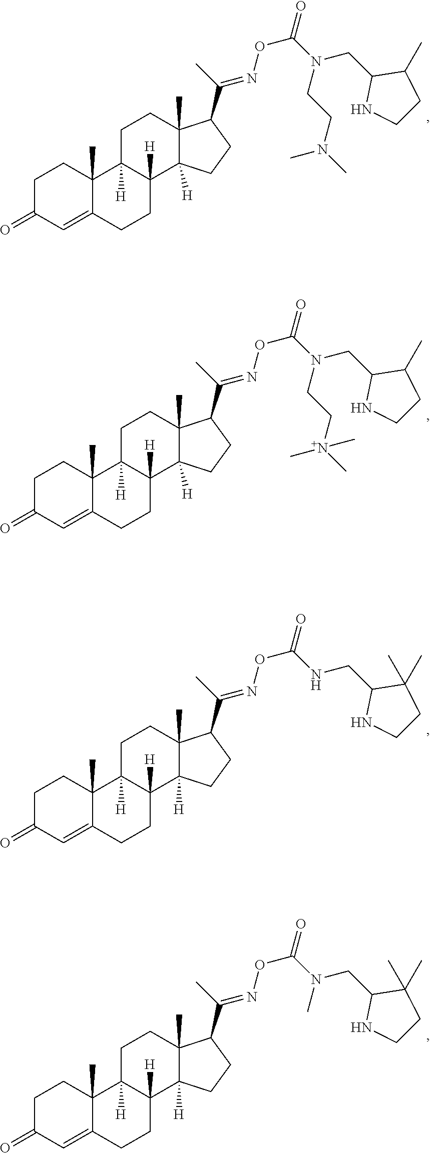

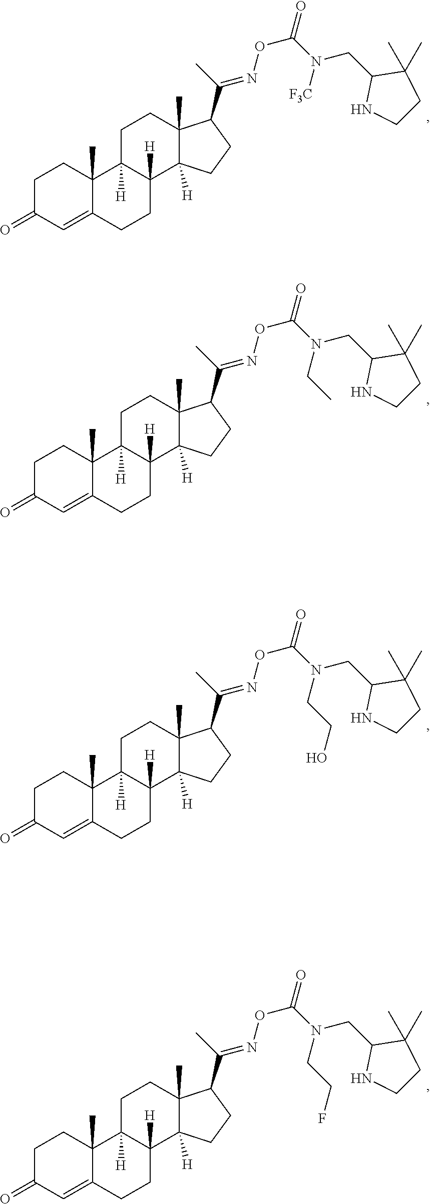

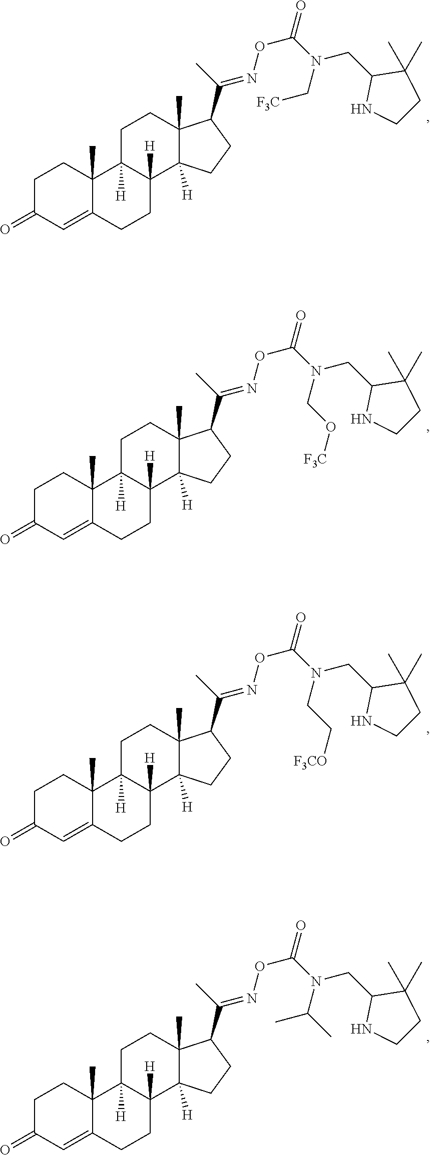

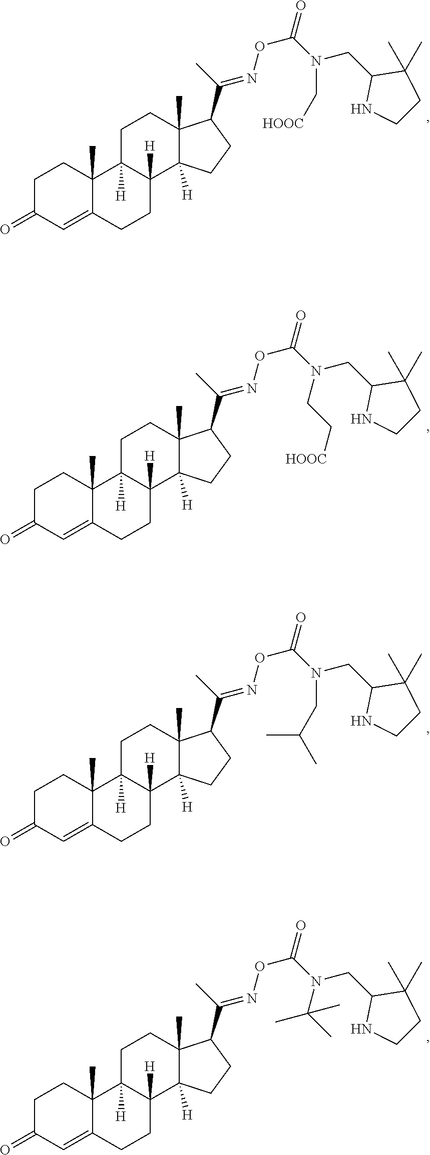

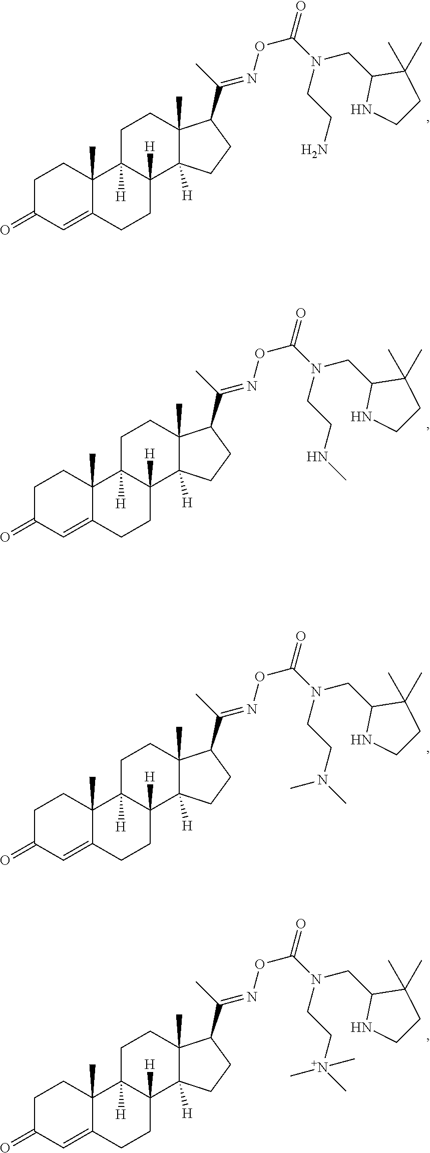

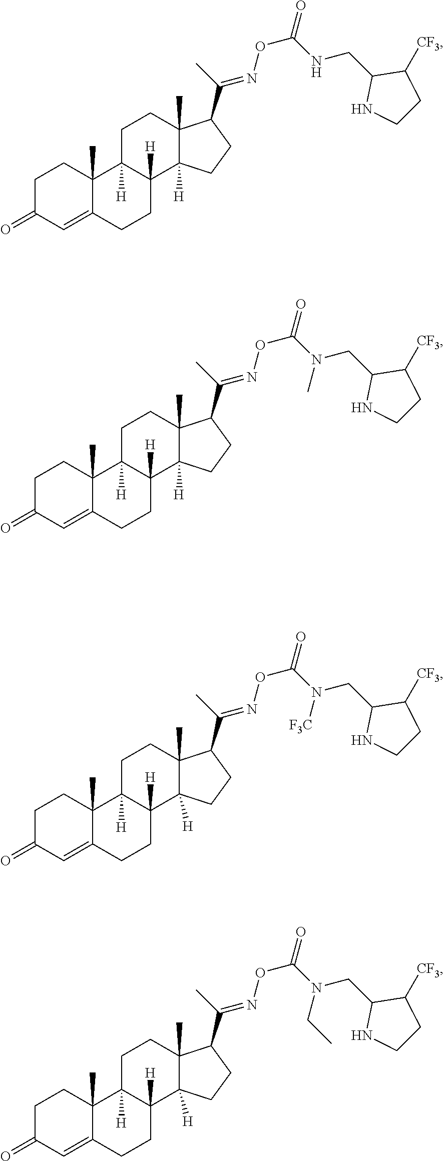

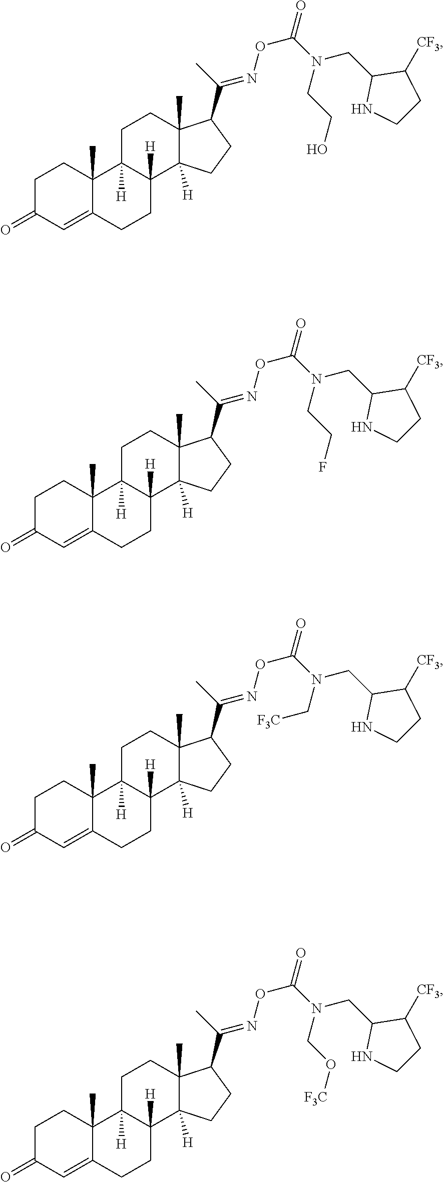

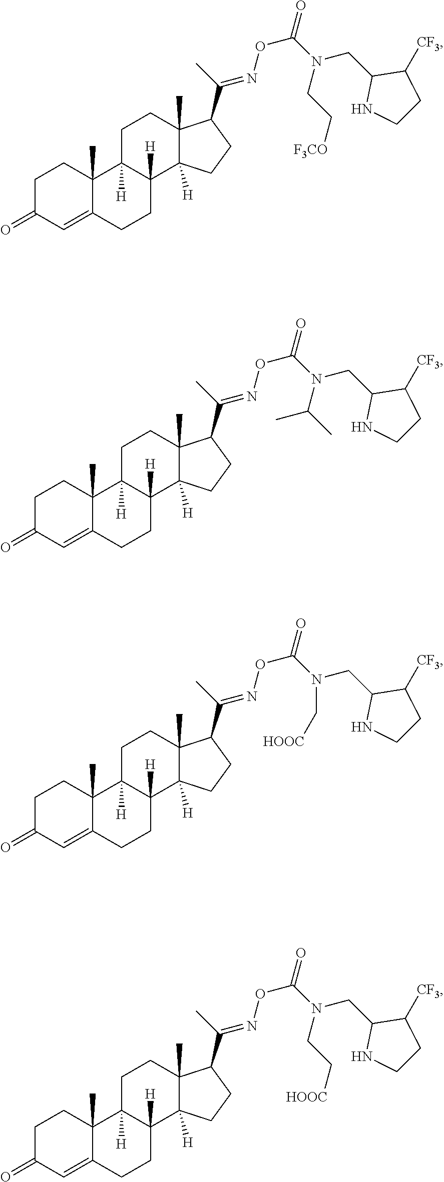

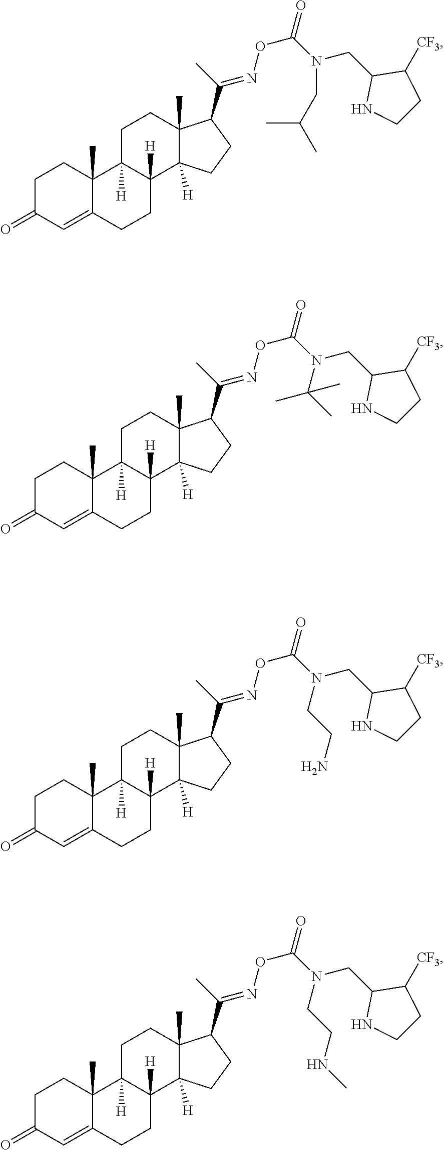

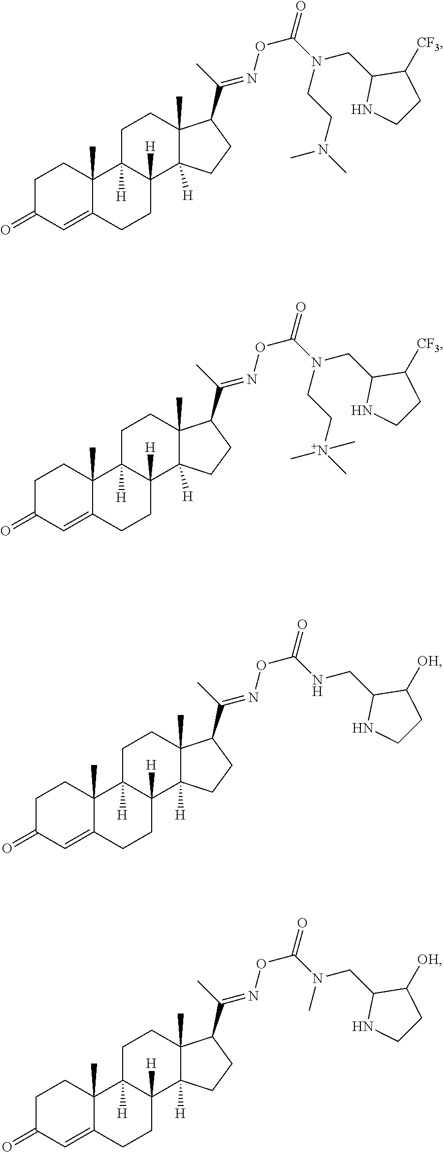

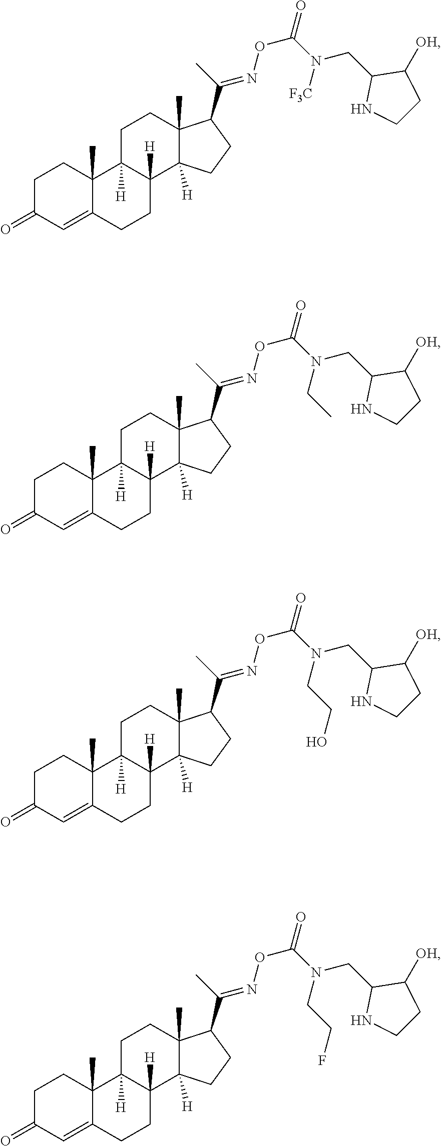

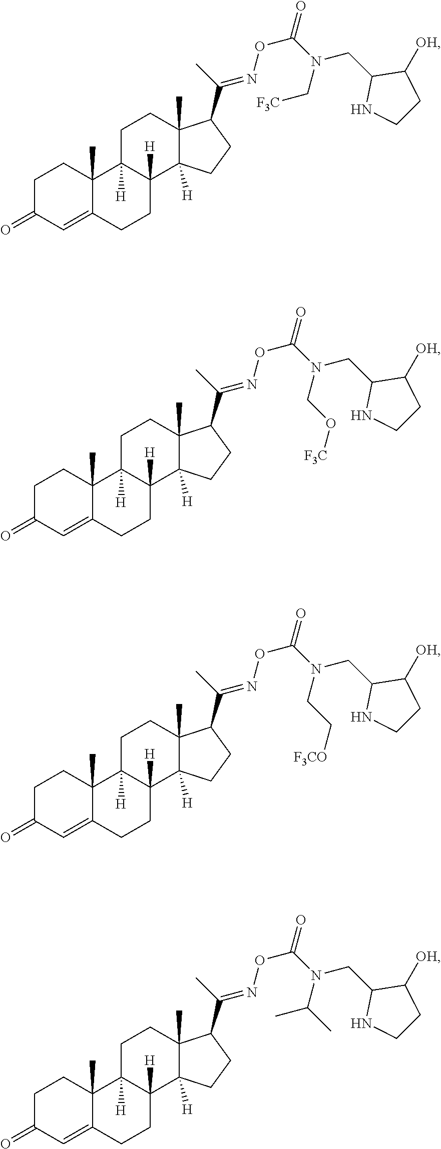

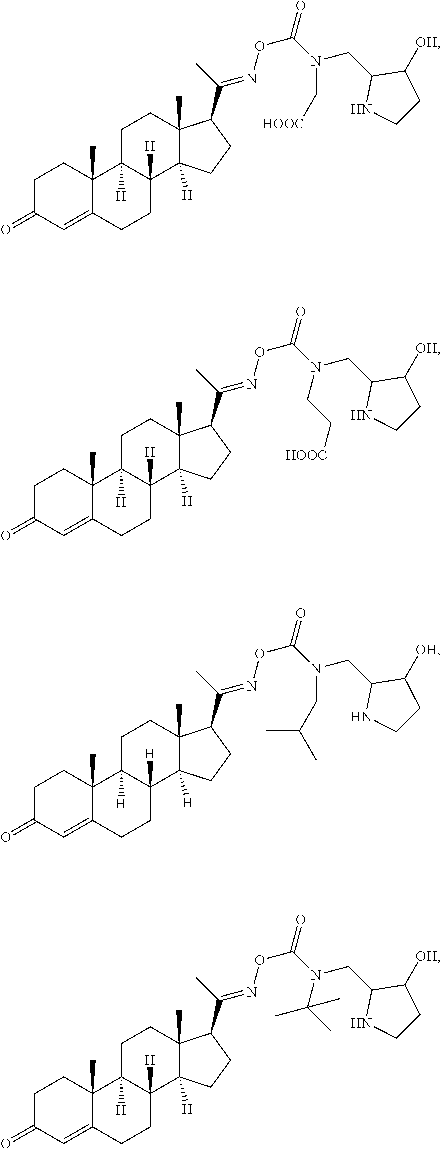

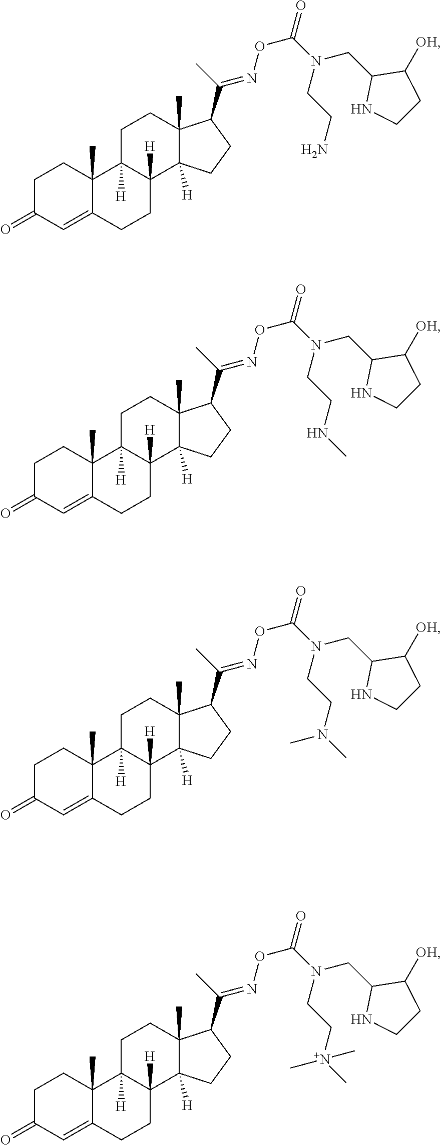

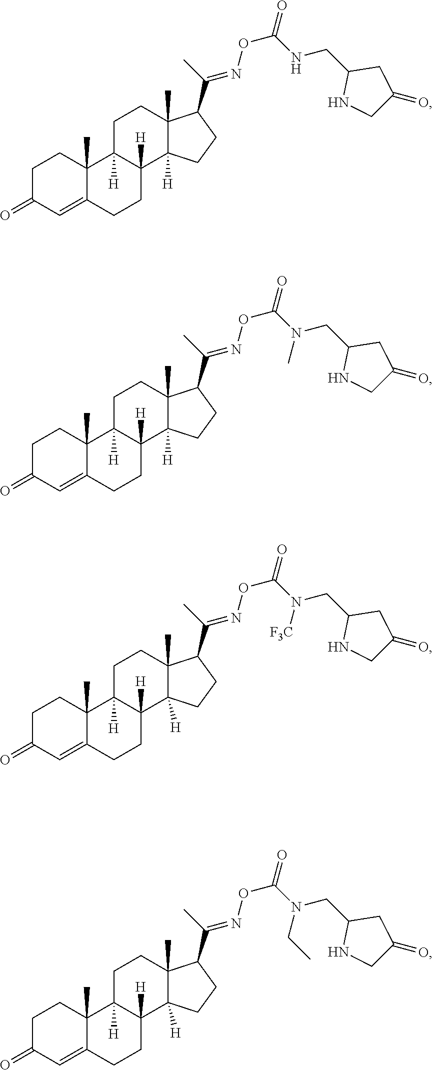

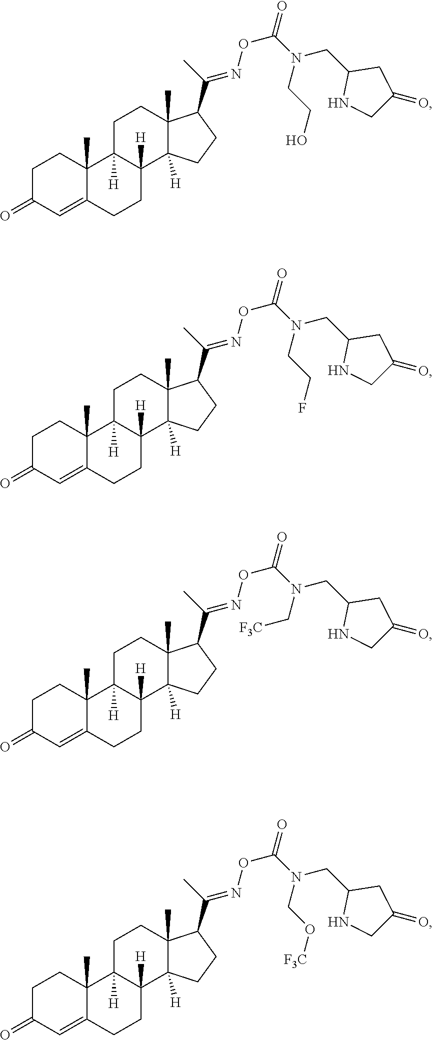

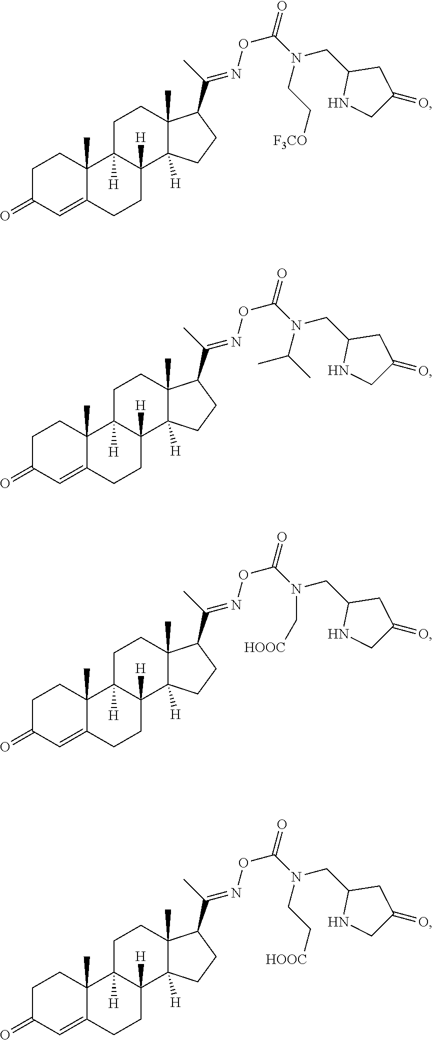

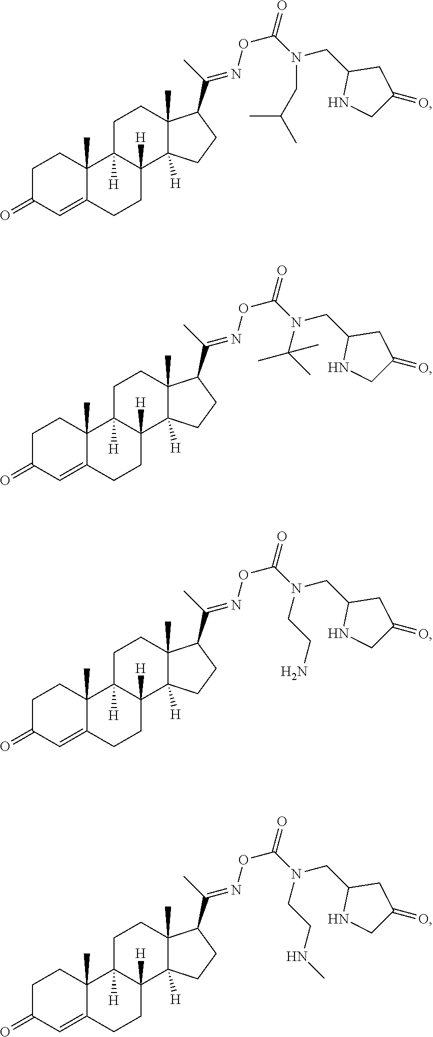

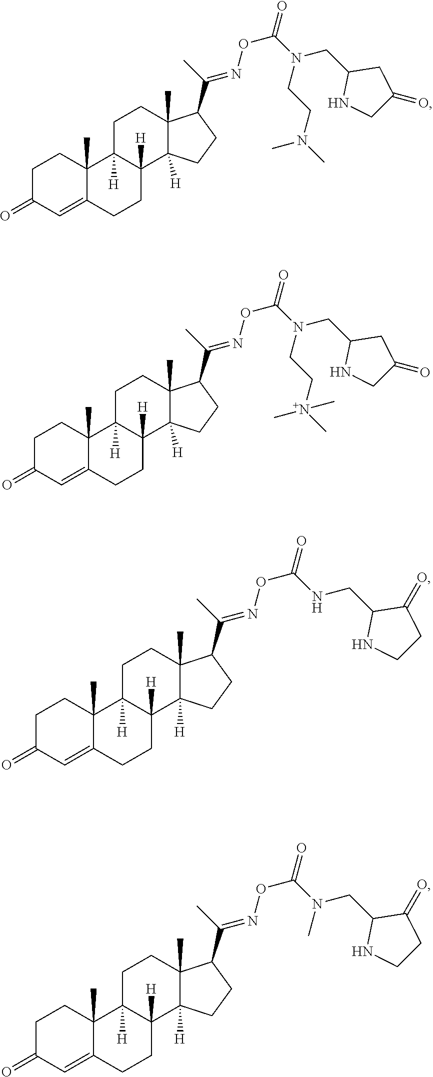

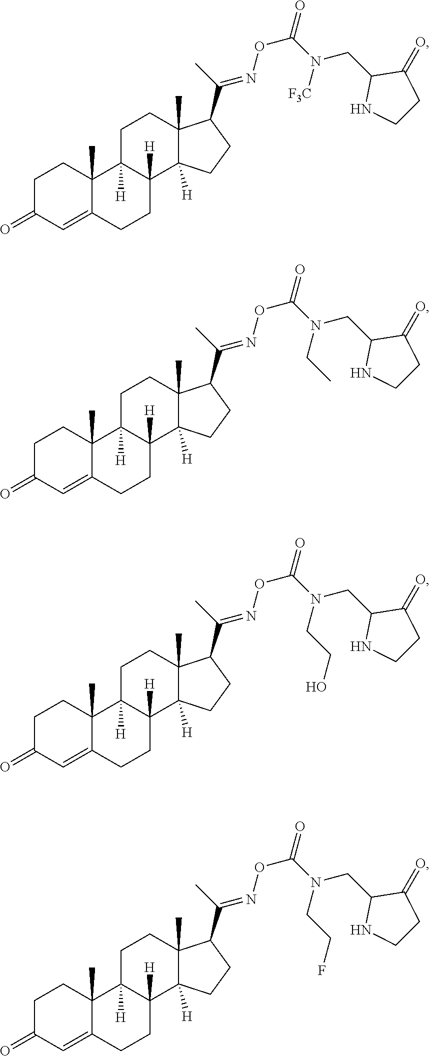

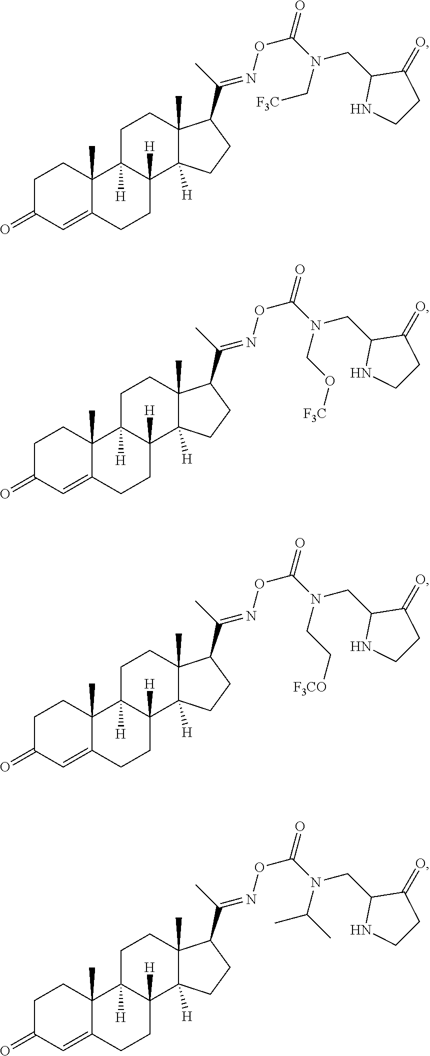

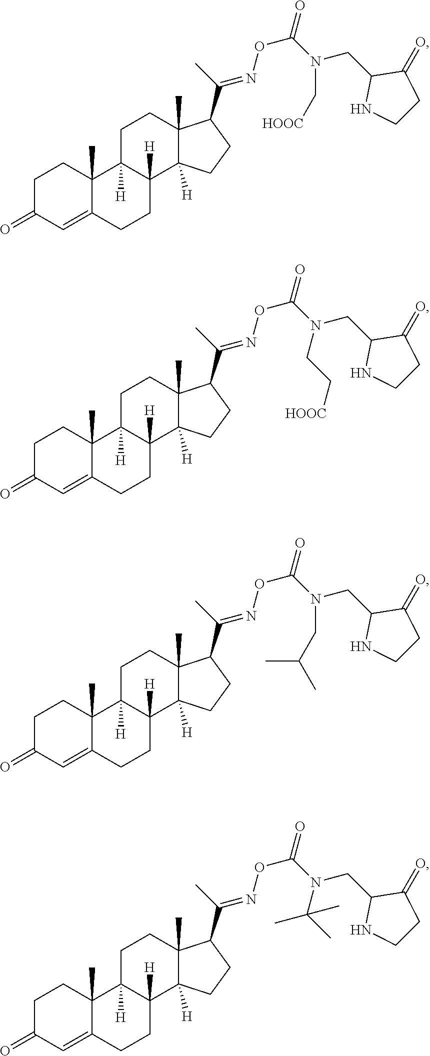

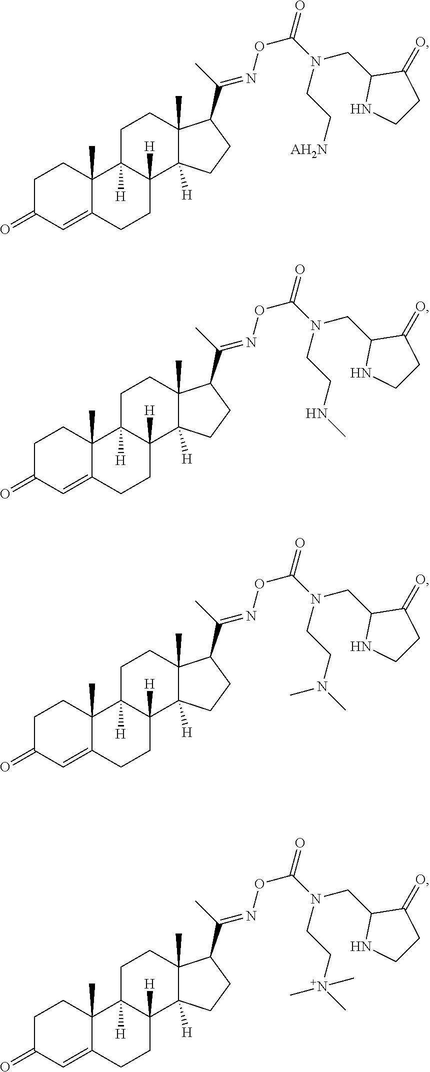

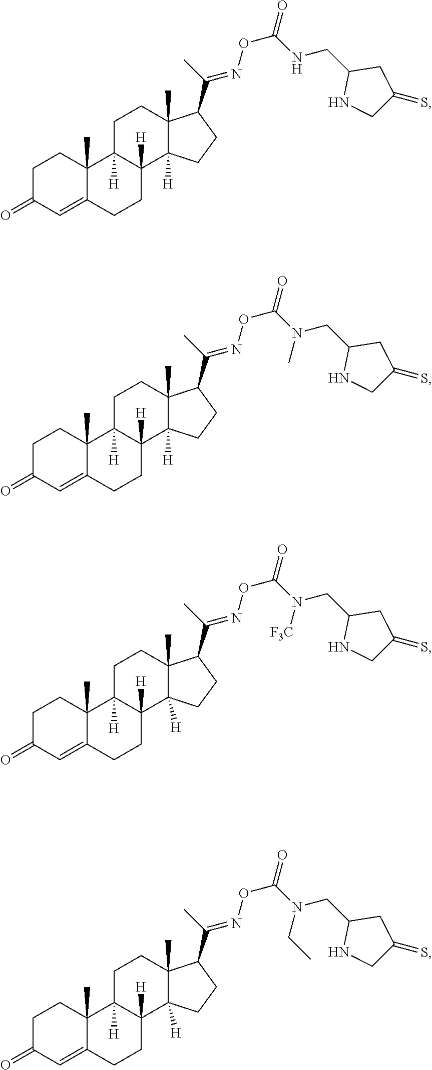

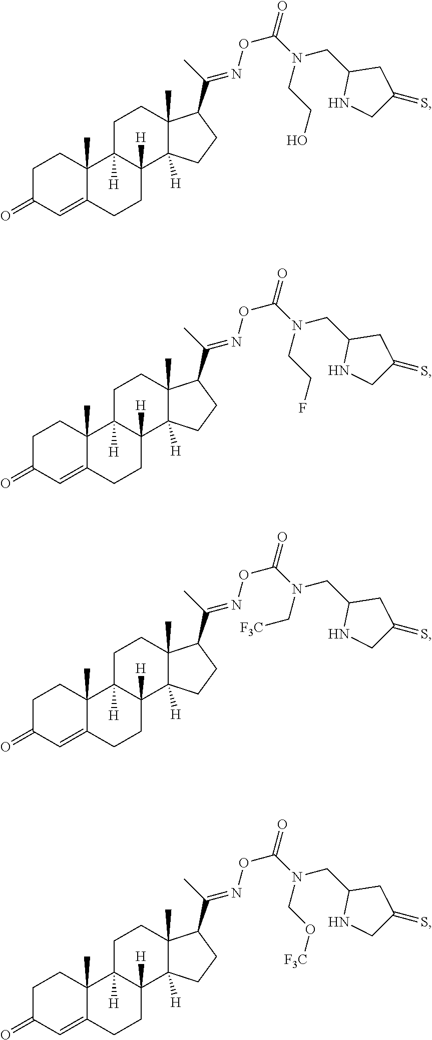

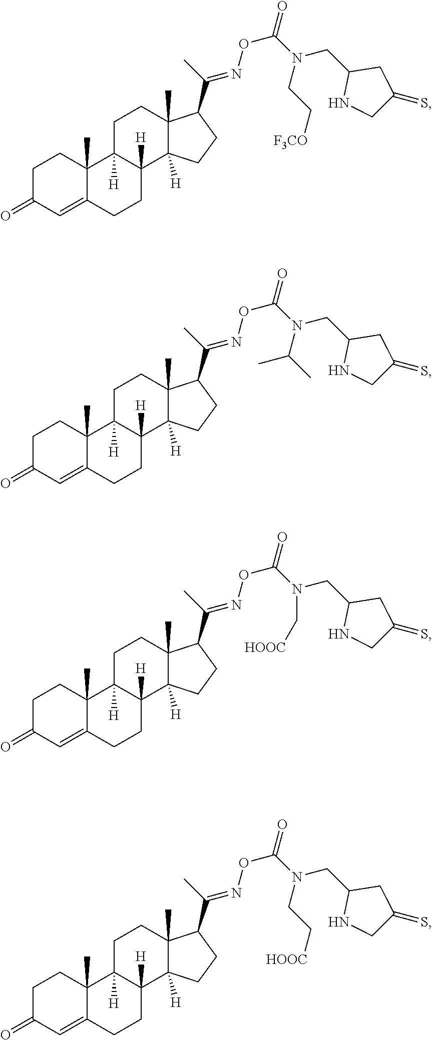

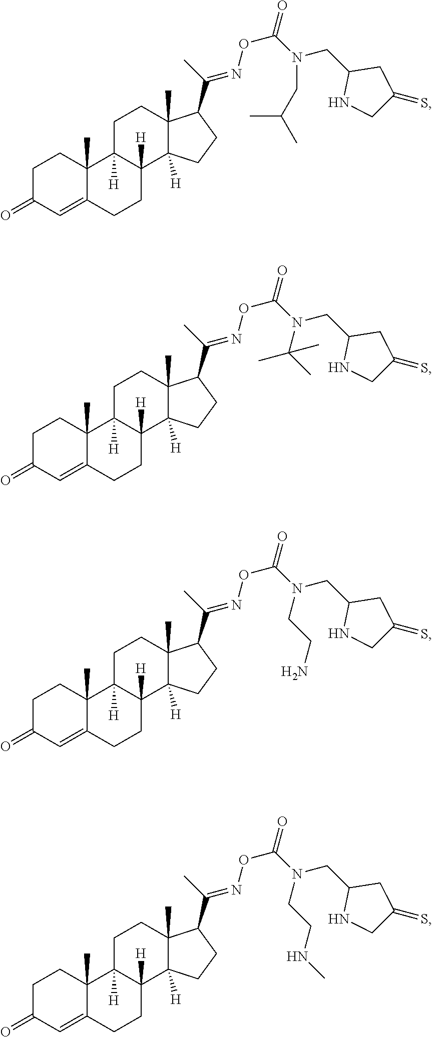

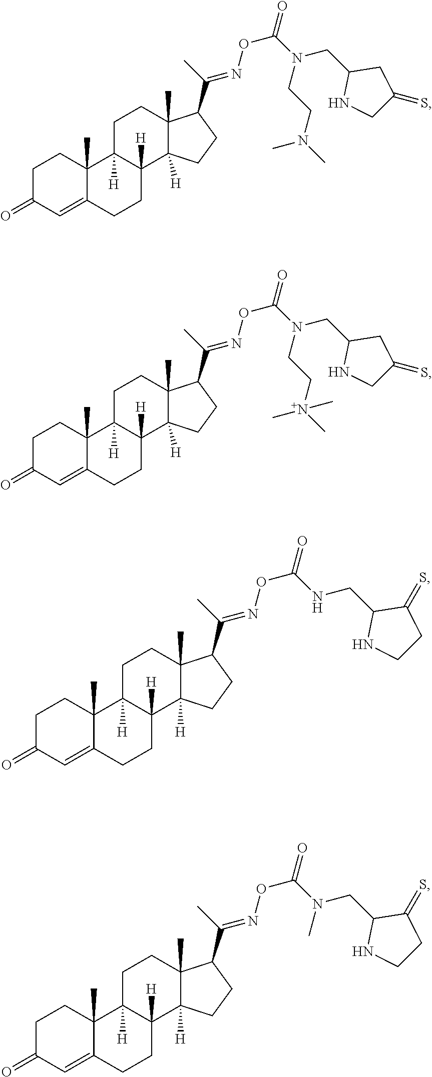

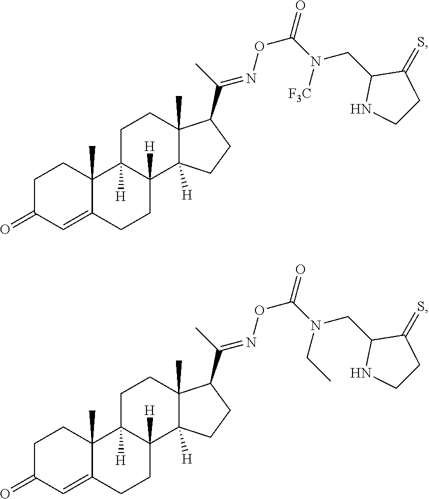

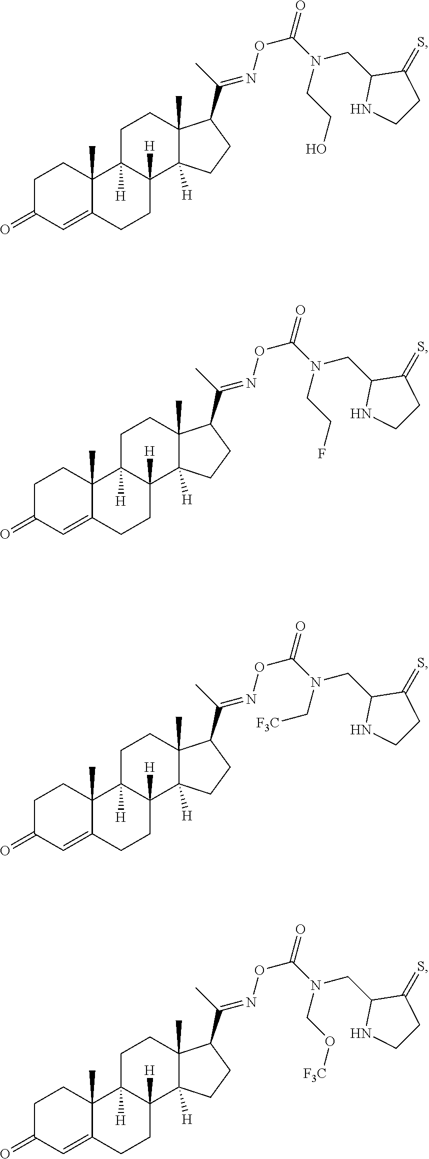

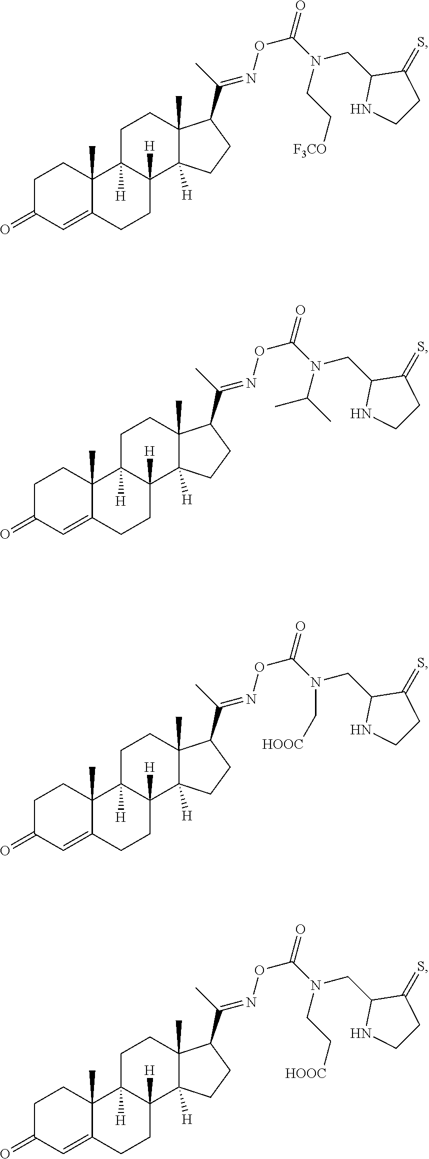

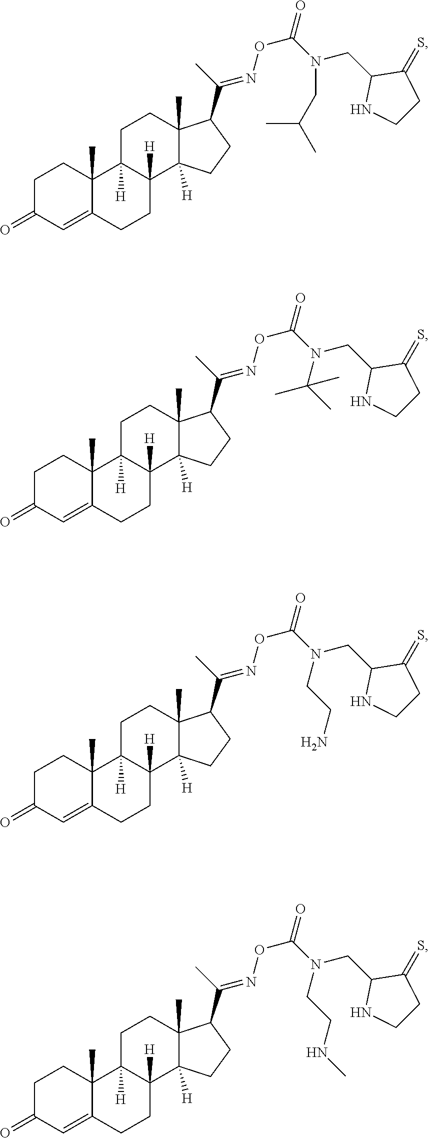

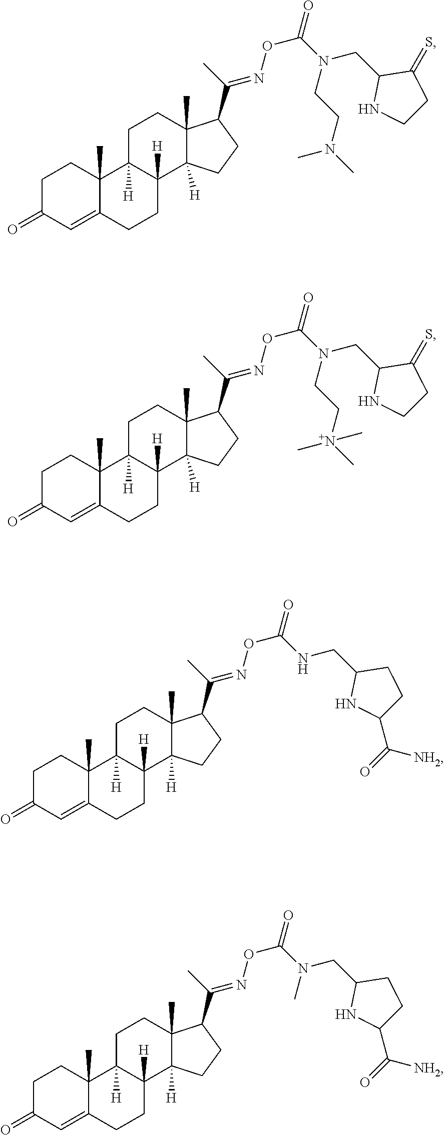

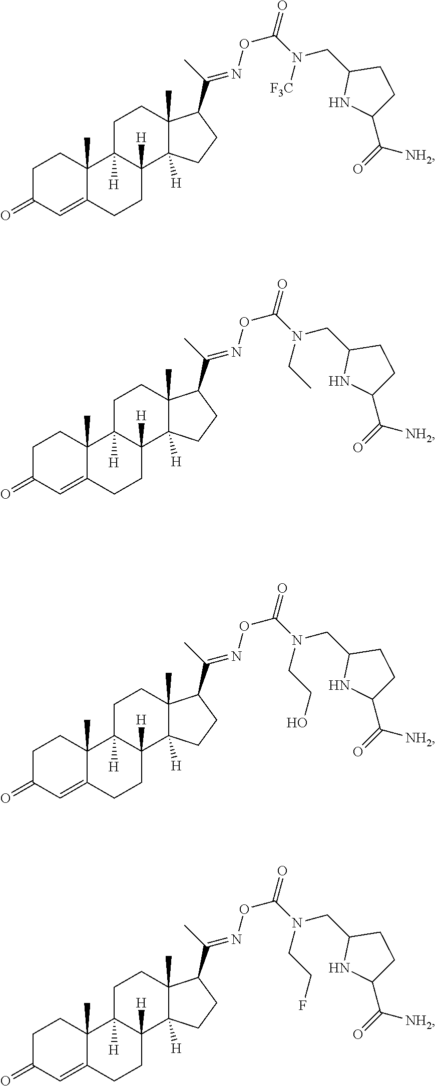

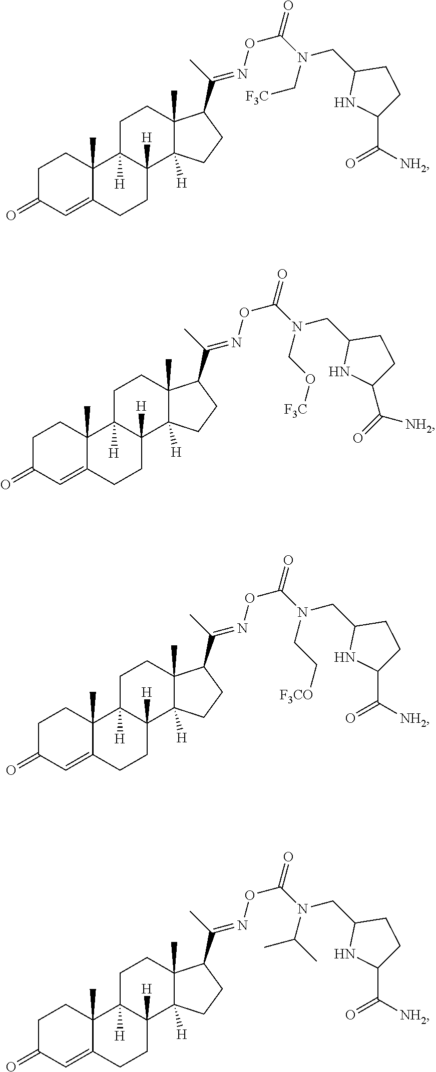

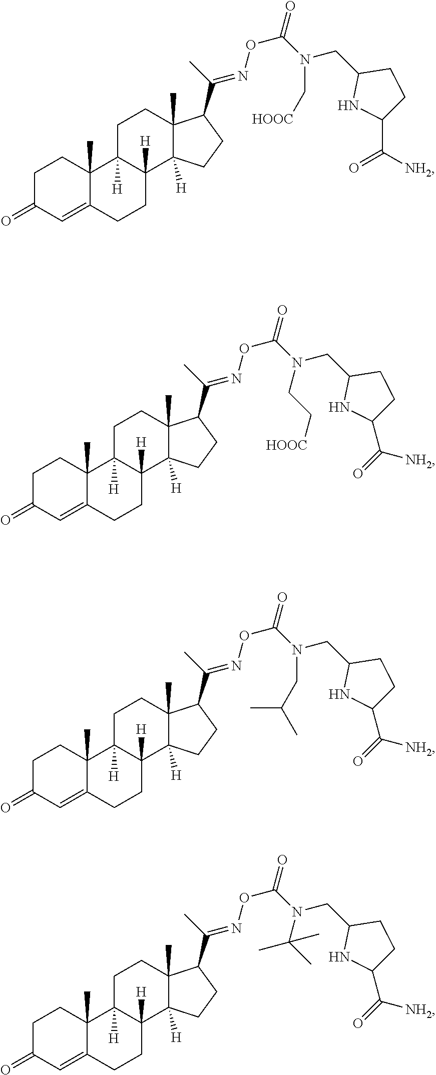

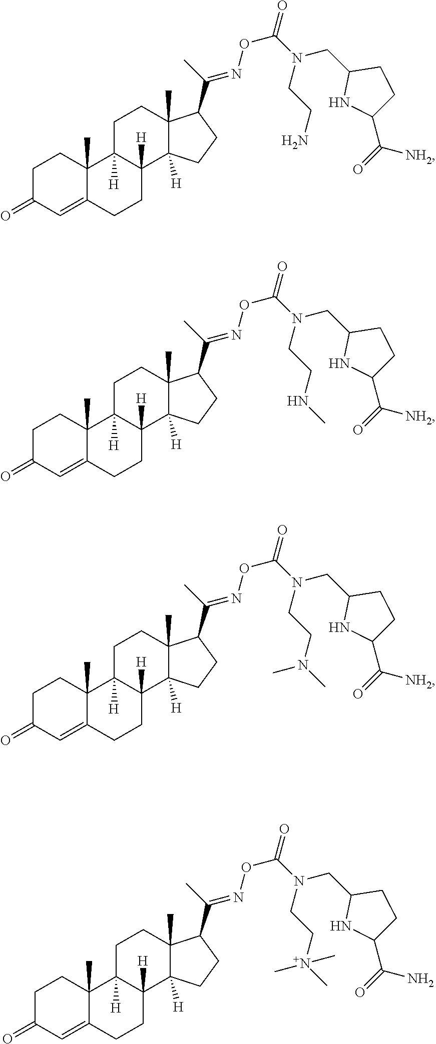

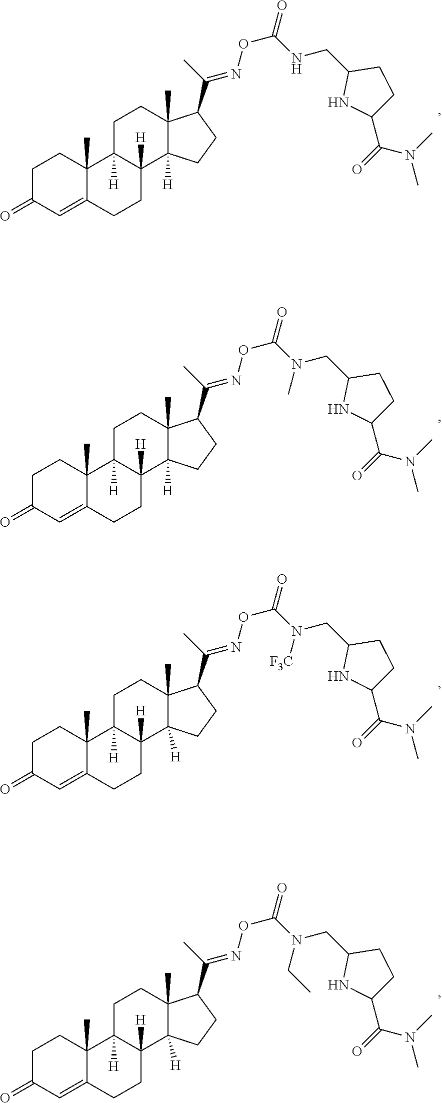

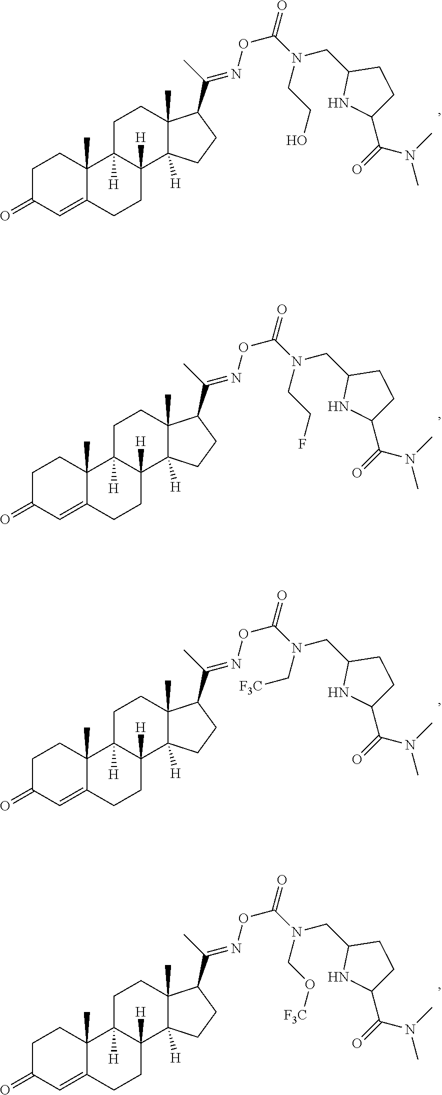

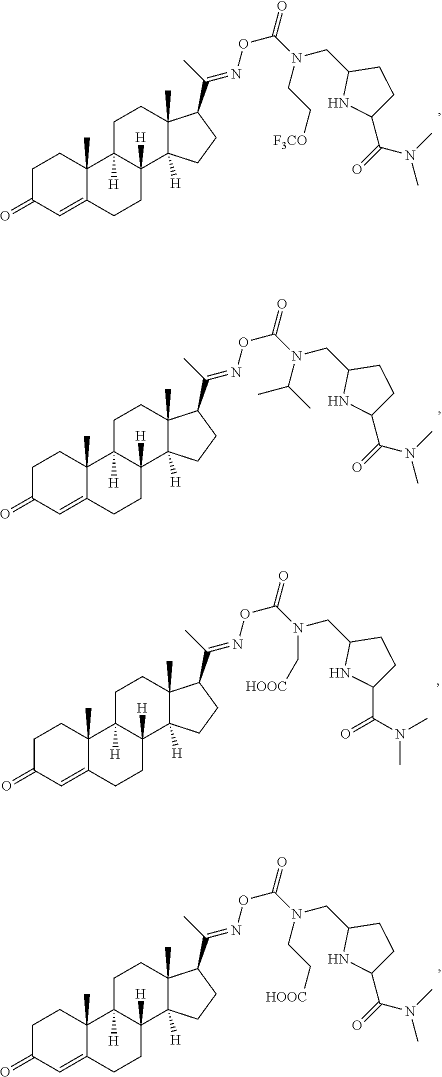

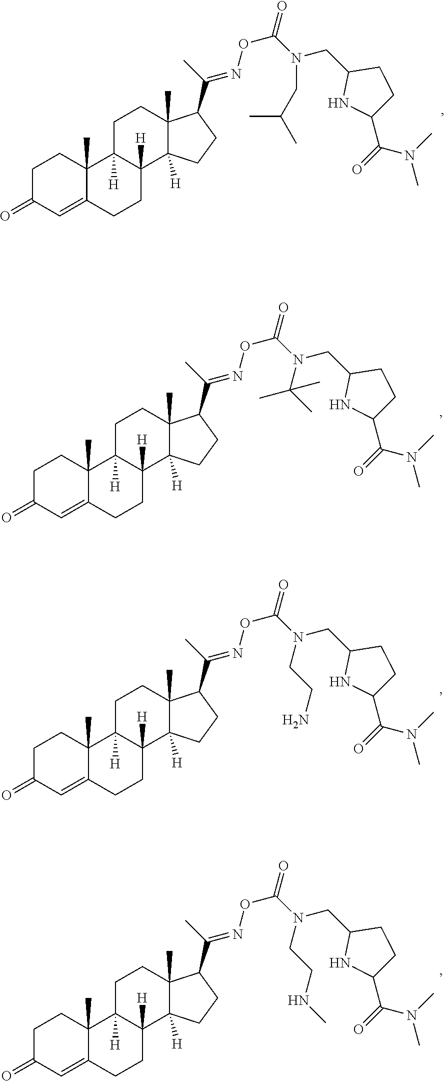

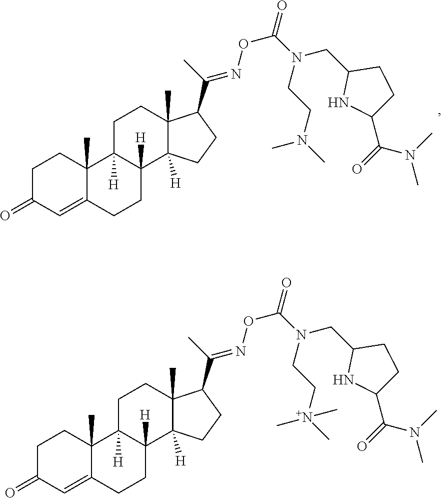

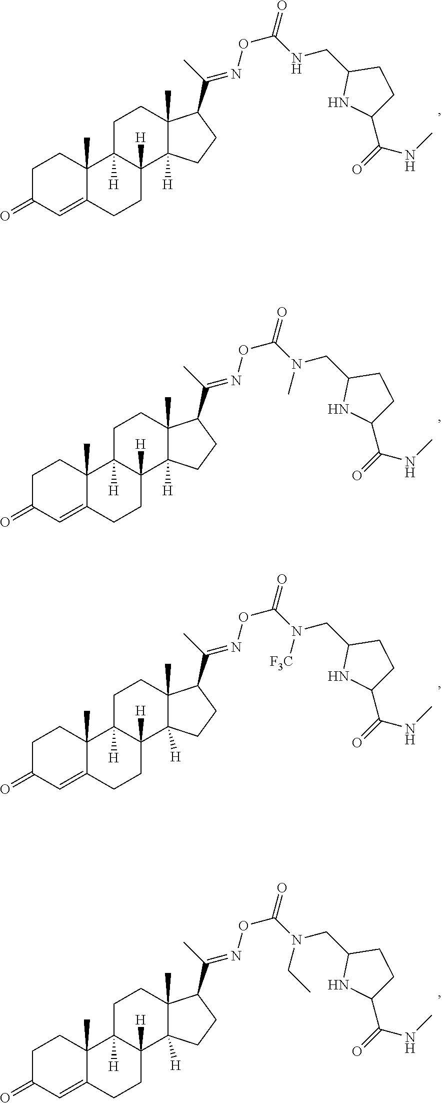

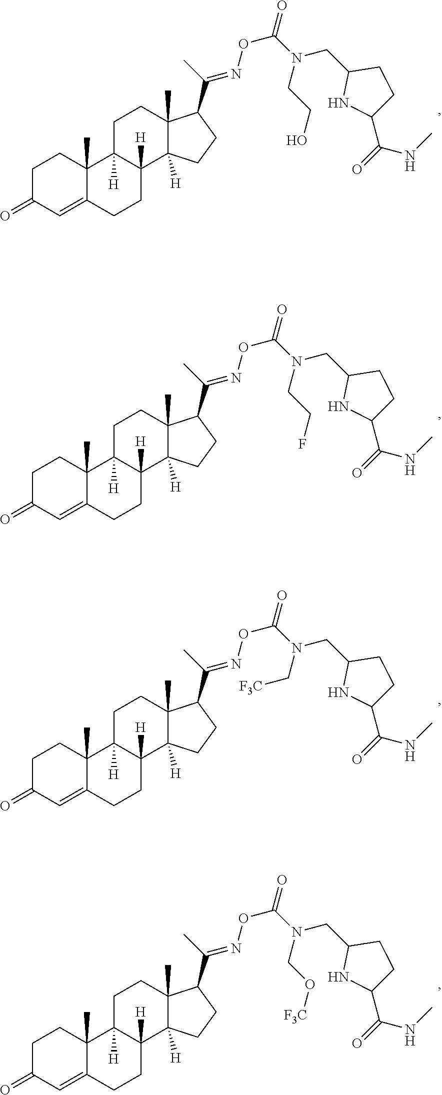

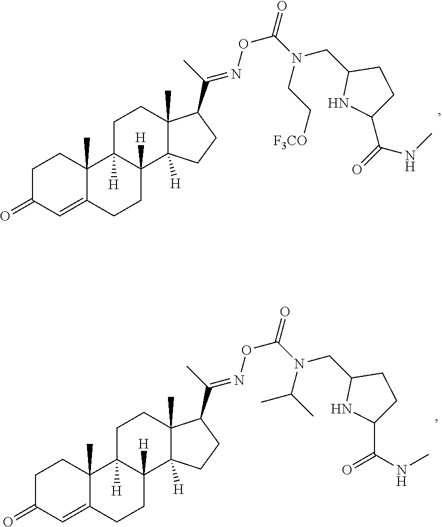

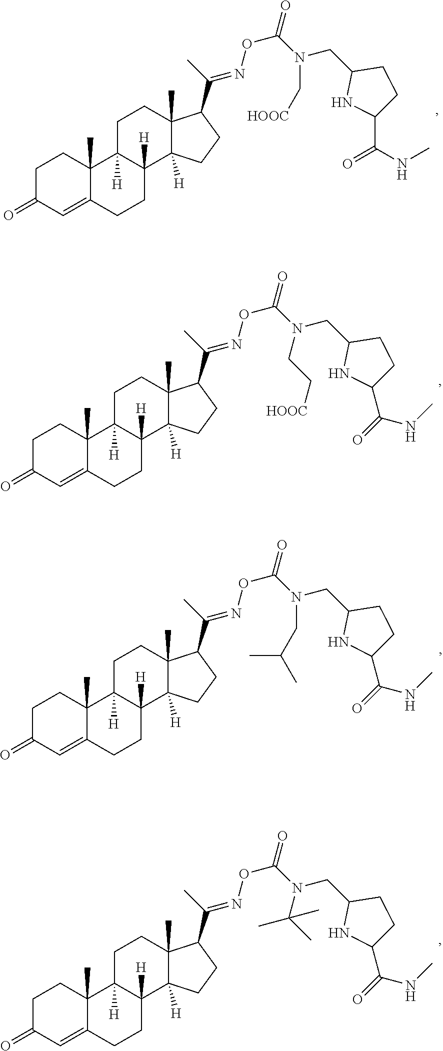

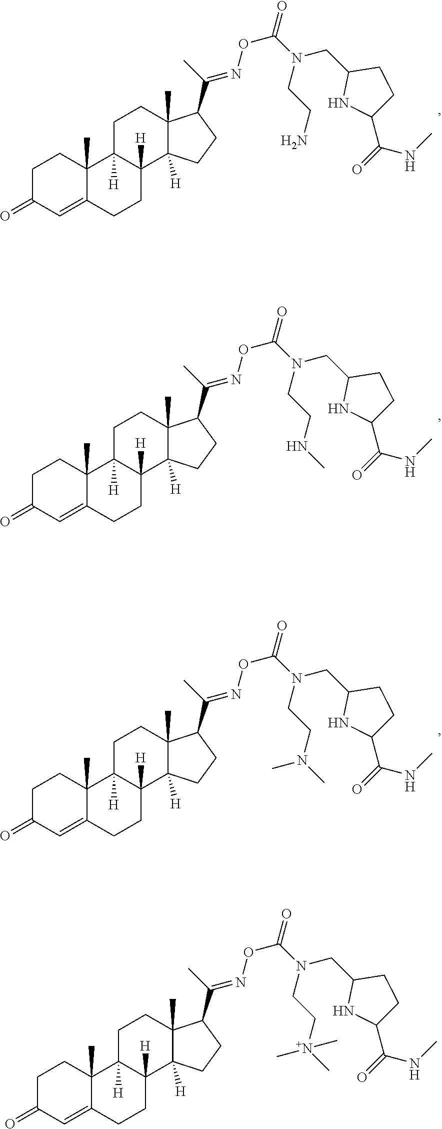

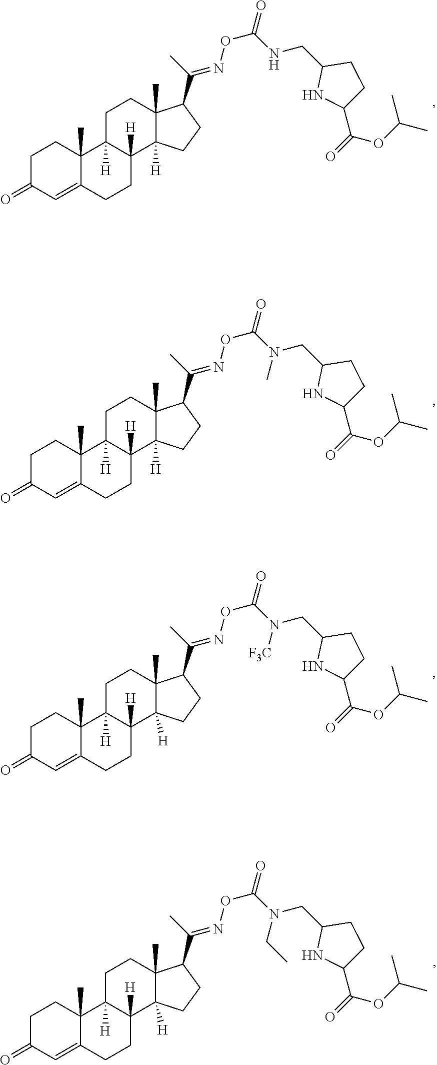

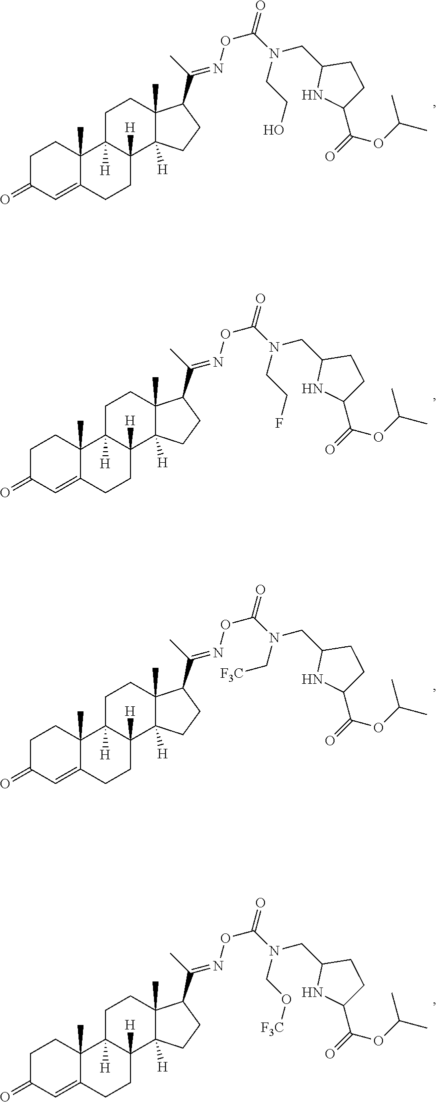

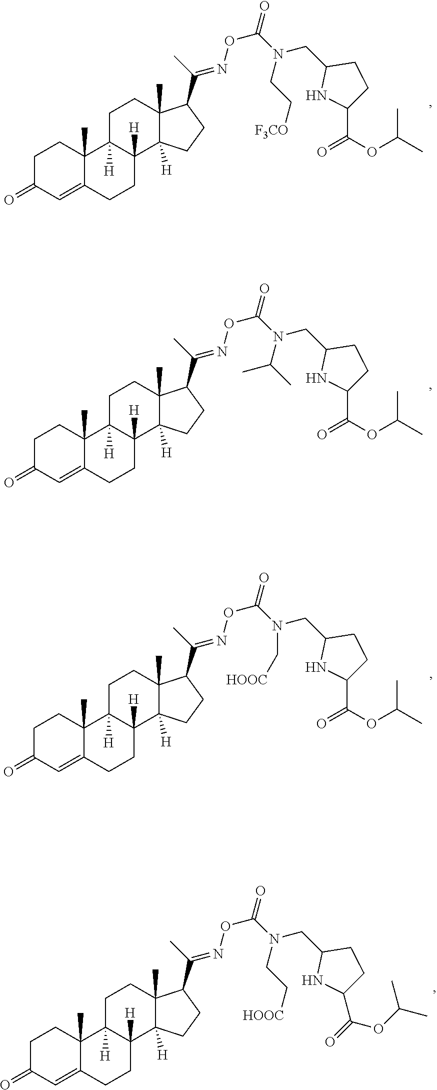

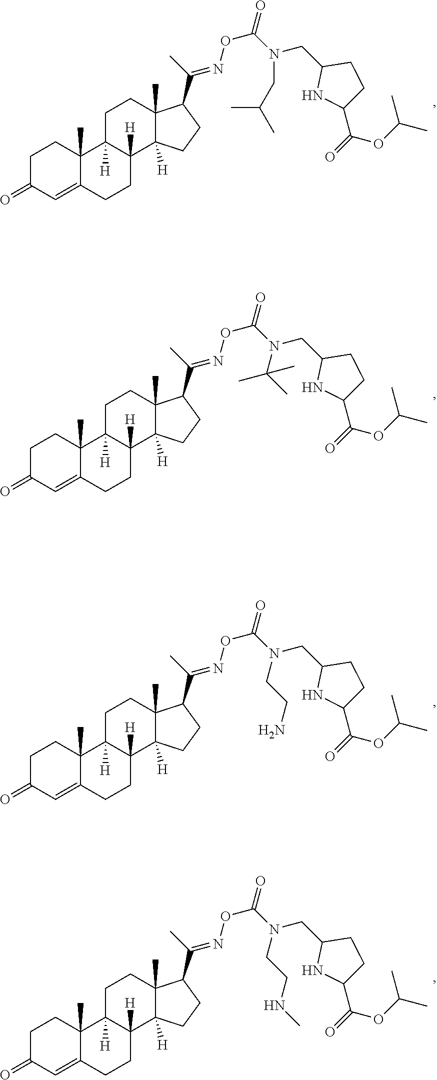

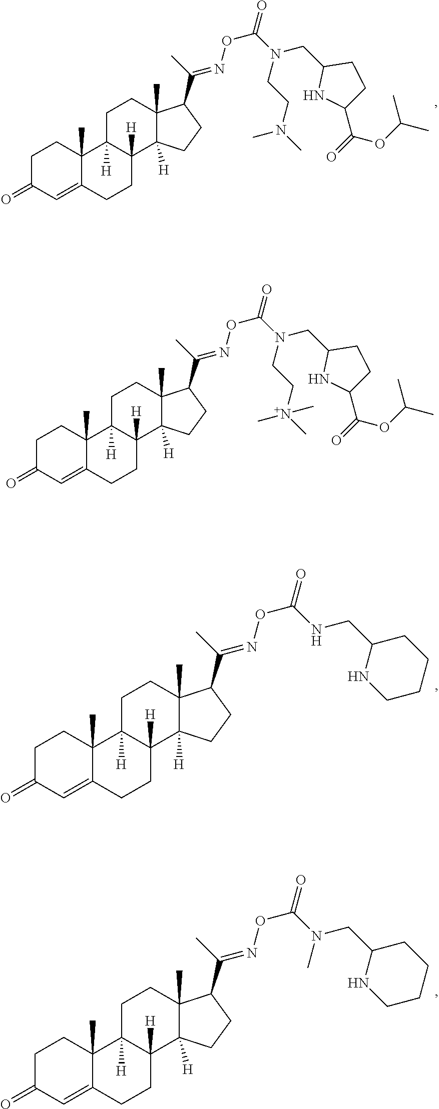

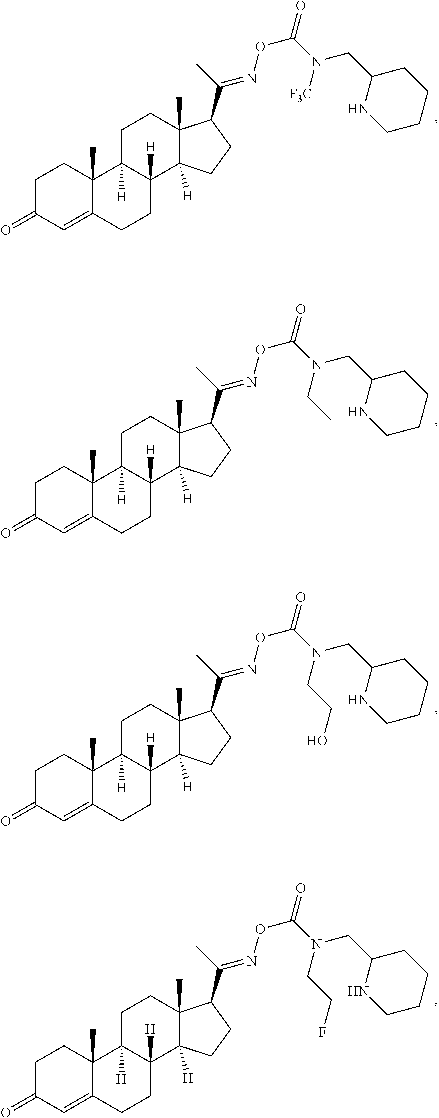

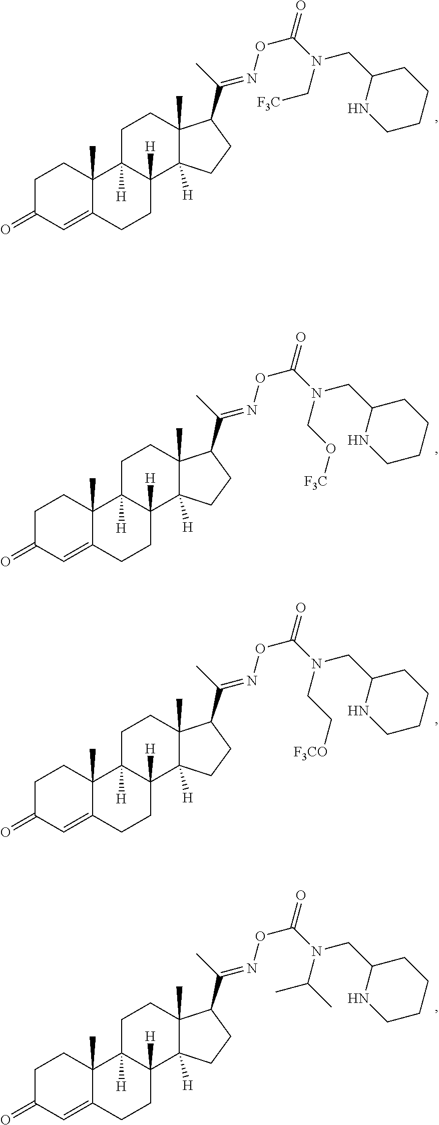

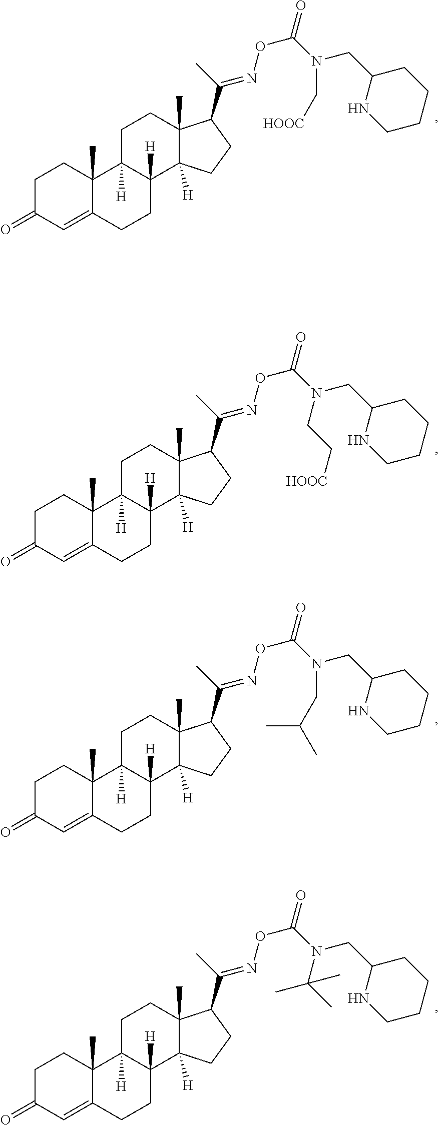

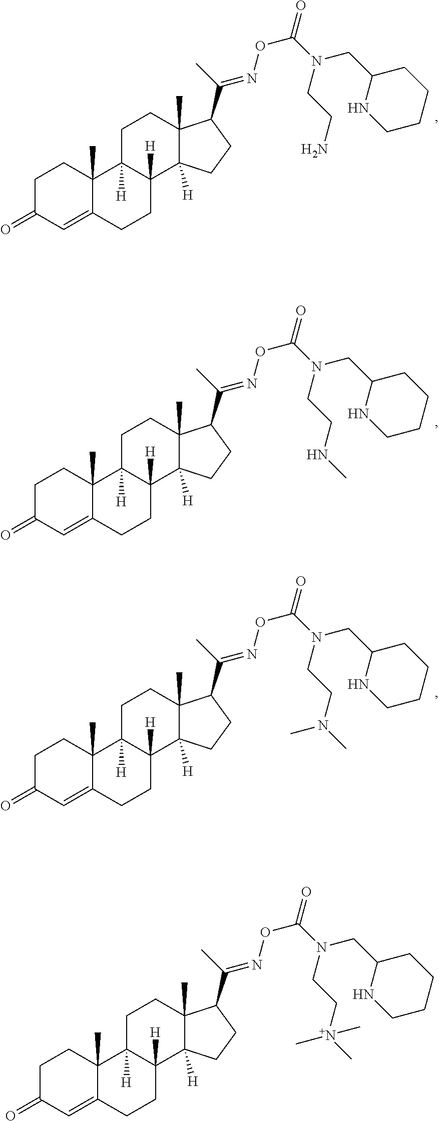

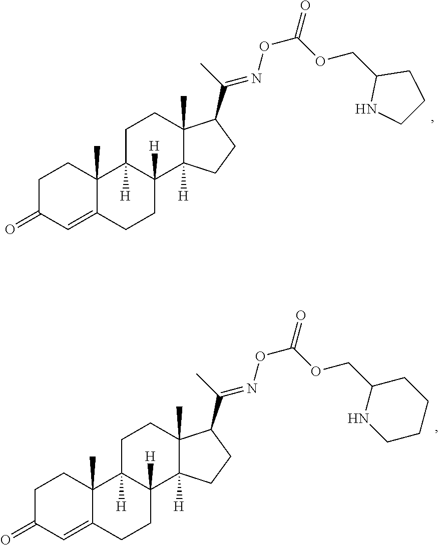

and pharmaceutically acceptable salts thereof. In some embodiments, the ring carbon atom in the pyrrolidine or piperidine moiety that connects to the rest of the structure is in the S configuration. In some embodiments, the ring carbon atom in the pyrrolidine or piperidine moiety that connects to the rest of the structure is in the R configuration.

Exemplary compounds of Formula II-1 also include:

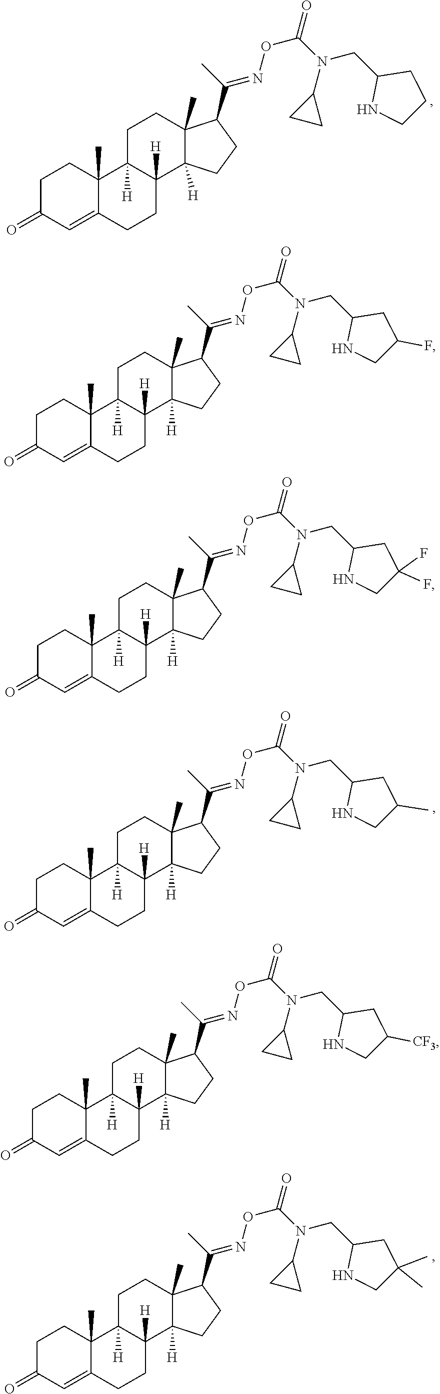

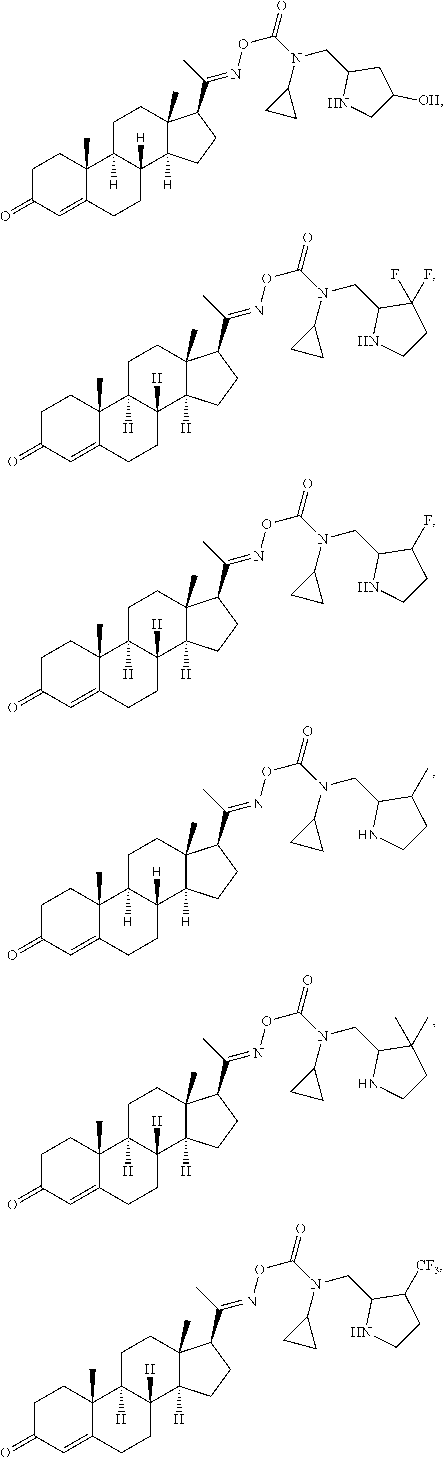

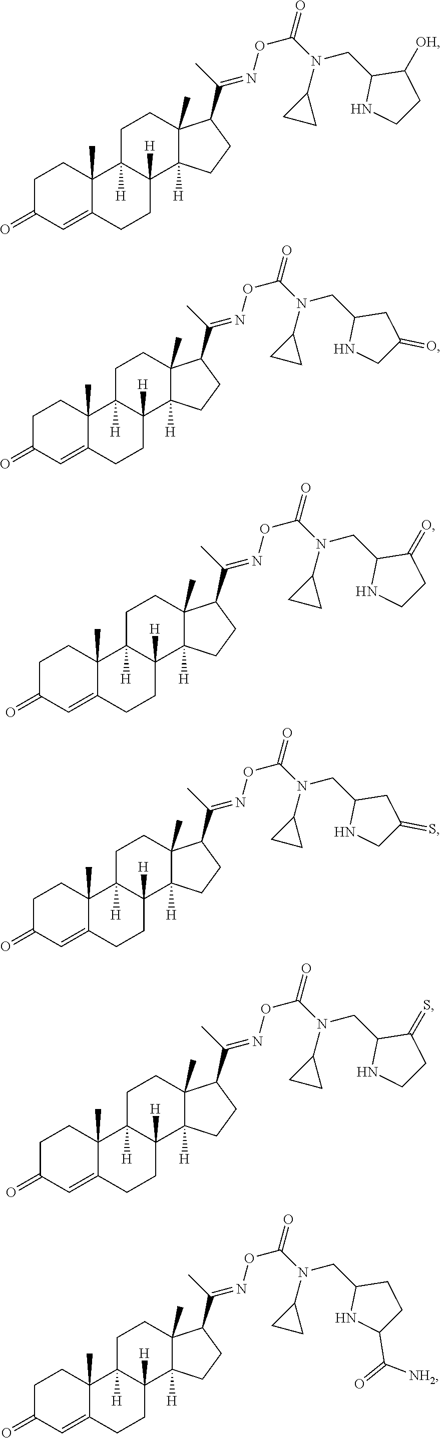

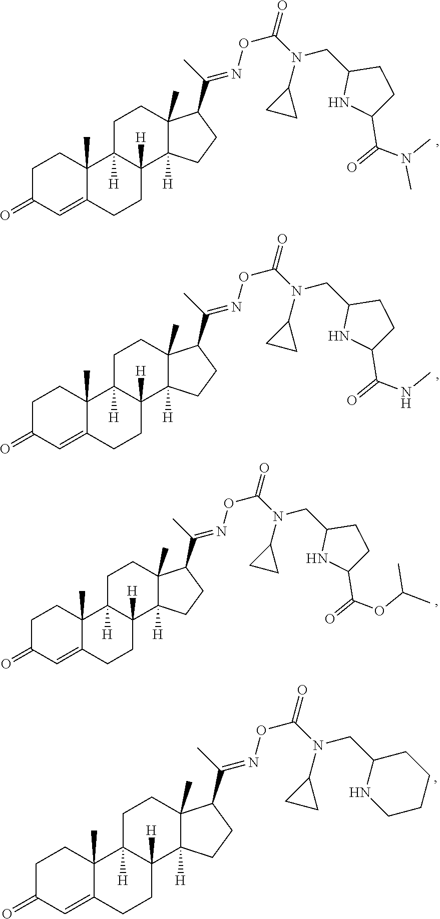

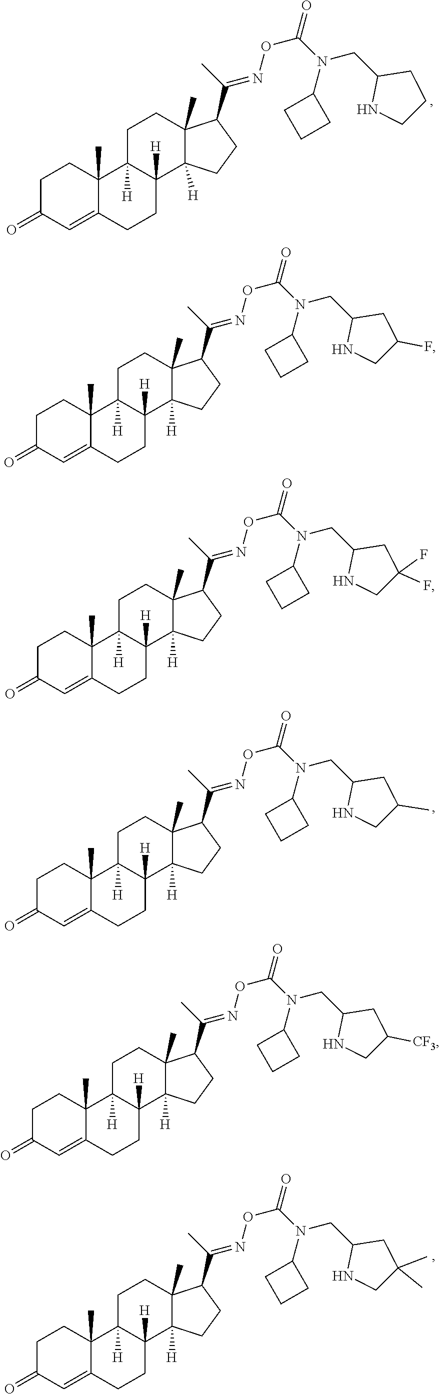

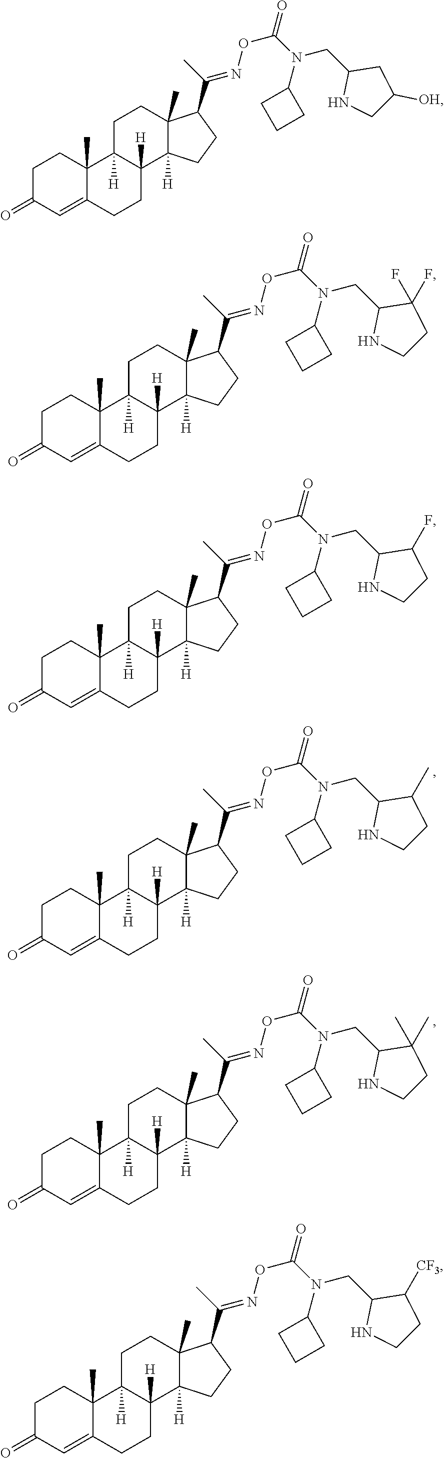

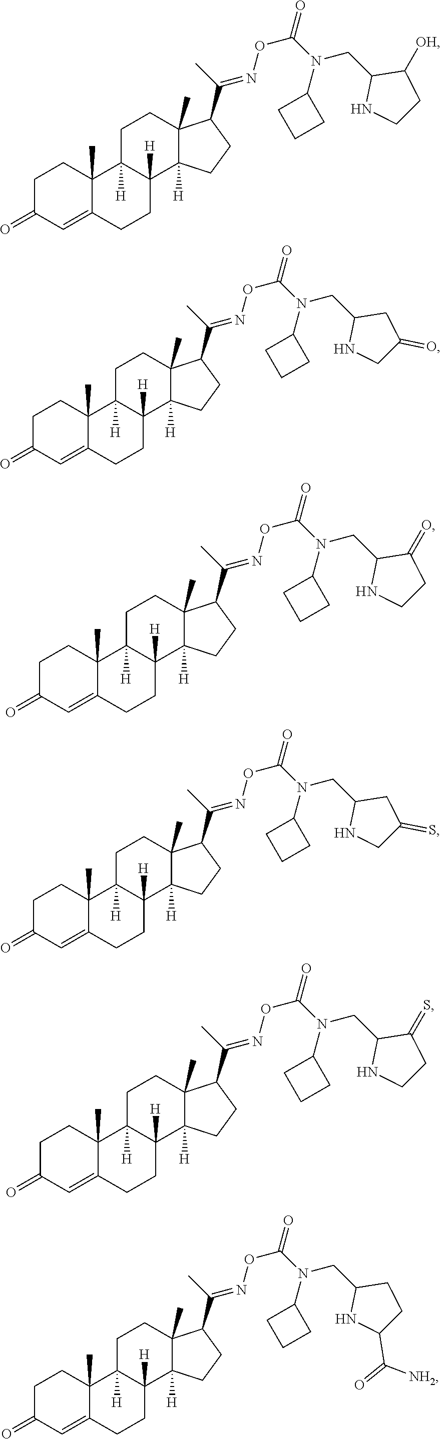

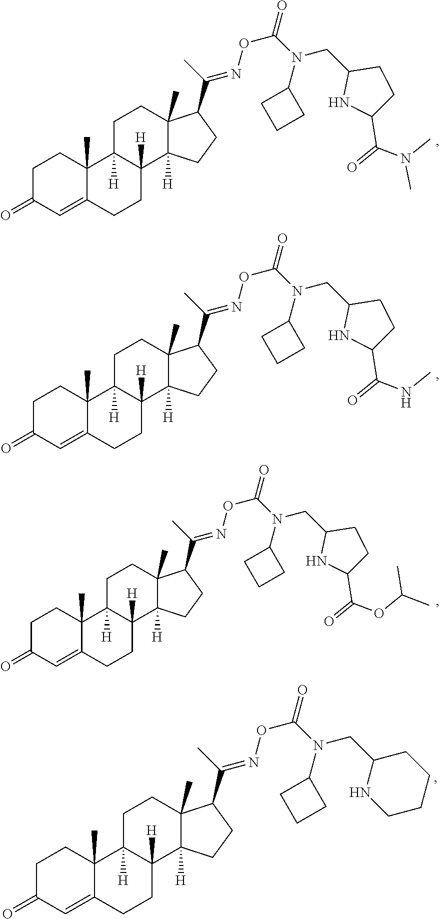

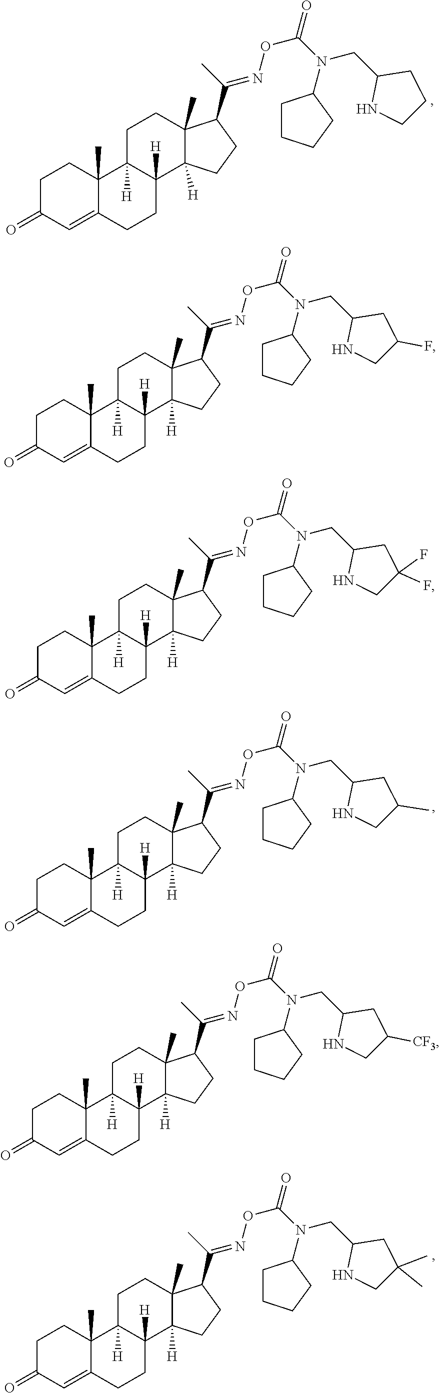

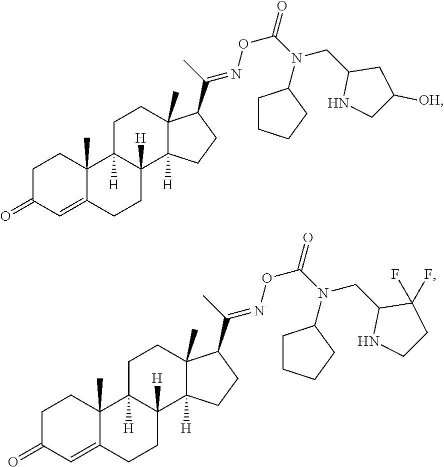

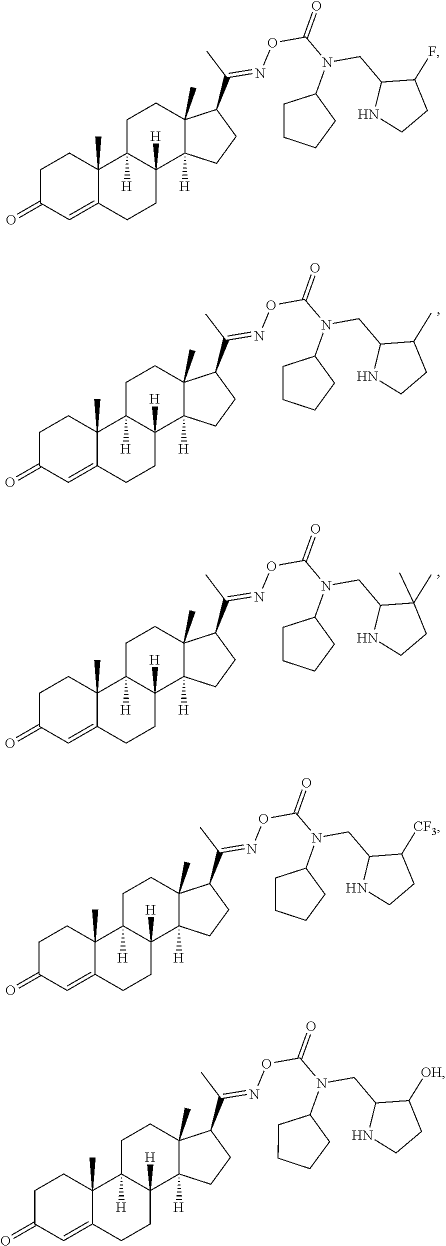

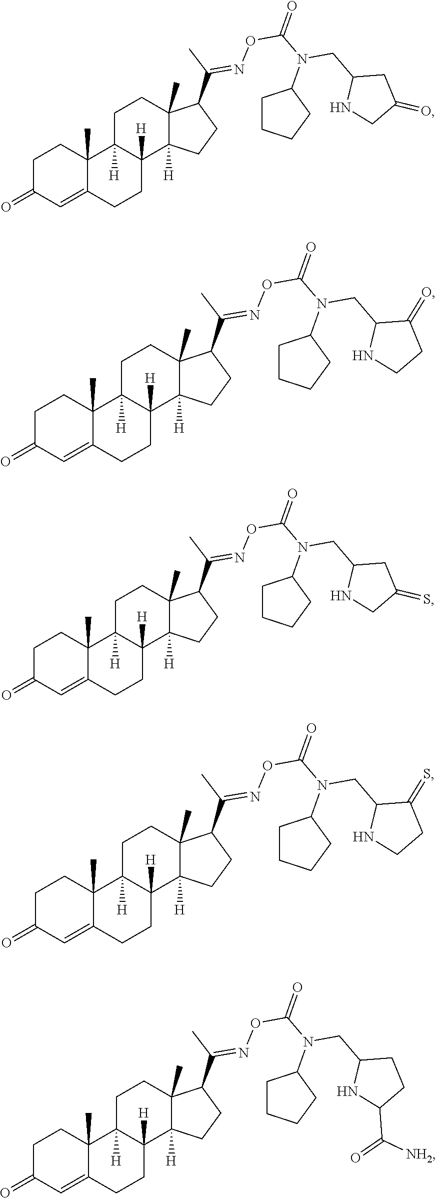

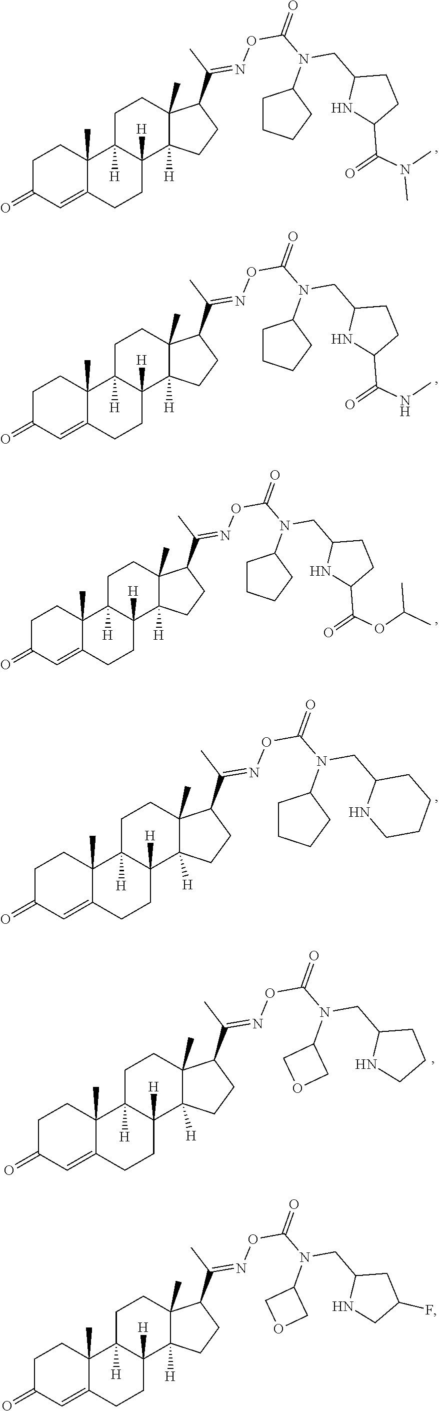

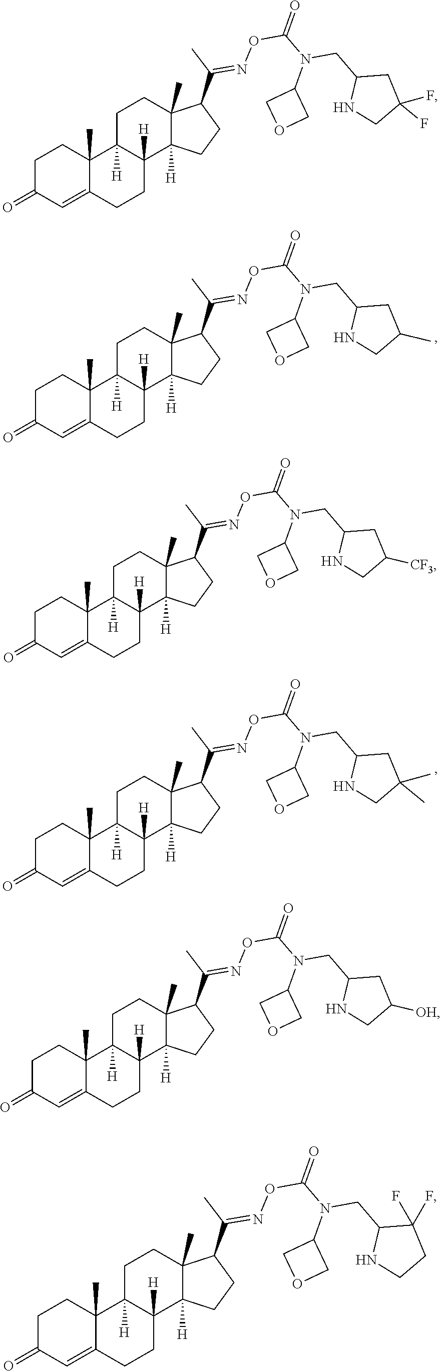

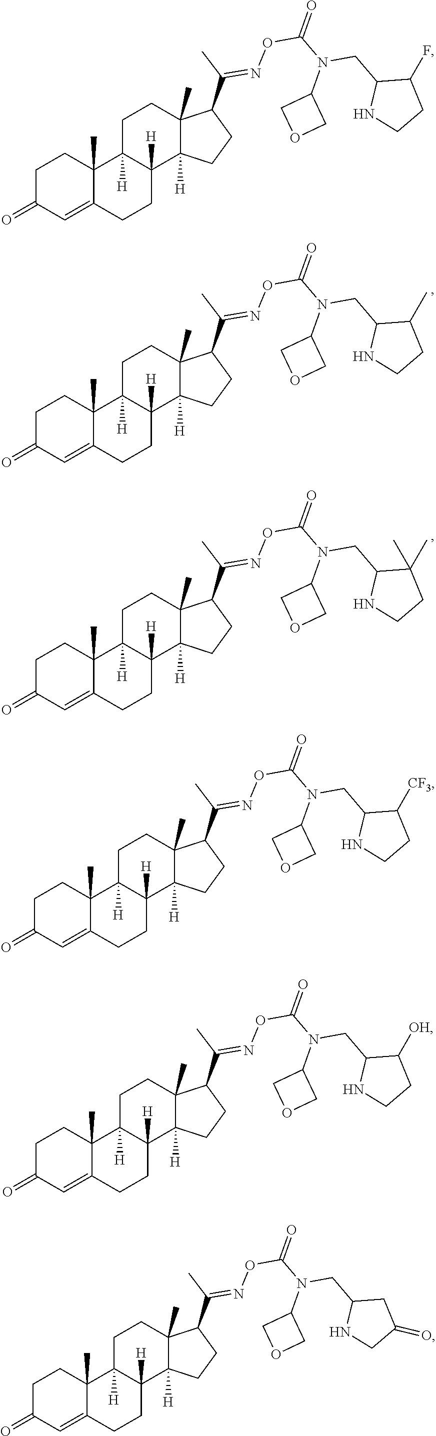

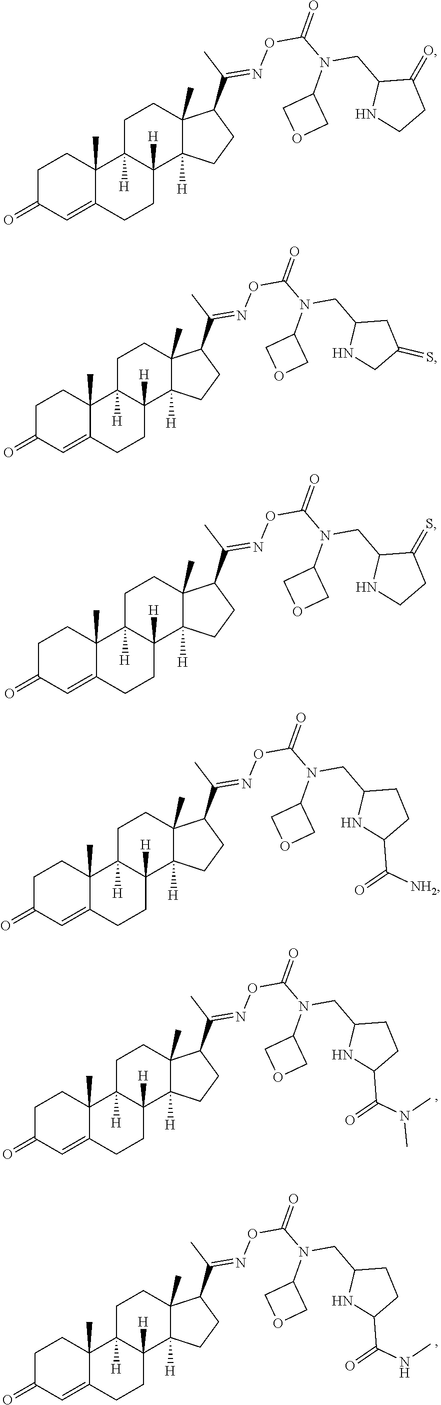

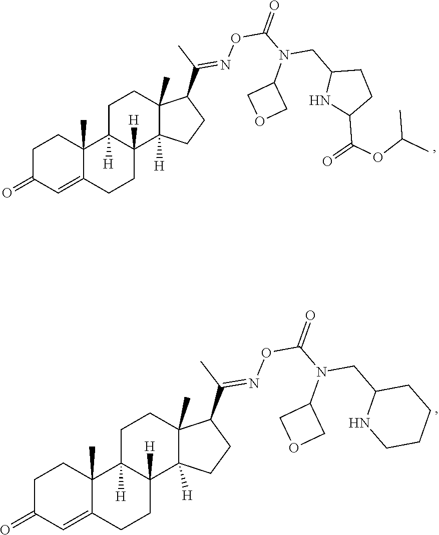

and pharmaceutically acceptable salts thereof. In some embodiments, the ring carbon atom in the pyrrolidine or piperidine moiety that connects to the rest of the structure is in the S configuration. In some embodiments, the ring carbon atom in the pyrrolidine or piperidine moiety that connects to the rest of the structure is in the R configuration.

Exemplary compounds of Formula II-1 also include:

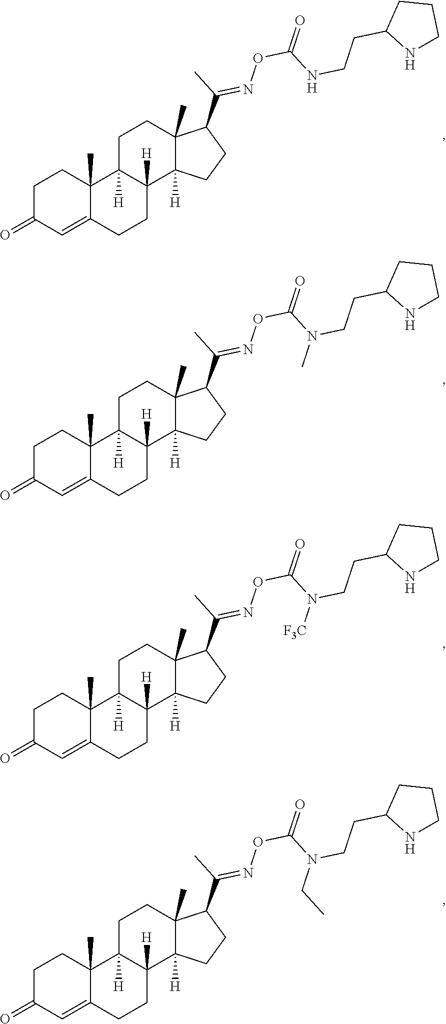

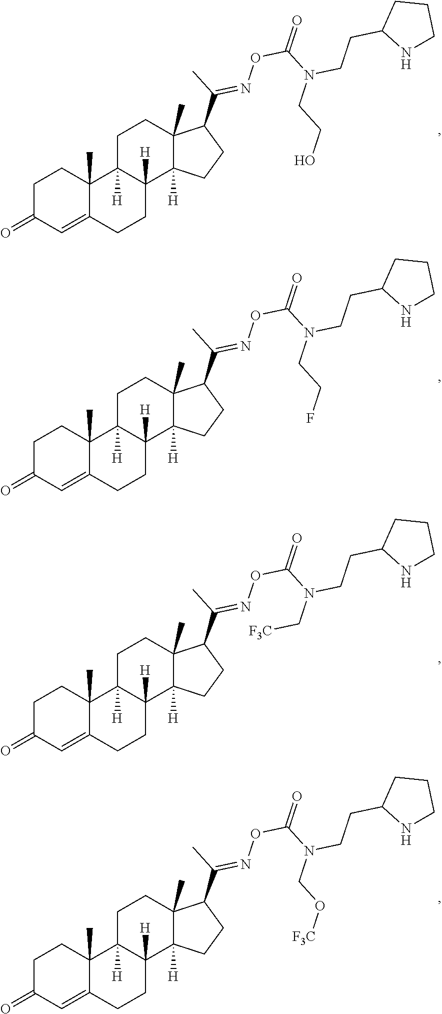

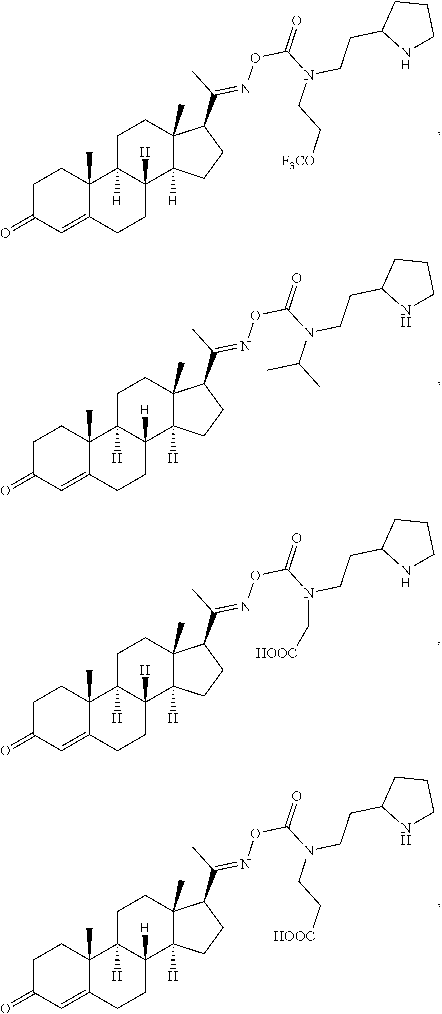

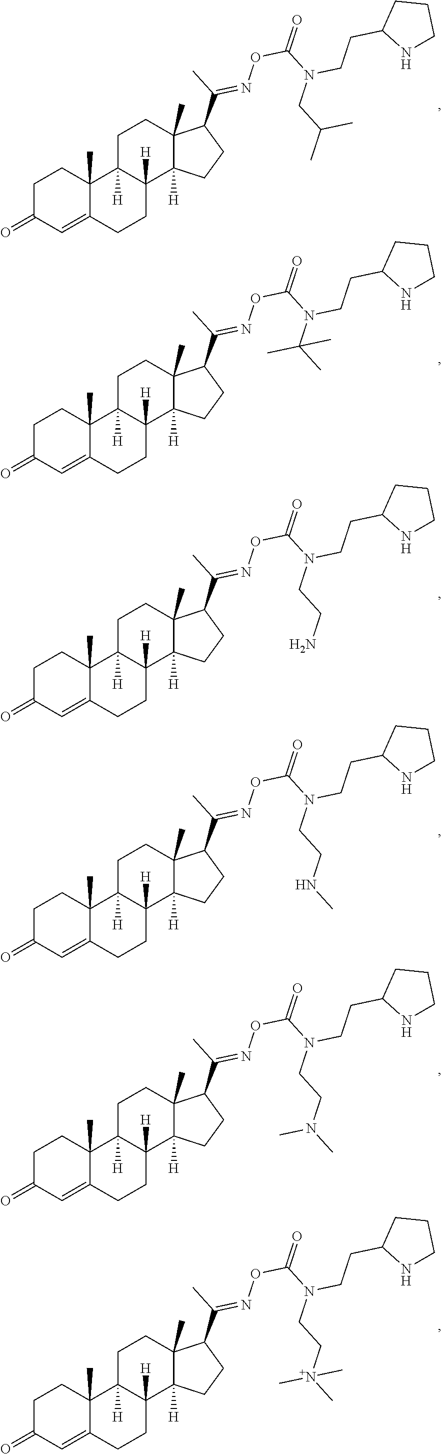

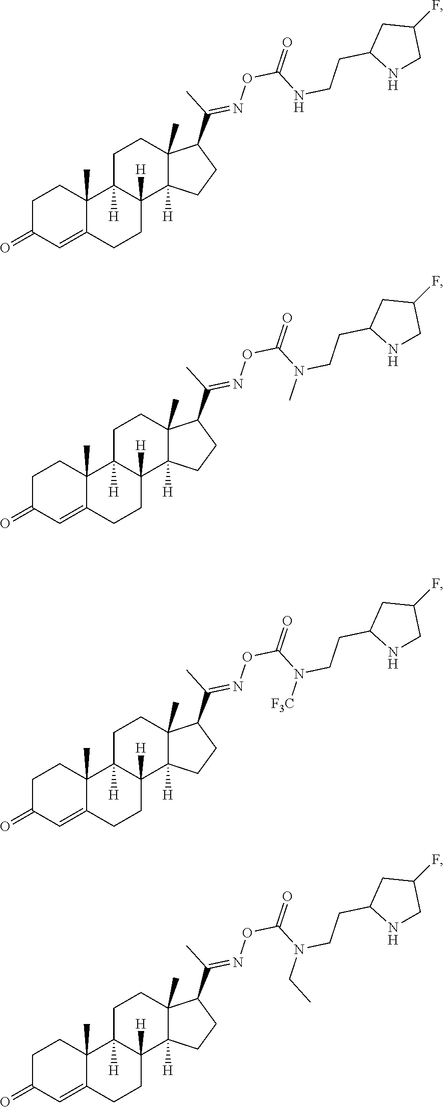

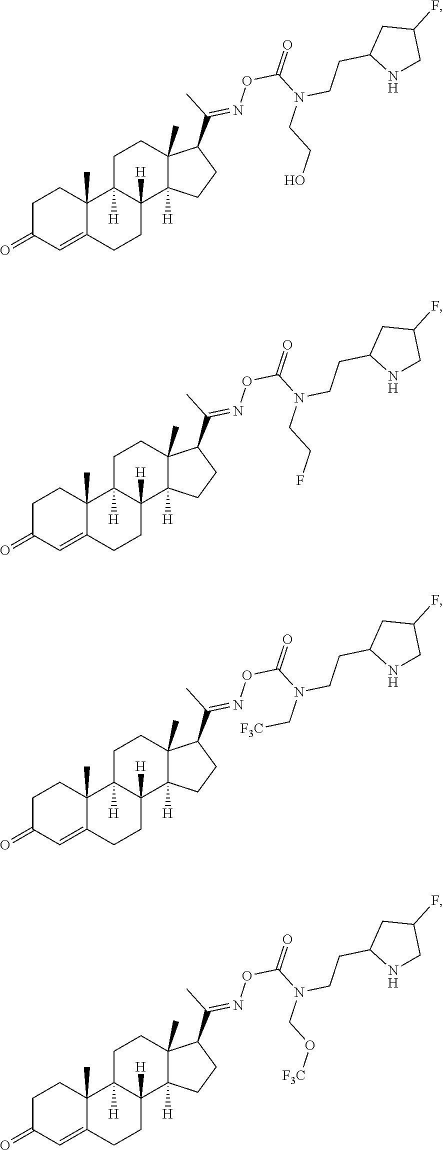

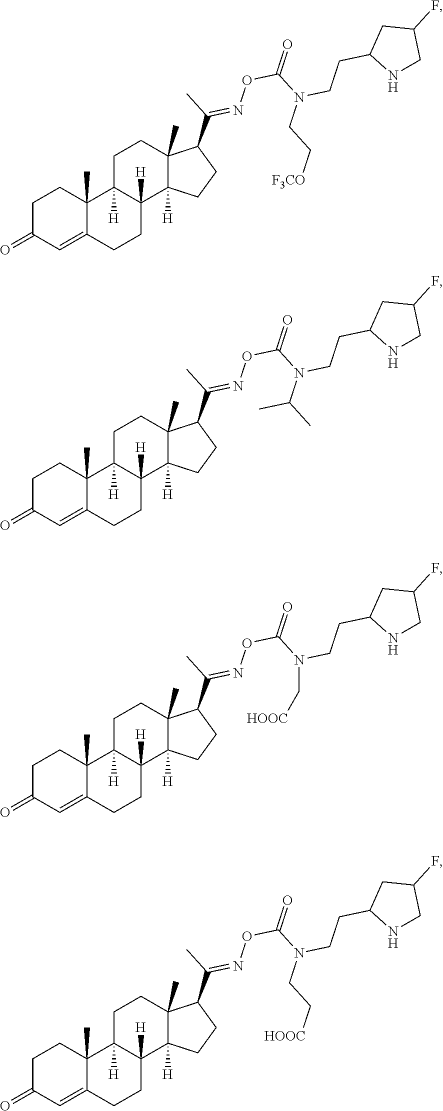

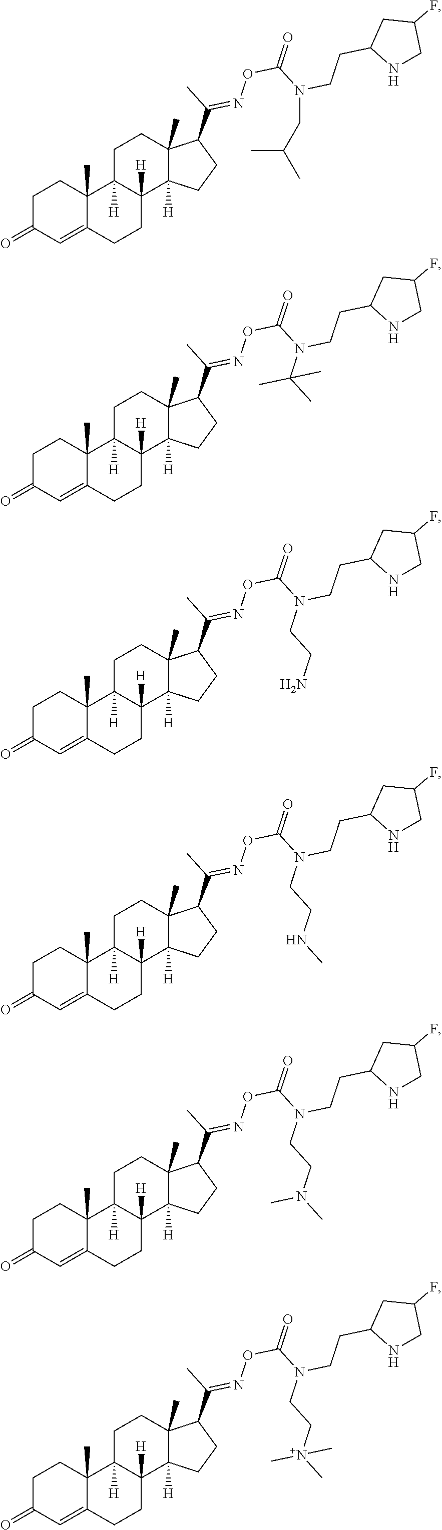

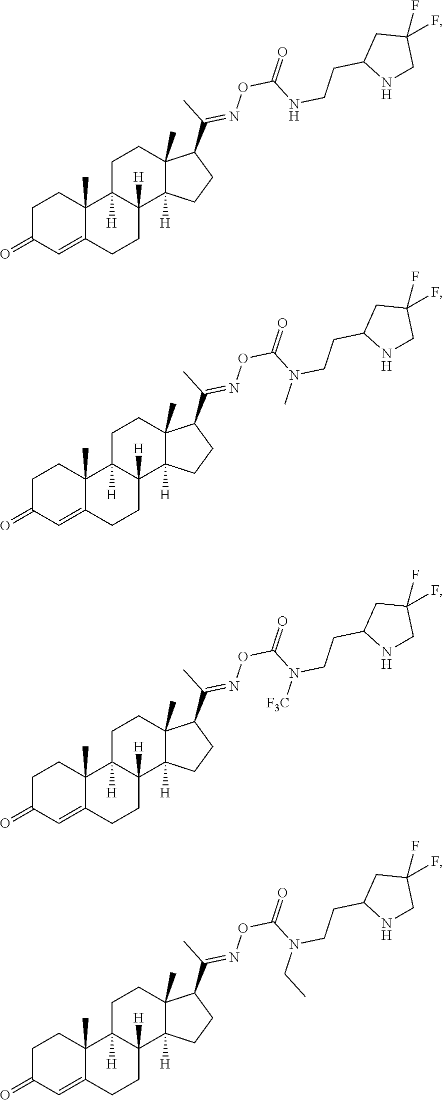

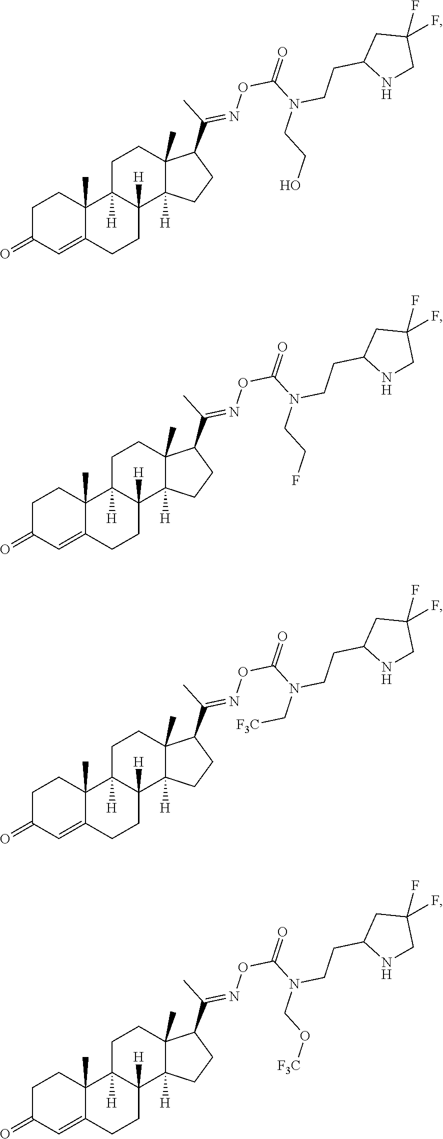

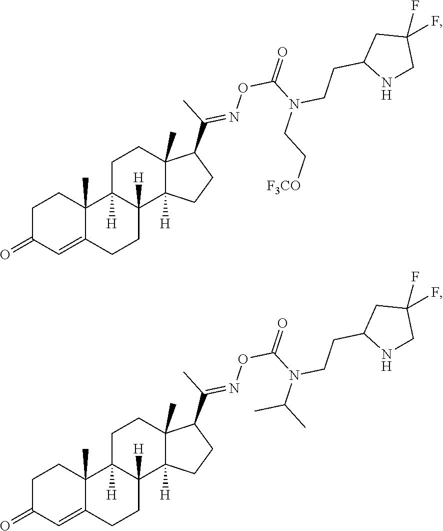

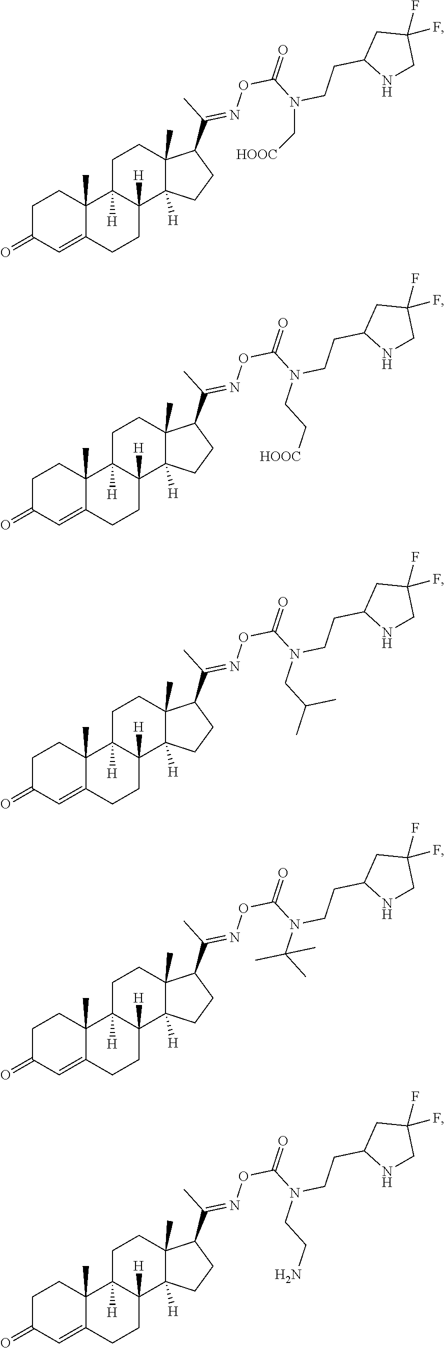

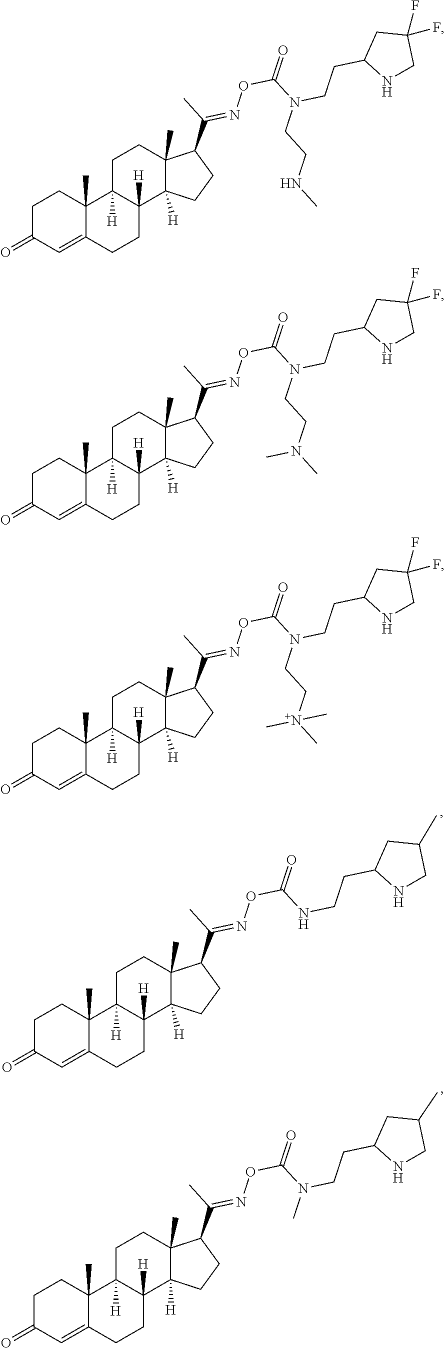

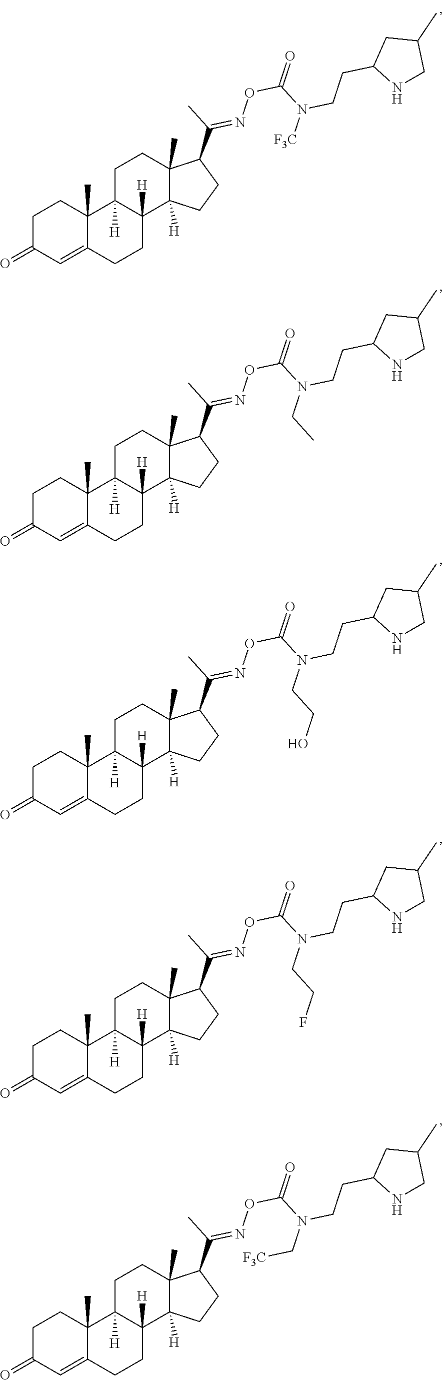

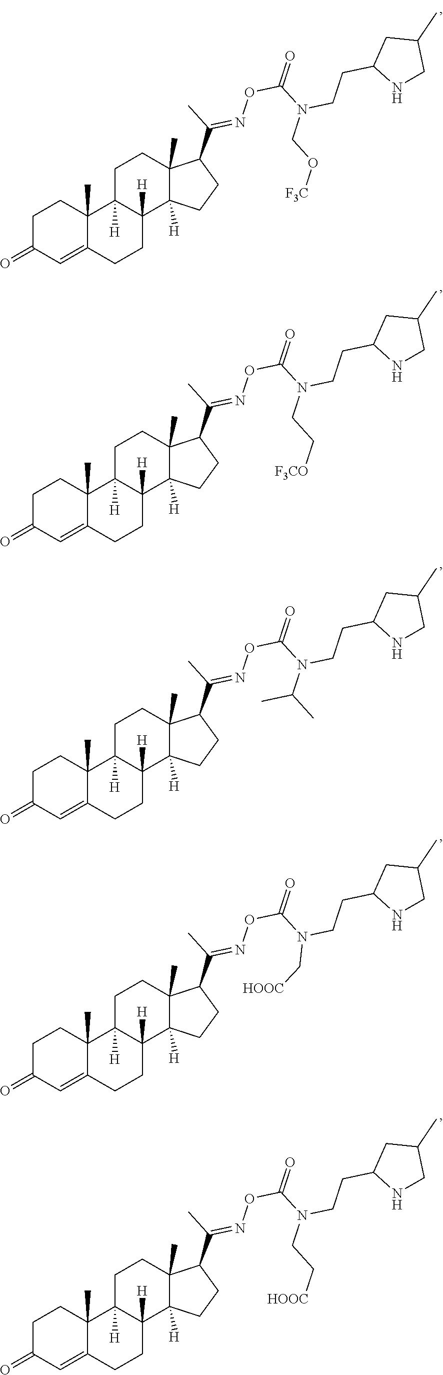

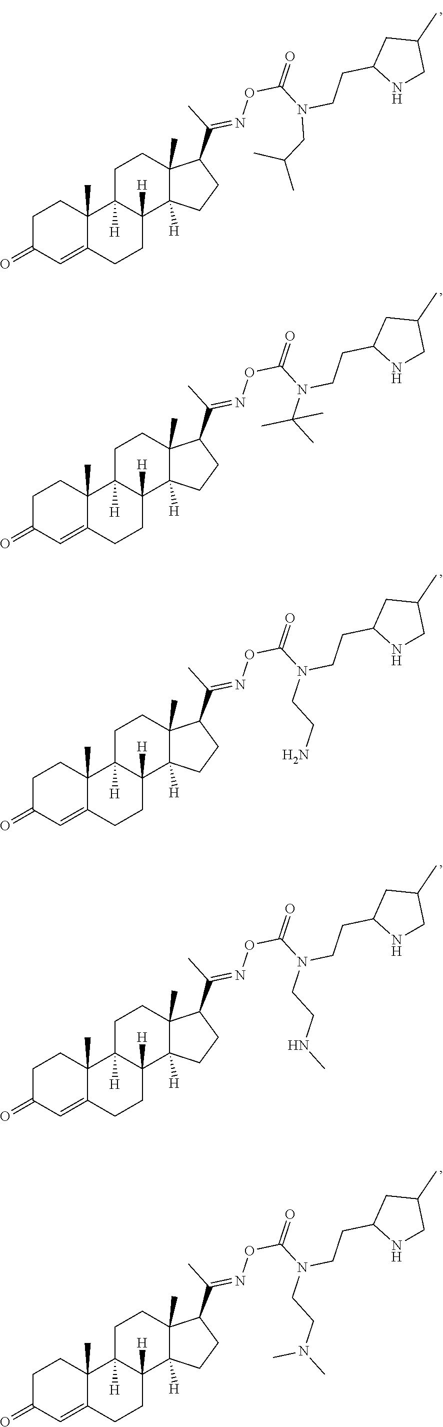

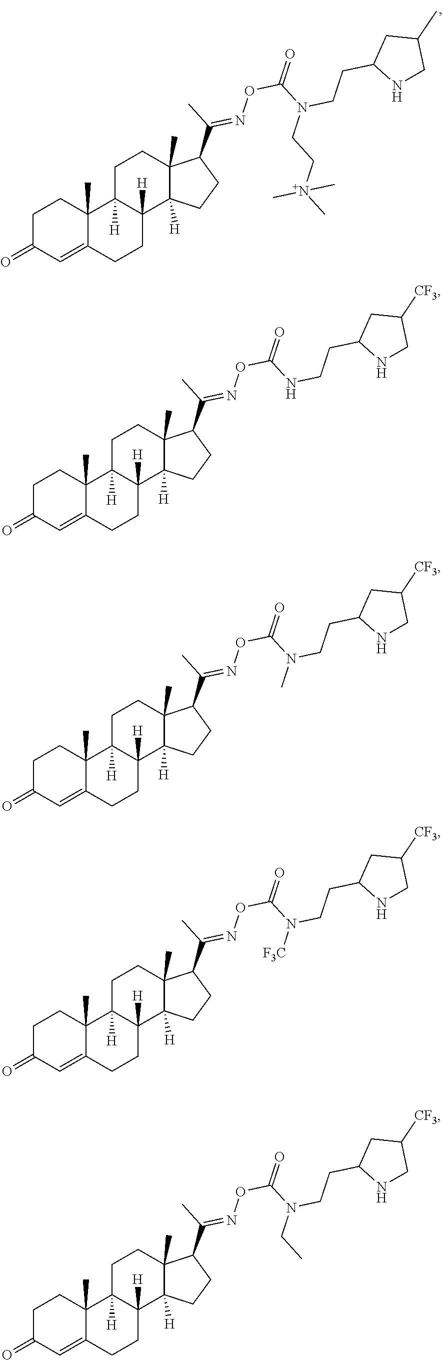

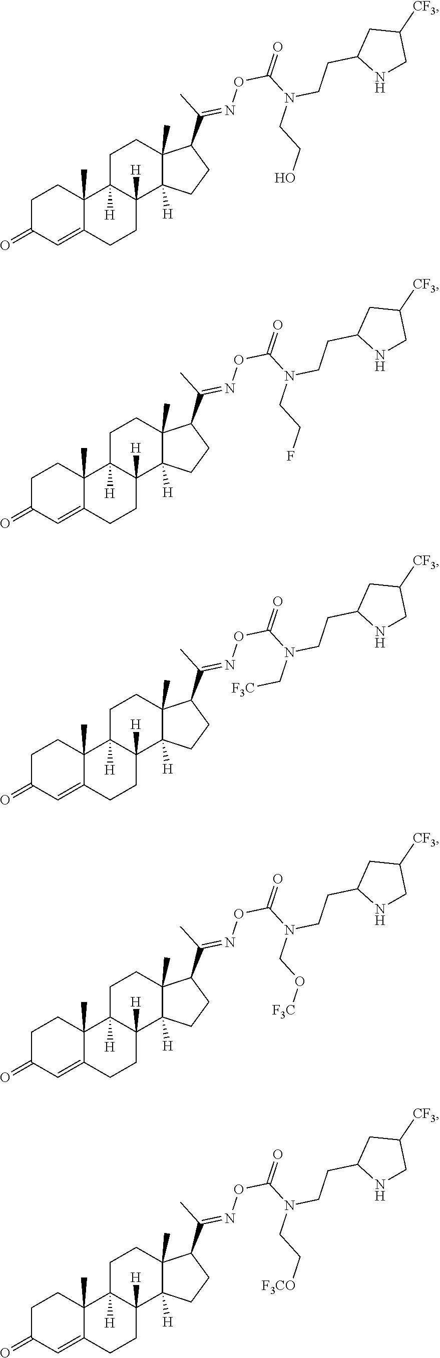

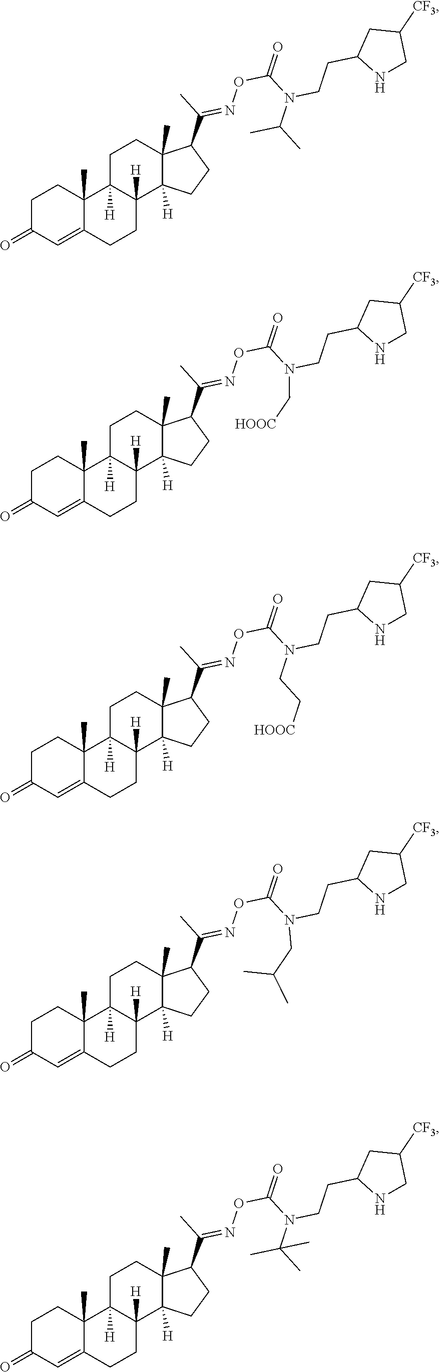

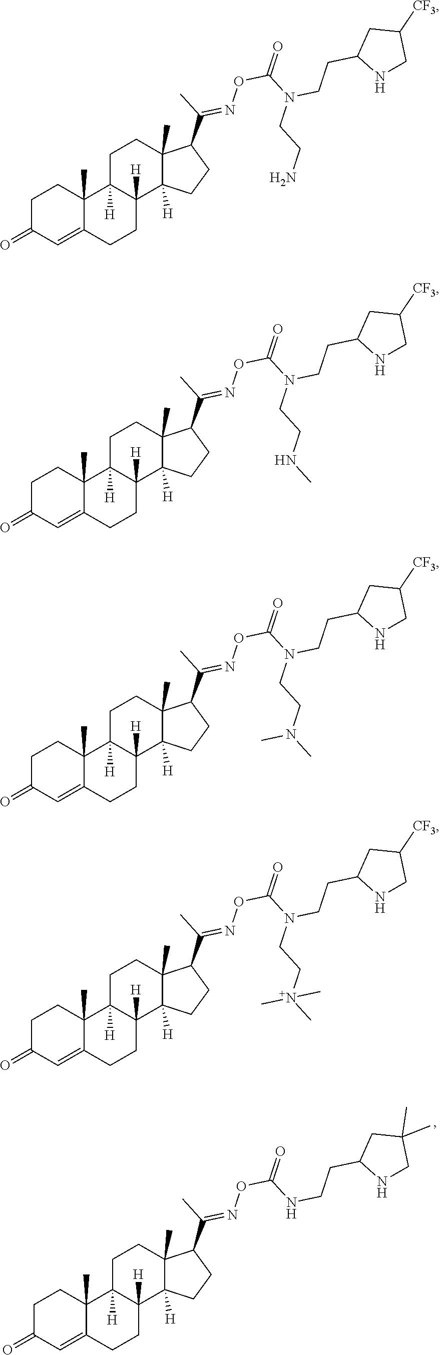

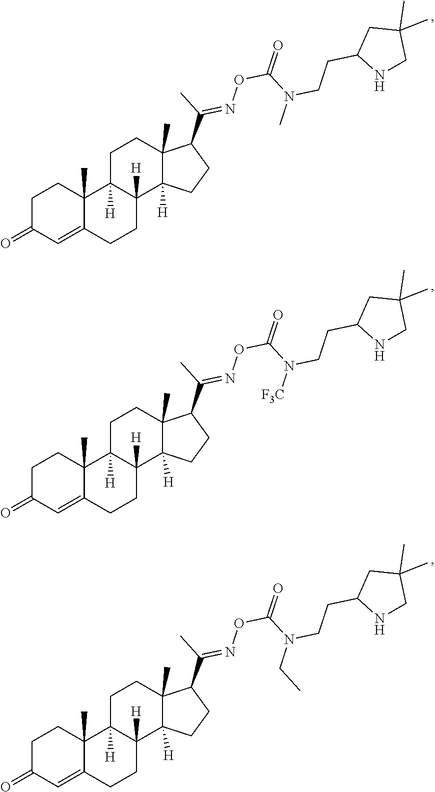

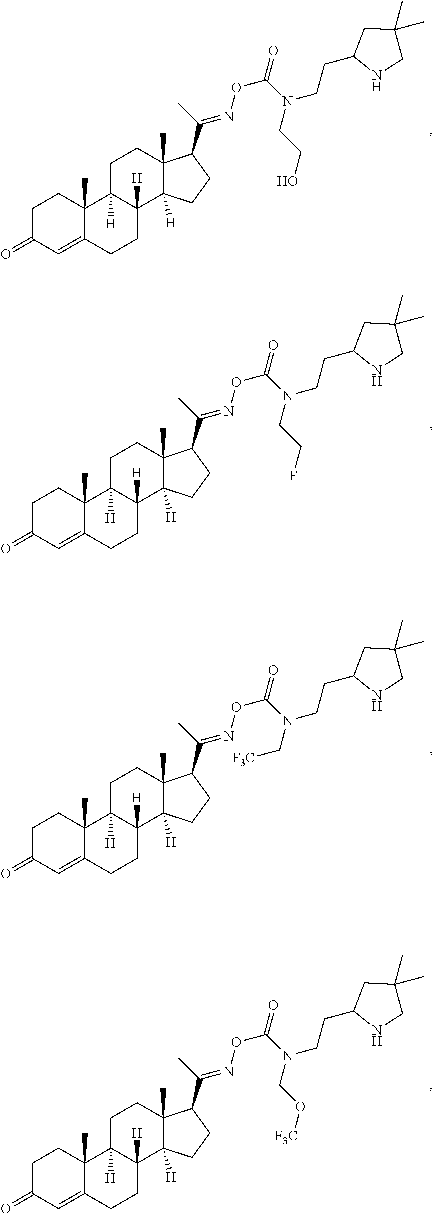

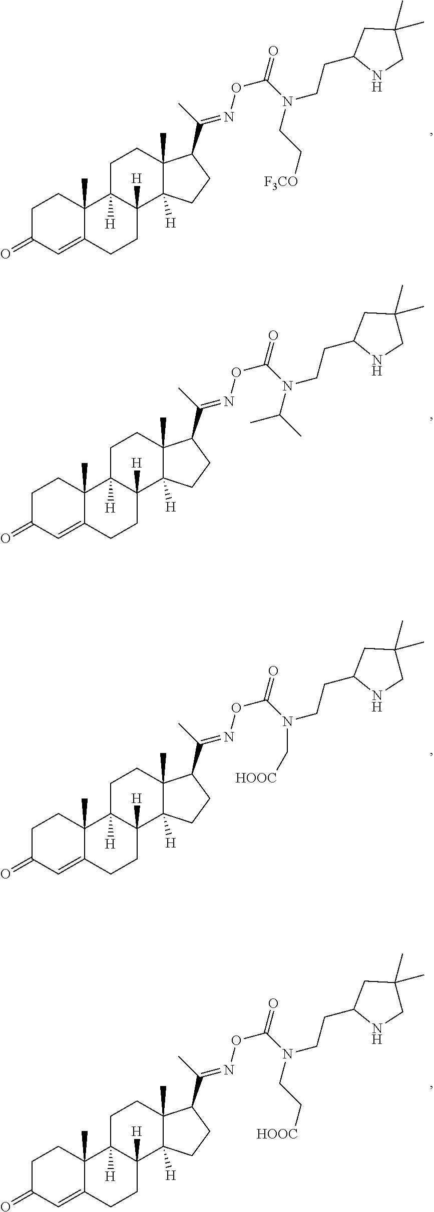

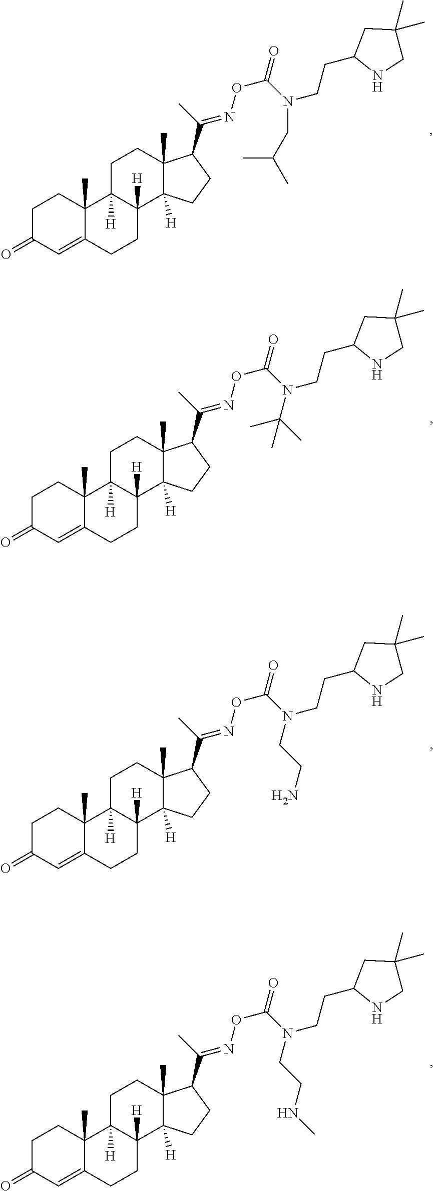

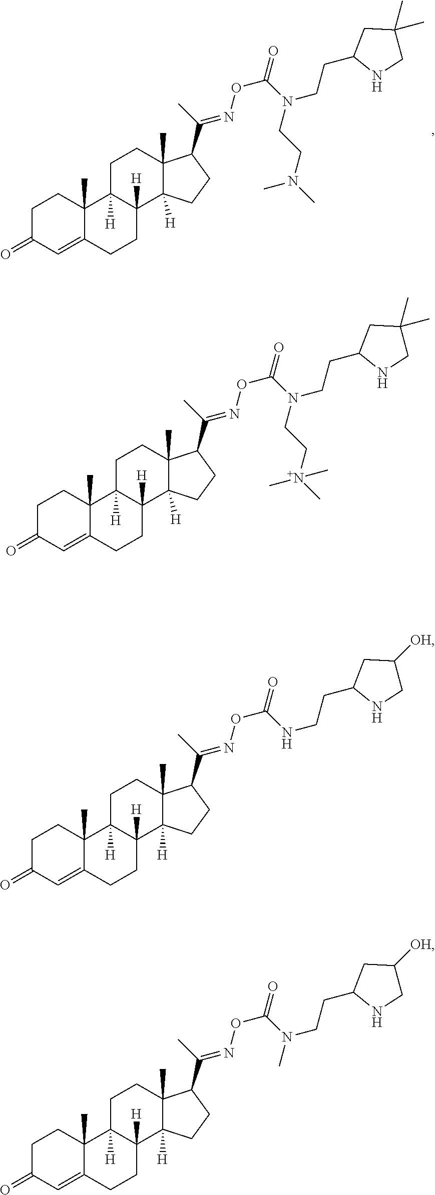

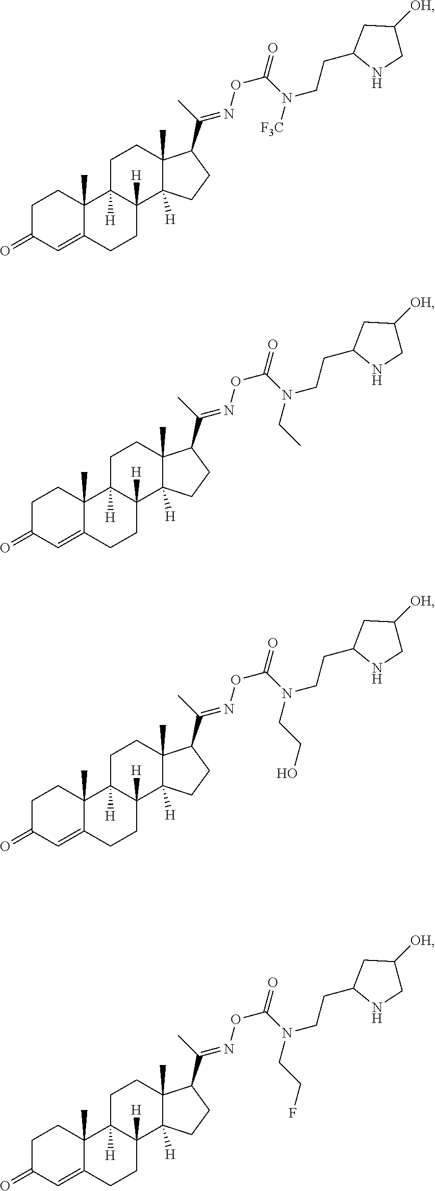

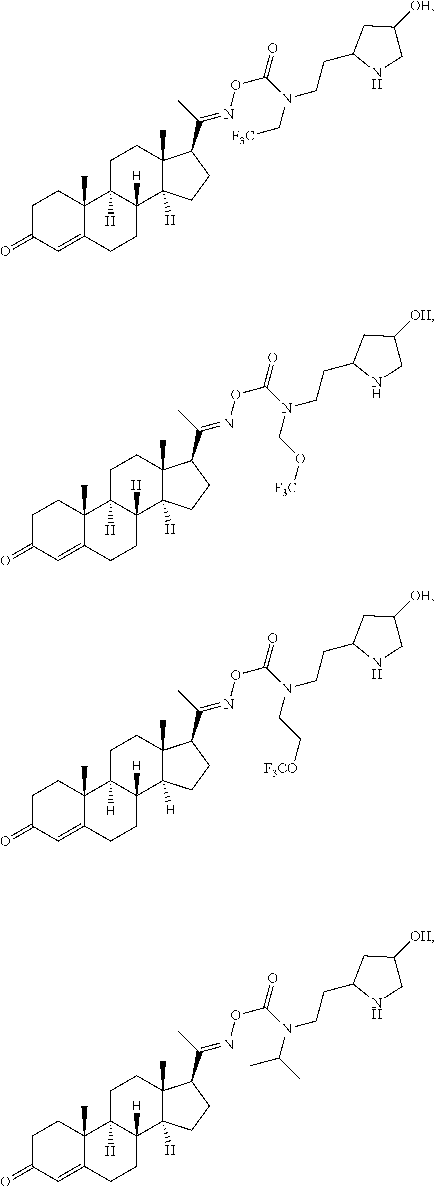

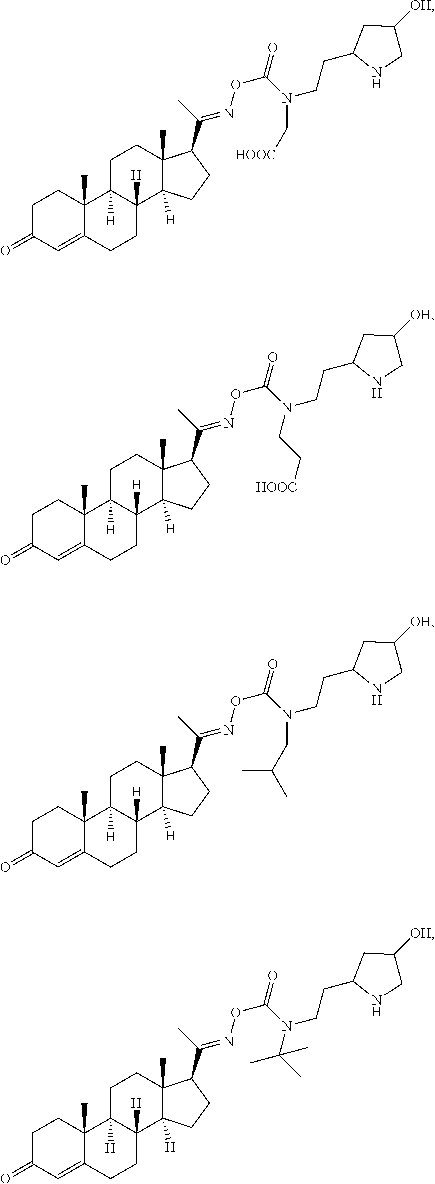

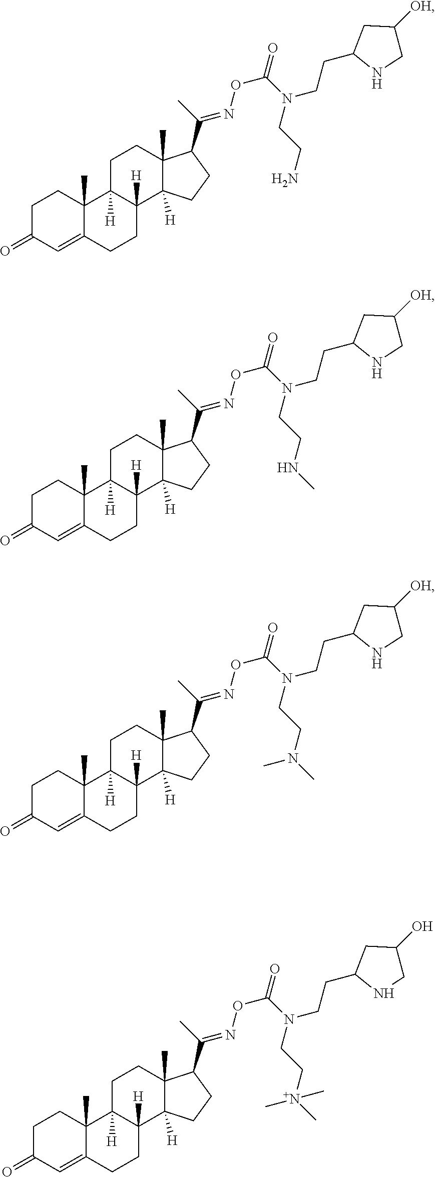

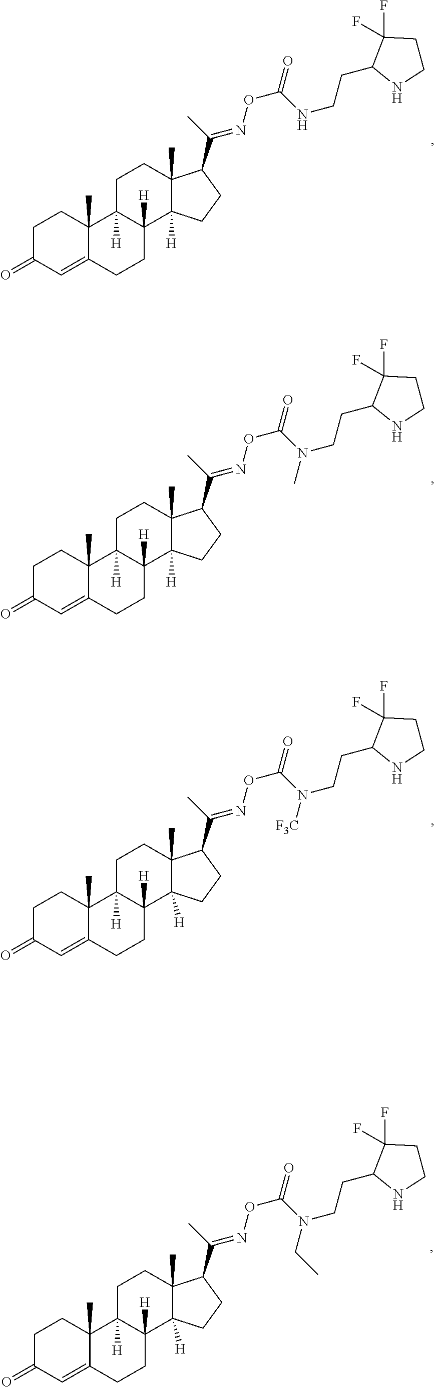

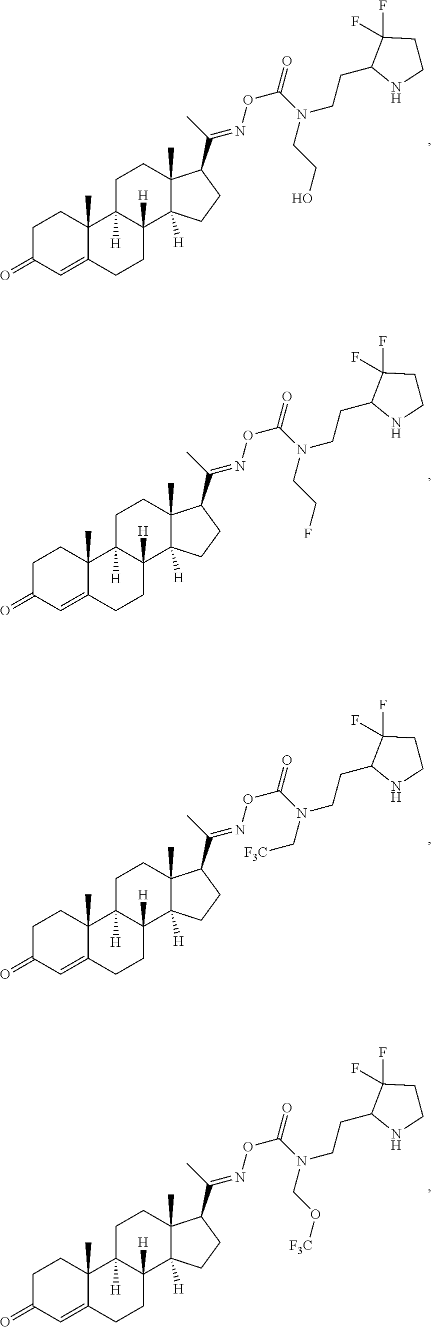

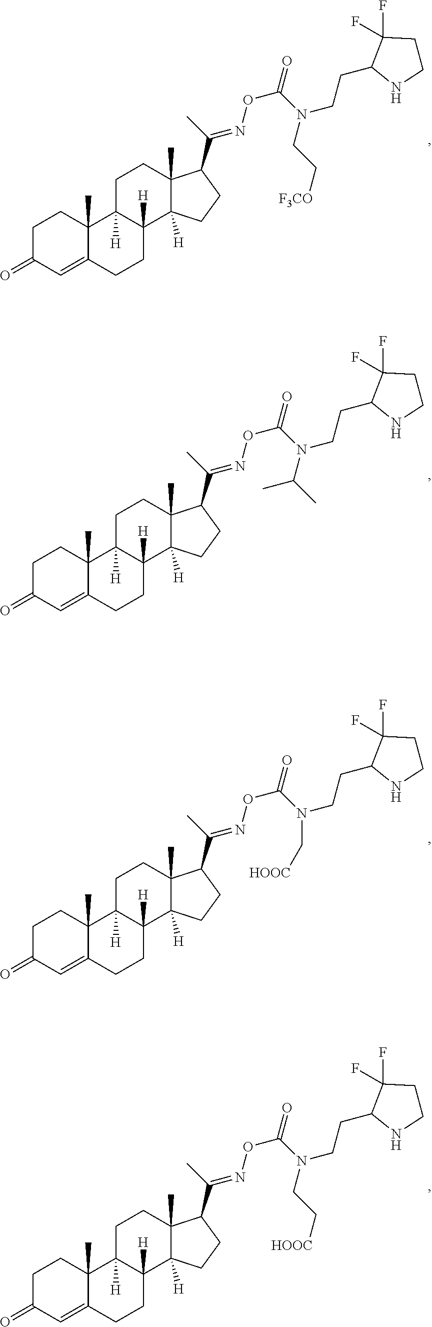

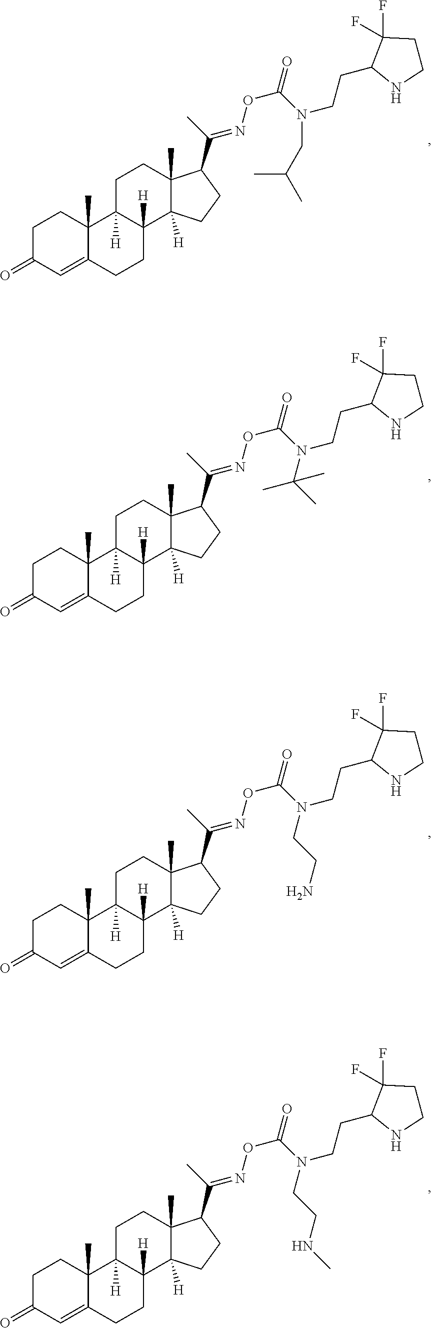

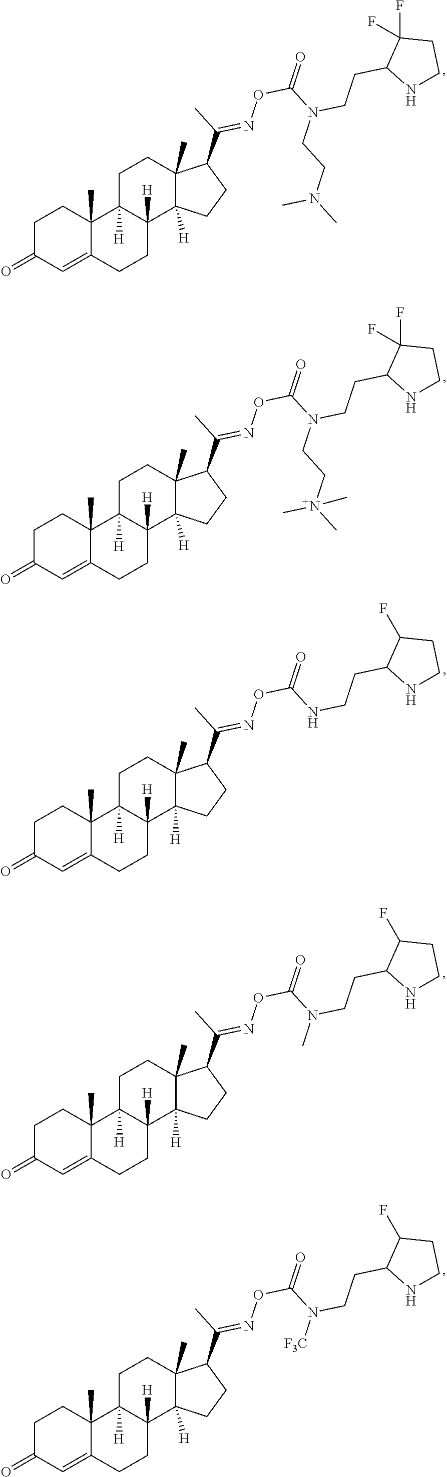

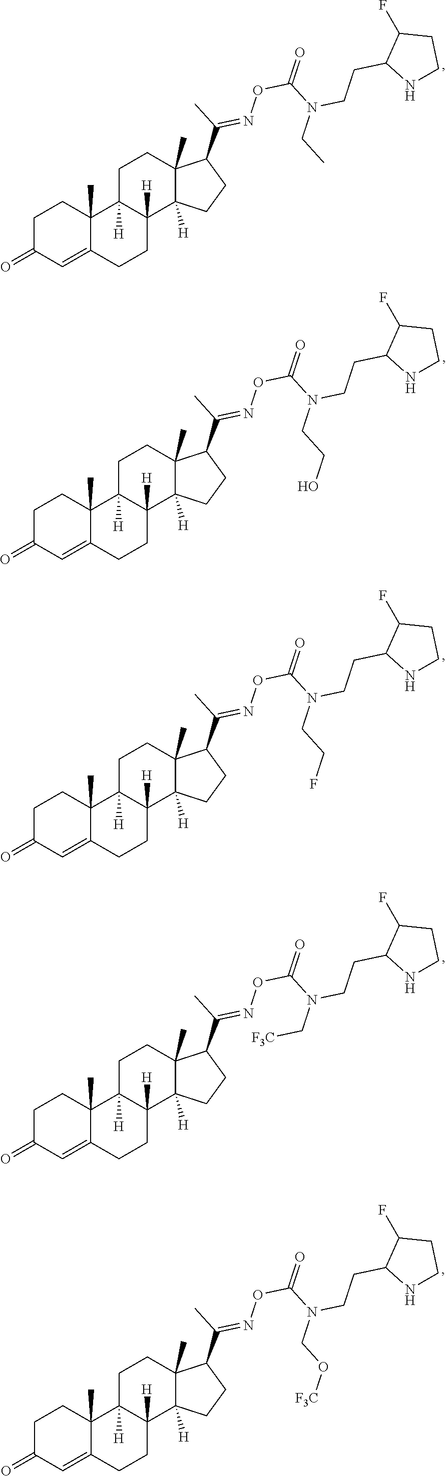

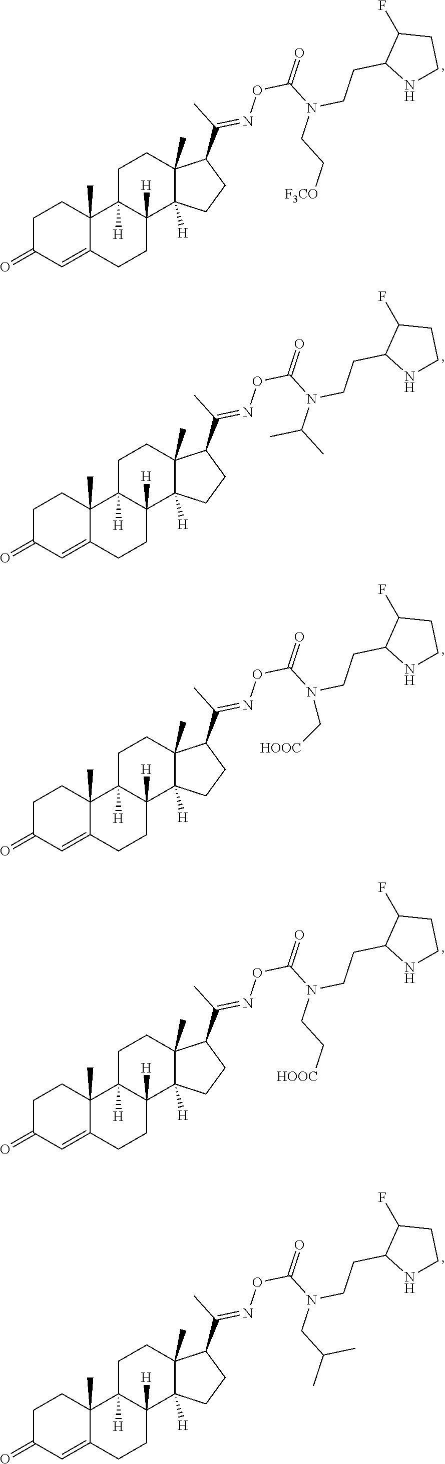

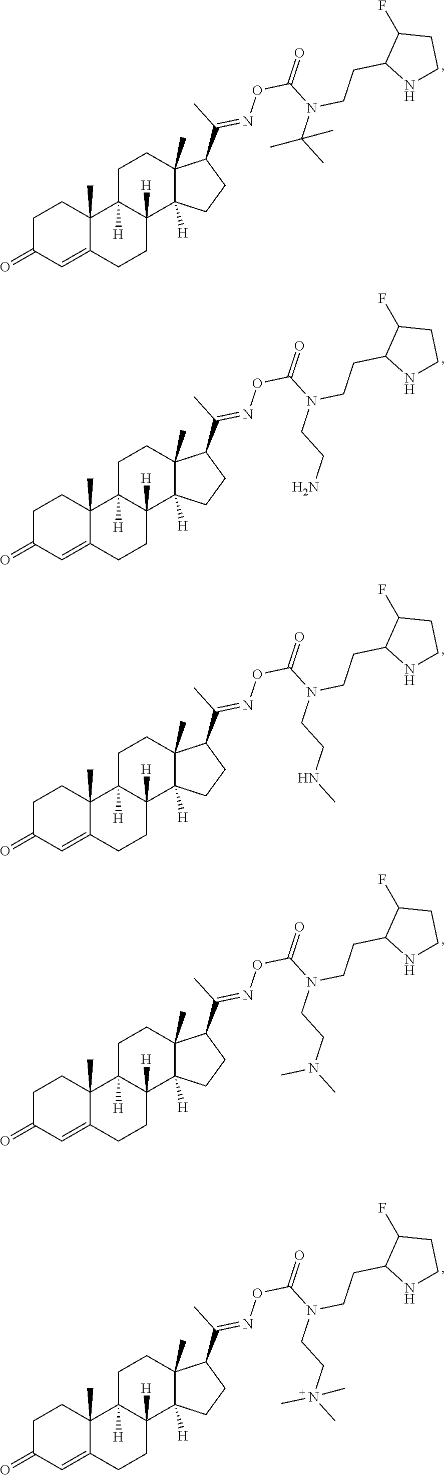

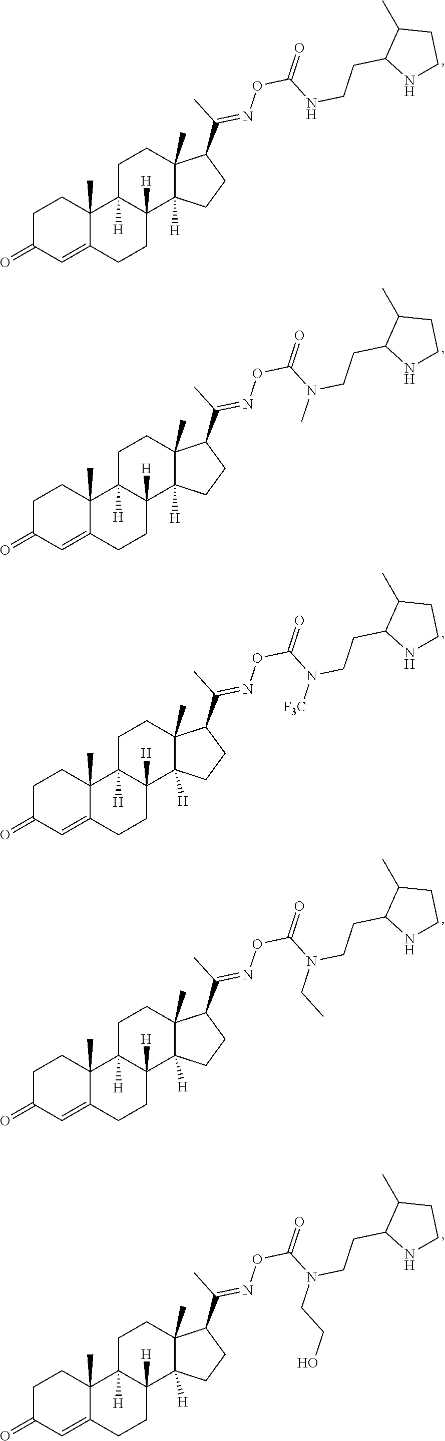

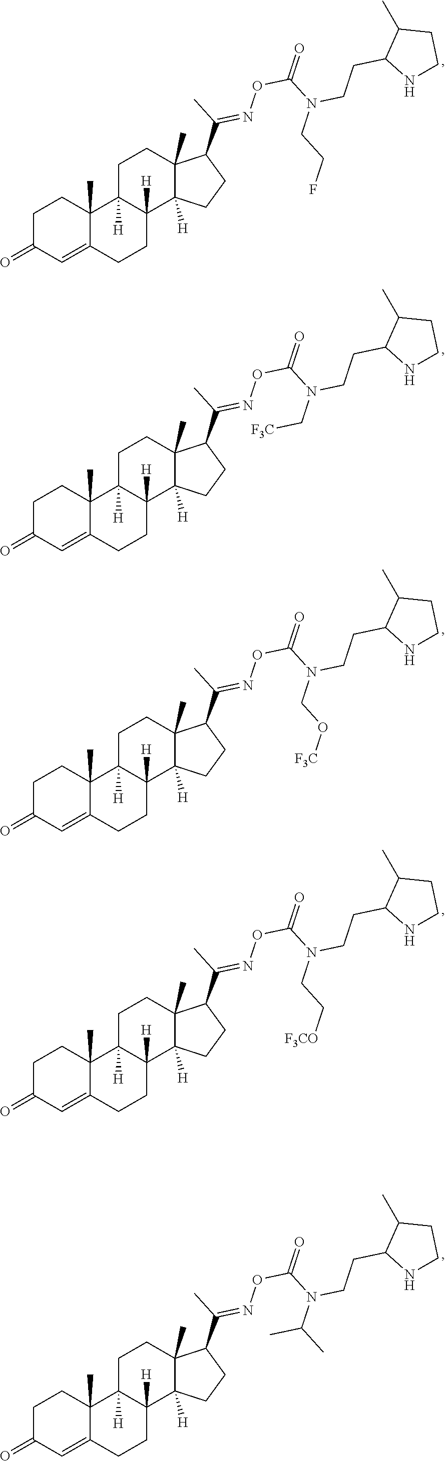

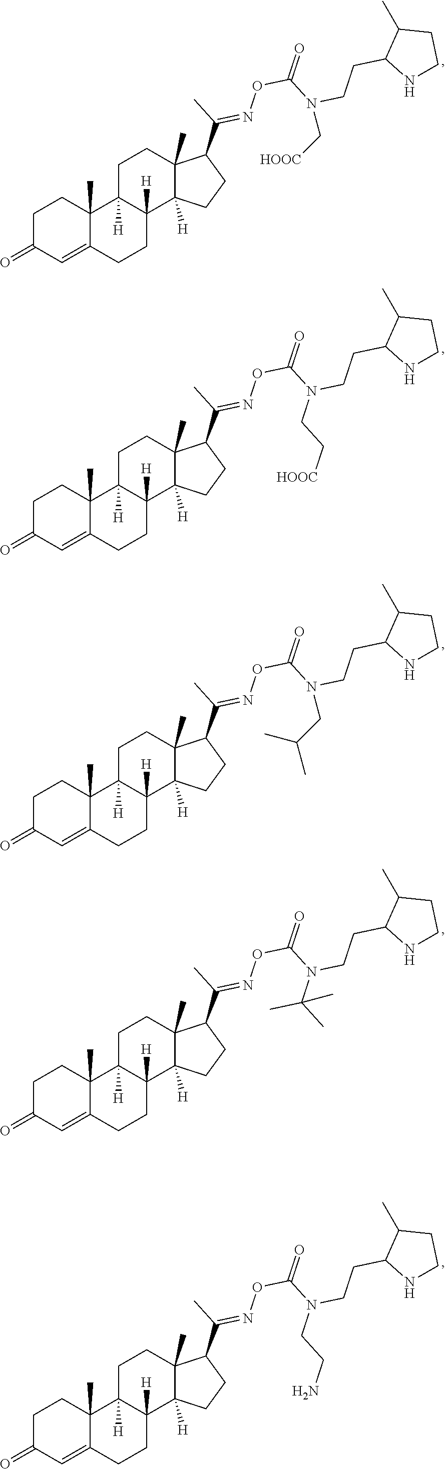

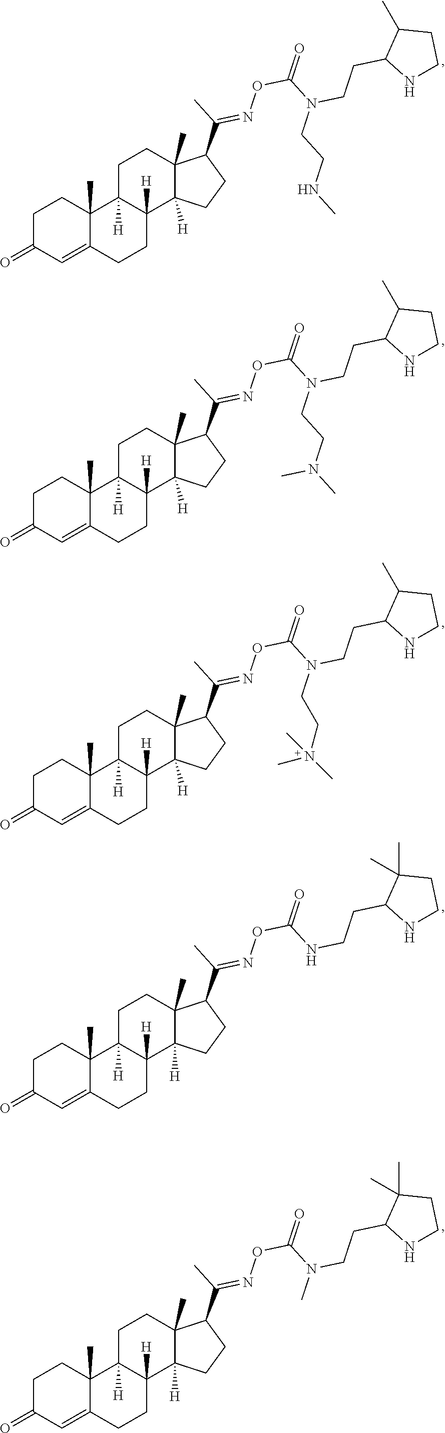

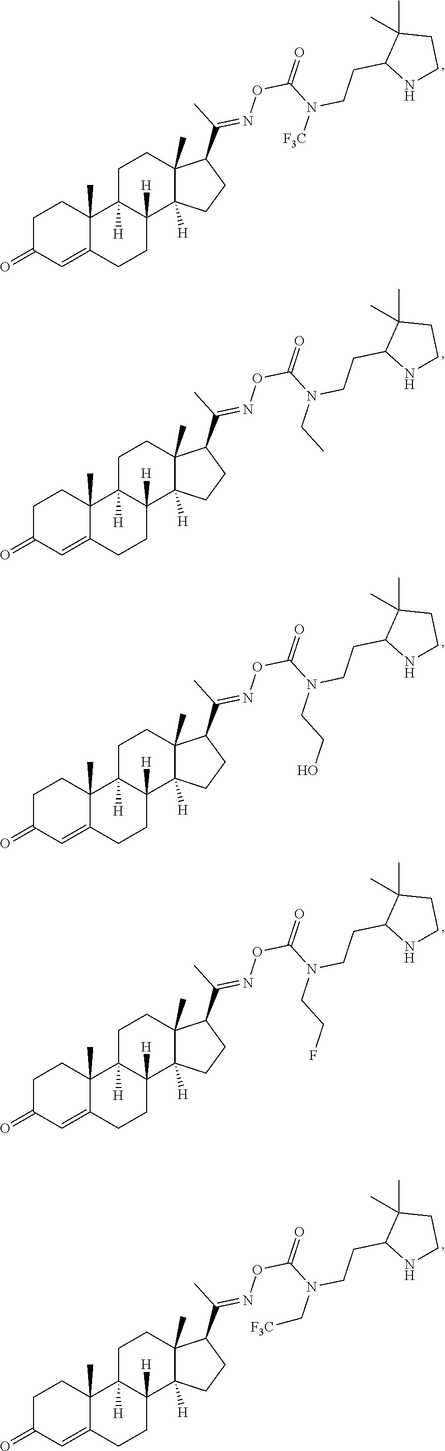

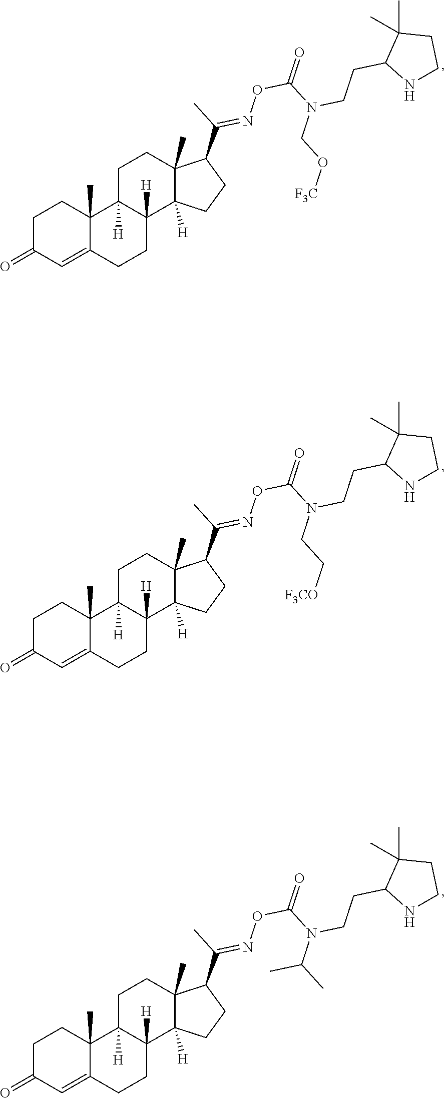

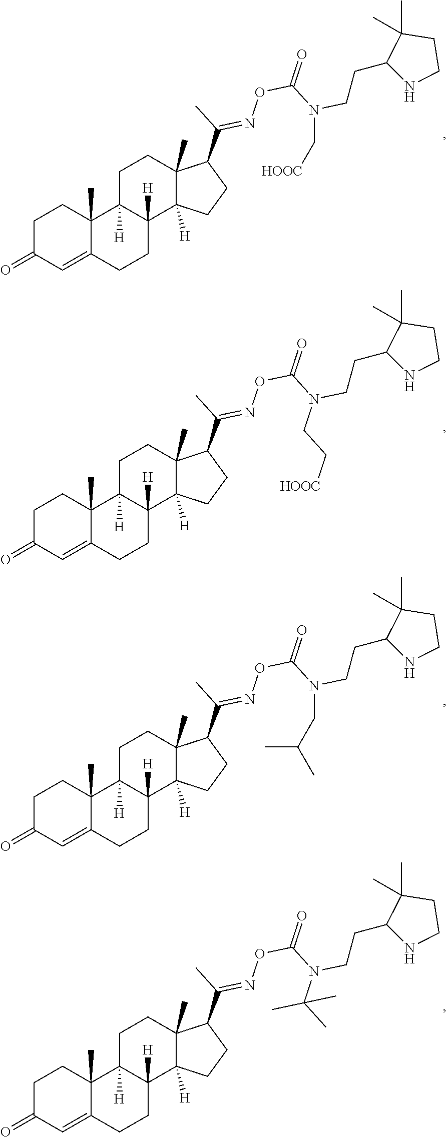

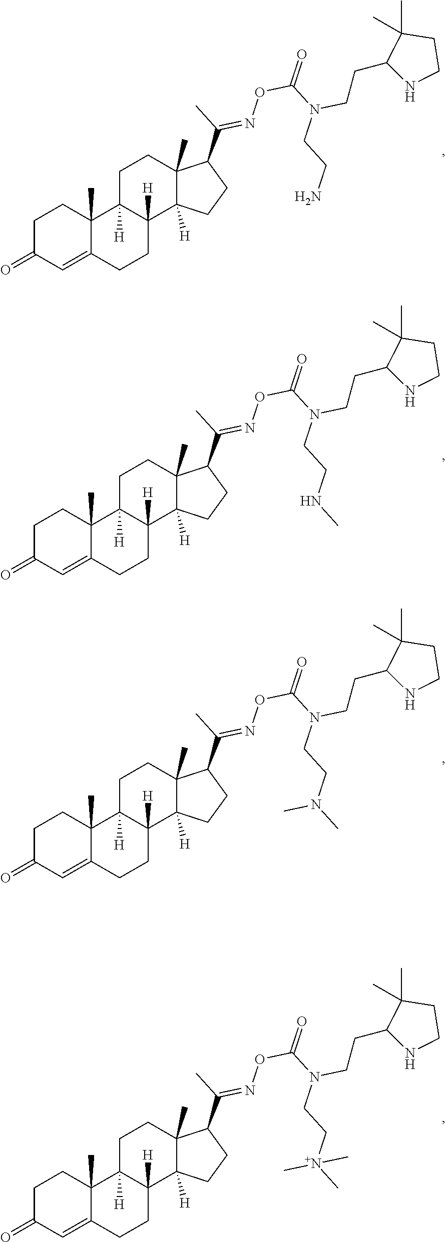

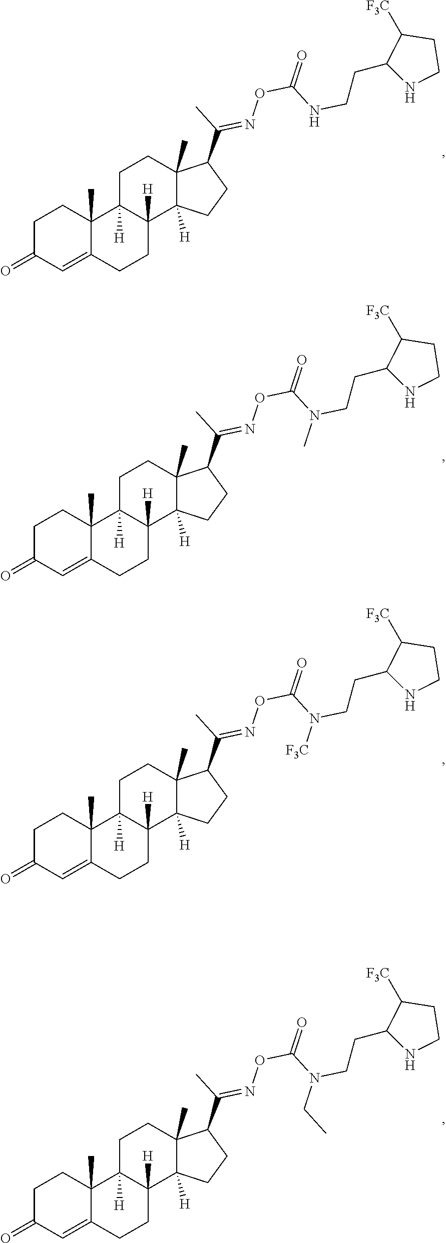

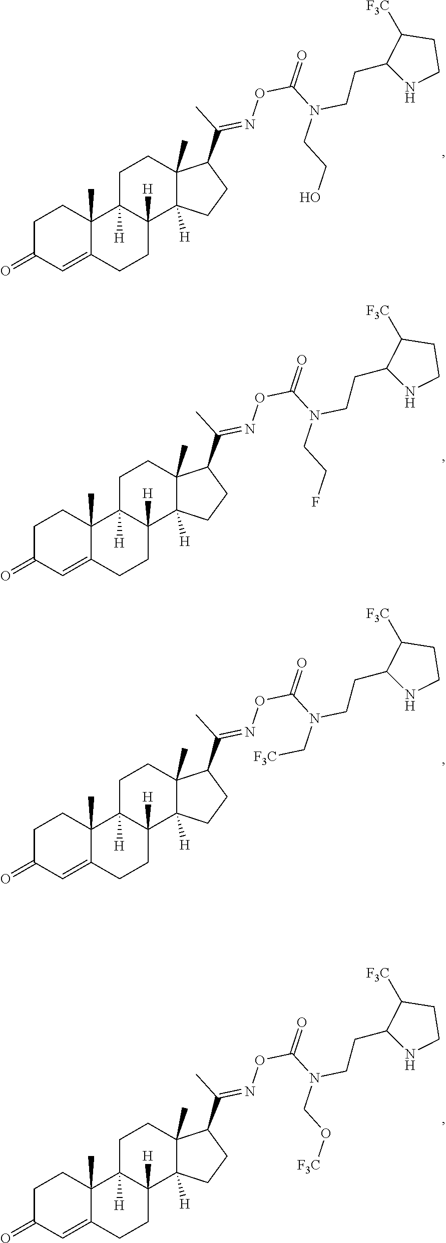

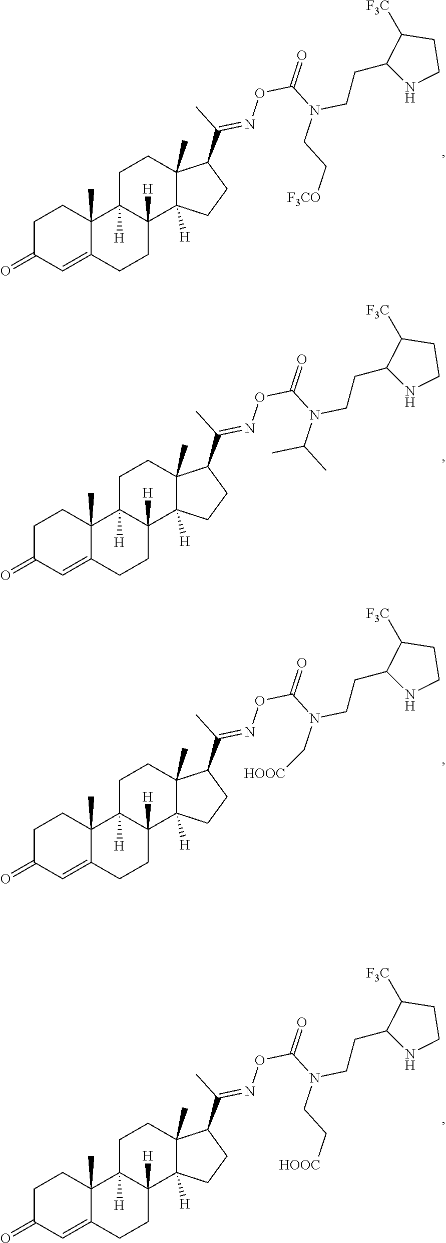

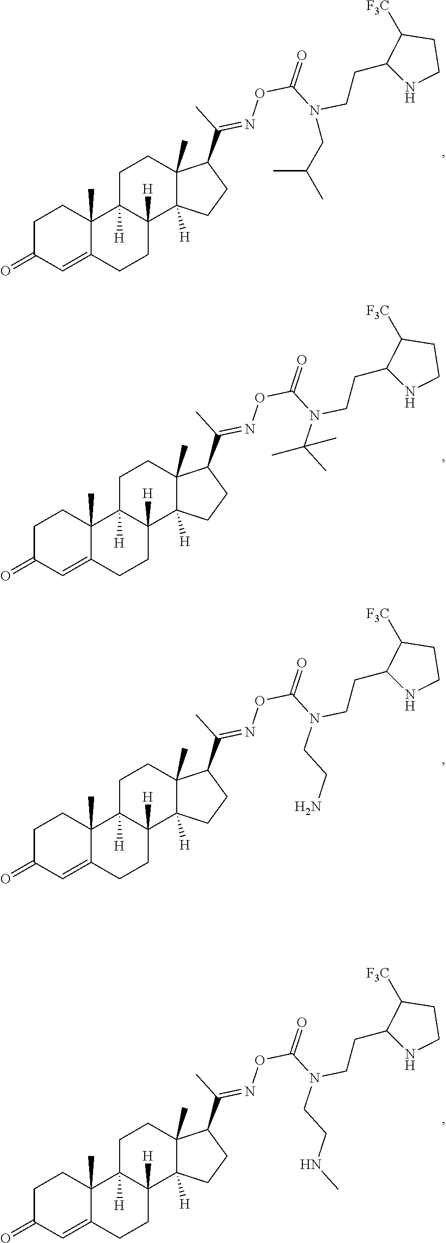

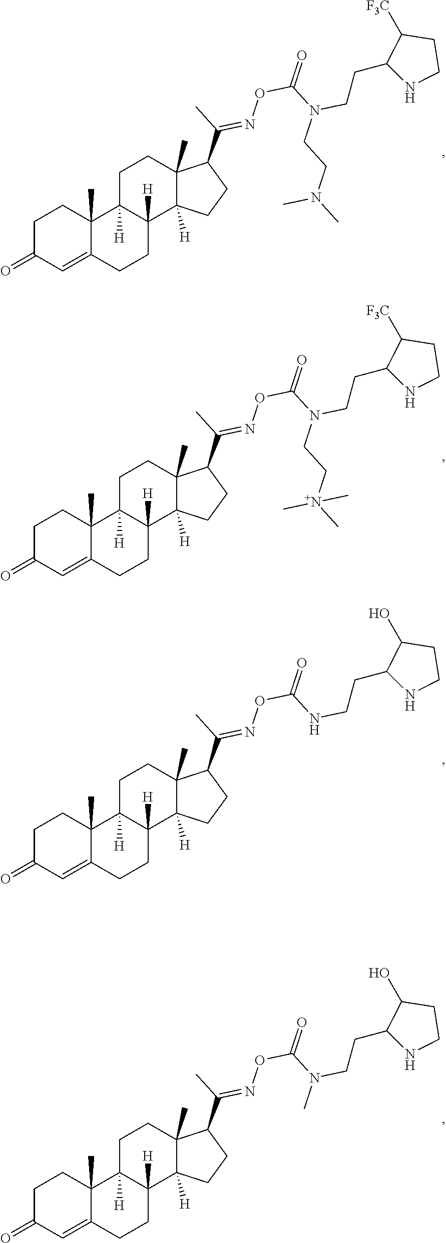

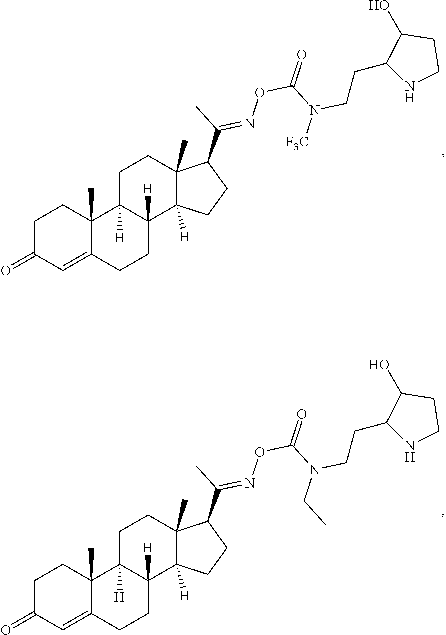

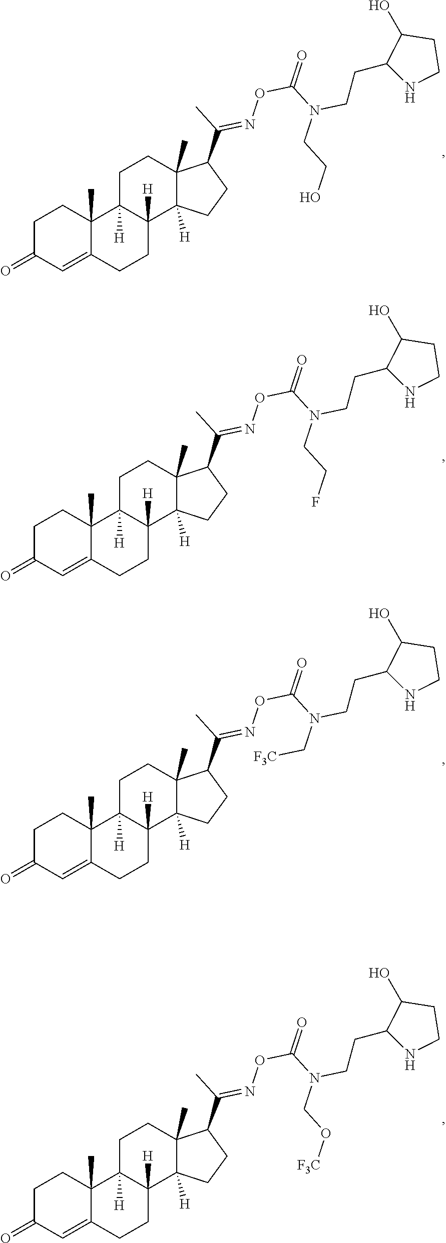

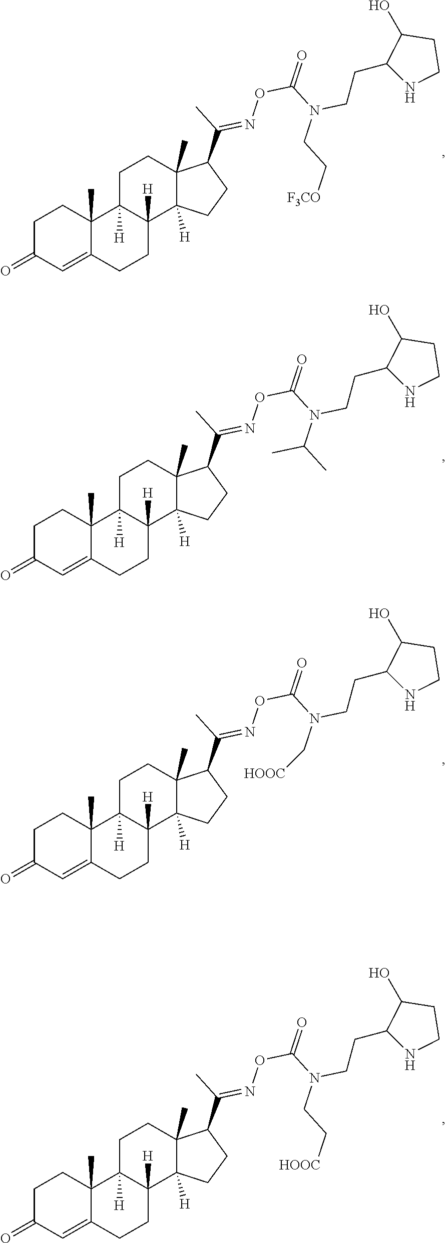

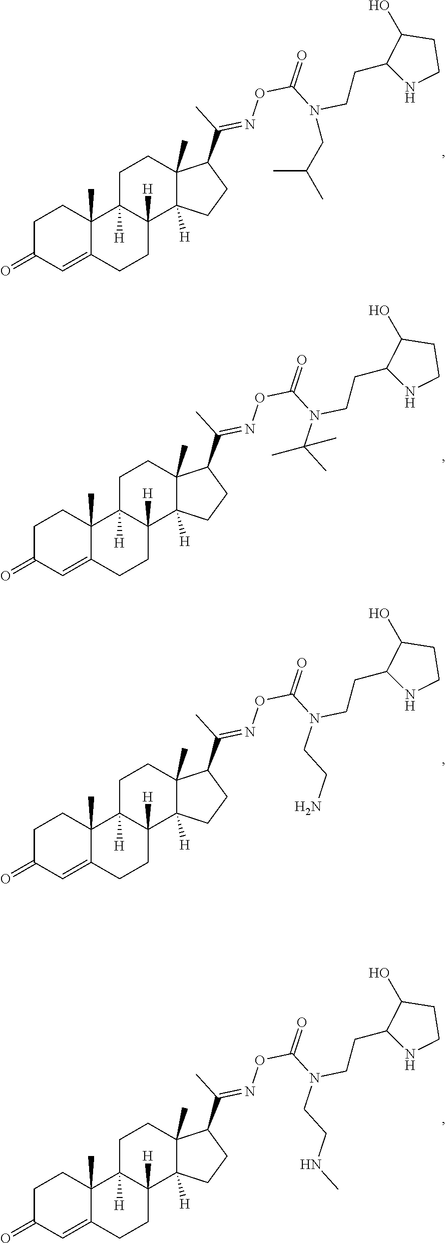

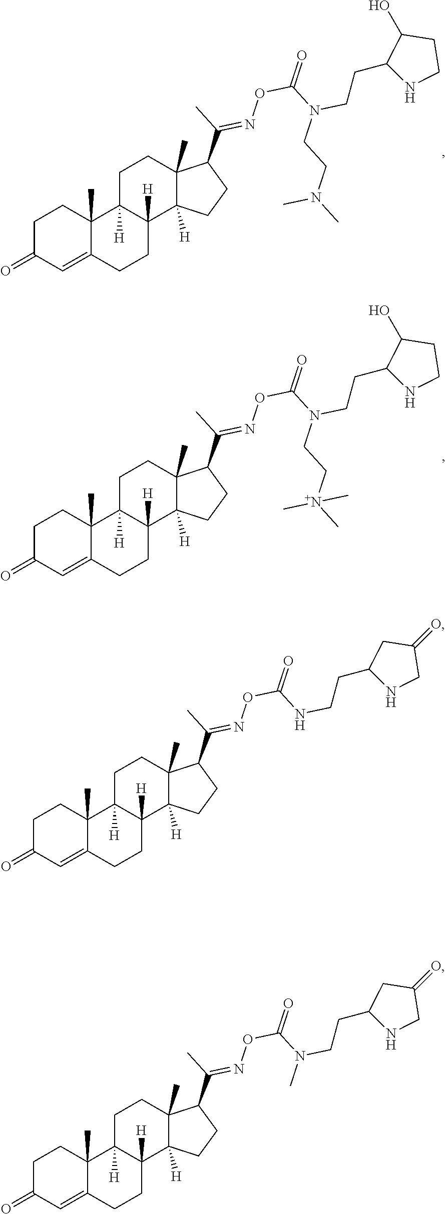

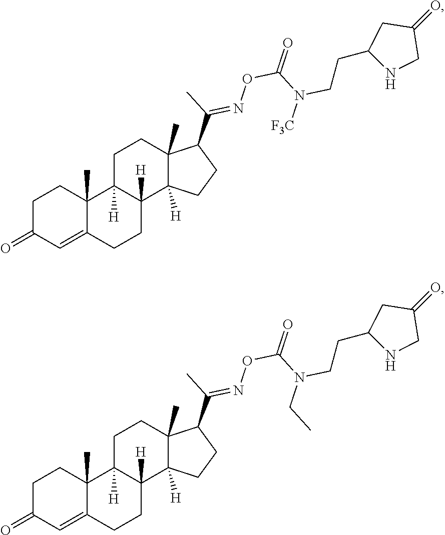

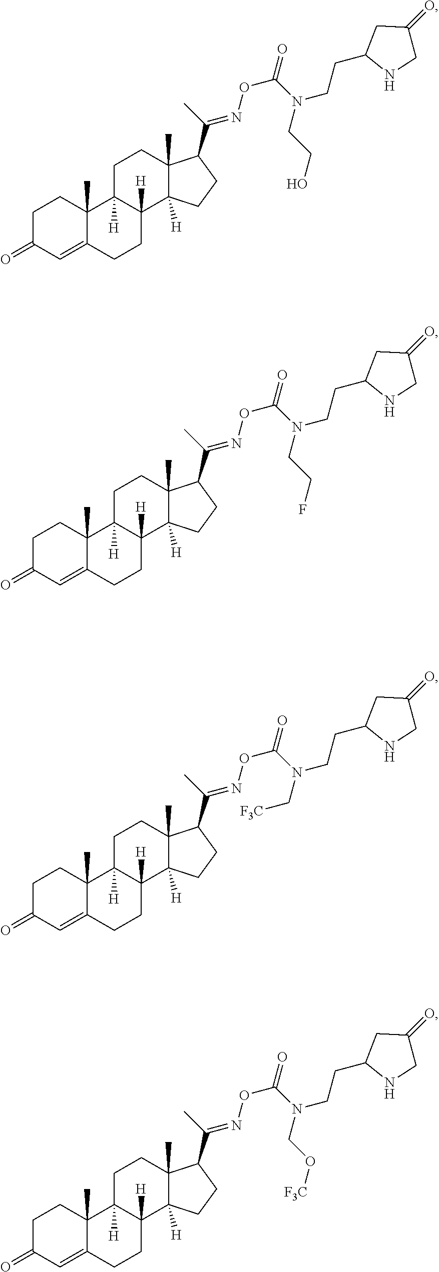

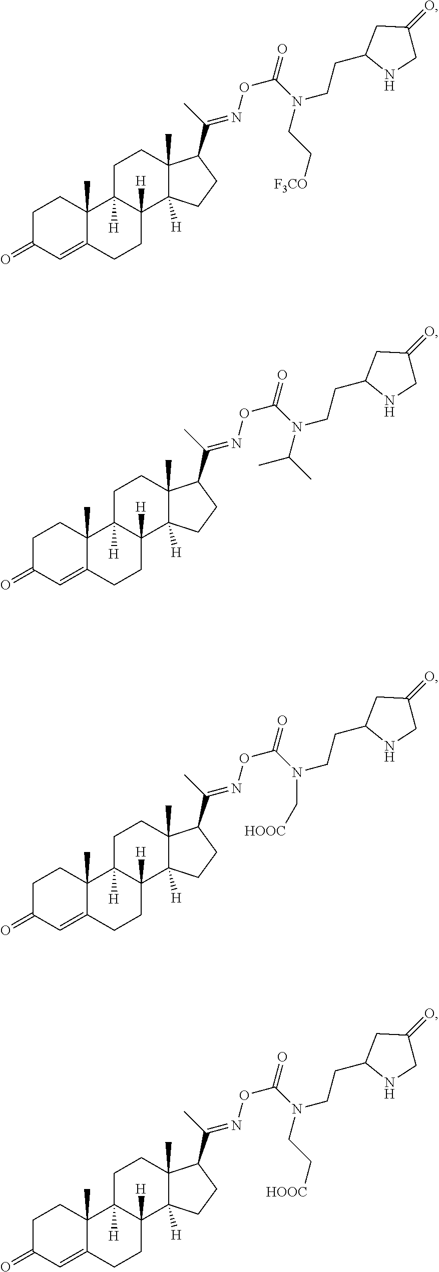

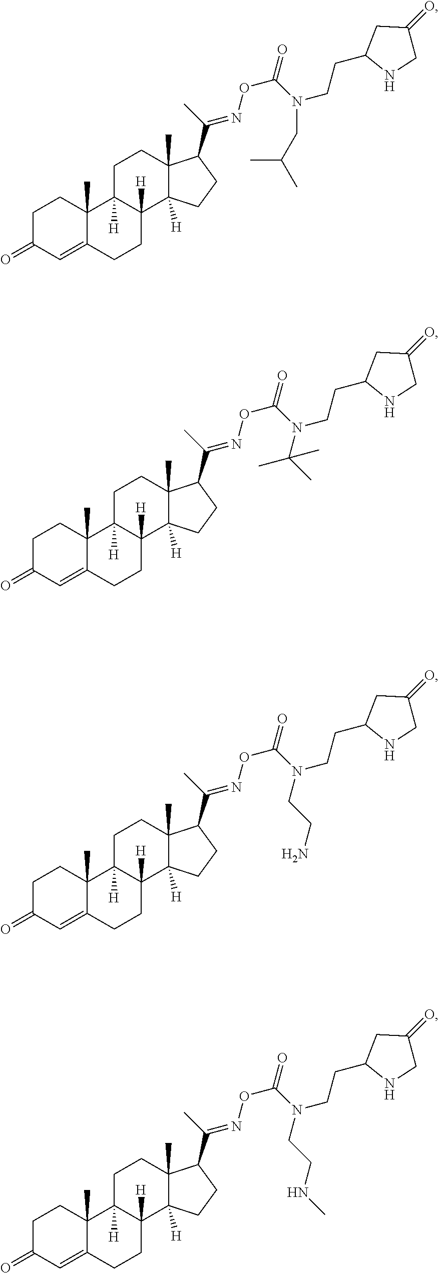

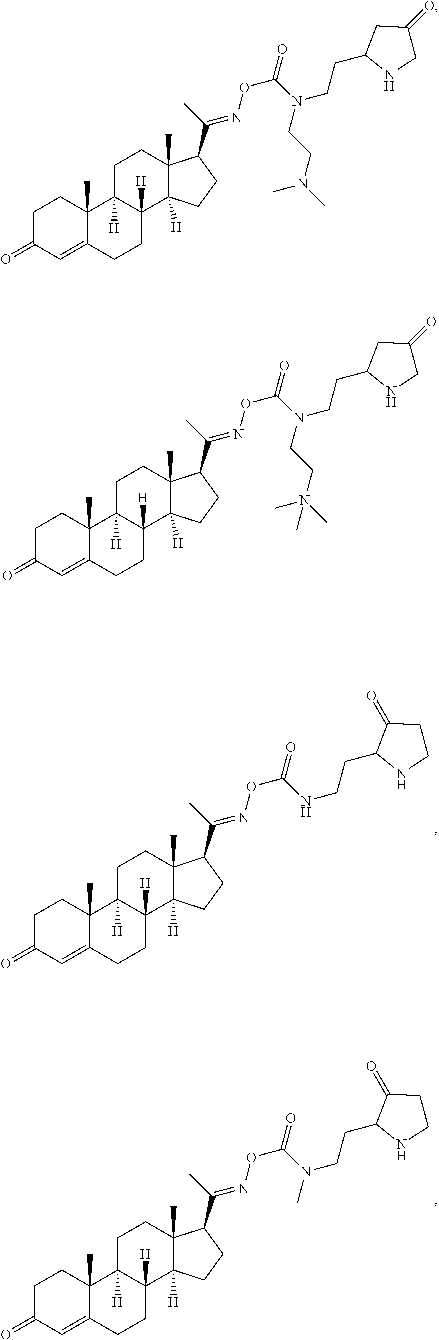

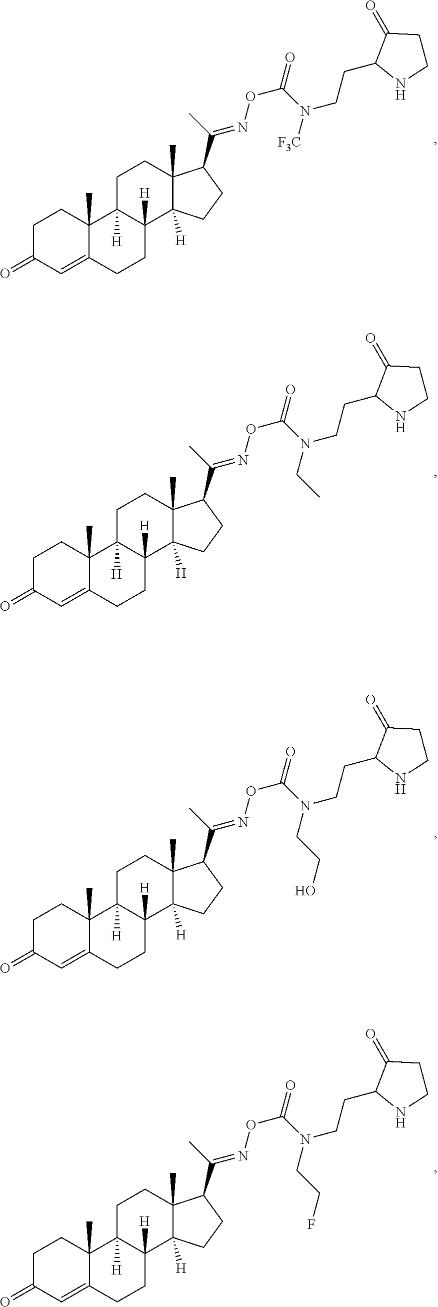

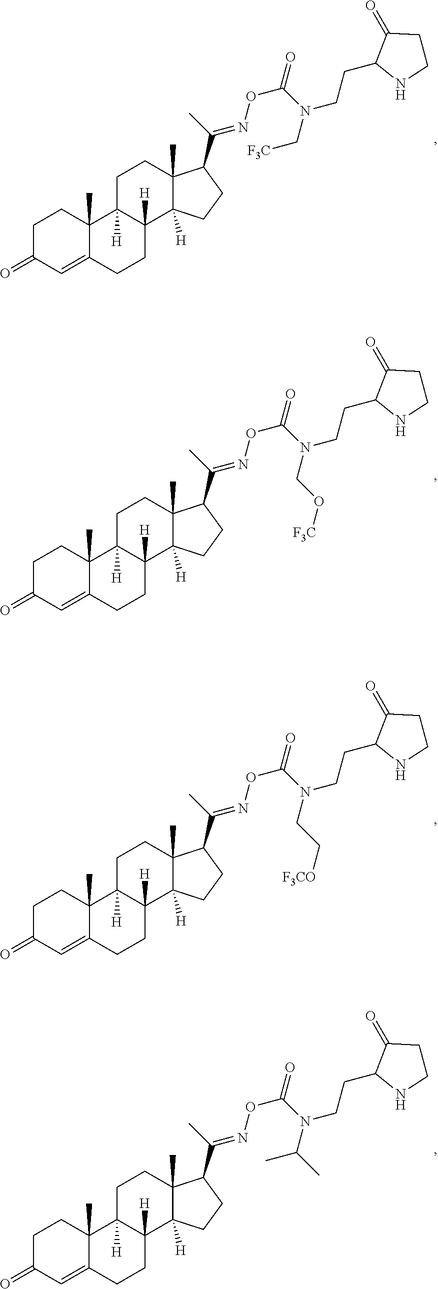

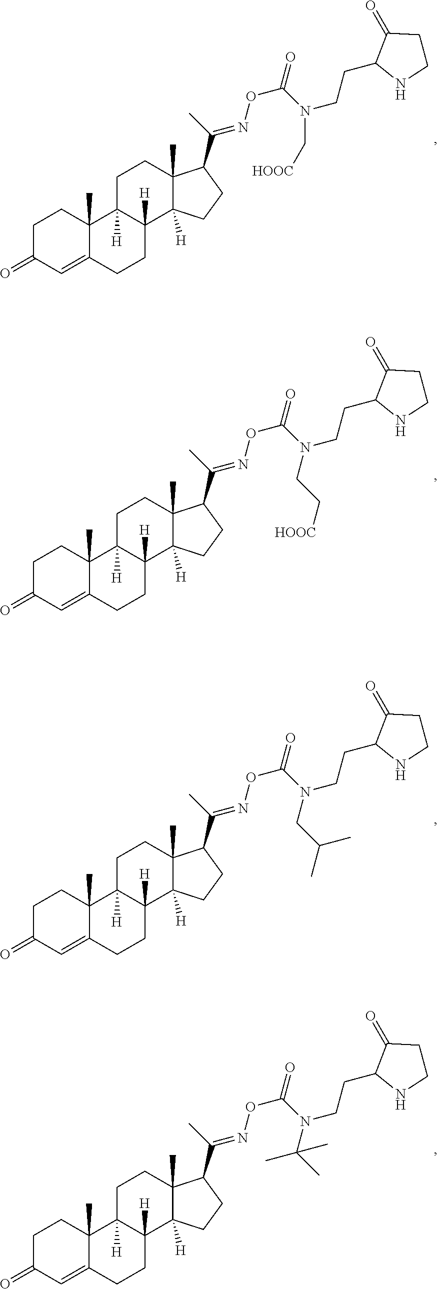

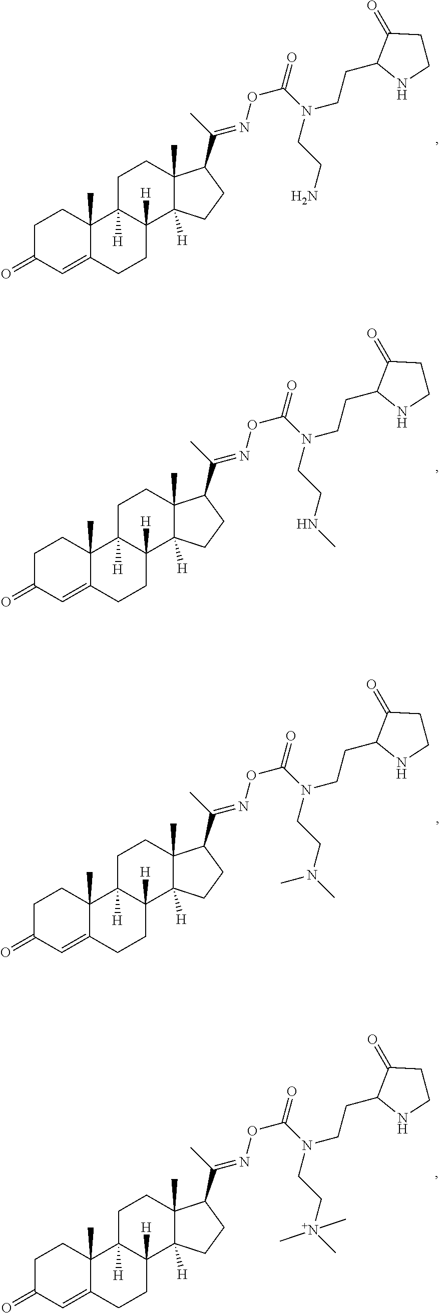

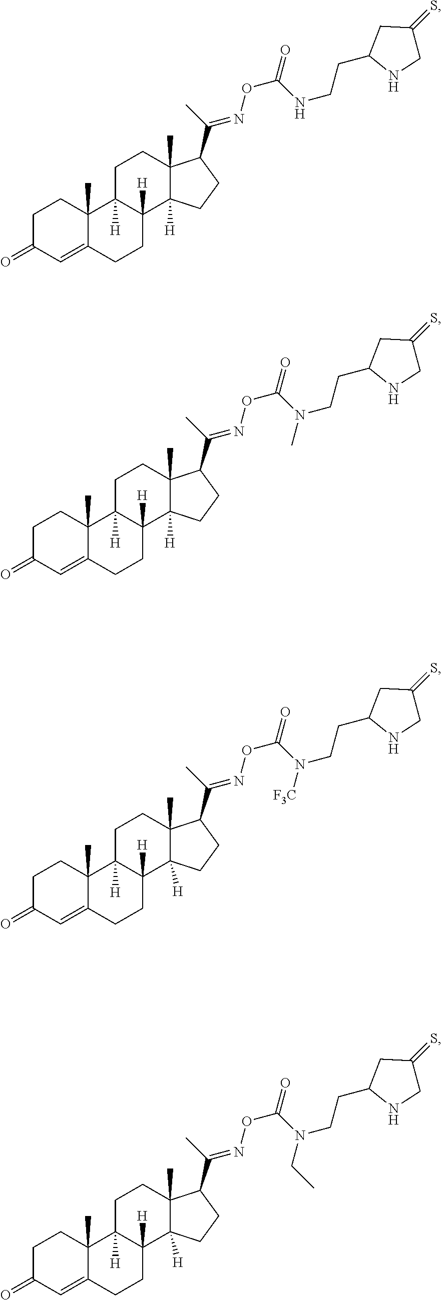

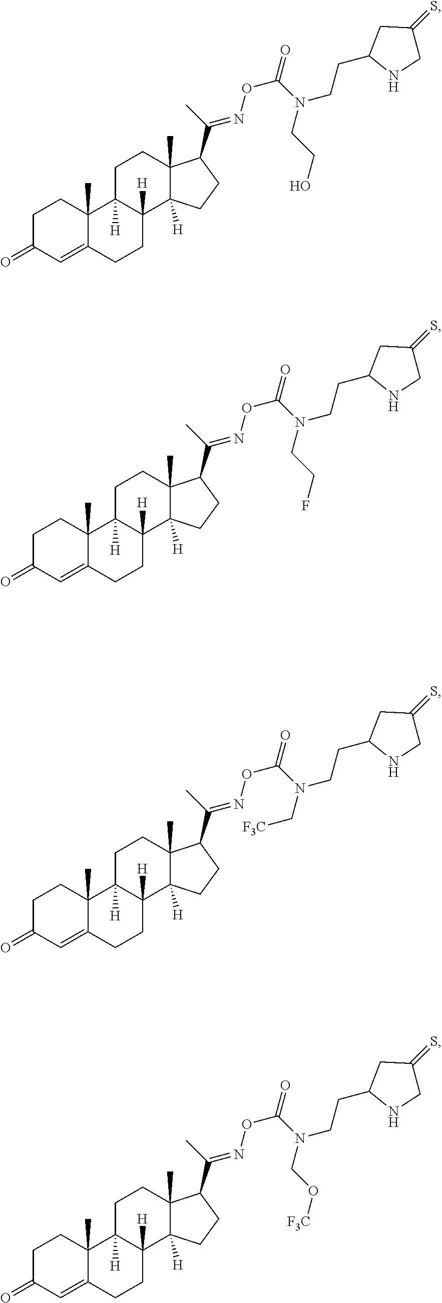

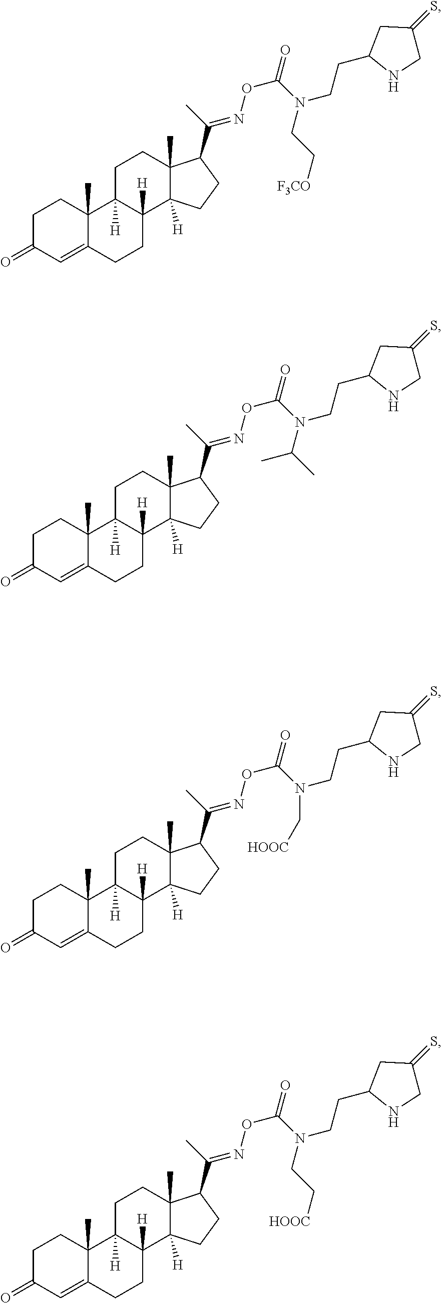

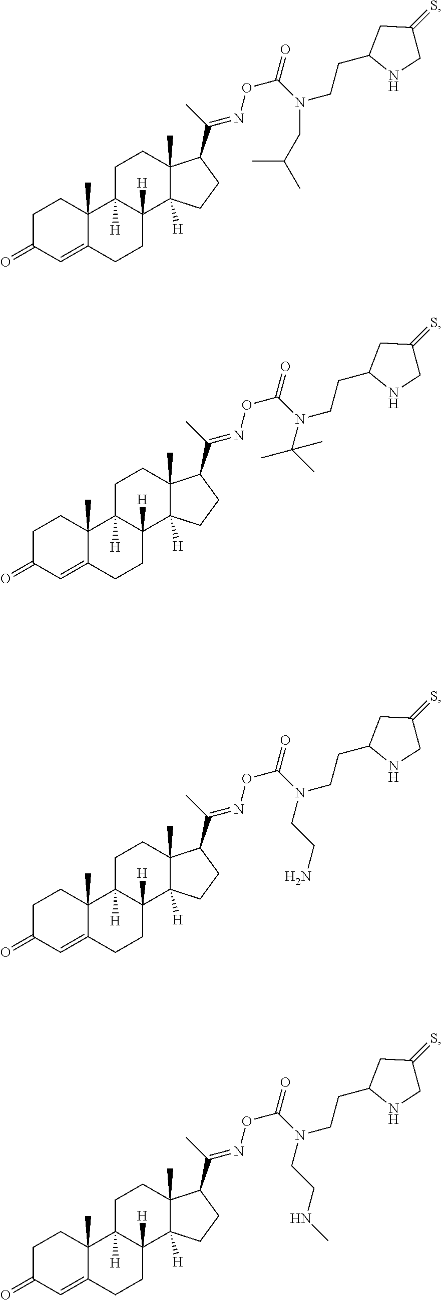

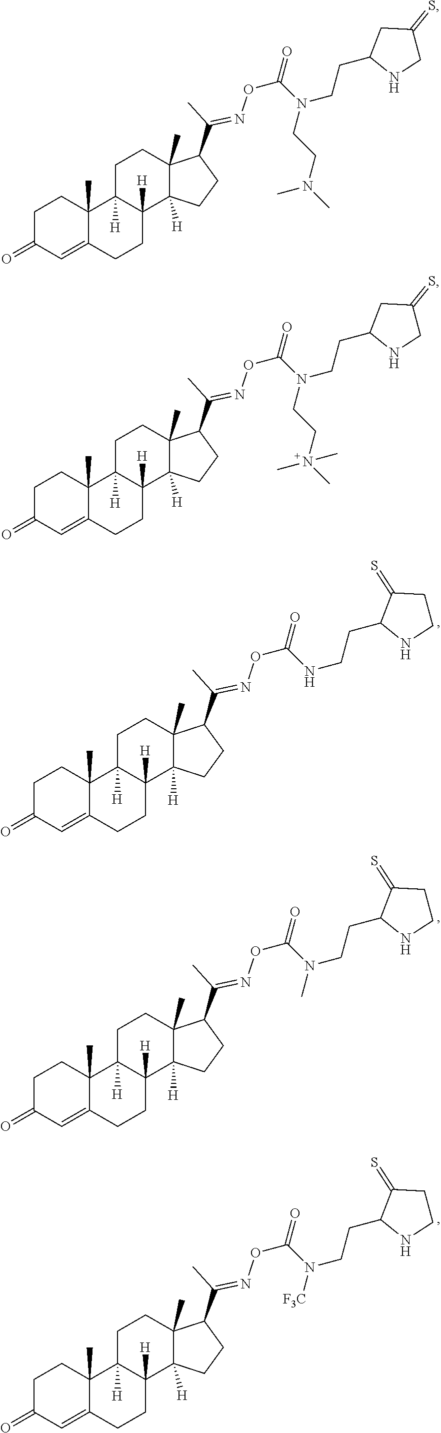

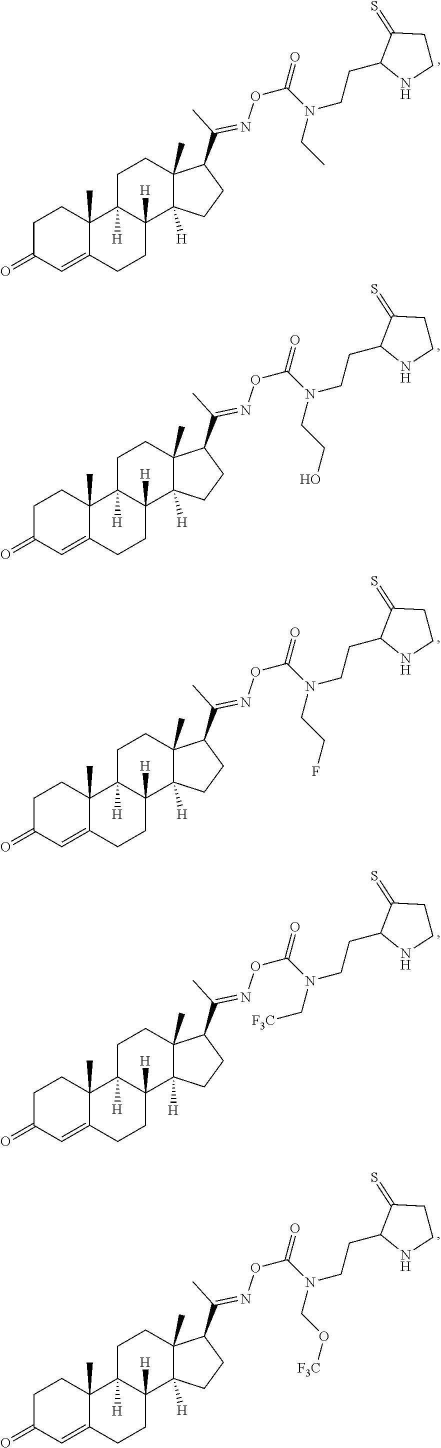

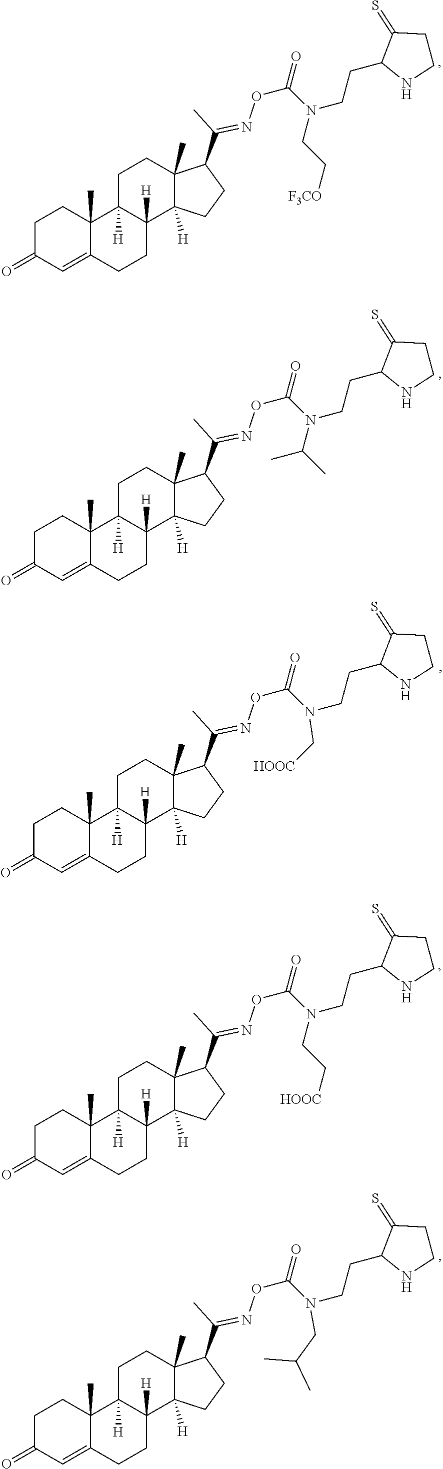

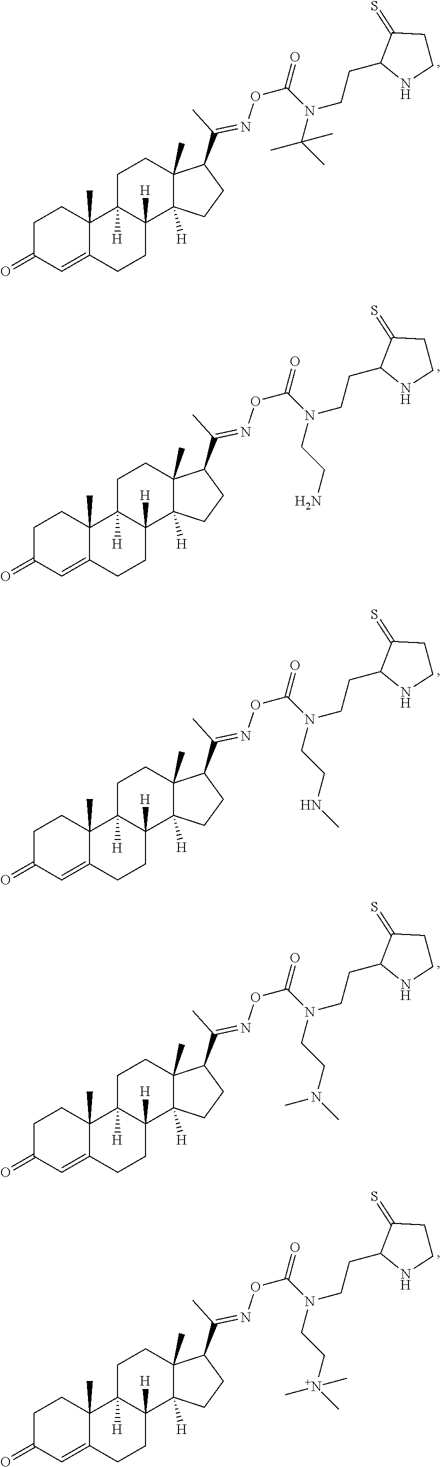

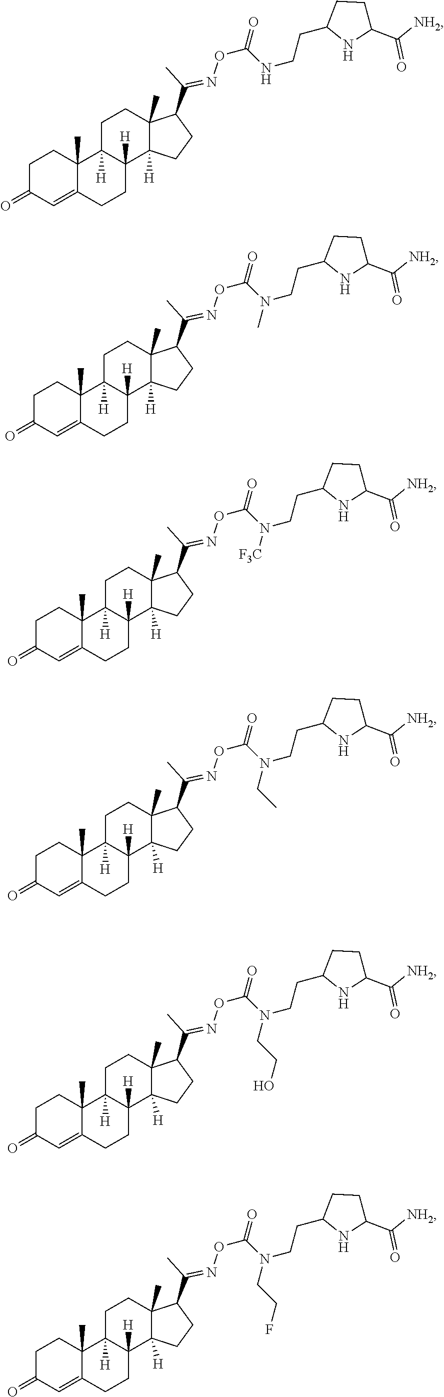

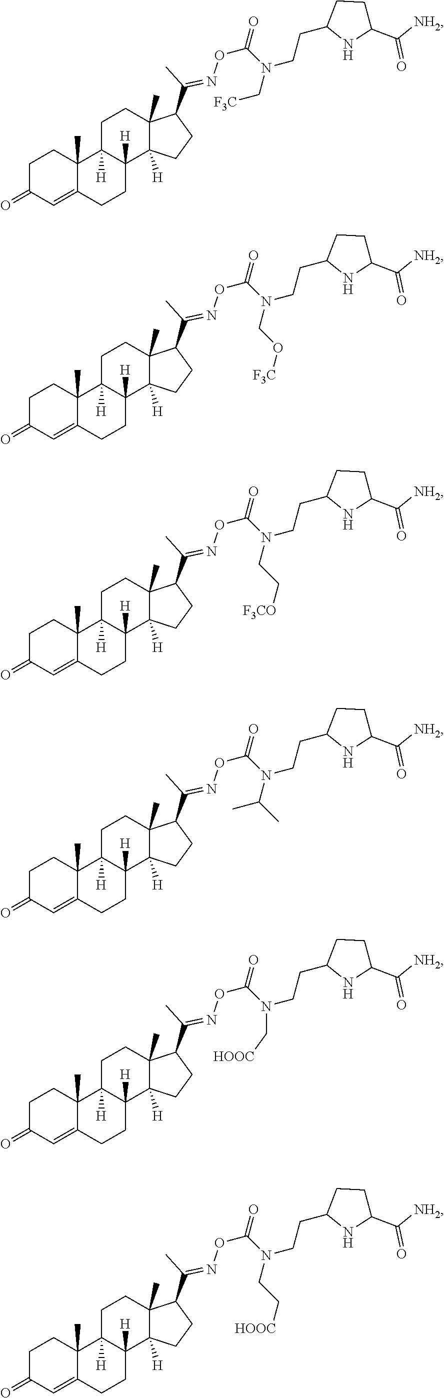

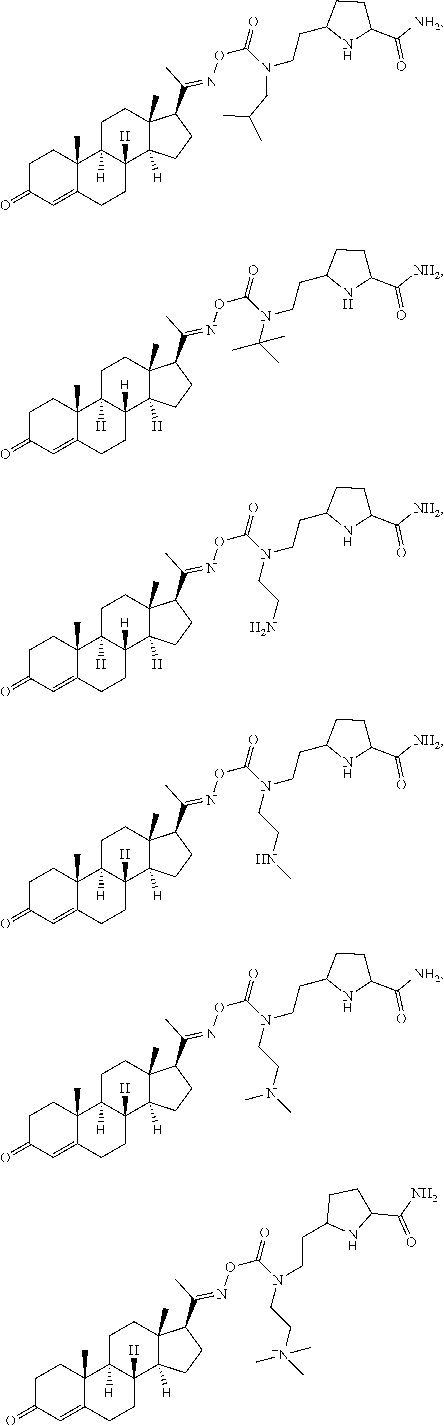

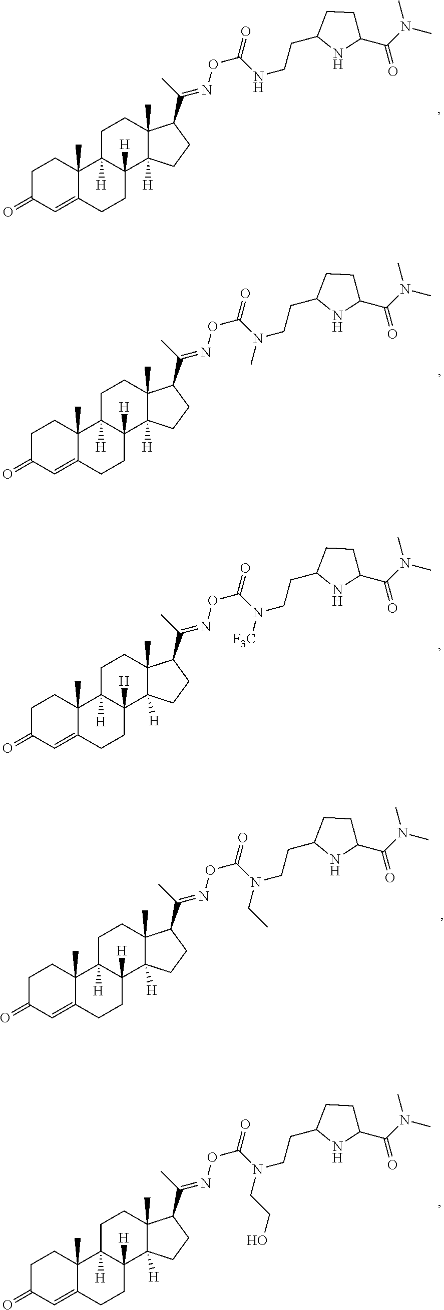

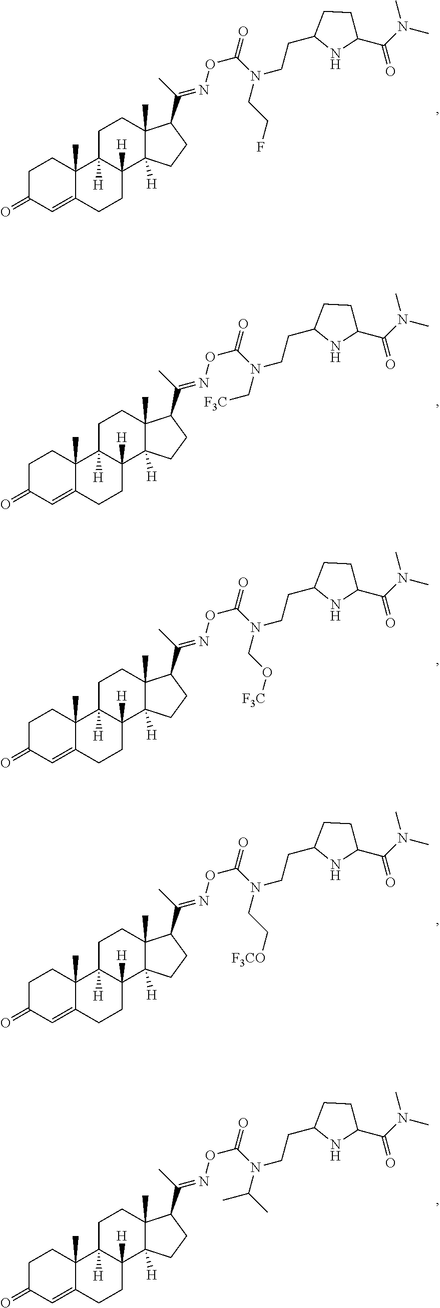

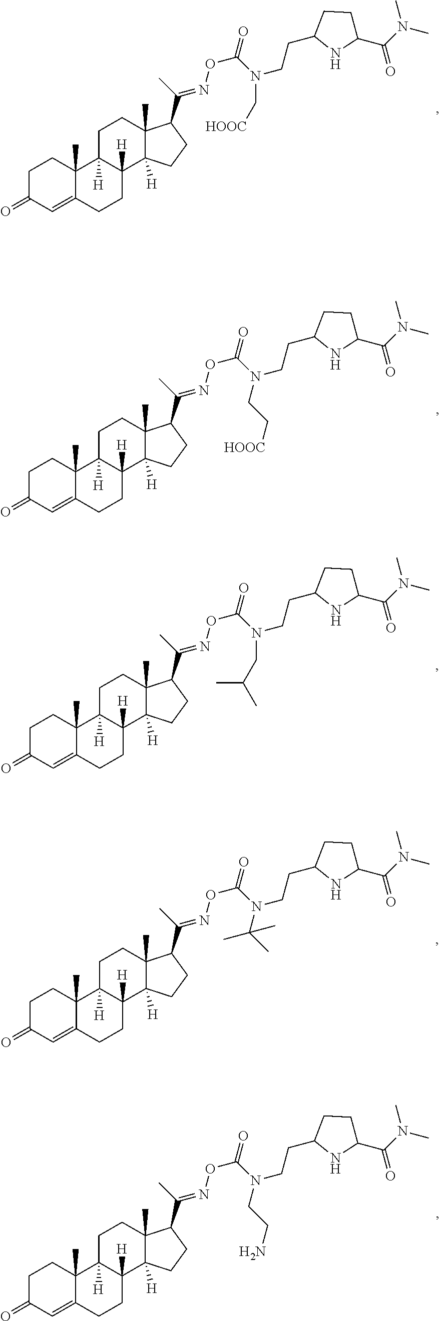

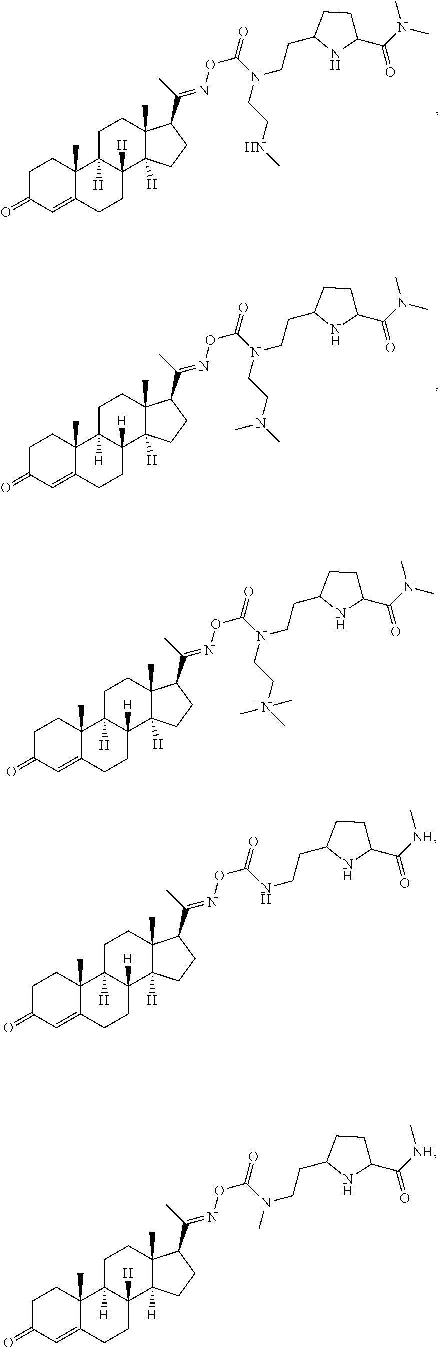

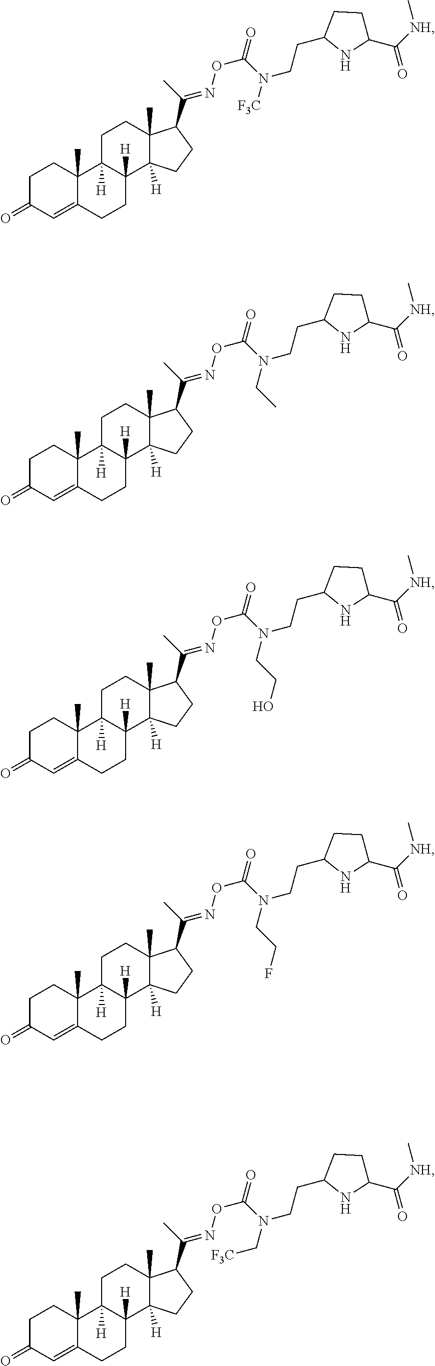

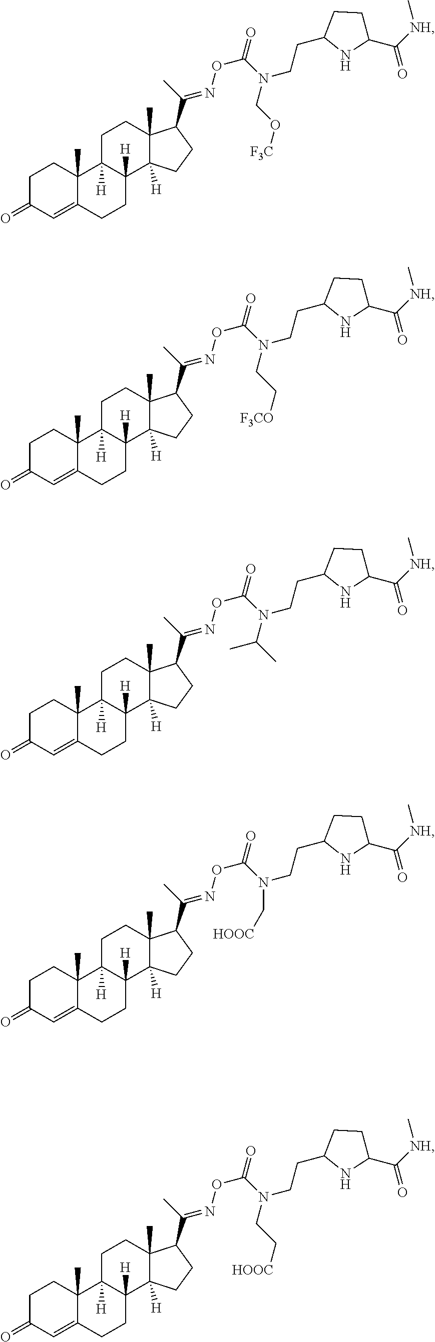

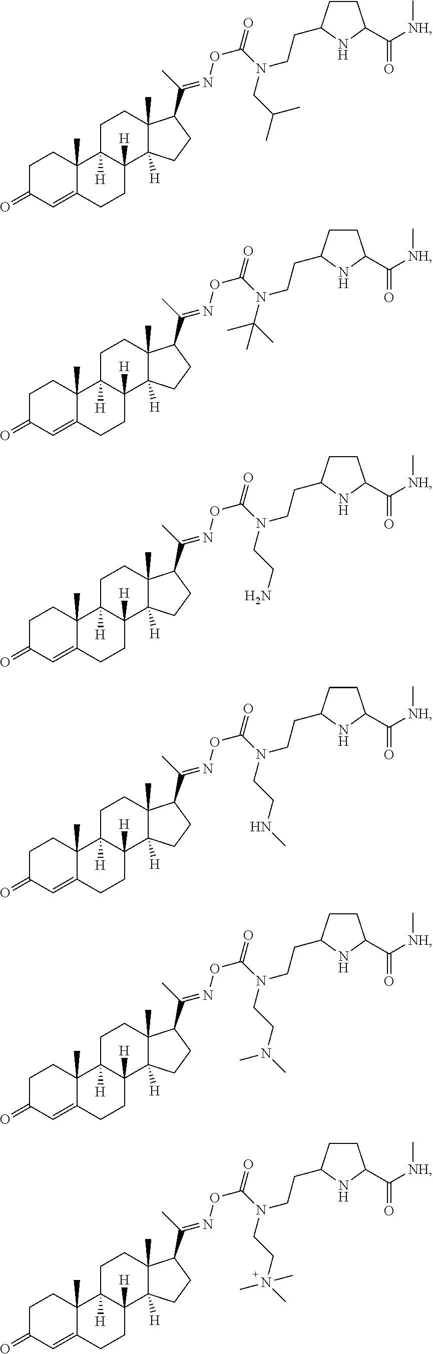

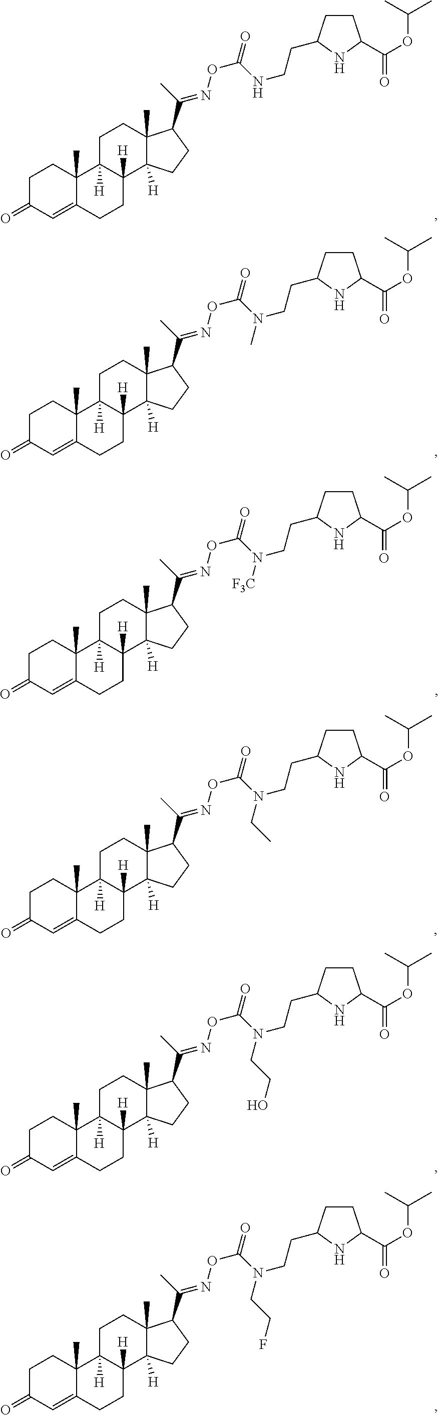

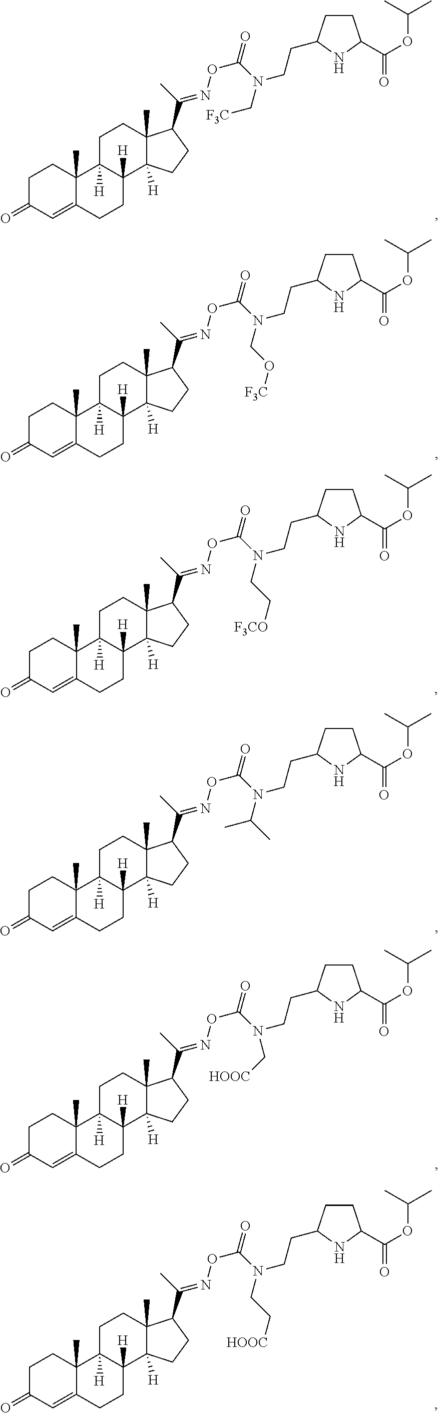

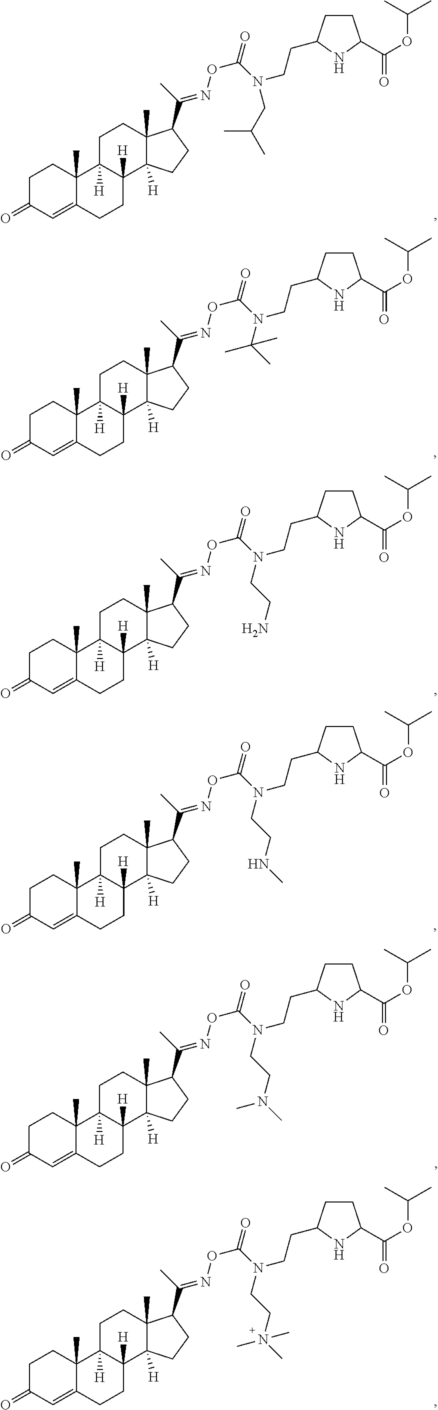

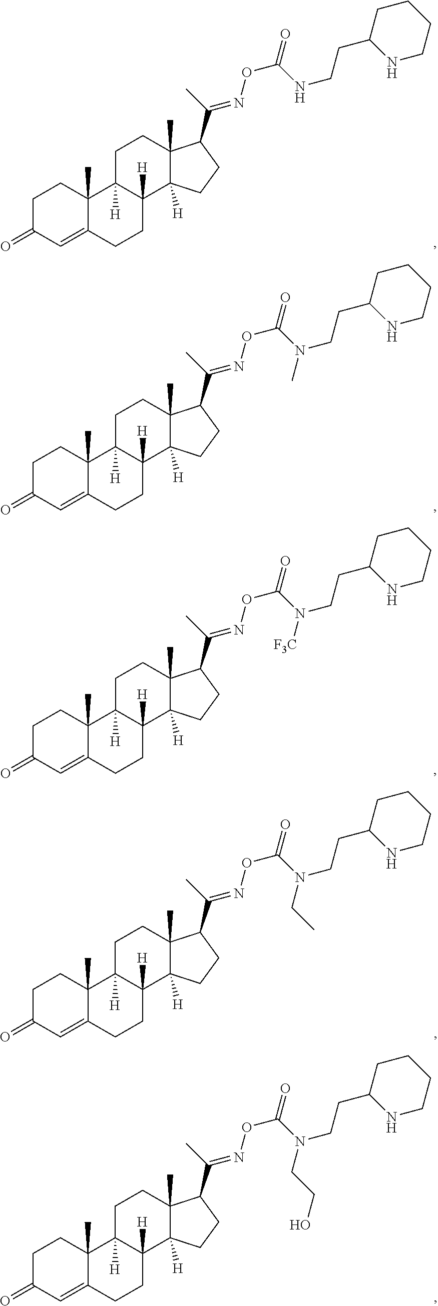

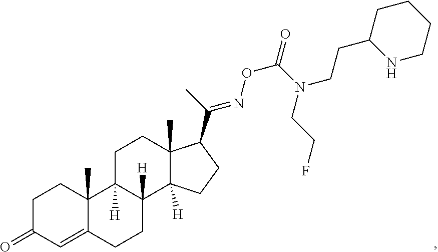

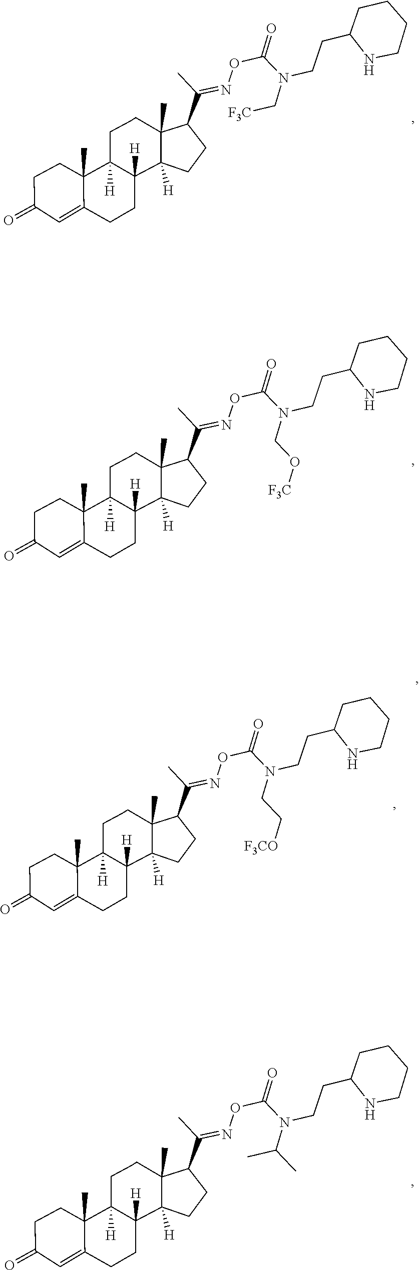

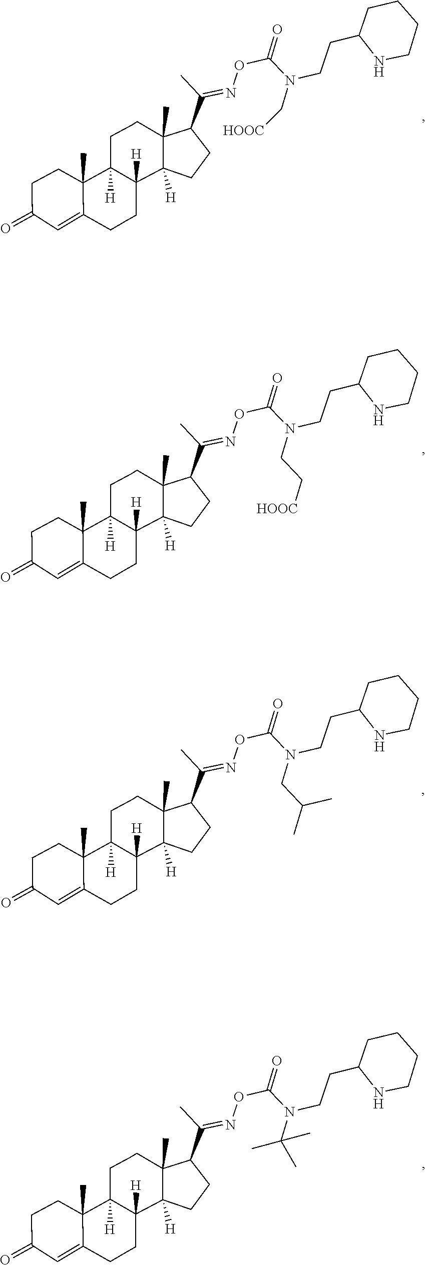

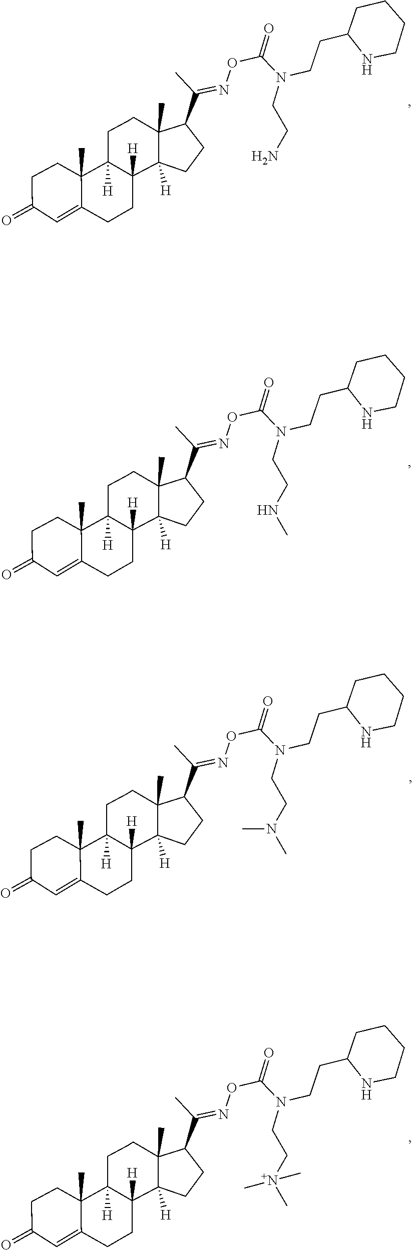

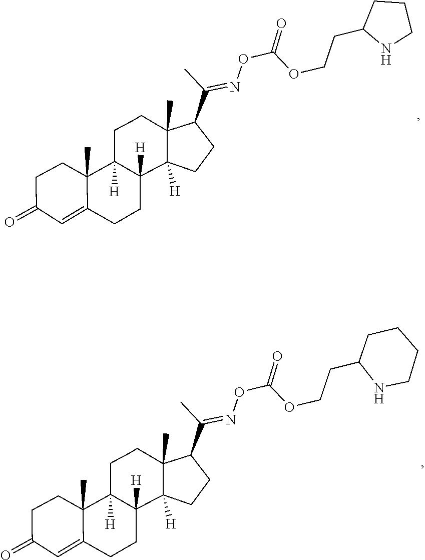

and pharmaceutically acceptable salts thereof. In some embodiments, the ring carbon atom in the pyrrolidine or piperidine moiety that connects to the rest of the structure is in the S configuration. In some embodiments, the ring carbon atom in the pyrrolidine or piperidine moiety that connects to the rest of the structure is in the R configuration.

Exemplary compounds of Formula II-1 also include:

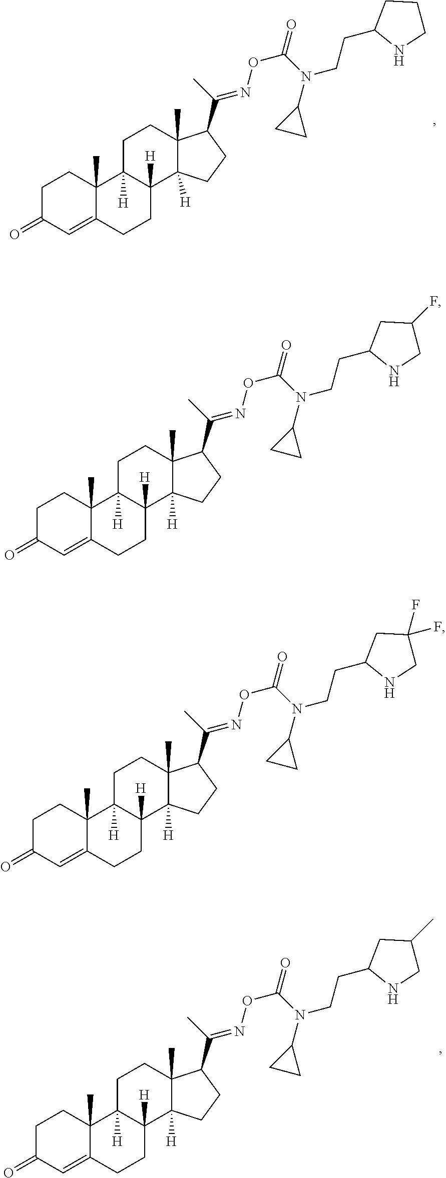

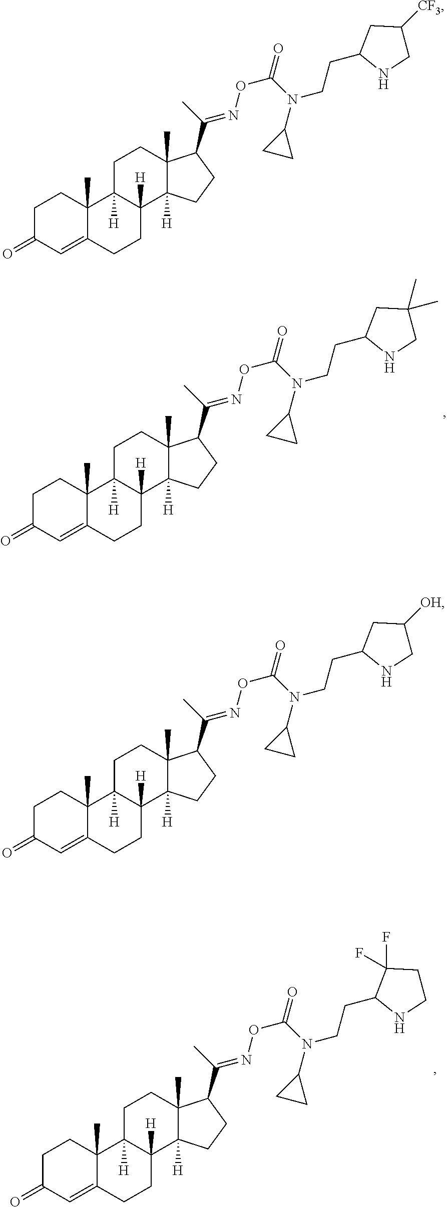

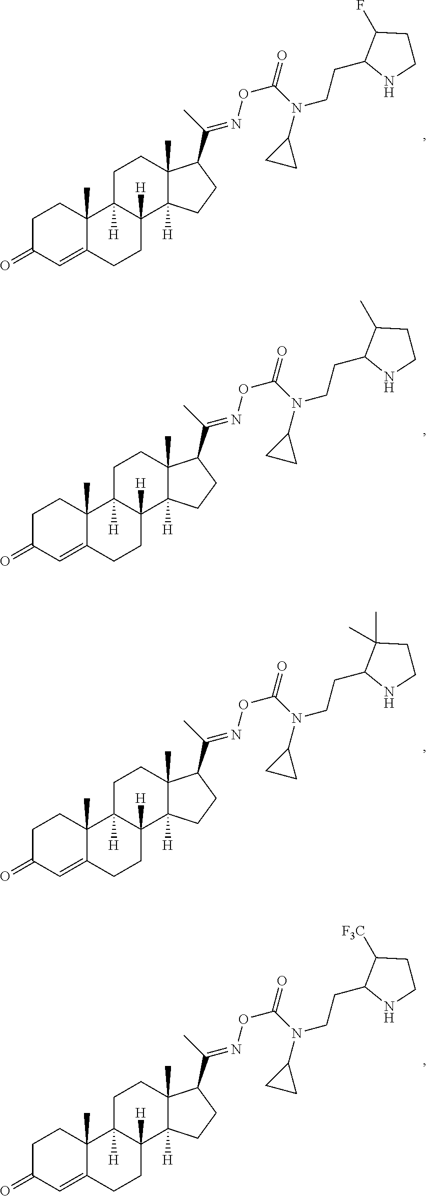

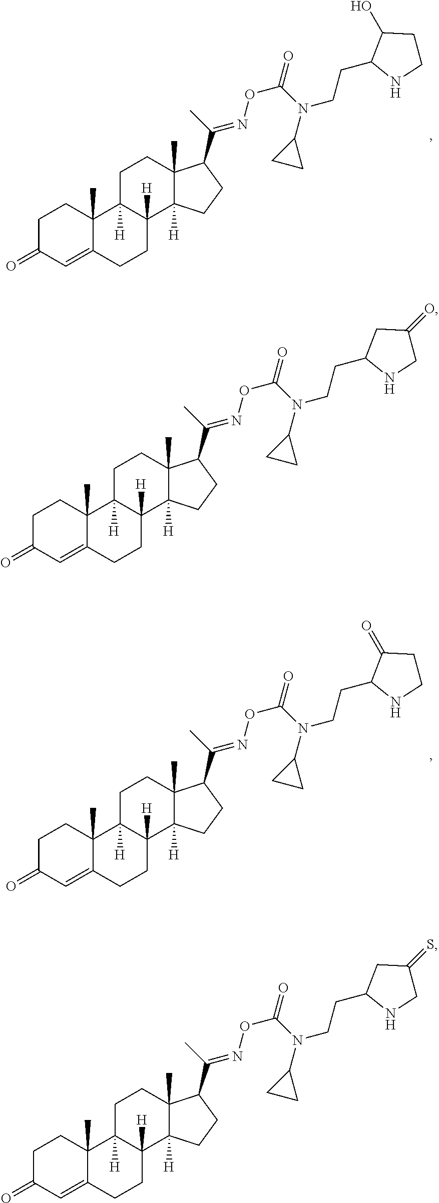

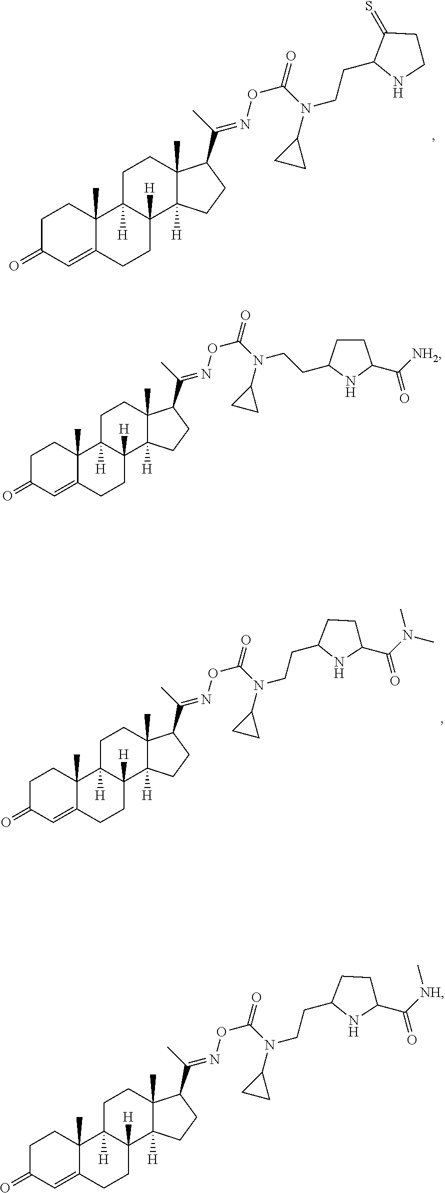

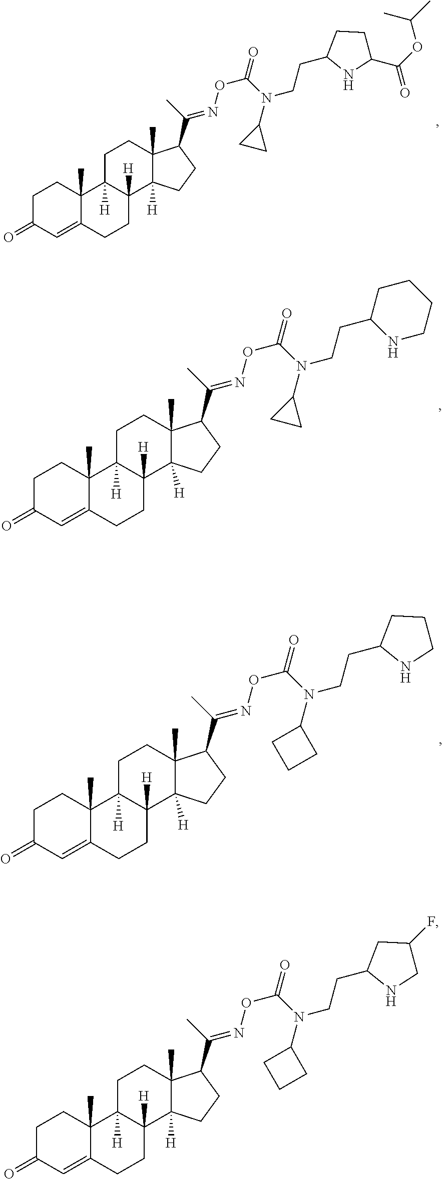

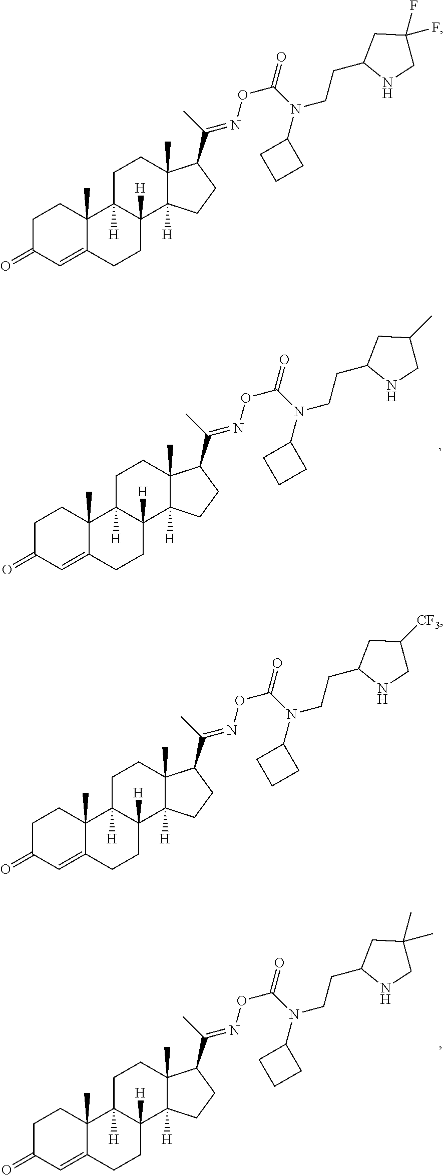

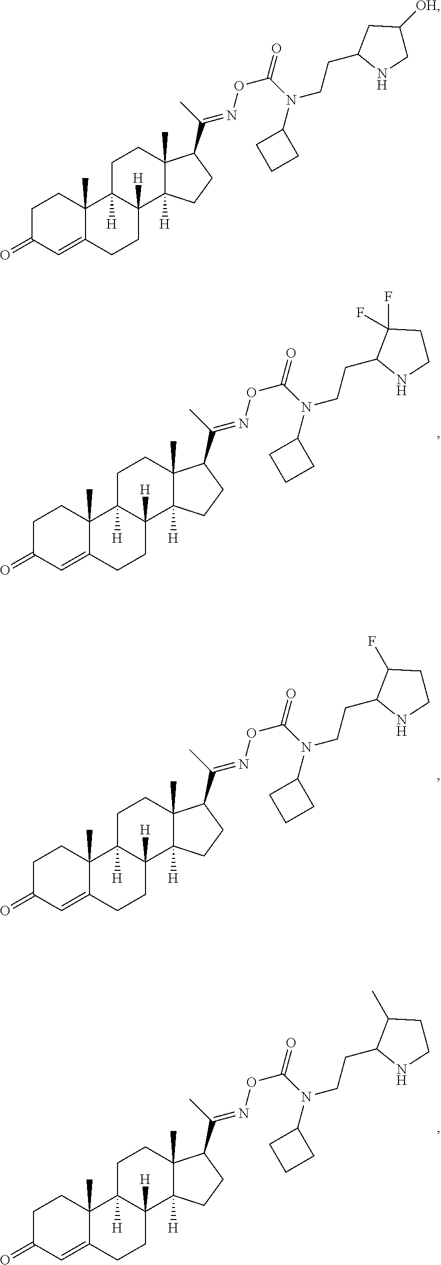

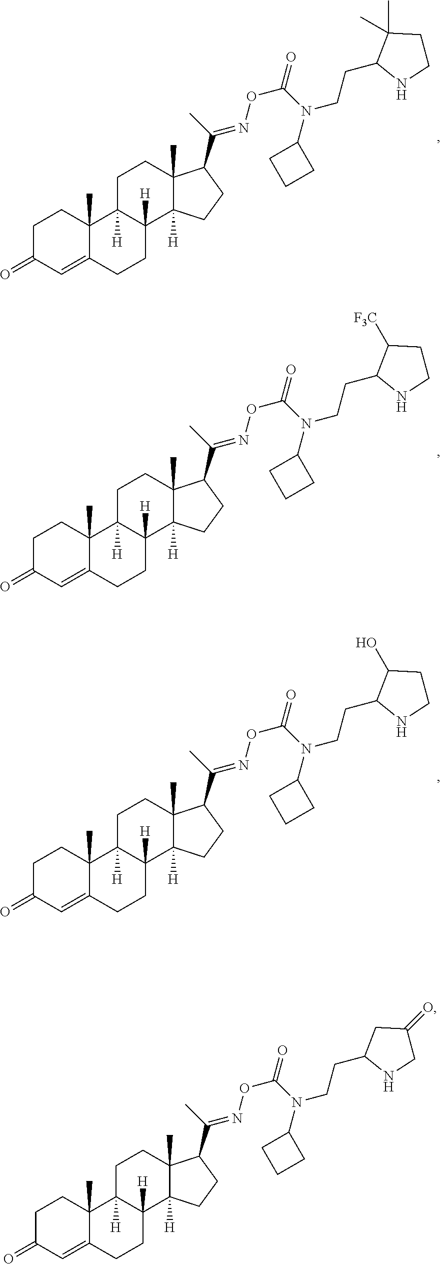

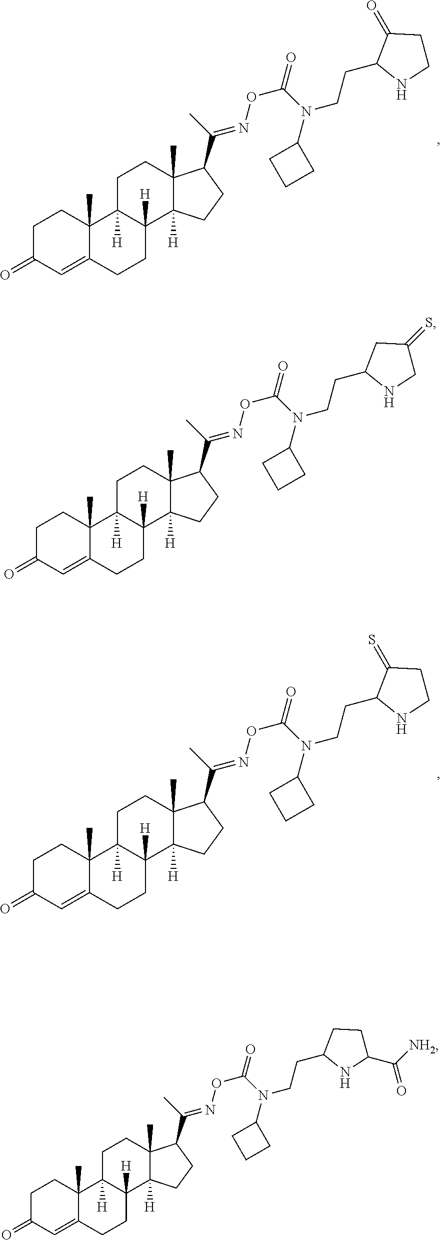

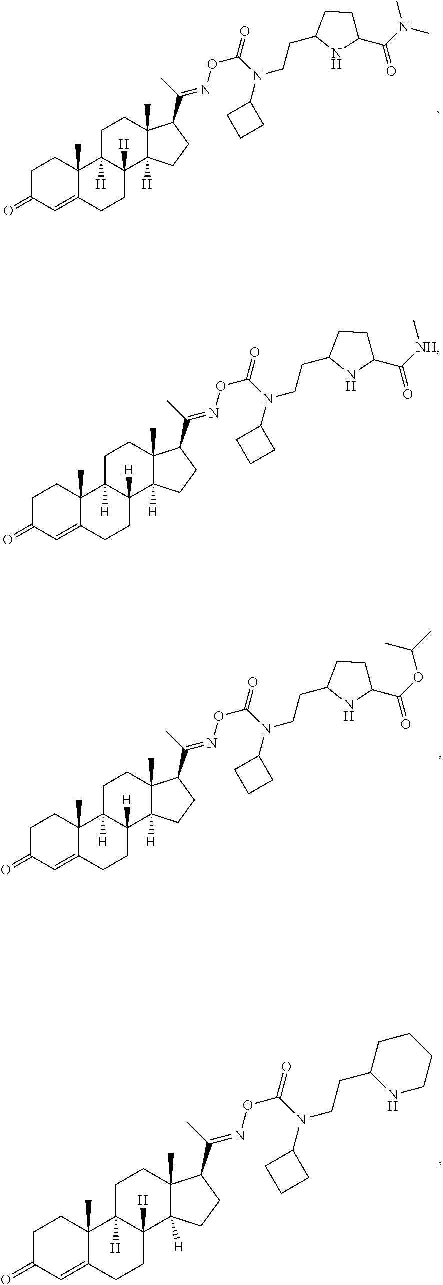

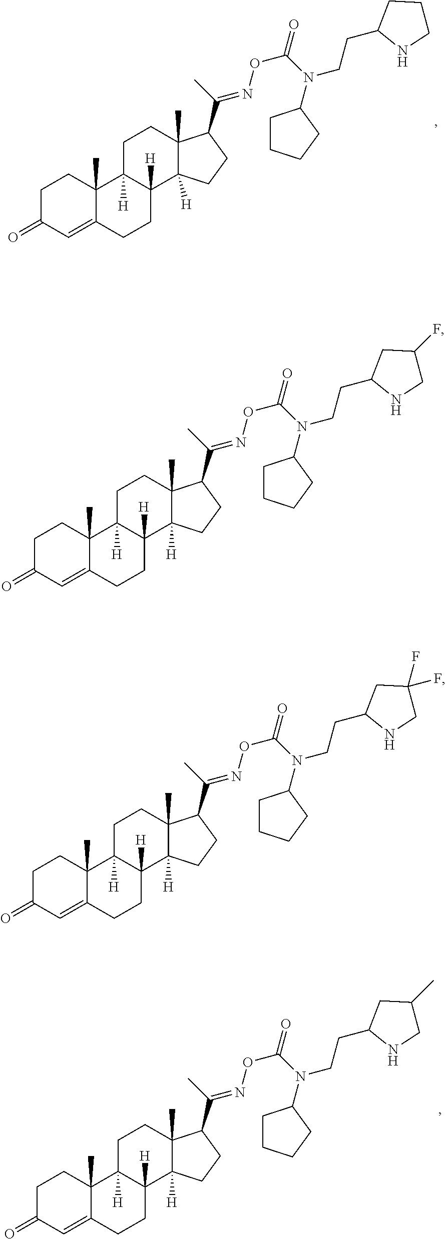

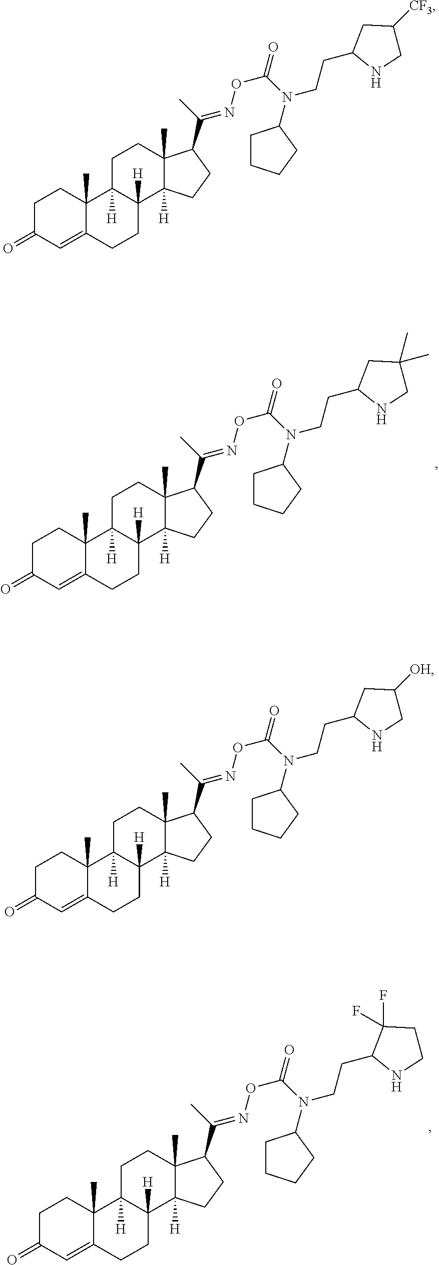

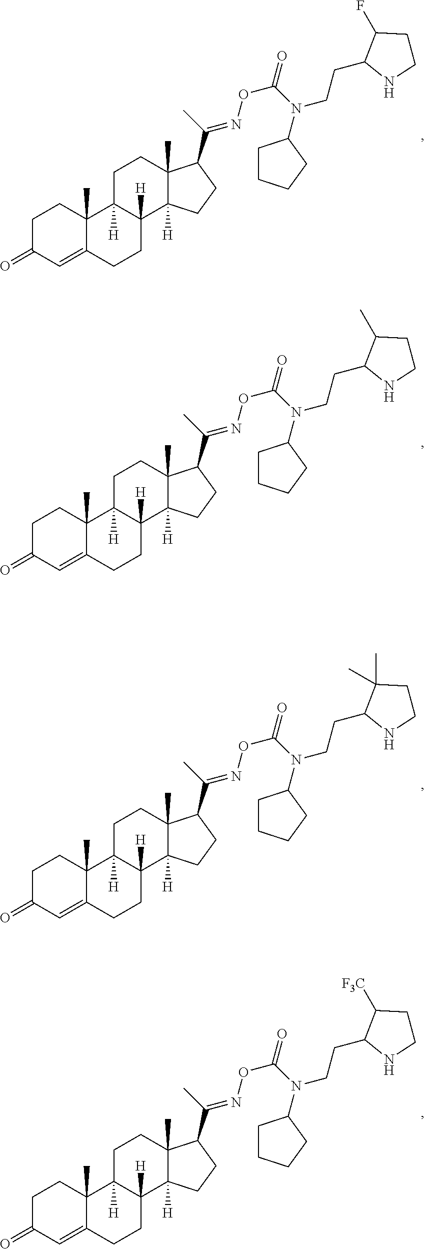

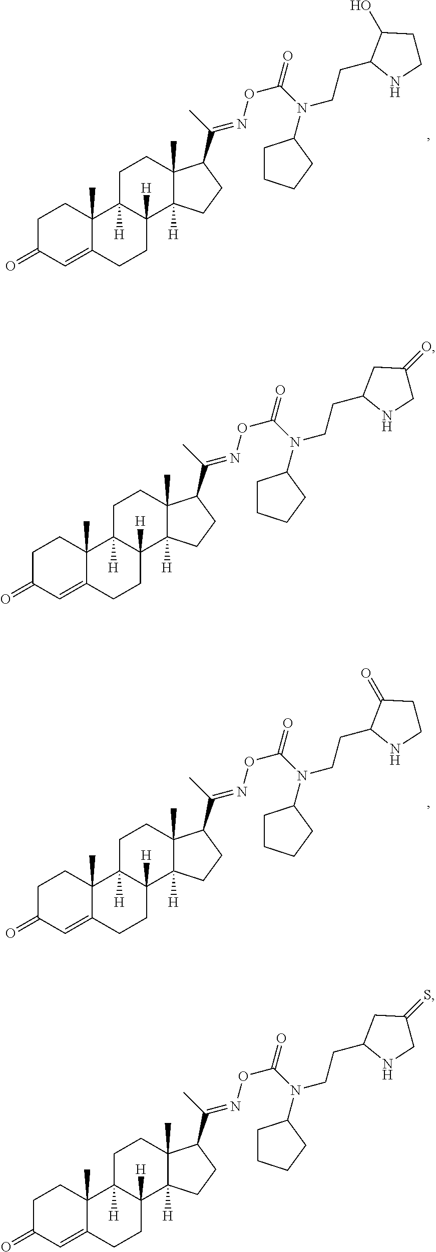

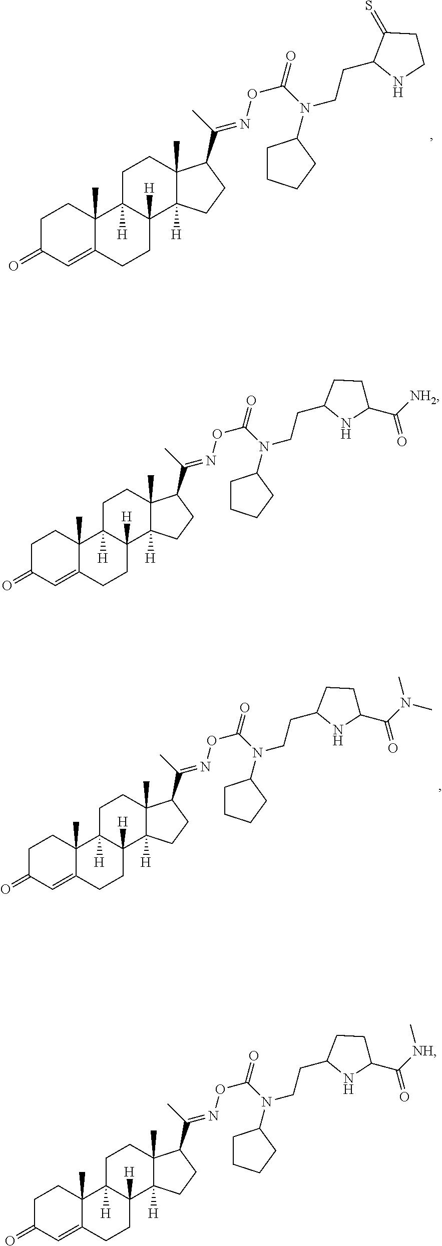

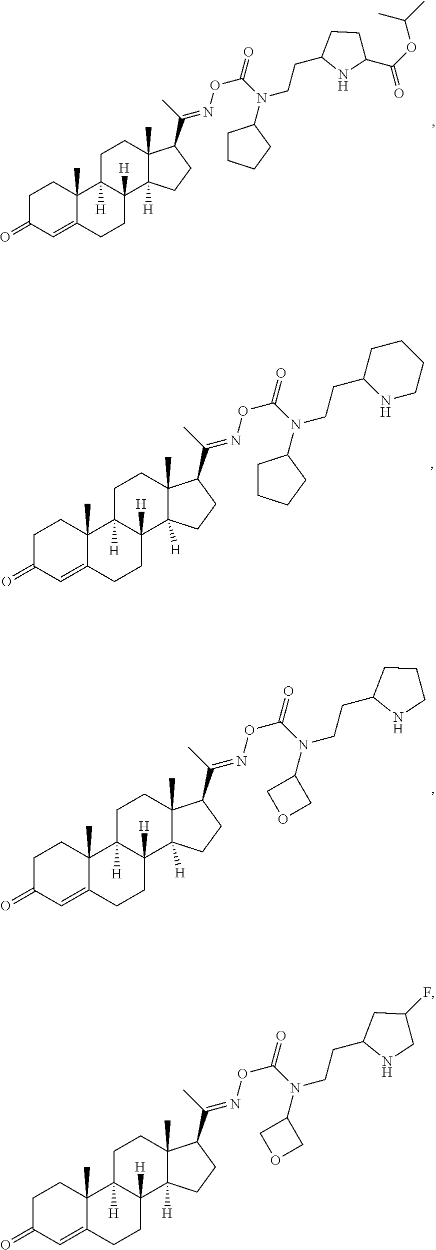

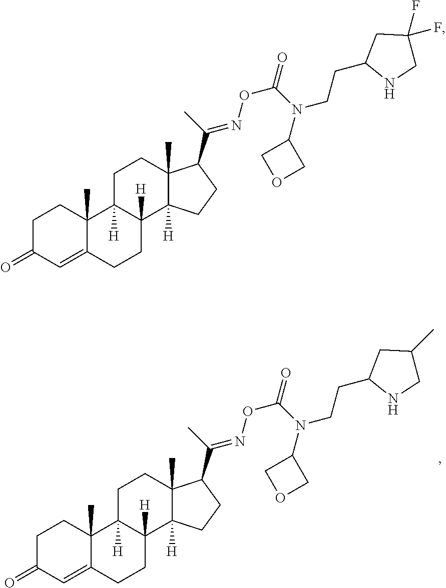

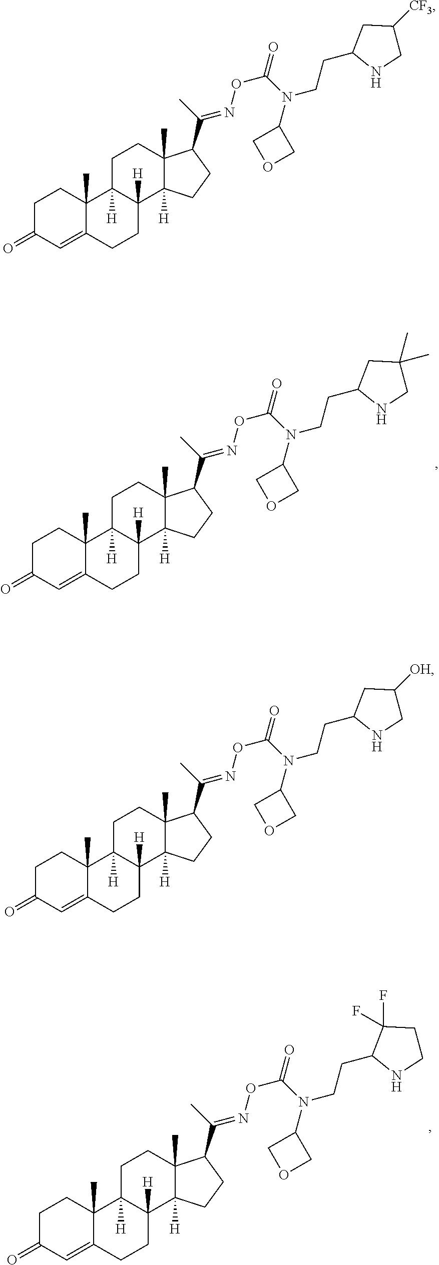

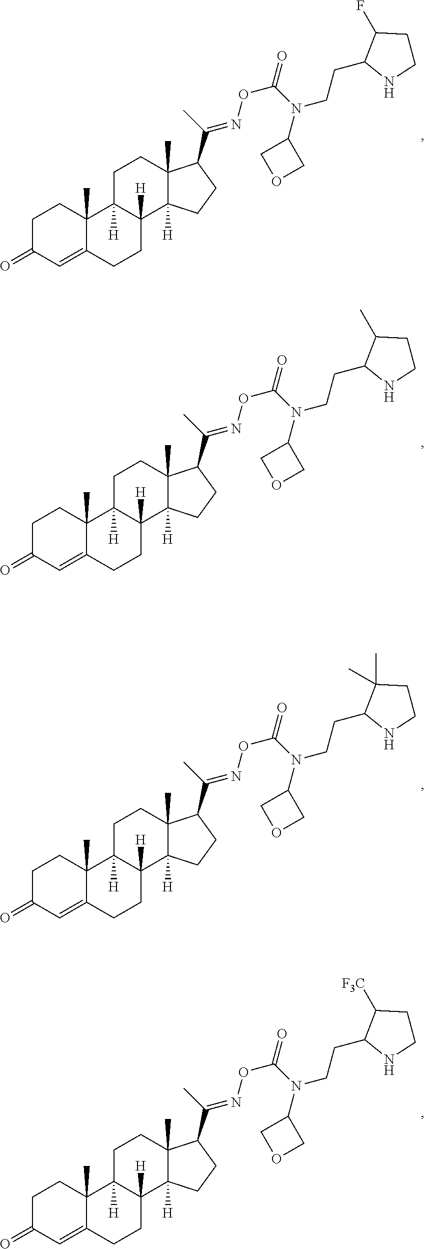

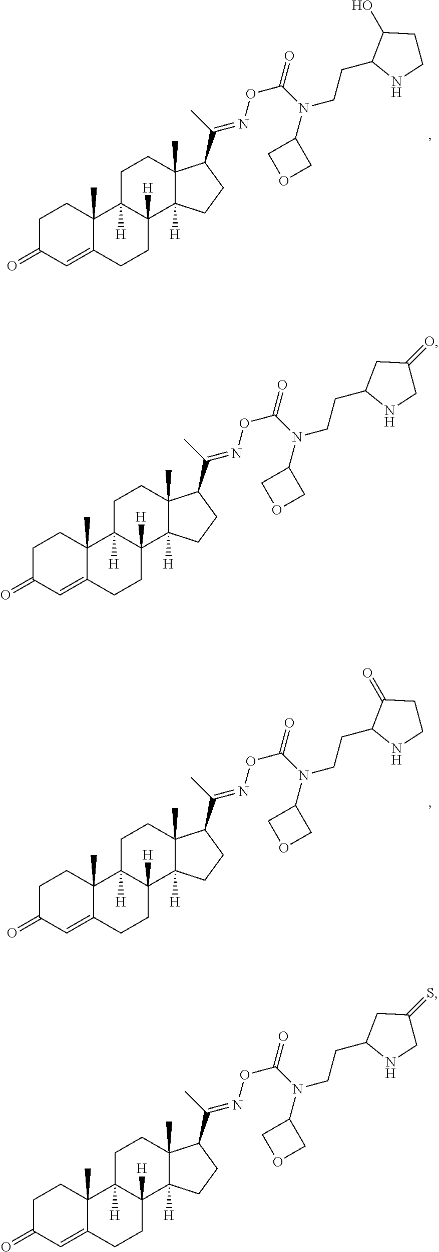

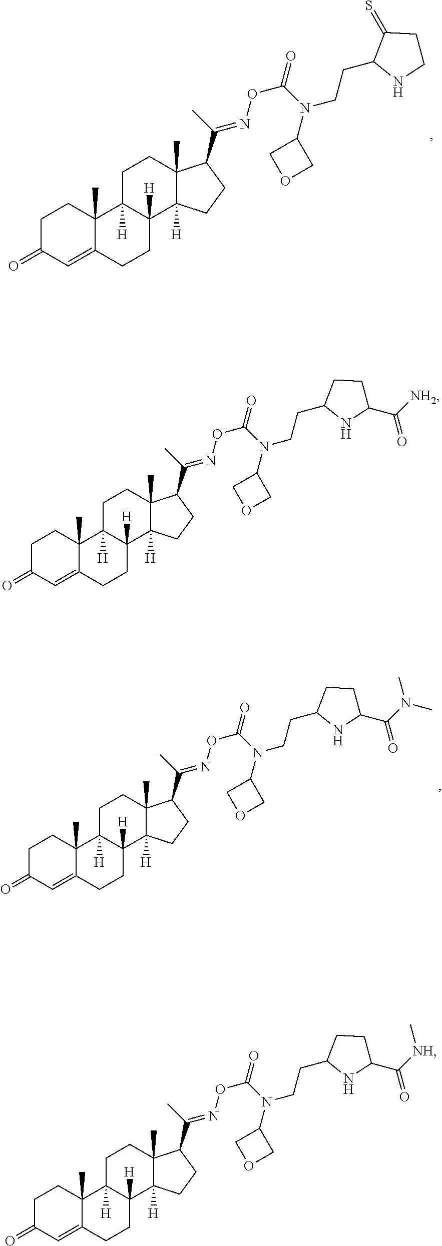

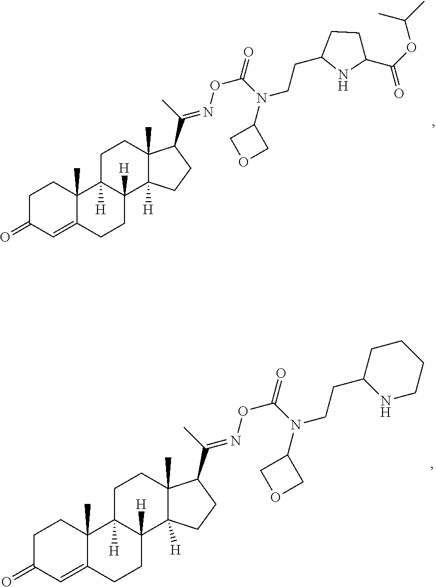

and pharmaceutically acceptable salts thereof. In some embodiments, the ring carbon atom in the pyrrolidine or piperidine moiety that connects to the rest of the structure is in the S configuration. In some embodiments, the ring carbon atom in the pyrrolidine or piperidine moiety that connects to the rest of the structure is in the R configuration.

Exemplary compounds of Formula II-1 also include:

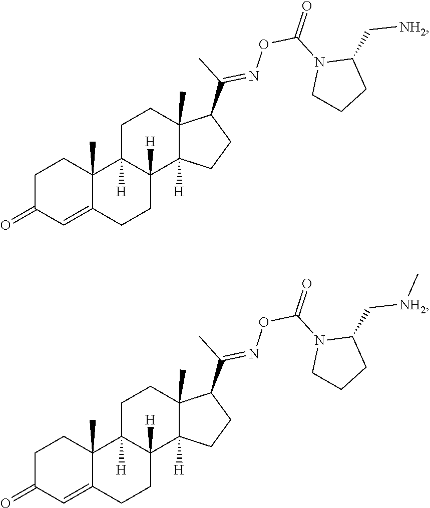

and pharmaceutically acceptable salts thereof.

In some embodiments, the foregoing exemplified compounds are in a non-salt form as shown in the structures. In some embodiments, the compounds are in a salt form. In some embodiments, the compounds are in an HCl salt form.

### C. Properties

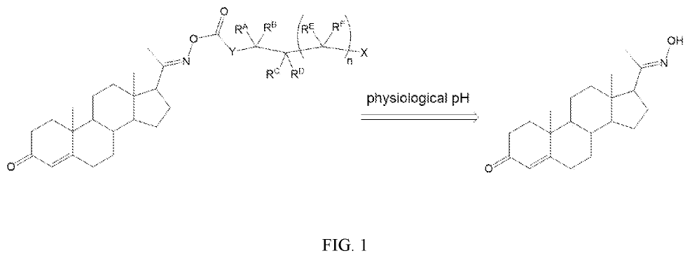

In general, the compounds disclosed herein are highly soluble in an aqueous medium. They may be capable of self-immolative cleavage in response to environmental pH changes, releasing the parent neurosteroid C20-oxime (see FIG. 1 for an exemplary illustration).

In some embodiments, the compounds are stable in an acidic (pH<7) aqueous medium but exhibit a wide range of release kinetics in human plasma.

In some embodiments, the compounds have an aqueous stability, t_{1/2}, at pH 4.0 of at least 90 days, at least 60 days, or at least 30 days. In some embodiments, the compounds have an aqueous stability, t_{1/2}, at pH 4.0 of between 30 days and a year. In some embodiments, the compounds have an aqueous stability, t_{1/2}, at pH 4.0 of between 60 days and a year. In some embodiments, the compounds have an aqueous stability, t_{1/2}, at pH 4.0 of between 90 days and a year. In some embodiments, the compounds have an aqueous stability, t_{1/2}, at pH 4.0 of between 30 days and 180 days. In some embodiments, the compounds have an aqueous stability, t_{1/2}, at pH 4.0 of between 30 days and 150 days. In some embodiments, the compounds have an aqueous stability, t_{1/2}, at pH 4.0 of between 30 days and 120 days. As used herein, the “aqueous plasma stability, t_{1/2}, at pH 4.0” of the compounds is determined in an acetate buffer according to the procedure described in Example 12.

In some embodiments, the compounds have an aqueous stability, t_{1/2}, at pH 5.5 of at least 90 days, at least 60 days, or at least 30 days. In some embodiments, the compounds have an aqueous stability, t_{1/2}, at pH 5.5 of between 30 days and a year. In some embodiments, the compounds have an aqueous stability, t_{1/2}, at pH 5.5 of between 60 days and a year. In some embodiments, the compounds have an aqueous stability, t_{1/2}, at pH 5.5 of between 90 days and a year. In some embodiments, the compounds have an aqueous stability, t_{1/2}, at pH 5.5 of between 30 days and 180 days. In some embodiments, the compounds have an aqueous stability, t_{1/2}, at pH 5.5 of between 30 days and 150 days. In some embodiments, the compounds have an aqueous stability, t_{1/2}, at pH 5.5 of between 30 days and 120 days. As used herein, the “aqueous plasma stability, t_{1/2}, at pH 5.5” of the compounds is determined in an acetate buffer according to the procedure described in Example 12.

In some embodiments, the compounds have a human plasma stability, t_{1/2}, of at most 24 hours, at most 12 hours, at most six hours, at most two hours, or at most one hour. In some embodiments, the compounds have a human plasma stability, t_{1/2}, of between zero and six hours, between zero and five hours, between zero and four hours, between zero and three hours, between zero and two hours, between zero and one hour, or between zero and half an hour. In some embodiments, the compounds have a human plasma stability, t_{1/2}, of between zero and two hours. In some embodiments, the compounds have a human plasma stability, t_{1/2}, of between zero and one hour. In some embodiments, the compounds have a human plasma stability, t_{1/2}, of between zero and half an hour. As used herein, the “human plasma stability, t_{1/2}” of the compounds is determined according to the procedure described in Example 12.

### III. COMPOSITIONS

Disclosed are compositions containing a compound disclosed herein. In some embodiments, the compound in the composition is in greater than 80%, 85%, 90%, or 95% enantiomeric or diastereomeric excess. In some embodiments, the compound in the composition is in greater than 95% enantiomeric or diastereomeric excess.

In some embodiments, the compositions contain a compound having a structure of Formula II or a pharmaceutically acceptable salt, hydrate, or hydrated salt of Formula II, wherein the compound is in greater than 80%, 85%, 90%, or 95% enantiomeric or diastereomeric excess. In some embodiments, the compound is in greater than 95% enantiomeric or diastereomeric excess.

In some embodiments, the compositions contain a compound having a structure of Formula IIA or a pharmaceutically acceptable salt, hydrate, or hydrated salt of Formula IIA, wherein the compound is in greater than 80%, 85%, 90%, or 95% enantiomeric or diastereomeric excess. In some embodiments, the compound is in greater than 95% enantiomeric or diastereomeric excess.

In some embodiments, the compositions contain a compound having a structure of Formula IIB or a pharmaceutically acceptable salt, hydrate, or hydrated salt of Formula IIB, wherein the compound is in greater than 80%, 85%, 90%, or 95% enantiomeric or diastereomeric excess. In some embodiments, the compound is in greater than 95% enantiomeric or diastereomeric excess.

In some embodiments, the compositions contain a compound having a structure of Formula IIC or a pharmaceutically acceptable salt, hydrate, or hydrated salt of Formula IIC, wherein the compound is in greater than 80%, 85%, 90%, or 95% enantiomeric or diastereomeric excess. In some embodiments, the compound is in greater than 95% enantiomeric or diastereomeric excess.

In some embodiments, the compositions contain a compound having a structure of Formula IID or a pharmaceutically acceptable salt, hydrate, or hydrated salt of Formula IID, wherein the compound is in greater than 80%, 85%, 90%, or 95% enantiomeric or diastereomeric excess. In some embodiments, the compound is in greater than 95% enantiomeric or diastereomeric excess.

In some embodiments, the compositions contain a compound having a structure of Formula II-1 or a pharmaceutically acceptable salt, hydrate, or hydrated salt of Formula II-1, wherein the compound is in greater than 80%, 85%, 90%, or 95% enantiomeric or diastereomeric excess for the configuration depicted by Formula II-1. In some embodiments, the compound is in greater than 95% enantiomeric or diastereomeric excess for the configuration depicted by Formula II-1.

In some embodiments, the compositions contain a compound having a structure of Formula II-1A or a pharmaceutically acceptable salt, hydrate, or hydrated salt of Formula II-1A, wherein the compound is in greater than 80%, 85%, 90%, or 95% enantiomeric or diastereomeric excess for the configuration depicted by Formula II-1A. In some embodiments, the compound is in greater than 95% enantiomeric or diastereomeric excess for the configuration depicted by Formula II-1A.

In some embodiments, the compositions contain a compound having a structure of Formula II-1B or a pharmaceutically acceptable salt, hydrate, or hydrated salt of Formula II-1B, wherein the compound is in greater than 80%, 85%, 90%, or 95% enantiomeric or diastereomeric excess for the configuration depicted by Formula II-1B. In some embodiments, the compound is in greater than 95% enantiomeric or diastereomeric excess for the configuration depicted by Formula II-1B.

In some embodiments, the compositions contain a compound having a structure of Formula II-1C or a pharmaceutically acceptable salt, hydrate, or hydrated salt of Formula II-1C, wherein the compound is in greater than 80%, 85%, 90%, or 95% enantiomeric or diastereomeric excess for the configuration depicted by Formula II-1C. In some embodiments, the compound is in greater than 95% enantiomeric or diastereomeric excess for the configuration depicted by Formula II-1C.

In some embodiments, the compositions contain a compound having a structure of Formula II-1D or a pharmaceutically acceptable salt, hydrate, or hydrated salt of Formula II-1D, wherein the compound is in greater than 80%, 85%, 90%, or 95% enantiomeric or diastereomeric excess for the configuration depicted by Formula II-1D. In some embodiments, the compound is in greater than 95% enantiomeric or diastereomeric excess for the configuration depicted by Formula II-1D.

The disclosed compounds may be present in a mixture of a salt form and a non-salt form. In some embodiments, more than 50%, 60%, 70%, 80%, 90%, 95%, or 98% of the compound in the mixture may be in the non-salt form, calculated as the ratio of the weight of the non-salt form to the total weight of the mixture. In some embodiments, more than 90% of the compound in the mixture may be in the non-salt form. In some embodiments, more than 50%, 60%, 70%, 80%, 90%, 95%, or 98% of the compound in the mixture may be in the salt form, calculated as the ratio of the weight of the salt form to the total weight of the mixture. In some embodiments, more than 90% of the compound in the mixture may be in the salt form. In some embodiments, more than 50%, 60%, 70%, 80%, 90%, 95%, or 98% of the compound in the mixture may be in an HCl salt form, calculated as the ratio of the weight of the HCl salt form to the total weight of the mixture. In some embodiments, more than 90% of the compound in the mixture may be in the HCl salt form.

### IV. FORMULATIONS

Disclosed are pharmaceutical formulations containing a compound or composition described herein. Generally, the pharmaceutical formulations also contain one or more pharmaceutically acceptable excipients.

The pharmaceutical formulations can be in a form chosen from tablets, capsules, caplets, pills, powders, beads, granules, particles, creams, gels, solutions (such as aqueous solutions, e.g., buffer, saline, and buffered saline), emulsions, suspensions (including nano- and micro-suspensions), nanoparticulate formulations, etc. In some embodiments, the pharmaceutical formulations are formulated for oral administration. In some embodiments, the pharmaceutical formulations are formulated for intravenous administration. In some embodiments, the pharmaceutical formulations are formulated for intramuscular administration.

As used herein, “emulsion” refers to a mixture of non-miscible components homogenously blended together. In some forms, the non-miscible components include a lipophilic component and an aqueous component. For example, an emulsion may be a preparation of one liquid distributed in small globules throughout the body of a second liquid. The dispersed liquid is the discontinuous phase, and the dispersion medium is the continuous phase. When oil or an oleaginous substance is the dispersed liquid and water or an aqueous solution is the continuous phase, it is known as an oil-in-water emulsion, whereas when water or an aqueous solution is the dispersed phase and oil or an oleaginous substance is the continuous phase, it is known as a water-in-oil emulsion.

As used herein, “biocompatible” refers to materials that are neither themselves toxic to the host (e.g., a non-human animal or human), nor degrade (if the material degrades) at a rate that produces monomeric or oligomeric subunits or other byproducts at toxic concentrations in the host.

As used herein, “biodegradable” refers to degradation or breakdown of a polymeric material into smaller (e.g., non-polymeric) subunits or digestion of the material into smaller subunits.

As used herein, “enteric polymers” refers to polymers that become soluble in the higher pH environment of the lower gastrointestinal tract or slowly erode as they pass through the gastrointestinal tract.

As used herein, “nanoparticulate formulations” generally refers to formulations containing nanoparticles, which are particles having a diameter from about 1 nm to about 1000 nm, from about 10 nm to about 1000 nm, from about 100 nm to about 1000 nm, or from about 250 nm to about 1000 nm. In some embodiments, “nanoparticulate formulations” can also refer to formulations containing microparticles, which are particles having a diameter from about 1 micron to about 100 microns, from about 1 to about 50 microns, from about 1 to about 30 microns, from about 1 micron to about 10 microns. In some embodiments, the nanoparticulate formulation may contain a mixture of nanoparticles, as defined above, and microparticles, as defined above.

As used herein, “surfactant” refers to any agent which preferentially absorbs to an interface between two immiscible phases, such as the interface between water (or aqueous solution) and an organic solvent (or organic solution), between water (or aqueous solution) and air, or between organic solvent (or organic solution) and air. Surfactants generally possess a hydrophilic moiety and a lipophilic moiety.

As used herein, “gel” is a semisolid system containing a dispersion of the active ingredient, i.e., a compound or composition according to the present disclosure, in a liquid vehicle that is rendered semisolid by the action of a thickening agent or polymeric material dissolved or suspended in the liquid vehicle. The liquid vehicle may include a lipophilic component, an aqueous component or both.

As used herein, “hydrogel” refers to a swollen, water-containing network of finely dispersed polymer chains that are water-insoluble, where the polymer molecules are in the external or dispersion phase and water (or an aqueous solution) forms the internal or dispersed phase. The polymer chains can be chemically cross-linked (chemical gels) or physically cross-linked (physical gels). Chemical gels possess polymer chains connected through covalent bonds, whereas physical gels have polymer chains linked by non-covalent interactions, such as van der Waals interactions, ionic interactions, hydrogen bonding interactions, and hydrophobic interactions.

As used herein, “beads” refers to beads made with the active ingredient (i.e., a compound or composition according to the present disclosure) and one or more pharmaceutically acceptable excipients. The beads can be produced by applying the active ingredient to an inert support, e.g., inert sugar core coated with the active ingredient. Alternatively, the beads can be produced by creating a “core” comprising both the active ingredient and at least one of the one or more pharmaceutically acceptable excipients. As used herein, “granules” refers to a product made by processing particles of the active ingredient (i.e., a compound or composition according to the present disclosure) that may or may not include one or more pharmaceutical acceptable excipients. Typically, granules do not contain an inert support and are bigger in size compared to the particles used to produce them. Although beads, granules and particles may be formulated to provide immediate release, beads and granules are usually employed to provide delayed release.

As used herein, “enzymatically degradable polymers” refers to polymers that are degraded by bacterial enzymes present in the intestines and/or lower gastrointestinal tract.

### A. Physical Forms and Unit Dosages

Depending upon the administration route, the compounds or compositions described herein may be formulated in a variety of ways. The pharmaceutical formulations can be prepared in various forms, such as tablets, capsules, caplets, pills, granules, powders, nanoparticle formulations, solutions (such as aqueous solutions, e.g., buffer, saline, and buffered saline), suspensions (including nano- and micro-suspensions), emulsions, creams, gels, and the like.

In some embodiments, the pharmaceutical formulations are in a solid dosage form suitable for simple administration of precise dosages. For example, the solid dosage form may be selected from tablets, soft or hard gelatin or non-gelatin capsules, and caplets for oral administration. Optionally, the solid dosage form is a lyophilized powder that can be readily dissolved and converted to a liquid dosage form for intravenous or intramuscular administration. In some embodiments, the lyophilized powder is manufactured by dissolving the active ingredient (i.e., a compound or composition disclosed herein) in an aqueous medium followed by lyophilization. In some embodiments, the aqueous medium is water, normal saline, PBS, or an acidic aqueous medium such as an acetate buffer.

In some embodiments, the pharmaceutical formulations are in a liquid dosage form suitable for intravenous or intramuscular administration. Exemplary liquid dosage forms include, but are not limited to, solutions, suspensions, and emulsions. In some embodiments, the pharmaceutical formulations are in the form of a sterile aqueous solution. In some embodiments, the sterile aqueous solution is a sterile normal saline solution. In some embodiments, the sterile aqueous solution is a sterile PBS solution. In some embodiments, the sterile aqueous solution is an acidic, sterile aqueous solution such as a sterile acetate buffer. In some embodiments, the sterile aqueous solution is manufactured by dissolving a lyophilized powder containing the active ingredient (i.e., a compound or composition disclosed herein) in an aqueous medium. For example, the sterile aqueous solution can be prepared by dissolving the lyophilized powder containing the active ingredient in a dose-appropriate volume of sterile water, sterile normal saline, sterile PBS, or acidic, sterile aqueous medium such as a sterile acetate buffer. In some embodiments, the lyophilized powder containing the active ingredient is the same as those described in the paragraph above.

In some embodiments, the pharmaceutical formulations are in a unit dosage form, and may be suitably packaged, for example, in a box, blister, vial, bottle, syringe, sachet, ampoule, or in any other suitable single-dose or multi-dose holder or container, optionally with one or more leaflets containing product information and/or instructions for use.

### B. Pharmaceutically Acceptable Excipients

Exemplary pharmaceutically acceptable excipients include, but are not limited to, diluents, binders, lubricants, disintegrants, pH-modifying or buffering agents, salts (such as NaCl), preservatives, antioxidants, solubility enhancers, wetting or emulsifying agents, plasticizers, colorants (such as pigments and dyes), flavoring or sweetening agents, thickening agents, emollients, humectants, stabilizers, glidants, solvents or dispersion mediums, surfactants, pore formers, and coating or matrix materials.

In some embodiments, the powders described herein, including the lyophilized powders, contain one or more of the following pharmaceutically acceptable excipients: pH-modifying or buffering agents, salts (such as NaCl), and preservatives.

In some embodiments, the tablets, beads, granules, and particles described herein contain one or more of the following pharmaceutically acceptable excipients: coating or matrix materials, diluents, binders, lubricants, disintegrants, pigments, stabilizers, and surfactants. If desired, the tablets, beads, granules, and particles may also contain a minor amount of nontoxic auxiliary substances such as wetting or emulsifying agents, dyes, pH-buffering agents, and preservatives.

Examples of the coating or matrix materials include, but are not limited to, cellulose polymers (such as methylcellulose, ethyl cellulose, cellulose acetate, cellulose acetate phthalate, hydroxypropyl cellulose, hydroxyethyl cellulose, hydroxymethyl cellulose, hydroxypropyl methylcellulose, hydroxypropyl methylcellulose phthalate, hydroxypropyl methylcellulose acetate succinate, cellulose acetate trimellitate, and carboxymethylcellulose sodium), vinyl polymers and copolymers (such as polyvinyl pyrrolidone, polyvinyl acetate, polyvinyl acetate phthalate, vinyl acetate-crotonic acid copolymer, and ethylene-vinyl acetate copolymer), acrylic acid polymers and copolymers (such as those formed from acrylic acid, methacrylic acid, methyl acrylate, ethyl acrylate, methyl methacrylate, or ethyl methacrylate, as well as methacrylic resins that are commercially available under the tradename EUDRAGIT®), enzymatically degradable polymers (such as azo polymers, pectin, chitosan, amylose, and guar gum), zein, shellac, and polysaccharides. In some embodiments, the coating or matrix materials may contain one or more excipients such as plasticizers, colorants, glidants, stabilizers, pore formers, and surfactants.

In some embodiments, the coating or matrix materials are pH-sensitive or pH-responsive polymers, such as the enteric polymers commercially available under the tradename EUDRAGIT®. For example, EUDRAGIT® L30D-55 and L100-55 are soluble at pH 5.5 and above; EUDRAGIT® L100 is soluble at pH 6.0 and above; EUDRAGIT® S is soluble at pH 7.0 and above.

In some embodiments, the coating or matrix materials are water-insoluble polymers having different degrees of permeability and expandability, such as EUDRAGIT® NE, RL, and RS.

Depending on the coating or matrix materials, the decomposition/degradation or structural change of the pharmaceutical formulations may occur at different locations of the gastrointestinal tract. In some embodiments, the coating or matrix materials are selected such that the pharmaceutical formulations can survive exposure to gastric acid and release the active ingredient in the intestines after oral administration.

Diluents can increase the bulk of a solid dosage formulation so that a practical size is provided for compression of tablets or formation of beads, granules, or particles. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate, powdered sugar, and combinations thereof.

Binders are used to impart cohesive qualities to a solid dosage formulation, and thus ensure that a tablet, bead, granule, or particle remains intact after the formation of the solid dosage formulation. Suitable binders include, but are not limited to, starch, pregelatinized starch, gelatin, sugars (such as sucrose, glucose, dextrose, lactose, and sorbitol), polyethylene glycol, waxes, natural and synthetic gums (such as acacia, tragacanth, and sodium alginate), cellulose (such as hydroxypropylmethylcellulose, hydroxypropylcellulose, and ethylcellulose), veegum, and synthetic polymers (such as acrylic acid copolymers, methacrylic acid copolymers, methyl methacrylate copolymers, aminoalkyl methacrylate copolymers, polyacrylic acid, polymethacrylic acid, and polyvinylpyrrolidone), and combinations thereof.

Lubricants are used to facilitate tablet manufacture. Suitable lubricants include, but are not limited to, magnesium stearate, calcium stearate, stearic acid, glycerol behenate, polyethylene glycol, talc, and mineral oil.

Disintegrants are used to facilitate disintegration or “breakup” of a solid dosage formulation after administration. Suitable disintegrants include, but are not limited to, starch, sodium starch glycolate, sodium carboxymethyl starch, sodium carboxymethylcellulose, hydroxypropyl cellulose, pregelatinized starch, clays, cellulose, gums, and cross-linked polymers, such as cross-linked polyvinylpyrrolidone (e.g., POLYPLASDONE® XL).

Plasticizers are normally present to produce or promote plasticity and flexibility and to reduce brittleness. Examples of plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil, and acetylated monoglycerides.

Stabilizers are used to inhibit or retard decomposition reactions of the active ingredient in the pharmaceutical formulations or stabilize particles in a dispersion. For example, when the decomposition reactions involve an oxidation reaction of the active ingredient in the pharmaceutical formulations, the stabilizer can be an antioxidant or a reducing agent. Stabilizers also include nonionic emulsifiers such as sorbitan esters, polysorbates, and polyvinylpyrrolidone.

Glidants are used to reduce sticking effects during film formation and drying. Exemplary glidants include, but are not limited to, talc, magnesium stearate, and glycerol monostearates.

Preservatives can inhibit the deterioration and/or decomposition of a pharmaceutical formulation. Deterioration or decomposition can be brought about by one or more of microbial growth, fungal growth, and undesirable chemical or physical changes. Suitable preservatives include benzoate salts (e.g., sodium benzoate), ascorbic acid, methyl hydroxybenzoate, ethyl p-hydroxybenzoate, n-propyl p-hydroxybenzoate, n-butyl p-hydroxybenzoate, potassium sorbate, sorbic acid, propionate salts (e.g., sodium propionate), chlorobutanol, benzyl alcohol, and combinations thereof.

Surfactants may be anionic, cationic, amphoteric, or nonionic surface-active agents. Exemplary anionic surfactants include, but are not limited to, those containing a carboxylate, sulfonate, or sulfate ion. Examples of anionic surfactants include sodium, potassium, and ammonium salts of long-chain (e.g., 13-21) alkyl sulfonates (such as sodium lauryl sulfate), alkylaryl sulfonates (such as sodium dodecylbenzene sulfonate), and dialkyl sulfosuccinates (such as sodium bis-(2-ethylthioxyl)-sulfosuccinate). Examples of cationic surfactants include, but are not limited to, quaternary ammonium compounds such as benzalkonium chloride, benzethonium chloride, cetrimonium bromide, stearyl dimethylbenzyl ammonium chloride, polyoxyethylene, and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, poloxamers (such as poloxamer 401), stearoyl monoisopropanolamide, and polyoxyethylene hydrogenated tallow amide. Examples of amphoteric surfactants include, but are not limited to, sodium N-dodecyl-β-alanine, sodium N-lauryl-β-iminodipropionate, myristoamphoacetate, lauryl betaine, and lauryl sulfobetaine.

Pharmaceutical formulations in the liquid dosage forms typically contain a solvent or dispersion medium such as water, aqueous solution (e.g., buffer, saline, buffered saline), ethanol, polyol (such as glycerol, propylene glycol, and polyethylene glycol), oil (such as vegetable oil, e.g., peanut oil, corn oil, sesame oil), and combinations thereof. In some embodiments, the pharmaceutical formulations in the liquid dosage forms are aqueous formulations. Suitable solvents or dispersion mediums for aqueous formulations include, but are not limited to, water, buffers (such as acidic buffers), salines (such as normal saline), buffered salines (such as PBS), and Ringer's solution.

### C. Pharmaceutical Acceptable Carriers

In some embodiments, the pharmaceutical formulations are prepared using a pharmaceutically acceptable carrier, which encapsulates, embeds, entraps, dissolves, disperses, absorbs, and/or binds to a compound or composition disclosed herein. The pharmaceutical acceptable carrier is composed of materials that are considered safe and can be administered to a subject without causing undesirable biological side effects or unwanted interactions. Preferably, the pharmaceutically acceptable carrier does not interfere with the effectiveness of the compound or composition in performing its function. The pharmaceutically acceptable carrier can be formed of biodegradable materials, non-biodegradable materials, or combinations thereof. One or more of the pharmaceutical acceptable excipients described above may be present in the pharmaceutical acceptable carrier.

In some embodiments, the pharmaceutical acceptable carrier is a controlled-release carrier, such as delayed-release carriers, sustained-release (extended-release) carriers, and pulsatile-release carriers.

In some embodiments, the pharmaceutical acceptable carrier is pH-sensitive or pH-responsive. In some forms, the pharmaceutical acceptable carrier can decompose or degrade in a certain pH range. In some forms, the pharmaceutical acceptable carrier can experience a structural change when experiencing a change in the pH.

Exemplary pharmaceutical acceptable carriers include, but are not limited to: nanoparticles, microparticles, and combinations thereof; liposomes; hydrogels; polymer matrices; and solvent systems.

In some embodiments, the pharmaceutical acceptable carrier is nanoparticles, microparticles, or a combination thereof. In some embodiments, the compound or composition is embedded in the matrix formed by the materials of the nanoparticles, microparticles, or combination thereof.

The nanoparticles, microparticles, or combination thereof can be biodegradable, and optionally are capable of biodegrading at a controlled rate for delivery of the compound or composition. The nanoparticles, microparticles, or combination thereof can be made of a variety of materials. Both inorganic and organic materials can be used. Both polymeric and non-polymeric materials can be used.

For example, the nanoparticles, microparticles, or combination thereof are formed of one or more biocompatible polymers. In some forms, the biocompatible polymers are biodegradable. In some forms, the biocompatible polymers are non-biodegradable. In some forms, the nanoparticles, microparticles, or combination thereof are formed of a mixture of biodegradable and non-biodegradable polymers. The polymers used to form the nanoparticles, microparticles, or combination thereof may be tailored to optimize different characteristics of the nanoparticles, microparticles, or combination thereof, including: (i) interactions between the active ingredient and the polymer to provide stabilization of the active ingredient and retention of activity upon delivery; (ii) rate of polymer degradation and, thereby, rate of release; (iii) surface characteristics and targeting capabilities; and (iv) particle porosity.

Exemplary polymers include, but are not limited to, polymers prepared from lactones (such as poly(caprolactone)(PCL)), polyhydroxy acids and copolymers thereof (such as poly(lactic acid) (PLA), poly(glycolic acid)(PGA), and poly(lactic acid-co-glycolic acid)(PLGA)), polyalkyl cyanoacralate, polyurethanes, polyamino acids (such as poly-L-lysine (PLL), poly(valeric acid), poly-L-glutamic acid), hydroxypropyl methacrylate (HPMA), polyanhydrides, and polyorthoesters, poly(ester amides), polyamides, poly(ester ethers), polycarbonates, ethylene vinyl acetate polymer (EVA), polyvinyl alcohols (PVA), polyvinyl ethers, polyvinyl esters (such as poly(vinyl acetate)), polyvinyl halides (such as poly(vinyl chloride)(PVC)), polyvinylpyrrolidone, polysiloxanes, polystyrene (PS), celluloses and derivatized celluloses (such as alkyl celluloses, hydroxyalkyl celluloses, cellulose ethers, cellulose esters, nitro celluloses, hydroxypropylcellulose, and carboxymethylcellulose), polymers of acrylic acids (such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), and poly(octadecyl acrylate)), polydioxanone, polyhydroxyalkanoates, polypropylene fumarate, polyoxymethylene, poloxamers, poly(butyric acid), polyphosphazenes, polysaccharides, polypeptides, and blends thereof.

In some embodiments, the one or more biocompatible polymers forming the nanoparticles, microparticles, or combination thereof include an FDA-approved biodegradable polymer such as polyhydroxy acids (e.g., PLA, PGA, and PLGA), polyanhydrides, and polyhydroxyalkanoate (e.g., poly(3-butyrate) and poly(4-butyrate)).

Materials other than polymers may be used to form the nanoparticles, microparticles, or combination thereof. Suitable materials include surfactants. The use of surfactants in the nanoparticles, microparticles, or combination thereof may improve surface properties by, for example, reducing particle-particle interactions, and render the surface of the particles less adhesive. Both naturally occurring surfactants and synthetic surfactants can be incorporated into the nanoparticles, microparticles, or combination thereof. Exemplary surfactants include, but are not limited to, phosphoglycerides such as phosphatidylcholines (e.g., L-α-phosphatidylcholine dipalmitoyl), diphosphatidyl glycerol, hexadecanol, fatty alcohols, polyoxyethylene-9-lauryl ether, fatty acids such as palmitic acid and oleic acid, sorbitan trioleate, glycocholate, surfactin, poloxomers, sorbitan fatty acid esters such as sorbitan trioleate, tyloxapol, and phospholipids.

The nanoparticles, microparticles, or combination thereof may contain a plurality of layers. The layers can have similar or different release kinetic profiles for the active ingredient. For example, the nanoparticles, microparticles, or combination thereof can have a controlled-release core surrounded by one or more additional layers. The one or more additional layers can include an instant-release layer, preferably on the surface of the nanoparticles, microparticles, or combination thereof. The instant-release layer can provide a bolus of the active ingredient shortly after administration.

The composition and structure of the nanoparticles, microparticles, or combination thereof can be selected such that the nanoparticles, microparticles, or combination thereof are pH-sensitive or pH-responsive. In some embodiments, the nanoparticles, microparticles, or combination thereof are formed of one or more pH-sensitive or pH-responsive polymers such as the enteric polymers commercially available under the tradename EUDRAGIT®, as described above. Depending on the particle materials, the decomposition/degradation or structural change of the nanoparticles, microparticles, or combination thereof may occur at different locations of the gastrointestinal tract.

In some embodiments, the particle materials are selected such that the nanoparticles, microparticles, or combination thereof can survive exposure to gastric acid and release the active ingredient in the intestines after oral administration.

### D. Controlled Release

In some embodiments, the pharmaceutical formulations can be controlled-release formulations. Examples of controlled-release formulations include extended-release formulations, delayed-release formulations, and pulsatile-release formulations.

### 1. Extended Release

In some embodiments, the extended-release formulations are prepared as diffusion or osmotic systems, for example, as described in “Remington—The science and practice of pharmacy” (20th Ed., Lippincott Williams & Wilkins, 2000).

A diffusion system is typically in the form of a matrix, generally prepared by combining the active ingredient with a slowly dissolving, pharmaceutically acceptable carrier, optionally in a tablet form. Suitable materials used in the preparation of the matrix include plastics, hydrophilic polymers, and fatty compounds. Suitable plastics include, but are not limited to, acrylic polymer, methyl acrylate-methyl methacrylate copolymer, polyvinyl chloride, and polyethylene. Suitable hydrophilic polymers include, but are not limited to, cellulosic polymers such as methyl ethyl cellulose, hydroxyalkylcelluloses (such as hydroxypropylcellulose and hydroxypropylmethylcellulose), sodium carboxymethylcellulose, CARBOPOL® 934, polyethylene oxides, and combinations thereof. Suitable fatty compounds include, but are not limited to, various waxes such as carnauba wax and glyceryl tristearate, wax-type substances such as hydrogenated castor oil and hydrogenated vegetable oil, and combinations thereof.

In some embodiments, the plastic is a pharmaceutically acceptable acrylic polymer. In some embodiments, the pharmaceutically acceptable acrylic polymer is chosen from acrylic acid and methacrylic acid copolymers, methyl methacrylate copolymers, ethoxyethyl methacrylate copolymers, cyanoethyl methacrylate copolymers, aminoalkyl methacrylate copolymers, poly(acrylic acid), poly(methacrylic acid), methacrylic acid alkylamine copolymers, poly(methyl methacrylate), poly(methacrylic acid), polymethacrylate, polyacrylamide, poly(methacrylic acid anhydride), and glycidyl methacrylate copolymers.

In some embodiments, the pharmaceutically acceptable acrylic polymer can be an ammonio methacrylate copolymer. Ammonio methacrylate copolymers are well known in the art and are described as fully polymerized copolymers of acrylic and methacrylic acid esters with a low content of quaternary ammonium groups.

In some embodiments, the pharmaceutically acceptable acrylic polymer is an acrylic resin lacquer such as those commercially available under the tradename EUDRAGIT®. In some embodiments, the pharmaceutically acceptable acrylic polymer contains a mixture of two acrylic resin lacquers, EUDRAGIT® RL (such as EUDRAGIT® RL30D) and EUDRAGIT® RS (such as EUDRAGIT® RS30D). EUDRAGIT® RL30D and EUDRAGIT® RS30D are copolymers of acrylic and methacrylic acid esters with a low content of quaternary ammonium groups, the molar ratio of ammonium groups to the remaining neutral methacrylic esters being 1:20 in EUDRAGIT® RL30D and 1:40 in EUDRAGIT® RS30D. The code designations RL (high permeability) and RS (low permeability) refer to the permeability properties of these polymers. EUDRAGIT® RL/RS mixtures are insoluble in water and in digestive fluids. However, multi-particulate systems formed to include the same are swellable and permeable in aqueous solutions and digestive fluids. The EUDRAGIT® RL/RS mixtures may be prepared in any desired ratio in order to ultimately obtain a sustained-release formulation having a desirable release profile. Suitable sustained-release, multi-particulate systems may be obtained, for instance, from 90% EUDRAGIT® RL+10% EUDRAGIT® RS, to 50% EUDRAGIT® RL+50% EUDRAGIT® RS, and to 10% EUDRAGIT® RL+90% EUDRAGIT® RS. In some embodiments, the pharmaceutically acceptable acrylic polymer can also be or include other acrylic resin lacquers, such as EUDRAGIT® S-100, EUDRAGIT® L-100, and mixtures thereof.

Matrices with different release mechanisms or profiles can be combined in a final dosage form containing single or multiple units. Examples of multiple units include, but are not limited to, multilayer tablets and capsules containing beads, granules, and/or particles of the active ingredient. An immediate release portion can be added to the extended-release system by means of either applying an immediate release layer on top of the extended-release core using a coating or compression process or in a multiple unit system such as a capsule containing both extended- and immediate-release beads.

Extended-release tablets containing one or more of the hydrophilic polymers can be prepared by techniques commonly known in the art such as direct compression, wet granulation, and dry granulation.

Extended-release tablets containing one or more of the fatty compounds can be prepared using methods known in the art such as direct blend methods, congealing methods, and aqueous dispersion methods. In the congealing methods, the active ingredient is mixed with the fatty compound(s) and congealed.

Alternatively, the extended-release formulations can be prepared using osmotic systems or by applying a semi-permeable coating to a solid dosage form. In the latter case, the desired release profile can be achieved by combining low permeable and high permeable coating materials in suitable proportions.

### 2. Delayed Release

Delayed-release formulations can be prepared by coating a solid dosage form with a coating. In some embodiments, the coating is insoluble and impermeable in the acidic environment of the stomach and becomes soluble or permeable in the less acidic environment of the intestines and/or the lower GI tract. In some embodiments, the solid dosage form is a tablet for incorporation into a capsule, a tablet for use as an inner core in a “coated-core” dosage form, or a plurality of beads, granules, and/or particles containing the active ingredient, for incorporation into either a tablet or capsule.

Suitable coating materials may be bioerodible polymers, gradually hydrolysable polymers, gradually water-dissolvable polymers, and enzymatically degradable polymers. In some embodiments, the coating material is or contains enteric polymers. Combinations of different coating materials may also be used. Multilayer coatings using different coating materials may also be applied.

The coating may also contain one or more additives, such as plasticizers as described above (optionally representing about 10 wt % to 50 wt % relative to the dry weight of the coating), colorants as described above, stabilizers as described above, glidants as described above, etc.

### 3. Pulsatile Release

Pulsatile-release formulations release a plurality of doses of the active ingredient at spaced-apart time intervals. Generally, upon administration, such as oral administration, of the pulsatile-release formulations, release of the initial dose is substantially immediate, e.g., the first release “pulse” occurs within about three hours, two hours, or one hour of administration. This initial pulse may be followed by a first time-interval (lag time) during which very little or no active ingredient is released from the formulations, after which a second dose may be released. Similarly, a second lag time (nearly release-free interval) between the second and third release pulses may be designed.

The duration of the lag times will vary depending on the formulation design, especially on the length of the dosing interval, e.g., a twice daily dosing profile, a three-time daily dosing profile, etc.

For pulsatile-release formulations providing a twice daily dosage profile, they deliver two release pulses of the active ingredient. In some embodiments, the one nearly release-free interval between the first and second release pulses may have a duration of between 3 hours and 14 hours.

For pulsatile-release formulations providing a three daily dosage profile, they deliver three release pulses of the active ingredient. In some embodiments, the two nearly release-free interval between two adjacent pulses may have a duration of between 2 hours and 8 hours.

In some embodiments, the pulsatile-release formulations contain a plurality of pharmaceutically acceptable carriers with different release kinetics.

In some embodiments, the pulsatile-release formulations contain a pharmaceutically acceptable carrier with a plurality of layers loaded with the active ingredient. In some embodiments, the layers may have different release kinetics. In some embodiments, the layers may be separated by a delayed-release coating. For example, the pulsatile-release formulations may have a first layer loaded with the active ingredient on the surface for the first release pulse and a second layer, e.g., a core loaded with the active ingredient, for the second release pulse; the second layer may be surrounded by a delayed-release coating, which creates a lag time between the two release pulses.

In some embodiments, the pulsatile-release profile is achieved with formulations that are closed and optionally sealed capsules housing at least two “dosage units” wherein each dosage unit within the capsules provides a different release profile. In some embodiments, at least one of the dosage units is a delayed-release dosage unit. Control of the delayed-release dosage unit(s) may be accomplished by a controlled-release polymer coating on the dosage unit(s) or by incorporation of the active ingredient in a controlled-release polymer matrix. In some embodiments, each dosage unit may comprise a compressed or molded tablet, wherein each tablet within the capsule provides a different release profile.

### E. Exemplary Formulations for Different Routes of Administration

A subject suffering from a condition, disorder, or disease as described herein, can be treated by either targeted or systemic administration, via oral, inhalation, topical, trans- or sub-mucosal, subcutaneous, intramuscular, intravenous, or transdermal administration of a pharmaceutical formulation containing a compound or composition described herein. In some embodiments, the pharmaceutical formulation is suitable for oral administration. In some embodiments, the pharmaceutical formulation is suitable for subcutaneous, intravenous, or intramuscular administration. In some embodiments, the pharmaceutical formulation is suitable for inhalation or intranasal administration. In some embodiments, the pharmaceutical formulation is suitable for transdermal or topical administration.

In some embodiments, the pharmaceutical formulation is an oral pharmaceutical formulation. In some embodiments, the active ingredient may be incorporated with one or more pharmaceutically acceptable excipients as described above and used in the form of tablets, pills, caplets, or capsules. For example, the corresponding oral pharmaceutical formulation may contain one or more of the following pharmaceutically acceptable excipients or those of a similar nature: a binder as described above, a disintegrant as described above, a lubricant as described above, a glidant as described above, a sweetening agent (such as sucrose and saccharin), and a flavoring agent (such as methyl salicylate and fruit flavorings). In some embodiments, when the oral pharmaceutical formulation is in the form of capsules, it may contain, in addition to the material(s) listed above, a liquid carrier (such as a fatty oil). In some embodiments, when the oral pharmaceutical formulation is in the form of capsules, each capsule may contain a plurality of beads, granules, and/or particles of the active ingredient. In some embodiments, the oral pharmaceutical formulation may contain one or more other materials which modify the physical form or one or more pharmaceutical properties of the dosage unit, for example, coatings of polysaccharides, shellac, or enteric polymers as described in previous sections.

In some embodiments, the oral pharmaceutical formulation can be in the form of an elixir, suspension, syrup, wafer, chewing gum or the like. A syrup may contain, in addition to the active ingredient, one or more sweetening agents (such as sucrose and saccharine), one or more flavoring agents, one or more preservatives, and/or one or more dyes or colorings.

In some embodiments, the pharmaceutical formulation is a subcutaneous, intramuscular, or intravenous pharmaceutical formulation. In some embodiments, the subcutaneous, intramuscular, or intravenous pharmaceutical formulation can be enclosed in an ampoule, syringe, or a single or multiple dose vial made of glass or plastic. In some embodiments, the subcutaneous, intramuscular, or intravenous pharmaceutical formulation contains a liquid pharmaceutically acceptable carrier for the active ingredient. Suitable liquid pharmaceutically acceptable carriers include, but are not limited to, water, buffer, saline, buffered saline (such as PBS), and combinations thereof.

In some embodiments, the pharmaceutical formulation is a topical pharmaceutical formulation. Suitable forms of the topical pharmaceutical formulation include lotions, suspensions, ointments, creams, gels, tinctures, sprays, powders, pastes, slow-release transdermal patches, and suppositories for application to rectal, vaginal, nasal, or oral mucosa. In some embodiments, thickening agents, emollients (such as mineral oil, lanolin and its derivatives, and squalene), humectants (such as sorbitol), and/or stabilizers can be used to prepare the topical pharmaceutical formulations. Examples of thickening agents include petrolatum, beeswax, xanthan gum, and polyethylene.

In some embodiments, the pharmaceutical formulation is an intranasal pharmaceutical formulation. In some embodiments, the intranasal pharmaceutical formulation is in the form of an aqueous suspension, which can be optionally placed in a pump spray bottle. Other than water, the aqueous suspension may contain one or more pharmaceutically acceptable excipients, such as suspending agents (e.g., microcrystalline cellulose, sodium carboxymethylcellulose, hydroxypropyl-methyl cellulose), humectants (e.g., glycerol, propylene glycol), acids, bases, and/or pH-buffering agents for adjusting the pH (e.g., citric acid, sodium citrate, phosphoric acid, sodium phosphate, and combinations thereof), surfactants (e.g., polysorbate 80), and preservatives (e.g., benzalkonium chloride, phenylethyl alcohol, potassium sorbate).

In some embodiments, the pharmaceutical formulation is an inhalation pharmaceutical formulation. In some embodiments, the inhalation pharmaceutical formulation may be in the form of an aerosol suspension, a dry powder, or a liquid suspension. The inhalation pharmaceutical formulation may be prepared for delivery as a nasal spray or an inhaler, such as a metered dose inhaler (MDI). In some embodiments, MDIs can deliver aerosolized particles suspended in chlorofluorocarbon propellants such as CFC-11 and CFC-12, or non-chlorofluorocarbons or alternate propellants such as fluorocarbons (e.g., HFC-134A, HFC-227), with or without surfactants or suitable bridging agents. Dry-powder inhalers can also be used, either breath activated or delivered by pressure.

In some embodiments, the active ingredient is prepared with a pharmaceutically acceptable carrier that will protect it against rapid degradation or elimination from the body of the subject after administration, such as the controlled-release formulations described in previous sections.

### V. METHODS OF USING

Disclosed are methods of treating a condition, disorder, or disease in a subject in need thereof. The methods include administering an effective amount of a compound, composition, or pharmaceutical formulation disclosed herein to the subject.

The compound, composition, or pharmaceutical formulation can be administered in a variety of manners, depending on whether local or systemic administration is desired. In some embodiments, the compound, composition, or pharmaceutical formulation is directly administered to a specific bodily location of the subject, e.g., topical administration and intranasal administration. In some embodiments, the compound, composition, or pharmaceutical formulation is administered in a systemic manner, such as enteral administration (e.g., oral administration) and parenteral administration (e.g., injection, infusion, and implantation). Exemplary administration routes include oral administration, intravenous administration such as intravenous injection or infusion, intramuscular administration such as intramuscular injection, intranasal administration, and topical administration. In some embodiments, the compound, composition, or pharmaceutical formulation is administered orally. In some embodiments, the compound, composition, or pharmaceutical formulation is administered intravenously. In some embodiments, the compound, composition, or pharmaceutical formulation is administered intramuscularly.

In some embodiments, the subject is a human. In some embodiments, the subject is a non-human animal, such as domestic pets, livestock and farm animals, and zoo animals. In some embodiments, the non-human animal may be a non-human primate.

### A. Indications

The utility of the compounds, compositions, and pharmaceutical formulations of this disclosure may be applied to conditions, disorders, and diseases that can lead to neurological damage, neuronal loss, cerebral edema, and/or neuroinflammation.

Exemplary conditions, disorders, and diseases that can be treated by the disclosed compounds, compositions, and formulations include, but are not limited to, stroke, subarachnoid hemorrhage, cerebral ischemia, cerebral vasospasm, hypoxia, CNS injury (such as head injury and spinal cord injury), concussion, traumatic brain injury, depression, postpartum depression, epilepsy, seizure disorder, and neurodegenerative disease.

In some embodiments, the condition, disorder, or disease is chosen from stroke, subarachnoid hemorrhage, traumatic brain injury, concussion, dementia, Alzheimer's diseases, epilepsy, seizure disorder, depression, and postpartum depression.

In some embodiments, the condition, disorder, or disease is stroke. In some embodiments, the compound, composition, or pharmaceutical formulation is used to treat or prevent stroke-associated damages. In some embodiments, the compound, composition, or pharmaceutical formulation is administered under emergency care for stroke, for maintenance treatment of stroke, and/or for rehabilitation of stroke.

In some embodiments, the condition, disorder, or disease is subarachnoid hemorrhage (SAH). In some embodiments, the compound, composition, or pharmaceutical formulation is used to treat or prevent SAH-associated damages. In some embodiments, the compound, composition or pharmaceutical formulation is administered under emergency care for a SAH, for maintenance treatment of SAH, and/or for rehabilitation of SAH.

SAH refers to an abnormal condition in which blood collects beneath the arachnoid mater, a membrane that covers the brain. This area, called the subarachnoid space, normally contains cerebrospinal fluid. The accumulation of blood in the subarachnoid space, and the vasospasm of the vessels which results from it, can lead to stroke, seizures, and other complications. SAH can be spontaneous or caused by a head injury. The compound, composition, or pharmaceutical formulation can be used to treat a subject experiencing SAH. For example, the compound, composition, or pharmaceutical formulation can be used to prevent or limit one or more of the toxic effects of SAH, including, for example, stroke and ischemia that can result from SAH. Alternatively, the compound, composition, or pharmaceutical formulation can be used to treat a subject with traumatic subarachnoid hemorrhage caused by a head injury.

In some embodiments, the condition, disorder, or disease is cerebral ischemia. In some embodiments, the compound, composition, or pharmaceutical formulation is used to treat or prevent cerebral ischemia-associated damages. In some embodiments, the compound, composition, or pharmaceutical formulation is administered under emergency care for a cerebral ischemia event, for maintenance treatment of cerebral ischemia, and/or for rehabilitation of cerebral ischemia. In some embodiments, the cerebral ischemia is caused by traumatic brain injury, coronary artery bypass graft, carotid angioplasty, or neonatal ischemia following hypothermic circulatory arrest.

In some embodiments, the condition, disorder, or disease is cerebral vasospasm. In some embodiments, the cerebral vasospasm is caused or induced by SAH.

In some embodiments, the condition, disorder, or disease is depression or postpartum depression. In some embodiments, the depression is treatment-resistant depression.

In some embodiments, the condition, disorder, or disease is a neurodegenerative disease such as dementia and Alzheimer's disease. In some embodiments, the compound, composition, or pharmaceutical formulation is used to reduce one or more symptoms of the neurodegenerative disease. In some embodiments, the compound, composition, or pharmaceutical formulation is used to provide cognitive enhancement to the subject that suffers from the neurodegenerative disease. In some embodiments, the neurodegenerative disease is Alzheimer's disease. In some embodiments, the neurodegenerative disease is dementia. In some embodiments, the dementia is AIDS-induced dementia.

In some embodiments, the condition, disorder, or disease is epilepsy or seizure disorder. In some embodiments, the epilepsy or seizure disorder may be selected from epilepsies that are inadequately controlled by existing medications (i.e., treatment-resistant epilepsy), infantile spasms, and epilepsies or seizure disorders caused by a rare disease or genetic condition (e.g., genetic mutation) that produces epilepsies, seizures, spasms, abnormally hypersynchronous brain activity, and/or other conditions associated with enhanced neuronal synchrony. In some embodiments, the subject may be a pediatric patient suffering from the epilepsy or seizure disorder. In some embodiments, the subject may be an adult patient suffering from the epilepsy or seizure disorder. In some embodiments, the compound, composition, or pharmaceutical formulation is used to reduce the severity and/or intensity of the epilepsy or seizure disorder. In some embodiments, the compound, composition, or pharmaceutical formulation is used to reduce the frequency of the epilepsy or seizure disorder.

In some embodiments, the condition, disorder, or disease is hypoxia. In some embodiments, the compound, composition, or pharmaceutical formulation is used to treat or prevent hypoxia-associated damages. In some embodiments, the compound, composition, or pharmaceutical formulation is administered under emergency care for a hypoxia event, for maintenance treatment of hypoxia, and/or for rehabilitation of hypoxia. In some embodiments, the hypoxia is induced by respiratory insufficiency, prolonged use of ventilator, or both. In some embodiments, the respiratory insufficiency, prolonged use of ventilator, or both is associated with COVID-19, including hospitalization caused by COVID-19.

### B. Dosing and Administration

In some embodiments, the compound, composition, or pharmaceutical formulation is administered for a sufficient time period to alleviate one or more undesired symptoms and/or one or more clinical signs associated with the condition, disorder, or disease being treated. In some embodiments, the compound, composition, or pharmaceutical formulation is administered less than three times daily. In some embodiments, the compound, composition, or pharmaceutical formulation is administered once or twice daily. In some embodiments, the compound, composition, or pharmaceutical formulation is administered once daily. In some embodiments, the compound, composition, or pharmaceutical formulation is administered in a single oral dosage once a day. In some embodiments, the compound, composition, or pharmaceutical formulation is administered in a single intravenous dosage once a day. In some embodiments, the compound, composition, or pharmaceutical formulation is administered in a single intramuscular dosage once a day.

In cases of acute brain injuries, such as stroke, concussion, and traumatic brain injury, the compound, composition, or pharmaceutical formulation may be administered under emergency care via intramuscular injection to minimize the onset of action.

In cases of chronic or non-acute illnesses, such as depression and postpartum depression, the compound, composition, or pharmaceutical formulation may be administered via oral administration or intravenous infusion.

### EXAMPLES

The examples below describe studies to synthesize and evaluate prodrugs of neurosteroid analogs.

General information for synthetic chemistry: All chemicals were purchased from commercial vendors and used without further purification unless stated otherwise. Dichloromethane (DCM), toluene, dimethylformamide (DMF), tetrahydrofuran (THF), ether, and triethylamine (TEA) were purchased anhydrous in septum-sealed bottles from Sigma Aldrich. All reactions were conducted using oven- or flame-dried glassware under an inert atmosphere of argon unless noted otherwise. Thin layer chromatography (TLC) was utilized to monitor reaction progress using silica gel 60 F254 aluminum-backed plates. TLC spots were visualized with UV light, KMnO₄, PMA, or ninhydrin stains. Flash chromatography was performed using a Teledyne Isco CombiFlash Rf® system using RediSep® Rf silica gel disposable flash columns (60 Å pore size, 40-60 μm particle size). NMR spectra were acquired using a 400 MHz Varian INOVA or a 600 MHz Bruker Avance III NMR spectrometer. Chemical shifts are reported in δ ppm and referenced using residual solvent peaks (CDCl₃ or TMS). Rotamer signals are denoted with *. High resolution mass spectrometry (HRMS) was performed on a Thermo Exactive Plus Orbitrap Mass Spectrometer using APCI or ESI ionization methods.

### Example 1. Preparation of tert-butyl(2-((2-((tert-butyldimethylsilyl)oxy)ethyl)amino)ethyl)(methyl)carbamate (6)

tert-Butyl (2-((2-((tert-butyldimethylsilyl)oxy)ethyl)amino)ethyl)(methyl)carbamate (6) was prepared according to Scheme 1.

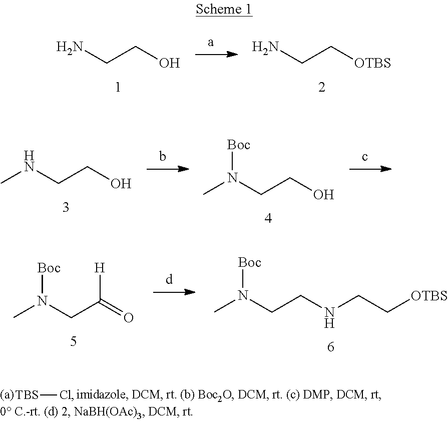

### A. Preparation of 2-((tert-butyldimethylsilyl)oxy)ethan-1-amine (2)

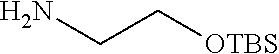 [SMILES: CC(C)(C)[Si](C)(C)OCCN]

An oven-dried 250 mL 2-neck round bottom flask equipped with a stirrer bar was charged with 2-aminoethan-1-ol (1)(2.0 g, 32.7 mmol), imidazole (4.46 g, 65.49 mmol) and dissolved in anhydrous DCM (40 mL). A solution of TBS-Cl (5.43 g, 36.02 mmol) in anhydrous DCM (15 mL) was then added dropwise at room temperature and the reaction mixture was stirred for 3 h. The reaction was then diluted with water (20 mL) and extracted with DCM (2×50 mL). The combined organic layer was then washed with brine, dried over anhydrous magnesium sulfate, filtered, and concentrated in vacuo to afford the product 2-((tert-butyldimethylsilyl)oxy)ethan-1-amine (2) (5.020 g, 28.663 mmol, 87%) as a clear yellowish oil. No further purification was required. ¹H and ¹³C NMR spectra correspond to the literature (Inman and Moody, J. Org. Chem., 2010, 75 (17), 6023-6026).

### B. Preparation of tert-butyl(2-hydroxyethyl)(methyl)carbamate (4)

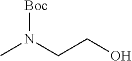

To an oven-dried 150 mL 2-neck round bottom flask, equipped with stirrer bar, was added 2-(methylamino) ethan-1-ol (3)(1.0 g, 13.3 mmol) and anhydrous DCM (40 mL) under argon, followed by the drop-wise addition of Boc₂O (3.05 g, 13.97 mmol). The reaction mixture was stirred at room temperature for 2 h. The reaction was then diluted with DCM (40 mL), extracted with brine and dried over anhydrous magnesium sulfate. After filtration, the product tert-butyl(2-hydroxyethyl)(methyl)carbamate (4)(2.300 g, 13.126 mmol, quant.) was concentrated in vacuo and no further purification was required. ¹H and ¹³C NMR spectra correspond to the literature (Söveges, et al., Org. Biomol. Chem., 2016, 14 (25), 6071-6078).

### C. Preparation of tert-butyl methyl(2-oxoethyl)carbamate (5)

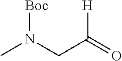

An oven-dried 150 mL 2-neck round bottom flask, equipped with a stirrer bar, was charged with DMP (3.388 g, 7.988 mmol) and 40 mL anhydrous DCM under an argon atmosphere. The reaction mixture was cooled to 0° C. in an ice-bath and treated with a solution of tert-butyl(2-hydroxyethyl)(methyl)carbamate (4)(2.0 g, 32.7 mmol) in 15 mL anhydrous DCM. The reaction was stirred at 0° C. for 30 min and then at room temperature for 2 h. Upon completion, saturated sodium thiosulfate solution (75 mL) and saturated sodium bicarbonate solution (75 mL) were added to the reaction mixture and vigorously stirred for an additional 30 min. Finally, the organic layer was collected and extracted with saturated sodium bicarbonate solution (50 mL) and brine (50 mL). The organic layer was dried over anhydrous magnesium sulfate, filtered, and concentrated in vacuo. Flash chromatography (10-40% ethyl acetate in hexanes) afforded tert-butyl methyl(2-oxoethyl)carbamate (5)(1.1 g, 6.4 mmol, 95%) as a clear oil. ¹H and ¹³C NMR spectra correspond to the literature (Blaney, et al., Tetrahedron, 2002, 58 (9), 1719-1737).

### D. Preparation of tert-butyl(2-((2-((tert-butyldimethylsilyl)oxy)ethyl)amino)ethyl)(methyl)carbamate (6)

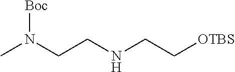 [SMILES: CN(CCNCCO[Si](C)(C)C(C)(C)C)C(=O)OC(C)(C)C]

An oven-dried 150 mL 2-neck round bottom flask was charged with a stirrer bar and tert-butyl methyl(2-oxoethyl)carbamate (5)(1.0 g, 5.7 mmol) in anhydrous DCM (40 mL) under an argon atmosphere. 2-((tert-Butyldimethylsilyl)oxy)ethan-1-amine (2)(1.32 g, 7.51 mmol) was then added dropwise and the reaction mixture was stirred for 30 min at room temperature. Sodium triacetoxyborohydride (1.84 g, 8.66 mmol) was then added in one portion and the mixture was further stirred overnight. The reaction was quenched with saturated sodium bicarbonate (30 mL) and extracted with DCM (50 mL). The organic layer was then washed with brine, dried over anhydrous magnesium sulfate, filtered, and concentrated in vacuo. Flash chromatography (0-10% MeOH/DCM) afforded (2-((2-((tert-butyldimethylsilyl)oxy)ethyl)amino)ethyl)(methyl)carbamate (6)(1.02 g, 3.07 mmol, 53%) as a clear oil. ¹H spectrum correspond to the literature (Yamaguchi-Sasaki, et al., Bioorg. Med. Chem., 2020, 28 (24), 115818).

### Example 2. Preparation of tert-butyl(2-((tert-butyldimethylsilyl)oxy)ethyl)(2-(methylamino)ethyl)carbamate (11a) and tert-butyl(2-fluoroethyl)(2-(methylamino)ethyl)carbamate (11b)

tert-Butyl (2-((tert-butyldimethylsilyl)oxy)ethyl)(2-(methylamino)ethyl)carbamate (11a) and tert-butyl(2-fluoroethyl)(2-(methylamino)ethyl)carbamate (11b) were prepared according to Scheme 2.

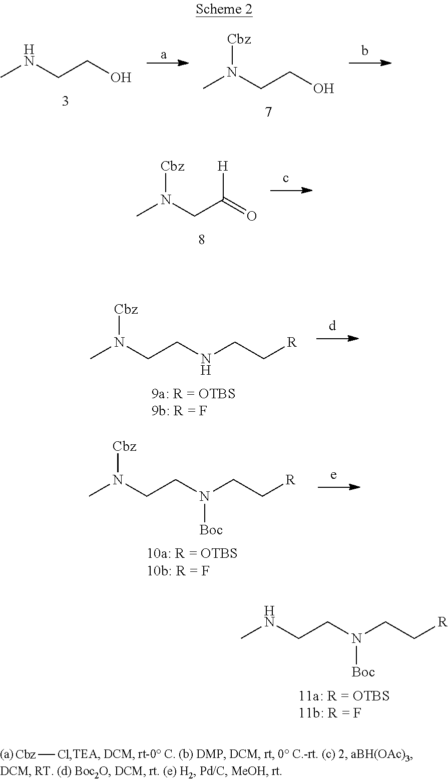

### A. Preparation of benzyl (2-hydroxyethyl)(methyl)carbamate (7)

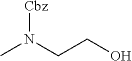

To an oven-dried 2-neck round bottom flask was added 2-(methylamino) ethan-1-ol (3) (500 mg, 6.66 mmol) and anhydrous DCM (35 mL). The solution was then cooled to 0° C. and treated with benzyl chloroformate (1.25 g, 7.32 mmol), followed by the drop-wise addition of TEA (1.1 mL, 7.99 mmol). The reaction mixture was kept in the ice bath and stirred overnight. The mixture was then extracted with 1M HCl_{(aq)} (20 mL). The collected organic layer was further extracted with saturated sodium bicarbonate solution (20 mL). The organic layer was finally washed with brine, dried over anhydrous magnesium sulfate, filtered, and concentrated in vacuo. Flash chromatography (20-70% ethyl acetate/hexane) yielded benzyl (2-hydroxyethyl)(methyl)carbamate (7)(1.19 g, 5.67 mmol, 86%) as a clear oil. ¹H and ¹³C NMR spectra correspond to the literature (Savicheva, et al., Angew. Chem. Int. Ed., 2020, 59 (14), 5505-5509).

### B. Preparation of benzyl methyl(2-oxoethyl)carbamate (8)

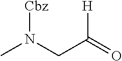

An oven-dried 150 mL 2-neck round bottom flask, equipped with a stirrer bar, was charged with DMP (1.217 g, 1.217 mmol) and anhydrous DCM (20 mL) under an argon atmosphere. The reaction mixture was cooled to 0° C. in an ice-bath and treated with a solution of benzyl (2-hydroxyethyl)(methyl)carbamate (7)(500 mg, 2.389 mmol) in anhydrous DCM (7 mL). The reaction was stirred at 0° C. for 30 min and then at room temperature for 4 h. Upon completion, 30 mL sodium thiosulfate and saturated sodium bicarbonate solution (30 mL) was added to the reaction mixture and vigorously stirred for a further 30 min. Finally, the organic layer was collected and extracted with saturated sodium bicarbonate (30 mL) and then brine (30 mL). The organic layer was dried over anhydrous magnesium sulfate, filtered, and concentrated in vacuo. Flash chromatography (10-40% ethyl acetate in hexanes) afforded benzyl methyl(2-oxoethyl)carbamate (8)(490 mg, 2.353 mmol, quant.) as a clear oil. ¹H NMR spectrum correspond to the literature (Martin, et al., J. Org. Chem., 1987, 52 (10), 1962-1972).

### C. Preparation of benzyl (2-((2-((tert-butyldimethylsilyl)oxy)ethyl)amino)ethyl)(methyl)carbamate (9a)

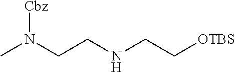 [SMILES: CN(CCNCCO[Si](C)(C)C(C)(C)C)C(=O)OCc1ccccc1]

An oven-dried 150 mL 2-neck round bottom flask was charged with a stirrer bar and benzyl methyl(2-oxoethyl)carbamate (8)(939 mg, 4.51 mmol) in anhydrous DCM (30 mL) under an argon atmosphere. 2-((tert-butyldimethylsilyl)oxy)ethan-1-amine (2)(947 mg, 5.40 mmol) was then added dropwise and the reaction mixture was stirred for 30 min at room temperature. Sodium triacetoxyborohydride (1.434 g, 6.766 mmol) was then added in one portion and the mixture was further stirred overnight. The reaction was quenched with 20 mL sodium bicarbonate and extracted with 40 mL DCM. The organic layer was then washed with brine, dried over anhydrous magnesium sulfate, filtered, and concentrated in vacuo. Flash chromatography (0-10% MeOH/DCM) afforded benzyl (2-((2-((tert-butyldimethylsilyl)oxy)ethyl)amino)ethyl)(methyl)carbamate (9a) together with the disubstituted species (1.25 g) as an oil. The subsequent reaction was carried out using the crude mixture to ease purification.

### D. Preparation of benzyl (2-((2-fluoroethyl)amino)ethyl)(methyl)carbamate (9b)

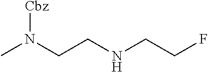 [SMILES: CN(CCNCCF)C(=O)OCc1ccccc1]

An oven-dried 150 mL 2-neck round bottom flask was charged with a stirrer bar, benzyl methyl(2-oxoethyl)carbamate (8)(850 mg, 4.08 mmol), and 2-fluoroethylamine hydrochloride (489 mg, 4.91 mmol) in anhydrous DCM (25 mL) under an argon atmosphere. TEA (0.68 mL, 4.90 mmol) was then added, and the reaction mixture was stirred for 1 h at room temperature. Sodium triacetoxyborohydride (1.297 g, 6.12 mmol) was then added in one portion and the mixture was further stirred overnight. The reaction was quenched with 20 mL sodium bicarbonate and extracted with 40 mL DCM. The organic layer was then washed with brine, dried over anhydrous magnesium sulfate, filtered, and concentrated in vacuo. Flash chromatography (0-10% MeOH/DCM) afforded benzyl (2-((2-fluoroethyl)amino)ethyl)(methyl)carbamate (9b) together with the disubstituted species (0.990 g) as an oil. The subsequent reaction was carried out using the crude mixture to ease purification.

### E. Preparation of benzyl (2-((tert-butoxycarbonyl)(2-((tert-butyldimethylsilyl)oxy)ethyl)amino)ethyl)(methyl)carbamate (10a)

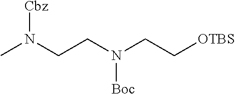 [SMILES: CN(CCN(CCO[Si](C)(C)C(C)(C)C)C(=O)OC(C)(C)C)C(=O)OCc1ccccc1]

To an oven-dried 150 mL 2-neck round bottom flask, equipped with a stirrer bar, was added benzyl (2-((2-((tert-butyldimethylsilyl)oxy)ethyl)amino)ethyl)(methyl)carbamate (9a)(1.250 g, 3.415 mmol) and anhydrous DCM (12 mL) under argon, followed by the dropwise addition of Boc₂O (0.79, 3.415 mmol). The reaction mixture was stirred at room temperature for 2 h. The reaction was then diluted with DCM (20 mL), extracted with brine, dried over anhydrous magnesium sulfate, filtered, and reduced in vacuo. Flash chromatography (0-25% ethyl acetate in hexanes) afforded the product benzyl (2-((tert-butoxycarbonyl)(2-((tert-butyldimethylsilyl)oxy)ethyl)amino)ethyl)(methyl)carbamate (10a)(930 mg, 1.99 mmol, 58% over two steps) as a clear oil. ¹H NMR (400 MHZ, CDCl₃) δ 7.39-7.27 (m, 5H), 5.11 (s, 2H), 3.76-3.56 (m, 2H), 3.47-3.11 (m, 6H), 2.98-2.91 (m, 3H), 1.45 (s, 9H), 1.43* (s, 9H), 0.87 (s, 9H), 0.04 (s, 6H), 0.02* (s, 6H); ¹³C NMR (101 MHZ, CDCl₃) δ 156.3, 155.6, 136.9, 128.6 (2C), 128.1 (2C), 127.9, 79.8, 77.4, 67.2, 61.71, 49.9, 46.9, 35.5, 28.6 (3C), 26.0 (3C), 18.37, −5.3 (2C). HRMS: (APCI+) [M+H]⁺ calc. for C₂₄H₄₃N₂O₅Si, 467.29358, observed, 467.29337.

### F. Preparation of benzyl (2-((tert-butoxycarbonyl)(2-fluoroethyl)amino)ethyl)(methyl)carbamate (10b)

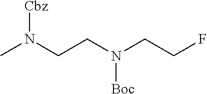 [SMILES: CN(CCN(CCF)C(=O)OC(C)(C)C)C(=O)OCc1ccccc1]

To an oven-dried 150 mL 2-neck round bottom flask, equipped with a stirrer bar, was added benzyl (2-((2-fluoroethyl)amino)ethyl)(methyl)carbamate (9b)(820 mg, 3.228 mmol) and anhydrous DCM (12 mL) under argon, followed by the dropwise addition of Boc₂O (0.74, 3.228 mmol). The reaction mixture was stirred at room temperature for 2 h. The reaction was then diluted with DCM (20 mL), extracted with brine, dried over anhydrous magnesium sulfate, filtered, and reduced in vacuo. Flash chromatography (0-40% ethyl acetate in hexanes) afforded the product benzyl (2-((tert-butoxycarbonyl)(2-fluoroethyl)amino)ethyl)(methyl)carbamate (10b)(555 mg, 1.57 mmol, 43% over two steps) as a clear oil. ¹H NMR (600 MHZ, CDCl₃) δ 7.38-7.27 (m, 5H), 5.12 (s, 2H), 4.61-4.29 (m, 2H), 3.57-3.24 (m, 6H), 2.95 (s, 3H), 1.45 (s, 9H), 1.43* (s, 9H). ¹³C NMR (101 MHZ, CDCl₃) δ 156.2, 155.4, 136.7, 128.6, 128.2, 127.9, 82.9, 80.3, 67.4, 48.4, 47.4, 46.3, 35.5, 28.4.

### G. Preparation of tert-butyl(2-((tert-butyldimethylsilyl)oxy)ethyl)(2-(methylamino)ethyl)carbamate (11a)

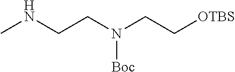 [SMILES: CNCCN(CCO[Si](C)(C)C(C)(C)C)C(=O)OC(C)(C)C]

An oven-dried 100 mL round bottom flask was charged with a stirrer bar, benzyl (2-((tert-butoxycarbonyl)(2-((tert-butyldimethylsilyl)oxy)ethyl)amino)ethyl)(methyl)carbamate (10a) (880 mg, 1.89 mmol), palladium on carbon (49 mg, 0.47 mmol) and methanol (20 mL) under an argon atmosphere. A T-joint bearing a hydrogen balloon and Schlenk line connection was affixed to the flask. The reaction flask was then briefly evacuated and filled with hydrogen (×2). The mixture was then allowed to stir at room temperature under hydrogen for 2 h. The mixture was then filtered over celite and washed with methanol (10 mL). The combined eluants were then concentrated in vacuo. Flash chromatography (2-15% MeOH/DCM) afforded tert-butyl(2-((tert-butyldimethylsilyl)oxy)ethyl)(2-(methylamino)ethyl)carbamate (11a)(605 mg, 1.82 mmol, 96%) as a clear oil. ¹H NMR (600 MHZ, CDCl₃) δ 3.76-3.65 (m, 2H), 3.44-3.26 (m, 4H), 2.80-2.69 (m, 2H), 2.45 (s, 3H), 2.06 (br s, 1H), 1.44 (s, 9H), 0.87 (s, 9H), 0.04 (s, 6H); ¹³C NMR (151 MHz, CDCl₃) δ 155.8, 79.7, 61.8, 50.6, 50.4, 48.3, 36.3, 28.6, 26.1, 18.4, −5.3. HRMS (APCI+) [M+H]⁺ calc. for C₁₆H₃₇N₂O₃Si, 333.2568, observed, 333.2565.

### H. Preparation of tert-butyl(2-fluoroethyl)(2-(methylamino)ethyl)carbamate (11b)

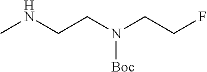

An oven-dried 100 mL round bottom flask was charged with a stirrer bar, benzyl (2-((tert-butoxycarbonyl)(2-fluoroethyl)amino)ethyl)(methyl)carbamate (10b)(350 mg, 0.99 mmol), palladium on carbon (26 mg, 0.25 mmol), and methanol (6 mL) under an argon atmosphere. A T-joint bearing a hydrogen balloon and Schlenk line connection was affixed to the flask. The reaction flask was then briefly evacuated and filled with hydrogen (×2). The mixture was stirred at room temperature under hydrogen for 2 h. The mixture was then filtered over celite and washed with methanol (5 mL). The combined eluants were concentrated in vacuo, affording the crude product tert-butyl(2-fluoroethyl)(2-(methylamino)ethyl)carbamate (11b)(211 mg, 0.958 mmol, 97%) as a clear oil. The crude product was carried forward to the next reaction without further purification.

### Example 3. Preparation of tert-butyl(S)-2-((methylamino)methyl)pyrrolidine-1-carboxylate (15a) and tert-butyl(S)-2-((methylamino)methyl)piperidine-1-carboxylate (15b)

tert-Butyl(S)-2-((methylamino)methyl)pyrrolidine-1-carboxylate (15a) and tert-butyl(S)-2-((methylamino)methyl)piperidine-1-carboxylate (15b) were prepared according to Scheme 3.

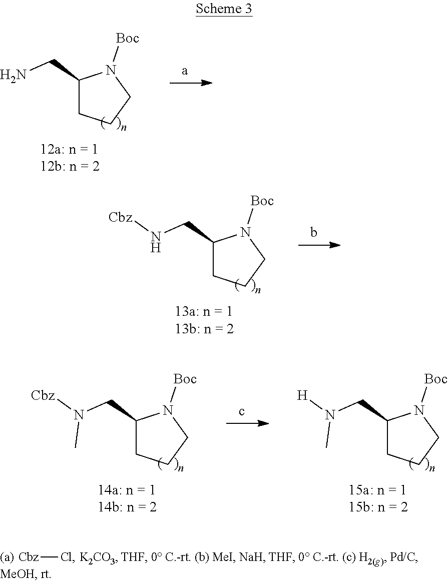

### A. Preparation of tert-butyl(S)-2-((((benzyloxy)carbonyl)amino)methyl)pyrrolidine-1-carboxylate (13a)

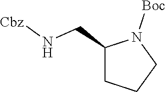 [SMILES: CC(C)(C)OC(=O)N1CCC[C@H]1CNC(=O)OCc1ccccc1]

An oven-dried 100 mL Schlenk tube was charged with a stirrer bar and tert-butyl(25)-2-(aminomethyl)pyrrolidine-1-carboxylate (12a)(1500.00 mg, 7.49 mmol) and placed under argon. Anhydrous THF (20 mL) was then added followed by potassium carbonate (2.07 g, 14.98 mmol). The mixture was then cooled to 0° C. in a brine ice bath. Benzyl chloroformate (1.06 mL, 7.49 mmol) was then added dropwise, and the mixture was allowed to warm to room temperature and stirred overnight. Afterwards, the reaction was quenched with 20 mL water and extracted with 50 mL ethyl acetate. The organic layer was separated and washed with saturated ammonium chloride solution and brine. The organic layer was then dried over anhydrous sodium sulfate and concentrated in vacuo to afford an orange oil. Flash chromatography (0-50% ethyl acetate in hexanes) afforded tert-butyl(2S)-2-(benzyloxycarbonylaminomethyl)pyrrolidine-1-carboxylate (13a)(2330 mg, 6.9675 mmol, 93% yield) as a colorless oil. The corresponding ¹H NMR spectrum corresponds with the literature (Japanese Patent Application No. JP 2009298710).

### B. Preparation of tert-butyl(S)-2-((((benzyloxy)carbonyl)amino)methyl)piperidine-1-carboxylate (13b)

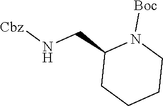 [SMILES: CC(C)(C)OC(=O)N1CCCC[C@H]1CNC(=O)OCc1ccccc1]

An oven-dried 100 mL Schlenk tube was charged with a stir bar and tert-butyl(2S)-2-(aminomethyl)piperidine-1-carboxylate (12b)(1.50 g, 7 mmol) and placed under argon. Anhydrous THF (20 mL) was then added followed by potassium carbonate (1.93 g, 14 mmol). The mixture was then cooled to 0° C. in a brine ice bath. Benzyl chloroformate (1.04 ml, 7.35 mmol) was then added dropwise, and the mixture was allowed to warm to room temperature and stirred overnight. Afterward, the reaction was quenched with water (20 mL) and extracted with ethyl acetate (50 mL). The organic layer was separated and washed with saturated ammonium chloride solution and brine. The organic layer was then dried over anhydrous sodium sulfate and concentrated in vacuo to afford an orange oil. Flash chromatography (0-50% ethyl acetate in hexanes) tert-butyl(S)-2-((((benzyloxy)carbonyl)amino)methyl)piperidine-1-afforded carboxylate (2120 mg, 6.0843 mmol, 87% yield) as a colorless oil. ¹H NMR (600 MHZ, CDCl₃) δ 7.38-7.27 (m, 5H), 5.16-4.98 (m, 1H), 5.08 (s, 2H), 4.34 (brs, 1H), 3.97 (brs, 1H), 3.61 (brs, 1H), 3.22-3.10 (m, 1H), 2.82 (t, J=12.7 Hz, 1H), 1.86-1.48 (m, 5H), 1.42 (s, 10H). ¹³C NMR (151 MHZ, CDCl₃) δ 156.7, 155.8, 136.7, 128.6, 128.1 (2C), 79.9, 66.7, 49.9, 41.1, 39.3, 28.5, 26.5, 25.4, 19.4. HRMS (APCI+) [M+H]⁺ calc. for C₁₉H₂₉N₂O₄, 349.2127, observed, 349.2109.

### C. Preparation of tert-butyl(2S)-2-((benzyloxycarbonyl(methyl)amino)methyl)pyrrolidine-1-carboxylate (14a)

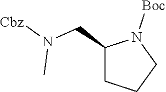 [SMILES: CN(C[C@@H]1CCCN1C(=O)OC(C)(C)C)C(=O)OCc1ccccc1]

An oven-dried 100 mL Schlenk tube was charged with a stirrer bar and tert-butyl(S)-2-((((benzyloxy)carbonyl)amino)methyl)pyrrolidine-1-carboxylate (13a)(2.00 g, 5.98 mmol) under argon. Anhydrous THF (25 mL) was then added, and the mixture was cooled to 0° C. in a brine ice bath. Sodium hydride (251.19 mg, 10.47 mmol) was then added to the mixture. After stirring for 30 minutes, methyl iodide (654.44 μL, 10.47 mmol) was added dropwise to mixture at 0° C., and the resulting mixture was allowed to warm to room temperature and stirred overnight (18 h). Afterward, the reaction mixture was quenched by adding a few drops of DI water and then pouring the mixture into saturated ammonium chloride solution (150 mL). The organic layer was then washed with brine and dried over anhydrous sodium sulfate. Concentration in vacuo afforded an orange oil that was subjected to flash chromatography (0-50% ethyl acetate in hexanes) to afford tert-butyl(2S)-2-((benzyloxycarbonyl(methyl)amino)methyl)pyrrolidine-1-carboxylate (14a) (1.89 g, 5.4242 mmol, 91% yield) as an orange oil. ¹H NMR (600 MHZ, CDCl₃) δ 7.38-7.28 (m, 5H), 5.17-5.05 (m, 2H), 4.05-3.90 (m, 1H), 3.59-3.07 (m, 4H), 2.98 (brs, 3H), 1.95-1.64 (m, 4H), 1.46 (s, 9H). ¹³C NMR (151 MHZ, CDCl₃) δ 157.0, 154.8, 137.1, 128.6, 128.3, 127.9, 79.6, 67.4, 55.4, 50.8, 46.7, 35.4, 28.6, 23.7, 22.7. HRMS (APCI+) [M+H]⁺ calc. for C₁₉H₂₉N₂O₄, 349.2127, observed, 349.2109.

### D. Preparation of tert-butyl(2S)-2-((benzyloxycarbonyl(methyl)amino)methyl)piperidine-1-carboxylate (14b)

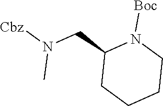 [SMILES: CN(C[C@@H]1CCCCN1C(=O)OC(C)(C)C)C(=O)OCc1ccccc1]

An oven-dried 100 mL Schlenk tube was charged with a stirrer bar and tert-butyl(S)-2-((((benzyloxy)carbonyl)amino)methyl)piperidine-1-carboxylate (13b)(2.00 g, 5.74 mmol) and placed under argon. Anhydrous THF (25 mL) was then added, and the mixture was cooled to 0° C. in a brine ice bath. Sodium hydride (401.79 mg, 10.04 mmol) was then added to the mixture. After stirring for 30 minutes, methyl iodide (628.09 μL, 10.04 mmol) was added dropwise to the mixture at 0° C., and the resulting mixture was allowed to warm to room temperature and stirred overnight (18 h). Afterward, the reaction mixture was quenched by adding a few drops of water and then pouring the mixture into 150 mL saturated ammonium chloride solution. The organic layer was then washed with brine and dried over anhydrous sodium sulfate. Concentration in vacuo afforded an orange oil that was subjected to flash chromatography (0-50% ethyl acetate in hexanes) to afford tert-butyl(2S)-2-((benzyloxycarbonyl(methyl)amino)methyl)piperidine-1-carboxylate (14b)(1.894 g, 5.2254 mmol, 91% yield) as a light-yellow oil. ¹H NMR (600 MHZ, CDCl₃) & 7.37-7.28 (m, 5H), 5.17-5.05 (m, 2H), 4.42 (brs, 1H), 4.11-3.83 (m, 1H), 3.53-3.12 (m, 2H), 2.95 (s, 3H), 2.94* (s, 3H), 1.70-1.47 (m, 5H), 1.44 (s, 9H), 1.42* (s, 9H), 1.39-1.32 (m, 2H). ¹³C NMR (151 MHZ, CDCl₃) δ 156.6, 155.1, 137.0, 128.6, 128.4, 127.9, 79.7, 79.4*, 67.4, 67.2*, 48.1, 47.2, 38.9, 34.5, 28.5, 26.3, 25.5, 25.4*, 19.4. HRMS (APCI+) [M+H]⁺ calc. for C₂₀H₃₁N₂O₄, 363.2284, observed, 363.2264.

### E. Preparation of tert-butyl(S)-2-((methylamino)methyl)pyrrolidine-1-carboxylate (15a)

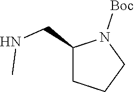

An oven-dried 100 mL round bottom flask was charged with a stirrer bar, tert-butyl(2S)-2-((benzyloxycarbonyl(methyl)amino)methyl)pyrrolidine-1-carboxylate (14a)(500.00 mg, 1.43 mmol), palladium on carbon (3.23 mg, 0.0300 mmol), and anhydrous methanol (5 mL) under argon. A T-joint bearing a hydrogen balloon and Schlenk line connection was affixed to the flask. The reaction flask was then briefly evacuated and filled with hydrogen. This process of hydrogen filling was repeated 3 times. The mixture was then allowed to stir at room temperature under hydrogen for 2 h. Afterward, the mixture was filtered over celite, with the celite further washed with methanol. The combined eluants were then concentrated in vacuo to afford a colorless oil that was immediately carried forward to the next reaction.

### F. Preparation of tert-butyl(S)-2-((methylamino)methyl)piperidine-1-carboxylate (15b)

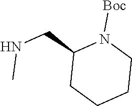 [SMILES: CNC[C@@H]1CCCCN1C(=O)OC(C)(C)C]

An oven-dried 100 mL round bottom flask was charged with a stirrer bar, tert-butyl(2S)-2-((benzyloxycarbonyl(methyl)amino)methyl)piperidine-1-carboxylate (14b)(268.23 mg, 0.7400 mmol), palladium on carbon (3.28 mg, 0.0300 mmol), and anhydrous methanol (5 mL) under argon. A T-joint bearing a hydrogen balloon and Schlenk line connection was affixed to the flask. The reaction flask was then briefly evacuated and filled with hydrogen. This process of hydrogen filling was repeated 3 times. The mixture was then allowed to stir at room temperature under hydrogen for 2 h. Afterward, the mixture was filtered over celite, with the celite further washed with methanol. The combined eluants were then concentrated in vacuo to afford a colorless oil that was immediately carried forward to the next reaction.

### Example 4. Preparation of tert-butyl(S)-methyl(pyrrolidin-2-ylmethyl)carbamate (19)

tert-Butyl(S)-methyl(pyrrolidin-2-ylmethyl)carbamate (19) was prepared according to Scheme 4.

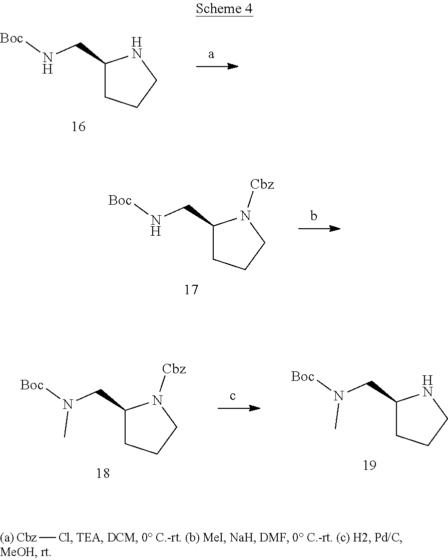 [SMILES: CC(C)(C)OC(=O)NC[C@@H]1CCCN1 ; CC(C)(C)OC(=O)NC[C@@H]1CCCN1C(=O)OCc1ccccc1 ; CN(C[C@@H]1CCCN1C(=O)OCc1ccccc1)C(=O)OC(C)(C)C ; CN(C[C@@H]1CCCN1)C(=O)OC(C)(C)C ; O=C(Cl)OCc1ccccc1]

### A. Preparation of benzyl(S)-2-([(tert-butoxycarbonyl)amino]methyl)pyrrolidine-1-carboxylate (17)

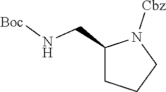 [SMILES: CC(C)(C)OC(=O)NC[C@@H]1CCCN1C(=O)OCc1ccccc1]

To an oven-dried 2-neck round bottom flask, equipped with a stirrer bar, was added tert-butyl(S)-(pyrrolidin-2-ylmethyl)carbamate (16)(302 mg, 200.28 mmol) and anhydrous DCM (3 mL). The solution was then placed in an ice bath, and TEA (0.84 mL, 6.04 mmol) was added, followed by the drop-wise addition of benzyl chloroformate (1.25 g, 7.32 mmol) diluted with anhydrous DCM (1.5 mL). The reaction mixture was stirred at 0° C. and acclimated to room temperature over 3 h. Thereafter, ethyl acetate (15 mL) was added and extracted with DI water and then saturated sodium bicarbonate solution (15 mL). The organic layer was washed with brine, dried over anhydrous magnesium sulfate, filtered, and concentrated in vacuo. Flash chromatography (0-40% ethyl acetate/hexane) afforded the product benzyl(S)-2-([(tert-butoxycarbonyl)amino]methyl)pyrrolidine-1-carboxylate (17)(460 mg, 1.38 mmol, 91%) as a viscous clear oil. ¹H and ¹³C NMR spectra correspond to the literature (Dal Corso, et al., Angew. Chem. Int. Ed., 2020, 59 (10), 4176-4181).

### B. Preparation of benzyl(S)-2-(((tert-butoxycarbonyl)(methyl)amino)methyl)pyrrolidine-1-carboxylate (18)

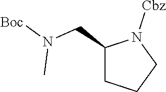 [SMILES: CN(C[C@@H]1CCCN1C(=O)OCc1ccccc1)C(=O)OC(C)(C)C]

An oven-dried 50 mL 2-neck round bottom flask was charged with a stirrer bar and benzyl (S)-2-([(tert-butoxycarbonyl)amino]methyl)pyrrolidine-1-carboxylate (17)(440 mg, 1.316 mmol) under argon. Anhydrous DMF (3 mL) was then added, and the mixture was cooled to 0° C. in an ice bath. Methyl iodide (0.41 mL, 6.58 mmol) was added dropwise to the mixture at 0° C., followed by the addition of sodium hydride (60% in mineral oil)(120 mg, 3.02 mmol) in one portion. The reaction mixture was stirred and allowed to acclimate to room temperature overnight. Afterward, the reaction mixture was quenched by adding a few drops of DI water and diluted with 20 mL ethyl acetate. The solution was then extracted with 15 mL saturated ammonium chloride solution. The organic layer was washed with brine (×2), dried over anhydrous magnesium sulfate, filtered, and concentrated in vacuo. Flash chromatography (0-30% ethyl acetate/hexanes) afforded(S)-2-(((tert-butoxycarbonyl)(methyl)amino)methyl)pyrrolidine-1-carboxylate (18)(388 mg, 1.11 mmol, 85%) as a clear oil. ¹H and ¹³C NMR spectra correspond to the literature (Dal Corso, et al., Angew. Chem. Int. Ed., 2020, 59 (10), 4176-4181).

### C. Preparation of tert-butyl(S)-methyl(pyrrolidin-2-ylmethyl)carbamate (19)

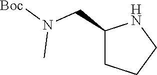

An oven-dried 100 mL round bottom flask was charged with a stirrer bar, benzyl(S)-2-(((tert-butoxycarbonyl)(methyl)amino)methyl)pyrrolidine-1-carboxylate (18)(370 mg, 1.062 mmol), palladium on carbon (28 mg, 0.27 mmol), and 7 mL methanol under an argon atmosphere. A T-joint bearing a hydrogen balloon and Schlenk line connection was affixed to the flask. The reaction flask was then briefly evacuated and filled with hydrogen (×2). The mixture was then allowed to stir at room temperature under hydrogen for 2 h. The mixture was filtered over celite and washed with 10 mL methanol. The combined eluants were then concentrated in vacuo. Flash chromatography (2-15% MeOH/DCM) afforded tert-butyl(S)-methyl(pyrrolidin-2-ylmethyl)carbamate (19)(184 mg, 0.856 mmol, 80%) as a clear oil. ¹H NMR spectra correspond to the literature (Dal Corso, et al., Angew. Chem. Int. Ed., 2020, 59 (10), 4176-4181).

### Example 5. Preparation of (2S,4R)-4-((tert-butyldimethylsilyl)oxy)-2-((methylamino)methyl)pyrrolidine-1-carboxylate (26)

(2S,4R)-4-((tert-Butyldimethylsilyl)oxy)-2-((methylamino)methyl)pyrrolidine-1-carboxylate (26) was prepared according to Scheme 5.

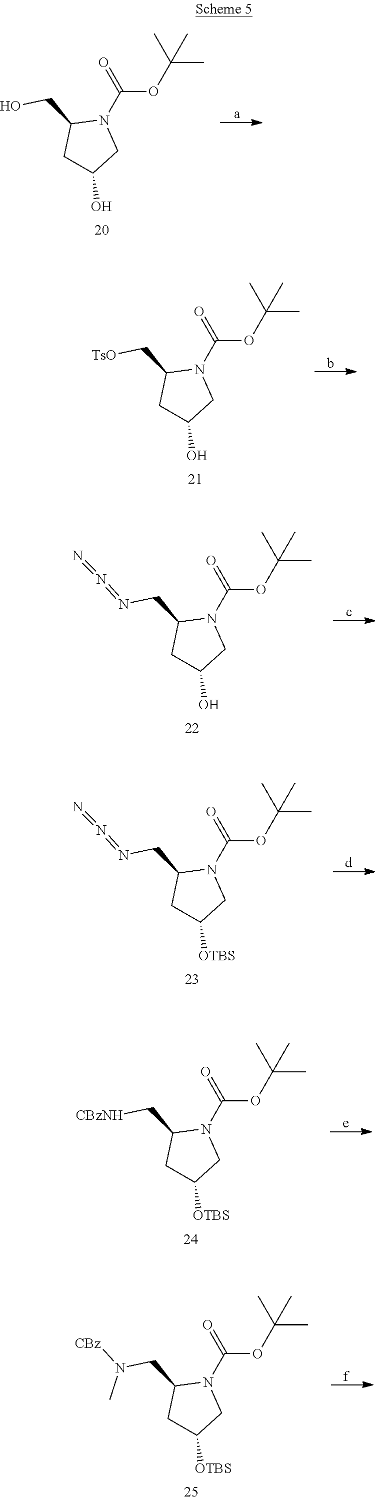

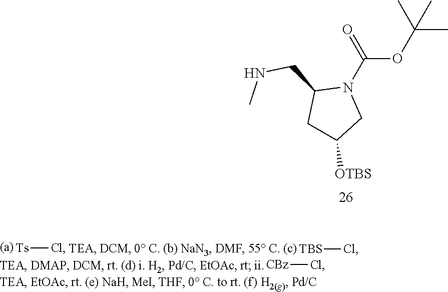

### A. Preparation of ert-butyl(2S,4R)-4-hydroxy-2-((tosyloxy)methyl)pyrrolidine-1-carboxylate (21)

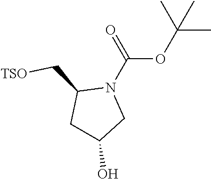

A flame-dried 250 mL 2-neck round bottom flask was charged with a stirrer bar and tert-butyl(2S,4R)-4-hydroxy-2-(hydroxymethyl)pyrrolidine-1-carboxylate (20, purchased commercially)(2.0 g, 9.21 mmol) under argon. Anhydrous DCM (50 mL) and triethylamine (2.98 mL, 21.17 mmol) were then added. The mixture was cooled to 0° C. in a brine ice bath. p-Toluenesulfonyl chloride (2.02 g, 10.59 mmol) was then added as one portion, and the resulting mixture was allowed to warm to room temperature overnight while stirring. After 20 h, the mixture was diluted in 50 mL DCM and washed with brine solution (100 mL). The organic layer was then dried over anhydrous sodium sulfate, filtered, and concentrated in vacuo to afford a light-yellow oil. Flash chromatography (40 g silica, 0-100% ethyl acetate in hexanes) afforded two major products including the desired tert-butyl(2S,4R)-4-hydroxy-2-(p-tolylsulfonyloxymethyl)pyrrolidine-1-carboxylate (21)(1500 mg, 4.0382 mmol, 44% yield) as a colorless oil. ¹H and ¹³C NMR correspond to the literature (Beinat, et al., Tetrahedron Lett., 2013, 54, 5345-5347).

### B. Preparation of tert-butyl(2S,4R)-2-(azidomethyl)-4-hydroxypyrrolidine-1-carboxylate (22)

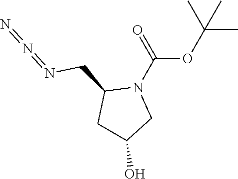

A flame-dried 100 mL round bottom was charged with a stirrer bar and sodium azide (787.58 mg, 12.11 mmol) under argon. tert-Butyl (2S,4R)-4-hydroxy-2-(p-tolylsulfonyloxymethyl)pyrrolidine-1-carboxylate (21)(1500.00 mg, 4.04 mmol) as a solution in anhydrous DMF (10 mL) was then added. The resulting mixture was then heated to 55° C. for 48 h. Afterward, the mixture was diluted in 50 mL ethyl acetate and extracted with brine 3×100 mL. The organic layer was then dried over anhydrous sodium sulfate, filtered, and concentrated to afford a light-yellow oil. Flash chromatography afforded tert-butyl(2S,4R)-2-(azidomethyl)-4-hydroxy-pyrrolidine-1-carboxylate (22)(470 mg, 1.94 mmol, 48% yield) as a colorless oil. ¹H NMR corresponds with the literature (Beinat, et al., Tetrahedron Lett., 2013, 54, 5345-5347).

### C. Preparation of tert-butyl(2S,4R)-2-(azidomethyl)-4-((tert-butyldimethylsilyl)oxy)pyrrolidine-1-carboxylate (23)

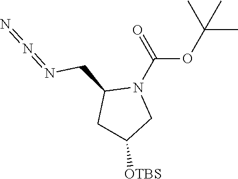

An oven-dried 100 mL round bottom flask was charged with a stirrer bar, tert-butyl (2S,4R)-2-(azidomethyl)-4-hydroxy-pyrrolidine-1-carboxylate (22)(450.00 mg, 1.86 mmol), 4-dimethylaminopyridine (11.35 mg, 0.0900 mmol), anhydrous DCM (5 mL), TEA (0.39 mL, 2.79 mmol), and tert-butyldimethylchlorosilane (419.93 mg, 2.79 mmol) under argon. The mixture was allowed to stir overnight at room temperature. Afterward, the mixture was diluted in 50 mL DCM and washed with brine. The organic layer was then dried over anhydrous sodium sulfate, filtered, and concentrated to afford a colorless oil. Flash chromatography (0-20% ethyl acetate in hexanes) afforded tert-butyl(2S,4R)-2-(azidomethyl)-4-(tert-butyl(dimethyl) silyl)oxy-pyrrolidine-1-carboxylate (23)(510 mg, 1.4304 mmol, 77% yield) as a white solid. ¹H and ¹³C NMR spectra correspond to the literature (Zheng, et al., Chem. Comm., 2013, 49 (40), 4561-4563).

### D. Preparation of tert-butyl(2S,4R)-2-((((benzyloxy)carbonyl)amino)methyl)-4-((tert-butyldimethylsilyl)oxy)pyrrolidine-1-carboxylate (24)

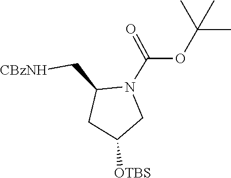

An oven-dried 2-neck 100 mL round bottom flask was affixed with a 3-way adapter controlled via PTFE stopcock with a hydrogen balloon and house vacuum attached. Palladium on carbon (73.72 mmol), mg, 0.0700 tert-butyl(2S,4R)-2-(azidomethyl)-4-(tert-butyl(dimethyl) silyl)oxy-pyrrolidine-1-carboxylate (23)(494.00 mg, 1.39 mmol), and anhydrous ethyl acetate (10 mL) under argon. The flask was then placed under hydrogen gas via three rounds of purge-refill using the 3-way adapter. The resulting black heterogenous mixture was allowed to stir at room temperature for 3 h. Afterward, the mixture was filtered over a pad of celite into a flame-dried 250 mL round bottom flask charged with a stirrer bar. The flask containing the filtrate was then purged with argon and cooled to 0° C. in a brine ice bath. Anhydrous TEA (0.19 mL, 1.52 mmol) was then added followed by the dropwise addition of benzyl chloroformate (0.22 mL, 1.52 mmol). The resulting mixture was allowed to warm to room temperature and stirred overnight. Afterward, the mixture was diluted in 50 mL ethyl acetate, washed with 1M HCl_{(aq)} (5 mL), 10% sodium bicarbonate solution (50 mL), and brine (100 mL). The organic layer was then dried over anhydrous sodium sulfate, filtered, and concentrated in vacuo to afford the crude. Flash chromatography (0-40% ethyl acetate in hexanes) afforded tert-butyl(2S,4R)-2-((((benzyloxy)carbonyl)amino)methyl)-4-((tert-butyldimethylsilyl)oxy)pyrrolidine-1-carboxylate (24)(550 mg, 1.1836 mmol, 85% yield) as a colorless oil. ¹H NMR (600 MHZ, CDCl₃) δ 7.37-7.29 (m, 5H), 6.02 (s, 1H), 5.14-5.03 (m, 2H), 4.30 (t, J=4.1 Hz, 1H), 4.07-3.98 (m, 1H), 3.53-3.19 (m, 4H), 2.00-1.93 (m, 1H), 1.76 (s, 1H), 1.45 (s, 9H), 0.86 (s, 9H), 0.04 (s, 6H). ¹³C NMR (151 MHZ, CDCl₃) δ 157.0, 156.5, 137.0, 128.6, 128.2, 128.1, 80.1, 70.1, 66.6, 56.3, 55.7, 45.9, 44.6, 38.9, 28.5, 25.8, 18.1, −4.8. HRMS (APCI+) [M+H]⁺ calc. for C₂₄H₄₁N₂O₅Si, 465.277921, observed, 465.275900.

### E. Preparation of tert-butyl(2S,4R)-2-((((benzyloxy)carbonyl)(methyl)amino)methyl)-4-((tert-butyldimethylsilyl)oxy)pyrrolidine-1-carboxylate (25)

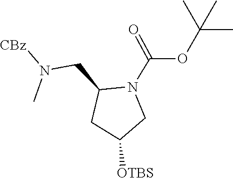

A 50 mL Schlenk tube equipped with a stir bar was charged with tert-butyl(2S,4R)-2-((((benzyloxy)carbonyl)amino)methyl)-4-((tert-butyldimethylsilyl)oxy)pyrrolidine-1-carboxylate (24)(500.00 mg, 1.08 mmol) and placed under argon. Anhydrous THF (10 mL) was then added, and the resulting mixture was cooled to 0° C. in a brine ice bath. Sodium hydride (71.00 mg, 1.78 mmol) was then added in one portion followed by dropwise addition of methyl iodide (0.11 mL, 1.76 mmol). The mixture was allowed to warm to room temperature and stir overnight. Afterward, the mixture was quenched with saturated ammonium chloride solution. The aqueous layer was then extracted with ethyl acetate (20 mL). The combined organic layers were then dried over anhydrous sodium sulfate, filtered, and concentrated in vacuo to afford the crude as a colorless oil. Flash chromatography (0-30% ethyl acetate in hexanes) afforded tert-butyl(2S,4R)-2-((((benzyloxy)carbonyl)(methyl)amino)methyl)-4-((tert-butyldimethylsilyl)oxy)pyrrolidine-1-carboxylate (25)(424 mg, 0.8857 mmol, 82% yield) as a colorless oil. ¹H NMR (600 MHZ, CDCl₃) δ 7.38-7.28 (m, 5H), 5.22-5.03 (m, 2H), 4.41-4.22 (m, 1H), 4.15-4.00 (m, 1H), 3.57-3.26 (m, 4H), 2.99-2.90 (m, 3H), 2.01-1.76 (m, 2H), 1.45 (s, 9H), 0.85 (s, 9H), 0.84* (s, 9H) 0.02 (s, 6H), 0.00* (s, 6H). ¹³C NMR (151 MHZ, CDCl₃) δ 156.4, 155.1, 136.9, 128.5, 127.9, 127.7, 79.7, 70.0, 67.1, 54.7, 54.3, 50.9, 37.8, 35.0, 28.5, 25.7, 18.0, −4.9. HRMS (APCI+) [M+H]⁺ calc. for C₂₅H₄₃N₂O₅Si, 479.2936, observed, 479.2917.

### F. Preparation of tert-butyl(2S,4R)-4-((tert-butyldimethylsilyl)oxy)-2-((methylamino)methyl)pyrrolidine-1-carboxylate (26)

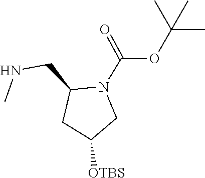 [SMILES: CNC[C@@H]1C[C@@H](O[Si](C)(C)C(C)(C)C)CN1C(=O)OC(C)(C)C]

A 100 mL round bottom equipped with a stirrer bar was charged with tert-butyl(2S,4R)-2-((((benzyloxy)carbonyl)(methyl)amino)methyl)-4-((tert-butyldimethylsilyl)oxy)pyrrolidine-1-carboxylate (25)(410.00 mg, 0.8600 mmol) and anhydrous methanol (5 mL). The flask was then purged with argon for 15 minutes and charged with palladium on carbon (9.1 mg, 0.09 mmol). A 3-way T-joint with a hydrogen balloon affixed was then attached to the flask. The flask was then briefly evacuated followed by filling with hydrogen. This process of vacuum-hydrogen filling was repeated three times. The reaction was then allowed to stir at room temperature for 1 h. Afterward, the mixture was filtered over a pad of celite and concentrated in vacuo to afford the title compound which was immediately carried forward to the next reaction.

### Example 6. Preparation of Prodrugs of Progesterone C20-Oxime

Prodrugs of progesterone C20-oxime were prepared according to the general procedures described below. A representative synthesis of 32b is shown below in Scheme 6.

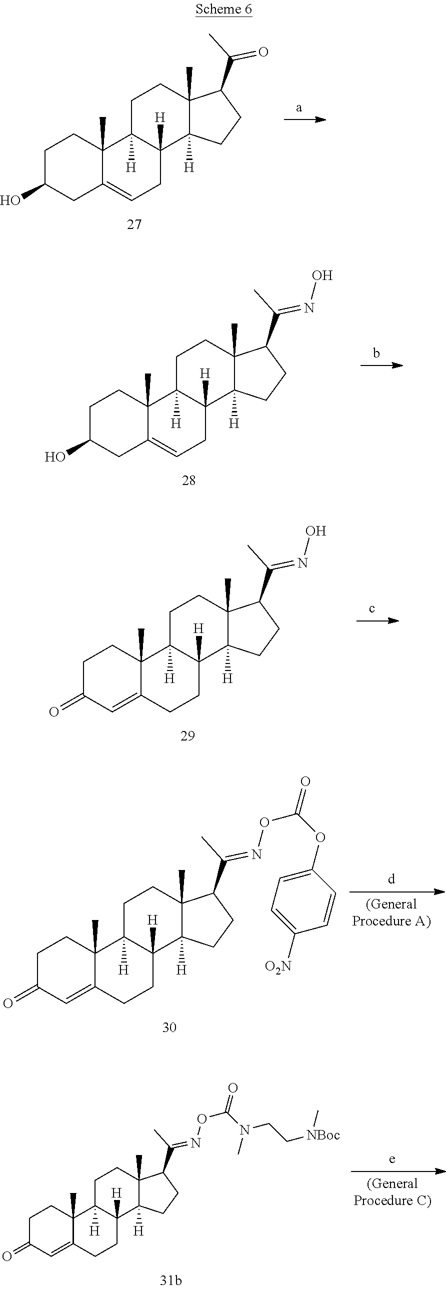

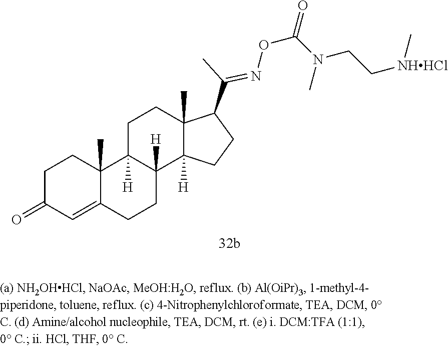

General procedure A: Compound 30 (1 equiv.), DCM (5 mL), triethylamine (or diisopropylethylamine)(2 equiv.), and amine nucleophile (1.1 equiv.) were combined at room temperature. The mixture was allowed to stir until consumption of the starting material was observed by TLC (30-60 min). The mixture was then concentrated in vacuo to afford a yellow oil. Flash chromatography (ethyl acetate:DCM) afforded the product as a white solid.

General procedure B: A flame-dried 100 mL round bottom flask was charged with a stirrer bar, compound 30 (1 equiv.), anhydrous DCM (10 mL), amine (1 equiv.), and 4-dimethylaminopyridine (1.5 equiv.) under argon. The resulting mixture was allowed to stir for 20 h. Afterward, the solution was diluted in DCM (50 mL) and washed with brine (3×100 mL brine). The organic layer was then dried over anhydrous sodium sulfate and concentrated in vacuo to afford a yellow oil. Flash chromatography (ethyl acetate:DCM) afforded the product as a white solid.

General procedure C: To a flame-dried vial containing a stirred bar and Boc-protected amine (1 equiv.) was added anhydrous DCM (1 mL) under argon. The resulting solution was then cooled to 0° C. in a brine ice bath. Trifluoroacetic acid (TFA, 1 mL) was then added dropwise, and the resulting solution was stirred at 0° C. for 1 h. The resulting solution was then concentrated in vacuo to afford a pink oil. The resulting oil was then dissolved in THF (0.6000 mL) and cooled to 0° C. Hydrochloric acid (1.5 equiv.)(4 M in dioxane) was then added dropwise. After stirring for 30 min at 0° C., diethyl ether (10 mL) was then added dropwise to the solution at 0° C. and resulted in formation of a white precipitate. The mixture was then filtered over a filter frit and washed with diethyl ether (3× 10 mL) to afford the hydrochloride salt as a white solid.

### AA. Preparation of (E)-1-((3S,8S,9S,10R,13S,14S,17S)-3-hydroxy-10,13-dimethyl-2,3,4,7,8,9,10,11,12,13,14,15,16,17-tetradecahydro-1H-cyclopenta[a]phenanthren-17-yl)ethan-1-one oxime (28)

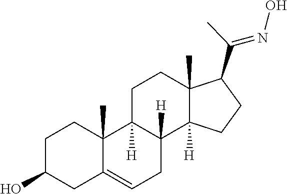

To a 3-neck 500 mL RB flask with a stirrer bar, condenser, and 50 mL addition funnel was added 3β-hydroxy-5-pregnen-20-one (27)(10.00 g, 31.6 mmol) and methanol (250 mL). The mixture was heated to reflux to dissolve the suspended solid (ca. 10 min). A separate solution containing sodium acetate (5.70 g, 69.51 mmol), hydroxylamine hydrochloride (4.39 g, 63.2 mmol), and water (15 mL) was then slowly added dropwise via addition funnel (ca. 30 min). After addition, the mixture was allowed to reflux until complete conversion as observed by TLC (3 h). A thick white precipitate formed during the course of the reaction. After completion, the mixture was allowed to cool to room temperature and an additional 150 mL of water was added, followed by 30 min of stirring. The reaction mixture was then subjected to vacuum filtration, and the resulting white solid was washed with water (3×100 mL). Drying of the solid in vacuo afforded the product (E)-1-((3S,8S,9S,10R,13S,14S,17S)-3-hydroxy-10,13-dimethyl-2,3,4,7,8,9,10,11,12,13,14,15,16,17-tetradecahydro-1H-cyclopenta[a]phenanthren-17-yl)ethan-1-one oxime (28)(9.3 g, 28.055 mmol, 89% yield) as a white solid. ¹H NMR corresponds with the literature (Guthrie, et al., ACS Med. Chem. Lett., 2012, 3 (5), 362-366).

### AB. Preparation of (8S,9S,10R,13S,14S,17S)-17-[(E)-N-hydroxy-C-methyl-carbonimidoyl]-10,13-dimethyl-1,2,6,7,8,9,11,12,14,15,16,17-dodecahydrocyclopenta[a]phenanthren-3-one (29)

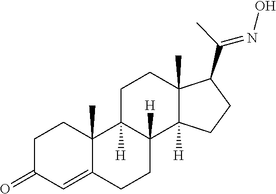

To an oven-dried 3-neck 100 mL round bottom flask with a stir bar, 4 Å molecular sieves, and condenser affixed was added (E)-1-((3S,8S,9S,10R,13S, 14S,17S)-3-hydroxy-10,13-dimethyl-2,3,4,7,8,9,10,11,12, 13, 14, 15, 16, 17-tetradecahydro-1H-cyclopenta[a]phenanthren-17-yl)ethan-1-one oxime (28)(5.00 g, 15.08 mmol) and subsequently placed under an argon atmosphere via Schlenk technique. Toluene (125 mL) and 1-methyl-4-piperidone (27.83 mL, 226.25 mmol) were then added and the mixture was heated to reflux. Aluminum isopropoxide (6.16 g, 30.17 mmol) was then added portion-wise. The resulting mixture was allowed to reflux for 8.5 h. The mixture was then allowed to cool to room temperature and saturated Rochelle's salt solution (30 mL) was added to the mixture and allowed to stir for an additional 20 min. The mixture was then extracted with DCM (3×50 mL). The organic layer was then washed with brine (3×50 mL) and dried over anhydrous sodium sulfate. Concentration in vacuo afforded an orange oil that was subjected to silica plug, following elution with 250 mL of 1:1 DCM:ethyl acetate. Concentration of the eluant followed by flash chromatography (5-20% ethyl acetate in DCM) followed by a second column (5-80% ethyl acetate in DCM) afforded the product (8S,9S,10R,13S, 14S, 17S)-17-[(E)-N-hydroxy-C-methyl-carbonimidoyl]-10,13-dimethyl-1,2,6,7,8,9,11, 12, 14, 15, 16, 17-dodecahydrocyclopenta[a]phenanthren-3-one (29)(2210 mg, 6.7075 mmol, 44% yield) as a white solid. ¹H NMR corresponds with the literature (Guthrie, et al., ACS Med. Chem. Lett., 2012, 3 (5), 362-366).

### AC. Preparation of (8S,9S,10R,13S,14S,17S)-10,13-dimethyl-17-((E)-1-((((4-nitrophenoxy)carbonyl)oxy)imino)ethyl)-1,2,6,7,8,9,10,11,12,13,14,15,16,17-tetradecahydro-3H-cyclopenta[a]phenanthren-3-one (30)

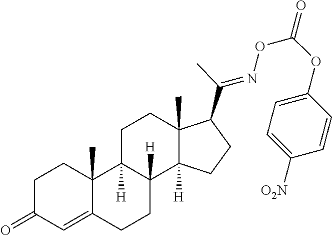

To a flame-dried 100 mL round bottom flask with a stirrer bar was added (8S,9S,10R,13S, 14S, 17S)-17-[(E)-N-hydroxy-C-methyl-carbonimidoyl]-10,13-dimethyl-1,2,6,7,8,9,11, 12, 14, 15, 16, 17-dodecahydrocyclopenta[a]phenanthren-3-one (29)(500.00 mg, 1.52 mmol), TEA (317.27 μL, 2.28 mmol) and anhydrous DCM (30 mL) under argon. The mixture was then cooled to 0° C. in a brine ice bath. 4-Nitrophenyl chloroformate (367 mg, 1.82 mmol) was then added portion-wise and the resulting mixture was allowed to stir at 0° C. for 1.5 h. Afterward, the mixture was concentrated in vacuo, adsorbed onto celite, and subjected to flash chromatography (40g silica, 0-20% over 15 min) to afford the title compound as a white solid (701 mg, 1.417 mmol, 93% yield). ¹H NMR (600 MHZ, CDCl₃) δ 8.3 (d, J=9.2 Hz, 2H), 7.4 (d, J=9.2 Hz, 2H), 5.7 (s, 1H), 2.5-2.2 (m, 6H), 2.1 (s, 3H), 2.1-2.0 (m, 1H), 2.0 (dt, J=12.3, 3.4 Hz, 1H), 1.9-1.5 (m, 6H), 1.5 (qd, J=13.1, 3.8 Hz, 1H), 1.4-1.2 (m, 2H), 1.2-1.2 (m, 4H), 1.1-1.0 (m, 1H), 1.0-0.9 (m, 1H), 0.8 (s, 3H). ¹³C NMR (151 MHZ, CDCl₃) δ 199.6, 171.1, 168.0, 155.6, 151.3, 145.6, 125.5, 124.1, 121.9, 56.8, 55.6, 53.8, 44.3, 38.7, 38.6, 35.9, 35.8, 34.1, 32.9, 31.9, 24.2, 23.2, 21.1, 17.5, 17.2, 13.6. HRMS (ESI+) [M+H]⁺ calc. for C₂₈H₃₅O₆N₂, 495.24896, observed, 495.24887.

### AD. Preparation of tert-butyl(2-((((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)amino)ethyl)carbamate (31a)

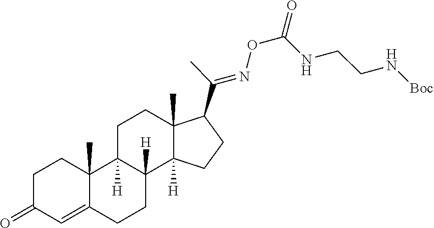

Prepared according to General Procedure A using N-Boc-ethylenediamine as the amine nucleophile with the following exceptions: DMF solvent was used in lieu of DCM. The reaction mixture was diluted in ethyl acetate and washed with saturated lithium bromide solution in addition to brine. The product was in the form of a white solid (280 mg, 0.5430 mmol, 86% yield). ¹H NMR (400 MHZ, CDCl₃) δ 6.7 (t, J=5.7 Hz, 1H), 5.7 (s, 1H), 4.9 (t, J=5.9 Hz, 1H), 3.5-3.2 (m, 4H), 2.4-2.1 (m, 6H), 2.0-2.0 (m, 4H), 1.9-1.8 (m, 3H), 1.8-1.7 (m, 3H), 1.7-1.5 (m, 2H), 1.4 (s, 9H), 1.4-1.2 (m, 3H), 1.2 (s, 3H), 1.1-0.9 (m, 2H), 0.7 (s, 3H). ¹³C NMR (151 MHZ, CDCl₃) δ 199.4, 170.9, 163.8, 156.4 (2C), 124.0, 79.5, 56.8, 55.5, 53.7, 44.0, 41.6, 40.5, 38.6, 38.6, 35.8, 35.7, 33.9, 32.8, 31.8, 28.4, 24.0, 23.0, 21.0, 17.4, 17.3, 13.4. HRMS (ESI+) [M+H]⁺ calc. for C₂₉H₄₆O₅N₃, 516.34320, observed, 516.34315.

### AE. Preparation of tert-butyl(2-((((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)(methyl)amino)ethyl)(methyl)carbamate)(31b)

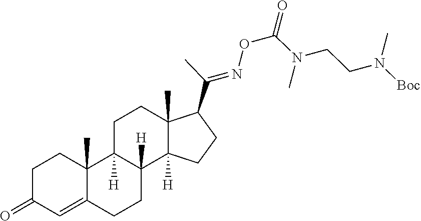

Prepared according to General Procedure A using tert-butyl N-methyl-N-[2-(methylamino)ethyl]carbamate with the following exceptions: DMF solvent was used in lieu of DCM. The reaction mixture was diluted in ethyl acetate and washed with saturated lithium bromide solution in addition to brine. The product was in the form of a white solid (236 mg, 0.4340 mmol, 69% yield). ¹H NMR (600 MHZ, CDCl₃) δ 5.7 (s, 1H), 3.4-3.3 (m, 4H), 3.0 (s, 3H), 2.9 (s, 3H), 2.5-2.2 (m, 6H), 2.0 (ddd, J=13.4, 5.0, 3.1 Hz, 1H), 2.0-1.9 (m, 4H), 1.9-1.8 (m, 1H), 1.8-1.7 (m, 3H), 1.6-1.5 (m, 2H), 1.5-1.4 (m, 10H), 1.4-1.2 (m, 2H), 1.2 (s, 3H), 1.2-1.1 (m, 1H), 1.1-1.0 (m, 1H), 1.0-0.9 (m, 1H), 0.7 (s, 3H). ¹³C NMR (151 MHZ, CDCl₃) δ 199.6, 171.2, 164.7, 155.7, 154.8, 124.0, 79.7, 56.9, 55.5, 53.9, 47.9, 46.9, 44.2, 38.7, 38.6, 35.9, 35.8, 35.3, 34.8, 34.1, 32.9, 32.0, 28.5, 24.3, 23.2, 21.1, 17.5, 17.0, 13.6. HRMS (ESI+) [M+H]⁺ calc. for C₃₂H₄₆ON₇, 544.37584, observed, 544.37444.

### AF. Preparation of tert-butyl(2-((2-((tert-butyldimethylsilyl)oxy)ethyl)(((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)amino)ethyl)(methyl)carbamate (31c)

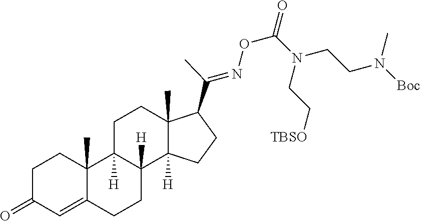

Prepared according to General Procedure A using amine 6 as the amine nucleophile. Rf=0.3 (20% ethyl acetate/DCM. The product was in the form of a white solid (531 mg, 0.772 mmol, 93% yield). ¹H NMR (600 MHZ, CDCl₃) δ 5.73 (s, 1H), 3.83-3.70 (m, 1H), 3.57-3.32 (m, 6H), 2.89 (br s, 3H), 2.46-2.24 (m, 6H), 2.06-1.99 (m, 1H), 1.97-1.89, (m, 4H), 1.86-1.83 (m, 1H), 1.80-1.66 (m, 3H), 1.63-1.50 (m, 2H), 1.45 (br s, 9H), 1.42-1.39 (m, 1H), 1.36-1.21 (m, 3H), 1.18 (s, 3H), 1.17-1.10 (m, 1H), 1.05 (qd, J=13.0, 3.8 Hz, 1H), 1.00-0.94 (m, 2H), 0.87 (s, 9H), 0.72 (s, 3H), 0.03 (s, 6H). ¹³C NMR (151 MHZ, CDCl₃) δ 199.6, 171.3, 164.6, 155.6, 154.9, 124.0, 79.8, 61.7, 56.87, 55.52, 53.88, 50.3, 47.8, 46.8, 45.5, 44.1, 38.7, 38.6, 35.9, 35.8, 34.1, 32.9, 32.0, 28.5, 26.0, 24.3, 23.2, 21.1, 18.4, 17.4, 17.0, 13.6, −5.3. HRMS (APCI+) [M+H]⁺ calc. for C₃₈H₆₆N₃O₆Si, 688.47154, observed, 688.47159.

### AG. Preparation of tert-butyl(2-((tert-butyldimethylsilyl)oxy)ethyl)(2-((((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)(methyl)amino)ethyl)carbamate (31d)

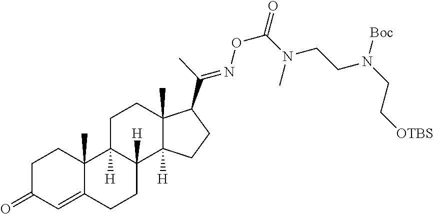

Prepared according to General Procedure A using amine nucleophile 11a. Rf=0.45 (20% ethyl acetate/DCM). The product was in the form of a white solid (507 mg, 0.737 mmol, 89% yield). ¹H NMR (600 MHZ, CDCl₃) δ 5.72 (s, 1H), 3.75-3.62 (m, 1H), 3.51-3.22 (m, 6H), 2.97 (br s, 3H), 2.45-2.24 (m, 6H), 2.05-1.98 (m, 1H), 1.96-1.90, (m, 4H), 1.88-1.75 (m, 2H), 1.74-1.65 (m, 2H), 1.62-1.50 (m, 2H), 1.45 (s, 9H), 1.45* (s, 9H), 1.42-1.39 (m, 2H), 1.37-1.22 (m, 3H), 1.18 (s, 3H), 1.17-1.11 (m, 1H), 1.05 (qd, J=13.0, 3.8 Hz, 1H), 1.00-0.94 (m, 2H), 0.87 (s, 9H), 0.72 (s, 3H), 0.03 (s, 6H). ¹³C NMR (151 MHZ, CDCl₃) δ 199.6, 171.3, 164.6, 155.6, 154.9, 124.0, 79.8, 61.7, 56.87, 55.52, 53.88, 50.3, 47.8, 46.8, 45.5, 44.1, 38.7, 38.6, 35.9, 35.8, 34.1, 32.9, 32.0, 28.5, 26.0, 24.3, 23.2, 21.1, 18.4, 17.4, 17.0, 13.6, −5.3. HRMS (APCI+) [M+H]⁺ calc. for C₃₈H₆₆N₃O₆Si, 688.47154, observed, 687.47132.

### AH. Preparation of tert-butyl(3-((((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)amino) propyl)carbamate (31e)

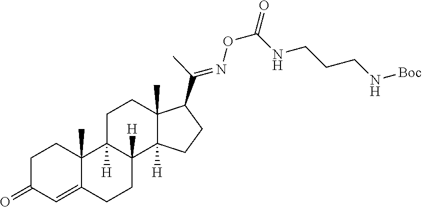

Prepared according to General Procedure A using N-Boc-1,3-propanediamine as the amine nucleophile with the following exceptions: DMF solvent was used in lieu of DCM. The reaction mixture was diluted in ethyl acetate and washed with saturated lithium bromide solution in addition to brine. The product was in the form of a white solid (245 mg, 0.4625 mmol, 73% yield). ¹H NMR (400 MHZ, CDCl₃) δ 6.8 (s, 1H), 5.7 (s, 1H), 4.8 (s, 1H), 3.3 (q, J=6.4 Hz, 2H), 3.2 (q, J=6.3 Hz, 2H), 2.5-2.2 (m, 6H), 2.1-2.0 (m, 1H), 2.0 (s, 3H), 2.0-1.8 (m, 2H), 1.8-1.5 (m, 8H), 1.4 (s, 9H), 1.4-1.2 (m, 2H), 1.2 (s, 3H), 1.2-0.9 (m, 3H), 0.7 (s, 3H). ¹³C NMR (101 MHz, CDCl₃) δ 199.5, 170.9, 163.4, 156.5, 156.4, 124.0, 79.2, 56.9, 55.5, 53.8, 44.0, 38.6, 38.6, 37.5, 37.0, 35.8, 35.7, 34.0, 32.8, 31.9, 30.5, 28.4, 24.1, 23.0, 21.0, 17.4, 17.3, 13.4. HRMS (ESI+) [M+H]⁺ calc. for C₃₀H₄₈O₅N₃; 530.35885, observed, 530.35979.

### AI. Preparation of tert-butyl(2-((((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)oxy)ethyl)carbamate (31f)

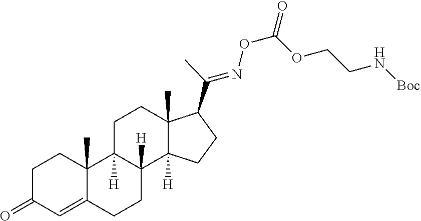

Prepared according to General Procedure B using N-Boc-ethanolamine as the alcohol. The product was in the form of a white solid (241 mg, 0.4664 mmol, 90% yield). ¹H NMR (400 MHZ, CDCl₃) δ 5.7 (s, 1H), 4.9 (s, 1H), 4.3 (t, J=5.2 Hz, 2H), 3.4 (q, J=5.5 Hz, 2H), 2.5-2.2 (m, 6H), 1.9 (s, 5H), 1.9-1.6 (m, 6H), 1.4 (s, 9H), 1.4-1.2 (m, 3H), 1.2 (s, 3H), 1.1-0.9 (m, 3H), 0.7 (s, 3H). ¹³C NMR (101 MHZ, CDCl₃) δ 199.7, 171.2, 166.6, 155.8, 154.0, 126.2, 124.0, 115.8, 67.6, 56.7, 55.5, 53.8, 44.2, 39.8, 38.7, 38.6, 35.8, 34.0, 32.9, 31.9, 28.5, 24.2, 23.1, 21.1, 17.5, 17.0, 13.5. HRMS (APCI+) [M+H]⁺ calc. for C₂₉H₄₅O₆N₂, 517.32721, observed, 517.32751.

### AJ. Preparation of tert-butyl(2S)-2-(((((((E)-1-((8S,9S,10R,13S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)amino)methyl)pyrrolidine-1-carboxylate (31g)

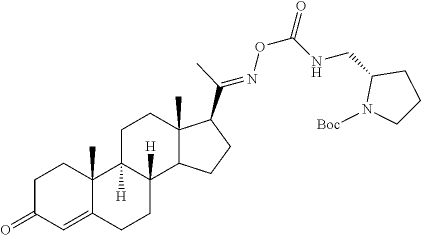

Synthesized according to General Procedure A using amine 12a as the amine nucleophile. The product was in the form of a white solid (297 mg, 0.5344 mmol, 85% yield). ¹H NMR (600 MHz, CDCl₃) δ 7.40 (brs, 1H), 5.73 (s, 1H), 3.95 (brs, 1H), 3.54-3.26 (m, 4H), 2.45-2.24 (m, 6H), 2.04-1.96 (m, 5H), 1.93-1.83 (m, 3H), 1.83-1.66 (m, 5H), 1.63-1.51 (m, 2H), 1.45 (s, 10H), 1.36-1.22 (m, 2H), 1.18 (s, 3H), 1.17-1.13 (m, 1H), 1.09-1.01 (m, 1H), 1.00-0.93 (m, 1H), 0.68 (s, 3H). ¹³C NMR (151 MHZ, CDCl₃) δ 199.6, 171.1, 163.5, 156.5, 155.8, 124.1, 79.7, 57.1, 57.0, 55.6, 53.9, 47.2, 46.1, 44.1, 38.7, 35.9, 35.8, 34.1, 32.9, 32.0, 29.6, 28.6, 24.2, 23.9, 23.1, 21.2, 17.5, 17.4, 13.5. HRMS (ESI+) [M+H]⁺ calc. for C₃₂H₅₀O₅N₃, 556.3745, observed, 556.37416.

### AK. Preparation of tert-butyl(((2S)-1-(((((E)-1-((8S,9S,10R,13S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)pyrrolidin-2-yl)methyl)carbamate (31h)

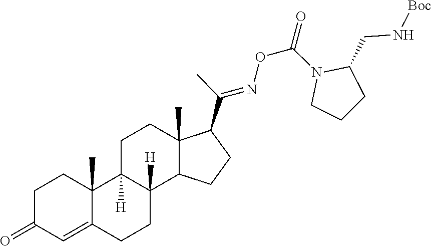

Prepared according to General Procedure A using amine 16 as the amine nucleophile. Rf=0.3 (20% ethyl acetate/DCM. Purified via flash chromatography (10-40% ethyl acetate/DCM). The product was in the form of a white solid (556 mg, 1.000 mmol, quant. yield). ¹H NMR (600 MHz, CDCl₃) δ 5.72 (s, 1H), 4.03-3.93 (m, 1H), 3.59-3.14 (m, 4H), 2.45-2.23 (m, 7H), 2.06-1.95 (m, 1H), 1.94 (s, 3H), 1.93-1.81 (m, 5H), 1.80-1.66 (m, 4H), 1.62-1.50 (m, 2H), 1.41 (br s, 9H), 1.38-1.22 (m, 2H), 1.17 (s, 3H), 1.16-0.93 (m, 4H), 0.72 (s, 3H). ¹³C NMR (151 MHz, CDCl₃) δ 199.6, 171.3, 164.9, 156.6, 154.7, 124.0, 79.1, 58.8, 56.8, 56.1, 55.5, 53.8, 46.8, 44.8, 44.2, 38.7, 38.6, 35.8, 34.1, 32.9, 32.0, 29.3, 28.5, 24.3, 24.2, 23.2, 21.1, 17.5, 17.0, 13.7. HRMS (APCI+) [M+H]⁺ calc. for C₃₂H₅₀N₃O₅, 556.3745, observed, 556.37514.

### AL. Preparation of tert-butyl(2S)-2-(((((((E)-1-((8S,9S,10R,13S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)amino)methyl)piperidine-1-carboxylate (31i)

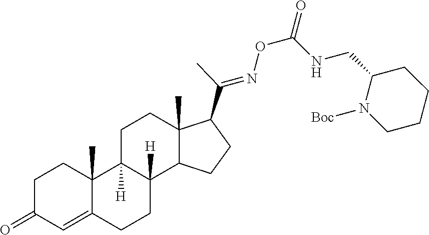

Synthesized according to general procedure A using amine 12b as the amine nucleophile. 302 mg, 0.53 mmol, 87% yield, white solid. TLC (1:4 ethyl acetate/DCM): Rf=0.35. ¹H NMR (600 MHz, CDCl₃) δ 6.45 (s, 1H), 5.72 (s, 1H), 4.40 (s, 1H), 3.96 (s, 1H), 3.62 (s, 1H), 2.87 (s, 1H), 2.47-2.24 (m, 5H), 2.22-2.12 (m, 1H), 2.02-1.99 (m, 1H), 1.97 (s, 3H), 1.97* (s, 3H), 1.93-1.83 (m, 2H), 1.73 (q, J=2.0 Hz, 4H), 1.65-1.49 (m, 7H), 1.44-1.37 (m, 2H), 1.42 (s, 9H), 1.40* (s, 9H), 1.36-1.24 (m, 2H), 1.18 (s, 3H), 1.18-1.11 (m, 1H), 1.10-1.00 (m, 1H), 0.96 (ddd, J=12.2, 10.7, 4.1 Hz, 1H), 0.66 (s, 3H). ¹³C NMR (151 MHZ, CDCl₃) δ 199.4, 170.9, 163.6, 156.0, 155.6, 124.0, 79.5, 56.8, 56.7, 55.4, 55.4, 53.7, 49.7, 44.0, 40.8, 38.6, 38.5, 35.8, 35.7, 33.9, 32.8, 31.8, 28.4, 26.4, 25.3, 24.1, 23.1, 21.0, 19.3, 17.4, 17.0, 13.4. HRMS (ESI+) [M+H]⁺ calc. for C₃₄H₄₈ON₇, 570.39149, observed, 570.39047.

### AM. Preparation of tert-butyl(2S)-2-(((((((E)-1-((8S,9S,10R,13S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)(methyl)amino)methyl)pyrrolidine-1-carboxylate (31j)

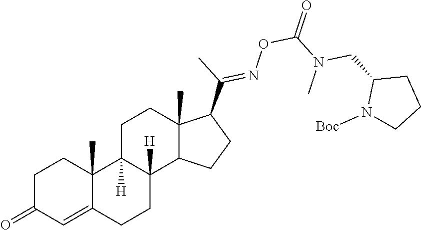

Prepared according to General Procedure A using amine 15a as the amine nucleophile. The product was in the form of a white solid (298 mg, 0.523 mmol, 86% yield). ¹H NMR (600 MHZ, CDCl₃) δ 5.72 (s, 1H), 4.00 (s, 1H), 3.60 (t, J=11.5 Hz, 1H), 3.45-3.10 (m, 3H), 3.03-2.94 (m, 3H), 2.45-2.23 (m, 6H), 2.04-1.90 (m, 2H), 1.93 (s, 3H), 1.76 (s, 8H), 1.62-1.50 (m, 2H), 1.47 (s, 9H), 1.44* (s, 9H), 1.40 (dd, J=13.1, 3.8 Hz, 1H), 1.36-1.22 (m, 2H), 1.17 (s, 3H), 1.16-1.11 (m, 1H), 1.08-0.93 (m, 2H), 0.72 (s, 3H). ¹³C NMR (151 MHZ, CDCl₃) δ 199.6, 171.2, 164.6, 155.8, 154.5, 124.0, 79.3, 56.9, 55.5, 53.9, 51.4, 50.5, 46.6, 46.3, 44.1, 38.7, 38.6, 35.9, 35.8, 34.8, 34.1, 32.9, 32.0, 28.6, 28.1, 24.3, 23.2, 21.1, 17.5, 17.1, 13.6. HRMS (APCI+) [M+H]⁺ calc. for C₃₃H₅₂N₃O₅, 570.3901, observed, 570.3884.

### AN. Preparation of tert-butyl(((2S)-1-(((((E)-1-((8S,9S,10R,13S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)pyrrolidin-2-yl)methyl)(methyl)carbamate (31k)

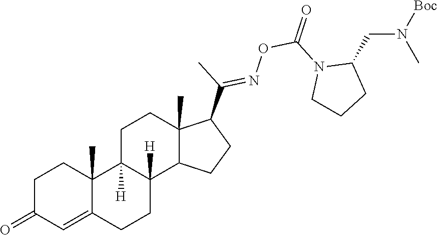

Prepared according to General Procedure A using amine 19 as the amine nucleophile. Purified via flash chromatography (5-30% ethyl acetate/DCM). The product was in the form of a white solid (300 mg, 0.527 mmol, 87% yield). ¹H NMR (600 MHZ, CDCl₃) δ 5.72 (s, 1H), 4.08 (br s, 1H), 3.54-3.11 (m, 4H), 2.90 (s, 3H), 2.45-2.22 (m, 6H), 2.05-1.99 (m, 1H), 1.92 (br s, 3H), 1.88-1.65 (m, 6H), 1.62-1.50 (m, 2H), 1.44 (br s, 9H), 1.43-1.38 (m, 1H), 1.36-1.21 (m, 2H), 1.17 (s, 3H), 1.17 (s, 3H), 1.16-1.09 (m, 1H), 1.04 (qd, J=13.0, 4.3 Hz, 1H), 0.96 (td, J=11.6, 4.0 Hz, 1H), 0.72 (s, 3H). ¹³C NMR (151 MHZ, CDCl₃) δ 199.6, 171.2, 164.4, 156.5, 153.7, 124.0, 79.4, 56.9, 56.7, 55.5, 53.9, 50.1, 47.1, 46.1, 44.2, 38.7, 38.6, 35.9, 35.8, 34.8, 34.1, 32.9, 32.0, 28.6, 24.3, 23.9, 23.2, 21.1, 17.5, 16.9, 13.7. HRMS (APCI+) [M+H]⁺ calc. for C₃₃H₅₂N₃O₅, 570.3907, observed, 570.39009.

### AO. Preparation of tert-butyl(2S)-2-(((((((E)-1-((8S,9S,10R,13S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)(methyl)amino)methyl)piperidine-1-carboxylate (311)

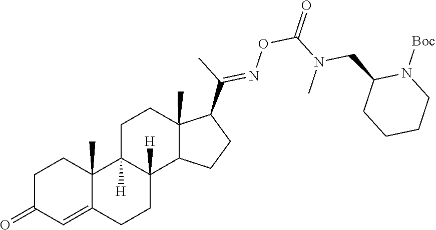

Prepared according to General Procedure A using amine 15b as the amine nucleophile. The product was in the form of a white solid (0.63 mmol, quant. yield). ¹H NMR (600 MHZ, CDCl₃) δ 5.72 (s, 1H), 4.48 (brs, 1H), 4.07-3.70 (m, 1H), 3.62-3.17 (m, 1H), 2.96 (s, 3H), 3.08-2.74 (m, 1H), 2.48-2.19 (m, 3H), 2.05-1.97 (m, 2H), 1.95-1.90 (m, 4H), 1.90-1.83 (m, 1H), 1.80-1.67 (m, 5H), 1.65-1.52 (m, 7H), 1.47-1.38 (m, 11H), 1.37-1.25 (m, 3H), 1.18 (s, 3H), 1.17-1.11 (m, 1H), 1.05 (qd, J=13.1, 4.2 Hz, 1H), 1.00-0.93 (m, 1H), 0.72 (s, 3H). ¹³C NMR (151 MHz, CDCl₃) δ 199.5, 171.1, 164.2, 154.9, 154.9, 123.9, 79.3, 56.8, 55.4, 53.8, 48.2, 48.0, 47.0, 44.0, 40.0, 38.7, 35.7, 35.7, 34.0, 32.8, 31.9, 28.4, 26.1, 25.4, 24.2, 23.1, 21.0, 19.3, 17.4, 16.8, 16.8, 13.5. HRMS (APCI+) [M+H]⁺ calc. for C₃₄H₅₃N₃O₅, 584.4058, observed, 584.4040.

### AP. Preparation of tert-butyl(2S,4R)-4-((tert-butyldimethylsilyl)oxy)-2-(((((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)(methyl)amino)methyl)pyrrolidine-1-carboxylate (31m)

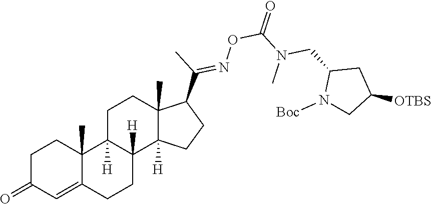

Prepared according to General Procedure A using amine 26 as the amine nucleophile. The product was in the form of a white solid (507 mg, 0.724 mmol, 85%). ¹H NMR (600 MHZ, CDCl₃) δ 5.72 (s, 1H), 4.34 (m, 1H), 4.10-4.04 (m, 1H), 3.59-3.27 (m, 4H), 2.98 (brs, J=10.9 Hz, 4H), 2.48-2.19 (m, 6H), 2.06-1.97 (m, 1H), 1.95-1.84 (m, 6H), 1.81-1.66 (m, 3H), 1.62-1.51 (m, 2H), 1.50-1.41 (m, 10H), 1.37-1.26 (m, 2H), 1.18 (s, 3H), 1.16-1.12 (m, 1H), 1.10-1.01 (m, 1H), 1.00-0.92 (m, 1H), 0.85 (s, 9H), 0.73 (s, 3H), 0.04 (s, 6H). ¹³C NMR (151 MHZ, CDCl₃) δ 199.6, 171.2, 164.6, 155.3, 155.0, 124.0, 79.5, 70.4, 56.9, 55.5, 55.0, 54.6, 53.9, 52.3, 51.4, 44.2, 38.7, 38.6, 38.3, 35.9, 35.9, 34.8, 34.1, 32.9, 32.0, 28.6, 25.9, 24.3, 23.2, 21.1, 18.1, 17.5, 17.0, 13.6, −4.7. HRMS (APCI+) [M+H]⁺ calc. for C₃₉H₆₆N₃O₆Si, 700.471535, observed, 700.469400.

### AQ. Preparation of tert-butyl(2S)-2-(((((((E)-1-((8S,9S,10R,13S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)oxy)methyl)pyrrolidine-1-carboxylate (31n)

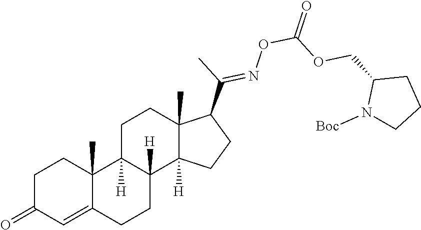

Prepared according to General Procedure B using N-Boc-L-prolinol as the alcohol. The product was in the form of a colorless oil (154 mg, 0.277 mmol, 55% yield). ¹H NMR (600 MHZ, CDCl₃) δ 5.75-5.71 (m, 1H), 4.34-4.00 (m, 3H), 3.37 (s, 2H), 2.46-2.31 (m, 4H), 2.31-2.22 (m, 2H), 2.05-1.99 (m, 1H), 2.00-1.81 (m, 9H), 1.80-1.67 (m, 3H), 1.63-1.51 (m, 2H), 1.46 (s, 10H), 1.37-1.23 (m, 2H), 1.18 (s, 4H), 1.10-1.01 (m, 1H), 1.01-0.94 (m, 1H), 0.72 (s, 3H). HRMS (ESI+) [M+H]⁺ calc. for C₃₃H₄₅O₂N₆, 557.35985, observed, 557.35847.C₃₃H₄₅O₂N₆, 557.35985, observed, 557.35847.

### AR. Preparation of tert-butyl(2-((((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)(methyl)amino)ethyl)(2-fluoroethyl)carbamate (310)

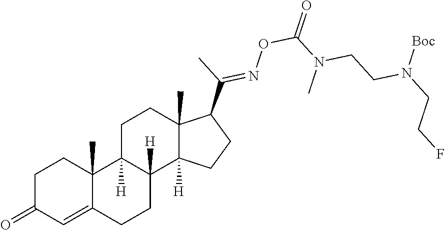

Prepared according to General Procedure A using amine 11b as the amine nucleophile. Rf=0.44 (30% ethyl acetate/DCM). The product was in the form of a white solid (340 mg, 0.591 mmol, 84% yield). ¹H NMR (400 MHZ, DMSO) δ 5.63 (s, 1H), 4.59-4.52 (m, 1H), 4.47-4.41 (m, 1H), 3.53-3.39 (m, 4H), 2.94-2.81 (m, 3H), 2.45-2.31 (m, 3H), 2.29-2.10 (m, 3H), 2.00-1.93 (m, 1H), 1.90 (s, 3H), 1.87-1.76 (m, 2H), 1.69-1.47 (m, 5H), 1.37 (s, 11H), 1.38-1.13 (m, 4H), 1.14 (s, 3H), 1.05-0.88 (m, 2H), 0.63 (s, 3H); ¹³C NMR (101 MHZ, DMSO) § 198.0, 170.9, 163.9, 154.6, 153.7, 123.2, 83.0, 81.3, 79.0, 55.9, 54.7, 53.2, 46.8, 45.3, 44.9, 43.4, 38.2, 37.8, 35.1, 35.1, 33.6, 32.0, 31.6, 27.9, 23.7, 22.5, 20.6, 16.9, 16.7, 13.1. HRMS (APCI+) [M+H]⁺ calc. for C₃₂H₅₁FN₃O₅, 576.38073, observed, 576,38096.

### AS. Preparation of (8S,9S,10R,13S,14S,17S)-17-((E)-1-((((2-aminoethyl)carbamoyl)oxy)imino)ethyl)-10,13-dimethyl-1,2,6,7,8,9,10,11,12,13,14,15,16,17-tetradecahydro-3H-cyclopenta[a]phenanthren-3-one hydrochloride (32a)

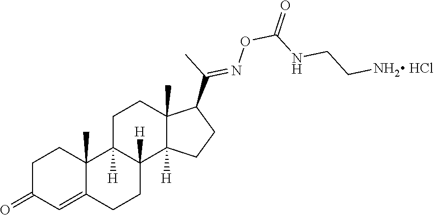

Prepared according to General Procedure C. The product was in the form of a white solid (73 mg, 0.1615 mmol, 66.623% yield). ¹H NMR (400 MHZ, CDCl₃) δ 8.25 (brs, 3H), 7.04 (brs, 1H), 5.73 (s, 1H), 3.69 (brs, 2H), 3.29 (brs, 2H), 2.99 (s, 1H), 2.51-2.12 (m, 5H), 2.07-2.02 (m, 1H), 1.98 (s, 3H), 1.94-1.81 (m, 2H), 1.75-1.48 (m, 5H), 1.48-1.21 (m, 3H), 1.18 (s, 4H), 1.14-0.92 (m, 2H), 0.67 (s, 3H). ¹³C NMR (151 MHZ, CDCl₃) δ 199.4, 170.8, 165.0, 157.1, 124.0, 63.5, 56.8, 56.0, 55.4, 53.7, 44.2, 40.2, 38.8, 38.6, 35.7, 33.9, 32.8, 31.8, 24.1, 23.0, 21.0, 17.4, 17.3, 13.4. HRMS (APCI+) [M+H]⁺ calc. for C₂₄H₃₈O₃N₃, 416.29077, observed, 416.29063.

### AT. Preparation of (E)-9-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)-5-methyl-7-oxa-2,5,8-triazadec-8-en-6-one hydrochloride (32b)

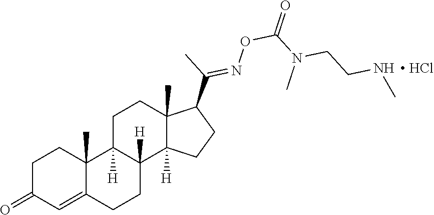

Prepared according to General Procedure C. The product was in the form of a white solid (109 mg, 0.2270 mmol, 68.585% yield). ¹H NMR (600 MHZ, CDCl₃) δ 9.72-9.45 (m, 2H), 5.73 (s, 1H), 3.87-3.65 (m, 2H), 3.24 (brs, 2H), 3.04 (s, 3H), 2.75 (brs, 3H), 2.46-2.31 (m, 4H), 2.31-2.25 (m, 1H), 2.22-2.16 (m, 1H), 2.05-2.00 (m, 1H), 1.97 (s, 3H), 1.94-1.90 (m, 1H), 1.89-1.83 (m, 1H), 1.82-1.66 (m, 3H), 1.64-1.51 (m, 2H), 1.48-1.39 (m, 1H), 1.32 (s, 2H), 1.18 (s, 3H), 1.17-1.14 (m, 1H), 1.11-1.03 (m, 1H), 0.98 (td, J=11.6, 4.1 Hz, 1H), 0.71 (s, 3H). ¹³C NMR (151 MHZ, CDCl₃) δ 199.6, 171.1, 166.1, 156.3, 124.1, 56.8, 55.4, 53.8, 47.6, 46.4, 45.2, 44.4, 38.7, 38.5, 35.8, 35.2, 34.1, 33.6, 32.9, 32.0, 24.3, 23.2, 21.1, 17.5, 17.2, 13.6. HRMS (APCI+) [M+H]⁺ calc. for C₂₆H₄₂O₃N₃, 444.32207, observed, 444.3222.

### AU. Preparation of 2-((((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)(2-hydroxyethyl)amino)-N-methylethan-1-aminium chloride (32c)

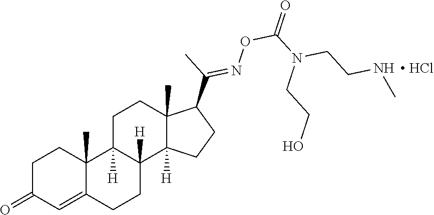

Prepared according to General Procedure C. Purified by flash chromatography (0-20% MeOH/DCM). The product was in the form of a white solid (64 mg, 0.125 mmol, 72% yield). ¹H NMR (600 MHZ, CDCl₃) δ 9.65-9.25 (m, 2H), 5.73 (s, 1H), 3.88-3.83 (m, 2H), 3.82-3.76 (m, 3H), 3.50-3.45 (m, 2H), 3.39-3.32 (m, 2H), 2.72 (brs, 3H), 2.46-2.31 (m, 4H), 2.30-2.16 (m, 2H), 2.05-1.98 (m, 1H), 1.93 (s, 3H), 1.92-1.83 (m, 3H), 1.79-1.66 (m, 3H), 1.63-1.51 (m, 2H), 1.43 (qd, J=13.0, 3.8 Hz, 1H), 1.35-1.23 (m, 2H), 1.18 (s, 3H), 1.17-1.14 (m, 1H), 1.05 (qd, J=13.0, 3.8 Hz, 1H), 0.97 (td, J=11.6, 4.1 Hz, 1H), 0.71 (s, 3H). ¹³C NMR (151 MHZ, CDCl₃) δ 199.7, 171.2, 166.2, 156.0, 124.1, 60.3, 56.8, 55.5, 53.8, 53.0, 49.4, 48.1, 44.4, 38.7, 38.5, 35.8, 35.8, 34.0, 33.7, 32.9, 32.0, 24.3, 23.2, 21.1, 17.5, 17.3, 13.6. HRMS (APCI+) [M+H]⁺ calc. for C₂₇H₄₅N₃O₄, 474.33263, observed, 474.33246.

### AV. Preparation of 2-((((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)(methyl)amino)-N-(2-hydroxyethyl)ethan-1-aminium chloride (32d)

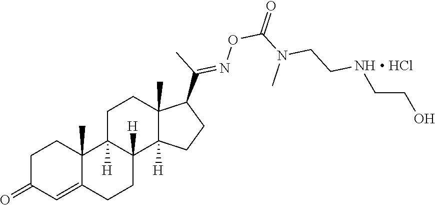

Prepared according to General Procedure C. Purified by flash chromatography (0-20% MeOH/DCM). The product was in the form of a white solid (112 mg, 0.219 mmol, 75% yield). ¹H NMR (600 MHZ, CDCl₃) δ 9.65-9.25 (m, 2H), 5.72 (s, 1H), 3.95-3.90 (m, 2H), 3.83-3.61 (m, 3H), 3.35-3.28 (m, 2H), 3.25-3.18 (s, 2H), 3.03 (s, 3H), 2.45-2.16 (m, 7H), 2.04-1.98 (m, 1H), 1.96 (s, 3H), 1.93-1.88 (m, 1H), 1.87-1.83 (m, 1H), 1.79-1.66 (m, 3H), 1.63-1.50 (m, 2H), 1.42 (qd, J=12.9, 3.7 Hz, 1H), 1.35-1.23 (m, 2H), 1.18 (s, 3H), 1.16-1.12 (m, 1H), 1.05 (qd, J=13.0, 3.8 Hz, 1H), 0.97 (td, J=11.6, 4.1 Hz, 1H), 0.70 (s, 3H). ¹³C NMR (151 MHZ, CDCl₃) δ 199.7, 171.2, 166.2, 156.4, 124.0, 57.3, 56.8, 55.4, 53.8, 50.5, 46.3, 46.0, 44.3, 38.7, 38.5, 35.8, 35.8, 34.9, 34.0, 32.9, 32.0, 24.3, 23.2, 21.1, 17.5, 17.2, 13.6. HRMS (APCI+) [M+H]⁺ calc. for C₂₇H₄₅N₃O₄, 474.33263, observed, 474.33254.

### AW. Preparation of (8S,9S,10R,13S,14S,17S)-17-((E)-1-((((3-aminopropyl)carbamoyl)oxy)imino)ethyl)-10,13-dimethyl-1,2,6,7,8,9,10,11,12,13,14,15,16,17-tetradecahydro-3H-cyclopenta[a]phenanthren-3-one hydrochloride (32e)

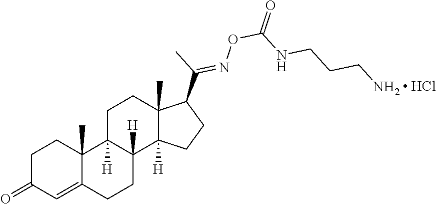

Prepared according to General Procedure C. The product was in the form of a white solid (109 mg, 0.2339 mmol, 78.409% yield). ¹H NMR (400 MHZ, CDCl₃) δ 8.30 (s, 3H), 6.72 (t, J=6.3 Hz, 1H), 5.75 (d, J=1.6 Hz, 1H), 3.48 (q, J=6.9 Hz, 2H), 3.14 (s, 2H), 2.46-2.27 (m, 5H), 2.18 (d, J=10.1 Hz, 4H), 2.12-2.05 (m, 1H), 2.02 (s, 4H), 1.91 (tt, J=15.8, 2.6 Hz, 2H), 1.83-1.70 (m, 2H), 1.71-1.52 (m, 2H), 1.52-1.25 (m, 2H), 1.25-1.15 (m, 4H), 1.15-0.95 (m, 2H), 0.70 (s, 3H). ¹³C NMR (151 MHZ, CDCl₃) δ 199.8, 171.3, 164.9, 157.3, 124.0, 56.9, 55.6, 53.8, 44.3, 38.7, 38.7, 37.7, 37.2, 35.9, 35.8, 34.0, 32.9, 32.0, 27.8, 24.2, 23.2, 21.1, 17.5, 17.4, 13.6. HRMS (APCI+) [M+H]⁺ calc. for C₂₅H₄₀O₃N₃, 430.30642, observed, 430.30637.

### AX. Preparation of (8S,9S,10R,13S,14S,17S)-17-((E)-1-((((2-aminoethoxy)carbonyl)oxy)imino)ethyl)-10,13-dimethyl-1,2,6,7,8,9,10,11,12,13,14,15,16,17-tetradecahydro-3H-cyclopenta[a]phenanthren-3-one hydrochloride (32f)

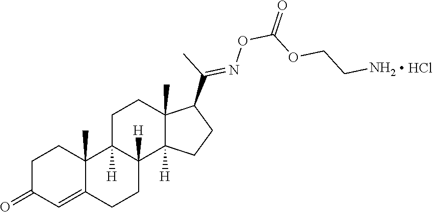

Prepared according to General Procedure C. The product was in the form of a white solid (55 mg, 0.1214 mmol, 41.819% yield). ¹H NMR (400 MHZ, CDCl₃) δ 8.43 (s, 3H), 5.73 (d, J=1.6 Hz, 1H), 4.71-4.43 (m, 2H), 3.43 (s, 2H), 2.85 (brs, 2H), 2.48-2.13 (m, 6H), 2.07-2.02 (m, 1H), 1.99 (s, 3H), 1.95-1.81 (m, 2H), 1.81-1.64 (m, 2H), 1.64-1.21 (m, 4H), 1.18 (s, 3H), 1.15-0.90 (m, 3H), 0.70 (s, 3H). ¹³C NMR (101 MHz, CDCl₃) δ 199.5, 170.9, 167.6, 153.9, 124.0, 77.2, 64.1, 56.6, 55.4, 53.7, 44.2, 39.3, 38.6, 38.4, 35.7, 33.9, 32.8, 31.8, 24.1, 23.0, 21.0, 17.4, 17.1, 13.4. HRMS (APCI+) [M+H]⁺ calc. for C₂₄H₃₇N₂O₄, 417.2753, observed, 417.2734.

### AY. Preparation of (8S,9S,10R,13S,17S)-10,13-dimethyl-17-((E)-1-((((((S)-pyrrolidin-2-yl)methyl)carbamoyl)oxy)imino)ethyl)-1,2,6,7,8,9,10,11,12,13,14,15,16,17-tetradecahydro-3H-cyclopenta[a]phenanthren-3-one hydrochloride (32g)

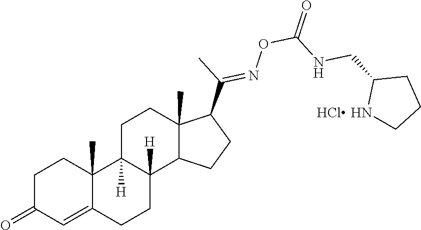

Synthesized according to General Procedure C and purified by flash chromatography (0-15% methanol in DCM). The product was in the form of a white solid (56 mg, 0.1138 mmol, 21% yield). ¹H NMR (600 MHZ, CDCl₃) δ 9.99 (brs, 1H), 9.46 (brs, 1H), 7.34 (t, J=6.3 Hz, 1H), 5.72 (s, 1H), 3.93 (brs, 1H), 3.78-3.72 (m, 1H), 3.70-3.63 (m, 1H), 3.39-3.35 (m, 1H), 3.32-3.27 (m, 1H), 2.46-2.24 (m, 6H), 2.21-2.13 (m, 1H), 2.11-2.05 (m, 1H), 2.05-1.83 (m, 8H), 1.77-1.66 (m, 3H), 1.56 (dtd, J=33.2, 12.3, 3.6 Hz, 2H), 1.42 (qd, J=12.9, 3.6 Hz, 1H), 1.37-1.23 (m, 2H), 1.17 (s, 4H), 1.10-1.00 (m, 1H), 1.00-0.94 (m, 1H), 0.69 (s, 3H). ¹³C NMR (151 MHZ, CDCl₃) δ 199.6, 171.1, 165.1, 157.4, 124.1, 60.3, 56.9, 55.5, 53.8, 45.5, 44.3, 41.9, 38.7, 38.6, 35.9, 35.8, 34.1, 32.9, 32.0, 27.7, 24.4, 24.3, 23.2, 21.1, 17.5, 17.4, 13.6. HRMS (ESI+) [M+H]⁺ calc. for C₂₇H₄₂O₃N₃, 456.32207, observed, 456.32192.

### AZ. Preparation of ((2S)-1-(((((E)-1-((8S,9S,10R,13S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)pyrrolidin-2-yl) methanaminium chloride (32h)

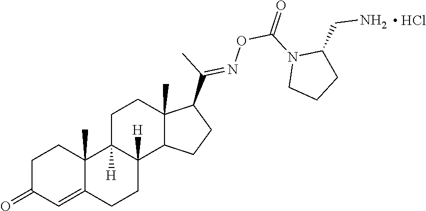

Prepared according to General Procedure C. Purified by flash chromatography (0-20% MeOH/DCM). The product was in the form of a white solid (423 mg, 0.860 mmol, 92% yield). ¹H NMR (600 MHZ, CDCl₃) δ 8.60 (s, 3H), 5.72 (s, 1H), 4.28-4.20 (m, 1H), 3.58-3.29 (m, 4H), 3.16-3.07 (m, 1H), 2.46-2.15 (m, 7H), 2.04-1.99 (m, 1H), 1.95 (s, 3H), 1.93-1.83 (m, 3H), 1.80-1.65 (m, 4H), 1.64-1.51 (m, 2H), 1.43 (qd, J=13.0, 3.8 Hz, 1H), 1.35-1.23 (m, 2H), 1.18 (s, 3H), 1.17-1.12 (m, 1H), 1.05 (qd, J=12.9, 4.2 Hz, 1H), 0.97 (td, J=11.6, 4.0 Hz, 1H), 0.72 (s, 3H). ¹³C NMR (151 MHZ, CDCl₃) δ 199.7, 171.3, 166.2, 156.3, 124.0, 56.9, 56.7, 55.4, 53.8, 47.2, 44.9, 44.3, 38.7, 38.5, 35.8 (2C), 34.0, 32.9, 32.0, 30.3, 24.3, 24.1, 23.2, 21.1, 17.5, 17.1, 13.6. HRMS (APCI+) [M+H]⁺ calc. for C₂₇H₄₂N₃O₃, 456. 32207, observed, 456.32179.

### BA. Preparation of (8S,9S,10R,13S,17S)-10,13-dimethyl-17-((E)-1-((((((S)-piperidin-2-yl)methyl)carbamoyl)oxy)imino)ethyl)-1,2,6,7,8,9,10,11,12,13,14,15,16,17-tetradecahydro-3H-cyclopenta[a]phenanthren-3-one hydrochloride (32i)

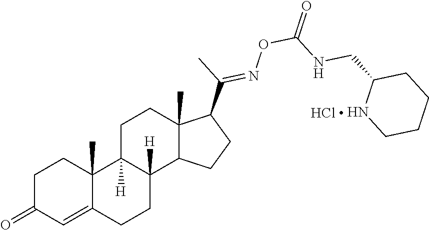

Prepared according to General Procedure C and purified by flash chromatography (0-15% methanol in DCM). The product was in the form of a white solid (205 mg, 0.405 mmol, 75% yield). ¹H NMR (600 MHZ, CDCl₃) δ 9.47-9.37 (m, 1H), 9.29 (s, 1H), 7.19-7.11 (m, 1H), 5.72 (s, 1H), 3.73-3.58 (m, 2H), 3.50 (d, J=12.4 Hz, 1H), 3.23 (s, 1H), 2.84 (q, J=10.4 Hz, 1H), 2.45-2.21 (m, 7H), 2.01 (ddt, J=10.6, 5.5, 2.8 Hz, 1H), 1.97 (s, 3H), 1.95-1.65 (m, 9H), 1.61-1.38 (m, 4H), 1.36-1.22 (m, 2H), 1.17 (s, 3H), 1.13 (d, J=11.7 Hz, 1H), 1.08-1.00 (m, 1H), 0.99-0.93 (m, 1H), 0.68 (s, 3H), 0.67* (s, 3H). ¹³C NMR (151 MHZ, CDCl₃) δ 199.6, 171.1, 164.7, 156.8, 124.1, 57.8, 57.7, 56.9, 55.5, 53.8, 45.1, 44.2, 43.6, 43.5, 38.7, 35.9, 34.1, 32.9, 32.0, 26.3, 24.2, 23.1, 23.1, 22.4, 22.1, 21.1, 17.5, 13.5. HRMS (APCI+) [M+H]⁺ calc. for C₂₈H₄₄O₃N₃, 470.33772, observed, 470.33776.

### BB. Preparation of (8S,9S,10R,13S,17S)-10,13-dimethyl-17-((E)-1-(((methyl(((S)-pyrrolidin-2-yl)methyl)carbamoyl)oxy)imino)ethyl)-1,2,6,7,8,9,10,11,12,13,14,15,16,17-tetradecahydro-3H-cyclopenta[a]phenanthren-3-one hydrochloride (32j)

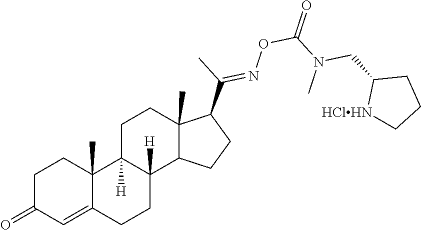

Prepared according to General Procedure C and purified by flash chromatography (0-15% methanol in DCM). The product was in the form of a white solid (165 mg, 0.326 mmol, 69% yield). ¹H NMR (600 MHZ, CDCl₃) δ 11.37 (brs, 1H), 8.18 (brs, 1H), 5.73 (s, 1H), 4.17 (brs, 1H), 3.84 (dd, J=15.2, 9.4 Hz, 1H), 3.59-3.45 (m, 1H), 3.38-3.20 (m, 2H), 3.10 (s, 3H), 2.47-2.29 (m, 4H), 2.28-2.15 (m, 3H), 2.13-2.03 (m, 2H), 2.03-1.94 (m, 4H), 1.91 (d, J=8.8 Hz, 1H), 1.87-1.65 (m, 5H), 1.61-1.49 (m, 2H), 1.42 (qd, J=13.0, 3.5 Hz, 1H), 1.35-1.21 (m, 2H), 1.17 (s, 3H), 1.16-1.12 (m, 1H), 1.08-1.00 (m, 1H), 0.96 (td, J=11.5, 3.9 Hz, 1H), 0.71 (s, 3H). ¹³C NMR (151 MHZ, CDCl₃) δ 199.6, 171.1, 166.4, 157.2, 124.0, 59.4, 56.8, 55.4, 53.8, 51.2, 45.2, 44.3, 38.7, 38.5, 36.1, 35.8, 34.0, 32.9, 31.9, 28.1, 24.2, 23.7, 23.2, 21.1, 17.5, 17.3, 13.7. HRMS (APCI+) [M+H]⁺ calc. for C₂₈H₄₄N₃O₃, 470.3382, observed, 470.3361.

### BC. Preparation of 1-((2S)-1-(((((E)-1-((8S,9S,10R,13S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)pyrrolidin-2-yl)-N-methylmethanaminium chloride (32k)

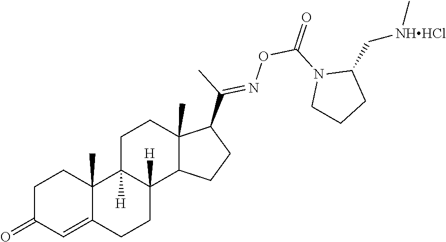

Prepared according to General Procedure C and purified by flash chromatography (0-20% MeOH/DCM). The product was in the form of a white solid (231 mg, 0.456 mmol, 93% yield). ¹H NMR (600 MHZ, CDCl₃) δ 10.16 (s, 1H), 9.48 (s, 1H), 5.71 (s, 1H), 4.34-4.26 (m, 1H), 3.59-3.52 (m, 1H), 3.50-3.43 (m, 1H), 3.35-3.27 (m, 1H), 3.22-3.02 (m, 3H), 2.73 (s, 3H), 2.45-2.15 (m, 7H), 2.03-1.98 (m, 1H), 1.95 (s, 3H), 1.94-1.83 (m, 3H), 1.80-1.65 (m, 4H), 1.62-1.50 (m, 2H), 1.42 (qd, J=13.0, 3.8 Hz, 1H), 1.35-1.22 (m, 2H), 1.18 (s, 3H), 1.17-1.11 (m, 1H), 1.04 (qd, J=12.9, 4.2 Hz, 1H), 0.96 (td, J=11.6, 4.0 Hz, 1H), 0.72 (s, 3H). ¹³C NMR (151 MHz, CDCl₃) δ 199.6, 171.1, 166.4, 156.2, 124.0, 56.6, 55.8, 55.4, 54.3, 53.8, 47.2, 44.4, 38.7, 38.4, 35.8 (2C), 34.0, 33.6, 32.9, 31.9, 30.4, 24.3, 24.0, 23.2, 21.0, 17.5, 17.0, 13.6. HRMS (APCI+) [M+H]⁺ calc. for C₂₈H₄₄N₃O₃, 470.3377, observed, 470.3376.

### BD. Preparation of (8S,9S,10R,13S,17S)-10,13-dimethyl-17-((E)-1-(((methyl(((S)-piperidin-2-yl)methyl)carbamoyl)oxy)imino)ethyl)-1,2,6,7,8,9,10,11,12,13,14,15,16,17-tetradecahydro-3H-cyclopenta[a]phenanthren-3-one hydrochloride (321)

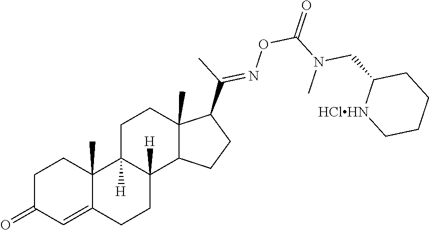

Prepared according to General Procedure C and purified by flash chromatography (0-15% methanol in DCM). The product was in the form of a light brown solid (121 mg, 0.23 mmol, 49% yield). ¹H NMR (600 MHZ, CDCl₃) δ 5.72 (s, 1H), 3.84-3.74 (m, 1H), 3.59-3.35 (m, 3H), 3.05 (s, 3H), 2.93-2.78 (m, 1H), 2.44-2.17 (m, 6H), 2.04-1.66 (m, 15H), 1.62-1.38 (m, 4H), 1.35-1.22 (m, 2H), 1.17 (s, 3H), 1.16-1.13 (m, 1H), 1.10-1.00 (m, 1H), 0.99-0.94 (m, 1H), 0.71 (s, 3H), 0.70* (s, 3H). ¹³C NMR (151 MHZ, CDCl₃) δ 199.6, 171.2, 165.8, 156.0, 124.0, 56.8, 55.5, 55.4, 55.4, 53.8, 52.4, 44.9, 44.3, 38.7, 38.6, 36.2, 35.8, 34.1, 32.9, 32.0, 26.4, 24.3, 23.2, 22.2, 21.1, 17.5, 17.2, 17.2, 13.6. HRMS (APCI+) [M+H]⁺ calc. for C₂₉H₄₆N₃O₃, 484.3539, observed, 484.3516.

### BE. Preparation of (8S,9S,10R,13S,14S,17S)-17-((E)-1-((((((2S,4R)-4-hydroxypyrrolidin-2-yl)methyl)(methyl)carbamoyl)oxy)imino)ethyl)-10,13-dimethyl-1,2,6,7,8,9,10,11,12,13,14,15,16,17-tetradecahydro-3H-cyclopenta[a]phenanthren-3-one 2,2,2-trifluoroacetate (32m)

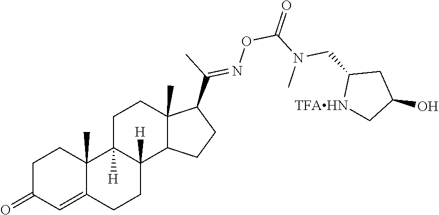

Prepared according to General Procedure C with the following exceptions. After deprotection of the TBS and Boc protecting groups with TFA, the crude oil was purified by flash chromatography to afford the title compound as a trifluoroacetate salt in the form of a white solid (235 mg, 0.39 mmol, 54% yield). ¹H NMR (600 MHZ, CDCl₃) δ 10.96 (brs, 1H), 8.54 (brs, 1H), 5.73 (s, 1H), 4.59 (t, J=4.2 Hz, 1H), 4.32 (s, 1H), 3.81 (dd, J=15.4, 8.6 Hz, 1H), 3.48-3.37 (m, 3H), 3.21 (brs, 2H), 3.04 (s, 3H), 2.46-2.12 (m, 7H), 2.06-1.99 (m, 1H), 1.97 (s, 3H), 1.93-1.83 (m, 3H), 1.80-1.66 (m, 3H), 1.63-1.50 (m, 2H), 1.43 (qd, J=13.0, 3.8 Hz, 1H), 1.36-1.23 (m, 2H), 1.18 (s, 3H), 1.17-1.13 (m, 1H), 1.10-1.01 (m, 1H), 0.97 (td, J=11.6, 4.1 Hz, 1H), 0.72 (s, 3H). ¹³C NMR (151 MHZ, CDCl₃) δ 199.7, 171.2, 166.9, 158.0, 124.1, 69.9, 58.6, 56.7, 55.4, 53.8, 53.2, 51.4, 44.4, 38.7, 38.4, 37.4, 36.0, 35.8, 34.1, 32.9, 32.0, 24.3, 23.2, 21.1, 17.5, 17.0, 13.6. HRMS (APCI+) [M+H]⁺ calc. for C₂₈H₄₄N₃O₄, 486.3332, observed, 486.3309.

### BF. Preparation of (8S,9S,10R,13S,17S)-10,13-dimethyl-17-((E)-1-((((((S)-pyrrolidin-2-yl) methoxy)carbonyl)oxy)imino)ethyl)-1,2,6,7,8,9,10,11,12,13,14,15,16,17-tetradecahydro-3H-cyclopenta[a]phenanthren-3-one hydrochloride (32n)

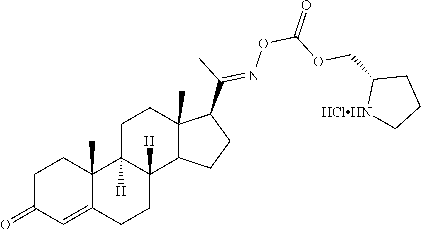

Prepared according to General Procedure C. The product was in the form of a white solid (78 mg, 0.158 mmol, 63% yield). ¹H NMR (600 MHZ, CDCl₃) δ 10.27 (s, 1H), 9.64 (s, 1H), 5.72 (s, 1H), 4.63 (dd, J=12.1, 3.9 Hz, 1H), 4.57 (dd, J=12.0, 5.7 Hz, 1H), 3.99 (brs, 1H), 3.45 (brs, J=10.1 Hz, 2H), 2.44-2.17 (m, 7H), 2.16-2.10 (m, 1H), 2.07-2.00 (m, 2H), 1.99 (s, 3H), 1.95-1.82 (m, 3H), 1.80-1.64 (m, 3H), 1.63-1.50 (m, 2H), 1.48-1.37 (m, 1H), 1.36-1.23 (m, 2H), 1.17 (s, 3H), 1.15-1.12 (m, 1H), 1.09-1.02 (m, 1H), 0.97 (td, J=11.6, 4.1 Hz, 1H), 0.71 (s, 3H). ¹³C NMR (151 MHZ, CDCl₃) δ 199.6, 171.1, 167.5, 153.6, 124.1, 66.0, 58.1, 56.7, 55.5, 53.8, 45.7, 44.3, 38.7, 38.5, 35.8, 35.8, 34.0, 32.9, 32.0, 27.2, 24.2, 23.8, 23.1, 21.1, 17.5, 17.3, 13.6. HRMS (APCI+) [M+H]⁺ calc. for C₂₇H₄₁O₄N₂, 457.30608, observed, 457.30593. 431.29018.

### BG. Preparation of (E)-2-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)-11-fluoro-6-methyl-4-oxa-3,6,9-triazaundec-2-en-5-one 2,2,2-trifluoroacetate (320)

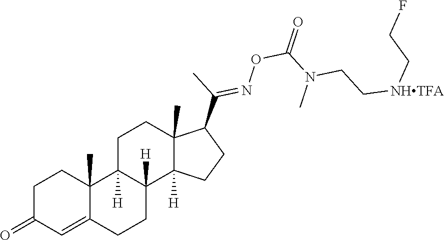

Prepared according to Procedure C. The product was in the form of a white solid (165 mg, 0.288 mmol, 54% yield). ¹H NMR (400 MHZ, CDCl₃) δ 9.82 (br s, 2H), 5.72 (s, 1H), 4.95 (br s, 1H), 4.83 (br s, 1H), 3.90-3.67 (m, 2H), 3.53-3.29 (m, 4H), 3.04 (s, 3H), 2.80 (br s, 2H), 2.47-2.14 (m, 6H), 1.97 (s, 3H), 1.95-1.81 (m, 2H), 1.78-1.64 (m, 2H), 1.55 (m, 2H), 1.47-1.27 (m, 3H), 1.17 (s, 3H), 1.12-0.92 (m, 3H), 0.70 (s, 3H); ¹³C NMR (101 MHZ, CDCl₃) δ 199.7, 171.2, 166.2, 156.3, 124.0, 80.5, 78.8, 77.4, 56.8, 55.4, 53.8, 47.7, 46.0, 44.3, 38.7, 38.5, 35.8, 34.9, 34.0, 32.9, 31.9, 24.3, 23.2, 21.1, 17.5, 17.2, 13.6. HRMS (APCI+) [M+H]⁺ calc. for C₂₇H₄₃FN₃O₃, 476.3283, observed, 477.32842.

### BH. Preparation of (8S,9S,10R,13S,14S,17S)-17-((E)-1-((((2-hydroxyethyl)(methyl)carbamoyl)oxy)imino)ethyl)-10,13-dimethyl-1,2,6,7,8,9,10,11,12,13,14,15,16,17-tetradecahydro-3H-cyclopenta[a]phenanthren-3-one (33)

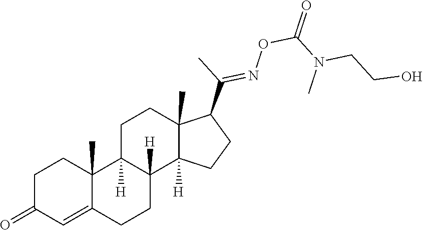

Prepared according to General Procedure A using N-methylethanolamine as the amine nucleophile. The product was in the form of a white solid (220 mg, 0.5109 mmol, 84.233% yield). ¹H NMR (600 MHZ, CDCl₃) δ 5.7 (s, 1H), 3.8 (t, J=5.1 Hz, 2H), 3.5 (s, 2H), 3.0 (s, 3H), 2.6 (s, 1H), 2.5-2.2 (m, 6H), 2.0 (d, J=2.0 Hz, 1H), 1.9 (s, 4H), 1.9 (d, J=12.9 Hz, 1H), 1.7 (d, J=19.2 Hz, 3H), 1.6 (s, 2H), 1.4 (s, 1H), 1.3 (d, J=5.9 Hz, 2H), 1.2 (s, 3H), 1.1 (s, 1H), 1.1 (s, 1H), 1.0-0.9 (m, 1H), 0.7 (s, 3H). ¹³C NMR (151 MHZ, CDCl₃) δ 199.6, 171.3, 165.0, 156.4, 124.0, 61.5, 56.9, 55.5, 53.9, 52.5, 44.2, 38.7, 38.6, 35.9, 35.8, 35.6, 34.1, 32.9, 32.0, 24.3, 23.2, 21.1, 17.5, 17.1, 13.6. HRMS (ESI+) [M+H]⁺ calc. for C₂₅H₃₉O₄N₂, 431.29043, observed, 431.29018.

### BI. Preparation of (E)-9-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)-2-methyl-7-oxa-2,5,8-triazadec-8-en-6-one hydrochloride (34)

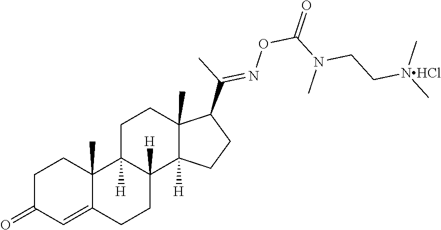

Prepared according to General Procedures A and C using N,N-dimethyl-1,2-ethanediamine as the amine nucleophile with the following exceptions. The title compound was purified as the amine free base by flash chromatography (100% DCM-100% (DCM:MeOH:NH₃OH 97:2.5:0.5). To the amine free base was added HCl (2 M in ether, 1 equiv.), and concentration in vacuo afforded the title compound as a white solid (220 mg, 0.4583 mmol, 66.742% yield). ¹H NMR (600 MHZ, CDCl₃) δ 7.2 (t, J=6.1 Hz, 1H), 5.7 (s, 1H), 3.8 (q, J=6.1 Hz, 2H), 3.2 (t, J=6.2 Hz, 2H), 2.8 (s, 6H), 2.5-2.2 (m, 6H), 2.0-2.0 (m, 1H), 2.0 (s, 3H), 1.9 (d, J=3.7 Hz, 2H), 1.8-1.6 (m, 3H), 1.6-1.5 (m, 2H), 1.5-1.4 (m, 1H), 1.4-1.2 (m, 2H), 1.2 (s, 3H), 1.2-1.1 (m, 1H), 1.1-1.0 (m, 1H), 1.1-0.9 (m, 1H), 0.7 (s, 3H). ¹³C NMR (151 MHZ, CDCl₃) δ 199.6, 171.1, 164.7, 156.4, 124.0, 58.0, 57.0, 55.6, 53.8, 44.2, 44.2, 38.7, 38.7, 36.8, 35.9, 35.8, 34.1, 32.9, 31.9, 24.2, 23.3, 21.1, 17.5, 17.4, 13.6. HRMS (ESI+) [M+H]⁺ calc. for C₂₆H₄₂O₃N₃; 444.32207, observed, 444.32203.

### BJ. Preparation of (E)-9-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)-2-methyl-5,7-dioxa-2,8-diazadec-8-en-6-one hydrochloride (35)

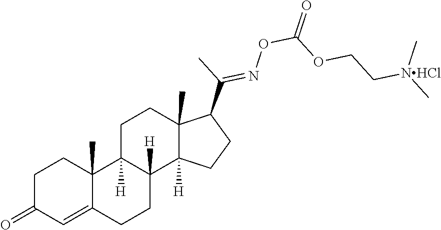

Prepared according to General Procedures B and C using N,N-dimethylethanolamine as the amine nucleophile with the following exceptions. Flash chromatography (100% DCM-100% (88:10:2 DCM:MeOH:NH₃OH)) afforded the amine free base. The free base was then dissolved in ether and cooled in a brine ice bath. Hydrogen chloride (1 mmol, 0.5 mL 2 M in ether) was then added dropwise. The mixture was then concentrated in vacuo and subjected to a second round of flash chromatography (0-10% methanol in DCM) to afford the title compound as a white solid (241 mg, 0.501 mmol, 99.107%). ¹H NMR (600 MHZ, CDCl₃) δ 12.86 (s, 1H), 5.69 (s, 1H), 4.75-4.68 (m, 1H), 3.43 (q, J=4.5 Hz, 2H), 2.89 (d, J=4.4 Hz, 6H), 2.44-2.12 (m, 7H), 1.99 (ddd, J=13.4, 5.1, 3.2 Hz, 1H), 1.95 (s, 3H), 1.90 (dt, J=12.3, 3.4 Hz, 1H), 1.86-1.81 (m, 1H), 1.77-1.64 (m, 3H), 1.62-1.48 (m, 2H), 1.45-1.36 (m, 1H), 1.33-1.20 (m, 2H), 1.15 (s, 3H), 1.14-1.10 (m, 1H), 1.07-0.99 (m, 1H), 0.94 (ddd, J=12.2, 10.6, 4.1 Hz, 1H), 0.68 (s, 3H). ¹³C NMR (151 MHZ, CDCl₃) δ 199.4, 170.9, 167.3, 153.1, 123.9, 62.2, 56.6, 55.9, 55.4, 53.7, 44.1, 43.6, 43.5, 38.6, 38.4, 35.7, 35.7, 33.9, 32.7, 31.8, 24.1, 23.0, 21.0, 17.4, 17.0, 13.4. HRMS (APCI+) [M+H]⁺ calc. for C₂₆H₄₁O₄N₂, 445.30608, observed, 445.30621.

### BK. Preparation of (E)-10-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)-6-methyl-8-oxa-2,6,9-triazaundec-9-en-7-one hydrochloride (39)

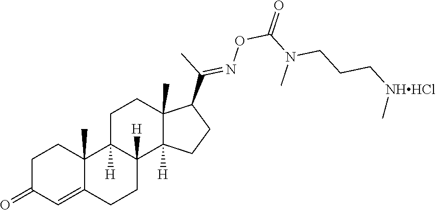

Prepared according to General Procedures A and C. ¹H NMR (600 MHZ, CDCl₃) δ 9.75-9.20 (m, 2H), 5.69 (s, 1H), 3.44 (t, J=6.5 Hz, 2H), 3.02-2.94 (m, 2H), 2.96 (s, 3H), 2.67 (t, J=5.4 Hz, 3H), 2.43-2.10 (m, 8H), 2.03-1.95 (m, 1H), 1.92 (s, 3H), 1.92-1.88 (m, 1H), 1.86-1.81 (m, 1H), 1.78-1.63 (m, 3H), 1.61-1.47 (m, 2H), 1.40 (qd, J=13.1, 3.8 Hz, 1H), 1.34-1.20 (m, 2H), 1.15 (s, 3H), 1.15-1.10 (m, 1H), 1.08-0.98 (m, 1H), 0.97-0.91 (m, 1H), 0.69 (s, 3H). ¹³C NMR (151 MHZ, CDCl₃) δ 199.6, 171.2, 165.5, 155.9, 123.9, 56.7, 55.4, 53.7, 47.1, 46.4, 44.2, 38.6, 38.5, 35.7, 35.7, 34.3, 34.0, 33.4, 32.8, 31.9, 24.5, 24.2, 23.1, 21.0, 17.4, 17.0, 13.6.

### BL. Preparation of (E)-10-((8S,9S,10R,13S,17S)-10,13-dimethyl-3-oxo-2,3,6,7,8,9,10,11,12,13,14,15,16,17-tetradecahydro-1H-cyclopenta[a]phenanthren-17-yl)-4,4,6-trimethyl-8-oxa-2,6,9-triazaundec-9-en-7-one hydrochloride (40)

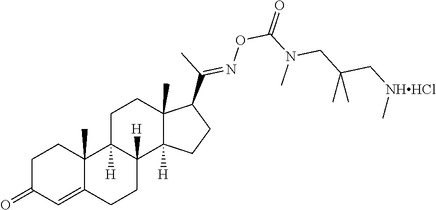

Prepared according to General Procedures A and C with the following exceptions. The free base was dissolved in diethyl ether (0.5 mL) and cooled to 0° C. in a brine ice bath. Hydrochloric acid (1.5 equiv.)(4M in dioxane) was then added dropwise. After stirring for 30 minutes at 0° C., diethyl ether (10 mL) was then added dropwise to the solution at 0° C. resulting in the formation of a white precipitate. The mixture was then filtered over a filter frit and washed with diethyl ether (3× 10 mL) to afford the title compound as a white solid (100 mg, 0.192 mmol, 79% yield). ¹H NMR (400 MHZ, CDCl₃) δ 9.35 (m, 2H), 5.72 (s, 1H), 3.32-3.12 (m, 2H), 3.05 (s, 3H), 2.97-2.84 (m, 1H), 2.84-2.76 (m, 2H), 2.75-2.69 (m, 3H), 2.49-2.13 (m, 6H), 2.07-1.98 (m, 1H), 1.97 (s, 3H), 1.95-1.82 (m, 2H), 1.81-1.68 (m, 3H), 1.67-1.22 (m, 5H), 1.18 (s, 3H), 1.11 (d, J=4.4, 6H), 1.09-0.93 (m, 2H), 0.72 (s, 3H). ¹³C NMR (101 MHZ, CDCl₃) δ 199.7, 171.1, 166.5, 157.7, 124.1, 57.8, 56.7, 55.5, 53.8, 44.3, 38.7, 38.5, 38.3, 36.6, 35.8, 34.7, 34.1, 32.9, 31.9, 24.6, 24.5, 24.3, 23.2, 21.1, 17.5, 17.1, 13.7. HRMS: (APCI+) [M+H]⁺ calc. for C₂₉H₄₈N₃O₃, 486.36902, observed, 486.36995.

### BM. Preparation of (E)-12-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)-1,1,1-trifluoro-8-methyl-2,10-dioxa-5,8,11-triazatridec-11-en-9-one hydrochloride (41)

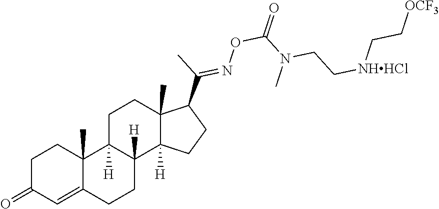

Prepared according to General Procedures A and C. ¹H NMR (400 MHZ, CDCl₃) δ 5.72 (s, 1H), 4.05 (t, J=5.2 Hz, 2H), 3.48-3.38 (m, 2H), 2.99 (s, 3H), 2.94-2.83 (m, 4H), 2.46-2.22 (m, 6H), 2.05-1.98 (m, 1H), 1.93 (s, 3H), 1.92-1.82 (m, 2H), 1.80-1.21 (m, 9H), 1.18 (s, 3H), 1.15-0.91 (m, 3H), 0.72 (s, 3H). ¹³C NMR (101 MHZ, CDCl₃) δ 199.7, 171.3, 165.0, 124.0, 123.0, 120.5, 66.9, 56.8, 55.5, 53.8, 49.2, 47.5, 47.1, 44.2, 38.7, 38.6, 35.8, 35.8, 34.9, 34.1, 32.9, 32.0, 24.3, 23.2, 21.1, 17.5, 17.0, 13.6. HRMS: (APCI+) [M+H]⁺ calc. for C₂₈H₄₃O₄N₃F₃, 542.32002, observed, 542.32119.

### Example 7. Preparation of additional prodrugs of progesterone C20-oxime

Additional prodrugs of progesterone C20-oxime were prepared according to General procedure A described above. A representative synthesis of 36 and 37 is shown below in Scheme 7.

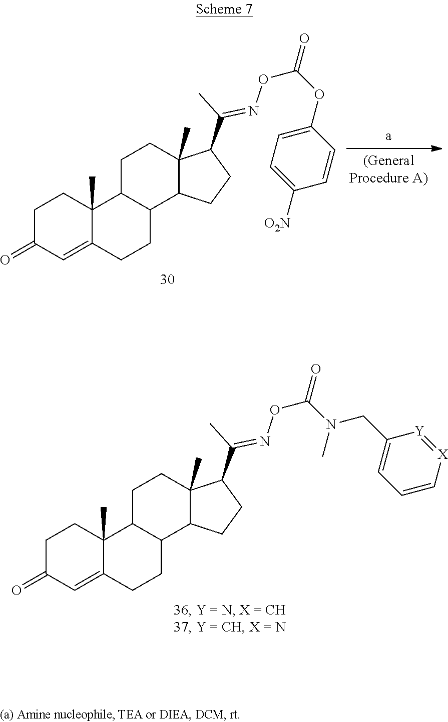

### A. Preparation of (8S,9S,10R,13S,14S,17S)-10,13-dimethyl-17-((E)-1-(((methyl(pyridin-2-ylmethyl)carbamoyl)oxy)imino)ethyl)-1,2,6,7,8,9,10,11,12,13,14,15,16,17-tetradecahydro-3H-cyclopenta[a]phenanthren-3-one (36)

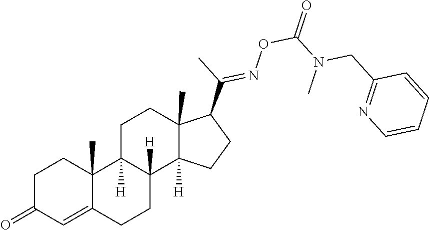

Prepared according to General Procedure A using N-methyl-1-(pyridin-2-yl) methanamine as the amine nucleophile with the following exception: diisopropylethylamine was used instead of triethylamine. The product was in the form of a white solid (215 mg, 0.450 mmol, 74% yield). ¹H NMR (600 MHZ, CDCl₃) δ 8.54 (s, 1H), 7.67 (s, 1H), 7.41-7.22 (m, 1H), 7.18 (dd, J=7.5, 4.9 Hz, 1H), 5.72 (s, 1H), 4.64 (s, 2H), 3.07 (s, 3H), 3.01* (s, 3H), 2.48-2.19 (m, 6H), 2.08-1.82 (m, 4H), 1.82-1.63 (m, 4H), 1.64-1.50 (m, 2H), 1.47-1.21 (m, 4H), 1.17 (s, 3H), 1.15-1.09 (m, 1H), 1.09-1.00 (m, 1H), 1.00-0.92 (m, 1H), 0.74 (s, 3H), 0.69* (s, 3H). ¹³C NMR (151 MHz, CDCl₃) δ 199.6, 171.2, 165.0, 157.6, 155.4, 149.6, 137.0, 124.0, 122.5, 120.5, 56.9, 55.5, 55.0, 54.7, 53.9, 44.2, 38.7, 38.6, 35.9, 34.5, 34.1, 32.9, 32.0, 24.3, 23.2, 21.1, 17.5, 17.0, 13.6. LC-MS (ESI+) [M+Na]⁺ calc. for C₂₉H₃₉N₃NaO₃⁺, 500.3, observed, 500.2.

### B. Preparation of (8S,9S,10R,13S,14S,17S)-10,13-dimethyl-17-((E)-1-(((methyl(pyridin-3-ylmethyl)carbamoyl)oxy)imino)ethyl)-1,2,6,7,8,9,10,11,12,13,14,15,16,17-tetradecahydro-3H-cyclopenta[a]phenanthren-3-one (37)

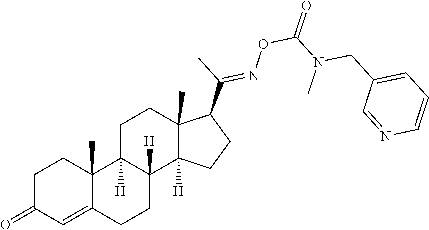

Prepared according to General Procedure A using N-methyl-1-(pyridin-3-yl) methanamine as the amine nucleophile except that diisopropylethylamine was used instead of triethylamine. The product was in the form of a white solid (221 mg, 0.463 mmol, 76% yield). ¹H NMR (600 MHZ, CDCl₃) δ 8.56 (s, 2H), 7.78-7.52 (m, 1H), 7.35-7.19 (m, 1H), 5.73 (s, 1H), 4.54 (s, 2H), 3.03-2.84 (m, 3H), 2.48-2.19 (m, 6H), 2.05-2.00 (m, 1H), 1.99-1.68 (m, 8H), 1.64-1.53 (m, 2H), 1.44 (qd, J=13.0, 3.8 Hz, 1H), 1.39-1.23 (m, 2H), 1.19 (s, 3H), 1.18-1.13 (m, 1H), 1.11-1.02 (m, 1H), 0.98 (td, J=11.5, 4.0 Hz, 1H), 0.74 (s, 3H). ¹³C NMR (151 MHZ, CDCl₃) δ 199.5, 171.2, 165.2, 155.4, 149.6, 149.2, 136.2, 132.9, 124.0, 123.8, 56.9, 55.5, 53.8, 50.5, 44.2, 38.7, 38.7, 38.6, 35.8, 35.8, 34.1, 32.9, 32.0, 24.3, 23.2, 21.1, 17.5, 17.0, 13.6. LC-MS (ESI+) [M+H]⁺ calc. for C₂₉H₄₀N₃O₃, 478.3, observed, 478.3.

### C. Preparation of (8S,9S,10R,13S,14S,17S)-17-((E)-1-((((3-(hydroxymethyl)pyridin-2-yl)(methyl)carbamoyl)oxy)imino)ethyl)-10,13-dimethyl-1,2,6,7,8,9,10,11,12,13,14,15,16,17-tetradecahydro-3H-cyclopenta[a]phenanthren-3-one (38)

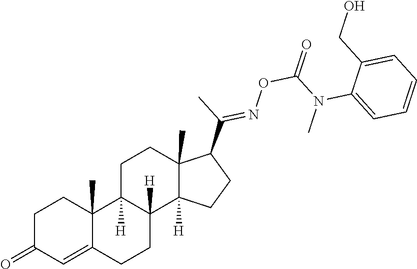

Prepared according to General Procedure A using (2-(methylamino)pyridin-3-yl) methanol as the amine nucleophile. The product was in the form of a white solid (124 mg, 0.2512 mmol, 41.138% yield). ¹H NMR (600 MHZ, CDCl₃) δ 8.18 (dd, J=5.1, 1.8 Hz, 1H), 7.43 (dd, J=7.2, 1.9 Hz, 1H), 6.57 (dd, J=7.2, 5.0 Hz, 1H), 5.73 (d, J=1.7 Hz, 1H), 5.14 (s, 2H), 5.09 (s, 1H), 3.03 (s, 3H), 3.03* (s, 3H), 2.48-2.19 (m, 6H), 2.06-1.99 (m, 1H), 1.96 (s, 3H), 1.95-1.83 (m, 1H), 1.80-1.67 (m, 3H), 1.62-1.53 (m, 3H), 1.47-1.40 (m, 1H), 1.38-1.22 (m, 2H), 1.18 (s, 3H), 1.17-1.13 (m, 1H), 1.11-1.00 (m, 1H), 1.01-0.92 (m, 1H), 0.71 (s, 3H). ¹³C NMR (151 MHz, CDCl₃) δ 199.6, 171.1, 166.8, 158.0, 154.3, 149.1, 139.3, 124.1, 114.0, 112.2, 67.8, 56.8, 55.6, 53.9, 44.3, 38.7, 38.6, 35.9, 34.1, 32.9, 32.0, 29.8, 28.9, 24.2, 23.2, 21.1, 17.5, 17.1, 13.6. LC-MS (ESI+) [M+H]⁺ calc. for C₂₉H₄₀N₃O₄⁺, 494.3, observed, 494.3.

### Example 8. Preparation of additional amine linkers for prodrugs of progesterone C20-oxime

Additional amine linkers were prepared according to General procedure D described below.

General procedure D: To an oven-dried 2-neck round bottom flask, equipped with stirrer bar and molecular sieves (3 Å), was added tert-butyl methyl(2-oxoethyl)carbamate (5) in anhydrous MeOH under an argon atmosphere. The amine nucleophile (2 equiv.) was then added, and the reaction mixture stirred overnight at room temperature. For amine hydrochloride reagents TEA (2 equiv.) was added additionally. The reaction mixture was then treated with palladium on carbon (15 mol %) and stirred under hydrogen atmosphere for 4 hours. Following filtration, the solvent was concentrated in vacuo and purified using flash chromatography.

### A. Preparation of tert-butyl(2-(isopropylamino)ethyl)(methyl)carbamate (39)

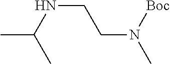

Prepared according to General Procedure D using commercially available propan-2-amine. Rf=0.45 (15% MeOH/DCM). The product was in the form of an oil (507 mg, 2.344 mmol, 52% yield).

### B. Preparation of tert-butyl(2-(cyclopropylamino)ethyl)(methyl)carbamate (40)

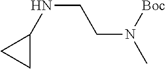

Prepared according to General Procedure D using commercially available cyclopropanamine. Rf=0.57 (15% MeOH/DCM). The product was in the form of an oil (2224 mg, 10.378 mmol, 81% yield).

### C. Preparation of tert-butyl(2-(cyclobutylamino)ethyl)(methyl)carbamate (41)

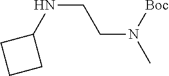 [SMILES: CN(CCNC1CCC1)C(=O)OC(C)(C)C]

Prepared according to General Procedure D using commercially available cyclobutanamine. Rf=0.52 (15% MeOH/DCM). The product was in the form of an oil (870 mg, 3.81 mmol, 66% yield).

### D. Preparation of tert-butyl(2-(cyclopentylamino)ethyl)(methyl)carbamate (42)

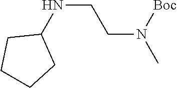 [SMILES: CN(CCNC1CCCC1)C(=O)OC(C)(C)C]

Prepared according to General Procedure D using commercially available cyclopentanamine. Rf=0.50 (15% MeOH/DCM). The product was in the form of an oil (966 mg, 3.984 mmol, 69% yield).

### E. Preparation of tert-butyl(2-(isobutylamino)ethyl)(methyl)carbamate (43)

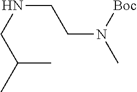 [SMILES: CC(C)CNCCN(C)C(=O)OC(C)(C)C]

Prepared according to General Procedure D using commercially available 2-methylpropan-1-amine. Rf=0.56 (15% MeOH/DCM). The product was in the form of an oil (958 mg, 4.157 mmol, 72% yield).

### F. Preparation of tert-butyl(2-((2-(dimethylamino)ethyl)amino)ethyl)(methyl)carbamate (44)

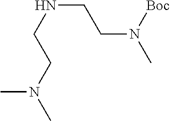 [SMILES: CN(C)CCNCCN(C)C(=O)OC(C)(C)C]

Prepared according to General Procedure D using commercially available N₁,N₁-dimethylethane-1,2-diamine. Rf=0.21 (15% MeOH/DCM). The product was in the form of an oil (978 mg, 3.984 mmol, 69% yield).

### G. Preparation of tert-butyl(2-((tert-butoxycarbonyl)(methyl)amino)ethyl) glycinate (45)

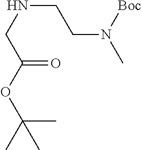 [SMILES: CN(CCNCC(=O)OC(C)(C)C)C(=O)OC(C)(C)C]

Prepared according to General Procedure D using commercially available tert-butyl glycinate. Rf=0.58 (10% MeOH/DCM). The product was in the form of an oil (1015 mg, 3.522 mmol, 61% yield).

### H. Preparation of tert-butyl 3-((2-((tert-butoxycarbonyl)(methyl)amino)ethyl)amino) propanoate (46)

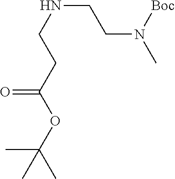 [SMILES: CN(CCNCCC(=O)OC(C)(C)C)C(=O)OC(C)(C)C]

Prepared according to General Procedure D using commercially available tert-butyl 3-aminopropanoate hydrochloride.

### Example 9. Preparation of di-tert-butyl(azanediylbis(ethane-2,1-diyl))bis(methylcarbamate)(47)

di-tert-Butyl (azanediylbis(ethane-2,1-diyl))bis(methylcarbamate)(47) was prepared according to Scheme 8.

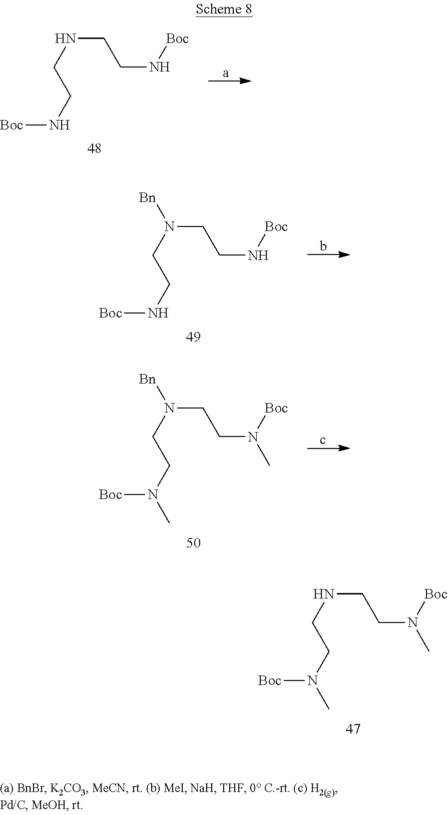

### A. Preparation of di-tert-butyl((benzylazanediyl)bis(ethane-2,1-diyl))dicarbamate (49)

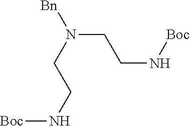 [SMILES: CC(C)(C)OC(=O)NCCN(CCNC(=O)OC(C)(C)C)Cc1ccccc1]

To an oven-dried 2-neck round bottom flask was added commercially available di-tert-butyl(azanediylbis(ethane-2,1-diyl))dicarbamate (48)(1000 mg, 3.296 mmol) and anhydrous MeCN (30 mL). The solution was then treated with K₂CO₃ at room temperature and stirred for 15 minutes. Benzyl bromide was then added dropwise and the reaction mixture was stirred overnight. Following filtration, the solvent was removed in vacuo and flash chromatography (20-50% ethyl acetate/hexane) afforded the product di-tert-butyl((benzylazanediyl)bis(ethane-2,1-diyl))dicarbamate (49)(1260 mg, 3.202 mmol, 97%) as a clear viscous oil.

### B. Preparation of di-tert-butyl((benzylazanediyl)bis(ethane-2,1-diyl))bis(methylcarbamate)(50)

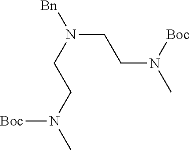 [SMILES: CN(CCN(CCN(C)C(=O)OC(C)(C)C)Cc1ccccc1)C(=O)OC(C)(C)C]

An oven-dried 100 mL 2-neck round bottom flask was charged with a stirrer bar and di-tert-butyl((benzylazanediyl)bis(ethane-2,1-diyl))dicarbamate (49)(1.24 g, 3.15 mmol) under argon. Anhydrous THF (30 mL) was then added, and the mixture was cooled to 0° C. in a brine ice bath. Sodium hydride (502 mg, 12.604 mmol) was then added to the mixture. After stirring for 30 minutes, methyl iodide (1.18 mL, 18.906 mmol) was added dropwise to mixture at 0° C., and the resulting mixture was allowed to warm to room temperature and stirred overnight (18 h). Afterward, the reaction mixture was quenched by adding a few drops of DI water and then pouring the mixture into saturated ammonium chloride solution (150 mL). The organic layer was then washed with brine and dried over anhydrous sodium sulfate. The solvent was removed in vacuo and the product was purified by flash chromatography (20-50% ethyl acetate/hexanes) to afford di-tert-butyl ((benzylazanediyl)bis(ethane-2,1-diyl))bis(methylcarbamate)(50)(1.169 g, 2.772 mmol, 88% yield) as an oil.

### C. Preparation of di-tert-butyl(azanediylbis(ethane-2,1-diyl))bis(methylcarbamate)(47)

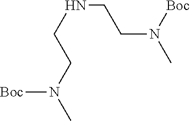 [SMILES: CN(CCNCCN(C)C(=O)OC(C)(C)C)C(=O)OC(C)(C)C]

An oven-dried 100 mL round bottom flask was charged with a stirrer bar, di-tert-butyl ((benzylazanediyl)bis(ethane-2,1-diyl))bis(methylcarbamate)(50)(450.00 mg, 1.140 mmol), palladium on carbon (30 mg, 0.285 mmol), and anhydrous methanol (6 mL) under argon. A T-joint bearing a hydrogen balloon and Schlenk line connection was affixed to the flask. The reaction flask was then briefly evacuated and filled with hydrogen. This process of hydrogen filling was repeated 3 times. The mixture was then allowed to stir at room temperature under hydrogen for 5 h. Afterward, the mixture was filtered over celite, with the celite further washed with methanol. The combined eluants were then concentrated in vacuo to afford a colorless oil that was immediately carried forward to the next reaction.

### Example 10. Preparation of tert-butyl(S)-2-(2-(methylamino)ethyl)pyrrolidine-1-carboxylate (51)

tert-Butyl(S)-2-(2-(methylamino)ethyl)pyrrolidine-1-carboxylate (51) was prepared according to Scheme 9.

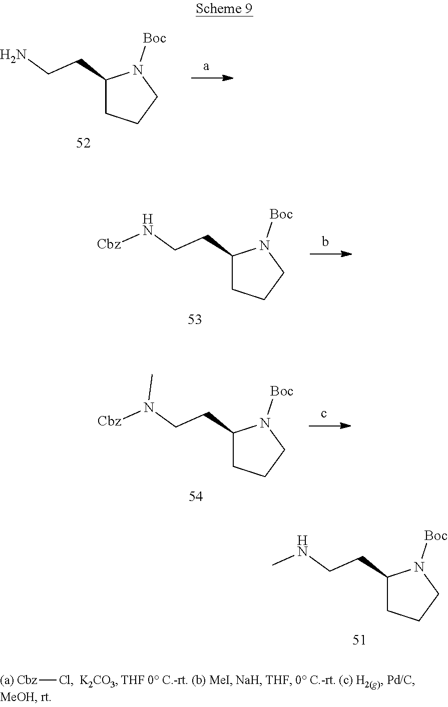 [SMILES: CC(C)(C)OC(=O)N1CCC[C@H]1CCN ; CC(C)(C)OC(=O)N1CCC[C@H]1CCNC(=O)OCc1ccccc1 ; CN(CC[C@@H]1CCCN1C(=O)OC(C)(C)C)C(=O)OCc1ccccc1 ; CNCC[C@@H]1CCCN1C(=O)OC(C)(C)C ; O=C(Cl)OCc1ccccc1]

### A. Preparation of tert-butyl(S)-2-(2-(((benzyloxy)carbonyl)amino)ethyl)pyrrolidine-1-carboxylate (53)

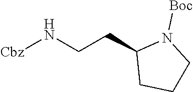 [SMILES: CC(C)(C)OC(=O)N1CCC[C@H]1CCNC(=O)OCc1ccccc1]

An oven-dried 100 mL Schlenk tube was charged with a stirrer bar and tert-butyl(S)-2-(2-aminoethyl)pyrrolidine-1-carboxylate (52)(500.00 mg, 2.33 mmol) and placed under argon. Anhydrous THF (8 mL) was then added followed by potassium carbonate (645 g, 4.67 mmol). The mixture was then cooled to 0° C. in a brine ice bath. Benzyl chloroformate (0.4 mL, 2.80 mmol) was then added dropwise, and the mixture was allowed to warm to room temperature and stirred overnight. Afterwards, the reaction was quenched with 10 mL water and extracted with 30 mL ethyl acetate. The organic layer was separated and washed with saturated ammonium chloride solution and brine. The organic layer was then dried over anhydrous sodium sulfate and concentrated in vacuo to afford an oil. Flash chromatography (0-50% ethyl acetate/hexanes) afforded tert-butyl(S)-2-(2-(((benzyloxy)carbonyl)amino)ethyl)pyrrolidine-1-carboxylate (53) (799 mg, 2.293 mmol, 98% yield) as a colorless oil.

### B. Preparation of tert-butyl(S)-2-(2-(((benzyloxy)carbonyl)(methyl)amino)ethyl)pyrrolidine-1-carboxylate (54)

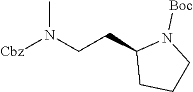 [SMILES: CN(CC[C@@H]1CCCN1C(=O)OC(C)(C)C)C(=O)OCc1ccccc1]

An oven-dried 100 mL Schlenk tube was charged with a stirrer bar and tert-butyl(S)-2-(2-(((benzyloxy)carbonyl)amino)ethyl)pyrrolidine-1-carboxylate (53)(775 mg, 2.221 mmol) under argon. Anhydrous THF (10 mL) was then added, and the mixture was cooled to 0° C. in a brine ice bath. Sodium hydride (177 mg, 4.442 mmol) was then added to the mixture. After stirring for 30 minutes, methyl iodide (560 μL, 8.884 mmol) was added dropwise to mixture at 0° C., and the resulting mixture was allowed to warm to room temperature and stirred overnight (18 h). Afterward, the reaction mixture was quenched by adding a few drops of DI water and then pouring the mixture into saturated ammonium chloride solution (40 mL). The organic layer was then washed with brine and dried over anhydrous sodium sulfate. Concentration in vacuo afforded an orange oil that was subjected to flash chromatography (0-50% ethyl acetate in hexanes) to afford tert-butyl(S)-2-(2-(((benzyloxy)carbonyl)(methyl)amino)ethyl)pyrrolidine-1-carboxylate (54)(685 g, 1.890 mmol, 85% yield) as a yellow oil.

### C. Preparation of tert-butyl(S)-2-(2-(methylamino)ethyl)pyrrolidine-1-carboxylate (51)

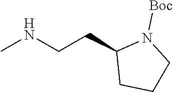 [SMILES: CNCC[C@@H]1CCCN1C(=O)OC(C)(C)C]

An oven-dried 100 mL round bottom flask was charged with a stirrer bar, tert-butyl(S)-2-(2-(((benzyloxy)carbonyl)(methyl)amino)ethyl)pyrrolidine-1-carboxylate (54)(660.90 mg, 1.823 mmol), palladium on carbon (39 mg, 0.364 mmol), and anhydrous methanol (10 mL) under argon. A T-joint bearing a hydrogen balloon and Schlenk line connection was affixed to the flask. The reaction flask was then briefly evacuated and filled with hydrogen. This process of hydrogen filling was repeated 3 times. The mixture was then allowed to stir at room temperature under hydrogen for 2 h. Afterward, the mixture was filtered over celite, with the celite further washed with methanol. The combined eluants were then concentrated in vacuo to afford a colorless oil that was immediately carried forward to the next reaction.

### Example 11. Preparation of Additional Prodrugs of Progesterone C20-Oxime

Prodrugs of progesterone C20-oxime were prepared according to the General Procedures A, B and C previously described.

### AA. Preparation of tert-butyl(2-((((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)(isopropyl)amino)ethyl)(methyl)carbamate (52a)

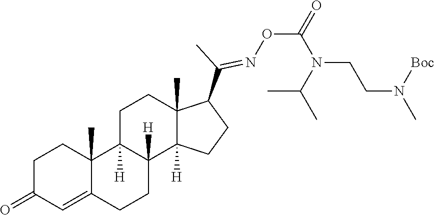

Prepared according to General Procedure B using amine nucleophile 39. Rf=0.32 (20% ethyl acetate/DCM). The product was in the form of a white solid (630 mg, 1.102 mmol, 70% yield).

### AB. Preparation of tert-butyl(2-(cyclopropyl(((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)amino)ethyl)(methyl)carbamate (52b)

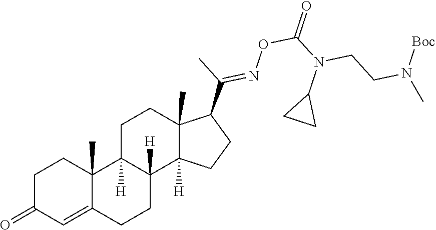

Prepared according to General Procedure B using amine nucleophile 40. Rf=0.25 (20% ethyl acetate/DCM). The product was in the form of a white solid (673 mg, 1.181 mmol, 83% yield).

### AC. Preparation of tert-butyl(2-(cyclobutyl(((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)amino)ethyl)(methyl)carbamate (52c)

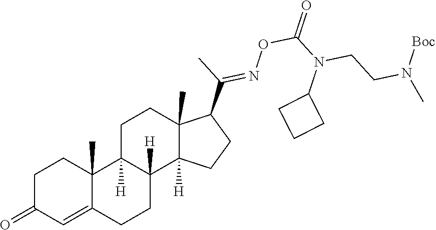

Prepared according to General Procedure B using amine nucleophile 41. Rf=0.31 (20% ethyl acetate/DCM). The product was in the form of a white solid (473 mg, 0.810 mmol, 80% yield).

### AD. Preparation of tert-butyl(2-(cyclopentyl(((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)amino)ethyl)(methyl)carbamate (52d)

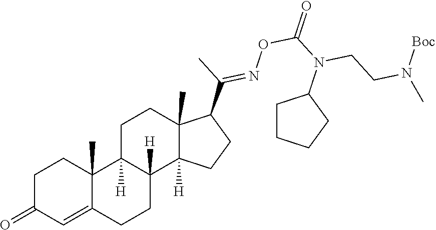

Prepared according to General Procedure B using amine nucleophile 42. Rf=0.30 (10% ethyl acetate/DCM). The product was in the form of a white solid (520 mg, 0.870 mmol, 61% yield).

### AE. Preparation of tert-butyl(2-((((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)(isobutyl)amino)ethyl)(methyl)carbamate (52e)

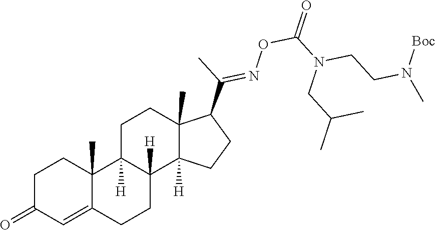

Prepared according to General Procedure B using amine nucleophile 43. Rf=0.45 (20% ethyl acetate/DCM). The product was in the form of a white solid (330 mg, 0.564 mmol, 70% yield).

### AF. Preparation of tert-butyl(2-((((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)(2-(dimethylamino)ethyl)amino)ethyl)(methyl)carbamate (52f)

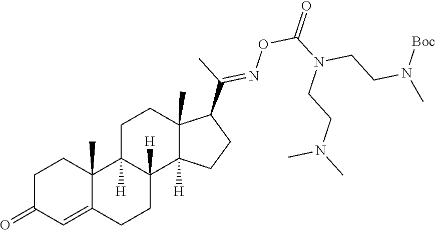

Prepared according to General Procedure B using amine nucleophile 44. Rf=0.28 (40% ethyl acetate/DCM). The product was in the form of a white solid (365 mg, 0.608 mmol, 77% yield).

### AG. Preparation of tert-butyl N-(2-((tert-butoxycarbonyl)(methyl)amino)ethyl)-N-(((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)glycinate (52g)

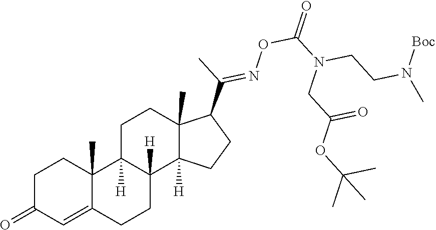

Prepared according to General Procedure B using amine nucleophile 45. Rf=0.51 (20% ethyl acetate/DCM). The product was in the form of a white solid (531 mg, 0.825 mmol, 82% yield).

### AH. Preparation of tert-butyl 3-((2-((tert-butoxycarbonyl)(methyl)amino)ethyl)(((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)amino) propanoate (52h)

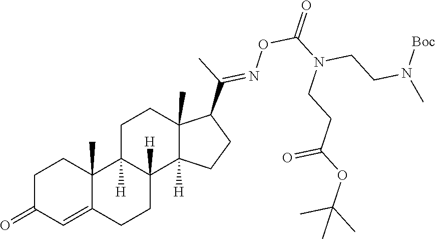

Prepared according to General Procedure B using amine nucleophile 46. Rf=0.48 (20% ethyl acetate/DCM). The product was in the form of a white solid (283 mg, 0.430 mmol, 71% yield).

### AI. Preparation of di-tert-butyl(((((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl) azanediyl)bis(ethane-2,1-diyl))bis(methylcarbamate)(52i)

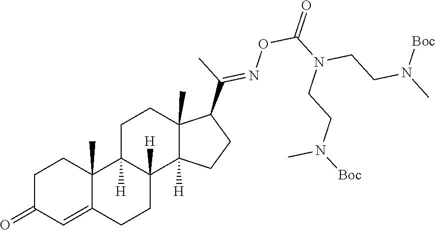

Prepared according to General Procedure B using amine nucleophile 47. Rf=0.32 (30% ethyl acetate/DCM). The product was in the form of a white solid (202 mg, 0.294 mmol, 73% yield).

### AJ. Preparation of tert-butyl(2-(cyclopropyl(((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)amino)ethyl)(methyl)carbamate (52j)

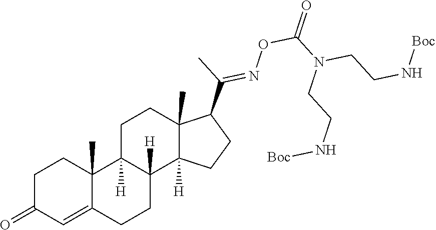

Prepared according to General Procedure B using amine nucleophile 48. Rf=0.30 (20% ethyl acetate/DCM). The product was in the form of a white solid (405 mg, 0.615 mmol, 76% yield).

### AK. Preparation of tert-butyl(S)-2-(2-((((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)(methyl)amino)ethyl)pyrrolidine-1-carboxylate (52k)

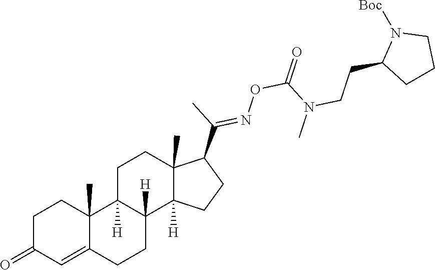

Prepared according to General Procedure A using amine nucleophile 51. The product was in the form of an off-white solid (887 mg, 1.519 mmol, 92% yield).

### AL. Preparation of (E)-9-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)-5-isopropyl-7-oxa-2,5,8-triazadec-8-en-6-one 2,2,2-trifluoroacetate (53a)

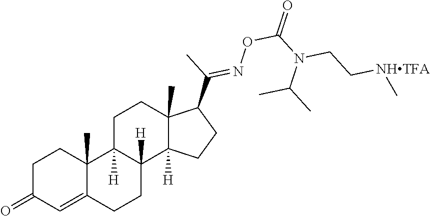

Prepared according to General Procedure C to afford the TFA salt. The product was in the form of a white solid (193 mg, 0.329 mmol, 94% yield). ¹H NMR (600 MHZ, DMSO) § 8.52 (br s, 2H), 5.63 (s, 1H), 4.12 (hept, J=6.8 Hz, 1H), 3.42 (t, J=7.0 Hz, 2H), 3.06-3.01 (m, 2H), 2.60 (s, 3H), 2.45-2.35 (m, 3H), 2.28-2.22 (m, 1H), 2.19-2.11 (m, 2H), 2.01-1.94 (m, 1H), 1.93 (s, 3H), 1.90-1.85 (m, 1H), 1.83-1.77 (m, 1H), 1.70-1.50 (m, 5H), 1.42-1.28 (m, 2H), 1.25-1.17 (m, 2H), 1.15 (s, 3H), 1.15 (s, 6H), 1.03-0.90 (m, 2H), 0.65 (s, 3H); ¹³C NMR (151 MHZ, DMSO) δ 198.0, 170.8, 164.8, 154.3, 123.2, 55.9, 54.7, 53.1, 48.2, 47.9, 43.4, 38.2, 37.7, 35.1, 35.1, 33.6, 33.0, 31.9, 31.6, 23.7, 22.6, 20.6, 20.3, 17.0, 16.9, 13.1. One 13C signal not observed. HRMS (APCI+) [M+H]⁺ calc. for C₂₈H₄₆N₃O₃, 472.35337, observed, 472.35412.

### AM. Preparation of (E)-5-cyclopropyl-9-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)-7-oxa-2,5,8-triazadec-8-en-6-one 2,2,2-trifluoroacetate (53b)

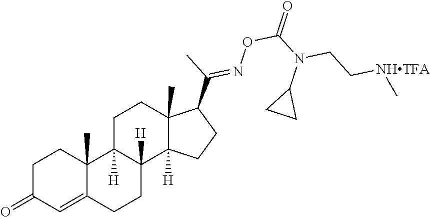

Prepared according to General Procedure C to afford the TFA salt. The product was in the form of a white solid (290 mg, 0.514 mmol, 94% yield). ¹H NMR (600 MHZ, DMSO) δ 8.56 (br s, 2H), 5.64 (s, 1H), 3.51 (t, J=6.4 Hz, 2H), 3.11-3.06 (m, 2H), 2.73-2.67 (m, 1H), 2.59 (s, 3H), 2.45-2.34 (m, 3H), 2.28-2.22 (m, 1H), 2.20-2.12 (m, 2H), 2.01-1.97 (m, 1H), 1.96 (s, 3H), 1.91-1.85 (m, 1H), 1.84-1.77 (m, 1H), 1.71-1.50 (m, 5H), 1.43-1.29 (m, 2H), 1.26-1.16 (m, 2H), 1.15 (s, 3H), 1.04-0.90 (m, 2H), 0.79-0.74 (m, 2H), 0.73-0.69 (m, 2H), 0.65 (s, 3H); ¹³C NMR (151 MHZ, DMSO) δ 198.0, 170.8, 164.7, 155.2, 123.2, 55.9, 54.7, 53.1, 46.9, 43.9, 43.4, 38.2, 37.8, 35.1, 35.1, 33.6, 32.9, 31.9, 31.6, 28.8, 23.7, 22.6, 20.6, 17.0, 16.9, 13.1, 7.7. One 13C signal not observed. HRMS (APCI+) [M+H]⁺ calc. for C₂₈H₄₄N₃O₃, 470.33772, observed, 470.33841.

### AN. Preparation of (E)-5-cyclobutyl-9-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)-7-oxa-2,5,8-triazadec-8-en-6-one 2,2,2-trifluoroacetate (53c)

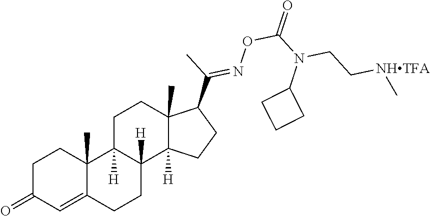

Prepared according to General Procedure C to afford the TFA salt. The product was in the form of a white solid (450 mg, 0.753 mmol, 96% yield). ¹H NMR (600 MHZ, DMSO) δ 8.59 (br s, 2H), 5.63 (s, 1H), 4.30-4.24 (m, 1H), 3.54 (t, J=6.8 Hz, 2H), 3.01-2.98 (m, 2H), 2.60 (s, 3H), 2.45-2.34 (m, 3H), 2.28-2.21 (m, 1H), 2.19-2.07 (m, 6H), 2.01-1.95 (m, 1H), 1.94 (s, 3H), 1.90-1.84 (m, 1H), 1.84-1.77 (m, 1H), 1.70-1.50 (m, 7H), 1.42-1.28 (m, 2H), 1.26-1.17 (m, 2H), 1.15 (s, 3H), 1.03-0.90 (m, 2H), 0.65 (s, 3H); ¹³C NMR (151 MHZ, DMSO) & 198.0, 170.8, 164.9, 154.1, 123.2, 55.9, 54.7, 53.1, 50.6, 47.8, 43.4, 38.2, 37.7, 35.1, 35.1, 33.6, 33.0, 31.9, 31.6, 28.5, 23.7, 22.5, 20.6, 17.0, 16.9, 14.1, 13.1. Two 13C signals not observed. HRMS (APCI+) [M+H]⁺ calc. for C₂₉H₄₆N₃O₃, 484.35337, observed, 484.35406.

### AO. Preparation of (E)-5-cyclopentyl-9-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)-7-oxa-2,5,8-triazadec-8-en-6-one 2,2,2-trifluoroacetate (53d)

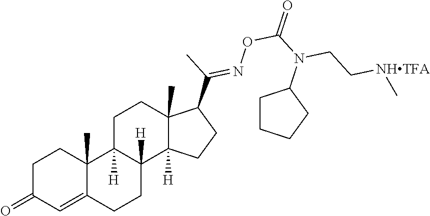

Prepared according to General Procedure C to afford the TFA salt. The product was in the form of a white solid (298 mg, 0.487 mmol, 95% yield). ¹H NMR (600 MHZ, DMSO) δ 8.62 (br s, 2H), 5.63 (s, 1H), 4.16 (p, J=8.5 Hz, 1H), 3.42 (t, J=7.1 Hz, 2H), 3.06-3.00 (m, 2H), 2.60 (s, 3H), 2.45-2.33 (m, 3H), 2.28-2.22 (m, 1H), 2.21-2.11 (m, 2H), 2.00-1.94 (m, 1H), 1.93 (s, 3H), 1.90-1.85 (m, 1H), 1.85-1.77 (m, 3H), 1.71-1.47 (m, 11H), 1.42-1.28 (m, 2H), 1.26-1.16 (m, 2H), 1.15 (s, 3H), 1.03-0.90 (m, 2H), 0.65 (s, 3H); ¹³C NMR (151 MHZ, DMSO) & 198.0, 170.8, 164.9, 154.3, 123.2, 57.8, 55.9, 54.7, 53.1, 47.8, 43.4, 38.2, 37.7, 35.1, 35.1, 33.6, 33.0, 31.9, 31.6, 28.9, 23.7, 23.1, 22.6, 20.6, 17.0, 16.9, 13.1. One 13C signal not observed. HRMS (APCI+) [M+H]⁺ calc. for C₃₀H₄₈N₃O₃, 498.36902, observed, 498.36952.

### AP. Preparation of (E)-9-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)-5-isobutyl-7-oxa-2,5,8-triazadec-8-en-6-one 2,2,2-trifluoroacetate (53e)

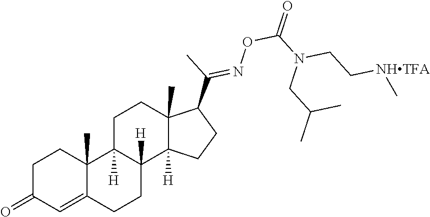

Prepared according to General Procedure C to afford the TFA salt. The product was in the form of a white solid (285 mg, 0.475 mmol, 93% yield). ¹H NMR (600 MHZ, DMSO) δ 8.68 (br s, 1H), 8.52 (br s, 1H), 5.64 (s, 1H), 3.55-3.45 (m, 2H), 3.12-3.02 (m, 4H), 2.59 (s, 3H), 2.45-2.33 (m, 3H), 2.28-2.22 (m, 1H), 2.21-2.11 (m, 2H), 2.00-1.84 (m, 6H), 1.84-1.77 (m, 1H), 1.70-1.50 (m, 5H), 1.43-1.28 (m, 2H), 1.26-1.16 (m, 2H), 1.15 (s, 3H), 1.03-0.90 (m, 2H), 0.85 (d, J=6.7 Hz, 6H), 0.64 (s, 3H); ¹³C NMR (151 MHZ, DMSO) δ 198.0, 170.8, 164.3, 154.5, 123.2, 55.9, 54.7, 54.1, 53.1, 46.5, 43.9, 43.4, 38.2, 37.7, 35.1, 35.1, 33.6, 32.9, 31.9, 31.6, 27.0, 23.7, 22.5, 20.5, 19.8, 16.9, 13.1. One 13C signal not observed. HRMS (APCI+) [M+H]⁺ calc. for C₂₉H₄₈N₃O₃, 486.36902, observed, 486.36976.

### AQ. Preparation of (E)-9-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)-2-methyl-5-(2-(methylamino)ethyl)-7-oxa-2,5,8-triazadec-8-en-6-one 2,2,2-trifluoroacetate (53f)

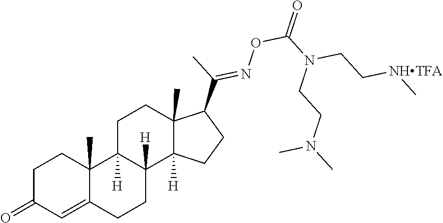

Prepared according to General Procedure C to afford the TFA salt. The product was in the form of a white solid (325 mg, 0.529 mmol, 89% yield). ¹H NMR (600 MHZ, DMSO) δ 9.10-8.50 (m, 2H), 5.64 (s, 1H), 3.64-3.52 (m, 4H), 3.25-3.16 (m, 2H), 3.12 (t, J=6.3 Hz, 2H), 2.86-2.70 (m, 6H), 2.60 (s, 3H), 2.45-2.36 (m, 3H), 2.28-2.22 (m, 1H), 2.21-2.11 (m, 2H), 2.02-1.97 (m, 1H), 1.96 (s, 3H), 1.89-1.84 (m, 1H), 1.84-1.77 (m, 1H), 1.71-1.50 (m, 6H), 1.43-1.30 (m, 2H), 1.26-1.17 (m, 1H), 1.15 (s, 3H), 1.03-0.90 (m, 2H), 0.65 (s, 3H); ¹³C NMR (151 MHz, DMSO) δ 198.0, 170.8, 165.3, 154.0, 123.2, 55.9, 54.7, 54.3, 53.1, 46.8, 43.5, 42.8, 42.3, 38.2, 37.7, 35.1, 35.1, 33.6, 32.8, 31.9, 31.6, 23.7, 22.5, 20.5, 17.0, 16.9, 13.1. One 13C signal not observed. HRMS (APCI+) [M+H]⁺ calc. for C₂₉H₄₉N₄O₃, 501.37992, observed, 501.38053.

### AR. Preparation of N-(((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)-N-(2-(methylamino)ethyl)glycine 2,2,2-trifluoroacetate (53g)

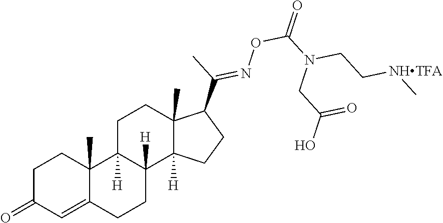

Prepared according to General Procedure C to afford the TFA salt. The product was in the form of a white solid (126 mg, 0.209 mmol, 56% yield). ¹H NMR (600 MHZ, CDCl₃) δ 10.59 (br s, 2H), 5.70 (s, 1H), 3.92 (d, J=17.7 Hz, 1H), 3.85 (d, J=17.7 Hz, 1H), 3.78-3.62 (m, 2H), 3.26-3.14 (m, 2H), 2.68 (s, 3H), 2.45-2.18 (m, 6H), 2.02-1.96 (m, 1H), 1.88 (s, 3H), 1.87-1.80 (m, 2H), 1.79-1.63 (m, 3H), 1.60-1.47 (m, 2H), 1.45-1.34 (m, 1H), 1.33-1.19 (m, 2H), 1.15 (s, 3H), 1.13-1.08 (m, 1H), 1.07-0.97 (m, 1H), 0.97-0.90 (m, 1H), 0.68 (s, 3H); ¹³C NMR (151 MHZ, CDCl₃) δ 199.6, 176.6, 171.1, 165.8, 154.8, 124.0, 56.7, 55.4, 53.8, 53.4, 47.8, 47.4, 44.1, 38.7, 38.5, 35.8, 34.0, 32.8, 32.6, 31.9, 24.2, 23.1, 21.0, 17.5, 17.1, 13.6. One 13C signal not observed. HRMS (APCI+) [M+H]⁺ calc. for C₂₇H₄₁N₃O₅, 488.3119, observed, 488.31253.

### AS. Preparation of 3-((((((E)-1-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)ethylidene)amino)oxy)carbonyl)(2-(methylamino)ethyl)amino) propanoic acid 2,2,2-trifluoroacetate (53h)

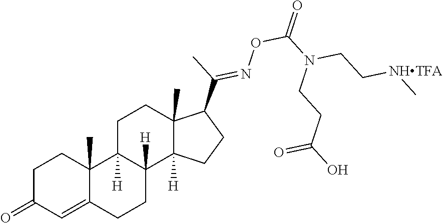

Prepared according to General Procedure C to afford the TFA salt. The product was in the form of a white solid (156 mg, 0.253 mmol, 69% yield). ¹H NMR (600 MHZ, CDCl₃) δ 5.72 (s, 1H), 3.75-3.53 (m, 4H), 3.26-3.17 (m, 2H), 2.67 (s, 3H), 2.62-2.52 (m, 2H), 2.42-2.20 (m, 7H), 2.05-1.98 (m, 1H), 1.95 (s, 3H), 1.94-1.89 (m, 1H), 1.89-1.82 (m, 1H), 1.79-1.66 (m, 4H), 1.63-1.50 (m, 2H), 1.46-1.38 (m, 1H), 1.36-1.21 (m, 3H), 1.18 (s, 3H), 1.17-1.11 (m, 1H), 1.09-1.00 (m, 1H), 0.99-0.94 (m, 1H), 0.71 (s, 3H); ¹³C NMR (151 MHZ, CDCl₃) δ 199.6, 178.3, 171.1, 165.9, 155.9, 124.1, 56.8, 55.5, 53.8, 48.0, 47.3, 46.9, 44.2, 38.7, 38.6, 37.0, 35.8, 34.1, 33.1, 32.9, 32.0, 23.2, 21.1, 17.5, 17.4, 17.2, 13.6. One 13C signal not observed. HRMS (APCI+) [M+H]⁺ calc. for C₂₈H₄₄N₃O₅, 502.32755, observed, 502.32826.

### AT. Preparation of (E)-9-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)-5-(2-(methylamino)ethyl)-7-oxa-2,5,8-triazadec-8-en-6-one dihydrochloride (53i)

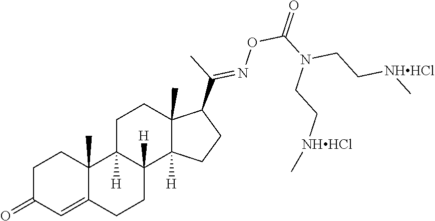

Prepared according to General Procedure C. The product was in the form of a white solid (152 mg, 0.272 mmol, 78% yield). ¹H NMR (600 MHz, DMSO) δ 9.50-8.88 (m, 4H), 5.63 (s, 1H), 3.68-3.56 (m, 4H), 3.14-3.05 (m, 4H), 2.54 (s, 6H), 2.44-2.35 (m, 3H), 2.28-2.21 (m, 1H), 2.19-2.10 (m, 2H), 2.00 (s, 3H), 1.99-1.94 (m, 1H), 1.89-1.84 (m, 1H), 1.83-1.76 (m, 1H), 1.68-1.50 (m, 5H), 1.40-1.29 (m, 2H), 1.26-1.15 (m, 2H), 1.14 (s, 3H), 1.02-0.90 (m, 2H), 0.65 (s, 3H); ¹³C NMR (151 MHZ, DMSO) δ 198.1, 170.9, 165.5, 154.1, 123.2, 56.0, 54.7, 53.1, 46.4, 43.7, 43.5, 38.2, 37.7, 35.1, 35.1, 33.6, 32.6, 32.0, 31.6, 23.7, 22.6, 20.6, 17.4, 16.9, 13.1. HRMS (APCI+) [M+H]⁺ calc. for C₂₈H₄₇N₄O₃, 487.36427, observed, 487.36439.

### AU. Preparation of (8S,9S,10R,13S,14S,17S)-17-((E)-1-(((bis(2-aminoethyl)carbamoyl)oxy)imino)ethyl)-10,13-dimethyl-1,2,6,7,8,9,10,11,12,13,14,15,16,17-tetradecahydro-3H-cyclopenta[a]phenanthren-3-one bis(2,2,2-trifluoroacetate)(53j)

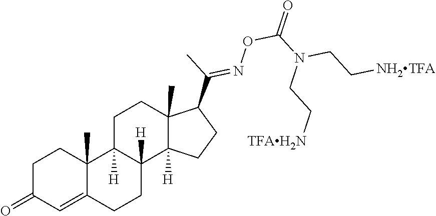

Prepared according to General Procedure C to afford the TFA salt. The product was in the form of a white solid (330 mg, 0.481 mmol, 83% yield). ¹H NMR (600 MHZ, DMSO) δ 8.15-7.83 (m, 6H), 5.63 (s, 1H), 3.53-3.45 (m, 4H), 3.03-2.98 (m, 4H), 2.45-2.36 (m, 3H), 2.28-2.22 (m, 1H), 2.19-2.11 (m, 2H), 2.00-1.97 (m, 1H), 1.95 (s, 3H), 1.89-1.83 (m, 1H), 1.83-1.77 (m, 1H), 1.70-1.50 (m, 5H), 1.40-1.29 (m, 2H), 1.26-1.16 (m, 2H), 1.14 (s, 3H), 1.03-0.90 (m, 2H), 0.65 (s, 3H); ¹³C NMR (151 MHZ, DMSO) δ 198.0, 170.9, 165.3, 154.1, 123.2, 55.9, 54.7, 53.1, 44.9, 43.5, 38.2, 37.8, 37.2, 35.1, 35.1, 33.6, 32.0, 31.6, 23.7, 22.6, 20.6, 17.0, 16.9, 13.1. HRMS (APCI+) [M+H]⁺ calc. for C₂₆H₄₃N₄O₃, 459.33297, observed, 459.33289.

### AV. Preparation of (8S,9S,10R,13S,14S,17S)-10,13-dimethyl-17-((E)-1-(((methyl(2-((S)-pyrrolidin-2-yl)ethyl)carbamoyl)oxy)imino)ethyl)-1,2,6,7,8,9,10,11,12,13,14,15,16,17-tetradecahydro-3H-cyclopenta[a]phenanthren-3-one 2,2,2-trifluoroacetate (53k)

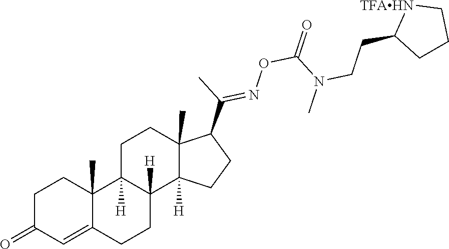

Prepared according to General Procedure C to afford the TFA salt. The product was in the form of a white solid (296 mg, 0.495 mmol, 95% yield). ¹H NMR (600 MHZ, DMSO) δ 9.21-8.90 (m, 1H), 8.63 (br s, 1H), 5.63 (s, 1H), 3.51 (s, 1H), 3.41-3.35 (m, 1H), 3.32 (t, J=7.2 Hz, 2H), 3.22-3.11 (m, 2H), 2.94-2.82 (m, 3H), 2.45-2.32 (m, 3H), 2.28-2.22 (m, 1H), 2.19-2.07 (m, 2H), 2.00-1.94 (m, 3H), 1.91 (s, 3H), 1.90-1.75 (m, 4H), 1.70-1.50 (m, 6H), 1.41-1.28 (m, 2H), 1.26-1.16 (m, 1H), 1.15 (s, 3H), 1.03-0.90 (m, 2H), 0.64 (s, 3H); ¹³C NMR (151 MHz, DMSO) δ 198.0, 170.9, 164.4, 154.0, 123.2, 57.2, 55.9, 54.7, 53.1, 45.9 (2C), 44.4, 43.4, 38.2, 37.8, 35.1, 35.1, 33.6, 32.0, 31.6, 29.5 (2C), 23.7, 23.0, 22.5, 20.6, 16.9, 16.7, 13.1. HRMS (APCI+) [M+H]⁺ calc. for C₂₉H₄₆N₃O₃, 484.35337688.47154, observed, 484.3541.

### AW. Preparation of (E)-2-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)-6-ethyl-4-oxa-3,6,9-triazaundec-2-en-5-one hydrochloride (54a)

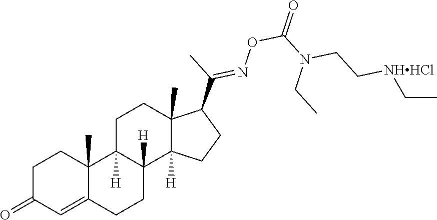

Prepared according to General Procedure B using commercially available N¹,N²-diethylethane-1,2-diamine, followed by treatment with HCl in dioxane (4M). The product was in the form of a white solid (507 mg, 0.737 mmol, 89% yield). ¹H NMR (600 MHZ, DMSO) δ 9.27-8.82 (m, 2H), 5.63 (s, 1H), 3.60-3.48 (m, 2H), 3.32-3.27 (m, 2H), 3.07-3.00 (m, 2H), 2.98-2.91 (m, 2H), 2.45-2.34 (m, 3H), 2.28-2.21 (m, 1H), 2.19-2.11 (m, 2H), 1.99-1.77 (m, 6H), 1.69-1.50 (m, 5H), 1.40-1.28 (m, 2H), 1.20 (t, J=7.2, 1.4 Hz, 3H), 1.14 (s, 3H), 1.13-1.05 (m, 5H), 1.02-0.90 (m, 2H), 0.65 (s, 3H); ¹³C NMR (151 MHZ, DMSO) δ 198.0, 170.9, 164.7, 154.0, 123.2, 55.9, 54.7, 53.1, 44.5, 43.4, 42.1, 38.2, 37.8, 35.1, 35.1, 33.6, 32.0, 31.6, 23.7, 22.5, 20.6, 17.1, 16.9, 16.8, 13.6, 13.1, 12.9, 10.9. HRMS (APCI+) [M+H]⁺ calc. for C₂₈H₄₆N₃O₃, 472.35337, observed, 472.35405.

### AX. Preparation of (E)-2-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)-6-isopropyl-10-methyl-4-oxa-3,6,9-triazaundec-2-en-5-one hydrochloride (54b)

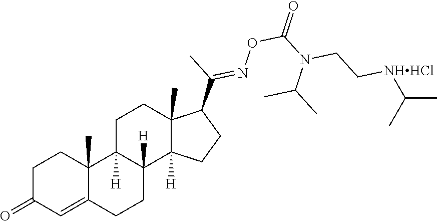

Prepared according to General Procedure B using commercially available N¹,N²-diisopropylethane-1,2-diamine, followed by treatment with HCl in dioxane (4M). The product was in the form of a white solid (187 mg, 0.349 mmol, 76% yield). ¹H NMR (600 MHZ, DMSO) δ 9.23 (br s, 1H), 8.96 (br s, 1H), 5.63 (s, 1H), 4.22-4.09 (m, 1H), 3.55-3.44 (m, 2H), 3.31-3.25 (m, 1H), 3.00-2.94 (m, 2H), 2.45-2.34 (m, 3H), 2.28-2.22 (m, 1H), 2.19-2.11 (m, 2H), 2.00-1.90 (m, 4H), 1.89-1.76 (m, 2H), 1.70-1.50 (m, 5H), 1.40-1.28 (m, 2H), 1.24 (d, J=6.4, 1.6 Hz, 6H), 1.16 (d, J=7.0 Hz, 6H), 1.22-1.18 (m, 2H), 1.15 (s, 3H), 1.03-0.90 (m, 2H), 0.65 (s, 3H); ¹³C NMR (151 MHz, DMSO) δ 198.0, 170.9, 164.7, 153.3, 123.2, 55.9, 54.7, 53.1, 49.5, 47.7, 43.4, 42.9, 38.2, 37.8, 35.1, 35.1, 33.6, 31.9, 31.6, 23.7, 22.5, 20.6, 20.3, 18.5 (2C), 17.1, 16.9 (2C), 13.6, 13.1. HRMS (APCI+) [M+H]⁺ calc. for C₃₀H₅₀N₃O₃, 500.38467, observed, 500.38473.

### AY. Preparation of (E)-6-(tert-butyl)-2-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)-10,10-dimethyl-4-oxa-3,6,9-triazaundec-2-en-5-one hydrochloride (54c)

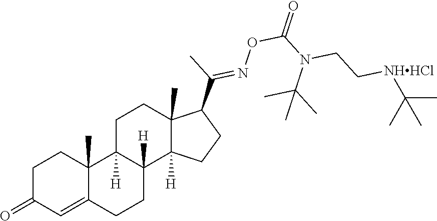

Prepared according to General Procedure B using commercially available N¹,N²-di-tert-butylethane-1,2-diamine, followed by treatment with HCl in dioxane (4 M). The product was in the form of a white solid (165 mg, 0.292 mmol, 59% yield). ¹H NMR (600 MHZ, DMSO) δ 9.21 (br s, 2H), 5.63 (s, 1H), 3.66 (t, J=8.4 Hz, 2H), 2.96-2.86 (m, 2H), 2.45-2.34 (m, 3H), 2.28-2.21 (m, 1H), 2.19-2.12 (m, 2H), 1.99-1.97 (m, 1H), 1.97 (s, 3H), 1.88-1.83 (m, 1H), 1.83-1.77 (m, 1H), 1.70-1.51 (m, 5H), 1.40 (s, 9H), 1.38-1.32 (m, 2H), 1.30 (s, 9H), 1.25-1.16 (m, 2H), 1.15 (s, 3H), 1.02-0.90 (m, 2H), 0.65 (s, 3H); ¹³C NMR (151 MHZ, DMSO) § 198.0, 170.9, 164.0, 153.2, 123.2, 56.4, 56.0, 56.0, 54.7, 53.1, 43.4, 41.0, 38.2, 37.8, 35.1, 35.1, 33.6, 31.9, 31.6, 28.7, 25.0, 23.7, 22.5, 20.6, 17.5, 16.9, 13.1. One 13C signal not observed. HRMS (APCI+) [M+H]⁺ calc. for C₃₂H₅₄N₃O₃, 528.41597, observed, 528.41599.

### AZ. Preparation of (E)-2-((8S,9S,10R,13S,14S,17S)-10,13-dimethyl-3-oxo-2,3,6,7,8,9,10,11,12,13,14,15,16,17-tetradecahydro-1H-cyclopenta[a]phenanthren-17-yl)-11-hydroxy-6-(2-hydroxyethyl)-4-oxa-3,6,9-triazaundec-2-en-5-one hydrochloride (54d)

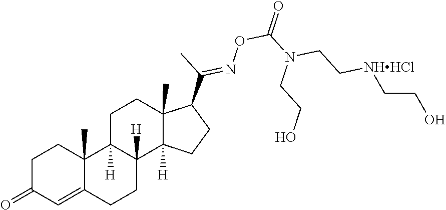

Prepared according to General Procedure B using commercially available 2,2′-(ethane-1,2-diylbis (azanediyl))bis(ethan-1-ol), followed by treatment with HCl in dioxane (4 M). The product was in the form of a white solid (163 mg, 0.302 mmol, 69% yield). ¹H NMR (600 MHZ, DMSO) δ 9.18-8.68 (m, 2H), 5.63 (s, 1H), 5.28 (t, J=5.0 Hz, 1H), 5.03 (s, 1H), 3.69-3.63 (m, 3H), 3.62-3.57 (m, 1H), 3.57-3.52 (m, 2H), 3.33-3.29 (m, 2H, masked by H₂O signal), 3.16-3.09 (m, 2H), 3.03-2.97 (m, 2H), 2.45-2.33 (m, 3H), 2.28-2.21 (m, 1H), 2.21-2.11 (m, 2H), 2.01-1.83 (m, 5H), 1.84-1.75 (m, 1H), 1.70-1.49 (m, 5H), 1.42-1.28 (m, 2H), 1.26-1.16 (m, 2H), 1.14 (s, 3H), 1.05-0.88 (m, 2H), 0.65 (s, 3H); ¹³C NMR (151 MHZ, DMSO) δ 198.0, 170.9, 164.7, 154.2, 123.2, 59.1, 56.3, 55.9, 54.7, 53.1, 50.1, 49.2, 45.3, 44.8, 44.2, 43.4, 38.2, 37.8, 35.1, 35.1, 33.6, 31.9, 31.6, 23.7, 22.5, 20.6, 16.9, 13.1. HRMS (APCI+) [M+H]⁺ calc. for C₂₈H₄₆N₃O₅, 504.3432, observed, 504.34374.

### Example 12. Bioanalytical Studies

### A. Materials and Methods

### Nephelometry

Nephelometry experiments were performed using untreated CORNING® COSTAR® 96-well black polystyrene plates with clear flat bottoms. Sample stock solutions and serial dilutions were prepared with DRISOLV® DMSO purchased from MilliporeSigma. All 100-fold dilutions and replicate experiments were prepared using GIBCO® Dulbecco's phosphate-buffered saline (DPBS) with a pH range of 7.0-7.3 as aqueous medium. Incubation of the 96-well plates was achieved with a Benchmark Incu-Shaker Mini Shaking Incubator. Nephelometry data was obtained using a NEPHELOSTAR® microplate reader and processed with the MARS data analysis software from BMG Lab Tech.

Tested compounds were dissolved in 100% DMSO to make stock solutions of specified concentrations, ranging from 10 mM minimum up to 75 mM maximum. The sample then underwent serial dilution in a 96-well plate. Well A1 of the plate contained 100% DMSO. Wells A2-A12 possessed the test compound in DMSO with concentration factors as follows (prepared via serial dilution with DMSO): X mM for A2, (0.8)X mM for A3, (0.6)X mM for A4, (0.4)X mM for A5, (0.2)X mM for A6, (0.1)X mM for A7, (0.05)X mM for A8, (0.025)X mM for A9, (0.0125)X mM for A10, (0.00625)X mM for All, and (0.003125)X mM for A12. Using a 12-channel multichannel pipette, 2.5 μL of sample from row A was transferred to each well in row B through row H of the plate. Next, 30 μL of DPBS was added to row B through row H, providing each well with 32.5 μL. The plate was then incubated for 30 sec with shaking. Finally, 217.5 μL of DPBS buffer was added to row B through row H, and the entire plate was incubated with shaking at 25° C. for 90 min. The final volume of DMSO in actual experiments with the DPBS buffer is 1% throughout the plate. After 90 min, the 96-well plate was analyzed with the NEPHELOSTAR® instrument and the data was processed with the MARS data analysis software.

### Aqueous Stability Assay

Aqueous stability experiments were performed on an Agilent 1100 HPLC equipped with an Agilent 1200 autosampler. HPLC grade solvents were exclusively used during analysis, with acetonitrile being purchased from Sigma Aldrich, and water and formic acid provided by Fisher Scientific. Sample solutions were prepared in 2.0 mL large opening RAM™ vials from Chemglass using DPBS as the aqueous medium. Filtration of undissolved material was achieved with Whatman Puradisc 4 mm PTFE (0.45 μm) syringe filters from Cytiva.

Tested compounds were dissolved in DPBS at a standard concentration of 1 mg/mL. All mixtures were then sonicated for 5 min or stirred until complete dissolution was achieved. Any precipitate observed in sample solutions was filtered with a PTFE syringe filter prior to analysis. Immediately after sample preparation, injection by the HPLC autosampler was performed, which was recorded as the initial time point (t=0 min). Samples were then kept in the autosampler at room temperature and injected at designated intervals without additional filtration or sample modification. Next, the area of the parent peak in each chromatogram was determined by automated integration in the Agilent ChemStation software. Pseudo first-order half-lives were then ascertained by calculating the percent of the parent compound remaining after each time point. Plots of time (x-axis) vs. the natural logarithm of percent parent remaining (y-axis) were subsequently constructed to determine the slope. Finally, the aqueous stability was evaluated using the following equation:

$${t}_{1 / 2} = ln ( 2 ) / - slope$$

The HPLC conditions were as follows: injection volume, 1 μL; column temperature: 40° C.; detector: 254 nm; stationary phase: Zorbax Eclipse XDB-C18 (4.6×150 mm, 5 μm); flow rate: 1 mL/min; mobile phase: 80-95% acetonitrile in H₂O (0.1% formic acid).

### Plasma Stability Assay

Procaine and procainamide were purchased from Sigma Aldrich. HPLC-grade acetonitrile, water, methanol, and formic acid were purchased from Fisher Scientific. Human plasma (Cat. No. HUMANPLLHP2N) was obtained from BIOIVT, and PBS (1×Dulbecco's, pH 7.4) from Thermo Fisher Scientific.

Test compounds were dissolved in DMSO to make a stock solution of 10 mM and then diluted to 500 μM in buffer or 70% methanol. Human plasma was thawed at ambient temperature and aliquoted (994.0 μL) to a 1.5 mL Eppendorf tube in duplicates (vials A and B) for each compound. The plasma was incubated at 37° C. for 10 min in an incubator shaker at 150 RPM; the reaction was initiated by addition of the test compound (6.0 μL), followed by vortex mixing. The total reaction volume was 1000 μL, the final organic solvent concentrations were 0.6% methanol (when 70% methanol was used for dilution) and 0.03% DMSO, and the final concentration of the test compound was 3 μM. The spiked plasma samples were incubated at 37° C. for 4 h. The reactions were terminated at time point 0, 15, 30, 60, 120, 180, and 240 min by taking a 100 μL aliquot from the test incubation mixture and immediately quenching it by adding it into ice-cold acetonitrile or methanol (150 μL) containing 2 μM internal standard (ISTD), followed by vortex mixing. The ISTD was d₅-7-ethoxy coumarin. The samples were then centrifuged at 15000 RPM for 25 min at 4° C., and the supernatant was transferred to an LC-MS vial for analysis by LC-MS/MS. Each time point was tested in duplicates followed by in-between blank washes to avoid carryover and to equilibrate the column.

Procaine (poor plasma stability) and procainamide (good plasma stability) were used as controls at the same concentration as that of the test compound. These controls were run in parallel to test the assay's competency. Matrix blank was prepared by adding acetonitrile or methanol containing ISTD to plasma samples without any of the test or control compounds. Also, an additional control sample was made to simply monitor compound degradation in PBS buffer.

Below is a summary of samples prepared for a given test compound in a typical plasma stability assay:
Test compound (TC): 994 μL human plasma+6.0 μL TC (Vial A) 994 μL human plasma+6.0 μL TC (Vial B)
Control: 596 μL human plasma+3.6 μL (procaine+procainamide)
Blank matrix: 500 μL PBS buffer+100 μL human plasma
Additional control: 142 μL PBS buffer+0.9 μL TC
Quenching mixture: 150 μL acetonitrile or methanol with ISTD (2 μM)
Final volume: 250 μL (100 μL from the incubation mixture+150 μL quenching mixture; final ISTD conc.: 1.2 μM)

LC-MS/MS analysis was performed using Agilent 1260 Infinity II HPLC, coupled with an Agilent G6460 triple quadrupole mass spectrometer (Agilent Technologies, USA). The data were acquired and processed using the Agilent 6460 Quantitative Analysis data processing software.

Reverse-phase HPLC separation for each compound was achieved on an Agilent InfinityLab Poroshell 120 C₁₈ column (2.1×50 mm, 2.7 μm) with a mobile phase composed of methanol/water with 0.1% formic acid or acetonitrile/water with 0.1% formic acid at a flow rate of 0.5 mL/min. Each method was developed in the presence of the ISTD. The column temperature was maintained at 40° C. The detection was operated using the Agilent Jet-Stream electrospray positive ionization under the multiple reaction monitoring mode. The mass spec conditions were as follows: dwell time 100 ms; gas flow 10 L/min; nebulizer pressure 45 psi; delta EMV 200 V.

Plots of time (x-axis) vs. the natural logarithm of percent parent remaining (y-axis) were subsequently constructed to determine the slope. Finally, the plasma stability was evaluated using the following equation:

$${t}_{1 / 2} = ln ( 2 ) / - slope$$

### B. Results

Results of aqueous stability (determined in DPBS, pH 7.0-7.3) and human plasma stability were summarized in Table 1.

TABLE 1
Stability of prodrugs of neurosteroid analogs in PBS and human plasma

| Com- | | | PBS | Plasma | Aqueous |
|---|---|---|---|---|---|
| pound | | | t_{1/2} | t_{1/2} | solubility |
| name | # | Structure | (h) | (h) | (μM) |
| RGF- PROG-35 | 32a | 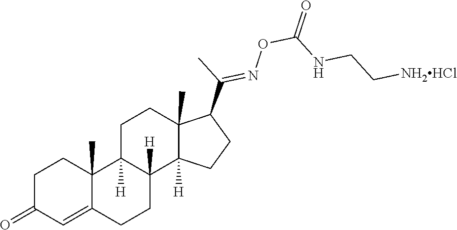 | >30 | >2 | >750 |
| RGF- PROG-44 | 32b | 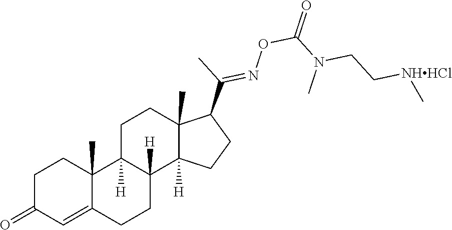 | 46.9 ± 0.8 | 1.5 | >750 |
| AVW- PROG-80 | 32c | 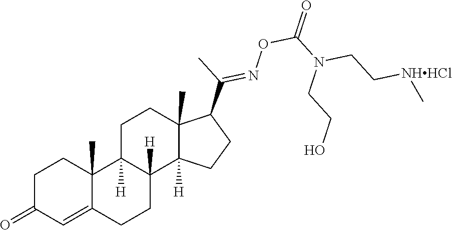 | 24.8 ± 1.1 | 1.7 | >500 |
| AVW- PROG-87 | 32d | 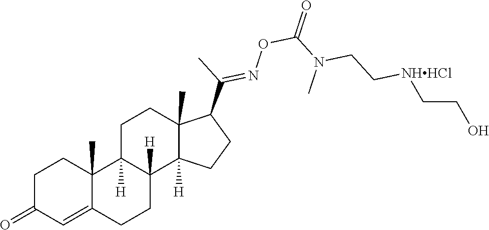 | 30.4 ± 5.0 | 2.8 | >500 |
| RGF- PROG-46 | 32e | 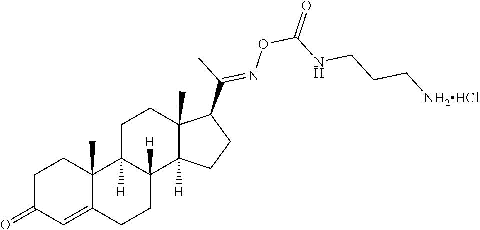 | 2880 (120 d) | N/A | N/A |
| RGF- PROG-37 | 32f | 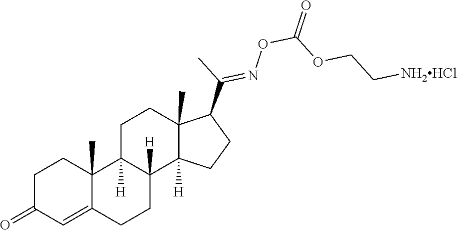 | 0.11 ± 0.01 | N/A | N/A |
| RGF- PROG-83 | 32g | 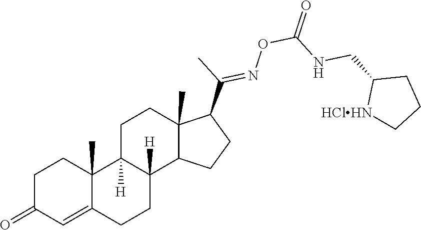 | 16.5 ± 0.5 d | 9.6 | >750 |
| AVW- PROG-91 | 32h | 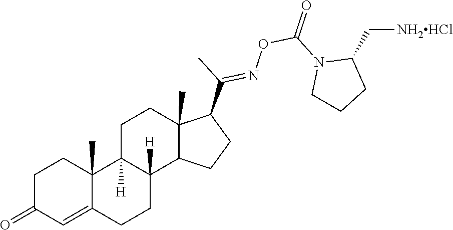 | >30 d | >4 | N/A |
| RGF- PROG-84 | 32i | 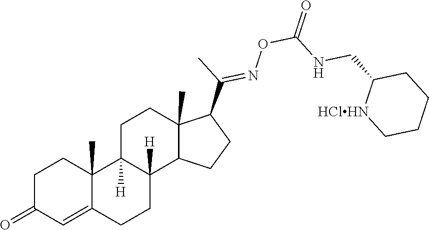 | 28.5 ± 1.2 d | N/A | >750 |
| RGF- PROG-104 | 32j | 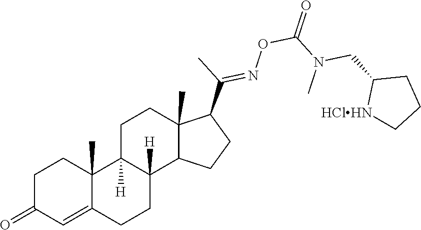 | 1.9 ± 0.1 | <0.1 | N/A |
| AVW- PROG-94 | 32k | 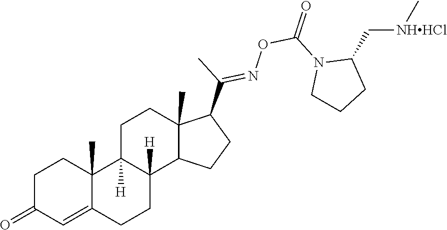 | >30 d | >4 | N/A |
| RGF- PROG-111 | 32l | 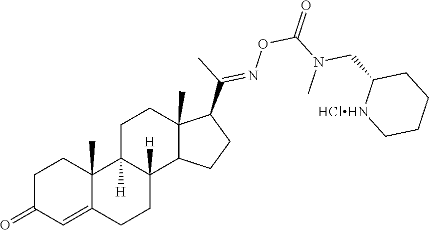 | 19.8 ± 1.5 d | 6.8 | N/A |
| RGF- PROG-215 | 32m | 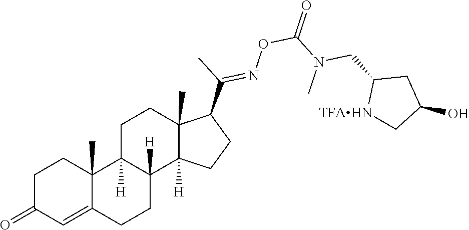 | 1.3 | N/A | N/A |
| RGF- PROG-78 | 32n | 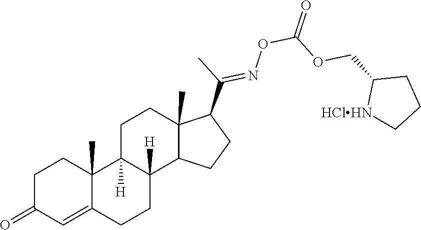 | 0.09 ± 0.01 | N/A | N/A |
| AVW- PROG-110 | 32o | 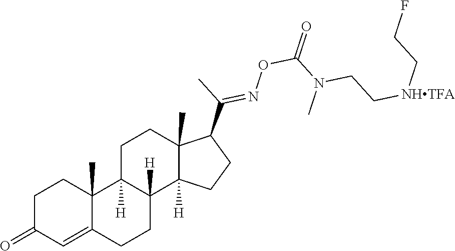 | 36.1 ± 2.4 | 2.2 | N/A |
| RGF- PROG-49 | 33 | 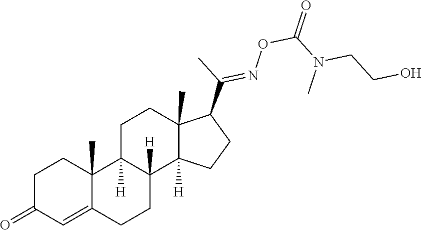 | 38.5 | N/A | 123.9 ± 3.28 |
| RGF- PROG-45 | 34 | 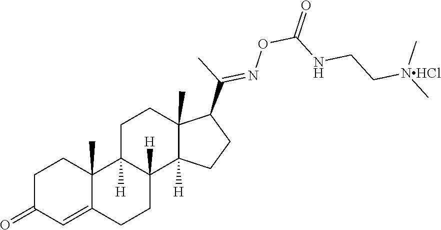 | 32.8 ± 3.3 d | >3.5 | >750 |
| RGF- PROG-48 | 35 | 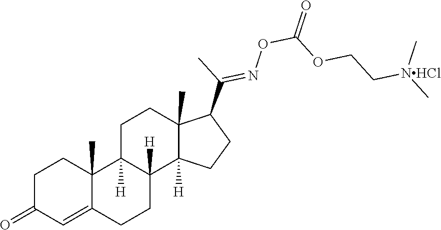 | 3.5 ± 0.6 | 7.2 | N/A |
| RGF- PROG-167 | 36 | 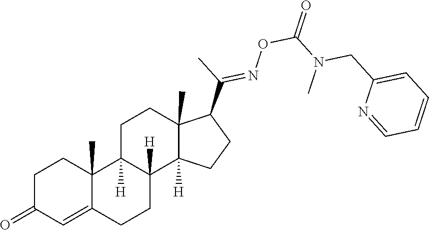 | N/A | 222 | N/A |
| RGF- PROG-168 | 37 | 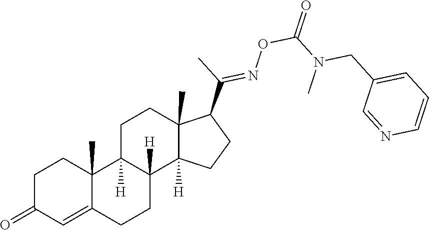 | N/A | N/A | N/A |
| RGF- PROG-054 | 38 | 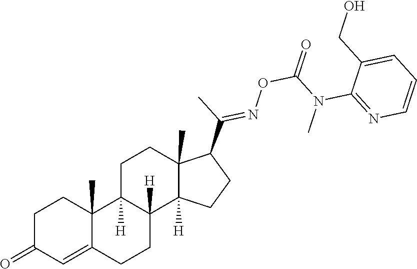 | N/A | N/A | N/A |
| RGF- PROG-255 | 39 | 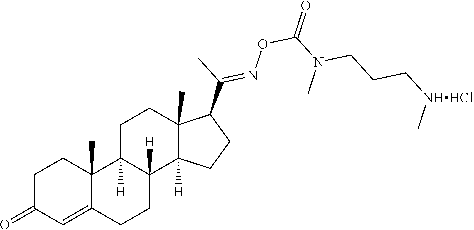 | >30 d | >4 | N/A |
| AVW- PROG-137 | 40 | 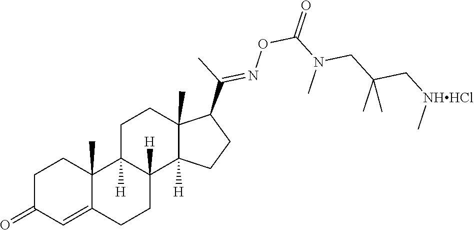 | 22.8 ± 0.8 d | >4 | N/A |
| AVW- PROG-142 | 41 | 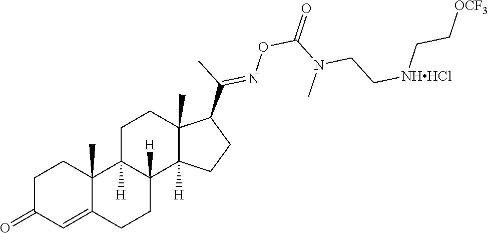 | 28.5 ± 1.8 | 1.1 | N/A |
| Com- | | | PBS | Plasma | Aqueous |
| pound | | | t_{1/2} | % rmn | solubility |
| name | # | Structure | (h) | at 2 h | (μM) |
| AVW- PROG-185 | 53a | 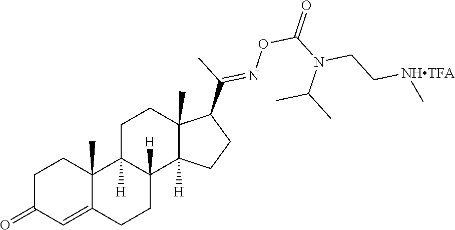 | 19.72 ± 0.38 | 41% | >750 |
| AVW- PROG-183 | 53b | 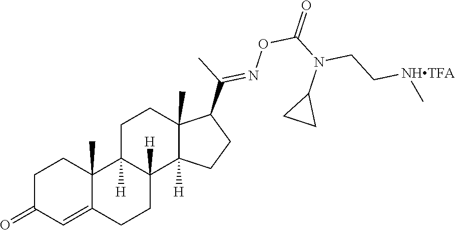 | 28.16 ± 0.04 | 45% | >750 |
| DRL- PROG-21 | 53c | 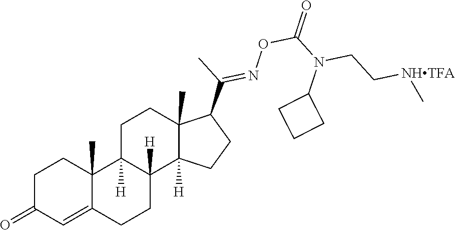 | 11.79 ± 0.51 | N/A | >750 |
| AVW- PROG-184 | 53d | 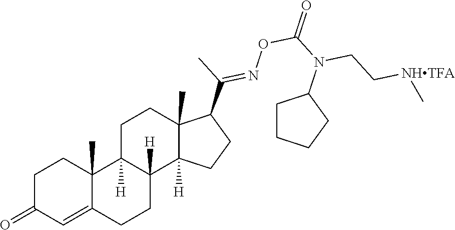 | 10.70 ± 0.15 | 65% | >750 |
| AVW- PROG-186 | 53e | 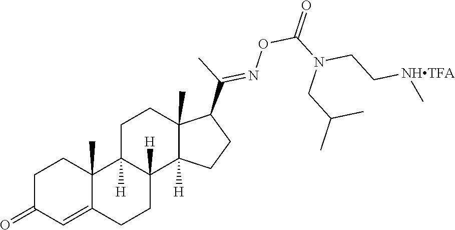 | >24 | 83% | >750 |
| LEH- PROG-12 | 53f | 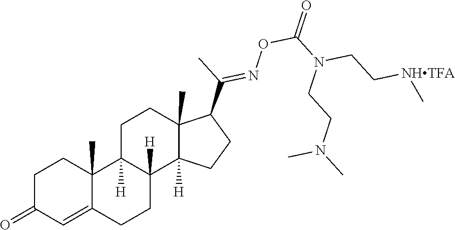 | 2.17 ± 0.02 | N/A | >500 |
| DRL- PROG-26 | 53g | 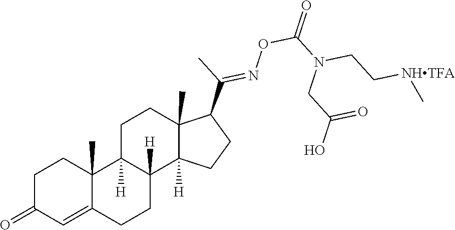 | >24 h | N/A | N/A |
| DRL- PROG-27 | 53h | 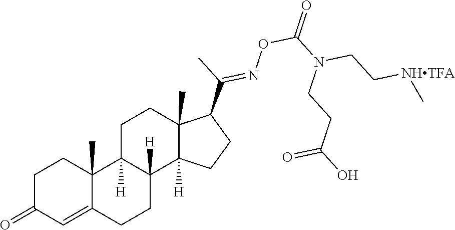 | >24 h | N/A | N/A |
| AVW- PROG-173 | 53i | 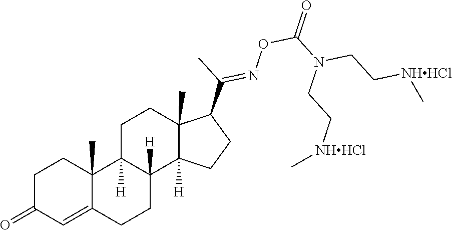 | 1.51 ± 0.05 | N/A | >750 |
| AVW- PROG-175 | 53j | 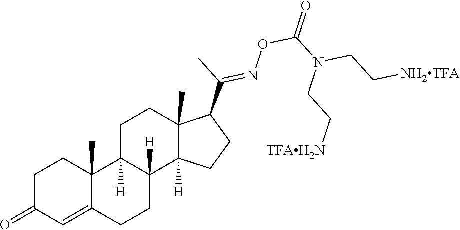 | 10.13 ± 0.41 | N/A | >750 |
| AVW- PROG-199 | 53k | 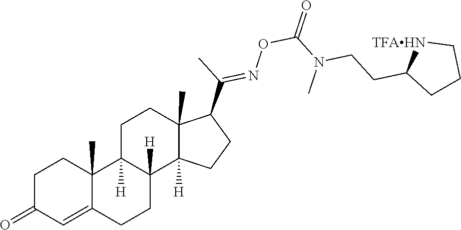 | N/A | N/A | N/A |
| RGF- PROG-263 | 54a | 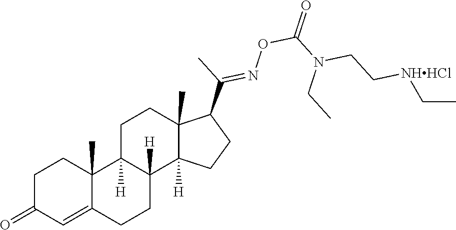 | >24 h | 71% | >750 |
| AVW- PROG-162 | 54b | 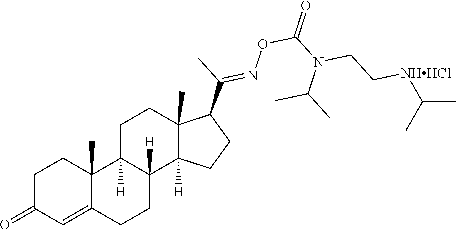 | >24 h | 84% | >500 |
| AVW- PROG-170 | 54c | 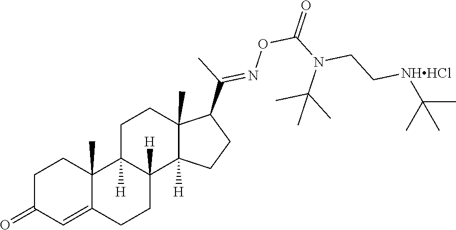 | >24 h | 93% | N/A |
| RGF- PROG-264 | 54d | 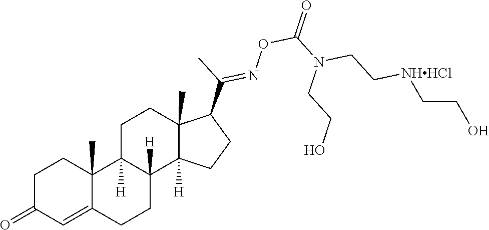 | 28.79 ± 3.16 | 32% | >750 |

N/A: not available

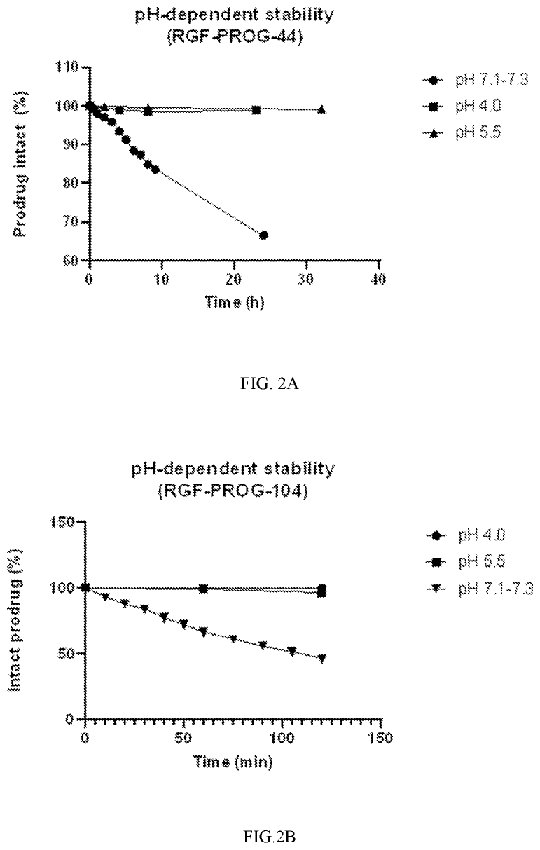

The stability of compounds 32b and 32j in acidic aqueous mediums (pH 4.0 and 5.5) was also determined (FIGS. 2A and 2B) and the t_{1/2} values are summarized in Table 2.

TABLE 2
Stability of compounds 32b and 32j in different aqueous mediums

| | | PBS | Acetate buffer | Acetate buffer |
|---|---|---|---|---|
| Compound | | (pH 7.0-7.3) | (pH 5.5) | (pH 4.0) |
| name | # | t_{1/2} (h) | t_{1/2} (d) | t_{1/2} (d) |
| RGF-PROG-44 | 32b | 46.9 ± 0.8 | 80 | 96 |
| RGF-PROG-104 | 32j | 1.9 ± 0.1 | 2.4 | 48 |

N/A: not available

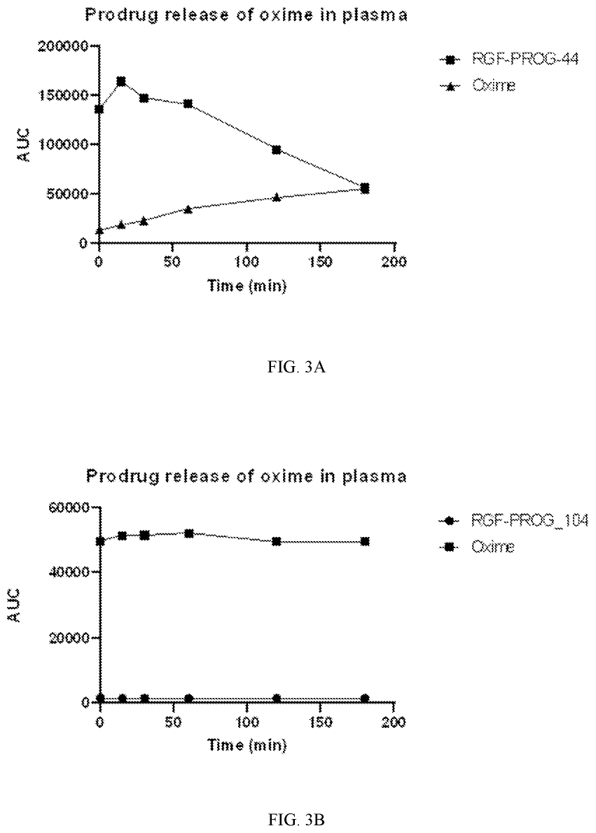

Compounds 32b and 32j are stable in the acidic aqueous mediums (Table 2) but are unstable in human plasma (Table 1). As illustrated in FIG. 1, formation of the corresponding oxime (i.e., progesterone C20-oxime) was observed in human plasma (FIGS. 3A and 3B).

### Example 13. In Vivo Studies Using an Animal Model of TBI

### A. Materials and Methods

With evidence of prodrug conversion to the parent compound, compounds 32b and 32j were evaluated side by side with EIDD-1723 (see below) in a rat disease model of acute TBI (Wali, B., Sayeed, I., Guthrie, D. B., Natchus, M. G., Turan, N., Liotta, D. C., Stein, D. G., Neuropharmacology, 2016, 109, 148-158; Sayeed, I., Wali, B., Guthrie, D. B., Saindane, M. T., Natchus, M. G., Liotta, D. C., Stein, D. G., Neuropharmacology, 2019, 145, 292-298). A prodrug dose of 10 mg/kg was chosen and administered IM at 1 and 6 hours post-injury. Prodrug dosage is equimolar±5% with respective to the parent compound.

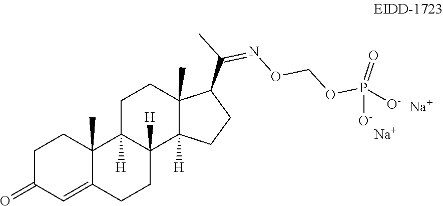

Subjects—Male SD rats weighing 250-350 g at the time of injury were used for the efficacy study, which was conducted in our facility at Emory University. The rats were maintained on a reverse 12:12 light-dark cycle at 22±1° C. with appropriate humidity levels. After one week of quarantine the rats were handled at least 3 times prior to surgery. This study was conducted in a facility approved by the American Association for the Accreditation of Laboratory Animal Care (AAALAC) in accordance with all National Institutes of Health guidelines. All experimental animal procedures were approved by the Emory University Institutional Animal Care and Use Committee.

Contusion Injury—Rats were anesthetized using isoflurane gas (5% induction, 1.5% maintenance, 700 mmHg N₂O, 500 mmHg O₂) and then mounted in a Kopf stereotaxic device (David Kopf Instruments, Tujunga, CA, USA). The scalp incision area was shaved and sterilized with Betadine® antiseptic and 70% isopropanol. Physiological parameters were monitored with pulse oximetry (SurgiVet model V3304): heart rate was maintained above ˜300 beats per minute and SpO2 kept above 90%. Core body temperature (˜37° C.) was maintained with a homeothermic heating blanket system (Harvard Apparatus, Holliston, MA, USA). Under aseptic conditions, a midline incision was made into the skin and fascia covering the skull. Cotton swabs were used to staunch and clean any fascial bleeding. Bregma was located and a trephine drill was used to perform a 5-mm diameter mid-sagittal bilateral craniotomy 3 mm anterior to bregma. Controlled cortical impact (CCI) injury to the medial frontal cortex (MFC) was induced with a magnetic cortical pinpoint contusion impactor (4-mm diameter; PC1300; Hatteras Instruments, Cary, NC, USA) to a depth of 2.5 mm at a pressure of 1.7 psi, impact time of 100 ms, and velocity of 2.26 m/s. Sutures were used to close the incision after bleeding stopped. Animals were then placed into heated recovery boxes and allowed to recover from the anesthesia before being returned to their home cages. The sham group received no impact, and the incisions were sutured closed after comparable time under anesthesia.

Treatment—Animals were randomly assigned to one of four treatment groups. For the edema assay: sham (n=4), vehicle (normal saline; n=4), test compounds (10 mg/kg; n=4). Treatment was administered IM at 1 and 6 h post-TBI. Animals were euthanized at 24 h post-injury and their brains removed and sampled for edema assay. Percent change in brain tissue water content was measured.

Edema Assay—Edema was measured at 24 h after surgery. Briefly, the rats were decapitated under deep anesthesia and the brain extracted and dissected into anterior and posterior sections. The anterior section contained the entire lesion area. Each section was placed in a prelabeled and pre-weighed tube that was immediately capped. Each tube was reweighed, and then opened and placed in a 60° C. oven with 15 mmHg vacuum pressure for 48 h. Samples were reweighed after drying. All weighing was done on the same balance. The percent water content was calculated by [(wet wt-dry wt)/wet wt]×100. The percent difference in water content between the anterior peri-contusional and the posterior distal section was calculated for each sample by: [(anterior H₂O %-posterior H₂O %)/(posterior H₂O %)]×100.

### B. Results

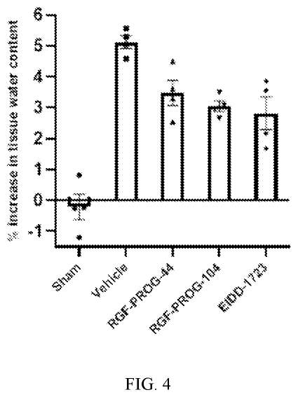

Treatment with either compound 32b or 32j reduced brain edema (31% and 40%, respectively) following acute TBI in rats (FIG. 4). Comparison with EIDD-1723 revealed that 32j was equally efficacious in reducing brain edema when administered at the 10 mg/kg dose.

### Example 14. Pharmacokinetic Studies

### A. Materials and Methods

Rat pharmacokinetic studies was outsourced to Sai Life Sciences.

Test System—Healthy male Sprague Dawley rats (8-12 weeks old) weighing between 200 to 250 g were procured from Global, India. Three mice were housed in each cage. Temperature and humidity were maintained at 22±3° C. and 30-70%, respectively and illumination was controlled to give a sequence of 12 h light and 12 h dark cycle. Temperature and humidity were recorded by auto-controlled data logger system. All the animals were provided with laboratory rodent diet (Envigo Research private Ltd, Hyderabad). Reverse osmosis water treated with ultraviolet light was provided ad libitum.

Design—Rats (n=3 per treatment group, serial sampling) were administered a solution of either normal saline vehicle or test compound in normal saline at 2 mg/kg (IV) or 10 mg/kg (IM) with a dosing volume of 5 mL/kg (IV) or 1 mL/kg (IM).

Sample Collection—Blood samples (approximately 60 μL) were collected under light isoflurane anesthesia (Surgivet®) from retro orbital plexus from a set of three rats at 0.08, 0.25, 0.5, 1, 2, 4, 6, 8 and 10 h (IV) or 0.25, 0.5, 1, 2, 4, 6, 8 and 10 h (IM). Immediately after blood collection, plasma was harvested by centrifugation 4000 rpm, 10 min at 40° C. and samples were stored at −70±10° C. until bioanalysis. Prior to the study, the test compound was assessed for ex vivo conversion in rat plasma under these conditions and were determined to be stable under the storage conditions described.

Data Analysis—Concentrations of the test compound and its parent compound, progesterone C20-oxime, in rat plasma were determined by fit-for-purpose LC-MS/MS method by Sai Life Sciences. Analysis of these data were conducted at Emory University. Cₘₐₓ represents the mean observed maximum concentration of analyte. Tₘₐₓ represents the average time at observed Cₘₐₓ. Areas under the curve (AUC₀₋ₗₐₛₜ) were calculated via the linear trapezoidal rule using GraphPad Prism v9.

Ethical Declaration—This study was approved by the Institutional Animal Ethics Committee (IAEC) and performed in accordance with the guidelines provided by the Committee for the Purpose of Control and Supervision of Experiments on Animals (CPCSEA) as published in The Gazette of India, Dec. 15, 1998.

### B. Results

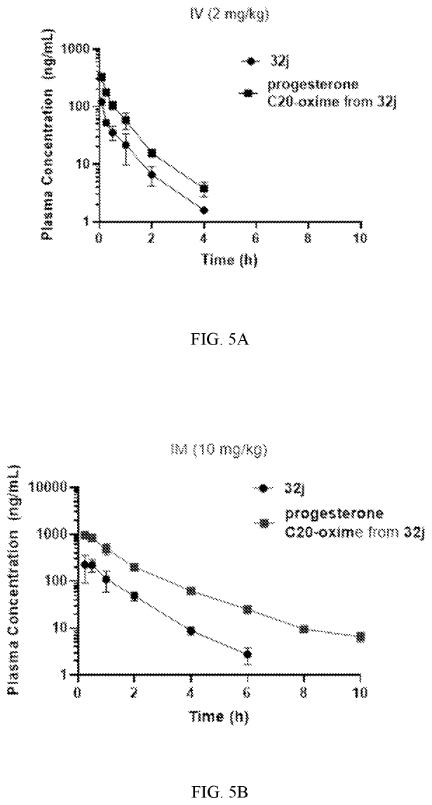

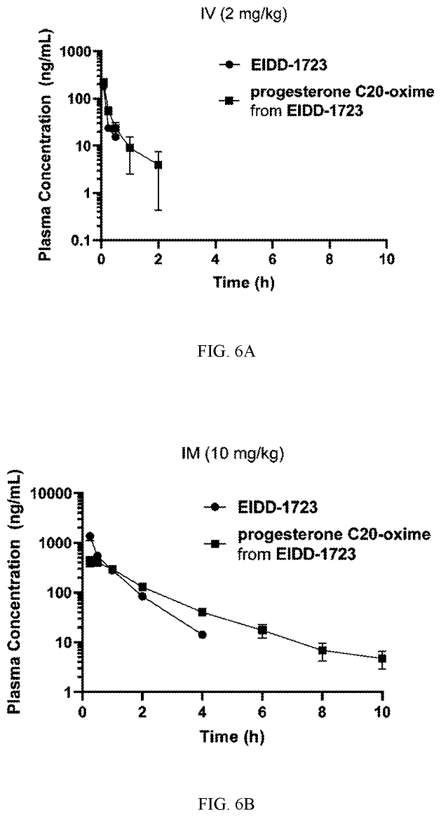

The rat pharmacokinetics (PK) of prodrug 32j was determined in a side-by-side study with EIDD-1723 (FIGS. 5A, 5B, 6A, and 6B). Results are summarized in Table 3. Male rats were dosed with either 32j or EIDD-1723 via IV (2 mg/kg) or IM (10 mg/kg) route. IM administration of EIDD-1723 resulted in a 3-fold increase in the dose-corrected exposure to parent progesterone C20-oxime compared to the IV route. The pH-responsive prodrug 32j exhibited rapid and extensive formation of parent progesterone C20-oxime when administered via IV and IM routes (FIGS. 5A and 5B). An accurate half-life could not be determined for the prodrug 32j due to its rapid conversion rate to parent progesterone C20-oxime. As shown in Table 3, 32j afforded 80% (IM) and 179% (IV) greater exposure of parent progesterone C20-oxime compared to EIDD-1723 as determined by the AUC.

TABLE 3
Pharmacokinetic summary of prodrugs 32j and EIDD-1723

| | | | | Prodrug | Parent |
|---|---|---|---|---|---|
| | Route | Prodrug Cₘₐₓ | Parent Cₘₐₓ | AUCₗₐₛₜ | AUCₗₐₛₜ |
| | (dose) | (ng/mL) | (ng/mL) | (h*ng/mL) | (h*ng/mL) |
| 32j | IM | 224 | 954 | 288 ± 40 | 1304 ± 89 |
| | (10 mg/kg) | | | | |
| | IV | 122 | 328 | 62.9 ± 8 | 176 ± 13 |
| | (2 mg/kg) | | | | |
| EIDD-1723 | IM | 1362 | 425 | 724 ± 39 | 753 ± 29 |
| | (10 mg/kg) | | | | |
| | IV | 182 | 219 | 22 ± 1 | 48 ± 6 |
| | (2 mg/kg) | | | | |

What is claimed is:

1. A compound of Formula II or a pharmaceutically acceptable salt, hydrate, or hydrated salt thereof, 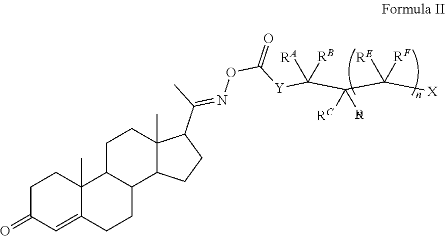
wherein n is 0 or 1; and
wherein:
(1) X is OH or NR¹R²,
Y is O or NR³,
R^{A}, R^{B}, R^{C}, R^{D}, R^{E}, and R^{F} are independently selected from the group consisting of hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R¹, R², and R³ are independently selected from the group consisting of hydrogen, optionally substituted alkyl, optionally substituted heteroalkyl, optionally substituted carbocyclyl, optionally substituted heterocyclyl, optionally substituted aryl, and optionally substituted heteroaryl, with the proviso that at least one of R¹ and R² is hydrogen;
(2) X is NR¹R²,
Y is O or NR³,
R¹ joins R^{C} or R^{E} to form a 4-7 membered, optionally substituted heterocycle,
R² is hydrogen,
R^{A}, R^{B}, R^{C}, R^{D}, R^{E}, and R^{F}, on each occurrence when not joined by R¹ to form the heterocycle, are independently selected from the group consisting of hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R³ is independently selected from the group consisting of hydrogen, optionally substituted alkyl, optionally substituted heteroalkyl, optionally substituted carbocyclyl, optionally substituted heterocyclyl, optionally substituted aryl, and optionally substituted heteroaryl; or
(3) X is OH or NR¹R²,
Y is NR³,
R³ joins R^{A} to form a 4-7 membered, optionally substituted heterocycle,
R^{B}, R^{C}, R^{D}, R^{E}, and R^{F} are independently selected from the group consisting of hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R¹ and R² are independently selected from the group consisting of hydrogen, optionally substituted alkyl, optionally substituted heteroalkyl, optionally substituted carbocyclyl, optionally substituted heterocyclyl, optionally substituted aryl, and optionally substituted heteroaryl, with the proviso that at least one of R¹ and R² is hydrogen.

2. The compound of claim 1, wherein the compound has a structure of Formula II-1 or is a pharmaceutically acceptable salt, hydrate, or hydrated salt thereof, 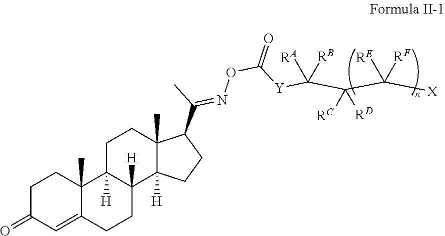
wherein X, Y, R^{A}, R^{B}, R^{C}, R^{D}, R^{E}, R^{E}, and n are the same as described in claim 1.

3. The compound of claim 1, wherein:
X is OH or NR¹R²,
Y is O or NR³,
R^{A}, R^{B}, R^{C}, R^{D}, R^{E}, and R^{E} are independently selected from the group consisting of hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R¹, R², and R³ are independently selected from the group consisting of hydrogen, optionally substituted alkyl, optionally substituted heteroalkyl, optionally substituted carbocyclyl, optionally substituted heterocyclyl, optionally substituted aryl, and optionally substituted heteroaryl, with the proviso that at least one of R¹ and R² is hydrogen.

4. The compound of claim 3, wherein n is 0.

5. The compound of claim 3, wherein X is NR¹R², wherein R¹ is optionally substituted C₁-C₄ alkyl and R² is hydrogen.

6. (canceled)

7. (canceled)

8. The compound of claim 3, wherein Y is NR³, wherein R³ is optionally substituted C₁-C₄ alkyl.

9. (canceled)

10. (canceled)

11. The compound of claim 3, wherein Y is NR³, wherein R³ is optionally substituted carbocyclyl or optionally substituted heterocyclyl.

12-16. (canceled)

17. The compound of claim 3, selected from: 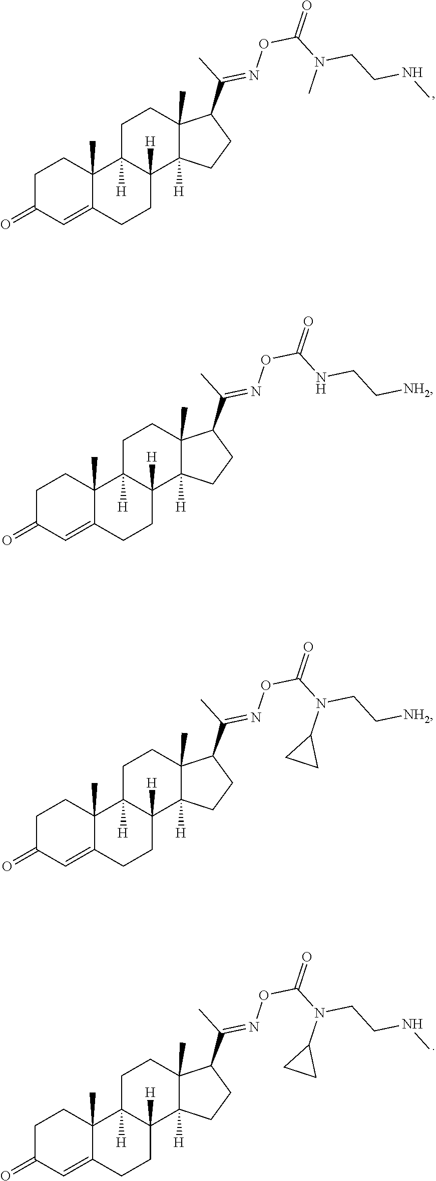
and pharmaceutically acceptable salts thereof.

18. (canceled)

19. The compound of claim 1, wherein:
X is NR¹R²,
Y is O or NR³,
R¹ joins R^{C} or R^{E} to form a 4-7 membered, optionally substituted heterocycle,
R² is hydrogen,
R^{A}, R^{B}, R^{C}, R^{D}, R^{E}, and R^{F}, on each occurrence when not joined by R¹ to form the heterocycle, are independently selected from the group consisting of hydrogen, halogen, cyano, nitro, carboxyl, carbonate, optionally substituted alkyl, optionally substituted alkenyl, optionally substituted alkynyl, optionally substituted carbocyclyl, optionally substituted heterocyclyl, optionally substituted aryl, optionally substituted heteroaryl, acyl, sulfinyl, sulfonyl, sulfonate, sulfonamide, amide, optionally Si-substituted silyl, ester, thioester, carbonate ester, and carbamate, and
R³ is independently selected from the group consisting of hydrogen, optionally substituted alkyl, optionally substituted heteroalkyl, optionally substituted carbocyclyl, optionally substituted heterocyclyl, optionally substituted aryl, and optionally substituted heteroaryl.

20. The compound of claim 19, wherein n is 0 or 1.

21-23. (canceled)

24. The compound of claim 19, wherein the 4-7 membered, optionally substituted heterocycle is optionally substituted pyrrolidine or optionally substituted piperidine.

25. The compound of claim 19, wherein Y is NR³, wherein R³ is optionally substituted C₁-C₄ alkyl.

26. (canceled)

27. (canceled)

28. The compound of claim 19, wherein Y is NR³, wherein R³ is optionally substituted carbocyclyl or optionally substituted heterocyclyl.

29-33. (canceled)

34. The compound of claim 19, selected from: 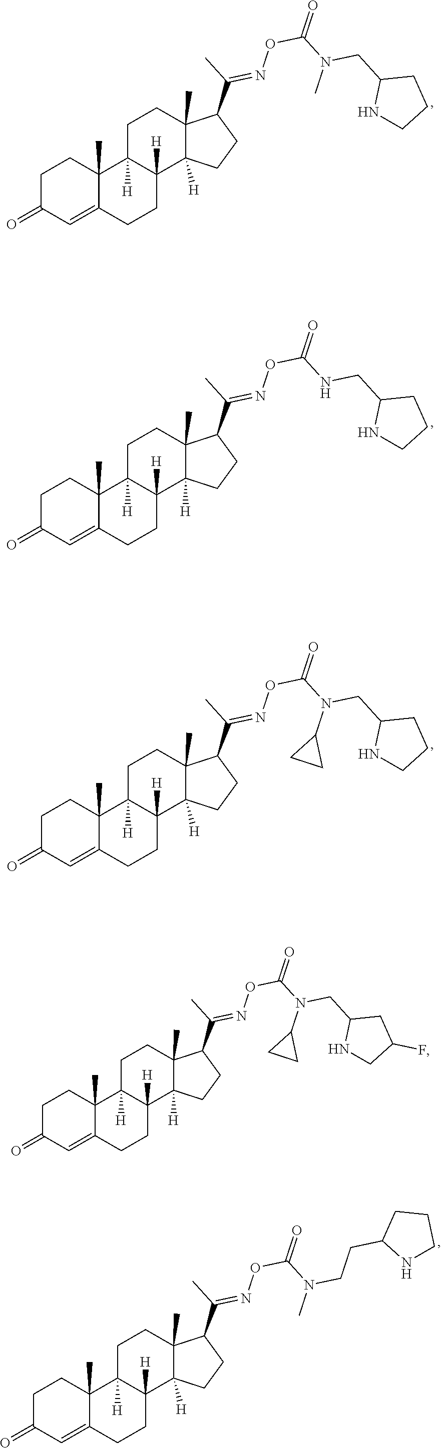 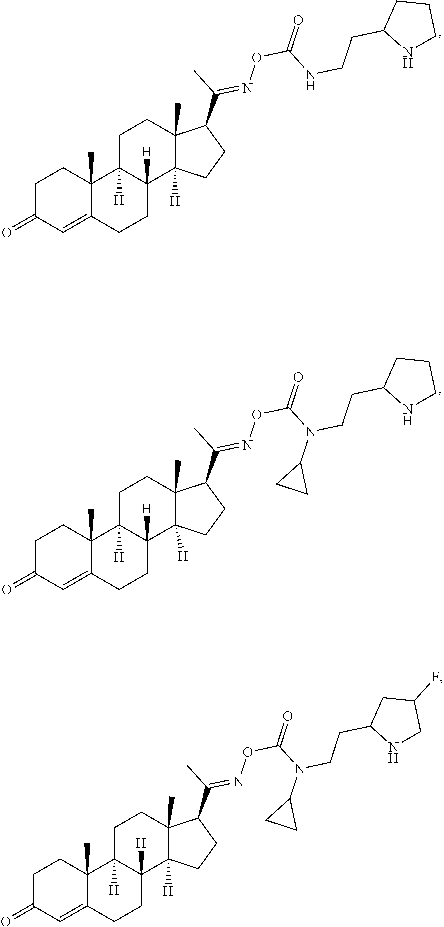
and pharmaceutically acceptable salts thereof.

35-59. (canceled)

60. The compound of claim 1, wherein the compound is in the form of an HCl salt.

61. A pharmaceutical formulation, comprising the compound of claim 1 and a pharmaceutically acceptable carrier.

62. The pharmaceutical formulation of claim 61, wherein the pharmaceutical formulation is in the form of tablet, capsule, pill, gel, cream, granule, solution, suspension, emulsion, or nanoparticulate formulation.

63-66. (canceled)

67. A method of treating a CNS condition or disorder in a subject in need thereof, comprising administering an effective amount of the compound of claim 1 to the subject.

68. The method of claim 67, wherein the compound is administered orally, intravenously, intranasally, or intramuscularly.

69. The method of claim 67, wherein the CNS condition or disorder is selected from stroke, subarachnoid hemorrhage, traumatic brain injury, concussion, dementia, Alzheimer's diseases, epilepsy, seizure disorder, depression, and postpartum depression.
